(19) United States
(12) Patent Application Publication
(10) Pub. No.: US 2005/0101595 A1
(43) Pub. Date: May 12, 2005

(54) N-containing cycloalkyl-substituted amino-thiazole derivatives and pharmaceutical compositions for inhibiting cell proliferation and methods for their use

(75) Inventors: Shao Song Chu, Encinitas, CA (US); Larry Andrew Alegria, Gallatin Gateway, MT (US); Ted Michael Bleckman, La Jolla, CA (US); Wesley K. M. Chong, Encinitas, CA (US); Rohit Kumar Duvadie, San Diego, CA (US); Lin Li, San Diego, CA (US); William Henry Romines III, San Diego, CA (US); Yi Yang, San Diego, CA (US)

Correspondence Address: AGOURON PHARMACEUTICALS, INC., 10350 NORTH TORREY PINES ROAD, LA JOLLA, CA, 92037, US

(73) Assignee: PFIZER INC.

(21) Appl. No.: 10/783,887

(22) Filed: Feb. 20, 2004

Related U.S. Application Data
(60) Provisional application No. 60/448,843, filed on Feb. 21, 2003.

(51) Int. Cl. A61K 31/55; A61K 31/454; A61K 31/427; C07D 417/02

(52) U.S. Cl. 514/217.09; 514/326; 514/370; 540/603; 546/209; 548/191

(57) ABSTRACT

Aminothiazole compounds with N-containing cycloalkyl at the 2-amino position which are represented by the Formula (I), or a pharmaceutically acceptable prodrug of said compound, pharmaceutically active metabolite or pharmaceutically acceptable salt of said compound, or metabolite thereof, modulate and/or inhibit the cell proliferation and activity of protein kinases. 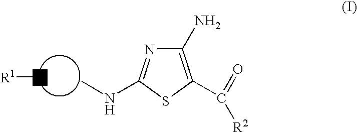 The invention is also directed to the therapeutic or prophylactic use of pharmaceutical compositions containing such compounds, and to methods of treating malignancies and other disorders by administering effective amounts of such compounds.

This application claims the benefit of U.S. Provisional Application Ser. No. 60/448,843, filed Feb. 21, 2003, and U.S. patent application Ser. No. 10/768,437 filed Jan. 30, 2004, the contents of which are hereby incorporated by reference in their entireties.

### FIELD OF THE INVENTION

This invention is directed to compounds with N-containing cycloalkyl-substituted aminothiazole nuclei that demonstrate an anti-proliferative activity such as antitumor activity, to processes for preparing these compounds and to pharmaceutical compositions containing such compounds. The invention is also directed to the therapeutic or prophylactic use of such compounds and compositions, and to methods of treating cancer, viral, microbial, and/or parasitic colonization/infection as well as other disease states associated with unwanted cellular proliferation, by administering effective amounts of such compounds.

### BACKGROUND OF THE INVENTION

Cell proliferation occurs in response to various stimuli and may stem from de-regulation of the cell division cycle (or cell cycle), the process by which cells multiply and divide. Hyperproliferative disease states, including cancer, are characterized by cells rampantly winding through the cell cycle with uncontrolled vigor due to, for example, damage to the genes that directly or indirectly regulate progression through the cycle. Thus, agents that modulate the cell cycle, and thus hyperproliferation, could be used to treat various disease states associated with uncontrolled or unwanted cell proliferation. In addition to cancer chemotherapeutic agents, cell cycle inhibitors are also proposed as antiparasitics (See, Gray et al., Curr. Med. Chem. 6, 859-875 (1999)) and recently demonstrated as potential antivirals (See, Schang et al., J. Virol. 74, 2107-2120 (2000)). Moreover, the applicability of antiproliferative agents may be expanded to treating cardiovascular maladies such as artherosclerosis or restenosis (See Braun-Dullaeus et al., Circulation, 98, 82-89 (1998)), and states of inflammation, such as arthritis (See, Taniguchi et al., Nature Med., 5, 760-767(1999)) or psoriasis. Recently, chemotherapy induced alopecia was alleviated in rats. (See Davis, et al., Science, 291, 134-137 (2001).

Mechanisms of cell proliferation are under active investigation at cellular and molecular levels. At the cellular level, de-regulation of signaling pathways, loss of cell cycle controls, unbridled angiogenesis or stimulation of inflammatory pathways are under scrutiny, while at the molecular level, these processes are modulated by various proteins, among which protein kinases are prominent suspects. Overall abatement of proliferation may also result from programmed cell death, or apoptosis, which is also regulated via multiple pathways, some involving proteolytic enzyme proteins.

Among the candidate regulatory proteins, protein kinases are a family of enzymes that catalyze phosphorylation of the hydroxyl group of specific tyrosine, serine or threonine residues in proteins. Typically, such phosphorylation dramatically perturbs the function of the protein, and thus protein kinases are pivotal in the regulation of a wide variety of cellular processes.

Cyclin-dependent kinases (CDKs) are serine-threonine protein kinases that play critical roles in regulating the transitions between different phases of the cell-cycle, such as the progression from a quiescent stage in G₁ (the gap between mitosis and the onset of DNA replication for a new round of cell division) to S (the period of active DNA synthesis), or the progression from G₂ to M phase, in which active mitosis and cell-division occurs. (See, e.g., the articles compiled in Science, 274, 1643-1677 (1996); and Ann. Rev. Cell Dev. Biol., 13, 261-291 (1997)). CDK complexes are formed through association of a regulatory cyclin subunit (e.g., cyclin A, B1, B2, D1, D2, D3, and E) and a catalytic kinase subunit (e.g., CDK1, CDK2, CDK4, CDK5, and CDK6). As the name implies, the CDKs display an absolute dependence on the cyclin subunit in order to phosphorylate their target substrates, and different kinase/cyclin pairs function to regulate progression through specific phases of the cell-cycle.

Aberrations in this control system, particularly those that affect the function of CDK4 and CDK2, have been implicated in the advancement of cells to the highly proliferative state characteristic of malignancies, particularly familial melanomas, esophageal carcinomas, and pancreatic cancers. (See, e.g., Hall et al., Adv. Cancer Res., 68, 67-108 (1996); Kamb, Trends in Genetics, 11, 136-140 (1995); Kamb et al., Science, 264, 436-440 (1994)).

Because CDK4 may serve as a general activator of cell division in most cells and complexes of CDK4/cyclin D and CDK2/cyclin E govern the early G1 phase of the cell cycle, CDK4 or CDK2 inhibitors may be used as anti-proliferative agents. Also, the pivotal roles of cyclin E/CDK2 and cyclin B/CDK1 in the G1/S phase and G2/M transitions, respectively, offer additional targets for therapeutic intervention in suppressing deregulated cell cycle progression.

A large number of small molecule ATP-site antagonists have been identified as CDK inhibitors. (See, Webster, Exp. Opin. Invest. Drugs, 7, 865-887 (1998), Stover, Et al., Curr. Opin. Drug Disc. Dev., 2, 274-285(1999), Gray et al., Curr. Med. Chem., 6, 859-875 (1999), Sielecki, et al., J. Med. Chem., 43, 1-18 (2000), Crews, et al., Curr. Opin. Chem. Biol., 4, 47-53 (2000), Buolamwini, Curr. Pharm. Des., 6, 379-392 (2000), Rosania, et al., Exp. Opin. Ther. Pat., 10, 215-230 (2000), fisher, et al., Curr. Med. Chem., 7, 1213-1245 (2000), and Fry, et al., Exp. Opin. Oncol. Endocrine Metab. Invest. Drugs, 2, 40-59 (2000).

In addition to the protein kinases identified above, many other protein kinases have been considered to be therapeutic targets, and numerous publications disclose inhibitors of kinase activity, as reviewed in the following: McMahon et al., Curr. Opin. Drug Disc. Dev., 1, 131-146 (1998), Strawn et al., Exp. Opin. Invest. Drugs, 7, 553-573 (1998), Adams et al., Curr. Opin. Drug Disc. Dev., 2, 96-109 (1999), Stover et al., Curr. Opin. Drug Disc. Dev., 2, 274-285 (1999), Toledo et al., Curr. Med. Chem., 6, 775-805 (1999), and Garcia-Echeverria, et al., Med. Res. Rev., 20, 28-57 (2000).

There is still a need, however, for more potent inhibitors of protein kinases. Moreover, as is understood by those skilled in the art, it is desirable for kinase inhibitors to possess both high affinity for the target kinase as well as high selectivity versus other protein kinases.

Among others, the following patent publications disclose thiazole compounds: WIPO International Publication No. WO 99/21845 discloses 2,4-diaminothiazoles as CDK inhibitors; WO 99/62890 teaches isothiazoles as anticancer agents; WO 98/04536 describes thiazoles as protein kinase C inhibitors; EP 816362A(1998) discloses thiazoles as principally for dopamino D4 receptor antagonists. Aminothiazoles were reported in WO 99/65844 and WO 99/24416, and aminobenzothiazoles in WO 99/24035. WO 00/17175 describes other aminothiazoles as p38 mitogen-activated protein (MAP) kinase inhibitors, and WO 00/26202, WO 00/26203, and U.S. Pat. No. 6,114,365 describe aminothiazoles and ureidothiazoles as anti-tumor agents.

WIPO International Publication No. WO 99/21845 teaches 4-aminothiazole derivatives containing a substituted aryls or heteroaryls. The present invention is based on the discovery that thiazole compounds with 2-amino group substituted with N-containing cycloalkyl often show surprisingly higher activity against protein kinases and more potent cell growth inhibition over the known compounds. Thus, the inventive compounds often show more potent cell growth inhibition.

### SUMMARY OF THE INVENTION

Accordingly, an objective of the invention is to discover potent anti-proliferative agents. Another objective of the invention is to discover effective inhibitors of protein kinases.

These and other objectives of the invention, which will become apparent from the following description, have been achieved by the discovery of the 4-aminothiazole compounds with 2-amino group substituted with N-containing cycloalkyl, pharmaceutically acceptable prodrugs, pharmaceutically active metabolites, and pharmaceutically acceptable salts thereof (such compounds, prodrugs, metabolites and salts are collectively referred to as “agents”) described below, that modulate and/or inhibit cell growth.

Thus, the inventive agents and pharmaceutical compositions containing such agents are expected to be useful in treating various diseases or disorder states associated with uncontrolled or unwanted cellular proliferation such as cancer, autoimmune diseases, viral diseases, fungal diseases, neurodegenerative disorders and cardiovascular diseases.

Further, the agents modulate and/or inhibit the activity of protein kinases, for example one or more CDKs such as CDK2, CDK4 and/or CDK6, or cyclin complexes thereof, and/or one or more LCKs, VEGF or FGFs. Thus, the pharmaceutical compositions containing such agents are useful in treating diseases mediated by kinase activity, such as cancer.

In a general aspect, the invention is directed to a compound or a pharmaceutically acceptable salt represented by Formula (I): 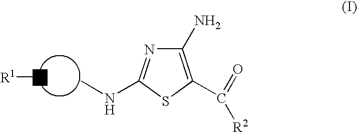
wherein:

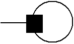

is a nitrogen-containing 3- to 10-membered heterocyclyl ring optionally substituted by one to three substituents selected from R⁷;
R¹ is:
   i) R⁴;
   ii) a group having a formula —SOₙ-T-(CR⁹R¹⁰)_{b}R³, —SOₙ—(CR⁹R¹⁰)_{b}-T-R³, —SOₙNR⁴C(O)R³, wherein n or b are, independently, 0, 1 or 2 and T is a bond, —O—, —NR⁴—, or —S—; or

iii) a group having a formula —C(═O)—R³, —C(═O)—HC═CH—R³, —C(═O)NHR³, —C(═O)NR⁵R⁶ or —C(═S)R³;
R² is (C₁-C₈)alkyl, (C₃-C₁₀)cycloalkyl, —O—(C₁-C₈)alkyl, (C₆-C₁₀)aryl, or 4- to 10-membered heterocyclyl, optionally substituted by one to four substituents selected from R⁷;

wherein R³ is OH, F, Cl, Br, I, CN, CF₃, NO₂, —NR⁵R⁶, —O—R⁴, —SOₚ—R⁴ wherein p is 0, 1, or 2, —POₚ—R⁴ wherein p is 3 or 4, (C₁-C₈)alkyl, —(CH₂)_{d}(C₃-C₁₃)cycloalkyl, —O—(C₁-C₈)alkyl, —(CH₂)_{d}—(C₆-C₁₀)aryl, —(CH₂)_{d}-(4- to 10-membered heterocyclyl), (C₂-C₆)alkenyl, (C₂-C₆)alkynyl, —SO_{q}—NR⁵R⁶, wherein d is an intenger 0 to 6 and q is 1 or 2, —C(═O)—R⁸, —C(O)OR⁸, or —C(═O)—NR⁵R⁶;

wherein R⁴ is each independently selected from the group consisting of hydrogen, (C₁-C₈)alkyl, (C₂-C₆)alkenyl, (C₂-C₆)alkynyl, —O—(C₁-C₈)alkyl, —(CH₂ₑ—(C₃-C₁₃)cycloalkyl, —(CH₂)ₑ—(C₆-C₁₀)aryl, or —(CH₂)ₑ-(4- to 10-membered heterocyclyl);

wherein R⁵ is independently H or (C₁-C₈)alkyl;

wherein R⁶ is selected from the group consisting of —Si(CH₃)₃, (C₁-C₈)alkyl, —O—(C₁-C₈)alkyl, —CH₂—(C═O)—O—(C₁-C₈)alkyl, (C₃-C₁₀)cycloalkyl, (C₆-C₁₀)aryl, and 4- to 10-membered heterocyclyl; or R⁵ and R⁶ when attached to the same nitrogen may optionally be taken together with the same nitrogen to form a 5- to 10-membered heterocyclyl ring;

wherein each (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl, in the above definitions of said R³, R⁴, R⁵, R⁶ and R⁸ may be optionally substituted by one to four R⁷ substituents;

wherein R⁷ is (C₁-C₈)alkyl, (C₃-C₁₃)cycloalkyl, (C₆-C₁₀)aryl, 4- to 10-membered heterocyclyl, (C₂-C₆) alkenyl, (C₂-C₆) alkynyl, —O—(C₁-C₈)alkyl, H, OH, F, Cl, Br, I, CN, CF₃, amidino, —C(O)OR⁹, —C(O)R⁹, —SR⁹, —SO₂R⁹, —NO₂, —NR⁹C(O)R¹⁰, —OC(O)R⁹-aryl, —NSO₂R⁹, —SC(O)R⁹, —NC(═S)NR⁹R¹⁰, —O—N═CR⁹, —N═N—R⁹, —C(O)NR⁹R¹⁰, —(CH₂)ₜ—NR⁹R¹⁰, 2- to 10-membered heteroalkyl, 3- to 10-membered heteroalkenyl, 3- to 10-membered heteroalkynyl, —(CH₂)ₜ(C₆-C₁₀ aryl), —(CH₂)ₜ(4 to 10 membered heterocyclic), -(2 to 10 membered heteroalkyl)-(C₆-C₁₀ aryl), -(2 to 10 membered heteroalkyl)-(4 to 10 membered heterocyclyl), —(CH₂)ₜO(CH₂)ᵤOR⁹, and —(CH₂)ₜOR⁹, wherein t is an integer from 0 to 6 and u is an integer from 2 to 6, H or (C₁-C₈)alkyl;

wherein R⁸ is selected from the group consisting of H, OH, CF₃, (C₁-C₈)alkyl, (C₂-C₆)alkenyl, (C₂-C₆)alkynyl, —O—(C₁-C₈)alkyl, (C₃-C₁₀)cycloalkyl, —O—(C₃-C₁₀)cycloalkyl, 4- to 10-membered heterocyclyl, and 4- to 10-membered —O-heterocyclyl;

wherein each R⁹ and R¹⁰ are independently selected from the group consisting of H, (C₁-C₈)alkyl, (C₁-C₈)alkoxyl, —CH₂—(C═O)—O—(C₁-C₈)alkyl, (C₃-C₁₀)cycloalkyl, (C₆-C₁₀)aryl, and 4- to 10-membered heterocyclyl; or R⁹ and R¹⁰ when together attached to the same N, may optionally be taken together with the same nitrogen to form a 5- to 10-membered heterocyclyl ring; with the proviso that where R⁹ and R¹⁰ are both attached to the same nitrogen, then R⁹ and R¹⁰ are not both bonded to the nitrogen directly through an oxygen;

wherein any of the ring members of each (C₃-C₁₃)cycloalkyl or 4- to 10-membered heterocyclyl in R³, R⁴, R⁶, R⁷, R⁸, R⁹ and R¹⁰ may be optionally substituted with an oxo (═O) and wherein any of the (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl in R⁷, R⁹ and R¹⁰ may be independently further substituted with at least one OH, F, CL, Br, I, CN, CF₃, NO₂, —(C₁-C₈)alkyl, —(C₁-C₈) alkoxyl, COH, or C(O)—(C₁-C₈alkyl).

In one embodiment, the invention is directed to a compound or salt wherein R¹ is R⁴, optionally substituted by one or more R⁹ substituents.

In another embodiment, the invention is directed to a compound or pharmaceutically acceptable salt wherein R¹ is a group having a formula —SOₙ-T-(CR⁹R¹⁰)_{b}R³, —SOₙ—(CR⁹R¹⁰)_{b}-T-R³, —SOₙNR⁴C(O)R³, wherein n or b are, independently, 0, 1 or 2 and T is a bond, —O—, —NR⁴—, or —S—. In a further aspect of this embodiment, wherein R¹ is —SOₙ-T-R³, T is as defined above and R³ is a 4- to 10-membered heterocyclic, optionally substituted by one to four substituents selected from R⁷. In a still further aspect of this embodiment, T is a bond, R³ is a 4- to 10-membered heterocyclic and R⁷ is an —(C₁-C₈)alkyl. In an alternative aspect of this embodiment, T is a bond, R³ is a 5-membered heterocyclyl; and R⁷ is (C₁-C₈)alkyl, (C₃-C₁₃)cycloalkyl, (C₆-C₁₀)aryl, or 4- to 10-membered heterocyclyl, —O—(C₁-C₈)alkyl, (C₂-C₆)alkenyl, or (C₂-C₆)alkynyl; wherein each (C₁-C₈)alkyl, (C₃-C₁₃)cycloalkyl, (C₆-C₁₀)aryl, or 4- to 10-membered heterocyclyl, —O—(C₁-C₈)alkyl, (C₂-C₆)alkenyl, or (C₂-C₆)alkynyl may be independently optionally substituted with at least one OH, F, CL, Br, I, CN, CF₃, NO₂, —(C₁-C₈)alkyl, —(C₁-C₈) alkoxyl, COH, or C(O)—(C₁-C₈alkyl). In an alternative aspect of this embodiment, the invention is directed to a compound or salt according to claim 3, wherein the group: 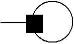
is a nitrogen-containing 4-6 membered heterocyclyl ring optionally substituted with (C₁-C₈)alkyl, (C₃-C₁₀)cycloalkyl, (C₆-C₁₀)aryl, or 4- to 10-membered heterocyclyl; and R² is a (C₆-C₁₀)aryl, or a 4- to 10-membered heterocyclyl having one or more substituents selected from the group consisting of a F, Cl, Br, I.

In another embodiment, the invention is directed to a compound or pharmaceutically acceptable salt represented by Formula (I): 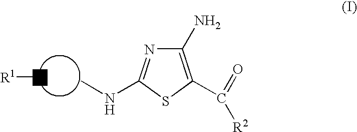
wherein: 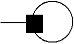
is a nitrogen-containing 3- to 10-membered heterocyclyl ring optionally substituted by one to three substituents selected from R⁷;

wherein R¹ is a group having a formula —C(═O)—R³ —C(═O)—HC═CH—R³, —C(═O)NHR³, —C(═O)NR⁵R⁶ or —C(═S)R₃. In a further aspect of this embodiment, R³ is a —(CH₂)_{d}(C₃-C₁₃)cycloalkyl, —O—(C₁-C₈)alkyl, —(CH₂)_{d}—(C₆-C₁₀)aryl, —(CH₂)_{d}-(4- to 10-membered heterocyclyl), wherein each R³ (C₃-C₁₀)cycloalkyl, (C₆-C₁₀)aryl, or 4- to 10-membered heterocyclic may be optionally substituted by one to four R⁷substituents.

In a still further aspect of this embodiment, wherein R³is a 5-membered heteroaryl; and R⁷is (C₁-C₈)alkyl, (C₃-C₁₀)cycloalkyl, (C₆-C₁₀)aryl, or 4- to 10-membered heterocyclyl, —O—(C₁-C₈)alkyl, (C₂-C₆)alkenyl, or (C₂-C₆)alkynyl; wherein each (C₁-C₈)alkyl, (C₃-C₁₀)cycloalkyl, (C₆-C₁₀)aryl, or 4- to 10-membered heterocyclyl, (C₁-C₈)alkyl-O—, (C₂-C₆)alkenyl, or (C₂-C₆)alkynyl may be optionally substituted with at least one OH, F, CL, Br, I, CN, CF₃, NO₂, —(C₁-C₈)alkyl, —(C₁-C₈) alkoxyl, COH, or C(O)—(C₁-C₈alkyl);

In still another embodiment of this invention, wherein R² is a 4- to 10-membered heterocyclyl having one or more substituents selected from the group consisting of F, Cl, Br, I.

In still a further aspect of this invention, the group: 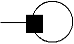
is a nitrogen-containing 4-6 membered heterocyclyl ring optionally substituted by (C₁-C₈)alkyl, (C₃-C₁₀)cycloalkyl, (C₆-C₁₀)aryl, or 4- to 10-membered heterocyclyl; and R² is a (C₆-C₁₀)aryl or 4- to 10-membered heterocyclyl having one or more substituents selected from the group consisting of F, Cl, Br, I.

In another embodiment, the present invention comprises a pharmaceutical composition comprising an amount of active agent effective to modulate cellular proliferation and a pharmaceutically acceptable carrier, said active agent being selected from the group consisting of a compound, or a pharmaceutically acceptable prodrug, pharmaceutically active metabolite, and pharmaceutically acceptable salt thereof.

In another embodiment, the present invention comprises a pharmaceutical composition comprising an amount of active agent effective to inhibit protein kinases and a pharmaceutically acceptable carrier, said active agent being selected from the group consisting of a compound, or a pharmaceutically acceptable prodrug, pharmaceutically active metabolite, and pharmaceutically acceptable salt thereof.

In another embodiment, the present invention comprises a pharmaceutical composition, wherein said protein kinases are selected from CDK1, CDK1/cyclin complex, CDK2, CD K2/cyclin complex, CDK4, CDK4/cyclin complex, CDK6, or CDK6/cyclin complex.

In another embodiment, the present invention comprises a method of treating a disease condition or disorder in association with uncontrolled cellular proliferation, comprising administering to a subject in need thereof a therapeutically effective amount of a compound, or a pharmaceutically acceptable prodrug, pharmaceutically active metabolite, or pharmaceutically acceptable salt thereof.

In another embodiment, the present invention comprises a method of treating a disease condition or disorder, wherein the disease condition or disorder is a tumor growth, angiogenesis, viral infection, autoimmune disease or neurodegenerative disorder.

In another embodiment, the present invention comprises a method of modulating or inhibiting the activity of a protein kinase receptor, comprising delivering to the protein kinase receptor an effective amount of a compound, or a pharmaceutically acceptable prodrug, pharmaceutically active metabolite, or pharmaceutically acceptable salt thereof.

In another embodiment, the present invention comprises a method, wherein the protein kinase receptor is a CDK complex.

In a more preferable aspect, compounds selected from the group consisting of: 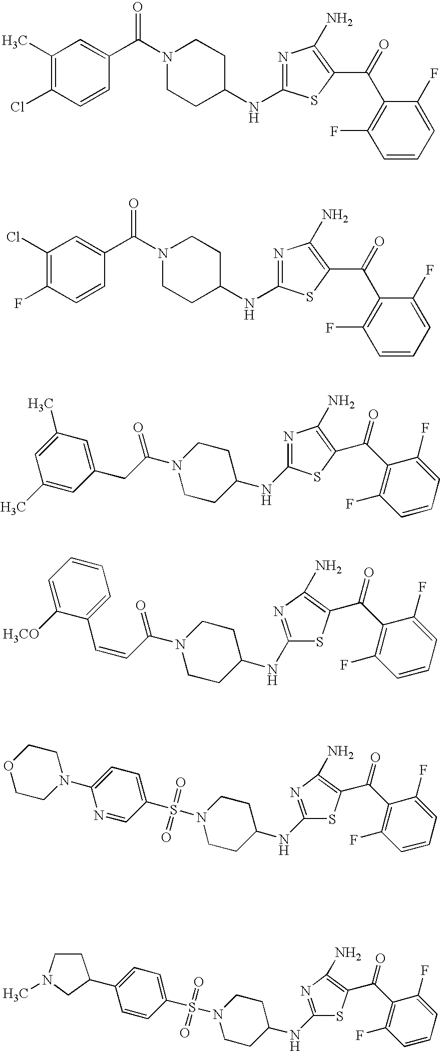 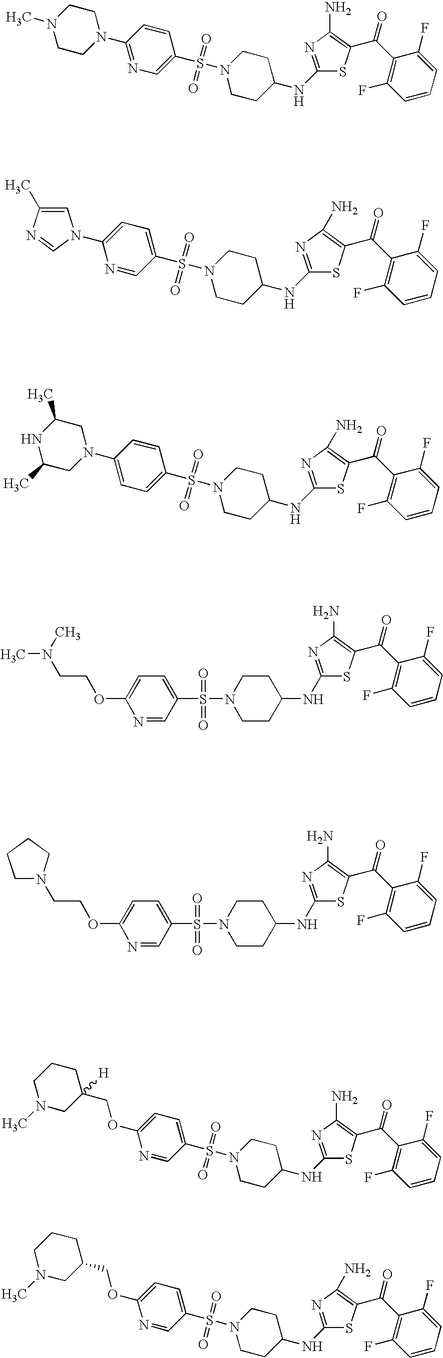 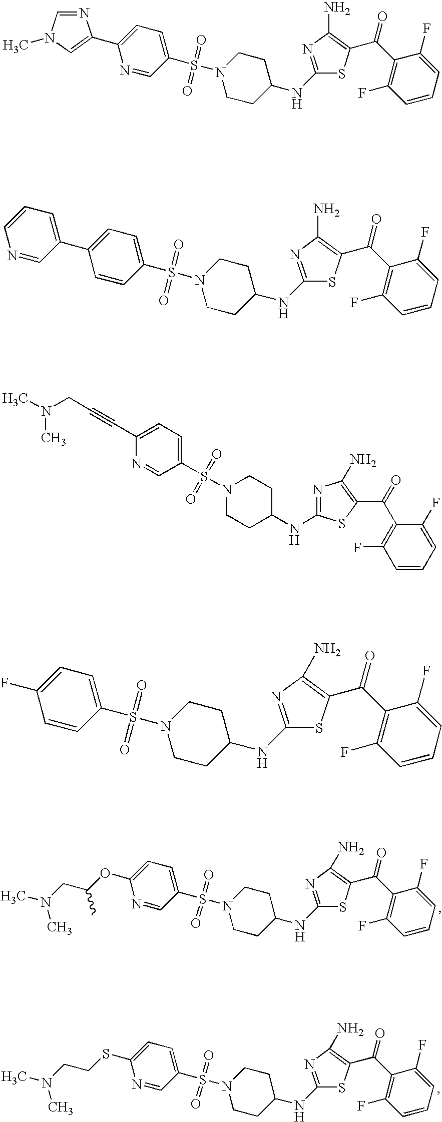 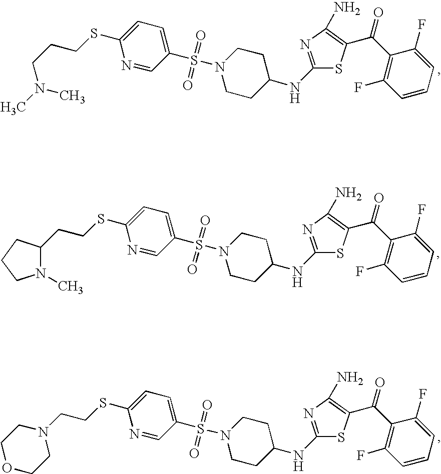
and a pharmaceutically acceptable prodrug thereof, pharmaceutically active metabolite thereof, or pharmaceutically acceptable salt of such compound or metabolite.

The invention also relates to a method of treating proliferative diseases such as cancer, autoimmune diseases, viral diseases, fungal diseases, neurodegenerative disorders and cardiovascular disease, comprising administering effective amounts of a compound of Formula (I) or a pharmaceutically acceptable salt, pharmaceutically acceptable prodrug, pharmaceutically active metabolite, or pharmaceutically acceptable salt of such compound or metabolite to a subject in need of such treatment.

The invention further relates to a method of modulating and/or inhibiting the kinase activity of one or more CDKs such as CDK1, CDK2, CDK4, and/or CDK6 or cyclin complexes thereof, VEGF, FGF and/or LCK by administering a compound of Formula (I) or a pharmaceutically acceptable salt, pharmaceutically acceptable prodrug, or pharmaceutically acceptable salt of such compound or metabolite thereof.

The invention also relates to pharmaceutical compositions, each comprising an effective amount of an agent selected from compounds of Formula (I) and pharmaceutically active metabolites, pharmaceutically acceptable prodrugs, and pharmaceutically acceptable salts of such compounds and metabolites, and a pharmaceutically acceptable carrier or vehicle for such agent.

The inventive compounds of Formula (I) are potent anti-proliferative agents. The compounds are also useful for mediating the activity of protein kinases. More particularly, the compounds are useful as agents for modulating and/or inhibiting the activity of various enzymes, for example protein kinases, thus providing treatments for cancer or other diseases associated with uncontrolled or abnormal cellular proliferation.

The diseases or disorders in association with uncontrolled or abnormal cellular proliferation include, but are not limited to, the following:
a variety of cancers, including, but not limited to, carcinoma, hematopoietic tumors of lymphoid lineage, hematopoietic tumors of myeloid lineage, tumors of mesenchymal origin, tumors of the central and peripheral nervous system and other tumors including melanoma, seminoma and Kaposi's sarcoma and the like.
a disease process which features abnormal cellular proliferation, e.g., benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.
defective apoptosis-associated conditions, such as cancers (including but not limited to those types mentioned hereinabove), viral infections (including but not limited to herpesvirus, poxvirus, Epstein-Barr virus, Sindbis virus and adenovirus), prevention of AIDS development in HIV-infected individuals, autoimmune diseases (including but not limited to systemic lupus erythematosus, rheumatoid arthritis, psoriasis, autoimmune mediated glomerulonephritis, inflammatory bowel disease and autoimmune diabetes mellitus), neurodegenerative disorders (including but not limited to Alzheimer's disease, amyotrophic lateral sclerosis, retinitis pigmentosa, Parkinson's disease, AIDS-related dementia, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteroporosis and arthritis), aspirin-sensitive rhinosinusitis, cystic fibrosis, multiple sclerosis, kidney diseases and cancer pain.

The active agents of the invention may also be useful in the inhibition of the development of invasive cancer, tumor angiogenesis and metastasis.

Moreover, the active agents of the invention, as inhibitors of the CDKs, can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like.

Several terms employed throughout the present application are described below.

The terms “comprising” and “including” are used herein in their open, non-limiting sense.

The terms “comprising” and “including” are used herein in their open, non-limiting sense.

The terms “abnormal cell growth” and “hyperproliferative disorder” are used interchangeably in this application.

“Abnormal cell growth”, as used herein, refers to cell growth that is independent of normal regulatory mechanisms (e.g., loss of contact inhibition), including the abnormal growth of normal cells and the growth of abnormal cells. This includes, but is not limited to, the abnormal growth of: (1) tumor cells (tumors), both benign and malignant, expressing an activated Ras oncogene; (2) tumor cells, both benign and malignant, in which the Ras protein is activated as a result of oncogenic mutation in another gene; (3) benign and malignant cells of other proliferative diseases in which aberrant Ras activation occurs. Examples of such benign proliferative diseases are psoriasis, benign prostatic hypertrophy, human papilloma virus (HPV), and restinosis. “Abnormal cell growth” also refers to and includes the abnormal growth of cells, both benign and malignant, resulting from activity of the enzyme farnesyl protein transferase.

The term “treating”, as used herein, unless otherwise indicated, means reversing, alleviating, inhibiting the progress of, or preventing the disorder or condition to which such term applies, or one or more symptoms of such disorder or condition. “Treating” is intended to mean at least the mitigation of a disease condition in a subject such as mammal (e.g., human), that is affected, at least in part, by the activity of one or more kinases, for example protein kinases such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition. The term “treatment”, as used herein, refers to the act of treating, as “treating” is defined immediately above.

The term “halo”, as used herein, unless otherwise indicated, means fluoro, chloro, bromo or iodo. Preferred halo groups are fluoro, chloro and bromo.

The term “alkyl”, as used herein, unless otherwise indicated, means saturated monovalent hydrocarbon radicals having straight, cyclic or branched moieties. Said “alkyl” group may include an optional carbon-carbon double or triple bond where said alkyl group comprises at least two carbon atoms. It is understood that for cyclic moieties at least three carbon atoms are required in said alkyl group.

The term “alkoxy”, as used herein, unless otherwise indicated, means 0-alkyl groups wherein “alkyl” is as defined above.

The term “amidino”, as used herein, means —C(═NH)—NH₂.

The term “heteroalkyl” as used herein refers to straight- and branched-chain alkyl groups having from two to ten atoms containing one or more heteroatoms selected from S, O, and N. Illustrative alkyl groups include alkylaminos, aminoalkyl, s-alkyl, o-alkyl, and the like. Correspondingly, the terms “heteroalkenyl” and “heteroalkynyl” refers to straight- and branched-chain alkenyl and alkynyl groups, respectively, having from three to ten atoms containing one or more heteroatoms selected from S, O and N.

The term “alkenyl” refers to straight- and branched-chain alkenyl groups having from two to twelve carbon atoms. Illustrative alkenyl groups include prop-2-enyl, but-2-enyl, but-3-enyl, 2-methylprop-2-enyl, hex-2-enyl, and the like.

The term “alkynyl” refers to straight- and branched-chain alkynyl groups having from two to twelve carbon atoms. Illustrative alkynyl groups include prop-2-ynyl, but-2-ynyl, but-3-ynyl, 2-methylbut-2-ynyl, hex-2-ynyl, and the like.

The term “cycloalkyl” refers to a monocyclic or polycyclic radical which may be saturated or unsaturated and contains carbocycles having from three to twelve carbon atoms, including bicyclic and tricyclic cycloalkyl structures.

A “heterocycloalkyl” group refers to a monocyclic or polycyclic radical which may be saturated or unsaturated and contains from three to twelve ring atoms, selected from carbon and heteroatoms, preferably 4 or 5 ring carbon atoms, and at least one heteroatom selected from nitrogen, oxygen and sulfur.

The term “aryl” as used herein, unless otherwise indicated, means an organic radical derived from an aromatic hydrocarbon by removal of one hydrogen, such as phenyl or naphthyl.

The terms “5 membered heterocyclic”, “5 or 6 membered heterocyclic”, “5 to 8 membered heterocyclic”, “5 to 10 membered heterocyclic” or “5 to 13 membered heterocyclic”, as used herein, unless otherwise indicated, includes aromatic and non-aromatic heterocyclic groups containing one to four heteroatoms each selected from O, S and N, wherein each heterocyclic group has from 5, 6, 5 to 8, 5 to 10 or 5 to 13 atoms, respectively, in its ring system. The heterocyclic groups include benzo-fused ring systems and ring systems substituted with one or two oxo (═O) moieties such as pyrrolidin-2-one. An example of a 5 membered heterocyclic group is thiazolyl, an example of a 10 membered heterocyclic group is quinolinyl, and an example of a 13 membered heterocyclic group is a carbazole group. Examples of non-aromatic heterocyclic groups are pyrrolidinyl, piperidino, morpholino, thiomorpholino and piperazinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl and thiazolyl. Heterocyclic groups having a fused benzene ring include benzimidazolyl, benzofuranyl, and benzo[1,3]dioxolyl.

The term “alcohol” refers to the radical —R—OH where R is alkyl, alkenyl, alkynyl, Ar, heteroaryl, heterocycloalkyl, or cycloalkyl as defined above. Examples of alcohols include methanol, ethanol, propanol, phenol and the like.

The term “acyl” represents —C(O)R, —C(O)OR, —OC(O)R or —OC(O)OR where R is alkyl, alkenyl, alkynyl, Ar, heteroaryl, heterocycloalkyl, or cycloalkyl as defined as above.

The term “amide” refers to the radical —C(O)N(R′)(R″) where R′ R″ are each independently selected from hydrogen, alkyl, alkenyl, alkynyl, —OH, alkoxy, cycloalkyl, heterocycloalkyl, heteroaryl, aryl as defined above; or R′ and R″ cyclize together with the nitrogen to form a heterocycloalkyl or heteroaryl as defined above.

The term “substituted” as used herein means that the group in question, e.g., alkyl group, etc., may bear one or more substituents.

The alkyl, cycloalkyl, aryl, heterocyclyl groups and the substituents containing these groups, as defined hereinabove, may be optionally substituted by at least one other substituent. The term “optionally substituted” is intended to expressly indicate that the specified group is unsubstituted or substituted by one or more substituents from the list above. Various groups may be unsubstituted or substituted (i.e., they are optionally substituted) as indicated.

If the substituents themselves are not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999), which is incorporated herein by reference in its entirety. In some instances, a substituent may be specifically selected to be reactive under the reaction conditions used in the methods of this, invention. Under these circumstances, the reaction conditions convert the selected substituent into another substituent that is either useful in an intermediate compound in the methods of this invention or is a desired substituent in a target compound.

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

The compounds of present invention may in certain instances exist as tautomers. This invention relates to the use of all such tautomers and mixtures thereof.

The term “prodrug”, as used herein, unless otherwise indicated, means compounds that are drug precursors, which following administration, release the drug in vivo via some chemical or physiological process (e.g., a prodrug on being brought to the physiological pH is converted to the desired drug form).

Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula I. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

It will be appreciated that any solvate (e.g. hydrate) form of compounds of formula I and prodrugs thereof can be used for the purpose of the present invention.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, phosphoric acid and the like, or with an organic acid, such as acetic acid, phenylacetic acid, propionic acid, stearic acid, lactic acid, ascorbic acid, maleic acid, hydroxymaleic acid, isethionic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid, 2-acetoxybenzoic acid or cinnamic acid, a sulfonic acid, such as p-toluenesulfonic acid, methanesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine and piperazine, and inorganic salts derived from sodium, calcium, potassium, magnesium, manganese, iron, copper, zinc, aluminum and lithium.

Pharmaceutical compositions according to the invention may, alternatively or in addition to a compound of Formula I, comprise as an active ingredient pharmaceutically acceptable prodrugs, pharmaceutically active metabolites, and pharmaceutically acceptable salts of such compounds and metabolites. Such compounds, prodrugs, multimers, salts, and metabolites are sometimes referred to herein collectively as “active agents” or “agents.”

In the case of agents that are solids, it is understood by those skilled in the art that the inventive compounds and salts may exist in different crystal or polymorphic forms, all of which are intended to be within the scope of the present invention and specified formulas.

Therapeutically effective amounts of the active agents of the invention may be used to treat diseases mediated by modulation or regulation of various kinases, for example protein kinases. An “effective amount” is intended to mean that amount of an agent that significantly inhibits proliferation and/or prevents de-differentiation of a eukaryotic cell, e.g., a mammalian, insect, plant or fungal cell, and is effective for the indicated utility, e.g., specific therapeutic treatment.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated.

Agents that potently regulate, modulate, or inhibit cell proliferation are preferred. For certain mechanisms, inhibition of the protein kinase activity associated with CDK complexes, among others, and those which inhibit angiogenesis and/or inflammation are preferred. The present invention is further directed to methods of modulating or inhibiting protein kinase activity, for example in mammalian tissue, by administering an inventive agent. The activity of agents as anti-proliferatives is easily measured by known methods, for example by using whole cell cultures in an MTT assay. The activity of the inventive agents as modulators of protein kinase activity, such as the activity of kinases, may be measured by any of the methods available to those skilled in the art, including in vivo and/or in vitro assays. Examples of suitable assays for activity measurements include those described in WIPO International Publication No. WO 99/21845; Parast et al., Biochemistry, 37, 16788-16801 (1998); Connell-Crowley and Harpes, Cell Cycle: Materials and Methods, Michele Pagano, ed. Springer, Berlin, Germany (1995); WIPO International Publication No. WO 97/34876; and WIPO International Publication No. WO 96/14843. These properties may be assessed, for example, by using one or more of the biological testing procedures set out in the examples below.

The active agents of the invention may be formulated into pharmaceutical compositions as described below. Pharmaceutical compositions of this invention comprise an effective modulating, regulating, or inhibiting amount of a compound of Formula I and an inert, pharmaceutically acceptable carrier or diluent. In one embodiment of the pharmaceutical compositions, efficacious levels of the inventive agents are provided so as to provide therapeutic benefits involving anti-proliferative ability. By “efficacious levels” is meant levels in which proliferation is inhibited, or controlled. These compositions are prepared in unit-dosage form appropriate for the mode of administration, e.g., parenteral or oral administration.

An inventive agent can be administered in conventional dosage form prepared by combining a therapeutically effective amount of an agent (e.g., a compound of Formula I) as an active ingredient with appropriate pharmaceutical carriers or diluents according to conventional procedures. These procedures may involve mixing, granulating and compressing or dissolving the ingredients as appropriate to the desired preparation.

The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent can be dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease being treated. Optimal dosages for a given set of conditions can be ascertained by those skilled in the art using conventional dosage-determination tests in view of the experimental data for an agent. For oral administration, an exemplary daily dose generally employed is from about 0.001 to about 1000 mg/kg of body weight, with courses of treatment repeated at appropriate intervals. Administration of prodrugs is typically dosed at weight levels which are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active agents in water-soluble form. Additionally, suspensions of the agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/ciliary, lens, choroid/retina and sclera. The pharmaceutically acceptable ophthalmic vehicle may be an ointment, vegetable oil, or an encapsulating material. A compound of the invention may also be injected directly into the vitreous and aqueous humor.

Alternatively, the active agents may be in powder form for constitution with a suitable vehicle, e.g., sterile pyrogen-free water, before use. The compounds may also be formulated in rectal compositions such as suppositories or retention enemas, e.g, containing conventional suppository bases such as cocoa butter or other glycerides.

In addition to the formulations described above, the active agents also can be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

The active agents of the invention may be useful in combination with known anti-cancer treatments such as, but not limited to, DNA interactive agents such as cisplatin or doxorubicin; topoisomerase II inhibitors such as etoposide, topoisomerase I inhibitors such as CPT-11 or topotecan; tubulin interacting agents such as paclitaxel, docetaxel or the epothilones; hormonal agents such as tamoxifen; thymidilate synthase inhibitors such as 5-fluorouracil; and anti-metalbolites such as methotrexate. They may be administered together or sequentially, and when administered sequentially, the inventive agents may be administered either prior to or after administration of the known anticancer or cytotoxic agent.

The inventive agents may be prepared using the reaction routes and synthesis schemes as described below, employing the general techniques known in the art using starting materials that are readily available. The preparation of preferred compounds of the present invention is described in detail in the following examples, but the artisan will recognize that the chemical reactions described may be readily adapted to prepare a number of other anti-proliferatives or protein kinase inhibitors of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### DETAILED DESCRIPTION OF THE INVENTION

### EXAMPLES

In the examples described below, unless otherwise indicated, all temperatures are set forth in degrees Celsius and all parts and percentages are by weight. Reagents were purchased from commercial suppliers such as Sigma-Aldrich Chemical Company or Lancaster Synthesis Ltd. and were used without further purification unless otherwise indicated. Tetrahydrofuran (THF) and N,N-dimethylformamide (DMF) were purchased from Aldrich in Sure Seal bottles and used as received. All solvents were purified using standard methods known to those skilled in the art, unless otherwise indicated.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates from Analtech (0.25 mm), eluted with the appropriate solvent ratios (v/v), and are denoted where appropriate. The reactions were assayed by TLC, NMR, or analytical HPLC and terminated as judged by the consumption of starting material.

Visualization of the TLC plates was done with iodine vapor, ultraviolet illumination, 2% Ce(NH₄)₄(SO₄)₄ in 20% aqueous sulfuric acid, 2% ninhydrin in ethanol, or p-anisaldehyde spray reagent, and activated with heat where appropriate. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or MgSO₄ prior to filtration and evaporation of the solvents under reduced pressure on a rotary evaporator and noted as solvents removed in vacuo. Hydrogenolysis was done at the pressure indicated in the examples or at ambient pressure. Flash column chromatography (Still et al., J. Org. Chem., 43, 2923 (1978)) was done using Merck silica gel (47-61 μm) with a silica gel crude material ratio of about 20:1 to 50:1, unless otherwise stated.

Reversed phase preparative HPLC purification was performed on Gilson 321 system, using a C18-reversed phase preparative column (Metasil AQ 10μ, C18, 120A 250×21.2 mm, MetaChem), and eluted with a gradient from 0.1% TFA/5% CH₃CN/H₂O to 0.1% TFA/5% H₂O/CH₃CN over 20 minutes at a flow rate of 20 ml/min.

For these typically basic compounds, free bases were obtained upon concentration of HPLC fractions, dissolution in ethyl acetate, neutralization upon washing with aqueous Na₂CO₃, and evaporation in vacuo. For the corresponding trifluoroacetic acid (TFA) salts, TFA was present in the eluant, thus no treatment was necessary, and HPLC fractions were directly lyophilized or concentrated in vacuo. For the corresponding HCl salts, excess aqueous hydrochloric acid was added to enriched HPLC fractions prior to lyophilization or concentration under reduced pressure, unless other procedures were used as otherwise indicated.

¹H-NMR spectra were recorded on a Bruker or Varian instrument operating at 300 MHz and ¹³C-NMR spectra were recorded operating at 75 MHz. NMR spectra were obtained as CDCl₃ solutions (reported in ppm), using chloroform as the reference standard (7.27 ppm and 77.00 ppm) unless otherwise noted. When peak multiplicities are reported, the following abbreviations are used: s (singlet), d (doublet), t (triplet), q (quartet), m (multiplet), br (broadened multiplet), bs (broadened singlet), dd (doublet of doublets), dt (doublet of triplets). Coupling constants, when given, are reported in Hertz (Hz).

Infrared (IR) spectra were recorded on a Perkin-Elmer FT-IR Spectrometer as neat oils, as KBr pellets, or as CDCl₃ solutions, and when given are reported in wave numbers (cm⁻¹). The mass spectra were obtained using LSIMS, FAB, MALDI, or electrospray (ESIMS). All melting points (mp) are uncorrected.

Mass spectrometry (MS) was conducted with various techniques. Matrix-Assisted Laser Desorption/Ionization Fourier Transform Mass Spectrometry (MALDI FTMS), was performed on an IonSpec FTMS mass spectrometer. Samples are irradiated with a nitrogen laser (Laser Science Inc.) operated at 337nm and the laser beam is attenuated by a variable attenuator and focused on the sample target. The ions are then differentiated according to their m/z using an ion cyclotron resonance mass analyzer. The electrospray ionization (ESI) mass spectrometry experiments were performed on an API 100 Perkin Elmer SCIEX single quadrupole mass spectrometer. Electrospray samples are typically introduced into the mass analyzer at a rate of 4.0 μl/minute. The positive and negative ions, generated by charged droplet evaporation, enter the analyzer through an interface plate and a 100 mm orifice, while the declustering potential is maintained between 50 and 200V to control the collisional energy of the ions entering the mass analyzer. The emitter voltage is typically maintained at 4000V. The liquid chromatography (LC) electrospray ionization (ESI) mass spectrometry experiments are performed on a Hewlett-Packard (HP) 1100 MSD single quadrupole mass spectrometer. Electrospray samples are typically introduced into the mass analyzer at a rate of 100 to 1000 μl/minute. The positive and negative ions, generated by charged droplet evaporation, enter the analyzer through a heated capillary plate, while the declustering potential is maintained between 100 and 300V to control the collisional energy of the ions entering the mass analyzer. The emitter voltage is typically maintained at 4000V.

Compounds in accordance with the invention may be prepared in manners analogous to those specifically described below, with the lettered example prefixes (i.e., A, B, C, D, E, F, G, H, I, J, K, L, M, N, O and P) designating general synthesis schemes.

General routes to the compounds of the invention are described as follows. In these Schemes and its explanations, R¹ through R¹⁹ have the same meanings as defined above, unless indicated otherwise.

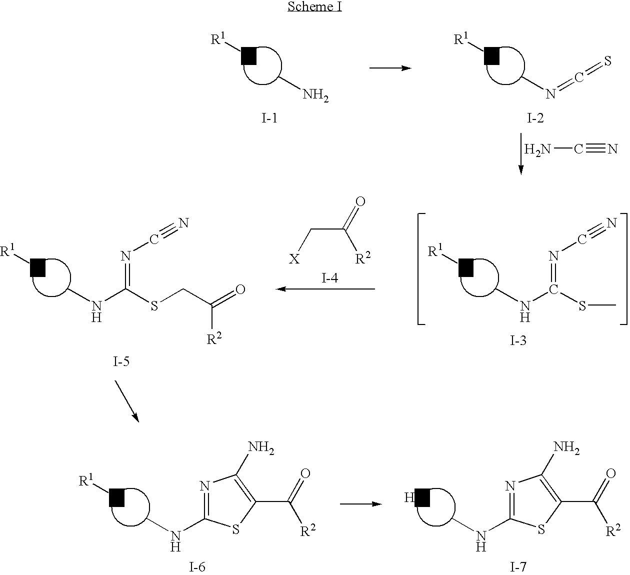

Amino-substituted cycloalkylamines, represented as I-1 in the route labeled Scheme I, are converted in any of numerous standard methods to their corresponding isothiocyanates I-2, typically with thiophosgene, under acidic, basic or neutral conditions, depending on the particular R¹ in substrate I-1. The isothiocyanate I-2 is a typical reaction partner in a routine 2,4-diaminothiazole construction (see World Patent Application WO 99/21845 and Gewald, et al., J. Prakt. Chem., 35, 97-104 (1967)). Condensation of cyanamide with isothiocyanate I-2 in the presence of a strong, but hindered tertiary base such as 1,8-diazabicyclo[5.4.0]undec-7-ene (DBU) or triethylamine (Et₃N) provides the isothiourea anion I-3, which is S-alkylated in situ with a halocarbonyl I-4 to transitory intermediate I-5. Many different halocarbonyl I-4, particularly poly-substituted acetophenones are used, including examples from World Patent Application WO 99/21845, and additional preparations herein. Base-promoted enolization of isothiourea I-5 causes cyclization to furnish diaminothiazole I-6. When the R¹ in I-6 is a routine nitrogen protecting group, such as a t-butoxycarbonyl, facile deprotection is produced with standard methods, i.e. trifluoroacetic acid or hydrogen chloride in dioxane, to provide a key, pivotal, late stage, intermediate amine I-7, which was further elaborated in many ways. Of course Scheme 1 may be employed with any R¹ group that incorporates the targeted functionality, as long as R¹ is a moiety that may withstand the alkaline conditions.

The starting material I-1 for Scheme I are available commercially in many cases, but had to be prepared for selected examples herein, as shown in Scheme II below. Many cycloalkylamino-ketones II-1 were purchasable, for example N-t-butoxycarbonyl-4-piperidone, or prepared according to literature (e.g., see U.S. Pat. No. 5,968,929). The ketones II-1 could be transformed via routine reductive amination methods directly to amines l-1, but a convenient intermediate was oxime II-2, which could be reduced with Raney nickel under hydrogen atmosphere or typical hydride reagents, such as lithium aluminum hydride (e.g., see U.S. Pat. No. 5,968,929). Alternatively, many alcohols II-3 are available from literature or commercial suppliers, and II-3 could be processed as precedented in the literature, for example as the corresponding sulfate esters II-4 (i.e. mesylates or tosylates). The sulfate esters I-4 or equivalent are converted to the azides II-5, which are easily reduced to the desired amines I-1 with standard protocols.

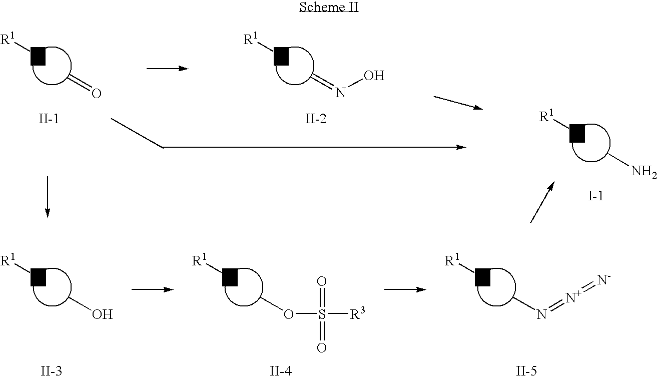

With a free amine available on a cycloalkylamino-diaminothiazole template such as I-7 from Scheme I, numerous nitrogen-capped derivatives are available from the use of various reagents, some of which are outlined in the scheme labeled Scheme III below. For example, isocyanates Ill-1 give ureas III-2. Activated esters, mostly as acyl chlorides III-3, provide amides (III-4, R⁵=alkyl), urethanes (III-4, R⁵=alkoxy), or thiocarbamate (III-4, R⁵=alkylthio) from acid chlorides (III-3, R⁵=alkyl), chloroformates (III-3, R⁵=alkoxy), or chlorothioformates (III-3, R⁵=alkylthio), respectively. Another avenue to amides (III-5, R⁶=alkyl) was available from coupling of carboxylic acids (III-5R⁶=alkyl) to amine I-7 with any of a variety of peptide coupling reagents, such as benzotriazol-1-yloxytris(pyrrolidino)-phosphonium hexafluorophosphate (PyBOP) or O-(7-azabenzotriazol-1-yl)-N,N,N′,N′-tetramethyluronium hexafluorophosphate (HATU). Halosulfonyl reagents III-7 are also good reactants to afford sulfonamides III-8 (R⁷=alkyl) or sulfamides III-8 (R⁷=alkylamino) from sulfonyl chlorides/fluorides (III-7, R⁷=alkyl, X=Cl or F) or sulfamyl chlorides (III-7, R⁷=alkylamino, X=Cl). Reductive amination of I-7 with aldehydes III-9 provides N-alkyl derivatives III-10 (R⁸=alkyl). All of the reactions depicted in Scheme III are compatible with parallel, combinatorial methods, and the amines I-7 are very suitable as templates, or core building blocks.

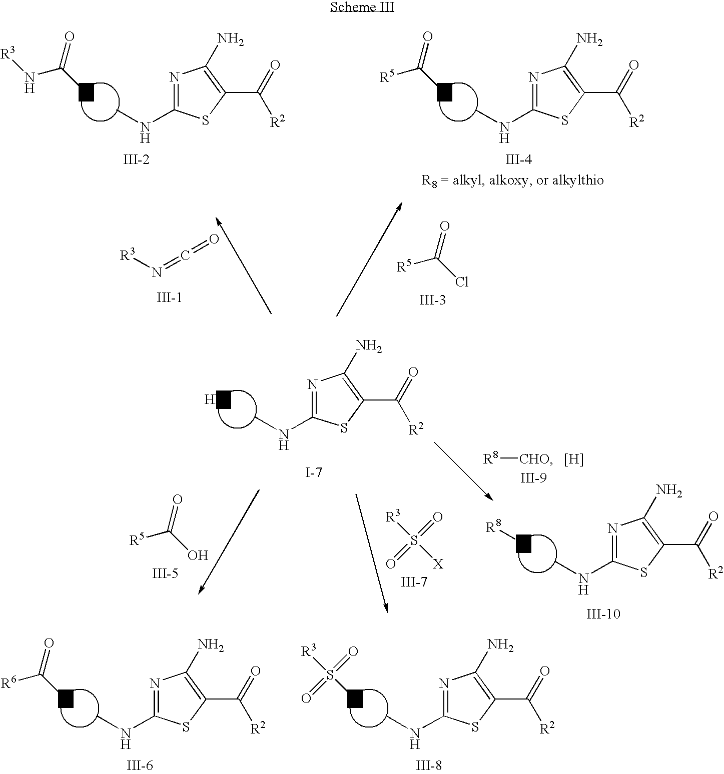

Most of the various reactants for amines I-7 in Scheme III are commercially available, but some sulfonyl chlorides III-7 (R⁷=aryl or heteroaryl) required special preparations, as outlined in Scheme IV. For example, for more highly functionalized arylsulfonyl chlorides IV-2, some traditional methods were applicable. Arylthiols IV-1 could be oxidized to sulfonyl chlorides IV-2 with chlorine gas bubbled through acetic acid solutions. Or substituted aryls IV-3 underwent electrophilic sulfonation with chlorosulfonic acid to produce sulfonic acids IV-4, which can be purified and are mildly converted with phosphorus pentachloride or thionyl chloride to desired sulfonyl chlorides IV-2. In the particular cases for pyridyl-sulfonyl chlorides IV-2 (W=N; U,V=CH), there are many examples from the literature wherein nitropyridine IV-5 (W=N, U,V=C) serves as starting material. The nitro group of IV-5 is reduced to the corresponding amine, which in turn is converted in situ to a diazonium intermediate and substituted with a sulfur nucleophile, such as sulfur dioxide, to sulfonate IV-4, or directly to sulfonyl chloride IV-2 (for an example of this sequence, see Markley, et al, J. Med. Chem., 29, 427-433 (1986)). For pyrimidine sulfonyl chlorides (IV-2, V,W=N; U=CH), Caldwell, et al., J. Amer. Chem. Soc., 81, 5166-5167 (1959) describes the preparation of 2-chloro-pyrimidine-5-sulfonyl chloride from 2-amino-pyrimidine and fuming sulfuric acid. The pyrazine sulfonyl chloride (IV-2, U,W=N; V=CH) should be available via one of the outlined approaches.

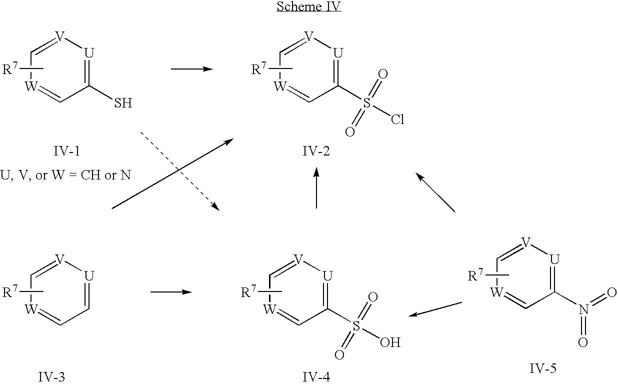

A significant subset of the sulfonamides III-8 (R₉=aryl) were made by elaboration subsequent to the process in Scheme III, via substitution of 2-haloaryl V-1, as shown in Scheme V. Particularly for 2-chloroheteroaryls V-1 (X=Cl), substitution by amines, alcohols, or alkylthiols, was effective, especially when in excess or sometimes as the solvent, in the presence of a base, such as potassium carbonate, at elevated temperature, or as promoted by microwave exposure—to result in 2-substituted pyridines, pyrimidines, or pyrazines. 2-Alkoxy-aryls or heteroaryls V-2 (Z=alkoxy), 2-alkylamino-V-2 (Z=alkylamino), or 2-alkylthio-V-2 (Z=alkylthio), respectively, were obtained in this manner. Similarly some fluorophenyls V-1 (U, V, W=C, X=F) were also susceptible to substitution by alcohols or amines to allow access to certain alkoxy-aryls V-2 (Z=alkoxy, U, V, W=C) or alkylamino-aryls V-2 (Z=alkylamino, U, V, W=C), respectively. 2-Alkyl- or 2-aryl-moieties were attached to either phenyls V-1 (U, V, W=C, X=Br or I) or heteroaryls V-1 (one or two of U,V, or W=N with others C, X ═Cl) to furnish coupled products V-2 (Z=alkenyl, aryl, heteroaryl, or alkynyl), via standard Heck, Stille, Suzuki, or Castro-Stevens coupling methodology, in polar solvent in the presence of catalyst, such as tetrakis(triphenylphosphino)palladium(0), or dichloro-bis(triphenylphosphino)-palladium(II), sometimes with heating, with a suitable coupling partner, such as 3-pyridylboronic acid.

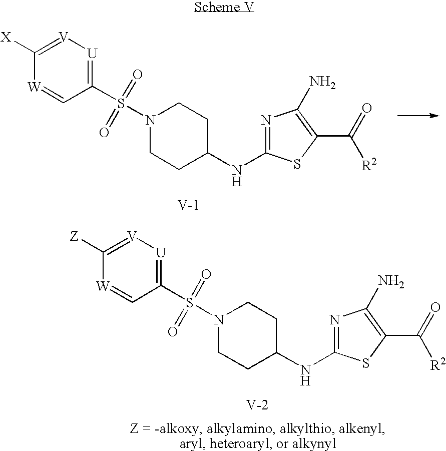

Other processing subsequent to Scheme 3, but upon substituents of aryl or heteroaryl sulfonamides, are exemplified in the following Schemes VI, VII, VIII, IX, and X below. The benzaldehyde VI-1 underwent reductive amination to amines VI-3 under routine conditions, either with hydride reducing agents such as sodium cyanoborohydride, or hydrogenation. One aldehyde VI-1 was made via Scheme III from commercially available sulfonyl chloride III-7 (R⁷=p-C₆H₄—CHO). Aldehydes are also good starting materials for other functionality, notably heterocycles: as shown also in Scheme VI below, an ethylenediamine VI-4 was employed as a partner, in the presence of sulfur, imidazolines VI-5 were produced.

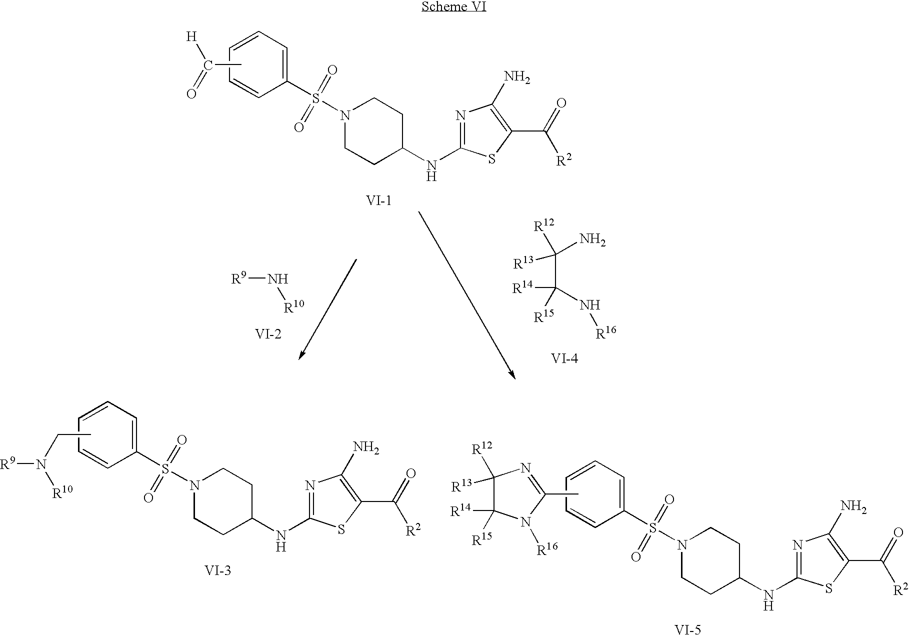

Similarly, other amines are available from aldehydes as shown below in Scheme VII. The aldehyde VII-1 underwent reductive amination similar to the protocol in Scheme VI to produce amines VII-2. The aldehyde VII-1 was available from careful acidic hydrolysis of the acetal VII-3, which in turn was produced upon alkylation of 2-chloropyridine V-1 (X=Cl, W=N, U, V=C) with glycolaldehyde dimethyl acetal. The sequence of Scheme VII was particularly useful to obtain these secondary amines VII-2, especially those not available from the straightforward protocol of Scheme V. 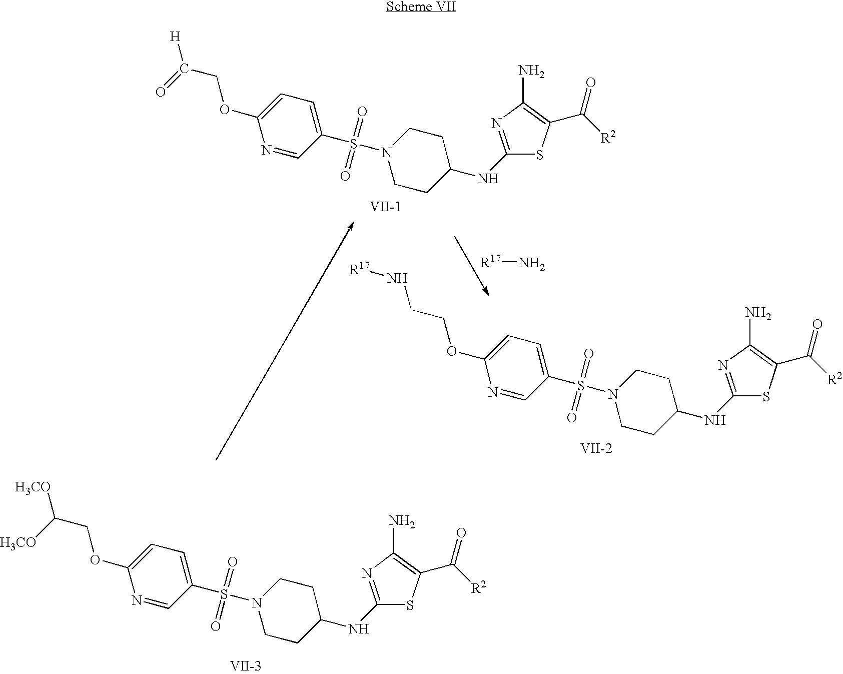
As shown in Scheme VIII, the nitrile VIII-1 was also a useful intermediate. Nitriles VIII-1 may be made according to the route in Scheme III from commercially available sulfonyl chloride III-7 (R₉=Ar—CN). Under routine conditions, the nitrile VIII-1 was converted to the amidine VIII-2. As well as good solubilizing groups, amidines are also potential starting materials for other heterocycles.

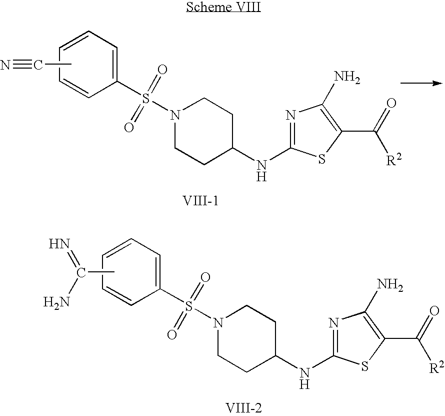

Another elaborative process adjacent to the arylsulfonamides is shown in Scheme IX below, to access thioalkyls in particular. The thiol IX-1 was easily available as the thiopyridine IX-1 (W=N) from the conversion of corresponding 2-chloropyridine V-1 (X=Cl, W=N, U, V=C) from Scheme V via substitution with sodium sulfide or an equivalent. Consequently the thiol IX-1 can be alkylated in straightforward manner to the thioalkyls IX-2.

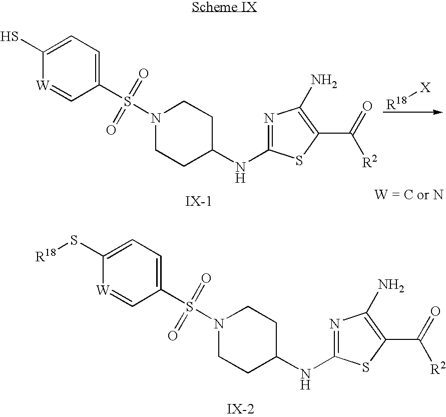

Another useful arylsulfonamide is shown below in Scheme X, the 2-vinyl heteroaryl X-1, formed through a Stille coupling of tributyl-vinyltin(IV) with 2-chloro-heteroaryl V-1 (X=Cl, W=N, U, V=C) from Scheme V. Amines, including anilines, can provide useful adducts X-2.

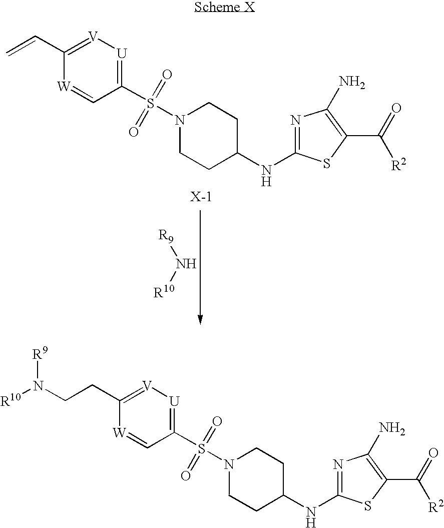

Another group of sulfonamides XI-3 and XI-4 result from further processing—subsequent to Scheme II—are shown in Scheme XI below. For example, commercially available 3-chloropropylsulfonyl chloride (III-7, R⁷=CH₂CH₂CH₂Cl) was used according to Scheme III with piperidine of type I-7 to selectively produce sulfonamide XI-1 where n=3. The terminal chloride of XI-1 (X=Cl) was typically converted in situ to the more reactive iodide XI-2 (X=I), which in turn alkylated secondary amines, or thiols to provide amino-alkylsulfonamides XI-3, or thio-alkyls XI-4, respectively. 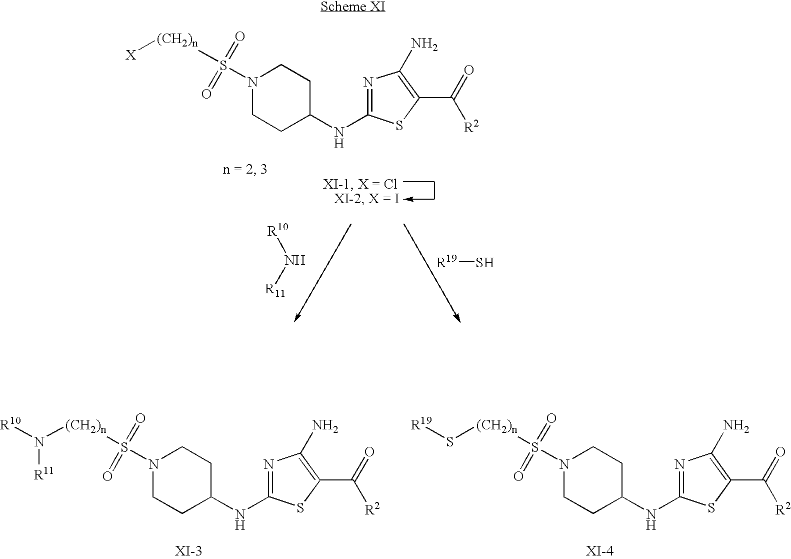
For sulfonamides like XI-3 and XI-4 with n=2 for the spacer group, as shown in Scheme XII, these were conveniently available via addition of amines or thiols to vinylsulfones XII-1. The production of adducts XII-2 or XII-3 was suitable for parallel, or combinatorial methods.

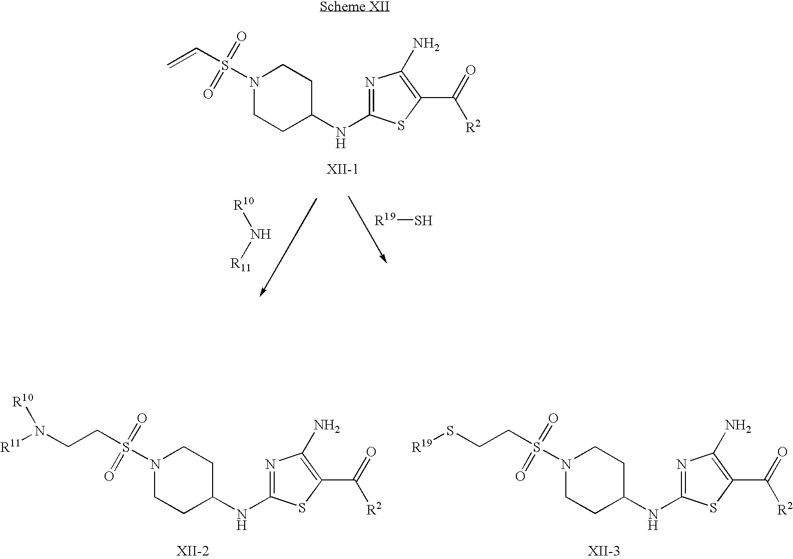

The following Examples will explain in more detail the method of preparing the representative compounds of the invention. In Examples, the structural formula indicates sometimes methyl group (—CH₃) as “—” for the simplicity. For Method diagram, the functional group such as R¹ or R² has the same meaning as defined above unless indicated otherwise.

### EXAMPLES

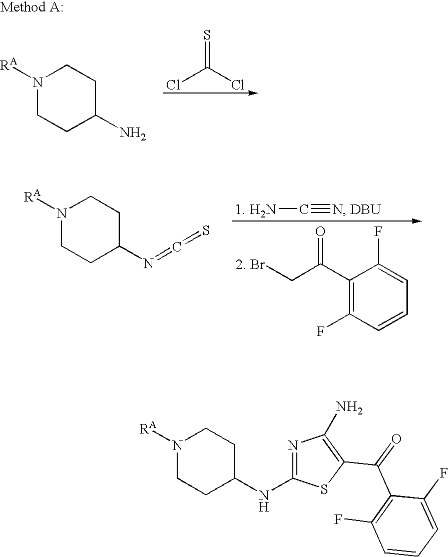

### Example A1

### 4-[4-Amino-5-(2,6-difluoro-benzoyl)-thiazol-2-ylamino]-piperidine-1-carboxylic Acid Ethyl Ester

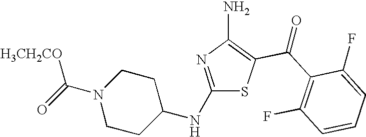

Starting materials were prepared as follows:
4-lsothiocyanato-piperidine-1-carboxylic Acid Ethyl Ester

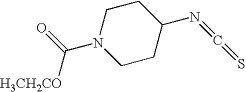

To a solution of 4-amino-piperidine-1-carboxylic acid ethyl ester (0.260 g, 1.50 mmol) and Et₃N (0.44 ml, 3.2 mmol) in CH₂Cl₂ at 0° C., thiophosgene (0.23 ml, 3.00 mmol) was added dropwise. The solution stirred at room temperature for 1 hour and diluted with CH₂Cl₂. The organic solution was then washed with sat. NaHCO₃, and brine, dried over MgSO₄, filtered, and concentrated to a syrup. Column chromatography (EtOAc/Hexane=2/1) afforded 0.20 g of solid in 40% yield, which was used without further purification.

¹H NMR (DMSO-d₆): δ 4.08-3.90 (m, 5H), 2.90 (m, 2H), 1.92 (m, 2H), 1.34 (m, 2H), 1.20 (t, 3H, J=7.1 Hz). IR (KBr): 2180 cm⁻¹.
2-Bromo-2′,6′-difluoroacetophenone

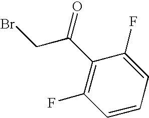

To a mechanically stirring solution of 2′,6′-difluoroacetophenone (100.0 g, 640.0 mmol; Melford Laboratories, Ltd.) in ethyl acetate (1300 ml) was added freshly milled copper(II) bromide (300 g, 1.35 mol) and bromine (1.6 ml, 32 mmol). The mixture was heated at reflux for 2.25 hours and allowed to cool to room temperature. The resultant green mixture was filtered and the solids rinsed with ethyl acetate (4×100 ml). The filtrate was concentrated with a rotary evaporator at <40° C. under reduced pressure, diluted with methyl t-butyl ether (MTBE; 650 ml), filtered through a pad of silica gel (230-400μ; 9.5 cm diam.×4 cm. ht.), and solids rinsed with MTBE (5×200 ml). Concentration of the filtrate gave a pale green oil, which was purified by fractional vacuum distillation to give 117 g of pale yellow oil, bp 88-97° C. (2.0 mm Hg) in 78% yield. Matched that previously described in World Patent Application WO99/21845 (in Example C (79)) and was used without any further purification or characterization.

¹H NMR: δ7.48 (ddd, 1H, J=6.3, 8.5, 14.8 Hz), 7.01 (ddd, 2H, J=4.6, 5.8, 16.6 Hz), 4.37 (t, 2H, J=0.7 Hz).

The title compound was prepared as follows. A solution of 4-isothiocyanate-piperidine-1-carboxylic acid ethyl ester (1.62 g, 7.60 mmol), DBU (1,8-diazabicyclo[5.4.0]undec-7-ene; 1.13 ml, 7.60 mmol), and cyanamide (0.45 g, 10.6 mmol) in acetonitrile stirred at room temperature for 45 minutes. 2-Bromo-2′,6′-difluoro-acetophenone (1.78 g, 7.60 mmol) and DBU (1.13 ml, 7.60 mmol) were added. After 2 hours, solvent was removed. A solution of the resultant residue in ethyl acetate was washed with sat. NaHCO₃, brine, dried over MgSO₄, filtered, and concentrated. Purification via column chromatography gave 2.20 g of solid in 66% yield.

¹H NMR (DMSO-d₆): δ 8.78 (br, 1H), 8.07 (br, 2H), 7.49 (m, 1H), 7.15 (t, 2H, J=8.8 Hz), 4.02 (q, 2H, J=7.1 Hz ), 3.82 (m, 3H), 2.85 (m, 2H), 1.82 (m, 2H), 1.31 (m, 2H), 1.18 (t, 3H, J=7.1 Hz). HRFABMS Calcd.for C₁₈H₂₁F₂N₄O₃S (MH⁺): 398.0051. Found: 398.0059. Anal. Calcd. For C₁₈H₂₀F₂N₄O₃S: C, 52.67; H, 4.91; N, 13.65; S, 7.81. Found: C, 52.72; H, 4.95; N, 13.64; S, 7.72.

### Example A2

### [4-Amino-2-(2,2,6,6-tetramethyl-piperidin-4-ylamino)-thiazol-5-yl]-(2,6-difluoro-phenyl)-methanone

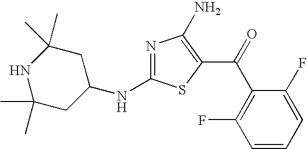

The title compound was prepared in a route with conditions similar to that for Example A1; originating from 2,2,6,6-tetramethyl-piperidin-4-ylamine.

¹H NMR (CDCl₃): δ 7.38 (m, 1H), 6.96 (m, 1H), 5.60 (br, 1H), 3.70 (br, 1H), 2.02 (m, 2H), 1.22 (s, 6H), 1.12 (s, 6H), 1.00 (m, 2H). HRMALDIMS. Calcd for C₁₉H₂₅F₂N₄OS (MH⁺): 395.1717. Found: 395.1725

### Example A3

### 1-[4-Amino-2-(1-benzyl-piperidin-4-ylamino)-thiazol-5-yl]-1-(2,6-difluoro-phenyl)-methanone

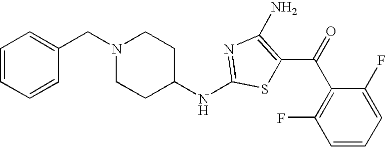

The title compound was prepared in a route with conditions similar to that for Example A1; originating from 4-amino-1-benzylpiperidine to give a brown solid in 43% yield after column chromatography.

¹H NMR (DMSO-d₆): δ 8.02 (bs, 2H), 7.50 (ddd, 1H, J=1.7, 6.7, 8.4 Hz), 7.38-7.22 (m, 5H), 7.12 (dd, 2H, J=7.6, 8.1 Hz), 3.48 (bs, 2H), 2.80-2.62 (m, 2H), 2.05-1.80 (m, 4H), 1.52-1.40 (m, 2H). HRMALDIMS. Calcd. for C₂₂H₂₃F₂N₄OS (MH⁺): 429.1555. Found: 429.1538. Anal. Calcd. for C₂₂H₂₂F₂N₄OS.0.6 H₂O: C, 60.15; H, 5.32; N, 12.75; S, 7.30. Found: C, 59.92; H, 5.09; N, 12.38; S, 7.13.

### Example A4

### 1-[4-Amino-2-(1-methyl-piperidin-4-ylamino)-thiazol-5-yl]-1-(2,6-difluoro-phenyl)-methanone

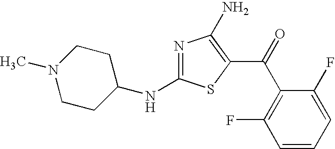

The title compound was prepared in a route with conditions similar to that for Example A1; originating from 1-methyl-piperidin-4-ylamine (Pau, et al Farmaco, 53, 233-240, (1998)) to give a yellow foam in 23% yield.

¹H NMR (DMSO-d₆): δ 8.08 (bs, 2H), 7.50 (ddd, 1H, J=1.7, 6.7, 8.4 Hz), 7.14 (dd, 2H, J=7.6, 15.8 Hz), 2.72 (bd, 2H, J=1.8 Hz), 2.14 (s, 3H), 2.00-1.82 (m, 3H), 1.52-1.42 (m, 2H). HRMALDIMS. Calcd. for C₁₆H₁₉F₂N₄OS (MH⁺): 353.1242. Found: 353.1258. Anal. Calcd. for C₁₆H₁₈F₂N₄OS.0.4 H₂O: C, 53.44; H, 5.27; N, 15.58; S, 8.92. Found: C, 53.30; H, 5.30; N, 15.20; S, 8.88.

### Example A5

### 4-[4-Amino-5-(2,6-difluoro-benzoyl)-thiazol-2-ylamino]-piperidine-1-carboxylic Acid tert-Butyl Ester

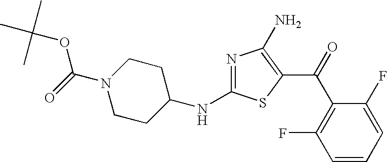

The title compound was prepared in a route similar to that for Example A1; originating from 4-amino-piperidine-1-carboxylic acid tert-butyl ester (initially purchased from AstaTech, Inc; but later prepared by following the method in U.S. Pat. No. 5,968,929).

¹H NMR: δ 7.39-7.28 (m, 1H), 6.94 (t, 2H, J=7.8 Hz), 5.54-5.49 (m, 1H), 4.11-4.00 (m, 2H), 3.58-3.43 (m, 2H), 2.94-2.82 (m, 2H), 2.08-1.98 (m, 2H), 1.45 (s, 9H).

### Example A6

### [4-Amino-2-(piperidin-4-ylamino)-thiazol-5-yl]-(2,6-difluoro-phenyl)-methanone

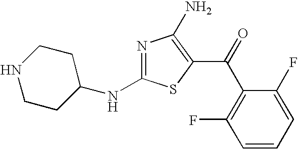

A solution of 4-[4-amino-5-(2,6-difluoro-benzoyl)-thiazol-2-ylamino]-piperidine-1-carboxylic acid tert-butyl ester (Example A5; 2.20 g, 5.02 mmol) in 30% TFA/CH₂Cl₂ (50 ml) stirred at room temperature for 90 minutes. The solvent was removed. A solution of the resultant residue in ethyl acetate was washed with sat. NaHCO₃, brine, dried over MgSO₄, filtered, and concentrated. The residue was triturated with ethyl ether and filtered to isolate 1.04 g of white solid in 61% yield.

¹H NMR (DMSO-d₆): δ 8.70 (bs, 1H), 8.08 (bs, 2H), 7.49 (ddd, 1H, J=6.6, 8.7, 15.0 Hz), 7.18 (ddd, 2H, J=1.8, 6.6, 15.6 Hz), 2.90 (d, 2H, J=12.3 Hz), 2.44 (t, 2H, J=11.4 Hz), 1.80 (d, 2H, J=11.4 Hz), 1.28 (ddd, 2H, J=4.2, 8.4, 11.4 Hz). HRMALDIMS. Calcd. for C₁₅H₁₆F₂N₄OS (MH⁺): 398.0051. Found: 398.0059. Anal. Calcd. for C₁₅H₁₆N₄OF₂S.1.5 H₂O: C, 49.31; H, 5.25; N, 15.33; S, 8.78. Found: C, 49.30; H, 5.04; N, 16.18; S, 8.63.

### Example A7

### 3-[4-Amino-5-(2,6-difluoro-benzoyl)-thiazol-2-ylamino]-piperidine-1-carboxylic Acid tert-Butyl Ester

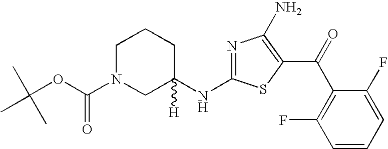

The title compound was prepared in a route with conditions similar to that for Example A1; originating from 3-amino-piperidine-1-carboxylic acid tert-butyl ester (de Costa, et al; J. Med. Chem. Vol. 35, pp. 4334-4343 (1992)) to give a brown foam in 100% crude yield, which was used without further purification.

¹H NMR (DMSO-d₆): δ 7.96 (2H, bs), 7.40 (1H, ddd, J=1.9, 6.7, 8.6 Hz), 7.06 (2H, t, J=8.1 Hz), 1.40 (9H, s).

### Example A8

### 1-[4-Amino-2-(piperidin-3-ylamino)-thiazol-5-yl]-1-(2,6-difluoro-phenyl)-methanone

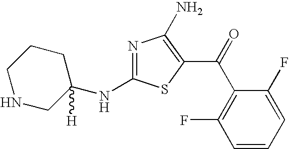

The title compound was prepared in a manner similar to that for Example A6 from 3-[4-amino-5-(2,6-difluoro-benzoyl)-thiazol-2-ylamino]-piperidine-1-carboxylic acid tert-butyl ester (Example A7) to give a brown foam in 80% crude yield, which was used without further purification.

¹H NMR (CD₃OD): δ 7.44 (ddd, 1H, J=2.0, 6.5, 8.5 Hz), 7.02 (dd, 2H, J=7.5, 8.3 Hz), 3.26-3.18 (m, 1H), 2.92 (dd, 1H, J=3.8, 13.1 Hz), 2.62-2.48 (m, 2H), 2.09-2.00 (m, 1H), 1.82-1.73 (m, 1H), 1.62-1.44 (m, 2H). LC-ESIMS (MH⁺): 339

### Example A9

### 3RS-[4-Amino-5-(2,6-difluoro-benzoyl)-thiazol-2-ylamino]-pyrrolidine-1-carboxylic acid tert-butyl ester

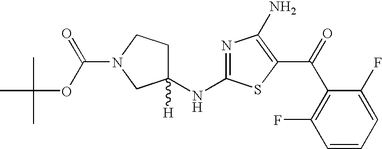

The starting materials were prepared as follows:
3RS-Amino-pyrrolidine-1-carboxylic acid tert-butyl ester

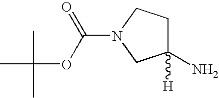

To a solution of 3-aminopyrrolidine (0.86 g, 10 mmol) in CHCl₃ (50 ml) at 0° C. was added dropwise a solution of di-t-butyl dicarbonate ((Boc)₂O; 2.06 g, 10 mmol) in CHCl₃ (50 ml). The mixture stirred at room temperature for 1 hour, and then washed with brine, dried over K₂CO₃, filtered, and concentrated to give 1.8 g of yellow oil in 98% yield, which was used without further purification.

¹H NMR: δ 3.60-3.28 (m, 4H), 3.02 (m, 1H), 2.04 (m,1H), 1.64 (m, 1H), 1.45 (s, 9H), 1.45-1.20 (m, 2H).

The title compound was prepared in a route with conditions similar to that for Example A1; originating from 3-amino-pyrrolidine-1-carboxylic acid tert-butyl ester.

¹H NMR (DMSO-d₆): δ 8.05 (br, 2H), 7.50 (m, 1H), 7.17 (dd, 2H, J=7.6, 8.4 Hz), 1.40 (s, 9H).

### Example A10

### 1-[4-Amino-2-(pyrrolidin-3RS-ylamino)-thiazol-5-yl]-1-(2,6-difluoro-phenyl)-methanone

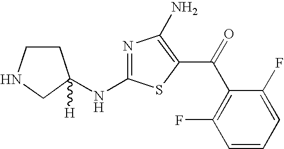

The title compound was prepared in a manner similar to that for Example A6 from 3RS-[4-amino-5-(2,6-difluoro-benzoyl)-thiazol-2-ylamino]-pyrrolidine-1-carboxylic acid tert-butyl ester.

¹H NMR (DMSO-d₆): δ 8.05 (br, 2H), 7.50 (m, 1H), 7.17 (dd, 2H, J=7.6, 8.4 Hz). LC-ESIMS (MH⁺): 325

### Example A11

### 1-[4-Amino-2-(pyrrolidin-3S-ylamino)-thiazol-5-yl]-1-(2,6-difluoro-phenyl)-methanone

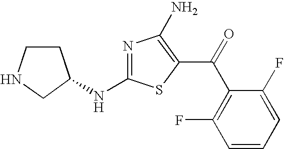

The starting material 3S-amino-pyrrolidine-1-carboxylic acid tert-butyl ester was prepared in a manner similar to that for 3RS-amino-pyrrolidine-1-carboxylic acid tert-butyl ester in Example A9 from 3S-amino-pyrrolidine.

The intermediate 3S-[4-amino-5-(2,6-difluoro-benzoyl)-thiazol-2-ylamino]-pyrrolidine-1-carboxylic acid tert-butyl ester was prepared in a manner similar to that for preparation of Example A9 from 3S-amino-pyrrolidine-1-carboxylic acid tert-butyl ester.

The title compound was prepared in a manner similar to that for preparation of Example A6 from 3S-[4-amino-5-(2,6-difluoro-benzoyl)-thiazol-2-ylamino]-pyrrolidine-1-carboxylic acid tert-butyl ester.

The spectra data were identical to that of Example A10.

### Example A12

### 3-[4-Amino-5-(2,6-difluoro-benzoyl)-thiazol-2-ylamino]-azetidine-1-carboxylic acid tert-butyl ester

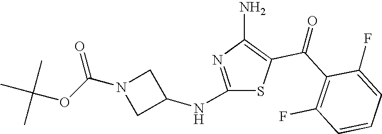

The starting materials were prepared as follows:
3-Methanesulfonyloxy-azetidine-1-carboxylic acid tert-butyl ester

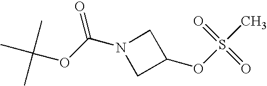

To a solution of 3-methanesulfonatoazetidinium chloride (1.05 g, 5.65 mmol; Anderson, et al., J. Org. Chem., Vol. 37, pp. 3953-3955 (1972)) in CH₂Cl₂ (30 ml) was added Et₃N (1.57 ml, 11.3 mmol) and (t-BOC)₂O (1.23 g, 5.65 mmol). After 3 h, the mixture was washed with sat. NH₄Cl (25 ml) and H₂O (25 ml), dried over MgSO₄, filtered, and concentrated in vacuo to afford a yellow oil, which was purified via column chromatography with 50% EtOAc/hexanes as eluant to give 0.55 g of yellow oil in 38% yield, which was used without any further purification.

¹H NMR: δ 5.12-4.88 (1H, m), 3.02 (3H, s), 1.25 (9H, s).
3-Azido-azetidine-1-carboxylic acid tert-butyl ester

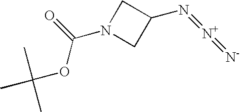

To a solution of 3-methanesulfonyloxy-azetidine-1-carboxylic acid tert-butyl ester (540 mg, 2.15 mmol) in DMF (3 ml) was added NaN₃ (0.279 g, 4.29 mmol). The mixture was heated at 85° C. After 48 hours, the mixture was allowed to cool and diluted with diethyl ether (50 ml). The organic layer was washed with H₂O (2×250 ml) and brine (25 ml), dried over MgSO₄, filtered, and concentrated in vacuo to afford 425 mg of a yellow oil in 100% yield, which was used without further purification.

¹H NMR: δ 1.52 (9H, s).
3-Amino-azetidine-1-carboxylic acid tert-butyl ester

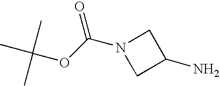

To a solution of 3-azido-azetidine-1-carboxylic acid tert-butyl ester (0.420 g, 2.19 mmol) in EtOAc (20 ml) was added 10% Pd-C (100 mg). The resultant suspension stirred under an atmosphere of H₂ (balloon). After 12 hours, the mixture was filtered through a pad of Celite. The filtrate was concentrated in vacuo to give 1.76 g of a colorless oil in 99% yield, which was used without further purification.

¹H NMR: δ 1.50 (9H, s).
3-Isothiocyanato-azetidine-1-carboxylic acid tert-butyl ester

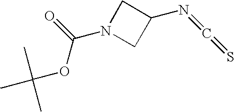

This compound was prepared in a manner analogous to that for 4-isothiocyanato-piperidine-1-carboxylic acid ethyl ester for Example A1. 3-Amino-azetidine-1-carboxylic acid tert-butyl ester provided a brown oil in 99% yield, which was used without further purification.

¹H NMR: d 1.50 (9H, s).

The title compound was prepared in a manner analogous to that for Example A1. 3-Isothiocyanato-azetidine-1-carboxylic acid tert-butyl ester and 2-bromo-2′,6′-difluoro-acetophenone provided a brown foam in 77% yield, which was typically used without further purification.

¹H NMR: δ 7.33-7.15 (1H, m), 6.88-6.78 (2H, m), 1.32 (9H, s).

### Example A13

### 1-[4-amino-2-(azetidin-3-ylamino)-thiazol-5-yl]-1-(2,6-difluoro-benzoyl)-methanone

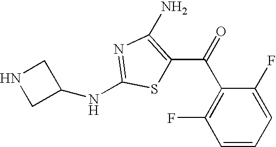

The title compound was prepared in a manner similar to that for Example A6, from 3-[4-amino-5-(2,6-difluoro-benzoyl)-thiazol-2-ylamino]-azetidine-1-carboxylic acid tert-butyl ester (Example A12), and used without further purification.

¹H NMR (DMSO-d₆): δ 8.08 (bs, 2H), 7.50 (ddd, 1H J=1.5, 8.2, 15.0 Hz), 7.15 (dd, 2H, J=7.7, 8.0 Hz) LC-ESIMS (MH⁺): 311

### Example A14

### [4-Amino-2-(1-benzhydryl-azetidin-3-ylamino)-thiazol-5-yl]-(2,6-difluoro-phenyl)-methanone

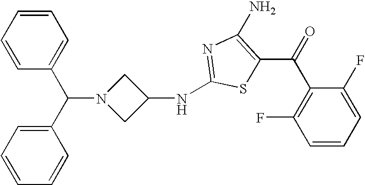

The starting material was prepared as follows:
3-Azido-1-(1,1-diphenyl-methyl)-azetidine

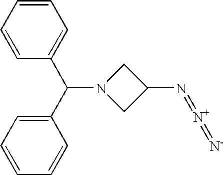

The starting material was prepared in a manner similar to that for 3-azido-azetidine-1l-carboxylic acid tert-butyl ester in Example A12 from 1-benzylhydryl-3-methanesulfonatoazetidine (Anderson, et. al., J. Org. Chem., Vol. 37, pp. 3953-3955, (1972)), to provide a yellow foam in 88% yield and used without further purification.

¹HNMR (CD₃OD): δ 7.42-7.13 (10H, m), 4.40 (1H, s), 4.10-4.02 (1H, m), 3.50-3.42 (2H, m), 3.06-2.98 (2H, m).
1-(1,1-Diphenyl-methyl)-azetidin-3-ylamine

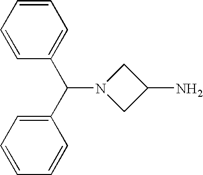

This compound was prepared in a manner similar to that for 3-amino-azetidine-1-carboxylic acid tert-butyl ester in Example A12 from 3-azido-1-(1,1-diphenyl-methyl)-azetidine in 40% yield, which was used without further purification.

¹H NMR: δ 4.08 (s, 1H), 3.44-3.36 (m, 1H), 3.32 (ddd, 2H, J=1.6, 6.3, 8.6 Hz), 2.43 (ddd, 2H, J=1.6, 6.3, 8.6 Hz)

The title compound of this Example was prepared in a route similar to that for Example A1, originating from 1-(1,1-diphenyl-methyl)-azetidin-3-ylamine.

¹H NMR (DMSO-d₆): δ 8.02 (bs, 2H), 7.56-7.10 (m, 13H), 4.42 (s, 1H), 3.42 (dd, 2H, J=7.3, 7.4 Hz), 2.92 (dd, 2H, J=6.6, 7.1 Hz). HRMALDIMS. Calcd. for C₂₆H₂₃F₂N₄OS (MH⁺): 477.1555. Found: 477.1566. Anal. Calcd. for C₂₆H₂₂F₂N₄OS.0.2 CHCl₃.0.15 CH₃CN: C, 62.83; H, 4.51; N, 11.47; S, 6.33. Found: C, 62.66; H, 4.56; N, 11.82; S, 6.32.

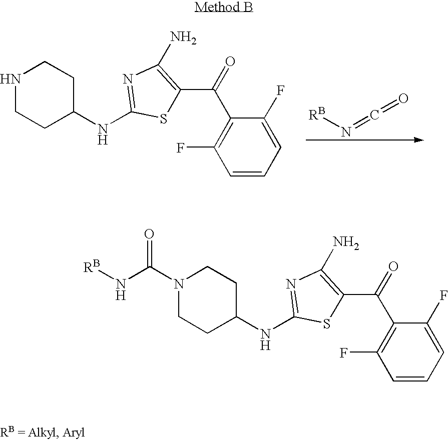

### Example B1

### 4-[4-Amino-5-(2,6-difluoro-benzoyl)-thiazol-2-ylamino]-piperidine-1-carboxylic Acid Isopropylamide

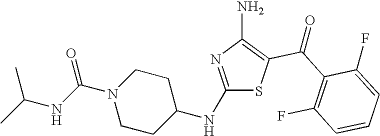

The title compound was prepared as follows:

A solution of [4-amino-2-(piperidin-4-ylamino)-thiazol-5-yl]-(2,6-difluoro-phenyl)-methanone (Example A6; 52 mg, 0.15 mmol) and isopropyl isocyanate (39 mg, 0.46 mmol) in DMF (6 ml) was stirred at room temperature overnight. Solvent was removed under reduced pressure. A solution of the resultant residue in ethyl acetate was washed with sat. NaHCO₃, dried with MgSO₄, filtered, and concentrated. Reversed phase preparative HPLC afforded 54 mg of solid in 85% yield.

¹H NMR (DMSO-d₆): δ 8.72 (br, 1H), 8.09 (s, 2H), 7.54-7.41 (m, 1H), 7.22-7.10 (m, 2H, 2H), 6.15 (s, 1H, 1H), 3.92-3.81 (m, 3H), 3.79-3.62 (m, 1H), 2.82-2.64 (m, 2H), 1.89-1.73 (m, 2H), 1.38-1.22 (m, 2H), 1.04 (s, 3H), 1.02 (s, 3H). HRMALDIMS. Calcd for C₁₉H₂₃F₂N₅O₂SNa (M+Na⁺): 446.1438. Found: 446.1455

### Example B2

### 4-[4-Amino-5-(2,6-difluoro-benzoyl)-thiazol-2-ylamino]-piperidine-1-carboxylic Acid (4-Dimethylamino-phenyl)-amide

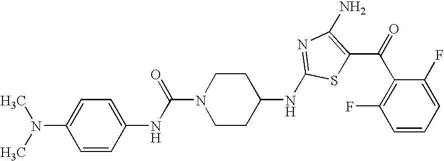

The title compound was prepared in a manner similar to that for Example B1 from [4-amino-2-(piperidin-4-ylamino)-thiazol-5-yl]-(2,6-difluoro-phenyl)-methanone (Example A6) and 4-dimethylamino-phenyl isothiocyanate (Lancaster).

¹H NMR (DMSO-d₆): δ 7.57-7.40 (m, 1H), 7.23-7.07 (m, 5H), 6.63 (d, 2H, J=9.2 Hz,), 4.14-3.90 (m, 3H), 2.98-2.82 (m, 2H), 2.74 (s, 3H), 1.97-1.78 (m, 2H), 1.48-1.24 (m, 2H). HRMALDIMS. Calcd for C₂₄H₂₆F₂N₆O₂SNa (M+Na⁺): 523.1704. Found: 523.1724

### Example B3

### 4-[4-Amino-5-(2,6-difluoro-benzoyl)-thiazol-2-ylamino]-piperidine-1-carboxylic Acid (1R-Phenyl-ethyl)-amide

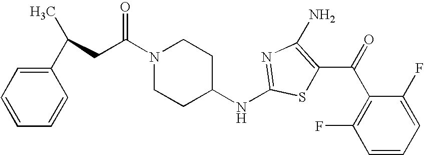

The title compound was prepared in a manner similar to that used to prepare the compound of Example B1 from [4-amino-2-(piperidin-4-ylamino)-thiazol-5-yl]-(2,6-difluoro-phenyl)-methanone (Example A6) and R-(+)-α-methylbenzyl isocyanate.

¹H NMR (DMSO-d₆): δ 7.52-7.40 (m, 1H), 7.34-7.21 (m, 4H), 7.19-7.08 (m, 3H), 6.77-6.67 (m, 1H), 4.87-4.72 (m, 1H), 3.98-3.83 (m, 3H), 2.96-2.68 (m, 2H), 1.92-1.77 (m, 2H), 1.32-1.12 (m, 2H). HRMALDIMS. Calcd for C₂₄H₂₅F₂N₅O₂SNa (M+Na⁺): 508.1595. Found: 508.1600

### Example B4

### 4-[4-Amino-5-(2,6-difluoro-benzoyl)-thiazol-2-ylamino]-piperidine-1-carboxylic Acid (2,5-Dimethoxy-phenyl)-amide

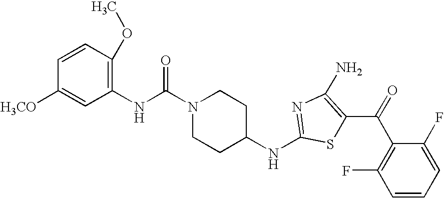

The title compound was prepared in a manner similar to that used to prepare the compound of Example B1 from [4-amino-2-(piperidin-4-ylamino)-thiazol-5-yl]-(2,6-difluoro-phenyl)-methanone (Example A6) and 2,5-dimethoxyphenyl isocyanate (Carbolabs).

¹H NMR (DMSO-d₆): δ 7.55-7.42 (m, 1H), 7.34 (d, 1H, J=3.2 Hz), 7.20-7.09 (m, 2H), 6.89 (d, 1H, J=8.9 Hz), 6.57-6.50 (dd, 1H, J=3.2, 8.9 Hz), 3.98-3.74 (m, 3H), 3.53 (s, 6H), 3.07-2.76 (m, 2H), 1.96-1.65 (m, 2H), 1.49-1.30 (m, 2H). HRMALDIMS. Calcd for C₂₄H₂₅F₂N₅O₄S (MH⁺): 518.1674. Found: 518.1653

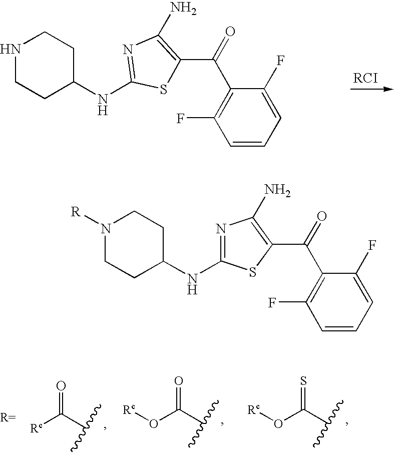

### Example C1

### {4-Amino-2-[1-(4-iodo-benzoyl)-piperidin-4-ylamino]-thiazol-5-yl}-(2,6-difluoro-phenyl)-methanone

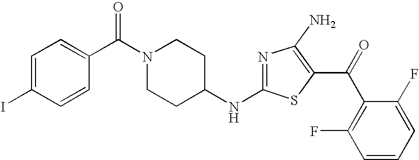

To a solution of 1-[4-amino-2-(piperidin-4-ylamino)-thiazol-5-yl]-1-(2,6-difluoro-phenyl)-methanone (Example A6; 200 mg, 0.59 mmol) in a mixture of THF (3 ml) and acetonitrile (3 ml) was added diisopropylamine (0.20 ml, 1.2 mmol) and 4-iodo-benzoyl chloride (173 mg, 0.649 mmol). After 1 hour, the reaction mixture was diluted with ethyl acetate (50 ml) and the resultant organic solution was washed with sat. NH₄Cl (25 ml) and H₂O (25 ml), dried over MgSO₄, filtered, and concentrated to afford a brown foam, which was purified via preparative TLC (2 mm) with 10% MeOH/CHCl₃ as eluant to give 266 mg of yellow solid in 78% yield.

¹H NMR (DMSO-d₆): δ 7.82 (s, 2H), 7.60 (d, 2H, J=8.0 Hz), 7.22-7.22 (m, 1H), 7.00-6.90 (m, 4H), 3.55-3.40 (m, 1H), 3.12-2.90 (m, 2H), 1.98-1.82 (m, 2H), 1.48-1.30 (m, 2H), 1.08-0.90 (m, 2H). HRMALDIMS. Calcd. for C₂₂H₂₀F₂IN₄O₂S (MH⁺): 579.0314. Found: 579.0309. Anal. Calcd. for C₂₂H₁₉F₂IN₄O₂S: C, 44.24; H, 3.30; N, 9.17; S, 5.25. Found: C, 44.14; H, 3.67; N, 8.85; S, 4.87.

### Example C2

### {4-Amino-2-[1-(4-methoxy-benzoyl)-piperidin-4-ylamino]-thiazol-5-yl}-(2,6-difluoro-phenyl)-methanone

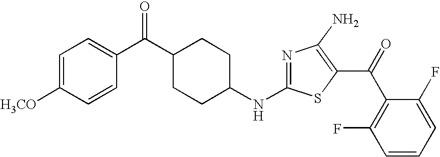

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 4-methoxy-benzoyl chloride.

¹H NMR (DMSO-d₆): δ 7.94 (s, 2H), 7.42-7.34 (m, 1H), 7.22 (d, 2H, J=8.7 Hz), 7.05 (dd, 2H, J=7.7, 8.2 Hz), 6.88 (d, 2H, J=8.8 Hz), 3.78 (s, 3H), 3.10-3.00 (m, 2H), 1.98-1.82 (m, 2H), 1.42-1.32 (m, 2H). HRMALDIMS. Calcd. for C₂₃H₂₃F₂N₄O₃S (MH⁺): 473.1453. Found: 473.1432. Anal. Calcd. for C₂₃H₂₂F₂N₄O₃S.0.3 CHCl₃: C, 55.05; H, 4.42; N, 11.02; S, 6.31. Found: C, 54.82; H, 4.48; N, 10.99; S, 6.33.

### Example C3

### 4-[4-Amino-5-(2,6-difluoro-benzoyl)-thiazol-2-ylamino]-piperidine-1-carboxylic Acid 4-Chloro-phenyl Ester

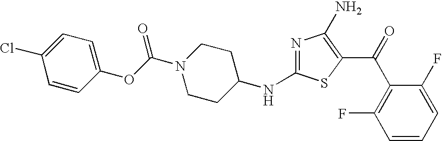

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 4-chloro-benzoyl chloride.

¹H NMR (DMSO-d₆): δ 8.02 (s, 1H), 7.52-7.38 (m, 4H), 7.25-7.13 (m, 3H), 4.15-3.87 (m, 2H), 1.98-1.72 (m, 2H), 1.55-1.37 (m, 2H), 1.27-1.17 (m, 2H). HRMALDIMS. Calcd. for C₂₂H₂₀ClF₂N₄O₃ (MH⁺): 493.0907. Found: 493.0900. Anal. Calcd. for C₂₂H₁₉ClF₂N₄O₃S.0.3 CHCl₃.0.7 H₂O: C, 49.926; H, 3.89; Cl, 11.59; N, 10.46; S, 5.99. Found: C, 50.15; H, 3.86; Cl, 11.50; N, 10.23; S, 6.01.

### Example C4

### 4-{4-[4-Amino-5-(2,6-difluoro-benzoyl)-thiazol-2-ylamino]-piperidine-1-carbonyl}-benzoic Acid Methyl Ester

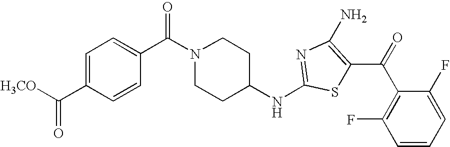

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 4-chlorocarbonyl-benzoic acid methyl ester (TCI) to give a yellow solid in 61% yield.

¹H NMR (DMSO-d₆): δ 8.05-7.97 (m, 4H), 7.55-7.38 (m, 3H), 7.15 (t, 2H, J=7.9 Hz), 3.88 (s, 3H), 3.57-3.40 (m, 1H), 3.30-2.95 (m, 2H), 2.05-1.85 (m, 2H), 1.57-1.37 (m, 2H). HRMALDIMS. Calcd. for C₂₄H₂₃F₂N₄O₄S (MH⁺): 501.1403. Found: 501.1410. Anal. Calcd. for C₂₄H₂₂F₂N₄O₄S.0.5 H₂O: C, 56.57; H, 4.77; N, 11.00; S, 6.29. Found: C, 56.65; H, 4.58; N, 10.76; S, 6.16.

### Example C5

### (4-Amino-2-{1-[3-chloro-4-(propane-2-sulfonyl)-thiophene-2-carbonyl]-piperidin-4-ylamino}-thiazol-5-yl)-(2,6-difluoro-phenyl)-methanone

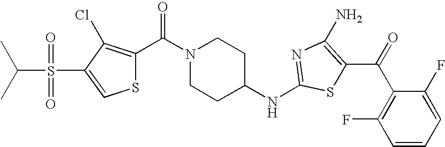

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 3-chloro-4-(isopropyl-sulfonyl)-thiophene-2-carbonyl chloride (Maybridge) to give a yellow powder in 84% yield.

¹H NMR (DMSO-d₆): δ 8.60 (s, 1H), 7.55-7.42 (m, 1H), 7.18 (t, 2H, J=7.5 Hz), 3.53-3.42 (d, 1H, J=6.8 Hz), 2.02-1.92 (m, 2H), 1.52-1.42 (m, 2H), 1.28 (s, 3H), 1.22 (s, 3H), 0.95 (bd, 2H, J=5.4 Hz). HRMALDIMS. Calcd. for C₂₃H₂₄ClF₂N₄O₄S₃ (MH⁺): 589.0611. Found: 589.0618. Anal. Calcd. for C₂₃H₂₃ClF₂N₄O₄S₃.0.1 Hexane.0.5 Et₂O.0.45 CHCl₃: C, 45.44; H, 4.37; 8.14; S, 13.97; Cl, 12.10. Found: C, 45.62; H, 4.25; N, 8.50; S, 13.67; Cl, 11.97.

### Example C6

### 4-[4-Amino-5-(2,6-difluoro-benzoyl)-thiazol-2-ylamino]-piperidine-1-carbothioic Acid O-Phenyl Ester

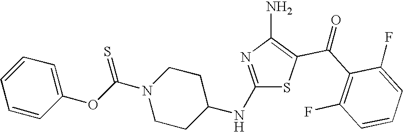

The title compound was prepared in a manner similar to that for Example C1 from [4-amino-2-(piperidin-4-ylamino)-thiazol-5-yl]-(2,6-difluoro-phenyl)-methanone (Example A6) and phenyl chlorothionoformate to furnish a brown foam in 86% yield.

¹H NMR (DMSO-d₆): δ 8.08 (bs, 2H), 7.58-7.44 (m, 1H), 7.38 (t, 2H, J=7.6 Hz), 7.26-7.12 (m, 3H), 7.05 (d, 2H, J=7.5 Hz), 4.70 (d, 1H, J=13.8 Hz), 4.48 (d, 1H, J=13.8 Hz), 3.58-3.35 (m, 2H), 2.02 (d, 2H, J=9.3 Hz), 1.60-1.48 (m, 2H). HRMALDIMS. Calcd. for C₂₂H₂₁F₂N₄O₂S₂ (MH⁺): 475.1068. Found: 475.1075. Anal. Calcd. for C₂₂H₂₀F₂N₄O₂S₂.0.4 CHCl₃: C, 51.51; H, 3.94; N, 10.73; S 12.28. Found: C, 51.75; H, 4.03; N, 10.58; S, 12.06.

### Example C7

### 1-{4-[4-Amino-5-(2,6-difluoro-benzoyl)-thiazol-2-ylamino]-piperidin-1-yl}-3-(2-chloro-3,4-dimethoxy-phenyl)-propenone

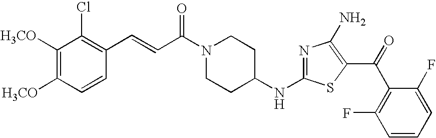

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and (E)-3-(2-chloro-3,4-dimethoxy-phenyl)-acryloyl chloride (Maybridge) to provide a yellow solid in 46% yield.

¹H NMR (DMSO-d₆): δ 8.05 (bs, 2H), 7.78 (d, 1H, J=3.1 Hz) 7.74 (d, 1H, J=9.6 Hz), 7.58-7.45 (m, 1H), 7.22-7.08 (m, 4H), 4.38-4.15 (m, 2H), 3.90 (s, 3H), 3.74 (s, 3H), 3.00-2.80 (m, 1H), 1.98 (d, 2H, J=10.6 Hz), 1.48-1.30 (m, 2H). HRMALDIMS. Calcd. for C₂₆H₂₆ClF₂N₄O₄S (MH⁺): 563.1326. Found: 563.1336. Anal. Calcd. for C₂₆H₂₅ClF₂N₄O₄S.0.35 CHCl₃: C, 52.33; H, 4.22; N, 9.26; S, 5.30. Found: C, 52.46; H, 4.21; N, 9.33; S, 5.38.

### Example C8

### {4-Amino-2-[1-(3-chloro-thiophene-2-carbonyl)-piperidin-4-ylamino]-thiazol-5-yl}-(2,6-difluoro-phenyl)-methanone

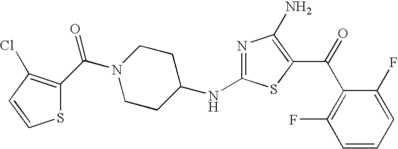

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 3-chloro-thiophene-2-carbonyl chloride to give a yellow foam in 77% yield.

¹H NMR (DMSO-d₆): δ 8.08 (bs, 2H), 7.80 (d, 1H, J=5.2 Hz), 7.52-7.42 (m, 1H), 7.18 (t, 2H, J=7.7 Hz), 7.12 (d, 1H, J=5.2 Hz). 3.20-3.05 (m, 2H), 1.98 (d, 2H, J=9.5 Hz), 1.50-1.38 (m, 2H). HRMALDIMS. Calcd. for C₂₀H₁₈ClF₂N₄O₂S₂ (MH⁺): 483.0528. Found: 483.0536. Anal. Calcd. for C₂₀H₁₇ClF₂N₄O₂S₂.0.1 Hexane.0.35 CHCl₃: C, 47.18; H, 3.54; Cl, 13.63; N, 10.50; S, 12.02. Found: C, 47.06; H, 3.45; Cl, 13.96; N, 10.34; S, 11.70.

### Example C9

### 1-(4-Amino-2-{1-[-(6-chloro-pyridin-3-yl)-methanoyl]-piperidin-4-ylamino}-thiazol-5-yl)-1-(2,6-difluoro-phenyl)-methanone

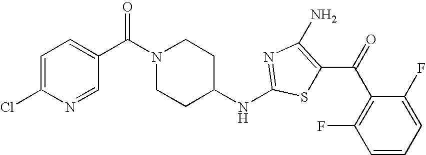

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 6-chloro-nicotinoyl chloride to give a yellow powder in 45% yield.

¹H NMR (DMSO-d₆): δ 8.38 (dd, 1H, J=2.4, 0.6 Hz), 7.79 (dd, 1H, J=2.4, 8.2 Hz), 7.47 (dd, 1H, J=0.6, 8.2 Hz), 7.37 (m, 1H), 6.95 (dd, 2H, J=7.4, 8.2 Hz), 4.43 (m, 1H), 3.88 (m, 1H), 3.61 (m, 1H), 2.12-1.92 (m, 2H), 1.60-1.38 (m, 2H). HRFABMS Calcd. For C₂₁H₁₈F₂N₅O₂SClNa (M+Na⁺): 500.0730. Found: 500.0735. Anal. Calcd. for C₂₁H₁₈F₂N₅O₂SCl.0.3 CH₂Cl₂.0.2 MeOH: C, 50.65; H, 3.84; N, 13.74; S, 6.29. Found: C, 50.42; H, 3.84; N, 13.74; S, 6.34.

### Example C10

### 1-{4-Amino-2-[1-(1-isoxazol-5-yl-methanoyl)-piperidin-4-ylamino]-thiazol-5-yl}1-(2,6-difluoro-phenyl)-methanone

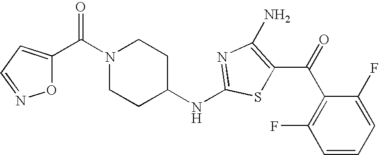

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and isoxazole-5-carbonyl chloride to give a yellow powder in 65% yield.

¹H NMR (DMSO-d₆): δ 8.89 (br, 1H), 8.79 (d, 1H, J=1.9 Hz), 8.11 (br, 2H), 7.55 (m, 1H), 7.22 (dd, 2H, J=7.7, 8.1 Hz), 6.97 (d, 1H, J=1.9 Hz), 4.33 (m, 1H), 3.82 (m, 1H), 3.13 (m, 1H), 2.14-1.97 (m, 2H), 1.60-1.44 (m, 2H). HRFABMS Calcd. For C₁₉H₁₈F₂N₅O₃S (MH⁺): 434.1093. Found: 434.1113. Anal. Calcd. for C₁₉H₁₇F₂N₅O₃S.0.3 CH₂Cl₂.0.1 hexane: C, 51.12; H, 4.10; N, 14.98; S, 6.86. Found: C, 51.20; H, 4.18; N, 14.75; S, 6.80.

### Example C11

### 4-[4-Amino-5-(2,6-difluoro-benzoyl)-thiazol-2-ylamino}-piperidine-1-carbothioic acid —O-(4-Fluoro-phenyl) ester

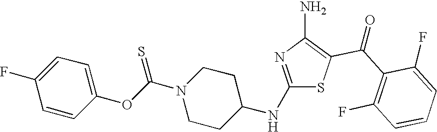

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 4-fluoro-phenyl chlorothionoformate to give a yellow solid in 100% yield.

¹H NMR (DMSO-d₆): δ 8.78 (br, 1H), 7.99 (br, 2H), 7.42 (m, 1H), 7.17-6.98 (m, 6H), 4.59 (m, 1H), 4.40 (m, 1H), 3.55-3.28 (m, 2H), 2.20-1.91 (m, 2H), 1.55-1.39 (m, 2H). HRFABMS. Calcd. For C₂₂H₂₀F₃N₄O₂S₂ (MH⁺): 493.0974. Found: 493.0977. Anal. Calcd. for C₂₂H₁₉F₃N₄O₂S₂.0.3 CH₂Cl₂.0.3 hexane: C, 53.22; H, 4.41; N, 10.30; S, 11.79. Found: C, 53,58; H, 4.37; N, 10.11; S, 11.64.

### Example C12

### 1-(4-Amino-2-{1-[1-(3-nitro-phenyl)-methanoyl]-piperidin-4-ylamino}-thiazol-5-yl)-1-(2,6-difluoro-phenyl)-methanone

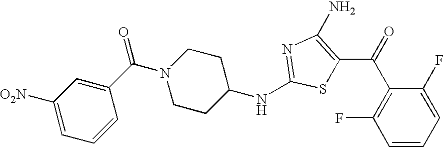

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 3-nitro-benzoyl chloride to give a yellow solid in 100% yield.

¹H NMR (DMSO-d₆): δ 8.90 (br, 1H), 8.41 (dd, 1H, J=1.2, 8.1 Hz), 8.28 (t, 1H, J=1.6 Hz), 8.17 (br, 2H), 7.95 (dt, 1H, J=1.2, 6.4 Hz), 7.87 (d, 1H, J=8.1 Hz), 7.60 (m, 1H), 7.27 (dd, 2H, J=7.6, 8.1 Hz), 4.40 (m, 1H), 3.55-3.28 (m, 2H), 3.2 (m, 1H), 2.20-1.91 (m, 2H), 1.70-1.48 (m, 2H). HRFABMS. Calcd. For C₂₂H₁₉F₂N₅O₄SNa (M+Na⁺): 510.1018. Found: 510.1023. Anal. Calcd. for C₂₂H₁₉F₂N₅O₄S.0.5 CH₂Cl₂.0.3 hexane: C, 52.51; H, 4.39; N, 12.60; S, 5.77. Found: C, 52.55; H, 4.33; N, 12.49; S, 5.83.

### Example C13

### {4-[4-Amino-5-(2,5-difluoro-benzoyl)-thiazol-2-ylamino]-piperidin-1-yl}-pyridin-4-yl-methanone

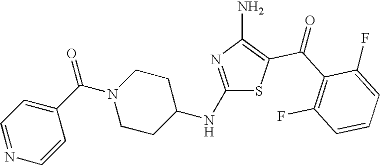

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and isonicotinoyl chloride.

¹H NMR (DMSO-d₆): δ 8.84 (br, 1H), 8.68 (d, 2H, J=5.9 Hz), 8.08 (bs, 2H), 7.56-7.42 (m, 1H), 7.37 (d, 2H, J=5.9 Hz), 7.18 (m, 2H), 4.38 (m, 1H), 3.49 (m, 1H), 3.19-3.01 (m, 3H), 2.06 (m, 2H), 1.57 (m, 2H). HRMALDIMS. Calcd. For C₂₁H₂₀F₂N₅O₂SNa (M+Na⁺): 543.0278. Found: 543.0271.

### Example C14

### 1-{4-Amino-2-[1-(1-1H-imidazol-4-yl-methanoyl)-piperidin-4-ylamino]-thiazol-5-yl}-1-(2,6-difluoro-phenyl)-methanone

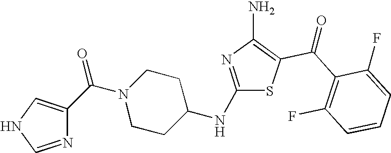 1H-lmidazole-4-carbonyl Chloride Hydrochloride

As suggested by Moss, et al J. Amer. Chem. Soc., 109, 6209-6210 (1987), a suspension of 1H-imidazole-4-carboxylic acid (575 mg, 5.13 mmol) in thionyl chloride (25 ml) was heated at reflux for 3 days. The solution was allowed to cool to ambient temperature and concentrated in vacuo to afford 800 mg of yellow powder in 94% yield, which was used without further purification.

¹H NMR (DMSO-d₆): δ 8.86 (s, 1H), 8.22 (s, 1H).

The title compound was prepared in a manner similar to that for Example C1 from [4-amino-2-(piperidin-4-ylamino)-thiazol-5-yl]-(2,6-difluoro-phenyl)-methanone (Example A6) and 1H-imidazole-4-carbonyl chloride hydrochloride to give a yellow foam in 26% yield.

¹H NMR (DMSO-d₆): δ 8.08 (bs, 2H), 7.70 (s, 1H), 7.58 (s, 1H), 7.48 (ddd, 1H, J=1.9, 6.7, 8.2 Hz), 7.94 (dd, 2H, J=7.7, 8.1 Hz), 1.98-1.74 (m, 2H), 1.48-1.30 (m, 2H). HRMALDIMS. Calcd. for C₁₉H₁₉F₂N₆O₂S (MH⁺): 433.1253. Found: 433.1268. Anal. Calcd. for C₁₉H₁₈F₂N₆O₂S.81.0 H₂O: C, 50.66; H, 4.48; N, 18.66; S, 7.12. Found: C, 50.70; H, 4.52; N, 18.53; S, 6.94.

### Example C15

### 1-(4-Amino-2-{1-[1-(3-methyl-3H-imidazol-4-yl)-methanoyl]-piperidin-4-ylamino}-thiazol-5-y)-1-(2,6-difluoro-phenyl)-methanone

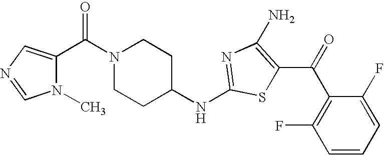

3-Methyl-3H-imidazole-4-carbonyl chloride hydrochloride was prepared in manner similar to that for 1H-imidazole-4-carbonyl chloride hydrochloride in Example C14 from 3-methyl-3H-imidazole-4-carboxylic acid (O'Connell, et al, Synthesis, pp. 767-771 (1998)) to give a yellow solid in 46% yield.

¹H NMR (DMSO-d₆): δ 9.29 (s, 1H), 8.29 (d, 1H, J=1.5 Hz).

The title compound was prepared in a manner similar to that used to prepare the compound of Example C1 from [4-amino-2-(piperidin-4-ylamino)-thiazol-5-yl]-(2,6-difluoro-phenyl)-methanone (Example A6) and 3-methyl-3H-imidazole-4-carbonyl chloride hydrochloride.

¹H NMR (DMSO-d₆): δ 8.08 (bs, 2H), 7.72 (s, 1H), 7.50 (ddd, 1H, J=1.5, 6.8, 8.2 Hz), 7.22-7.12 (m, 3H), 4.22-4.08 (m, 2H), 3.68 (s, 3H), 3.20-3.05 (m, 2H), 2.02-1.92 (bd, 2H, J=12.0 Hz), 1.50-1.36 (m, 2H). HRMALDIMS. Calcd. for C₂₀H₂₁F₂N₆O₂S (MH⁺): 447.1409. Found: 447.1421. Anal. Calcd. for C₂₀H₂₀F₂N₆O₂S.1.0 H₂O: C, 51.72; H, 4.77; N, 18.09; S, 6.90. Found: C, 51.47; H, 4.84; N, 17.65; S, 6.93.

### Example C16

### 4-[4-Amino-5-(2,6-difluoro-benzoyl)-thiazol-2-ylamino]-piperidine-1-carboxylic acid 4-nitro-phenyl ester

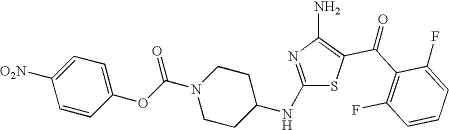

The title compound was prepared in a manner similar to that for Example C1 from [4-amino-2-(piperidine-4-ylamino)-thiazol-5-yl]-(2,6-difluoro-phenyl)-methanone (Example A6; 0.10 g, 030 mmol) and bis-(4-nitrophenyl) carbonate in DMF, without base. Reversed phase preparative HPLC provided 45 mg of yellow powder in 32% yield.

¹H NMR (DMSO-d₆): δ 8.82 (br, 1H), 8.29 (m, 2H), 8.09 (br, 2H), 7.40-7.58 (m, 3H), 7.18 (t, 2H, J=8.7 Hz), 4.02 (m, 2H), 3.03-3.21 m, 3H), 2.03 (m, 2H), 1.51 (m, 2H). FABMS (MH⁺): 504. Anal. Calcd. for C₂₂H₁₉F₂N₅O₅S.0.3 EtOAc: C, 52.59; H, 4.09; N, 13.17; S, 6.03. Found: C, 52.88; H, 4.18; N, 13.17; S, 6.02.

### Example C17

### {4-[4-Amino-5-(2,6-difluoro-benzoyl)-thiazol-2-ylamino]-piperidin-1-yl}-imidazol-1-yl-methanone

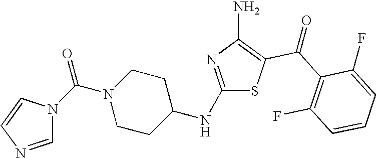

The title compound was prepared in a manner similar to that used to prepare the compound of Example C16 from [4-amino-2-(piperidin-4-ylamino)-thiazol-5-yl]-(2,6-difluoro-phenyl)-methanone (Example A6) and 1,1′-carbonyldiimidazole.

¹H NMR (DMSO-d₆): δ 8.89 (bs, 1H), 8.10 (bs, 2H), 8.02 (s, 1H), 7.57 (m, 1H), 7.42 (s, 1H), 7.18 (m, 1H), 7.02 (s, 1H), 3.90-3.78 (m, 3H), 3.29 (m, 2H), 2.08 (m, 2H), 1.62 (m, 2H). LC-ESIMS (MH⁺): 433 Anal. Calcd. For C₁₉H₁₈F₂N₆O₂S.0.15 H₂O.0.18 EtOAc: C, 52.51; H, 4.41; N, 18.63; S, 7.11. Found: C, 52.67; H, 4.50; N, 18.93; S, 6.97.

### Example C18

### {4-Amino-2-[1-(4-bromo-benzoyl)-pyrrolidin-3-ylamino]-thiazol-5-yl}-(2,6-difluoro-phenyl)-methanone

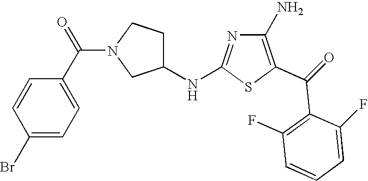

The title compound was prepared in a manner similar to that used in preparation of the compound of Example C1 from 1-[4-amino-2-(pyrrolidin-3-ylamino)-thiazol-5-yl]-1-(2,6-difluoro-phenyl)-methanone (Example A10) and 4-bromo-benzoyl chloride to give a yellow powder in 82% yield.

¹H NMR (DMSO-d₆): δ 9.01 (br, 1H), 8.05 (d, 2H, J=13.5 Hz), 7.65 (dd, 2H, J=4.0, 8.1 Hz), 7.48 (br, 1H), 7.47 (d, 2H, J=7.8 Hz), 7.19 (d, 1H, J=7.8 Hz), 7.14 (d, 1H, J=7.8 Hz), 4.24 (m, 1H), 3.75 (m, 1H), 3.64-3.40 (m, 3H), 2.15 (m, 1H), 1.95 (m, 1H). Anal. Calcd. for C₂₂H₁₇BrF₂N₄O₂S.0.1 CH₃OH: C, 49.34; H, 3.66; N, 10.70; S, 6.13. Found: C, 49.54; H, 3.38; N, 11.04; S, 6.00.

### Example C19

### {4-Amino-2-[1-(3-nitro-benzoyl)-azetidin-3-ylamino]-thiazol-5-yl}-(2,6-difluoro-phenyl)-methanone

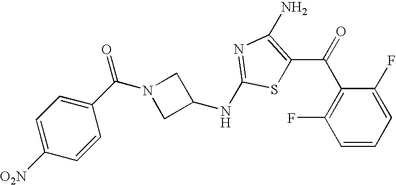

The title compound was prepared in a manner similar to that used in preparation of the compound of Example C1 from 1-[4-amino-2-(azetidin-3-ylamino)-thiazol-5-yl]-1-(2,6-difluoro-phenyl)-methanone (Example A13) and 4-nitro-benzoyl chloride to give a yellow solid in 13% yield.

¹H NMR (DMSO-d₆): δ 8.42-8.34 (m, 2H), 8.08 (s, 2H), 8.02 (s, 1H), 7.82-7.74 (m, 1H), 7.58-7.44 (m, 1H), 7.18 (dd, 2H, J=7.7, 8.1 Hz). HRMALDIMS. Calcd. for C₂₀H₁₆N₅O₄S (MH⁺): 460.0886. Found: 460.0896. Anal. Calcd. for C₂₀H₁₅N₅O₄S.0.5 EtOAc.0.05 CHCl₃: C, 52.16; H, 3.79; N, 13.79; S, 6.32.

Found: C, 52.18; H, 3.85; N, 13.96; S, 5.96.

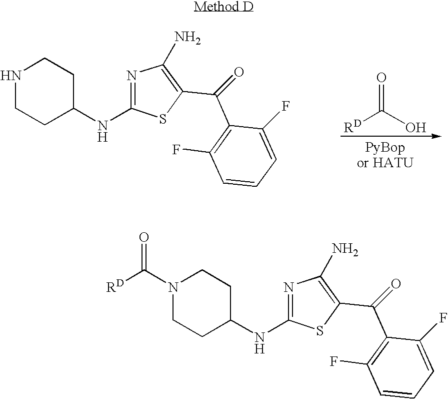

### Example D1

### 1-(4-Amino-2-{1-[1-(1-methyl-piperidin-4-yl)-methanoyl]-piperidin-4-ylamino}-thiazol-5-yl)-1-(2,6-difluoro-phenyl)-methanone

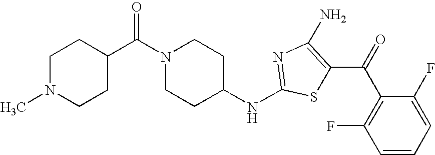

A solution of [4-amino-2-(piperidin-4-ylamino)-thiazol-5-yl]-(2,6-difluoro-phenyl)-methanone (Example A6; 300 mg, 1.0 mmol), 1-methyl-piperidine-4-carboxylic acid (230 mg, 1.25 mmol), benzotriazol-1-yl-oxy-tris-pyrrolidino-phosphonium hexafluorophosphate (PyBop; 572 mg, 1.1 mmol), and triethylamine (604 mg, 6.0 mmol) in DMF (10 ml) stirred at room temperature for 60 minutes. The solvent was removed under reduced pressure. A solution of the resultant residue in ethyl acetate was washed with sat. NaHCO₃, dried over MgSO₄, filtered, and concentrated. Purification via reversed phase preparative HPLC provided yellow solid in 65% yield.

¹H NMR (DMSO-d₆): δ 8.81 (br, 1H), 8.08 (s, 2H), 7.61-7.42 (m, 1H), 7.27-7.08 (m, 2H), 4.31-4.13 (m, 2H), 3.98-3.79 (m, 3H), 3.39-3.11 (m, 3H), 2.92-2.64 (m, 4H), 2.28 (s, 3H), 2.12-1.77 (m, 4H), 1.41-1.14 (m, 2H). HRMALDIMS. Calcd for C₂₂H₂₇F₂N₅O₂SNa (M+Na^{+):} 486.1751. Found: 486.1757

The following compounds of Examples D2 through D13 were prepared in a manner similar to that for Example D1 above from [4-amino-2-(piperidin-4-ylamino)-thiazol-5-yl]-(2,6-difluoro-phenyl)-methanone (Example A6) and corresponding commercially available carboxylic acids.

### Example D2

### (4-[4-Amino-5-(2,6-difluoro-benzoyl)-thiazol-2-ylamino}-piperidin-1-yl)-2-dimethylamino-ethanone

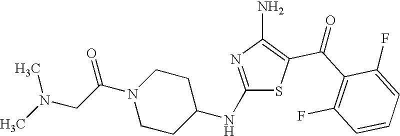

¹H NMR (DMSO-d₆): δ 8.77 (br, 1H), 8.08 (s, 2H), 7.59-7.43 (m, 1H), 7.27-7.14 (m, 2H), 4.31-4.19 (m, 2H), 3.99-3.83 (m, 2H), 3.20-3.02 (m, 1H), 2.84-2.69 (m, 2H), 2.50 (s, 6H), 1.98-1.84 (m, 2H), 1.53-1.24 (m, 2H). HRMALDIMS. Calcd. for C₁₉H₂₄F₂N₅O₂S (MH⁺): 424.1619. Found: 424.1610

### Example D3

### 1-(4-[4-Amino-5-(2,6-difluoro-benzoyl)-thiazol-2-ylamino}-piperidin-1-yl)-3-piperidin-1-yl-propan-1-one

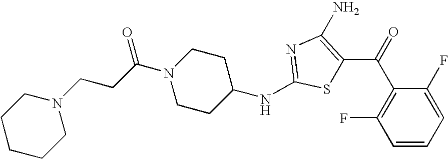

¹H NMR (DMSO-d₆): δ 8.77 (br, 1H), 8.06 (s, 2H), 7.59-7.44 (m, 1H), 7.22-7.10 (m, 2H), 4.27-4.13 (m, 2H), 3.88-3.76 (m, 2H), 3.50-3.38 (m, 1H), 3.21-3.07 (m, 2H), 2.86-2.63 (m, 2H), 2.03-1.84 (m, 2H), 1.67-1.18 (m, 7H). HRMALDIMS. Calcd. for C₂₃H₂₉F₂N₅O₂SNa (M+Na⁺): 500.1908. Found: 500.1912

### Example D4

### (4-[4-Amino-5-(2,6-difluoro-benzoyl)-thiazol-2-ylamino}-piperidin-1-yl)-2S-dimethylamino-phenyl-propan-1-one

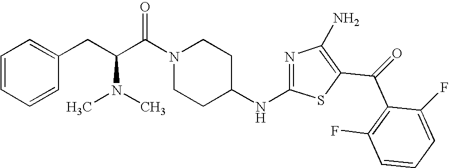

¹H NMR (DMSO-d₆): δ 8.75 (br, 1H), 8.03 (s, 2H), 7.56-7.48 (m, 1H), 7.27-7.02 (m, 8H), 4.28-4.13 (m, 2H), 3.93-3.70 (m, 3H), 3.12-2.91 (m, 1H), 2.90-2.52 (m, 2H), 2.32 (s, 6H), 1.88-1.59 (m, 2H), 1.41-1.08 (m, 2H). HRMALDIMS. Calcd. for C₂₆H₃₀F₂N₅O₂S (MH⁺): 514.2088. Found: 514.2102

### Example D5

### 5S-[1{-4-[4-Amino-5-(2,6-difluoro-benzoyl)-thiazol-2-ylamethano]-piperidin-1-yl}-methanoyl]-tetrahydro-furan-2-one

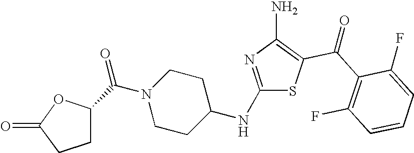

¹H NMR (DMSO-d₆): δ 8.82 (br, 1H), 8.11 (s, 2H), 7.62-7.46 (m, 1H), 7.29-7.13 (m, 2H), 5.61-5.48 (m, 1H), 4.31-4.13 (m, 2H), 3.92-3.77 (m, 2H), 3.37-3.13 (m, 2H), 3.01-2.74 (m, 2H), 2.28-2.12 (m, 1H), 2.07-1.90 (m, 2H), 1.59-1.28 (m, 2H). ESIMS (MH⁺): 451, (M−H⁻): 449. Anal. Calcd. for C₂₀H₂₀F₂N₄O₄S: C, 53.33; H, 4.48; N, 12.44; S, 7.12. Found: C, 53.34; H, 4.60; N, 2.29; S, 6.93.

### Example D6

### 1-{[4-Amino-5-(2,6-difluoro-benzoyl)-thiazol-2-ylamino]-piperidin-1-yl)3-pyridin-4-yl-prop-2(E)-enone

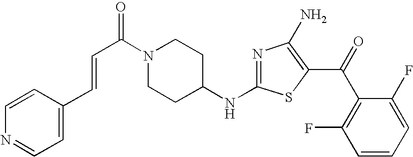

¹H NMR (DMSO-d₆): δ 8.8 (br, 1H), 8.64-8.57 (m, 2H), 8.07 (s, 2H), 7.73-7.64 (m, 2H), 7.58-7.37 (m, 1H), 7.22-7.12 (m, 2H), 4.39-4.15 (m, 2H), 3.34-3.19 (m, 3H), 2.04-1.88 (m, 2H), 1.50-1.28 (m, 2H). HRMALDIMS. Calcd. for C₂₃H₂₂F₂N₅O₂S (MH⁺): 470.1957. Found: 470.1474

### Example D7

### 1-(4-Amino-2-{1-[1-(4-chloro-3-methyl-phenyl)-methanoyl]-piperidin-4-ylamino}-thiazol-5-yl)-1-(2,6-difluoro-phenyl)-methanone

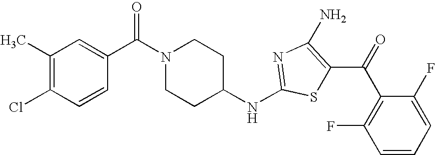

¹H NMR (DMSO-d₆): δ 8.80 (br, 1H), 8.12 (s, 2H), 8.62-8.43 (m, 2H), 8.38 (s, 1H), 8.30-8.14 (m, 3H), 4.40-4.16 (m, 1H), 3.69-3.43 (m, 2H), 3.22-2.93 (m, 2H), 2.30 (s, 3H), 2.03-1.80 (m, 2H), 1.52-1.31 (m, 2H). ESIMS (MH⁺): 491. Anal. Calcd. for C₂₃H₂₁ClF₂N₄O₂S.0.1 Et₂O: C, 56.39; H, 4.45; N, 11.24; S, 6.43. Found: C, 56.15; H, 4.64; N, 0.97; S, 6.23.

### Example D8

### 1-(4-Amino-2-{1-[1-(3-chloro-4-fluoro-phenyl)-methanoyl]-piperidin-4-ylamino}-thiazol-5-yl)-1-(2,6-difluoro-phenyl)-methanone

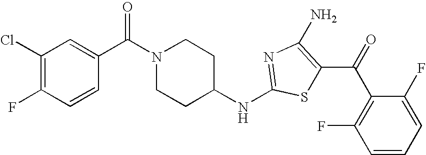

¹H NMR (DMSO-d₆): δ 8.72 (br, 1H), 8.01 (s, 2H), 8.61-8.52 (m, 1H), 8.50-8.30 (m, 3H), 8.18-8.04 (m, 2H), 4.32-4.10 (m, 1H), 3.60-3.37 (m, 2H), 3.17-2.88 (m, 2H), 2.01-1.79 (m, 2H), 1.51-1.28 (m, 2H). ESIMS (MH⁺): 495. Anal. Calcd. for C₂₂H18ClF₃N₄O₂S.0.25 EtOAc: C, 53.44; H, 3.90; N, 10.84; S, 6.20. Found: C, 53.17; H, 3.88; N, 10.61; S, 6.06.

### Example D9

### 1-{4-[4-Amino-5-(2,6-difluoro-benzoyl)-thiazol-2-ylamino]-piperidin-1-yl}-4-p-tolyl-but-2(E)-ene-1,4-dione

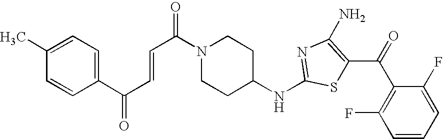

¹H NMR (DMSO-d₆): δ 8.80 (br, 1H), 8.06 (s, 2H), 7.86 (d, 2H, J=8.3 Hz), 7.68 (d, 1H, J=15.3 Hz,), 7.56-7.35 (m, 4H), 7.22-7.12 (m, 2H), 4.36-4.22 (m, 1H), 4.05-3.87 (m, 2H), 3.04-2.86 (m, 2H), 2.39 (s, 3H), 2.01-1.89 (m, 2H), 1.55-1.29 (m, 2H). ESIMS (MH⁺): 511. Anal. Calcd. for C₂₆H₂₄F₂N₄O₃S.0.15 EtOAc: C, 60.99; H, 4.85; N, 10.70; S, 6.12. Found: C, 60.75; H, 4.91; N, 10.63; S, 6.00.

### Example D10

### 1-{4-[4-Amino-5-(2,6-difluoro-benzoyl)-thiazol-2-ylamino]piperidin-1-yl}-2-(3,5-dimethyl-phenyl)-ethanone

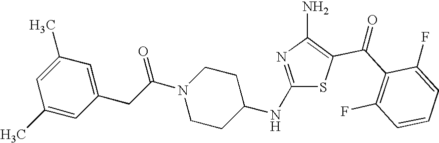

¹H NMR (DMSO-d₆): δ 8.70 (br, 1H), 8.03 (s, 2H), 7.56-7.40 (m, 1H), 7.22-7.08 (m, 2H), 7.89-7.78 (m, 3H), 4.32-4.17 (m, 1H), 3.93-3.78 (m, 1H), 3.60 (s, 2H), 3.17-3.00 (m, 2H), 2.82-2.63 (m, 1H), 2.20 (s, 6H), 1.94-1.81 (m, 2H), 1.39-1.17 (m, 2H). ESIMS (MH⁺): 485. Anal. Calcd. for C₂₅H₂₆F₂N₄O₂S: C, 61.97; H, 5.41; N, 11.56; S, 6.62. Found: C, 61.71; H, 5.51; N, 11.48; S, 6.49.

### Example D11

### {4-[4-Amino-5-(2,5-difluoro-benzoyl)-thiazol-2-ylamino]-piperidin-1-yl}-(4-bromo-phenyl)-methanone

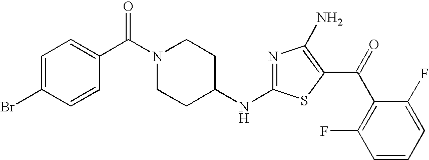

¹H NMR (DMSO-d₆): δ 8.81 (br, 1H), 8.09 (bs, 2H), 7.67 (d, 2H, J=8.2 Hz), 7.58-7.42 (m, 1H), 7.36 (d, 2H, J=8.2 Hz), 7.18 (m, 2H), 4.30 (m, 1H), 3.61 (m, 1H), 2.90-3.19 (m, 3H), 1.98 (m, 2H), 1.52 (m, 2H). HRMALDIMS. Calcd. for C₂₂H₂₀F₂N₄O₂SNa (MNa⁺): 543.0278. Found: 543.0271.

### Example D12

### 1-[4-Amino-2-{1-[-(3-methoxy-4-methyl-phenyl)-methanoyl]-piperidin-4-ylamino}-thiazol-5-yl)-1-(2,6-difluoro-phenyl)-methanone Trifluoroacetic Acid Salt

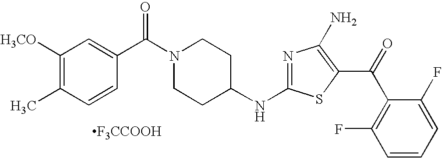

Purified via preparative HPLC.

¹H NMR (CD₃OD): δ 7.56-7.42 (m, 1H), 7.21 (d, 2H, J=7.4 Hz), 7.08 (m, 2H), 6.90-6.84 (m, 2H), 4.50 (br, 1H), 4.08-3.83 (m, 2H; s, 3H), 3.22 (m, 2H), 2.21 (s, 3H), 2.17 (m, 2H), 1.68 (m, 2H). HRMALDIMS. Calcd. For C₂₄H₂₅F₂N₄O₃S (MH⁺): 487.1610. Found: 487.1621. Anal. Calcd. for C₂₄H₂₄F₂N₄O₃S.0.90 TFA: C, 52.59; H, 4.26; N, 9.51; S, 5.44. Found: C, 52.59; H, 4.34; N, 9.70; S, 5.44.

### Example D13

### 12(Z)-(1-{4-[4-Amino-5-(2,6-difluoro-benzoyl)-thiazol-2-ylamino]-piperidin-1-yl}-methanoyl)-3-(3-hydroxy-phenyl)-acrylonitrile Trifluoroacetic Acid Salt

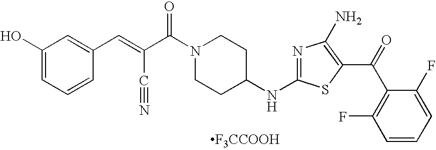

Purified via preparative HPLC.

¹H NMR (CD₃OD): δ 7.51 (s, 1H), 7.41-7.20 (m, 4H), 7.98-7.83 (m, 3H), 4.24-3.91 (m, 3H), 3.19 (m, 2H), 2.09 (m, 2H), 1.59 (m, 2H). HRMALDIMS. Calcd. For C₂₄H₂₂F₂N₄O₃S (MH⁺): 532.1225. Found: 532.1215. Anal. Calcd. For C₂₄H₂₁F₂N₄O₃S.1.25 TFA: C, 50.65; H, 3.44; N, 10.74; S, 4.92. Found: C, 50.66; H, 3.54; N, 10.84; S, 4.91.

### Example D14

### {4-Amino-2-[1-(3,5-dimethyl-benzoyl)-piperidin-4-ylamino]-thiazol-5-yl}-(2,6-difluoro-phenyl)-methanone

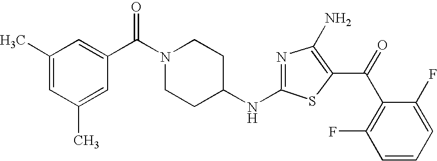

To a solution of 1-[4-amino-2-(piperidin-4-ylamino)-thiazol-5-yl]-1-(2,6-difluoro-phenyl)-methanone (Example A6; 150 mg, 0.44 mmol) in DMF (3 ml) was added 3,5-dimethyl-benzoic acid (132 mg, 0.88 mmol), O-(7-azabenzotriazol-1-yl)-1,1,3,3-tetramethyluronium hexafluorophosphate (HATU; 200 mg, 0.53 mmol] and triethylamine (184 μl, 1.32 mmol). After 3 hours, the mixture was diluted with ethyl acetate (50 ml). The organic solution was washed with H₂O (2×25 ml), sat. NaHCO₃ (2×25 ml), and brine (25 ml), dried over Na₂SO₄, filtered, and concentrated in vacuo to afford a brown foam, which was purified via preparative TLC (2 mm) to provide a yellow foam in 53% yield.

¹H NMR (DMSO-d₆): δ 8.08 (bs, 2H), 7.52-7.42 (m, 1H), 7.18 (t, 2H, J=7.8 Hz), 7.06 (s, 1H), 6.92 (s, 2H), 3.12-2.92 (m, 2H), 2.28 (s, 6H), 2.00-1.82 (m, 2H), 1.48-1.30 (m, 2H). HRMALDIMS. Calcd. for C₂₄H₂₅F₂N₄O₂S (MH⁺): 471.1661. Found: 471.1681. Anal. Calcd. for C₂₄H₂₄F₂N₄O₂S.0.3 H₂O: C, 60.57; H, 5.21; N, 11.77; S, 6.74. Found: C, 60.32; H, 5.13; N, 11.89; S, 6.62.

The following compounds of Examples D15 to D19 were prepared in a manner similar to that used to prepare the compound of Example D14 above from 1-[4-amino-2-(piperidin-4-ylamino)-thiazol-5-yl]-1-(2,6-difluoro-phenyl)-methanone (Example A6) and corresponding carboxylic acids, using HATU as a coupling reagent.

### Example D15

### {4-Amino-2-[1-(3,4-dimethyl-benzoyl)-piperidin-4-ylamino]-thiazol-5-yl}-(2,6-difluoro-phenyl)-methanone

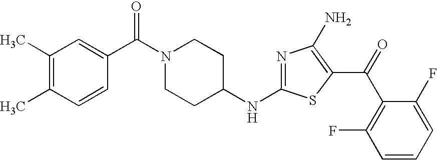

¹H NMR (DMSO-d₆): δ 8.08 (bs, 2H), 7.55-7.42 (m, 1H), 7.24-7.12 (m, 3H), 7.08 (d, 1H, J=7.6 Hz), 3.18-2.92 (m, 2H), 2.22 (s, 6H), 2.00-1.82 (m, 2H), 1.50-1.32 (m, 2H). HRMALDIMS. Calcd. for C₂₄H₂₅F₂N₄O₂S (MH⁺): 471.1661. Found: 471.1684. Anal. Calcd. for C₂₄H₂₄F₂N₄O₂S.0.4 H₂O: C, 60.34; H, 5.23; N, 11.73; S, 6.71. Found: C, 60.15; H, 5.20; N, 11.90; S, 6.65.

### Example D16

### 1-{4-[4-Amino-5-(2,6-difluoro-benzoyl)-thiazol-2-ylamino]-piperidin-1-yl}-pent-2(E)-ene-1,4-dione

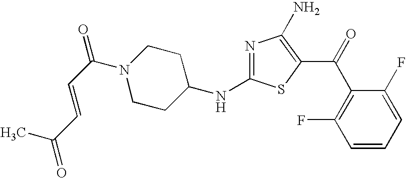

¹H NMR (DMSO-d₆): δ 8.08 (bs, 2H), 7.52-7.42 (m, 1H), 7.40 (d, 1H, J=15.8 Hz), 7.16 (t, 2H, J=8.0 Hz), 6.62 (d, 1H, J=15.8 Hz), 4.24 (bd, 1H, J=13.6 Hz), 4.05-3.95 (m, 1H), 2.90 (dd, 1H, J=11.2, 12.9 Hz), 2.32 (s, 3H), 2.00-1.84 (m, 2H), 1.50-1.30 (m, 2H) HRMALDIMS. Calcd. for C₂₀H₂₁F₂N₄O₃S (MH⁺): 435.1297. Found: 435.1303. Anal. Calcd. for C₂₀H₂₀F₂N₄O₃S.0.2 H₂O: C, 54.61; H, 4.72; N, 12.74; S, 7.29. Found: C, 54.35; H, 4.68; N, 12.66; S, 7.08.

### Example D17

### {4-Amino-2-[1-(3,5-dimethoxy-4-methyl-benzoyl)-piperidin-4-ylamino]-thiazol-5-yl-}(2,6-difluoro-phenyl)-methanone

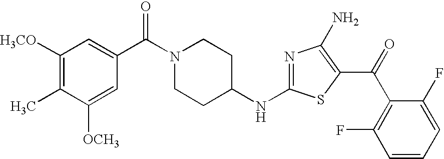

¹H NMR (DMSO-d₆): δ 8.08 (bs, 2H), 7.56-7.44 (m, 1H), 7.18 (dd, 2H, J=7.7, 8.1 Hz), 6.60 (s, 2H), 3.80 (s, 6H) 3.20-3.00 (m, 2H), 2.02 (s, 3H), 2.00-1.88 (m, 2H), 1.50-1.38 (m, 2H). HRMALDIMS. Calcd. for C₂₅H₂₇F₂N₄O₄S (MH⁺): 517.1716. Found: 517.1691. Anal. Calcd. for C₂₅H₂₆F₂N₄O₄S.0.4 H₂O: C, 57.33; H, 5.16; N, 10.70; S, 6.12. Found: C, 57.14; H, 5.11; N, 10.76; S, 6.00.

### Example D18

### 1-{4-[4-Amino-5-(2,6-difluoro-benzoyl)-thiazol-2-ylamino]-piperidin-1-yl}3(Z)-(2-methoxy-phenyl)-propenone

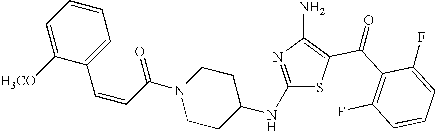

¹H NMR: δ 8.02 (bs, 2H), 7.52-7.42 (m, 1H), 7.30-7.20 (m, 2H), 7.15 (dd, 2H, J=7.8, 8.1 Hz), 7.02 (d, 1H, J=7.8 Hz), 6.80 (dd, 1H, J=7.0, 7.6 Hz), 6.78 (d, 1H, J=12.6 Hz), 6.10 (d, 1H, J=12.6 Hz), 4.20 (d, 1H, J=13.3 Hz), 3.80 (s, 3H), 3.68 (d, 1H, J=13.6 Hz), 3.00-2.78 (m, 2H), 1.92-1.80 (m, 1H), 1.70-1.62 (m, 1H), 1.32-1.20 (m, 1H), 0.95-0.82 (m, 1H). HRMALDIMS. Calcd. for C₂₅H₂₄F₂N₄O₃SNa (MNa⁺): 521.1429. Found: 521.1431. Anal. Calcd. for C₂₅H₂₄F₂N₄O₃S.0.4 H₂O: C, 59.37; H, 4.94; N, 11.08; S, 6.34. Found: C, 59.27; H, 4.93, N, 11.12; S, 6.31.

### Example D19

### {4-Amino-2-[1-(5-chloro-2-methoxy-benzoyl)-piperidin-4-ylamino]-thiazol-5-yl}-(2,6-difluoro-phenyl)-methanone

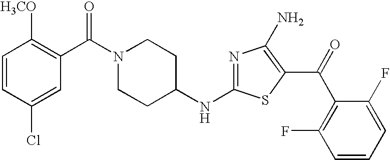

¹H NMR (DMSO-d₆): δ 8.08 (bs, 2H), 7.52-7.40 (m, 2H), 7.22-7.10 (m, 4H), 4.32 (bd, 2H, J=12.6 Hz), 3.80 (s, 3H), 3.12-2.90 (m, 2H), 2.02-1.92 (d, 1H, J=12.1 Hz), 1.90-1.74 (m, 1H), 1.50-1.32 (m, 2H). Anal. Calcd. for C₂₃H₂₁ClF₂N₄O₃S.0.3 H₂O: C, 53.92; H, 4.25; N, 10.93; S, 6.26. Found: C, 53.63; H, 4.23; N, 10.85; S, 6.26.

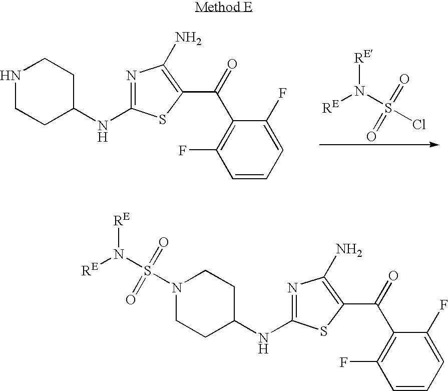

### Example E1

### 4-[4-Amino-5-(2,6-difluoro-benzoyl)-thiazol-2-ylamino]-piperidine-1-sulfonic acid dimethylamide

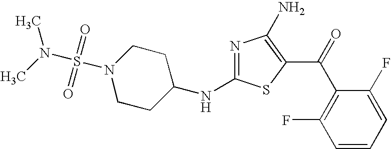

A solution of 1-[4-amino-2-(piperidin-4-ylamino)-thiazol-5-yl]-1-(2,6-difluoro-phenyl)-methanone (Example A6; 170 mg, 0.50 mmol) and dimethylsulfamoyl chloride (143 mg, 1.00 mmol) in pyridine was heated at 60° C. for 60 min. Pyridine was removed under reduced pressure and a solution of the resultant residue in ethyl acetate was washed with water, dried over MgSO₄, filtered, and concentrated. Purification via reversed phase preparative HPLC provided 150 mg of desired product in 70% yield.

¹H NMR (CD₃OD): δ 7.34 (m, 1H), 6.94 (m, 2H), 3.70 (br, 1H), 3.58 (m, 2H), 2.90 (m, 2H), 2.70 (s, 6H), 1.98 (m, 2H), 1.52 (m, 2H). HRMALDIMS. Calcd for C₁₇H₂₂F₂N₅O₃S₂ (MH⁺): 446.1132. Found: 446.1129.

### Example E2

### 4-[4-Amino-5-(2,6-difluoro-benzoyl)-thiazol-2-ylamino]-piperidine-1-sulfonic acid phenylamide

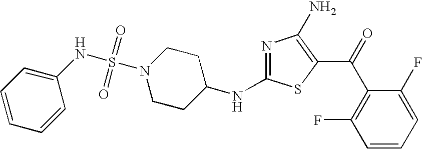

The title compound was prepared in a manner similar to that for Example E1 from 1-[4-amino-2-(piperidin-4-ylamino)-thiazol-5-yl]-1-(2,6-difluoro-phenyl)-methanone (Example A6) and phenylsulfamoyl chloride (Kloek, J. Org. Chem., Vol. 41, pp. 4028-4029 (1976)) to give a yellow foam in 31% yield.

¹H NMR (DMSO-d₆): δ 9.88 (s, 1H), 8.02 (bs, 2H), 7.52-7.42 (m, 1H), 7.28 (dd, 2H, J=7.3, 8.4 Hz), 7.20-7.10 (m, 3H), 7.02 (t, 1H, J=7.3 Hz), 3.54 (bd, 2H, J=13.1 Hz), 2.82 (dd, 2H, J=10.6, 11.5 Hz), 1.88 (d, 2H, J=9.5 Hz), 1.42-1.30 (m, 2H). HRMALDIMS. Calcd. for C₂₁H₂₂F₂N₅O₃S₂ (MH⁺): 494.1127. Found: 494.1118. Anal. Calcd. for C₂₁H₂₁F₂N₅O₃S₂.0.1 H₂O: C, 50.92; H, 4.31; N, 14,14; S, 12.95. Found: C, 50.80; H, 4.41; N, 13.83; S, 12.52.

### Example E3

### {4-Amino-2-[1-(4-methyl-piperazine-1-sulfonyl)-piperidin-4-ylamino]-thiazol-5-yl}-(2,6-difluoro-phenyl)-methanone

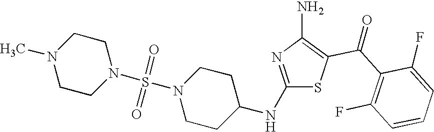

To a solution of 1-methyl-piperazine (2.0 g, 20 mmol) and diisopropylethylamine (5.2 g, 40 mmol) in CH₂Cl₂ at −30° C. was added chlorosulfonic acid (2.3 g, 20 mmol). After 2 hours at −30° C., the resultant suspension was filtered. The solid was thoroughly rinsed with CH₂Cl₂, dried under vacuum to give 2.2 g of 4-methyl-piperazine-1-sulfonic acid as an off white solid in 61% yield, which was used without further purification.

The above intermediate (1.79 g, 10.0 mmol) was placed in phosphorus oxychloride (50 ml). Phosphorous trichloride (6.2 g, 30 mmol) was added and heated at reflux for 3 hours. The solvent was removed under reduced pressure. A solution of the resultant residue in ethyl acetate was washed with sat. NaHCO₃, dried over MgSO₄, filtered, and concentrated to afford 1.5 g of 4-methyl-piperazine-1-sulfonyl chloride as a dark brown solid in 75% yield, which was used without further purification.

The title compound was prepared in a manner similar to that for Example E1 from 1-[4-amino-2-(piperidin-4-ylamino)-thiazol-5-yl]-1-(2,6-difluoro-phenyl)-methanone (Example A6) and 4-methyl-piperazine-1-sulfonyl chloride in 34% yield.

¹H NMR (CD₃OD): δ 7.38 (m, 1H), 6.92 (m, 2H), 3.70 (br, 1H), 3.58 (m, 2H), 3.18 (m, 4H), 2.92 (m, 2H), 2.40 (m, 4H), 1.96 (m, 2H), 1.50 (m, 2H). HRMALDIMS. Calcd for C₂₀H₂₇F₂N₆O₃S₂(MH⁺): 501.1554. Found: 501.1576

### Example E4

### 4-[4-Amino-5-(2,6-difluoro-benzoyl)-thiazol-2-ylamino]-piperidine-1-sulfonic acid amide

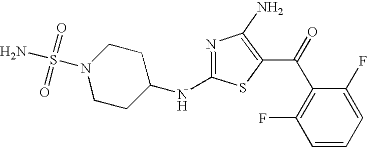

As described by Dewynter, et al., Tetrahedron, Vol. 49, pp. 65-76 (1993), to a solution of tert-butanol (2.0 ml, 21 mmol) in ethyl ether (20 ml) at −78° C., was added chlorosulfonyl isocyanate (0.40 ml, 4.6 mmol). The solution was allowed to warm to room temperature over 60 min. The solvent was removed under reduced pressure to give 0.82 g of N-carbamic acid t-butyl ester sulfonyl chloride as a clear oil in 95% yield, which was used immediately without further purification.

1-[4-Amino-2-(piperidin-4-ylamino)-thiazol-5-yl]-1-(2,6-difluoro-phenyl)-methanone (Example A6; 170 mg, 0.500 mmol) and above N-carbamic acid t-butyl ester sulfonyl chloride (187 mg, 1.00 mmol) was stirred in acetonitrile. After 60 min at room temperature, the solvent was removed in vacuo. A solution of the resultant residue in ethyl acetate was washed with 1% citric acid and sat. NaHCO₃, dried over MgSO₄, filtered, and concentrated to give 110 mg of yellow solid in 45% yield, which was used without further purification.

The above intermediate (0.10 g, 0.20 mmol) was dissolved in 30% TFA/CH₂Cl₂ and stirred for 30 minutes. The solvent was removed in vacuo. A solution of the resultant residue in ethyl acetate was washed with sat. NaHCO₃, dried over MgSO₄, filtered, and concentrated. The residue was triturated with ethyl ether and filtered off to give 75 mg of white powder in 90% yield.

¹H NMR (CD₃OD): δ 7.46 (m, 4H).7.08 m, (m, 1H).3.78, (m, 2H) 3.60, (m, 3H) 2.78 (m, 2H), 2.10 (m, 2H), 1.66 (m, 2H). HRMALDIMS. Calcd for C₁₅H₁₈F₂N₅O₃S₂(MH⁺): 418.0819. Found: 418.0831.

### Example E5

### [1-(4-{4-Amino-5-[1-(2,6-difluoro-phenyl)-methanoyl]-thiazol-2-ylamino}-piperidin-1-yl)-sulfonyl]-carbamic Acid Isopropyl Ester

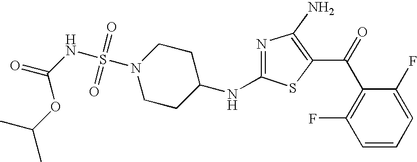

The title compound was prepared in a route with conditions similar to Example E4, except the reagent was prepared from isopropanol and chlorosulfonyl isocyanate instead.

¹H NMR (CD₃OD): δ 7.60m, 1H), 7.14 (m, 2H), 5.10 (q, 1H, J=5.4 Hz), 3.94 (m, 3H), 3.18 (m, 2H), 2.20 (m, 2H), 1.74 (m, 2H), 1.42 (d, 2H, J=5.4 Hz). LC-ESIMS (MH⁺): 504.

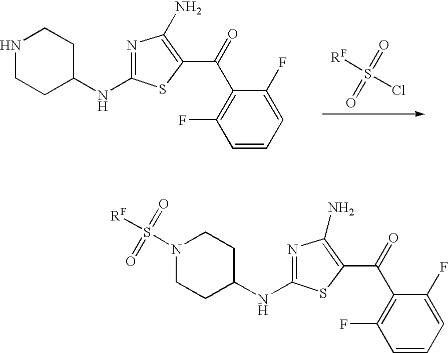

### Example F1

### 1-{4-Amino-2-[1-(3,5-dimethyl-isoxazole-4-sulfonyl)-piperidin4-ylamino]-thiazol-5-yl}-)1-(2,6-difluoro-phenyl)-methanone

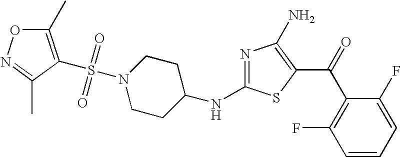

A solution of [4-amino-2-(piperidin-4-ylamino)-thiazol-5-yl]-(2,6-difluoro-phenyl)-methanone (Example A6; 47 mg, 0.14 mmol), 3,5-dimethylisoxazole4-sulfonyl chloride (33 mg, 0.17 mmol) and triethylamine (52 mg, 0.41 mmol) in acetonitrile (5 ml) stirred at room temperature for 2 hours. The reaction mixture was diluted with ethyl acetate. The resultant organic solution was washed with sat. NaHCO₃, dried over MgSO₄, filtered, and concentrated. The desired product was obtained in 55% yield after reversed phase HPLC purification.

¹H NMR (DMSO-d₆): δ 8.82 (br, 1H), 8.05 (s, 2H), 7.55-7.40 (m, 1H), 7.22-7.15 (m, 2H), 3.52-3.40 (m, 3H), 2.90-2.69 (m, 2H), 2.58 (s, 3H), 2.34(s, 3H), 2.07-1.86 (m, 2H), 1.58-1.39 (m, 2H). HRMALDIMS. Calcd for C₂₀H₂₁F₂N₅O₄S₂ (MH⁺): 498.1081. Found: 498.1087

In a manner similar to that for Example F1, the following Examples F2 to F18 were prepared from 1-[4-amino-2-(piperidin-4-ylamino)-thiazol-5-yl]-1-(2,6-difluoro-phenyl)-methanone (Example A6) and the corresponding commercially available sulfonyl chlorides.

### Example F2

### 1-{4-Amino-2-[1-(i-methyl-1H-imidazole-4-sulfonyl)-piperidin-4-ylamino]-thiazol-5-yl}-1-(2,6-difluoro-phenyl)-methanone

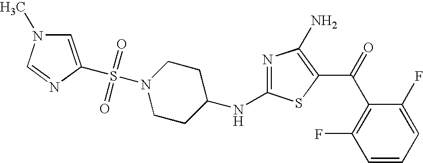

¹H NMR (DMSO-d₆/5% D₂O): δ 7.91-7.80 (m, 2H), 7.63-6.51 (m, 1H), 7.28-7.12 (m, 2H), 3.79 (s, 3H), 3.68-3.54 (m, 2H), 3.54-3.42 (m, 1H), 2.08-1.92 (m, 2H), 2.70-2.51 (m, 2H), 1.11-1.21 (m, 2H). HRMALDIMS. Calcd for C₁₉H₂₀F₂N₆O₃S₂Na (MNa⁺): 505.0904. Found: 505.0889

### Example F3

### 1-[4-Amino-2-(1-methanesulfonyl-piperidin-4-ylamino)-thiazol-5-yl]-1-(2,6-difluoro-phenyl)-methanone

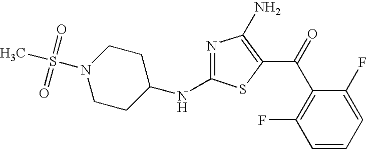

¹H NMR (DMSO-d₆): δ 8.78 (br, 1H), 8.02 (s, 2H), 7.52-7.29 (m, 1H), 7.19-7.08 (m, 2H), 3.52-3.38 (m, 3H), 2.90-2.74 (m, 2H), 2.83 (s, 3H), 1.99-1.88 (m, 2H), 1.57-1.41 (m, 2H). HRMALDIMS. Calcd for C₂₀H₂₂F₂N₅O₄S₂(MH⁺) 417.0867. Found: 417.0853

### Example F4

### 1-[4-Amino-2-(1-phenylmethanesulfonyl-piperidin-4-ylamino)-thiazol-5-yl]-1-(2,6-difluoro-phenyl)-methanone

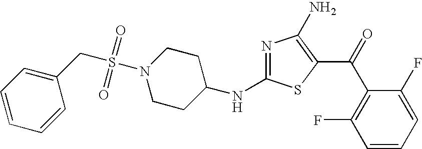

¹H NMR (DMSO-d₆): δ 8.75 (br, 1H), 8.02 (s, 2H), 7.59-7.45 (m, 1H), 7.45-7.32 (m, 5H), 7.23-7.11 (m, 2H), 4.39 (s, 2H), 3.53-3.42 (m, 3H), 2.92-2.77 (m, 2H), 1.98-1.83 (m, 2H), 1.50-1.33 (m, 2H). ESIMS (MH⁺): 536. Anal. Calcd for C₂₂H₂₂F₂N₄O₃S₂: C, 53.65; H, 4.50; N, 11.37; S, 13.02. Found: C, 53.76; H, 4.61; N, 11.14; S, 12.77.

### Example F5

### N-(4-{4-[4-Amino-5-(2,6-difluoro-benzoyl)-thiazol-2-ylamino]-piperidine-1-sulfonyl}-phenyl)-acetamide

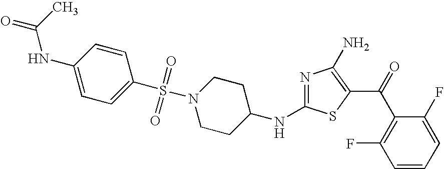

¹H NMR (DMSO-d₆): δ 8.65 (br, 1H), 7.97 (s, 1H), 7.99 (s, 2H), 7.80 (d, 2H, J=8.8 Hz), 7.65 (d, 2H, J=8.7 Hz), 7.53-7.42 (m, 1H), 7.19-7.07 (m, 2H), 3.48-3.34 (m, 3H), 2.56-2.44 (m, 2H), 2.10 (s, 3H) 1.97-1.86 (m, 2H), 1.58-1.42 (m, 2H). ESIMS (MH⁺): 493. Anal. Calcd for C₂₃H₂₃F₂N₅O₄S₂.0.3 Et₂O: C, 52.10; H, 4.70; N, 12.56; S, 11.50. Found: C, 52.09; H, 4.87; N, 12.27; S, 11.26.

### Example F6

### 1-{4-Amino-2-[1-(5-pyridin-2-yl-thiophene-2-sulfonyl)-piperidin-4-ylamino]-thiazol-5-yl}-1-(2,6-difluoro-phenyl)-methanone

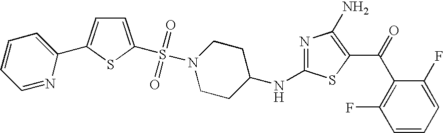

¹H NMR (DMSO-d₆/5% D₂O): δ 8.54 (d, 1H, J=4.2 Hz), 8.02-7.83 (m, 4H), 7.60 (d, 1H, J=4.0 Hz), 7.50-7.36 (m, 1H), 7.13-7.04 (m, 2H), 3.57-3.42 (m, 3H), 2.72-2.57 (m, 2H), 2.04-1.88 (m, 2H), 1.62-1.43 (m, 2H). Anal. Calcd for C₂₄H₂₁F₂N₅O₃S₃: C, 51.32; H, 3.77; N, 12.47; S, 17.13. Found: C, 51.07; H, 3.91; N, 12.20; S, 16.84.

### Example F7

### 1-{4-Amino-2-[1-(4-methoxy-benzenesulfonyl)-piperidin-4-ylamino]-thiazol-5-yl}-1-(2,6-difluoro-phenyl)-methanone

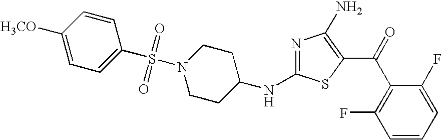

¹H NMR (DMSO-d₆): δ 8.72 (br, 1H), 7.98 (s, 2H), 7.68 (d, 2H, J=8.7 Hz), 7.53-7.42 (m, 1H), 7.19-7.10 (m, 4H), 3.83 (s, 3H), 3.48-3.34 (m, 3H), 2.58-2.40 (m, 2H), 1.98-1.85 (m, 2H), 1.59-1.42 (m, 2H). ESIMS (MH⁺): 509. Anal. Calcd for C₂₂H₂₂F₂N₄O₄S₂.0.8 Et₂O: C, 53.30; H, 5.33; N, 9.87; S, 11.29. Found: C, 53.15; H, 5.44; N, 9.73; S, 11.17.

### Example F8

### 1-{4-Amino-2-[1-(3,4-dimethoxy-benzenesulfonyl)-piperidin-4-ylamino]-thiazol-5-yl}-1-(2,6-difluoro-phenyl)-methanone

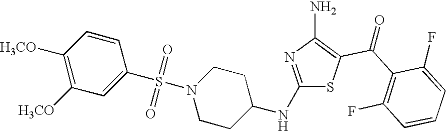

¹H NMR (DMSO-d₆): δ 8.74 (br, 1H), 7.99 (s, 2H), 7.52-7.43 (m, 1H), 7.38-7.23 (m, 1H), 7.20-7.11 (m, 4H), 3.85 (s, 3H), 3.83 (s, 3H), 3.50-3.42 (m, 3H), 2.59-2.43 (m, 2H), 1.98-1.87 (m, 2H), 1.58-1.44 (m, 2H). ESIMS (MH⁺): 539, (M−H⁻): 537. Anal. Calcd for C₂₃H₂₄F₂N₄O₅S₂: C, 51.29; H, 4.49; N, 10.40; S, 11.91. Found: C, 51.66; H, 4.73; N, 10.17; S, 11.66.

### Example F9

### 2-{4-[4-Amino-5-(2,6-difluoro-benzoyl)-thiazol-2-ylamino]-piperidine-1-sulfonyl}-benzonitrile

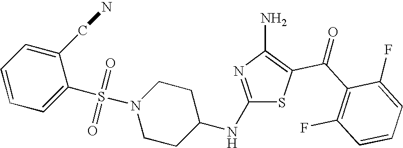

¹H NMR (DMSO-d₆): δ 8.83 (br, 1H), 8.29-8.14 (m, 1H), 8.13-7.96 (m, 3H), 7.63-7.52 (m, 1H), 7.27-7.17 (m, 2H), 3.74-3.66 (m, 3H), 3.02-2.86 (m, 2H), 2.10-2.00 (m, 2H), 1.67-1.52 (m, 2H). ESIMS (MH⁺): 504, (M−H⁻): 502. Anal. Calcd for C₂₂H₁₉F₂N₅O₃S₂.0.75 Et₂O: C, 53.70; H, 4.78; N, 12.73; S, 11.47. Found: C, 53.50; H, 4.93; N, 12.42; S, 11.44.

### Example F10

### 3-{4-[4-Amino-5-(2,6-difluoro-benzoyl)-thiazol-2-ylamino]-piperidine-1-sulfonyl}-thiophene-2-carboxylic acid methyl ester

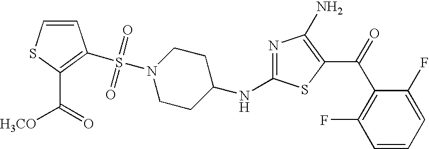

¹H NMR (DMSO-d₆): δ 8.90 (br, 1H), 8.21-8.09 (m, 1H), 7.63-7.48 (m, 2H), 7.27-7.12 (m, 2H), 3.99 (s, 3H), 3.84-3.70 (m, 3H), 3.12-2.98 (m, 2H), 2.10-1.88 (m, 2H), 1.57-1.42 (m, 2H). ESIMS (MH⁺): 543. Anal. Calcd for C₂₁H₂₀F₂N₄O₅S₃: C, 46.49; H, 3.72; N, 10.33; S, 17.73. Found: C, 46.73; H, 3.88; N, 10.12; S, 17.62.

### Example F11

### 1-{4-Amino-2-[1-(propane-2-sulfonyl)-piperidin-4-ylamino]-thiazol-5-yl}-1-(2,6-difluoro-phenyl)-methanone

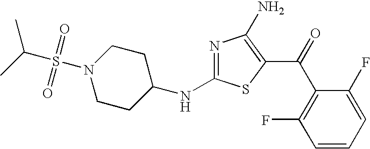

¹H NMR (DMSO-d₆): δ 8.75 (br, 1H), 8.00 (s, 2H), 7.52-7.37 (m, 1H), 7.18-7.04 (m, 2H), 3.60-3.42 (m, 3H), 3.00-2.97 (m, 3H), 1.98-1.79 (m, 2H), 1.48-1.30 (m, 2H), 1.20-1.09 (m, 6H). HRMALDIMS. Calcd for C₁₈H₂₃F₂N₄O₃S₂ (MH⁺): 445.1180. Found: 445.1186

### Example F12

### 1-{4-Amino-2-[1-(4-methanesulfonyl-benzenesulfonyl)-piperidin-4-ylamino]-thiazol-5-yl}-1-(2,6-difluoro-phenyl)-methanone

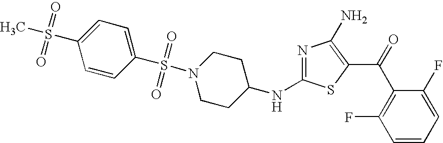

8.18 (d, 2H, J=8.5 Hz), 7.99 (d, 2H, J=8.5 Hz), 7.54-7.42 (m, 1H), 7.18-7.09 (m, 2H), 3.59-3.42 (¹H NMR (DMSO-d₆): δ m, 3H), 3.34 (s, 3H), 2.70-2.54 (m, 2H), 2.00-1.87 (m, 2H), 1.59-1.42 (m, 2H). ESIMS (MH⁺): 557. Anal. Calcd for C₂₂H₂₂F₂N₄O₅S₂: C, 47.47; H, 3.98; N, 10.07; S, 17.28. Found: C, 47.72; H, 4.16; N, 9.85; S, 17.06.

### Example F13

### 1-{4-Amino-2-[1-(2,5-dichloro-thiophene-3-sulfonyl)-piperidin-4-ylamino]-thiazol-5-yl}-1-(2,6-difluoro-phenyl)-methanone

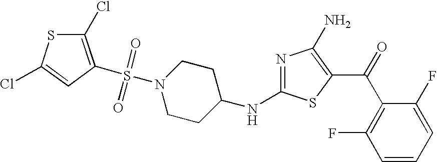

¹H NMR (DMSO-d₆): δ 8.73 (br, 1H), 7.97 (s, 2H), 7.50-7.38 (m, 1H), 7.33 (s, 1H), 7.17-7.04 (m, 2H), 3.58-3.47 (m, 3H), 2.88-2.75 (m, 2H), 1.98-1.84 (m, 2H), 1.53-1.36 (m, 2H). HRMALDIMS. Calcd for C₁₉H₁₇Cl₂F₂N₄O₃S₃ (MH⁺): 552.9808. Found: 552.9802

### Example F14

### 4-{4-[4-Amino-5-(2,6-difluoro-benzoyl)-thiazol-2-ylamino]-piperidine-1-sulfonyl}-benzoic Acid

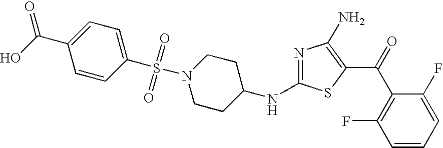

¹H NMR (DMSO-d₆): δ 8.74 (br.1H), 8.18 (d, 2H, J=7.8 Hz), 8.00 (br, 2H), 7.88 (d, 2H, J=7.8 Hz), 7.48 (m, 1H), 7.18 (m, 2H), 3.50 (m, 3H), 2.63 (m, 2H), 1.95 (m, 2H), 1.54 (m, 2H). HRMALDIMS. Calcd for C₂₂H₂₁F₂N₄O₅S₂ (MH⁺): 523.0916. Found: 523.0901

### Example F15

### {4-Amino-2-[1-(toluene-4-sulfonyl)-piperidin-4-ylamino]-thiazol-5-yl}-(2,6-difluoro-phenyl)-methanone

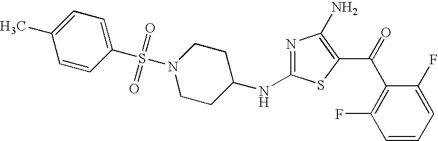

¹H NMR: δ 7.68 (d, 2H, J=8.2 Hz), 7.36 (d 2H, J=8.2 Hz), 7.30 (m, 1H), 6.94 (m, 2H), 3.70 (m, 2H), 3.38 (br, 1H), 2.46 (m, 2H; s, 3H), 2.10 (m, 2H), 1.62 (m, 2H). HRMALDIMS. Calcd for C₂₂H₂₃F₂N₄O₅S₂ (MH⁺): 493.1174. Found: 493.1185.

### Example F16

### 1-{4-Amino-2-[1-(5-bromo-6-chloro-pyridine-3-sulfonyl)-piperidin-4-ylamino]-thiazol-5-yl}-1-(2,6-difluoro-phenyl)-methanone

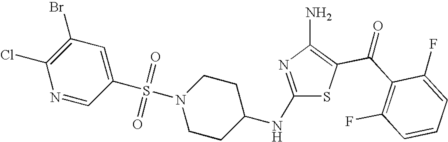

¹H NMR (DMSO-d₆): δ 8.76 (m, 1H), 8.75 (d, 1H, J=2.1 Hz), 8.52 (d, 1H, J=2.1 Hz), 7.98 (br, 2H), 7.54-7.42 (m, 1H), 7.15 (dd, 2H, J=7.8, 8.1 Hz), 3.59-3.50 (m, 2H), 3.35-3.23 (m, 1H), 2.80-2.64 (m, 2H), 2.00-1.88 (m, 2H), 1.59-1.42 (m, 2H). HRMALDIMS. Calcd. For C₂₀H₁₈BrClF₂N₅O₃S₂ (MH⁺): 591.9686. Found: 591.9664. Anal. Calcd. for C₂₀H₁₇BrClF₂N₅O₃S₂: C, 40.52; H, 2.89; N, 11.81; S 10.82. Found: C, 40.52; H, 3.00; N, 11.86; S, 10.78.

### Example F17

### 1-{4-Amino-2-[1-(4-fluoro-benzenesulfonyl)-piperidin-4-ylamino]-thiazol-5-yl}1-(2,6-difluoro-phenyl)-methanone

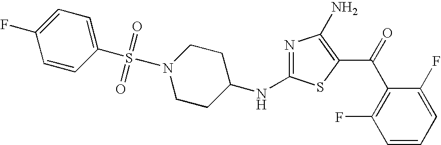

Obtained a yellow foam in 91% yield.

¹H NMR (CD₃OD): δ 7.84 (2H, ddd, J=2.0, 5.1, 7.0 Hz), 7.42 (1H, ddd, J=2.1, 6.4, 8.6 Hz), 7.33 (2H, dd, J=8.7, 8.8 Hz), 7.00 (2H, ddd, J=0.9, 3.2, 8.4 Hz), 3.62 (2H, bd, J=12.5 Hz), 2.54 (2H, ddd, J=2.7, 11.1, 13.7 Hz), 2.10-2.00 (2H, dd, J=3.7, 13.2 Hz), 1.64-1.52 (2H, m). ESIMS (MH⁺): 497. Anal. Calcd for C₂₁H₁₉F₃N₄O₃S₂: C, 50.80; H, 3.86; N, 11.28; S, 12.92. Found: C, 51.04; H, 4.04; N, 11.08; S, 12.68.

### Example F18

### 4-{4-[4-Amino-5-(2,6-difluoro-benzoyl)-thiazol-2-ylamino]-piperidine-1-sulfonyl}benzonitrile

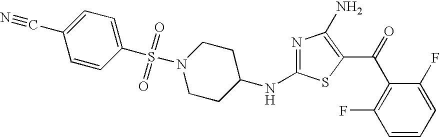

¹H NMR (CD₃OD): δ 7.80 (m, 4H), 7.22 (m, 1H), 6.84 (m, 2H), 3.48 (m, 3H), 2.44 (m, 2H), 1.88 (m, 2H), 1.40 (m, 2H). Anal. Calcd for C₂₂H₁₉F₂N₅O₃S₂: C, 52.48; H, 3.80; N, 13.91; S, 12.74. Found: C, 52.27; H, 3.89; N, 13.89; S, 12.64.

### Example F19

### 1-{4-Amino-2-[1-(6-dimethylamino-pyridine-3-sulfonyl)-piperidin-4-ylamino]-thiazol-5-yl}-1-(2,6-difluoro-phenyl)-methanone

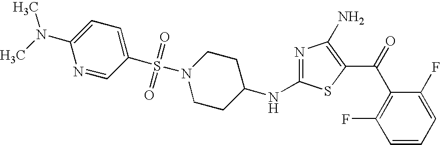

The starting materials were initially prepared along a typical route from literature, for example, Markley, et al., J. Med. Chem., 29, 427-433 (1986). Details are provided as follows:

A solution of 2-chloro-5-nitro-pyridine (3.17 g, 20.0 mmol) and aqueous dimethylamine (40%, 5 ml) in ethanol was refluxed for 4 hours. Solvent was removed and a solution of the resultant residue in ethyl acetate was washed with sat. NaHCO₃, dried over MgSO₄, filtered, and concentrated to give 3.2 g of dimethyl-(5-nitro-pyridin-2-yl)-amine as a yellow solid in 98% yield, which was used without further purification.

¹H NMR (CD₃OD): δ 8.98 (d, 1H, J=2.2 Hz); 8.12 (dd, 1H, J=2.2, 8.4 Hz), 6.4 (d, 1H, J=8.4 Hz), 3.2 (s, 6H).

The above intermediate was dissolved in 1% concentrated HCl/methanol (200 ml) and hydrogenated over 10% Pd/C (0.5 g) at 20 psi for 2 hours. The catalyst was removed by filtration. The filtrate was concentrated to give 3.7 g of N², N²-dimethyl-pyridine-2,5-diamine dihydrochloride as a yellow solid in 95% yield, which was used without further purification.

To a solution of above intermediate (2.09 g, 10.0 mmol) in acetic acid (12 ml) and concentrated HCl (2.34 ml) at 5° C., NaNO₂ (0.68 g 10 mmol) was added in small portions. The resulting diazonium salt solution was added slowly into a solution of acetic acid (7.5 ml), SO₂ (8.2 g), CuCl₂ (0.37 g), and water (0.5 ml) at 5° C. The mixture was allowed to warm to room temperature and stirred for another 90 minutes until gas evolution ceased. The solution was concentrated under reduced pressure and the residue was dried under vacuum to give the crude 2-dimethylamino-pyridine-5-sulfonyl chloride hydrochloride as a dark brown solid, which was used immediately in next step without further purification.

The title compound was prepared in a manner similar to that for Example F1 from 1-[4-amino-2-(piperidin-4-ylamino)-thiazol-5-yl]-1-(2,6-difluoro-phenyl)-methanone (Example A6) and 2-dimethylamino-pyridine-5-sulfonyl chloride hydrochloride.

¹H NMR (CD₃OD): δ 8.52 (d, 1H, J=2.3 Hz), 7.70 (dd, 1H, J=2.3, 8.3 Hz), 7.34 (m, 1H), 6.94 (m, 2H), 6.52 (d, 1H, J=8.3 Hz), 3.68 (m, 2H), 3.40 (br, 1H), 3.22 (s, 6H), 2.56 (m, 2H), 2.12 (m, 2H), 1.68 (m, 2H). HRMALDIMS. Calcd for C₂₂H₂₅F₂N₆O₃S₂ (MH⁺): 523.1392. Found: 523.1377.

### Example F20

### 1-{4-Amino-2-[1-(6-morpholin-4-yl-pyridine-3-sulfonyl)-piperidin-4-ylamino]-thiazol-5-yl}-1-(2,6-difluoro-phenyl)-methanone Hydrochloride

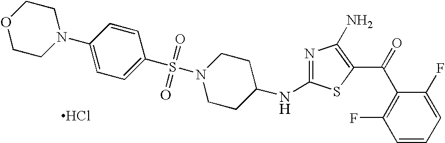

The starting material, 2-morpholin-4-yl-pyridine-5-sulfonyl chloride hydrochloride, was prepared in a route with conditions similar to that for 2-dimethylamino-pyridine-5-sulfonyl chloride in Example F19 from morpholine and 2-chloro-5-nitro-pyridine.

The title compound was prepared in a manner similar to that used to prepare the compound of Example F1 from 1-[4-amino-2-(piperidin-4-ylamino)-thiazol-5-yl]-1-(2,6-difluoro-phenyl)-methanone (Example A6) and 2-morpholin-4-yl-pyridine-5-sulfonyl chloride hydrochloride.

¹H NMR (CD₃OD): δ 8.38 (d, 1H, J=2.0 Hz), 8.08 (dd, 1H, J=2.0, 8.1 Hz), 7.64 (m, 1H), 7.30 (d, 1H, J=8.1 Hz), 3.88 (m, 4H), 3.80 (m, 4H), 3.70 (m, 3H), 2.76 (m, 2H), 2.12 (m, 2H), 1.70 (m, 2H). HRMALDIMS. Calcd for C₂₄H₂₆F₂N₆O₄S₂ (MH⁺): 565.1498. Found: 565.1481.

### Example F21

### 1-(4-Amino-2-[1-(6-chloro-pyridine-3-sulfonyl)-piperidin-4-ylamino]-thiazol-5-yl}-1-(2,6-difluoro-phenyl)-methanone

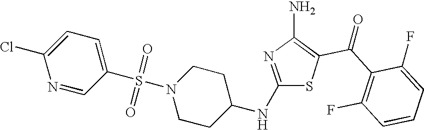 2-Chloro-pyridine-5sulfonyl Chloride Hydrochloride

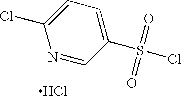

Initially prepared through a route with conditions similar to that for 2-dimethylamino-pyridine-5-sulfonyl chloride in Example F19, originating from 6-chloro-pyridin-3-ylamine. Subsequently available on multigram scale from German patent DE601896 (1934) and Naegeli, et al., Helv. Chim. Acta, Vol. 21, pp. 1746-1756 (1939).

¹H NMR: δ 9.03 (dd, 1H, J=0.5, 2.6 Hz), 8.25 (dd, 1H, J=2.6, 8.5 Hz), 7.61 (dd, 1H, J=0.5, 8.5 Hz).

The title compound was prepared in manner similar to that used to prepare the compound of Example F1 from 1-[4-amino-2-(piperidin-4-ylamino)-thiazol-5-yl]-1-(2,6-difluoro-phenyl)-methanone (Example A6) and 2-chloro-pyridine-5-sulfonyl chloride hydrochloride.

¹H NMR (DMSO-d₆): δ 8.78 (d, 1H, J=2.5 Hz), 8.20 (dd, 1H, J=2.6, 8.3 Hz), 7.81 (d, 1H, J=8.3 Hz), 7.56-7.44 (m, 1H), 7.22-7.12 (m, 2H), 3.60-3.38 (m, 3H), 2.81-2.61 (m, 2H), 1.98-1.83 (m, 2H), 1.52-1.36 (m, 2H). ESIMS (MH⁺): 514. Anal. Calcd for C₂₀H₁₈ClF₂N₅O₃S₂: C, 46.74; H, 3.53; N, 13.63; S, 12.48; Cl, 6.90. Found: C, 46.44; H, 3.56; N, 13.48; S, 12.41; Cl, 6.72.

### Example F22

### 1-{4-Amino-2-[1-(6-methoxy-pyridine-3-sulfonyl)-piperidin4-ylamino]-thiazol-5-yl}-1-(2,6-difluoro-phenyl)-methanone

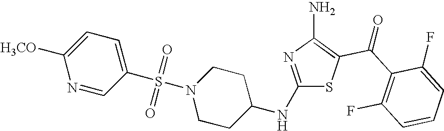

The starting material, 6-methoxy-pyridine-3-sulfonyl chloride was prepared in a manner similar to that for 2-dimethylamino-pyridine-5-sulfonyl chloride in Example F19 from 5-amino-2-methoxy-pyridine.

The title compound was prepared in a manner similar to that for Example F1 from 1-[4-amino-2-(piperidin-4-ylamino)-thiazol-5-yl]-1-(2,6-difluoro-phenyl)-methanone (Example A6) and 6-methoxy-pyridine-3-sulfonyl chloride.

¹H NMR (CD₃OD): δ 8.52 (s, 1H), 8.00 (br, 2H), 7.48 (m, 1H), 7.18 (m, 2H), 7.04 (d, 1H, J=8.0 Hz), 4.0 (s, 3H), 3.48 (m, 3H), 2.60 (m, 2H), 1.90 (m, 2H), 1.52 (m, 2H). HRMALDIMS. Calcd for C₂₁H₂₁F₂N₅O₄S₂Na (MNa⁺): 532.0895. Found: 532.0904.

### Example F23

### 1-{4-Amino-2-[1-(pyridine-3-sulfonyl)-piperidin-4-ylamino]-thiazol-5-yl}-1-(2,6-difluoro-phenyl)-methanone

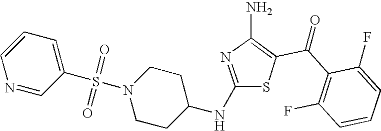

The title compound was prepared in manner similar to that for Example F1 from 1-[4-amino-2-(piperidin-4-ylamino)-thiazol-5-yl]-1-(2,6-difluoro-phenyl)-methanone (Example A6) and freshly prepared 3-pyridinesulfonyl chloride (Corey, et al, J. Org. Chem., 54, 389-393 (1989) and for NMR spectrum, see Karaman, et al J. Am. Chem. Soc., 114, 4889-4898 (1992)).

¹H NMR (DMSO-d₆): δ 8.84-7.73 (m, 2H), 8.68 (s, 1H), 8.13-8.04 (m, 1H), 7.92 (s, 2H), 7.66-7.54 (m, 1H), 7.43-7.29 (m, 1H), 7.12-6.94 (m, 2H), 3.49-3.28 (m, 3H), 3.63-3.42(m, 2H), 2.90-2.71 (m, 2H), 1.48-1.30 (m, 2H). HRMALDIMS. Calcd for C₂₀H₂₀F₂N₅O₃S₂ (MH⁺): 480.0976. Found: 480.0966

### Example F24

### 1-[4-Amino-2-{1-[4-(1-methyl-pyrrolidin-2-y)-benzenesulfonyl]-piperidin-4-ylamino}-thiazol-5-yl)-1-(2,6-difluoro-phenyl)-methanone Dihydrochloride

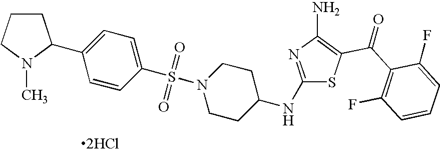

The starting material was prepared as follows:
1-Methyl-2-phenyl-pyrrolidine

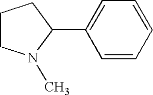

A solution of 2-phenylpyrrolidine (1.00 g, 6.79 mmol; Array Biopharma. Inc.) and paraformaldehyde (0.320 g, 10.7 mmol) in MeOH (15 ml) stirred at room temperature for 45 minutes. Sodium cyanoborohydride (0.70 g, 11 mmol) was added slowly, and the mixture then stirred for 12 hours. The solvent was removed under reduced pressure. A solution of the resultant residue in ethyl acetate was washed with sat. NaHCO₃, dried over MgSO₄, filtered, and concentrated. Purification via column chromatography (40% EtOAc/hexane) provided 0.45 g of an oil in 41% yield, which displayed a ¹H NMR spectrum that matched previous spectra (Lewis, et al J. Am. Chem. Soc., 113, 3498-3506 (1991)) and was used without further purification.

ESIMS (MH⁺): 162.

The title compound was prepared as follows. 1-Methyl-2-phenyl-pyrrolidine (0.45 g, 2.8 mmol) was cooled to 0° C. and chlorosulfonic acid (0.5 ml) was added slowly. The mixture was heated to 85° C. for 20 minutes, allowed to cool, and carefully quenched with cold water (30 ml). Solid Na₂CO₃was carefully added and the mixture was extracted with ethyl acetate. The extracts were dried over MgSO₄, filtered, and concentrated to give a thick oil, which was used in a manner similar to that for Example F1; with 1-[4-amino-2-(piperidin-4-ylamino)-thiazol-5-yl]-1-(2,6-difluoro-phenyl)-methanone (Example A6). The dihydrochloride salt was made as described in the general methods, from HPLC purification processing.

¹H NMR (CD₃OD): δ 8.02-7.83 (m, 3H), 7.82-7.73 (m, 1H), 7.54-7.42 (m, 1H), 7.12-7.02 (m, 2H), 4.58-4.47 (m, 1H), 3.97-3.86 (m, 1H), 3.78-3.65 (m, 3H), 3.40-3.32 (m, 1H), 2.87-2.83 (m, 3H), 2.70-2.56 (m, 3H), 2.43-2.27 (m, 3H), 2.17-2.04 (m, 2H), 1.73-1.59 (m, 2H). ESIMS (MH⁺): 562. Anal. Calcd for C₂₆H₂₉F₂N₅O₃S₂.2.0 HCl.0.75 H₂O: C, 48.18; H, 5.05; N, 10.81; S, 9.89. Found: C, 48.29; H, 5.25; N, 10.79; S, 9.46.

### Example F25

### 1-(4-Amino-2-[1-[4-(1-methyl-pyrrolidin-3-yl)-benzenesulfonyl]-piperidin-4-ylamino]-thiazol-5-yl}-1-(2,6-difluoro-phenyl)-methanone Dihydrochloride

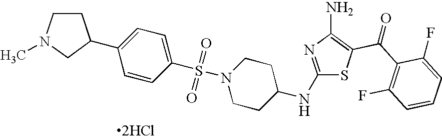

The starting materials were prepared as follows:
1-Methyl-3-phenyl-pyrrolidine.

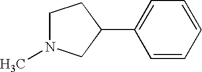

To a mixture of LiAlH₄ (1.00 g, 26.4 mmol) in dry THF (100 ml) at 0° C. was added 1-methyl-3-phenyl-pyrrolidine-2,5-dione (1.00 g, 5.28 mmol; U.S. Pat. No. 2,831,867). The resultant mixture was heated at reflux for 36 hours and allowed to cool to ambient temperature. Sodium sulfate decahydrate (1.9 g) was added carefully, followed by EtOAc (20 ml) and H₂O (0.6 ml). The mixture stirred for 5 hours at ambient temperature and filtered through a pad of Celite. The cake was washed with EtOAc and the filtrate concentrated in vacuo to give a yellow oil. Purification via column chromatography with 1% (58% NH₄OH)/10% MeOH/CHCl₃ as eluant afforded 0.59 g of yellow oil in 69% yield, which was used without any further purification.

¹H NMR: δ 7.36-7.24 (m, 4H), 7.23-7.16 (m, 1H), 3.40 (ddd, 1H, J=7.7, 9.7, 15.4 Hz), 3.02 (dd, 1H, J=8.6, 8.6 Hz), 2.82 (ddd, 1H, J=6.1, 7.9, 8.9 Hz), 2.65 (ddd, 1H, J=6.0, 8.8, 8.8 Hz), 2.50 (dd, 1H, J=8.1, 9.1 Hz), 2.42 (s, 3H), 2.38 (dddd, 1H, J=6.0, 7.8, 9.9, 13.0 Hz), 1.91 (dddd, 1H, J=6.0, 7.4, 8.5,13.0 Hz).
1-{4-Amino-2-[1-[4-(1-methyl-pyrrolidin-3-yl)-benzenesulfonyl]-piperidin-4-ylamino]-thiazol-5yl}-1-(2,6-difluoro-phenyl)-methanone

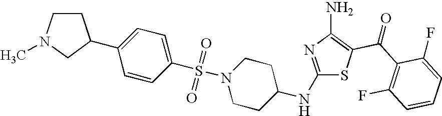

Chlorosulfonic acid (3 ml) was added dropwise to 1-methyl-2-phenyl-pyrrolidine (590 mg, 3.66 mmol) at 0° C. After 5 min, the resultant brown solution was heated at 95° C. for 1.5 hours, cooled to 0° C., and carefully poured into ice/H₂O. The aqueous solution was quickly extracted with CHCl₃ (3×25 ml). The combined organic layers were dried over Na₂SO₄, filtered, and concentrated in vacuo to afford 424 mg of a yellow gel (44% crude yield), which was immediately combined with 1-[4-amino-2-(piperidin-4-ylamino)-thiazol-5-yl]-1-(2,6-difluoro-phenyl)-methanone (Example A6) under conditions similar to that for Example F1, to provide 0.45 g of yellow foam in 59% yield.

¹H NMR (CD₃OD): δ 7.71 (d, 2H, J=8.4 Hz), 7.54 (d, 2H, J=8.3 Hz), 7.48-7.38 (m, 1H), 7.00 (dd, 2H, J=7.4, 7.5 Hz), 3.12 (dd, 1H, J=8.4, 9.5 Hz), 2.48 (s, 3H). ESIMS (MH⁺): 562. Anal. Calcd for C₂₆H₂₉F₂N₅O₃S₂.0.3 H₂O: C, 55.07; H, 5.26; N, 12.35; S, 11.31. Found: C, 55.08; H, 5.37; N, 11.98; S, 11.09.

The title compound was prepared as follows. To a solution of 1-(4-amino-2-[1-[4-(1-methyl-pyrrolidin-3-y)-benzenesulfonyl]-piperidin-4-ylamino]-thiazol-5-yl}-1-(2,6-difluoro-phenyl)-methanone (320 mg, 0.568 mmol) in MeOH (5 ml) was added a solution of HCl (0.355 ml of 4M in dioxane, 1.42 mmol). The solution was stirred for 30 min and concentrated in vacuo to afford 360 mg of yellow foam in 100% yield.

¹H NMR (CD₃OD): δ 7.74-7.65 (m, 2H), 7.55-7.47 (m, 2H), 7.44-7.32 (m, 2H), 7.00-6.91 (m, 2H), 3.98-3.66 (m, 3H), 3.65-3.50 (m, 4H), 3.48-3.30 (m, 2H), 2.97-2.91 (m, 3H), 2.58-2.40 (m, 3H), 2.00-1.91 (m, 2H), 1.60-1.43 (m, 2H). ESIMS (MH⁺): 562. Anal. Calcd for C₂₆H₂₉F₂N₅O₃S₂.2.1 HCl.1.0 H₂O: C, 47.58; H, 5.08; N, 10.67; S, 9.77. Found: C, 47.32; H, 5.13; N, 10.55; S, 9.49.

### Example F26

### {4-Amino-2-[1-(2-dimethylamino-ethanesulfonyl)-piperidin-4-ylamino]-thiazol-5-yl}-(2,6-difluoro-phenyl)-methanone

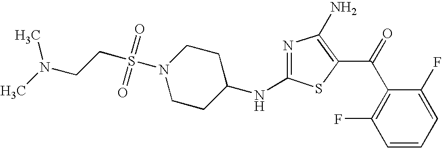

The title compound was prepared in manner similar to that used to prepare the compound of Example F1 from 1-[4-amino-2-(piperidin-4-ylamino)-thiazol-5-yl]-1-(2,6-difluoro-phenyl)-methanone (Example A6) and 2-dimethylamino-ethanesulfonyl chloride hydrochloride (Owens, et al., Eur. J. Med. Chem. Chim. Ther. 23, 295-300, (1988)).

¹H NMR (CD₃OD): δ 7.48 (m, 1H), 7.06 (m, 2H), 3.82 (m, 3H), 3.60 (m, 4H), 3.15 (m, 2H), 3.00 (s, 6H), 2.16 (m, 2H), 1.68 (m, 2H). HRMALDIMS. Calcd for C₁₉H₂₅F₂N₅O₃S₂ (MH⁺): 395.1717. Found: 395:1725.

### Example F27

### 1-{4-Amino-2-[1-(2-pyridin-4-yl-ethanesulfonyl)-piperidin-4-ylamino]-thiazol-5-yl}-1-(2,6-difluoro-phenyl)-methanone

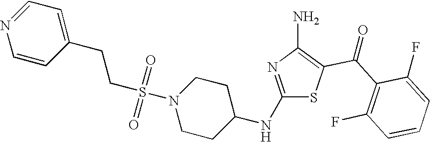

The starting material was prepared as outlined in Kempf, et al J. Med. Chem., Vol. 36, pp. 320-330 (1993).
2-Pyridin-4-yl-ethanesulfonyl Chloride Hydrochloride

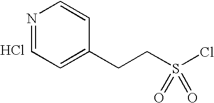

To a solution of 4-pyridineethanesulfonic acid in POCl₃ (6 ml), was added PCl₅ (0.75 g, 4.0 mmol). After heating at 60° C. for 2 hours, then cooled to 0° C., whereupon a solid was obtained, that was triturated with CCl₄, filtered, rinsed with CCl₄ and anhydrous ethyl ether, and dried under vacuum to give 1.51 g of yellow powder in 78% yield. Used crude without further characterization or purification.

¹H NMR (DMSO-d₆): δ 8.79 (d, 2H, J=6.7 Hz), 8.01 (d, 2H, J=6.7 Hz), 3.20 (t, 2H, J=7.6 Hz), 2.89 (t, 2H, J=7.6 Hz).

The title compound was prepared in manner similar to that used to prepare the compound of Example F1 from 1-[4-amino-2-(piperidin-4-ylamino)-thiazol-5-yl]-1-(2,6-difluoro-phenyl)-methanone (Example A6) and crude presumed 2-pyridin-4-yl-ethanesulfonyl chloride hydrochloride.

¹H NMR (DMSO-d₆): δ 8.37 (d, 2H, J=5.6 Hz), 7.92 (br, 2H), 7.37 (m, 1H), 7.22 (d, 1H, J=5.6 Hz), 7.04 (dd, 2H, J=8.1, 7.6 Hz), 3.50-3.40 (m, 2H), 3.32-3.23 (m, 2H), 3.15 (m, 1H), 2.92-2.80 (m, 4H), 1.89-1.78 (m, 2H), 1.43-1.28 (m, 2H). HRMALDIMS. Calcd. for C₂₂H₂₄F₂N₅O₃S₂ (MH⁺): 508.1283. Found: 508.1265. Anal. Calcd. for C₂₂H₂₃F₂N₅O₃S₂.0.5 H₂O: C, 51.15; H, 4.68; N, 13.56; S, 12.41. Found: C, 51.32; H, 4.62; N, 13.69; S 12.35.

### Example F28

### 1-{4-Amino-2-[1-(2-pyridin-2-yl-ethanesulfonyl)-piperidin-4-ylamino]-thiazol-5-yl}-1-(2,6-difluoro-phenyl)-methanone

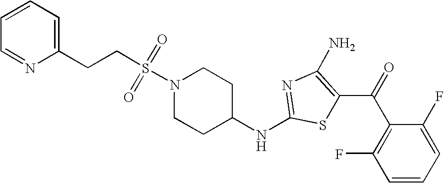

The starting material was prepared as described (Kempf, et al., J. Med. Chem., 36, 320-330 (1993)).
2-Pyridin-2-yl-ethanesulfonyl Chloride Hydrochloride

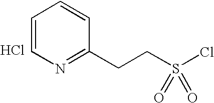

¹H NMR (DMSO-d₆): δ 8.50 (d, 1H, J=4.0 Hz), 7.73 (dd, 1H, J=1.9, 7.7 Hz), 7.49 (m, 1H), 7.37 (d, 1H, J=7.7 Hz), 3.20 (t, 2H, J=7.4 Hz), 2.89 (t, 2H, J=7.4 Hz).

The title compound was prepared in manner similar to that used to prepare the compound of Example F1 from 1-[4-amino-2-(piperidin-4-ylamino)-thiazol-5-yl]-1-(2,6-difluoro-phenyl)-methanone (Example A6) and 2-pyridin-2-yl-ethanesulfonyl chloride hydrochloride.

¹H NMR (DMSO-d₆): δ 8.80 (br, 1H), 8.50 (d, 1H, J=4.0 Hz), 8.05 (br, 2H), 7.73 (dd, 1H, J=1.9, 7.8 Hz), 7.49 (m, 1H), 7.37 (d, 1H, J=7.7 Hz), 7.26 (m, 1H), 7.16 (dd, 2H, J=7.7, 8.0 Hz), 3.60-3.51 (m, 2H), 3.44 (dd, 2H, J=5.1, 8.3 Hz), 3.13 (dd, 2H, J=5.1, 8.3, Hz), 2.96 (t, 2H, J=10.3 Hz), 2.00-1.89 (m, 2H), 1.48 (m, 2H). HRMALDIMS. Calcd. For C₂₂H₂₃F₂N₅O₃S₂ Na (MNa⁺): 530.1103. Found: 530.1098. Anal. Calcd. for C₂₂H₂₃F₂N₅O₃S₂.0.6 H₂O: C, 50.97; H, 4.71; N. 13.51; S, 12.37. Found: C, 51.08; H, 4.87; N, 13.29; S, 12.18.

### Example F29

### 1-{4-Amino-2-[1-(5-nitro-pyridine-2-sulfonyl)-piperidin-4-ylamino]-thiazol-5-yl}-1-(2,6-difluoro-phenyl)-methanone

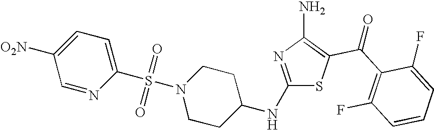

The title compound was prepared in manner similar to that for Example F1 from 1-[4-amino-2-(piperidin-4-ylamino)-thiazol-5-yl]-1-(2,6-difluoro-phenyl)-methanone (Example A6) and 5-nitro-pyridine-2-sulfonyl chloride hydrochloride (Caldwell et al., J. Amer. Chem. Soc., 66, 1479-1484, (1944)).

¹H NMR (CD₃OD): δ 9.60 (d, 1H, J=2.5 Hz), 8.88 (dd, 1H, J=2.5, 8.5 Hz), 8.28 (d, 1H, J=8.6 Hz), 7.56-7.42 (m, 1H), 7.10 (dd, 1H, J=7.5, 8.2 Hz), 3.10 (dd, 2H, J=10.8, 11.4 Hz), 2.18 (d, 2H, J=12.6 Hz), 1.80-1.62 (m, 2H). Anal. Calcd. for C₂₀H₁₈F₂N₆O₅S₂: C, 45.80; H, 3.46; N, 16.02; S, 12.23. Found: C, 45.78; H, 3.63; N, 15.91; S, 12.08. LC-ESIMS (M+H⁺): 525

### Example F30

### 1-(4-Amino-2-{1-[4-(1H-imidazol-4-y)-benzenesulfonyl]-piperidin-4-ylamino}-thiazol-5-yl)-1-(2,6-difluoro-phenyl)-methanone Trifluoroacetic Acid Salt

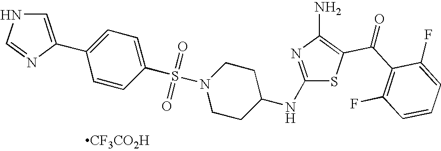

The starting materials were prepared as follows:
4-(1H-Imidazol-4-yl)-benzenesulfonic Acid

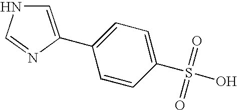

Following a procedure disclosed in U.S. Pat. No. 3,719,759 (Example 125), to 4-phenylimidazole (1.0 g, 6.9 mmol) was slowly added chlorosulfonic acid (2 ml). The mixture was heated at 95° C. overnight, allowed to cool to room temperature and carefully poured onto ice. The solid was collected by filtration and recrystallized from water to give 0.49 g of white powder in 32% yield, which was used without further purification.

¹H NMR (D₂O): δ 8.75 (d, 1H, J=1.4 Hz), 7.89 (dt, 1H, J=2.0, 8.7 Hz), 7.80 (d, 1H, J=1.4 Hz), 7.77 (dt, 1H, J=2.0, 8.7 Hz).

The title compound was prepared as follows. 4-(1H-lmidazol-4-yl)-benzenesulfonic acid (237 mg, 1.06 mmol) was placed in a flask and cooled to 0° C. Thionyl chloride (1.5 ml) was added under argon, followed with the addition of DMF (0.1 ml). The mixture stirred at 60° C. until the suspension became a clear solution (1 hour). Excess thionyl chloride was evaporated under reduced pressure. The residue was aezotroped with heptane twice and dried under vacuum to give a yellow solid, which was placed immediately with 1-[4-amino-2-(piperidin-4-ylamino)-thiazol-5-yl]-1-(2,6-difluoro-phenyl)-methanone (Example A6) under conditions similar to that for Example F1. Purification via preparative HPLC provided a white powder in 42% yield.

¹H NMR (CD₃OD): δ 9.27 (s 1H), 8.30 (s,1H), 8.18 (d, 2H, J=8.6 Hz), 8.13 (d, 2H, J=8.6 Hz), 7.62 (m, 1H), 7.20 (dd, 2H, J=7.5, 8.3 Hz), 3.99-3.82 (m, 3H), 2.92-2.75 (m, 2H), 2.35-2.23 (m, 2H), 1.91-1.75 (m, 2H). LCMS (MH⁺): 545. Anal. Calcd. for C₂₄H₂₂F₂N₆O₃S₂.1.8 TFA-1.0 H₂O: C, 43.17; H, 3.39; N, 10.94; S, 8.35. Found: C, 43.20; H, 3.30; N, 11.00; S, 8.48.

### Example F31

### 1-(4-Amino-2-{1-[4-(1-methyl-1H-imidazol-4-yl)-benzenesulfonyl]-piperidin-4-ylamino}-thiazol-5-yl)-1-(2,6-difluoro-phenyl)-methanone Trifluoroacetic Acid Salt

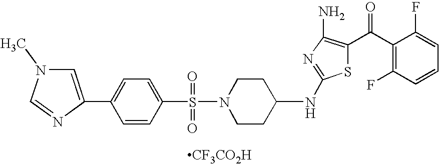

The starting material, 4-(1-methyl-1H-imidazol-4-yl)-benzenesulfonic acid, was prepared in a route similar to that of 4-(1H-imidazol4-yl)-benzenesulfonic acid in Example F30 from 1-methyl-4-phenyl-1H-imidazole (Kashima, et al, Heterocycles, Vol. 35, pp. 433-440 (1 993)).

The title compound was prepared in a manner similar to that used in preparation of Example F30 from 1-[4-amino-2-(piperidin-4-ylamino)-thiazol-5-yl]-1-(2,6-difluoro-phenyl)-methanone (Example A6) and 4-(1-methyl-1H-imidazol4-yl)-benzenesulfonic acid, and purification via preparative HPLC provided a white powder in 58% yield.

¹H NMR (DMSO-d₆): δ 8.63 (br, 2H), 8.10 (s, 1H), 7.92 (d, 4H, J=8.5 Hz), 7.75 (d, 2H, J=8.5 Hz), 7.40 (m, 1H), 7.06 (dd, 2H, J=7.6, 8.1 Hz), 3.78 (s, 3H), 3.48-3.38 (m, 2H), 2.58-2.43 (m, 2H), 1.92-1.78 (m, 2H), 1.52-1.35 (m, 2H). MS: (M+H⁺): 559. Anal. Calcd. for C₂₅H₂₄F₂N₆O₃S₂.1.5 TFA.2.5 H₂O: C, 43.92; H, 3.88; N, 10.98; S, 8.38. Found: C, 43.88; H, 4.02; N, 10.98; S, 8.34.

### Example F32

### 1-(4-Amino-2-{1-[4-(3-methyl-3H-imidazol-4-yl)-benzenesulfonyl]-piperidin-4-ylamino}-thiazol-5-yl)-1-(2,6-difluoro-phenyl)-methanone Trifluoroacetic Acid Salt

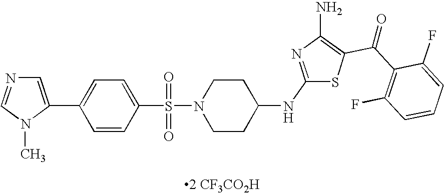

The starting material, 4-(3-methyl-3H-imidazol-4-yl)-benzenesulfonic acid, was prepared in a manner similar to that for 4-(1H-imidazol-4-yl)-benzenesulfonic acid in Example F30 from 1-methyl-5-phenyl-1H-imidazole (Kashima, et al., Heterocycles, Vol. 35, pp. 433-440 (1993)).

The title compound was prepared in a route similar to that for Example F30 from 1-[4-amino-2-(piperidin4-ylamino)-thiazol-5-yl]-1-(2,6-difluoro-phenyl)-methanone (Example A6) and 4-(3-methyl-3-H-imidazol-4-yl)-benzenesulfonic acid and subsequent purification via preparative HPLC provided a white powder in 52% yield.

¹H NMR (DMSO-d₆): δ 9.13 (s, 1H), 8.72 (br, 1H), 7.94-7.85 (m, 3H), 7.83 (d, 2H, J=8.5 Hz), 7.79 (d, 2H, J=8.5 Hz), 7.39 (m, 1H), 7.06 (dd, 2H, J=7.6, 8.2 Hz), 3.81 (s, 3H), 3.52-3.43 (m, 2H), 2.62-2.45 (m, 2H), 1.92-1.80 (m, 2H), 1.53-1.37 (m, 2H). LCMS(MH⁺): 559. Anal. Calcd. for C₂₅H₂₄F₂N₆O₃S₂.2.0 TFA.1.0 H₂O: C, 43.29; H, 3.51; N, 10.44; S, 7.97. Found: C, 43.12; H, 3.72; N, 10.56; S, 7.90.

### Example F33

### 1-(4-Amino-2-{1-[4-(2-methyl-1H-imidazol-4-yl)-benzenesulfonyl]-piperidin-4-ylamino}-thiazol-5-yl)-1-(2,6-difluoro-phenyl)-methanone Dihydrochloride

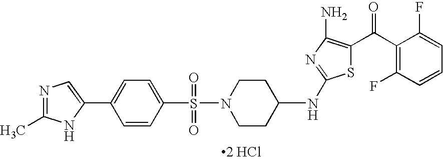

The starting materials were prepared as follows:
4-Phenyl-1-triphenylmethyl-1H-imidazole

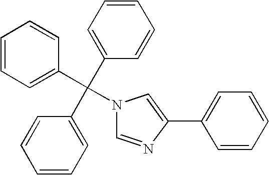

To a solution of 4-phenylimidazole (5.00 g, 34.7 mmol) and triethylamine (5.30 ml, 38.2 mmol) in DMF (50 ml) at 0° C., was added triphenylmethyl chloride (10.2 g, 36.4 mmol). The solution stirred at room temperature for 1.5 hours, then diluted with cold water (500 ml) to give a suspension. The white solid was collected by filtration, washed with water, and dried under vacuum to give 13.2 g of white powder in 98% yield, which was used without further purification.

¹H NMR: δ 7.73 (dd, 2H, J=1.4, 8.5 Hz), 7.49 (d, 1H, J=1.4 Hz), 7.38-7.28 (m, 11H), 7.24-7.18 (m, 7H), 7.12 (d, 1H, J=1.4 Hz).
2-Methyl-4-phenyl-1-triphenylmethyl-1H-imidazole

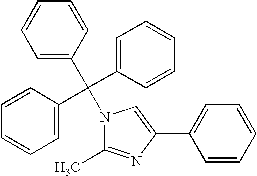

To a solution of4-phenyl-1-triphenylmethyl-1H-imidazole (3.86 g, 10.0 mmol) in THF (80 ml) at −78° C. under argon was added n-butyllithium (4.4 ml of 2.5 M in hexane). The resultant pink solution stirred at −78° C. for one hour, then iodomethane (4.5 g, 30 mmol) was added. After another hour, quenched with diethylamine (5 ml), and the solvent was removed in vacuo. The resultant residue was dissolved in ethyl ether, washed with sat. NaHCO₃, dried over Na₂SO₄, filtered, and concentrated to give 3.1 g of a white solid in 78% yield, which was used without further purification.

¹H NMR: δ 7.73 (dd, 2H, J=1.4, 8.5 Hz), 7.40-7.28 (m, 11H), 7.24-7.16 (m, 7H), 7.02 (s, 1H), 1.72 (s, 3H).
4-(2-Methyl-3H-imidazol-4-yl)-benzenesulfonic Acid

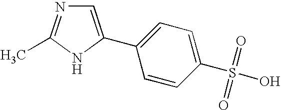

Prepared in a manner analogous to that for 4-(1H-imidazol-4-yl)-benzenesulfonic acid in Example F30. 2-Methyl-4-phenyl-1-triphenylmethyl-1H-imidazole (1.8 g, 4.5 mmol) and chlorosulfonic acid (2.5 ml) gave 546 mg (51% yield) of brown needles, which were used without further purification.

NMR (DMSO-d₆): δ 14.22 (b, 2H), 8.05 (s, 1H), 7.77 (d, 2H, J=8.8 Hz), 7.72(d, 2H, J=8.8 Hz), 2.64 (s, 3H).

The title compound was prepared in a route with conditions similar to that for Example F30 from 1-[4-amino-2-(piperidin-4-ylamino)-thiazol-5-yl]-1-(2,6-difluoro-phenyl)-methanone (Example A6) and 4-(2-methyl-3H-imidazol-4-yl)-benzenesulfonic acid to provide a white powder in 62% yield.

¹H NMR (DMSO-d₆): δ 14.80 (br, 1H), 14.30 (br, 1H), 8.67 (br, 1H), 8.10 (s, 1H), 7.94 (d, 2H, J=8.5 Hz), 7.85 (br, 1H), 7.76 (d, 2H, J=8.5 Hz), 7.34 (m, 1H), 7.00 (dd, 2H, J=7.7, 7.9 Hz), 3.45-3.32 (m, 3H), 2.53 (s, 3H), 2.50-2.40 (m, 2H), 1.87-1.76 (m, 2H), 1.47-1.33 (m, 2H). LCMS: (MH⁺): 559. Anal. Calcd. for C₂₅H₂₄F₂N₆O₃S₂.2.5 HCl.1.2 H₂O: C, 44.72; H, 4.34; N, 12.52; S, 9.55. Found: C, 44.71; H, 4.64; N, 12.43; S, 9.78.

### Example F34

### 1-(4-Amino-5-{1-[4-(1H-imidazol-2-yl)-benzenesulfonyl]-piperidin-4-ylamino}-thiazol-2-yl)-1-(2,6-difluoro-phenyl)-methanone

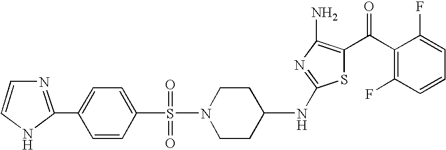

The title compound was prepared in manner similar to that for Example F1. 1-[4-Amino-2-(piperidin-4-ylamino)-thiazol-5-yl]-1-(2,6-difluoro-phenyl)-methanone (Example A6) and 4-(1H-imidazol-2-yl)-benzenesulfonyl chloride hydrochloride (based on a procedure in U.S. Pat. No. 3,719,759; Example 125) provided a yellow foam in 17% yield (over two steps, from 2-phenylimidazole).

¹H NMR (DMSO-d₆): δ 8.08 (d, 2H, J=8.6 Hz), 7.87 (d, 2H, J=8.6 Hz), 7.43 (ddd, 1H, J=2.2, 8.4, 12.6 Hz), 7.28-7.20 (m, 2H), 7.00 (dd, 2H, J=7.4, 8. 3Hz), 3.74-3.62 (m, 2H), 2.70-2.58 (m, 2H), 1.70-1.58 (m, 2H). Anal. Calcd. for C₂₄H₂₂F₂N₆O₃S₂.1.0 H₂O: C, 51.24; H, 4.30; N, 14.94; S, 11.40. Found: C, 50.88; H, 4.32; N, 14.55; S, 11.21.

### Example F35

### 4-{3-[4-Amino-5-(2,6-difluoro-benzoyl)-thiazol-2-ylamino]-piperidine-1-sulfonyl}-benzonitrile

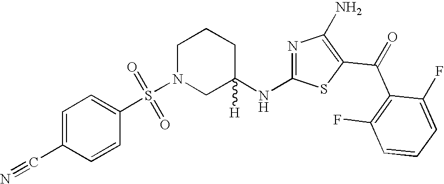

The title compound was prepared in a manner similar to that for Example F1. 1-[4-Amino-2-(piperidin-3-ylamino)-thiazol-5-yl]-1-(2,6-difluoro-phenyl)-methanone (Example A8) and 4-cyano-benzenesulfonyl chloride (Maybridge) gave a yellow foam in 67% yield.

¹H NMR (DMSO-d₆): δ 8.02 (d, 2H, J=8.4 Hz), 7.86 (d, 2H, J=8.5 Hz), 7.50-7.38 (m, 1H,), 7.10 (dd, 2H, J=7.8, 8.0 Hz), 3.48-3.42 (m, 1H), 1.78-1,64 (m, 2H), 1.52-1.20 (m, 2H). Anal. Calcd. for C₂₂H₁₉F₂N₅O₃S₂.0.45 CHCl₃: C, 48.39; H, 3.52; N, 12.57; S, 11.51. Found: C, 48.36; H, 3.69; N, 12.37; S, 11.55.

### Example F36

### N-(4-{3-[4-Amino-5-(2,6-difluoro-benzoyl)-thiazol-2-ylamino]-piperidine-1-sulfonyl}-phenyl)-acetamide

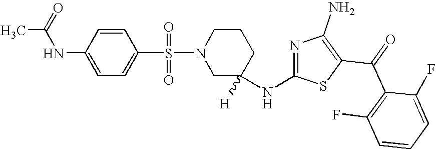

The title compound was prepared in a manner similar to that for Example F1. 1-[4-Amino-2-(piperidin-3-ylamino)-thiazol-5-yl]-1-(2,6-difluoro-phenyl)-methanone (Example A8) and 4-acetylamino-benzenesulfonyl chloride provided a yellow foam in 68% yield.

¹H NMR (DMSO-d₆): δ 8.10 (bs, 2H), 7.78 (d, 2H, J=8.8 Hz), 7.68 (d, 2H, J=8.8 Hz), 7.55-7.45 (m, 1H), 7.15 (dd, 2H, J=7.8, 15.8 Hz), 3.50-3.42 (m, 1H), 2.08 (s, 3H), 1.82-1.72 (m, 2H), 1.60-1.44 (m, 1H), 1.36-1.20 (m, 1H). Anal. Calcd. for C₂₃H₂₃F₂N₅O₄S₂.0.45 CHCl₃: C, 47.79; H, 4.01; N, 11.88; S, 10.88. Found: C, 47.84; H, 4.29; N, 11.90; S, 10.69.

### Example F37

### [4-Amino-2-(1-methanesulfonyl-piperidin-3-ylamino)-thiazol-5-yl]-(2,6-difluoro-phenyl)-methanone

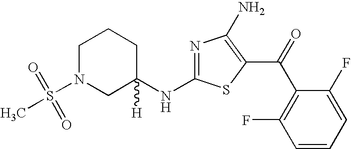

The title compound was prepared in a manner similar to that for Example F1 from 1-[4-amino-2-(piperidin-3-ylamino)-thiazol-5-yl]-1-(2,6-difluoro-phenyl)-methanone (Example A8) and methanesulfonyl chloride. Purified via preparative TLC (2 mm) with 8% MeOH/CH₂Cl₂ to afford a yellow solid in 68% yield.

¹H NMR (DMSO-d₆): δ 8.08 (bs, 2H), 7.50 (ddd, 1H, J=1.4, 7.1, 8.2 Hz), 7.16 (dd, 2H, J=7.7, 15.8 Hz), 3.52 (dd, 1H, J=3.6, 11;2 Hz), 2.88 (s, 3H), 2.78-2.70 (m, 1H), 1.92-1.76 (m, 2H), 1.58-1.42 (m, 2H). Anal. Calcd. for C₁₆H₁₈F₂N₄O₃S₂.0.6 MeOH: C, 45.76; H, 4.72; N, 12.86; S, 14.72. Found: C, 45.70; H, 4.64; N, 12.74; S, 14.32.

### Example F38

### 4-{3-[4-Amino-5-(2,6-difluoro-benzoyl)-thiazol-2-ylamino]-pyrrolidine-1-sulfonyl}-benzonitrile

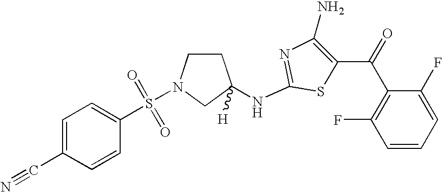

The title compound was prepared in a manner similar to that for Example F1. 1-[4-Amino-2-(pyrrolidin-3-ylamino)-thiazol-5-yl]-1-(2,6-difluoro-phenyl)-methanone (Example A10) and 4-cyano-benzenesulfonyl chloride provided 220 mg of yellow powder in 88% yield.

¹H NMR (DMSO-d₆): δ 8.80 (br, 1H), 8.13 (d, 2H, J=8.4 Hz), 8.01 (d, 2H, J=8.4 Hz), 7.57 (m, 1H), 7.22 (t, 2H, J=8.1 Hz), 4.17 (m, 1H), 3.53 (dd, 1H, J=5.7, 10.6, Hz), 3.42-3.24 (m, 3H), 2.13 (m, 1H), 1.86 (m, 1H). HRFABMS. Calcd. For C₂₁H₁₈F₂N₅O₃S₂ (MH⁺): 489.0741. Found: 489.0774. Anal. Calcd. for C₂₁H₁₇F₂N₅O₃S₂.0.1 hexane: C, 52.12; H, 3.65; N, 14.07; S, 12.88. Found: C, 51.93; H, 3.71; N, 13.91; S, 12.84.

### Example F39

### [4-Amino-2-(1-methanesulfonyl-pyrrolidin-3-ylamino)-thiazol-5-yl]-(2,6-difluoro-phenyl)-methanone

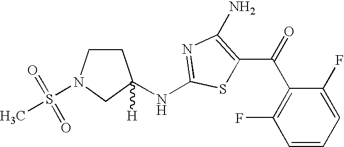

The title compound was prepared in a manner similar to that for Example F1. 1-[4-Amino-2-(pyrrolidin-3-ylamino)-thiazol-5-yl]-1-(2,6-difluoro-phenyl)-methanone (Example A10) and methanesulfonyl chloride provided 120 mg of yellow powder in 46% yield.

¹H NMR (DMSO-d₆): δ 8.99 (bd, 1H), 8.08 (bd, 2H), 7.51 (m, 1H), 7.17 (dd, 2H, J=7.8, 8.0 Hz), 4.26 (m, 1H), 3.54 (dd, 1H, J=6.1, 10.5 Hz), 3.39-3.27 (m, 5H), 3.16 (m, 1H), 2.21 (m, 1H), 1.92 (m, 1H). HRFABMS. Calcd. for C₁₅H₁₈F₂N₄O₃S₂ (MH⁺): 403.0705. Found: 403.0724. Anal. Calcd. for C₂₁H₁₇F₂N₅O₃S₂.0.2 CH₃OH.1.0 H₂O: C, 42.77; H, 4.44; N, 13.13; S, 15.02. Found: C, 42.66; H, 4.18; N, 12.79; S, 14.82.

### Example F40

### 4-{3S-[4-Amino-5-(2,6-difluoro-benzoyl)-thiazol-2-ylamino]-pyrrolidine-1-sulfonyl}-benzonitrile

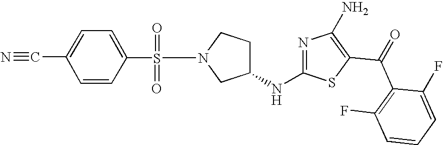

The title compound was prepared in a manner similar to that for Example F1. 4-Cyano-benzenesulfonyl chloride and 1-[4-amino-2-(pyrrolidin-3S-ylamino)-thiazol-5-yl]-1-(2,6-difluoro-phenyl)-methanone (Example A11) provided 288 mg of yellow powder in 95% yield, which displayed a ¹H NMR that matched Example F38.

HRFABMS. Calcd. for C₂₁H₁₈F₂N₅O₃S₂ (MH⁺): 490.0814. Found: 490.0896. Anal. Calcd. for C₂₁H₁₇F₂N₅O₃S₂.0.8 CH₃OH: C, 50.83; H, 3.95; N, 13.59; S, 12.45. Found: C, 50.59; H, 3.88; N, 13.36; S, 12.60.

### Example F41

### [4-3S-Amino-2-(1-methanesulfonyl-pyrrolidin-3-ylamino)-thiazol-5-yl]-(2,6-difluoro-phenyl)-methanone

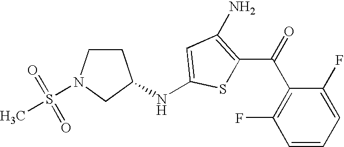

The title compound was prepared in a manner similar to that for Example F1 from methanesulfonyl chloride and 1-[4-amino-2-(pyrrolidin-3S-ylamino)-thiazol-5-yl]-1-(2,6-difluoro-phenyl)-methanone (Example A11) provided 138 mg of yellow powder in 53% yield, which displayed a ¹H NMR spectrum that matched Example F39.

HRFABMS. Calcd. for C₁₅H₁₈F₂N₄O₃S₂ (MH⁺): 403.0705. Found: 403.0719. Anal. Calcd. for C₂₁H₁₇F₂N₅O₃S₂.0.3 CH₃OH: C, 44.60; H, 4.21; N, 13.60; S, 15.56. Found: C, 44.45; H, 4.16; N, 13.50; S, 15.48.

### Example F42

### 1-{4-Amino-2-[1-(4-iodo-benzenesulfonyl)-piperidin-4-ylamino]-thiazol-5-yl}-1-(2,6-difluoro-phenyl)-methanone

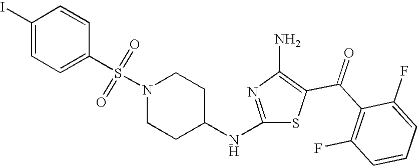

The title compound was prepared in a manner similar to that for Example F1. 1-[4-Amino-2-(piperidin4-ylamino)-thiazol-5-yl]-1-(2,6-difluoro-phenyl)-methanone (Example A6) and pipsyl chloride gave 1.70 g of a yellow powder in 95% yield, which was used without further characterization or purification.

¹H NMR (DMSO-d₆): δ 9.56 (br, 1H), 8.84 (b, 1H), 8.08 (d, 2H, J=8.3 Hz), 8.04 (br, 2H), 7.54 (d, 2H, J=8.3 Hz), 7.52 (m, 1H), 7.20 (dd, 2H, J=7.8, 7.9 Hz), 3.51-3.44 (m, 2H), 2.68-2.52 (m, 2H), 2.03-1.90 (m, 2H), 1.64-1.50 (m, 2H). LC-ESIMS (MH⁺): 605

### Example F43

### 4-{4-[4-Amino-5-[1-(2,6-difluoro-benzoyl)-thiazol-2-ylamino]-piperidine-1-sulfonyl}-benzaldehyde

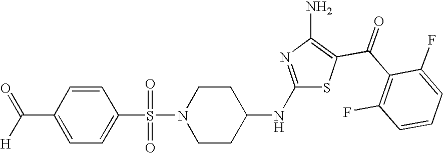

The title compound was prepared in a manner similar to that for Example F1 from 1-[4-amino-2-(piperidin-4-ylamino)-thiazol-5-yl]-1-(2,6-difluoro-phenyl)-methanone (Example A6) and 4-formyl-benzenesulfonyl chloride (AstaTech, Inc.). Used without further characterization or purification.

¹H NMR (CD₃OD): δ 8.78-8.59 (m, 4H), 8.39-8.23 (m, 1H), 7.97-7.82 (m, 2H), 3.62-3.43 (m, 3H), 2.53-2.34 (m, 2H), 1.98-1.86 (m, 2H), 1.57-1.40 (m, 2H). LC-ESIMS (MH⁺): 507.

### Example F44

### 1-{4-Amino-2-[1-(3-chloropropane-1-sulfonyl)-piperidin-4-ylamino]-thiazol-5-yl}-1-(2,6-difluoro-phenyl)-methanone

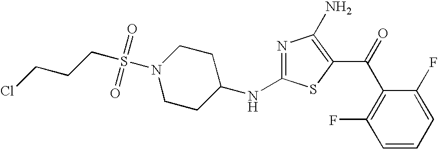

The title compound was prepared as follows. To a stirring solution of 1-[4-amino-2-(piperidin-4-ylamino)-thiazol-5-yl]-1-(2,6-difluoro-phenyl)-methanone (Example A6; 1.4 g, 4.1 mmol) in DMF were sequentially added diisopropylethylamine (3 ml) and 3-chloropropylsulfonylchloride (0.90 g, 5.0 mmol). After 2 hours the resultant mixture was poured into water (800 ml). The solids were filtered off and the resultant cake was washed with water and diethyl ether and dried to give 1.3 g of a white solid in 67% yield.

¹H NMR (DMSO-d₆): δ 8.78 (br, 1H), 8.04 (s, 2H), 7.50 (tt, 1H, J=4.6, 8.3 Hz), 7.14 (dd, 2H, J=7.7, 8.3 Hz), 3.73 (t, 2H, J=6.5 Hz), 3.55 (m, 2H), 3.14 (t, 2H, J=7.5 Hz,), 2.10 (tt, 2H, J=6.5, 7.5 Hz), 1.90 (m, 2H), 1.50 (m, 2H). Anal. For C₁₈H₂₁ClF₂N₄O₃S₂: C, H, N.

### Example F45

### 1-{4-Amino-2-[1-(3-iodopropane-1-sulfonyl)-piperidin-4-ylamino]-thiazol-5-yl}-1-(2,6-difluoro-phenyl)-methanone

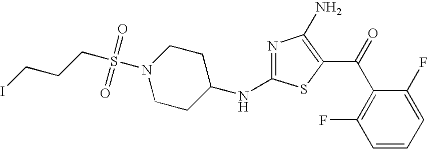

The title compound was prepared as follows. To a stirring solution of 1-{4-amino-2-[1-(3-chloropropane-1-sulfonyl)-piperidin-4-ylamino]-thiazol-5-yl}-1-(2,6-difluoro-phenyl)-methanone (Example F44; 6.00 g, 12.5 mmol) in acetone (100 ml) was added NaI (10 g) and heated to reflux. After 16 hours, the mixture was poured into water (800 ml) and extracted with EtOAc. The organic layer was dried over Na₂SO₄ and concentrated in vacuo to provide 6.4 g of a yellow solid in 90% yield, which was used without further purification.

¹H NMR (DMSO-d₆) δ: 8.79 bs, [symbol] ), 8.03 (s, 2H), 7.48 (tt, 1H, J=6.8, 8.2Hz), 7.15 (dd, 2H, J=7.6, 8.2Hz), 3.59-3.46 (m, 3H), 3.32 (t, 2H, J=7.0Hz), 3.10 (t, 2H, J=7.4Hz), 3.03-2.89 (m, 2H), 2.14 (tt, 2H, J=7.0, 7.4Hz), 2.01-1.86 (m, 2H), 1.56-1.38 (m, 2H). LC-ESIMS (MH⁺): 571

### Example F46

### 3-(4-{4-[4-Amino-5-(2,6-difluoro-benzoyl-2-ylamino]-piperidine-1-sulfonyl}-phenyl)-propionic acid methyl ester

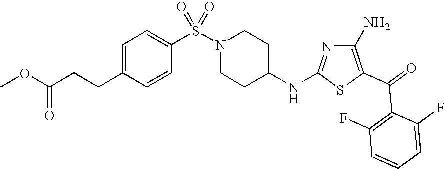

The title compound was prepared in a manner analogous to that used in Example F1. Methyl-3-(4-chlorosulphonyl) phenylpropionate and 1-[4-amino-2-(piperidine-4-ylamino)-thiazol-5-yl]-1-(2,6-difluoro-phenyl)-methanone (Example A6) gave, after recrystallization from Et₂O, a yellow solid in 74% yield.

¹H NMR (DMSO-d₆): δ 8.72 (bs, 1H), 8.05 (bs, 1H), 7.64 (d, 2H, J=8.0 Hz), 7.56-7.42 (m, 3H), 7.15 (t, 2H, J-15.9 Hz), 3.6 (s, 3H), 3.52-3.41 (m, 3H), 2.95 (t, 2H, J=7.6 Hz), 2.70 (t, 2H, J=7.6 Hz), 2.42-2.35 (m, 2H), 1.98-1.83 (m, 2H), 1.60-1.43 (m, 2H). HRMALDIMS: C₂₅H₂₇F₂N₄O₅S₂ (MH⁺): 565.1391. Found: 565.1387. Anal. Calcd. For C₂₅H₂₆F₂N₄O₅S₂: C, 53.18; H, 4.64; N, 9.92; S, 11.36. Found: C, 53.03; H, 4.85; N, 9.93; S, 11.30.

### Example F47

### (4-Amino-2-{1-[2-(4-methyl-piperazin-1-yl)-pyrimindin-5-sulfonyl]-piperidin-4-ylamino}-thiazol-5-yl)-(2,6-difluoro-phenyl)-methanone

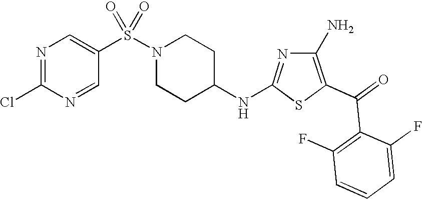

The starting materials of the title compound were prepared as follows:
2-Amino-5 pyrimidinesulfonic Acid.

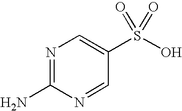

Slight modifications of the procedure from Caldwell et al, J. Amer. Chem. Soc, 81, 5166-5167 (1959) were used. To 40 ml of fuming sulfuric acid (20% free SO₃) was added cautiously 2-aminopyrimidine (9.5 g, 100 mmol). The temperature was then raised to 180° C. and kept there for five hours. After cooling, the contents of the flask were poured upon 400 g of crushed ice and lyophilized. The resulting solid was collected by filtration, washed with water, dried over P₂O₅ in vacuum to afford 3.26 g of a brown solid in 18% yield, which was used without further purification. Anal. Calcd. For C₄H₅N₃O₃S: C, 27.43; H, 2.88; N, 23.99; S, 18.31. Found: C, 27.47; H, 2.95; N, 23.82; S, 18.10.
2-Hydroxy-5 pyrimidinesulfonic Acid.

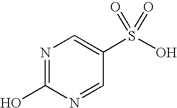

2-Amino-pyrimidine-5-sulfonic acid (0.88 g, 5 mmol), sulfonic acid (5 ml) and H₂O (0.2 ml) was heated at 180° C. for 3 hours. After cooling, the contents of the flask were poured upon 40 g crushed ice. The solid was collected by filtration, washed with water and dried over P₂O₅ in vacuum to afford 0.22 g of a white crystal in 25% yield which was used without further purification.

Anal. Calcd. For C₄H₄N₂O₄S.0.10 H₂O: C, 27.00; H, 2.38; N, 15.74; S. 18.02. Found: C, 26.93; H, 2.37; N, 15.62; S, 18.26.
2-Chloro-5 pyrimidinesulfonyl Chloride.

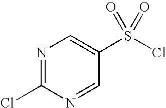

A mixture of phosphorus pentachloride (0.52 g, 2.5 mmol) and 2-hydroxy-5-pyrimidinesulfonic acid was heated in an oil-bath at 180° C. to give a tan-colored liquid, which was refluxed for four hours and then cooled to room temperature. The reaction mixture was then dissolved in ethyl acetate (25 ml). The acetate solution was washed with saturated solution of NaHCO₃, brine, and dried over MgSO₄. The solvent was removed and the product was purified via silica gel chromatography (EtOAc:Hexane=1:2) to provide 0.15 g of a pale white solid in 70% yield.

The title compound was prepared in a manner similar to that used to prepare Example F1 from 1-[4-amino-2-(piperidin-4-ylamino)-thiazol-5-y]-(2,6-difluoro-phenyl)-methanone (Example A6) and 2-chloro-5-pyrimidinesulfonyl chloride to give a white solid in 70% yield.

¹H NMR (DMSO-d₆): δ 9.13 (s, 2H), 8.70 (bs, 1H), 8.02 (bs, 2H), 7.54-7.41 (m, 1H), 7.15 (t, 2H, J=15.9 Hz), 3.58-3.49 (m, 3H), 2.86-2.72 (m, 2H), 2.02-1.85 (m, 2H), 1.63-1.42 (m, 2H). HRMALDIMS: C₁₉H₁₈F₂N₆O₃S₂Cl (MH⁺): 515.0538. Found: 515.0527. Anal. Calcd. For C₁₉H₁₇F₂N₆O₃S₂Cl: C, 44.32; H, 3.33; N, 16.32; S, 12.45. Found: C, 44.18; H, 3.56; N, 16.07; S, 12.16.

### Example F48

### {4-Amino-2-[1-(2-bromo-1-methyl-1H-imidazole-4-sulfonyl)-piperidin-4-ylamino]-thiazol-5-yl}-(2,6-difluoro-phenyl)-methanone

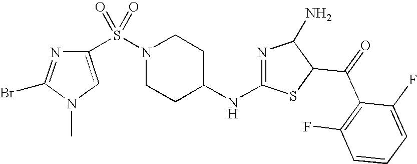

The starting material was prepared as follows:
2-Bromo-1-methyl-1H-imidazole-4-sulfonyl Chloride

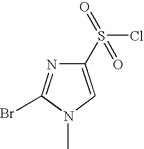

A solution of 1-methyl-1H-imidazole-4-sulfonyl chloride (500 mg, 2.78 mmol) and N-bromosuccinimide (550 mg, 3.06 mmol) in carbon tetrachloride was refluxed for 4 hours. After cooling, the solvent was removed and a solution of the resultant residue in ethyl acetate was washed with brine, dried over MgSO₄, filtered, and concentrated. Column chromatography (60% EtOAc/hexanes) afforded 100 mg of white solid in 14% yield, which was used without any further purification.

¹H NMR (CD₃OD): δ 7.70 (s, 1H), 3.73 (s, 3H).

The title compound was prepared in a manner similar to that used to prepare Example F1 from {4-amino-2-[1-(2-chloro-pyrimidine-5-sulfonyl)-piperidin-4-ylamino]-thiazol-5-yl}-(2,6-difluoro-phenyl)-methanone (Example A6) and 2-bromo-1-methyl-1H-imidazole-4-sulfonyl chloride.

¹H NMR (CD₃OD): δ 7.90 (s, 1H), 7.37 (m, 1H), 7.11-7.02 (m, 2H), 3.80-3.68 (m, 6H), 2.80 (m, 2H), 2.00 (m, 2H), 1.55 (m, 2H). ESIMS (MH⁺): 562. Anal. Calcd for C₁₉H₁₉BrF₂N₆O₃S₂.1.0 Et₂O: C, 43.46; H, 4.60; N, 13.22; S, 10.09. Found: C, 43.72; H, 4.73; N, 13.12; S, 10.01.

### Example F49

### {4-Amino-2-[1-(6-chloro-pyrazine-2-sulfonyl)-piperidin-4-ylamino]-thiazol-5-yl}-(2,6-difluoro-phenyl)-methanone

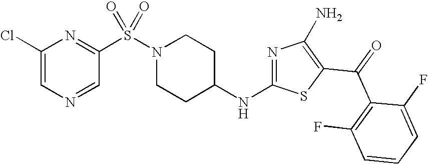

The starting materials were prepared as follows:
6-Chloro-pyrazine-2-sulfonic Acid

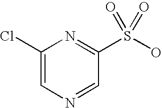

A solution of chloropyrazine (1.7 g, 14.9 mmol) and fuming sulfuric acid (15 ml, 20% free SO₃) was heated at 180° C. for 3 hours. After cooling, the reaction mixture was slowly poured into acetone. The resultant black solid was collected by filtration and rinsed with acetone. The solid was dried over P₂O₅ in vacuum and used without further purification. LC-ESIMS (MH⁺): 194.
6-Chloro-pyrazine-2-sulfonyl Chloride

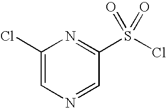

A mixture of 6-chloro-pyrazine-2-sulfonic acid (0.48 g, 2.5 mmol) and phosphorus pentachloride (1.04 g, 5.0 mmol) was heated at 180° C. for 3 hours. The resultant mixture was cooled to room temperature and dissolved in ethyl acetate. The ethyl acetate solution was washed with brine, dried with MgSO₄, filtered and concentrated. Column chromatography afforded 150 mg of white solid in 28% yield, which was used without further purification. LC-ESIMS (MH⁺): 213.

The title compound was prepared in a manner similar to that used to prepare Example F1 from [4-Amino-2-(piperidin4-ylamino)-thiazol-5-yl]-(2,6-difluoro-phenyl)-methanone (Example A6) and 6-chloro-pyrazine-2-sulfonyl chloride in 15% yield.

¹H NMR (CD₃OD): δ 8.92 (d, 1H, J=1.51 Hz), 8.83 (d, 1H, J=1.51 Hz), 7.44 (m, 1H), 7.07-6.96 (m, 2H), 3.87-3.76 (m, 3H), 3.00 (m, 2H), 1.96 (m, 2H), 1.48 (m, 2H). TOFMSES⁺. Calcd for C₁₉H₁₇ClF₂N₆O₃S₂ (MH⁺): 515.0538. Found: 515.0530

### Example F50

### 1-{4-Amino-2-[1-(5-bromo-thiophene-2-sulfonyl)-piperidin4-ylamino]-thiazol-5-yl}1-(2,6-difluoro-phenyl)-methanone

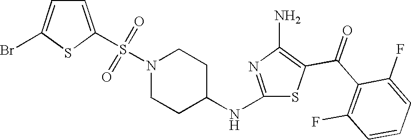

The title compound was prepared in a manner similar to that used to prepare Example F1 from [4-Amino-2-(piperidin-4-ylamino)-thiazol-5-yl]-(2,6-difluoro-phenyl)-methanone (Example A6) and 5-bromo-thiophene-2-sulfonyl chloride.

¹H NMR (DMSO d₆): δ 8.80 (bs,1H), 8.03 (bs, 1H), 7.47-7.42 (m, 2H), 7.16-7.11 (m, 2H) 3.45-3.41 (m, 2H), 2.66 (m, 2H), 1.97-1.89 (m, 2H), 1.54-1.48 (m, 2H). Anal. Calcd for C₁₉H₁₇F₂N₄O₃S₃.0.1 Et₂O: C, 40.78; H, 2.99; N, 9.80. Found: 41.01; H, 3.18; N, 9.75.

### Example F51

### {4-Amino-2-[1-(thiophene-2-sulfonyl)-piperidin4-ylamino]-thiazol-5-yl}-(2,6-difluoro-phenyl)-methanone

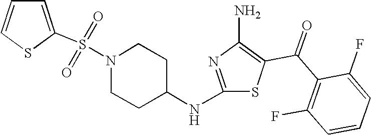

The title compound was prepared in a manner similar to that used to prepare Example F1 from [4-Amino-2-(piperidin4-ylamino)-thiazol-5-yl]-(2,6-difluoro-phenyl)-methanone (Example A6) and thiophene-2-sulfonyl chloride.

¹H NMR (CD₃OD): δ 7.87 (dd, J=1.1, 5.1 Hz, 1H), 7.61 (dd, J=1.1, 5.1Hz, 1H), 7.46 (m, 1H), 7.25(m, 1H), 7.03 (m, 2H), 3.66 (m, 3H), 2.65 (m, 2H), 2.10 (m, 2H), 1.65(m, 2H). Anal. Calcd for C₁₉H₁₇F₂N₄O₃S₃.0.2 Et₂0.0.35 H₂O: C, 40.78; H, 2.99; N, 9.80. Found: 46.98; H, 4.09; N, 11.07.

### Example F52

### (4-Amino-2-{1-[4-(1-methyl-pyrrolidin-3R-yl)-benzenesulfonyl]-piperidin-4-ylamino}-thiazol-5-yl)-(2,6-difluoro-phenyl)-methanone

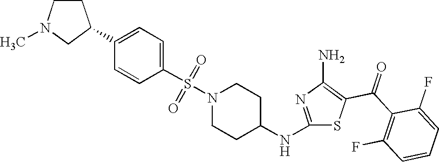

The starting materials were prepared as follows:
1-Methyl-3R-phenyl-pyrrolidine

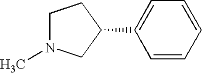

To a solution of 3R-phenylpyrrolidine (0.51 g, 3.46 mmol; Chung, et al, J. Org. Chem., 55, 270-275 (1990)) in formic acid (1 ml) was added 37% aqueous formaldehyde (2 ml). The resultant solution was refluxed for 1.5 hours and diluted with H₂O (20 ml). The aqueous layer was brought to pH 9 with 2N NaOH and extracted with CHCl₃ (50 ml×2). The combined organic layers were dried over Na₂SO₄, filtered, and concentrated in vacuo to afford 0.557 g of brown oil in 100% yield and used without further purification.

¹H NMR matched that of 1-methyl-3-phenyl-pyrrolidine of Example F25.

The title compound was prepared in manner analogous to that used for preparation of 1-(4-amino-2-[1-[4-(1-methyl-pyrrolidin-3-yl)-benzenesulfonyl]-piperidin-4-ylamino]-thiazol-5-yl)-1-(2,6-difluoro-phenyl)-methanone in Example F25 and azeotroped with n-heptane to provide 0.46 g (69%) of yellow foam. Purified by chiral HPLC with a Chiralpak AS 4.6×250 mm column at 40° C. and eluted with 0.1% diethylamine in EtOH:hexanes (40:60) at 0.5 mL/min, retention time 16.3 min.

¹HNMR (CD₃OD): δ 7.70 (d, 2H, J=8.4 Hz), 7.52 (d, 2H, J=8.4 Hz), 7.44-7.36 (m, 1H), 7.00 (dd, 2H, J=7.5, 8.3 Hz), 3.52 (dd, 1H, J=7.8, 9.1 Hz), 3.08 (dd, 1H, J=8.4, 9.4 Hz), 2.44 (s, 3H). LC-ESIMS (MH⁺): 562.10 Anal. Calcd for C₂₆H₂₉F₂N₅O₃S₂.0.1CH₃CN.1.3H₂.0.3 heptane: C, 54.89; H, 5.97; N, 11.54; S, 10.36. Found: C, 55.37; H, 5.94; N, 11.88; S, 9.98.

### Example F53

### (4-Amino-2-{1-[4-(1-methyl-pyrrolidin-3S-yl)-benzenesulfonyl]-piperidin-4-ylamino}-thiazol-5-yl)-(2,6-difluoro-phenyl)-methanone

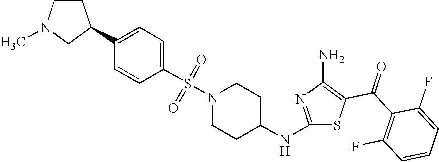

The title compound was prepared in a manner analogous to that used for Example F47, originating from (−)-3S-phenylpyrrolidine (Chung, et al, J. Org. Chem., 55, 270-275 (1990)) to provide 0.38 g of yellow foam in 57% yield from 1-methyl-3S-phenylpyrrolidine. Purified by chiral HPLC with a Chiralpak AS 4.6×250 mm column at 40° C. and eluted with 0.1% diethylamine in EtOH:hexanes (40:60) at 0.5 mL/min, retention time 11.8 min.

¹HNMR and MS identical to Example F47. Anal. Calcd for C₂₆H₂₉F₂N₅O₃S₂.1.0 H₂.0.2 heptane: C, 54.87; H, 5.75; N, 11.68; S, 10.69. Found: C, 54.80; H, 5.76; N, 11.83; S, 10.32.

### Example F54

### [4-Amino-2-(1-ethenesulfonyl-piperidin-4-ylamino)-thiazol-5-yl]-(2,6-difluoro-phenyl)-methanone

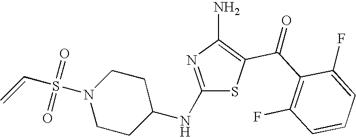

To [4-amino-2-(piperidin-4-ylamino)-thiazol-5-yl]-(2,6-difluoro-phenyl)-methanone (2.00 g, 5.92 mmol; Example A6) and triethylamine (1.65 ml, 11.8 mmol) in anhydrous THF (100 ml) stirred at 0° C., was added dropwise a solution of ethenesulfonyl chloride (0.969 9, 7.70 mmol, see Rondestvedt, et al., J. Amer. Chem. Soc., 76, 1926-1929 (1954)) in THF (20 ml). The yellow suspension stirred at 0° C. for 10 min, acidified to pH 3 with 1N HCl, and the solvent removed. The resultant residue was dissolved in MeOH (5 ml), cooled with ice-water bath, and diluted with 1N HCl (100 ml). After stirring rapidly for 20 min., a white solid was filtered off, washed with water, and dried under vacuum. Column chromatography with 2.5% MeOH in CHCl₃ provided 2.15 g of white solid in 85% yield, which was used without any further purification.

¹H NMR (DMSO-d₆): δ 8.84 (bs, 1H), 8.07 (bs, 2H), 7.50 (m, 1H), 7.17 (dd, 2H, J=7.7, 8.0 Hz), 6.79 (dd, 1H, J=10.1, 16.6 Hz), 6.14 (d, 1H, J=10.1 Hz), 6.10 (d, 1H, J=16.6 Hz), 3.05 (m, 1H), 2.79 (t, 2H, J=10.6 Hz). ESMS (M+H+): 429.

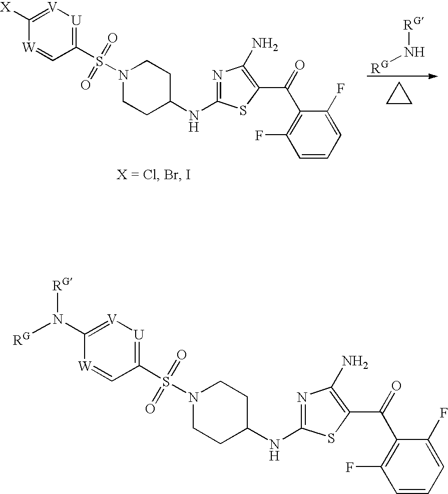

### Example G1

### 1-[4-Amino-2-{1-[6-(2-dimethylamino-ethyl)-amino-pyridine-3-sulfonyl]-piperidin-4-ylamino)thiazol-5-yl]-1-(2,6-difluoro-phenyl)-methanone Trifluoroacetic Acid Salt

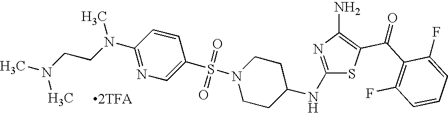

The title compound was prepared as follows. A suspension of 1-{4-amino-2-[1-(6-chloro-pyridine-3-sulfonyl)-piperidin-4-ylamino]-thiazol-5-yl}-1-(2,6-difluoro-phenyl)-methane (Example F21; 154 mg, 0.300 mmol) and N,N,N′-trimethyl-ethane-1,2-diamine (61 mg, 6.0 mmol) in ethylene glycol (5 ml) was heated in a microwave oven (0.7 cu. Ft., 800 watt) for two 30 second intervals. The resultant solution was allowed to cool, diluted with ethyl acetate, washed with aqueous NaHCO₃, and concentrated to give a solid, which was purified via preparative HPLC to obtain a 67% yield.

¹H NMR (CD₃OD): δ 8.51 (d, 1H, J=2.2 Hz), 7.91 (dd, 1H, J=2.2, 9.1Hz), 7.51-7.36 (m, 1H), 7.03 (m, 2H), 6.84 (d, 1H, J=9.1 Hz), 4.09 (t, 2H, J=6.0 Hz), 3.64 (m, 3H), 3.45 (t, 2H, J=6.0 Hz), 3.18 (s, 3H), 3.02 (s, 6H), 2.50 (m, 2H), 2.10 (m, 2H), 1.72 (m, 2H). HRMALDIMS. Calcd. For C₂₅H₃₁F₂N₇O₃S₂Na (MNa⁺): 602.1790. Found: 602.1777. Anal. Calcd. For C₂₅H₃₁F₂N₇O₃S₂.1.95 TFA: C, 43.28; H, 4.14; N, 12.23; S, 8.00. Found: C, 43.39; H, 4.12; N, 12.14; S, 8.02.

The compounds of the following Examples from G2 to G17, and G19 to G21 were prepared in a manner similar to that for Example G1, from 1-{4-amino-2-[1-(6-chloro-pyridine-3-sulfonyl)-piperidin-4-ylamino]-thiazol-5-yl}-1-(2,6-difluoro-phenyl)-methanone (Example F21) and corresponding amines.

### Example G2

### 1-(4-Amino-2{1-[6-(2-dimethylamino-ethylamino)-pyridine-3-sulfonyl]-piperidin-4-ylamino}-thiazol-5-yl)-1-(2,6-difluoro-phenyl)-methanone

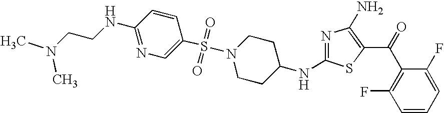

¹H NMR (DMSO-d₆): δ 7.53 (d, 1H, J=2.45 Hz), 7.85 (dd, 1H, J=2.5, 9.0 Hz), 6.67-6.53 (m, 1H), 6.24-6.12 (m, 2H), 7.78 d, (1H, J=9.0 Hz), 2.83-2.69 (m, 5H), 1.87-1.71 (m, 4H), 1.32-1.18 (m, 2H), 0.89-0.72 (m, 2H). HRMALDIMS. Calcd for C₂₄H₃₀F₂N₇O₃S₂ (MH⁺): 566.1814. Found: 566.1832

### Example G3

### 1-(4-Amino-2{1-[6-(2-hydroxy-ethylamino)-pyridine-3-sulfonyl]-piperidin-4-ylamino}-thiazol-5-yl)-1-(2,6-difluoro-phenyl)-methanone

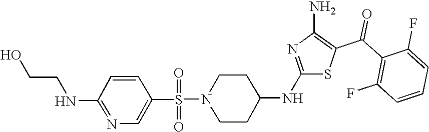

¹H NMR (DMSO-d₆): δ 7.50 (d, 1H, J=2.3 Hz), 6.84 (dd, 1H, J=2.6, 8.9 Hz), 6.68-6.54 (m, 1H), 6.24-6.13 (m, 2H), 5.81 (d, 1H, J=9.1 Hz), 2.93-2.88 (m, 2H), 2.87-2.60 (m, 5H), 1.83-1.72 (m, 2H), 0,89-0.73 (m, 2H). HRMALDIMS. Calcd for C₂₂H₂₅F₂N₆O₄S₂ (MH⁺): 539.1341. Found: 539.1335

### Example G4

### 1-(4-Amino-2-{1-[6-(1-oxo-thiomorpholine-4-yl)-pyridine-3-sulfonyl]-piperidin-4-ylamino}-thiazol-5-yl)-1-(2,6-difluoro-phenyl)-methanone

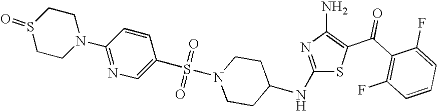

¹H NMR (Acetone-d₆): δ 8.46 (d, 1H, J=2.5 Hz), 7.82 (d, 1H, J=2.6, 9.0 Hz), 7.53-7.42 (m, 1H), 7.12-7.00 (m, 3H), 4.46-4.34 (m, 2H), 4.20-4.07 (m, 2H), 3.68-3.52 (m, 3H), 3.07-2.83 (m, 4H), 2.80-2.70 (m, 2H), 2.67-2.58 (m, 2H), 1.78-1.60 (m, 2H). HRMALDIMS. Calcd for C₂₄H₂₇F₂N₆O₄S₃(MH⁺) 597.1218. Found: 597.1220

### Example G5

### 1-(4-Amino-2-{1-[6-(4-methyl-piperazin-1-yl)-pyridine-3-sulfonyl]-piperidin-4-ylamino}-thiazol-5-yl)-1-(2,6-difluoro-phenyl)-methanone

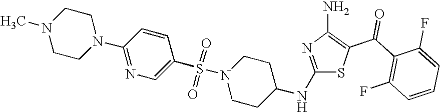

¹H NMR (CD₃OD): δ 8.46 (d, 1H, J=2.1. Hz), 7.84 (dd, 1H, J=2.1, 8.0Hz), 7.45 (m, 1H), 7.04 (m, 2H), 6.92 (d, 1H, J=8.0 Hz), 3.78 (m, 4H), 3.60 (m, 3H), 2.54 (m, 6H), 2.38 (s, 3H), 2.08 (m, 2H), 1.62 (m, 2H). Anal. Calcd for C₂₅H₂₉F₂N₇O₃S₂.0.9 Et₂O: C, 53.31; H, 5.94; N, 15.22; S, 9.95. Found: C, 53.08; H, 5.93; N, 14.93; S, 9.74.

### Example G6

### 1-{4-Amino-2-[1-(6-piperazin-1-yl-pyridine-3-sulfonyl)-piperidin-4-ylamino]-thiazol-5-yl}-1-(2,6-difluoro-phenyl)-methanone

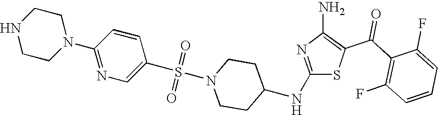

¹H NMR (CD₃OD): δ 8.46 (d, 1H, J=2.0 Hz), 7.80 (dd, 1H, J=2.0, 8.1 Hz), 7.44 (m, 1H), 7.02 (m, 2H), 6.88 (d, 1H, J=8.1 Hz), 3.74 (m, 4H), 3.62 (m, 3H), 2.95 (m, 4H), 2.60 (m, 2H), 2.10 (m, 2H), 1.64 (m, 2H). HRMALDIMS. Calcd for C₂₄H₂₈F₂N₇O₃S₂ (MH⁺): 564.1618. Found: 564.1627

### Example G7

### 1-{4-Amino-2-[1-(6-methylamino-pyridine-3-sulfonyl)-piperidin-4-ylamino]-thiazol-5-yl}-1-(2,6-difluoro-phenyl)-methanone

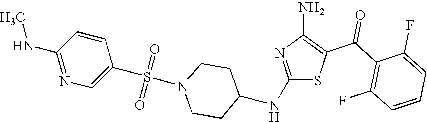

¹H NMR (CD₃OD): δ 8.28 (d, 1H, J=2.5 Hz), 7.92 (dd, 1H, J=2.5, 8.1 Hz), 7.46 (m, 1H), 7.04 (m, 2H), 6.92 (d, 1H, J=8.1 Hz), 3.70 (m, 3H), 3.06 (s, 3H), 2.72 (m, 2H), 2.12 (m, 2H), 1.66 (m, 2H). HRMALDIMS. Calcd for C₂₁H₂₂F₂N₆O₃S₂ (MH⁺): 509.1236. Found: 509.1229.

### Example G8

### 1-{4-Amino-2-[1-(6-amino-pyridine-3-sulfonyl)-piperidin-4-ylamino]-thiazol-5-yl}-1-(2,6-difluoro-phenyl)-methanone

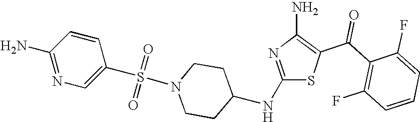

¹H NMR (CD₃OD): δ 8.36 (d, 1H, J=1.8 Hz), 8.04 (dd, 1H, J=1.8, 8.1 Hz), 7.80 (m, 1H), 7.04 (m, 3H), 3.72 (m, 3H), 2.78 (m, 2H), 2.16 (m, 2H), 1.70 (m, 2H). HRMALDIMS. Calcd for C₂₀H₂₁F₂N₆O₃S₂ (MH⁺): 495.1079. Found: 495.1076.

### Example G9

### 1-{4-Amino-2-[1-(4Hydroxy-3,4,5,6-tetrahydro-2H-[1,2′]bipyridinyl-5′-sulfonyl)-piperidin-4-ylamino]-thiazol-5-yl}-1-(2,6-difluoro-phenyl)-methanone

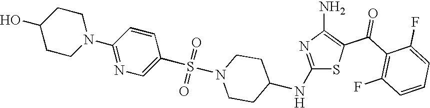

¹H NMR (CD₃OD): δ 8.40 (d, 1H, J=2.0 Hz), 7.82 (dd, 1H, J=2.0, 8.2 Hz), 7.46 (m, 1H), 7.06 (m, 3H), 4.18 (m, 2H), 3.94 (m, 1H), 3.80-3.60 (m, 3H), 3.40 (m, 2H), 2.62 (m, 2H), 2.10 (m, 2H), 1.98 (m, 2H), 1.70-1.50 (m, 4H). HRMALDIMS. Calcd for C₂₅H₂₉F₂N₆O₄S₂ (MH⁺): 579.1654. Found: 579.1653.

### Example G10

### 1-(4-Amino-2-{1-6-[(2-hydroxy-ethyl)-methyl-amino]-pyridine-3-sulfonyl}-piperidin-4-ylamino)-thiazol-5-yl]-1-(2,6-difluoro-phenyl)-methanone Trifluoroacetic Acid Salt

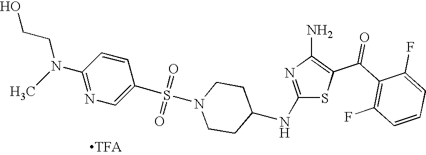

Purified via preparative HPLC.

¹H NMR (DMSO-d₆): δ 8.80 (br, 1H), 8.33 (d, 1H, J=2.2 Hz), 8.03 (bs, 2H), 7.74-7.65 (dd, 1H, J=2.2, 9.2 Hz), 7.54 (m, 1H), 7.18 (m, 2H), 6.78 (d, 1H, J=9.2 Hz), 3.70-3.52 (m, 5H), 3.48 (m, 2H), 3.13 (s, 3H), 2.65 (m, 2H), 1.98 (m, 2H), 1.63 (m, 2H). HRMALDIMS. Calcd. For C₂₃H₂₆F₂N₆O₄S₂Na (MNa⁺): 575.1317. Found: 575.1308. Anal. Calcd. For C₂₃H₂₆F₂N₆O₄S₂.1.28 TFA: C, 43.94; H, 3.94; N, 12.03; S, 9.18. Found: C, 44.02; H, 3.91; N, 11.89; S, 9.01.

### Example G11

### 1-(4-Amino-2-{1-[6-(3-hydroxy-pyrrolidin-1-yl)-pyridin-3-sulfonyl]-piperidin-4-ylamino}-thiazol-5-yl)-1-(2,6-difluoro-phenyl)-methanone Trifluoroacetic Acid Salt

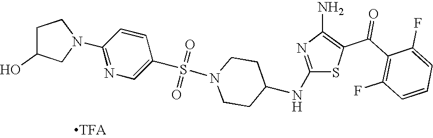

Purified via preparative HPLC.

¹H NMR (DMSO-d₆): δ 8.80 (br, 1H), 8.35 (d, 1H, J=2.2 Hz), 8.02 (bs, 2H), 7.76-7.68 (dd, 1H, J=2.2, 9.0 Hz), 7.54-7.42 (m, 1H), 7.2 (m, 2H), 6.69 (d, 1H, J=9.0 Hz), 4.48-4.35 (m, 3H), 3.67-3.35 (m, 7H), 2.13-1.82 (m, 4H), 1.63 (m, 2H). HRMALDIMS. Calcd. For C₂₄H₂₇F₂N₆O₄S₂ (MH⁺): 565.1498. Found: 565.1493. Anal. Calcd. For C₂₄H₂₆F₂N₆O₄S₂.1.30 TFA: C, 44.82; H, 3.86; N, 11.79; S, 9.00. Found: C, 44.87; H, 3.94; N, 11.80; S, 8.94.

### Example G12

### 1-{4-Amino-2-[1-(3-hydroxy-3,4,5,6-tetrahydro-2H-[1,2′]bipyridinyl-5′-sulfonyl)-piperidin-4-ylamino]-thiazol-5-yl}-1-(2,6-difluoro-phenyl)-methanone Trifluoroacetic Acid Salt

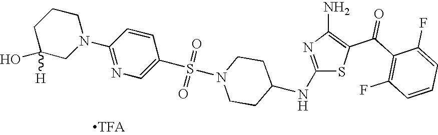

Purified via preparative HPLC.

¹H NMR (DMSO-d₆): δ 8.84 (br, 1H), 8.39 (d, 1H, J=2.2 Hz), 8.05 (bs, 2H), 7.74 (dd, 1H, J=2.2, 9.1 Hz), 7.62-7.44 (m, 1H), 7.19 (m, 2H), 6.94 (d, 1H, J=9.1 Hz), 4.19 (m, 3H), 3.90 (m, 1H), 3.62-3.33 (m, 4H), 3.28 (m, 1H), 3.05 (m, 1H), 2.04-1.89 (m, 4H), 1.83 (m, 1H), 1.68 (m, 5H). HRMALDIMS. Calcd. for C₂₅H₂₉F₂N₆O₄S₂ (MH⁺): 601.1474. Found: 601.1459. Anal. Calcd. For C₂₅H₂₈F₂N₆O₄S₂.1.26 TFA: C, 45.76; H, 4.08; N, 11.64; S, 8.88. Found: C, 45.73; H, 4.17; N, 11.73; S, 8.65.

### Example G13

### 1-{4-Amino-2-{1-[6-(2R-hydroxymethyl-pyrrolidin-1-yl)-pyridine-3-sulfonyl]-piperidin-4-ylamino}-thiazol-5-yl)-1-(2,6-difluoro-phenyl)-methanone Trifluoroacetic Acid Salt

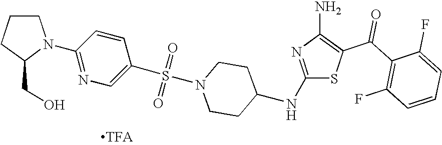

Purified via preparative HPLC.

¹H NMR (DMSO-d₆): δ 8.80 (br, 1H), 8.32 (d, 1H, J=2.2 Hz), 8.01 (bs, 2H), 7.75-7.68 (dd, 1H, J=2.2, 8.5 Hz), 7.58 (m, 1H), 7.14 (m, 2H), 6.64 (d, 1H, J=8.5 Hz), 4.21-4.06 (m, 2H), 3.59-3.30 (m, 7H), 2.11-1.85 (m, 7H), 1.63 (m, 2H). ESIMS (MH⁺): 579. Anal. Calcd. For C₂₅H₂₈F₂N₆O₄S₂.1.48 TFA: C, 44.93; H, 3.98; N, 11.24; S, 8.58. Found: C, 44.91; H, 3.95; N, 11.16; S, 8.68.

### Example G14

### 1-{4-Amino-2-{1-[6-(2S-hydroxymethyl-pyrrolidin-1-yl)-pyridine-3-sulfonyl]-piperidin-4-ylamino}-thiazol-5-yl)-1-(2,6-difluoro-phenyl)-methanone Trifluoroacetic Acid Salt.

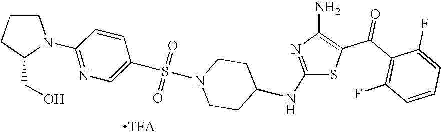

Purified via preparative HPLC.

¹H NMR (DMSO-d₆): δ 8.80 (br, 1H), 8.32 (d, 1H, J=2.2 Hz), 8.01 (bs, 2H), 7.75-7.68 (dd, 1H, J=2.2, 8.5 Hz), 7.58 (m, 1H), 7.14 (m, 2H), 6.64 (d, 1H, J=8.5 Hz), 4.21-4.06 (m, 2H), 3.59-3.30 (m, 7H), 2.11-1.85 (m, 7H), 1.63 (m, 2H). ESIMS (MH⁺): 579. Anal. Calcd. For C₂₅H₂₈F₂N₆O₄S₂.1.53 TFA: C, 44.75; H, 3.95; N, 11.16; S, 8.52. Found: C, 44.67; H, 4.01; N, 11.23; S, 8.68.

### Example G15

### 1-(4-Amino-2-{1-[6-(3,5-dimethyl-piperizin-1-yl)-pyridin-3-sulfonyl]-piperidin-4-ylamino}-thiazol-5-yl)-1-(2,6-difluoro-phenyl)-methanone Trifluoroacetic Acid Salt.

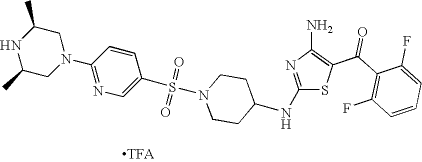

Purified via preparative HPLC.

¹H NMR (DMSO-d₆): δ 8.42 (d, 1H, J=2.2 Hz), 8.02 (bs, 2H), 7.84 (dd, 1H, J=2.3, 9.0 Hz), 7.56 (m, 1H), 7.21-7.10 (m, 3H), 4.71-4.62 (m, 4H), 3.52-3.26 (m, 5H), 2.93 (m, 2H), 2.76 (s, 1H), 2.01 (m, 2H), 1.61 (m, 2H), 1.29 (d, 6H, J=6.5 Hz). ESIMS (MH⁺): 592. Anal. Calcd. For C₂₆H₃₁F₂N₇O₃S₂.1.30 H₂O.1.53 TFA: C, 42.22; H, 4.21; N, 11.47; S, 7.50. Found: C, 42.43; H, 4.18; N, 11.34; S, 7.25.

### Example G16

### 4-({5-[4-Amino-5-(2,6-difluoro-benzoyl)-thiazol-2-ylamino]-piperidine-1-sulfonyl}-pyridin-2-yl)-piperazine-1-carboxaldehyde Trifluoroacetic Acid Salt.

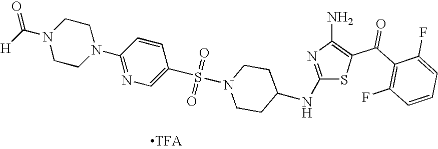

Purified via preparative HPLC.

¹H NMR (CD₃OD): δ 8.35 (d, 1H, J=2.2 Hz), 8.03 (s, 1H), 7.78-7.70 (dd, 1H, J=2.2, 9.0 Hz), 7.33 (m, 1H), 6.94-6.82 (m, 3H), 3.85 (m, 1H), 3.78-3.64 (m, 4H), 3.58-3.42 (m, 7H), 2.57 (m, 2H), 2.03 (m, 2H), 1.71 (m, 2H). HRMALDIMS. Calcd. For C₂₅H₂₈F₂N₇O₄S₂ (MH⁺): 592.1607. Found: 592.1605. Anal. Calcd. For C₂₅H₂₇F₂N₇O₄S₂.0.28 H₂O.2.03 TFA: C, 42.14; H, 3.60; N, 11.84; S, 7.74. Found: C, 42.13; H, 3.75; N, 11.83; S, 7.67.

### Example G17

### 1-[4-Amino-2-(1-{6-[((R)-2-hydroxy-propyl)-methyl-amino]-pyridine-3-sulfonyl}-piperidine-4-ylamino)-thiazol-5-yl]-1-(2,6-difluoro-phenyl)-methanone Trifluoroacetic Acid Salt.

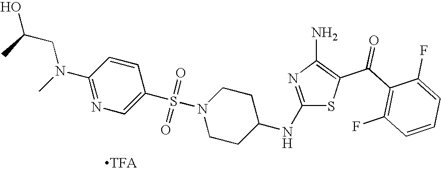

Purified via preparative HPLC.

¹H NMR (CD₃OD): δ 8.38 (d, 1H, J=2.4 Hz), 7.86 (dd, 1H, J=2.4, 9.0 Hz), 7.44 (m, 1H), 7.08-6.92 (m, 2H; d, 1H, J=9.0 Hz), 4.18 (m, 1H), 3.74-3.65 (m, 5H), 3.24 (s, 3H), 2.68 (m, 2H), 2.18 (m, 2H), 1.78 (m, 2H), 1.24 (d, 3H, J=6.3 Hz). HRMALDIMS. C₂₄H₂₈F₂N₆O₄S₂Na (MNa⁺): 589.1474. Found: 589.1453. Anal. Calcd. For C₂₄H₂₈F₂N₆O₄S₂.1.89 TFA: C, 42.66; H, 3.85; N, 10.75; S, 8.20. Found: C, 42.62; H, 3.98; N, 10.79; S, 8.20.

### Example G18

### 1-(4-Amino-2-{1-[6-((S)-1-methyl-piperidin-3-ylmethoxy)-pyridine-3-sulfonyl]-piperidin-4-ylamino}-thiazol-5-yl)-1-(2,6-difluoro-phenyl)-methanone.

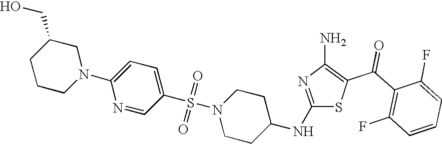

Obtained as a minor impurity from the preparation of Example H11. Isolated after radial chromatography and recrystallized from MeOH to give 30 mg of a colorless amorphous solid in 8% yield, mp>149° C. (d).

¹H NMR (CD₃OD): δ 8.40 (d, 1H, J=2.5 Hz), 7.91 (s, 1H), 7.75 (dd, 1H, J=2.5, 9.2 Hz), 7.44 (ddd, 1H, J=6.5, 8.3, 14.9 Hz), 7.02 (ddd, 2H, J=3.3, 8.3, 15.8 Hz), 6.88 (d, 1H, J=9.2 Hz), 4.45 (d, 1H, J=13.3 Hz), 4.43 (d, 1H, J=14.0 Hz), 3.10 (ddd, 1H, J=3.1, 10.1, 13.7 Hz), 2.90 (dd, 1H, J=10.3, 13.2 Hz), 2.61 (t, 2H, J=10.9 Hz), 2.09 (d, 2H, J=13.0 Hz). FTIR (KBr): 3402, 3294, 3220, 1618, 1590, 1547, 1506, 1464, 1373, 1309, 1170, 1141, 1106, 1002 cm⁻¹. LC-ESIMS: (MH⁺) 593.15 Anal. Calcd. for C₂₆H30F₂N₆O₄S₂.1.5 H₂O: C, 50.39; H, 5.37; N, 13.56; S, 10.35. Found: C, 50.42; H, 5.29; N, 13.48; S, 10.30.

### Example G19

### 1-(4-Amino-2-{1-[6-(2,3-dihydroxy-propylamino)-pyridine-3-sulfonyl]-piperidin-4-ylamino}-thiazol-5-yl)-1-(2,6-difluoro-phenyl)-methanone.

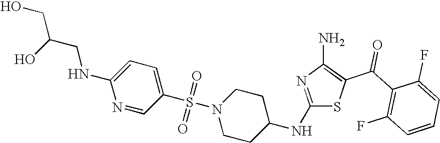

¹H NMR (CD₃OD): δ 8.31 (d, 1H, J=2.4 Hz), 7.82 (dd, 1H, J=2.4, 8.8 Hz), 7.49 (m, 1H), 7.04 (m, 2H), 6.88 (d, 1H, J=8.8 Hz), 3.86 (m, 1H), 2.70-3.44 (m, 7H), 2.68 (m, 2H), 2.10 (m, 2H), 1.66 (m, 2H). HRMALDIMS: Calcd. For C₂₃H₂₇F₂N₆O₅S₂ (MH⁺): 569.1447. Found: 569.1432.

### Example G20

### 1-(4-Amino-2-{1-[6-(2-methylamino-ethylamino)-pyridine-3-sulfonyl]-piperidin-4-ylamino}-thiazol-5-yl)-1-(2,6-difluoro-phenyl)-methanone.

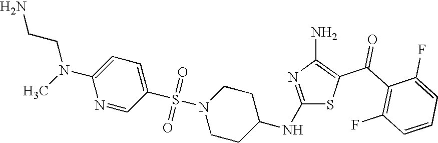

¹H NMR (CD₃OD): δ 8.49 (s, 1H), 7.75 (m, 1H), 7.44 (m, 1H), 7.03 (t, 2H, J=8.4 Hz), 6.82 (d, 1H, J=9.1 Hz), 3.98 (t, 2H, J=5.9 Hz), 3.69-3.58 (m, 3H), 3.25 (t, 2H, J=5.8 Hz), 3.18 (s, 3H), 2.58 (m, 2H), 2.12 (m, 2H), 1.65 (m, 2H). HRFABMS: Calcd. for C₂₃H₂₈F₂N₈O₂S₂Na (MNa⁺): 574.1477. Found: 574.1501.

### Example G21

### 1-(4-Amino-2-{1-[6-(4,4-dimethyl-4,5-dihydro-imidazol-1-yl)-pyridine-3-sulfonyl]-piperidin-4-ylamino}-thiazol-5-yl)-1-(2,6-difluoro-phenyl)-methanone.

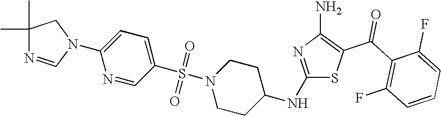

¹H NMR (DMSO-d₆): δ 8.86 (br, 1H), 8.56 (s, 1H), 8.10 (s, 1H), 8.04 (m, 3H), 7.54 (m, 1H), 7.18 (m, 3H), 3.64 (s, 2H), 3.50 (m, 2H), 2.66 (m, 2H), 2.00 (m, 2H), 1.60 (m, 2H), 1.34 (s, 6H). Anal. Calcd. for C₂₅H₂₇F₂N₇O₃S₂.0.3 EtOAc: C, 52.26; H, 4.92; N, 16.29; S, 10.65. Found; C, 52.07; H, 4.89; N, 16.34; S, 10.71.

### Example G22

### 1-(4-Amino-2-{1-[6-(3,3-dimethyl-piperazin-1-yl)-pyridine-3-sulfonyl]-piperidin-4-ylamino}-thiazol-5-yl)-1-(2,6-difluoro-phenyl)-methanone.

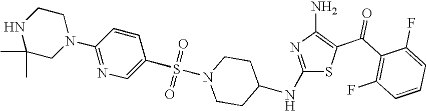

2,2-Dimethylpiperazine (89 mg, 0.78 mmol; Bogeso, et al., J. Med. Chem., 38, 4380-4392 (1995)) and Et₃N (0.108 ml, 0.778 mmol) were added to a suspension of 1-{4-amino-2-[1-(6-chloro-pyridine-3-sulfonyl)-piperidin-4-ylamino]-thiazol-5-yl}-1-phenyl-methanone (Example F21; 200 mg, 0.289 mmol) in acetonitrile (1 ml). The mixture was heated at 85° C. for 3 hours and allowed to cool to ambient temperature. Precipitation and rinse with 2% MeOH/ether and subsequent drying provided 120 mg of a white solid in 50% yield.

¹H NMR (CD₃OD): δ 8.40 (s, 1H), 7.82 (dd, 2H, J=2.5, 9.1 Hz), 7.48-7.38 (m, 1H), 7.0 (dd, 2H, J=7.4, 8.4 Hz), 6.88 (d, 1H, J=9.3 Hz), 2.96 (bs, 2H), 2.58 (dd, 2H, J=10.5, 10.6 Hz), 1.14(s,6H). Anal. Calcd. for C₂₆H₃₁F₂N₇O₃S₂.0.3 H₂O: C, 52.30; H, 5.33; N, 16.42; S, 10.74. Found; C, 51.97; H, 5.23; N, 16.30; S, 10.67.

### Example G23

### 1-(4-Amino-2-{1-[6-(2,4-dimethyl-4,5-dihydro-imidazol-1-yl)-pyridin-3-sulfonyl]-piperidin-4-ylamino}-thiazol-5-yl)-1-(2,6-difluoro-phenyl)-methanone Hydrochloride.

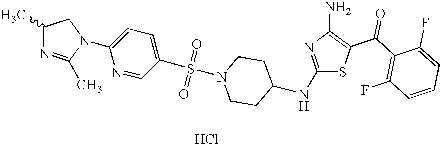

The title compound was prepared as follows. 1-{4-Amino-2-[1-(6-chloro-pyridine-3-sulfonyl)-piperidin-4-ylamino]-thiazol-5-yl}-1-(2,6-difluoro-phenyl)-methanone (Example F21; 100 mg, 0.200 mmol) and 2,4-dimethyl-imidazoline (100 mg, 1.00 mmol) in DMSO (2 ml) were heated in a microwave oven (0.7 cu. Ft., 800 watt) for two 45 second intervals. The resultant solution was allowed to cool, diluted with ethyl acetate, washed with sat. NaHCO₃ and brine, dried over MgSO₄, filtered, and concentrated in vacuo. Purification via preparative HPLC and treatment of the fractions with aqueous HCl prior to lyophilization afforded 48 mg of yellow solid in 84% yield. ¹H NMR (DMSO-d₆): δ 8.78 (br, 1H), 8.52 (s, 1H), 8.06-7.91 (m, 3H), 7.50 (m, 1H), 7.14 (m, 2H), 6.99 (d, 1H, J=9.1 Hz), 4.04 (m, 2H), 3.52-3.38 (m, 3H), 2.68-2.57 (m, 3H), 2.41 (s, 3H), 1.94 (m, 2H), 1.52 (m, 2,H), 1.21 (d, 3H, J=5.7 Hz). HRFABMS. Calcd.for C₂₅H₂₈F₂N₇O₃S₂ (MH⁺): 576.1658. Found: 576.1677. Anal. Calcd. For C₂₅H₂₇F₂N₇O₃S₂.0.80 HCl: C, 50.99; H, 4.76%, N, 16.65; S, 10.89. Found: C, 50.96; H, 4.93; N, 16.56; S, 10.89.

### Example G24

### 1-[4-Amino-2-(1-{5-bromo-6-[(2-dimethylamino-ethyl)-methyl-amino]-pyridine-3-sulfonyl}-piperidin-4-ylamino)-thiazol-5-yl]-1-(2,6-difluoro-phenyl)-methanone.

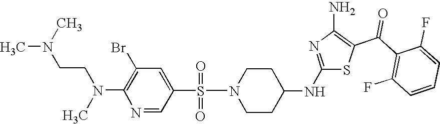

Prepared in a manner similar to that for Example G1. 1-{4-Amino-2-[1-(5-bromo-6-chloro-pyridine-3-sulfonyl)-piperidin-4-ylamino]-thiazol-5-yl}-1-(2,6-difluoro-phenyl)-methanone (Example F16) and N, N, N′-trimethyl-ethane-1,2-diamine gave 96 mg of white solid in 68% yield.

¹H NMR (DMSO-d₆): δ 8.80 (br, 1H), 8.39 (s, 1H), 8.00 (br, 3H), 7.48 (m, 1H), 7.14 (t, 2H, J=7.7 Hz), 3.65 (t, 2H, J=6.6 Hz), 3.51-3.40 (m, 2H), 3.35-3.27 (m, 2H), 3.13 (s, 3H), 2.17 (s, 6H), 2.02-1.87 (m, 2H), 1.60-1.44 (m, 2H). ESIMS (MH⁺): 658/656. Anal. Calcd. for C₂₅H₃₀BrF₂N₇O₂S₂.0.8 H₂O: C, 44.61; H, 4.73; N, 14.57; S, 9.53. Found: C, 44.53; H, 4.83; N, 14.46; S, 9.72.

### Example G25

### 1-{4-Amino-2-[1-(6-imidazol-1-yl-pyridine-3-sulfonyl)-piperidin-4-ylamino]-thiazol-5-yl}-1-(2,6-difluoro-phenyl)-methanone Trifluoroacetic Acid Salt.

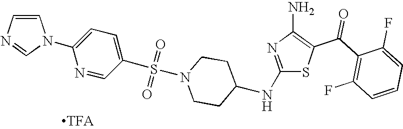

The title compound was prepared as follows. 1-{4-Amino-2-[1-(6-chloro-pyridine-3-sulfonyl)-piperidin-4-ylamino]-thiazol-5-yl}1-(2,6-difluoro-phenyl)-methanone (Example F21; 0.25 g, 0.50 mmol) and imidazole (0.68 g, 10 mmol) were ground together in a mortar and heated in a melt at 140° C. for 20 minutes. After allowing to cool, the solid was dissolved in ethyl acetate and washed with 0.1N NaOH. The organic layer was separated and concentrated. Preparative HPLC purification provided 0.22 g of product as a white power in 75% yield.

¹H NMR (CD₃OD): δ 9.80 (s, 1H), 9.02 (d, 1H, J=2.2 Hz), 8.50 (dd, 1H, J=2.2, 8.4 Hz), 8.44 (s, 1H), 8.16 (d, 1H, J=8.4 Hz), 7.80 (s, 1H), 7.44 (m, 1H), 7.00 (m, 2H), 3.76 (m, 3H), 2.76 (m, 2H), 2.12 (m, 2H), 1.68 (m, 2H). HRMALDIMS. Calcd for C₂₃H₂₂F₂N₇O₃S₂ (MH⁺): 546.1188. Found: 546.1202 Anal. Calcd for C₂₃H₂₁F₂N₇O₃S₂.1.5 TFA: C, 43.57; H, 3.16; N, 13.68; S, 8.95. Found: C, 43.53; H, 3.40; N, 13.70; S, 8.85.

### Example G26

### 1-(4-Amino-2-{1-[6-(2-methyl-imidazol-1-yl)-pyridine-3-sulfonyl]-piperidin-4-ylamino}-thiazol-5-yl)-1-(2,6-difluoro-phenyl)-methanone.

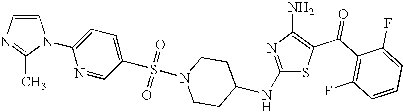

Prepared in a manner similar to that for Example G25.

¹H NMR (CD₃OD): δ 8.94 (d, 1H, J=2.5 Hz), 8.40 (dd, 1H, J=1.8, 8.2 Hz), 7.98 (d, 1H, J=5.5 Hz), 7.92 (d, 1H, J=8.2 Hz), 7.60 (d, 1H, J=1.8 Hz), 7.32 (m, 1H), 6.92 (m, 1H), 3.65 (m, 2H), 3.60 (br, 1H), 2.82 (s, 3H), 2.64 (m, 2H), 2.06 (m, 2H), 1.60 (m, 2H). HRMALDIMS. Calcd for C₂₄H₂₄F₂N₇O₃S₂ (MH⁺): 560.1345. Found: 560.1334.

### Example G27

### 1-(4-Amino-2-{1-[6-(4-methyl-imidazol-1-yl)-pyridine-3-sulfonyl]-piperidin-4-ylamino}-thiazol-5-yl)-1-(2,6-difluoro-phenyl)-methanone Hydrochloride.

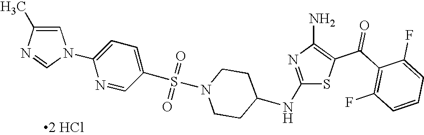

Prepared in a similar manner to that for Example G25 from 1-{4-amino-2-[1-(6-chloro-pyridine-3-sulfonyl)-piperidin-4-ylamino]-thiazol-5-yl}-1-(2,6-difluoro-phenyl)-methanone (Example F21) and 3-methylimidazole. Purification via preparative HPLC (Solvent system: A. 25 mM (NH₄)H₂PO₄/20 mM Et₃N in H₂O at pH3 adjusted with H₃PO₄; B.CH₃CN. Gradient: from 20% B to 60% B in 20 min. at a flow rate of 20 ml/min.) and treatment of fractions with excess aqueous HCl prior to lyophilization led to isolation of this compound as the major product in 75% yield.

¹H NMR (CD₃OD): δ 9.74 (s, 1H), 8.88 (d, 1H, J=2.2 Hz), 8.40 (dd, 1H, J=2.0, 8.0 Hz), 8.10 (s, 1H), 8.02 (d, 1H, J=8.0 Hz), 7.50 (m, 1H), 7.00 (m, 2H), 3.82 (br, 1H), 3.68 (m, 2H), 2.68 (m, 2H), 2.38 (s, 3H), 2.00 (m, 2H), 1.60 (m, 2H). HRMALDIMS. Calcd for C₂₄H₂₄F₂N₇O₃S₂ (MH⁺): 560.1345. Found: 560.1338. Anal. Calcd for C₂₄H₂₃F₂N₇O₃S₂.2.5 HCl.1.0 H₂O: C, 43.10; H, 4.14; N, 14.66; S, 9.59. Found: C, 43.25; H, 4.40; N, 14.69; S, 9.39.

### Example G28

### 1-(4-Amino-2-{1-[6-(5-methyl-imidazol-1-yl)-pyridine-3-sulfonyl]-piperidin-4-ylamino}-thiazol-5-yl)-1-(2,6-difluoro-phenyl)-methanone Hydrochloride.

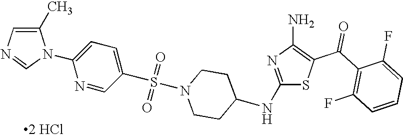

The title compound was obtained as a minor product from the preparation of Example G27 in 10% yield, after HPLC purification.

¹H NMR (CD₃OD): δ 9.50 (s, 1H), 9.10 (d, 1H, J=2.0 Hz), 8.54 (dd, 1H, J=2.0, 8.2 Hz), 8.06 (d, 1H, J=8.2 Hz), 7.60 (m, 2H), 7.16 (m, 2H), 4.00 (br, 1H), 3.82 (m, 2H), 2.82 (m, 2H), 2.60 (s, 3H), 2.14 (m, 2H), 1.74 (m, 2H). LC-ESIMS (MH⁺): 560. Anal. Calcd for C₂₄H₂₃F₂N₇O₃S₂.2.0 HCl.1.0 H₂O: C, 44.31; H, 4.18; N, 15.07; S, 9.86. Found: C, 44.16; H, 4.34; N, 14.99; S, 10.12.

### Example G29

### 1-(4-Amino-2-{1-[4-(3R,5S-dimethyl-piperazin-1-yl)-benzenesulfonyl]-piperidin-4-ylamino}-thiazol-5-yl)-1-(2,6-difluoro-phenyl)-methanone.

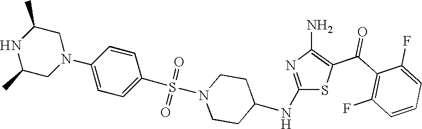

The title compound was prepared as follows. To a solution of 1-{4-amino-2-[1-(4-fluoro-benzenesulfonyl)-piperidin-4-ylamino]-thiazol-5-yl}-1-(2,6-difluoro-phenyl)-methanone (Example F17; 250 mg, 0.50 mmol) in DMSO (5 ml) were added anhydrous K₂CO₃ (139 mg, 1.00 mmol) and cis-2,6-dimethyl-piperazine (86 mg , 0.75 mmol). The mixture was heated to 120° C. for 48 h, allowed to cool to ambient temperature, and diluted with H₂O (10 ml). The yellow solid was collected by filtration, rinsed with H₂O, and purified via preparative TLC with 10% MeOH/CH₂Cl₂ to provide 48 mg of yellow powder in 16% yield.

¹H NMR (DMSO-d₆): δ 7.88 (bs, 2H), 7.42-7.32 (m, 3H), 7.05 (dd, 2H, J=7.8, 7.9 Hz), 6.95 (d, 2H, J=9.0 Hz), 3.72-3.62 (m, 2H), 3.38-3.26 (m, 3H), 2.78-2.68 (m, 2H), 2.26-2.16 (m, 2H), 1.88-1.74 (m, 2H), 1.42-1.32 (m, 2H), 0.94 (d, 6H, J=6.2 Hz). HRMALDIMS. Calcd. for C₂₇H₃₃F₂N₆O₃S₂ (MH⁺): 591.2018. Found: 591.1998. Anal. Calcd. for C₂₇H₃₂F₂N₆O₃S₂.0.6 H₂O: C, 53.91; H, 5.56; N, 13.64; S, 10.43. Found: C, 53.72; H, 5.63; N, 13.64; S, 10.43.

### Example G30

### 1-{4-Amino-2-[1-(4-imidazol-1-yl-benzenesulfonyl)-piperidin-4-ylamino]-thiazol-5-yl}-1-(2,6-difluoro-phenyl)-methanone.

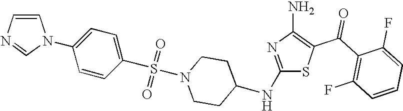

The title compound was prepared as follows. To a solution of 1-{4-amino-2-[1-(4-fluoro-benzenesulfonyl)-piperidin-4-ylamino]-thiazol-5-yl}-1-(2,6-difluoro-phenyl)-methanone (Example F17; 250 mg, 0.503 mmol) in DMSO (2 ml) were added imidazole (0.41 g, 0.60 mmol), and NaH (0.24 g, 1.0 mmol). The mixture was heated at 120° C. for 3 hours, allowed to cool to ambient temperature, and quenched with ice-cold H₂O (4 ml). The resultant precipitate was collected by filtration, rinsed with water and dried under vacuum to give 63 mg of a yellow powder in 22% yield.

¹H NMR (CD₃OD): δ 8.30 (s, 1H), 7.51 (s, 1H), 7.48-7.34 (m, 1H), 7.22 (s, 1H), 7.00 (dd, 2H, J=7.3, 8.4 Hz), 2.64 (dd, 2H, J=10.2, 10.3 Hz), 2.08 (d, 2H, J=10.5 Hz), 1.70-1.56 (m, 2H). HRESIMS Calcd. for C₂₄H₂₃F₂N₆O₃S₂ (MH⁺): 545.1241. Found: 545.1237 Anal. Calcd. for C₂₄H₂₂F₂N₆O₃S₂.1.5 H₂O: C, 50.43; H, 4.41; N, 14.70; S, 11.20. Found: C, 50.27; H, 4.16; N, 14.42; S, 11.23.

### Example G31

### 1-(4-Amino-2-{1-[4-(3,3-dimethyl-piperazin-1-yl)-benzenesulfonyl]-piperidin-4-ylamino}-thiazol-5-yl)-1-(2,6-difluoro-phenyl)-methanone.

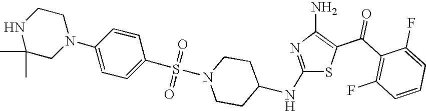

The title compound was prepared in manner similar to that used in preparation of Example G29 from 1-{4-amino-2-[1-(4-fluoro-benzenesulfonyl)-piperidin-4-ylamino]-thiazol-5-yl}-1-(2,6-difluoro-phenyl)-methanone (Example F17) and 2,2-dimethylpiperazine (Bφgesφ, et al., J. Med. Chem., Vol. 38, pp. 4380-4392 (1995)). Column chromatography with 10% MeOH/ CH₂Cl₂ gave a white solid in 19% yield.

¹H NMR (CD₃OD): δ 7.58 (d, 2H, J=9.0 Hz), 7.48-7.38 (m, 1H), 7.08-6.98 (m, 4H), 3.16 (s, 2H), 3.10 (dd, 2H, J=5.0, 5.6 Hz), 2.05 (d, 2H, J=13.0 Hz), 1.60-1.46 (m, 2H), 1.20 (s, 6H). HRESIMS. Calcd. for C₂₇H₃₃F₂N₆O₃S₂ (MH⁺): 591.2023. Found: 591.2029. Anal. Calcd. for C₂₇H₃₂F₂N₆O₃S₂.1.1 H₂O: C, 53.12: H, 5.65; N, 13.77; S, 10.50. Found: C, 52.86; H, 5.67; N, 13.61; S, 10.40.

### Example G32

### 1-{4-Amino-2-[1-(1-{6-[(2-dimethylamino-ethyl)-methyl-amino]-pyridin-3-yl}-methanoyl)-piperidin-4-ylamino]-thiazol-5-yl}-1-(2,6-difluoro-phenyl)-methanone Trifluoroacetic Acid Salt.

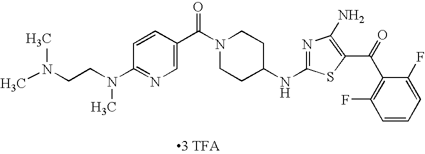

The title compound was prepared in a manner similar to that for Example G1 from 1-(4-amino-2-{1-[1-(6-chloro-pyridin-3-yl)-methanoyl]-piperidin-4-ylamino}-thiazol-5-yl)-1-(2,6-difluoro-phenyl)-methanone (Example C9) and N, N, N′-trimethyl-ethane-1,2-diamine. Purification via preparative HPLC provided 35 mg of white solid in 19% yield.

¹H NMR (DMSO-d₆): δ 9.53 (br, 1H), 8.82 (br, 1H), 8.20 (d, 1H, J=2.0 Hz), 8.06 (br, 1H), 7.62 (dd, 1H, J=2.0, 8.8 Hz), 7.50 (m, 1H), 7.16 (dd, 2H, J=7.8, 8.0 Hz), 6.73 (d, 1H, J=8.8 Hz), 4.10-3.90 (m, 2H), 3.95 (t, 2H, J=6.5 Hz), 3.31 (t, 2H, J=6.5 Hz), 3.10 (m, 1H), 3.03 (s, 3H), 2.86 (s, 6H), 2.00-1.85 (m, 2H), 1.50-1.33 (m, 2H). HRMALDIMS: Calcd. For C₂₆H₃₂F₂N₇O₂S (MH⁺): 544.2301. Found: 544.2289. Anal. Calcd. for C₂₆H₃₁F₂N₇O₂S.2.9 TFA: C, 43.69; H, 3.91; N, 11.21; S, 3.67. Found: C, 43.44; H, 5.75; N, 11.29; S, 3.67.

### Example G33

### (4-Amino-2-{1-[2-(3,5-dimethyl-piperazine-1-yl)-pyrimidine-5-sulfonyl]-piperidine-4-ylamino}-thiazol-5-yl)-(2,6-difluoro-phenyl)-methanone Hydrochloride Salt.

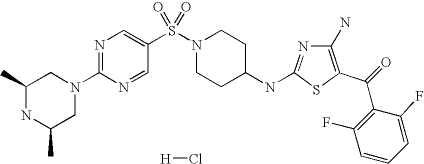

The title compound was prepared in a manner analogous to that used in Example G1 from (4-amino-2-{1-[2-(4-methyl-piperazin-1-yl)-pyrimindin-5-sulfonyl]-piperidin-4-ylamino}thiazol-5-yl)-(2,6-difluoro-phenyl)-methanone (Example F47) and cis-2,6-dimethyl piperazine to provide a pale white solid in 33% yield.

¹H NMR (DMSO-d₆): δ 9.45 (bs, 1H), 9.02 (bs, 1H), 8.73 (s, 2H), 8.21 (bs, 1H), 7.61-7.51 (m, 1H), 7.22 (t, 2H, J=15.9 Hz), 4.92 (d, 2H, J=12.9 Hz), 3.91-3.78 (m, 1H), 3.58-3.32 (m, 4H), 3.18 (t, 2H, J=11.2 Hz), 2.82-2.61 (m, 2H), 2.09-1.88 (m, 2H), 1.68-1.53 (m, 2H), 1.35 (d, 6H, J=6.4 Hz). HRMALDIMS: C₂₅H₃₁F₂N₈O₃S₂ (MH⁺): 593.1929. Found: 593.1918. Anal. Calcd. For C₂₅H₃₀F₂N₈O₃S₂.3.35 HCl.0.50 EtOAc.1.00 H₂O: C, 41.74; H, 5.11; N, 114.42; S, 8.25. Found: C, 41.72; H, 5.11; N, 14.42; S, 8.25.

### Example G34

### (4-Amino-2-{1-[2-(4-methyl-piperazin-1-yl)-pyrimindine-5-sulfonyl]-piperidin-4-ylamino}-thiazol-5-yl)-(2,6-difluoro-phenyl)-methanone Hydrochloride Salt.

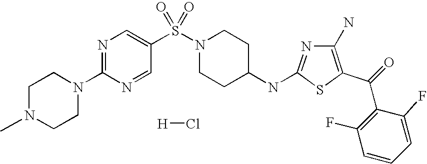

The title compound was prepared in a manner analogous to that used in Example G1 from (4-amino-2-{1-[2-(4-methyl-piperazin-1-yl)-pyrimindin-5-sulfonyl]-piperidin-4-ylamino}-thiazol-5-yl)-(2,6-difluoro-phenyl)-methanone (Example F47) and 1-methylpiperizine to provide a pale white solid in 33% yield.

¹H NMR (DMSO-d₆): δ 8.82 (bs, 1H), 8.71 (s, 2H), 8.02 (bs, 2H), 7.56-7.41 (m, 1H), 7.17 (t, 2H, J=15.9 Hz), 4.82 (d, 2H, J=14.6 Hz), 3.59-3.40 (m, 6H), 3.17-3.02 (m, 3H), 2.82 (d, 3H, J=4.4 Hz), 2.61-2.55 (m, 2H), 1.98-1.88 (m, 2H), 1.61-1.45 (m, 2H). HRMALDIMS: C₂₄H₂₉F₂N₈O₃S₂ (MH⁺): 579.1771. Found: 579.1750. Anal. Calcd. For C₂₄H₂₈F₂N₈O₃S₂.2.00 HCl.0.62 H₂O: C, 43.49; H, 4.75; N, 16.91; S, 9.68. Found: C, 43.49; H, 4.97; N, 16.71; S, 9.51.
Method H:

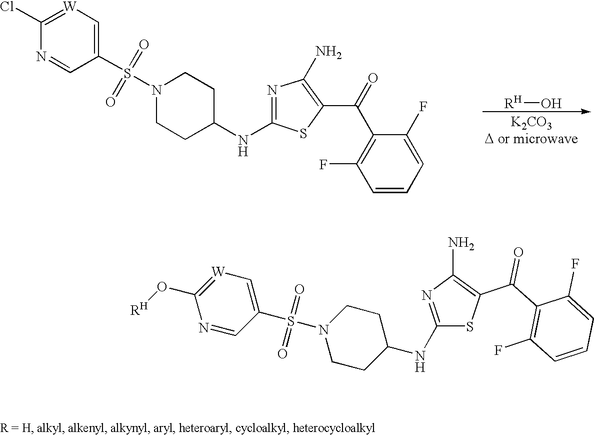

### Example H1

### 1-{4-Amino-2-[1-(6-hydroxy-pyridine-3-sulfonyl)-piperidin-4-ylamino]-thiazol-5-yl}-1-(2,6-difluoro-phenyl)-methanone Trifluoroacetic Acid Salt.

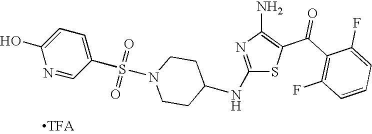

The title compound was prepared as follows. A mixture of 1-{4-amino-2-[1-(6-chloro-pyridine-3-sulfonyl)-piperidin-4-ylamino]-thiazol-5-yl}-1-phenyl-methanone (Example F21; 63 mg, 0.12 mmol), sat. sodium hydroxide (1 ml), and tert-butanol (1 ml) was heated for two 45 second intervals in a microwave oven (0.7 cu. ft., 800 watt). The mixture was allowed to cool, diluted with ethyl acetate (75 mL), washed with sat. NaHCO₃ (3×25 ml), dried over MgSO₄, filtered, and concentrated. Purification via preparative HPLC provided 15.0 mg of white powder in 25% yield.

¹H NMR (DMSO-d₆): δ 7.97 (d, 1H, J=2.3 Hz), 7.76 (dd, 1H, J=2.3, 9.0 Hz), 7.52-7.40 (m, 1H), 7.08-6.98 (m, 2H), 6.60 (d, 1H, J=9.0 Hz), 3.70-3.57 (m, 3H), 2.81-2.68 (m, 2H), 2.18-2.04 (m, 2H), 1.70-1.57 (m, 2H). HRMALDIMS. Calcd for C₂₀H₂₀F₂N₅O₄S₂ (MH⁺): 496.0919. Found: 496.0913 Anal. Calcd for C₂₀H₁₉F₂N₅O₄S₂.1.4 TFA: C, 41.80; H, 3.14; N, 10.69; S, 9.79. Found: C, 41.82; H, 3.48; N, 10.94; S, 9.83.

### Example H2

### 1-{4-Amino-2-[1-{6-morpholin-(4-yl-ethoxy)-pyridine-3-sulfonyl}-piperidin-4-ylamino]-thiazol-5-yl}-1-(2,6-difluoro-phenyl)-methanone Hydrochloride.

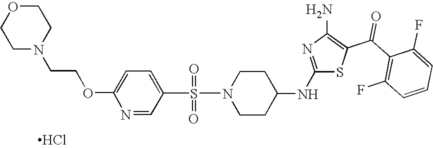

The title compound was prepared as follows. 1-{4-Amino-2-[1-(6-chloro-pyridine-3-sulfonyl)-piperidin-4-ylamino]-thiazol-5-yl}-1-phenyl-methanone (Example F21; 510 mg, 1.00 mmol), 4-(2-hydroxyethyl) morpholine (5.0 ml, 39 mmol), and potassium carbonate (500 mg, 3.62 mmol) were ground together in a mortar, transferred to a flask, and heated at 120° C. for 2 hours. The resultant mixture was allowed to cool, diluted with ethyl acetate, washed with water, dried over MgSO₄, filtered, and concentrated. Column chromatography with (58% NH₄OH)/MeOH/EtOAc (0.5/1/10) as eluant provided a white powder, which was taken up in EtOAc and washed with water, dried over Na₂SO₄, and concentrated. The resultant solid was dissolved in acetonitrile (25 ml), water (60 ml) and 38% HCl (0.5 ml) and lyophilized to give 0.33 g of yellow solid in 46% yield.

¹H NMR (DMSO-d₆): δ 8.50 (d, 1H, J=2.1 Hz), 7.98 (dd, 1H, J=2.1, 8.8 Hz), 7.52 (m, 1H), 7.11-6.86 (m, 3H), 4.10-3.42 (m, 15H), 2.68-2.53 (m, 2H), 2.04-1.92 (m, 2H), 1.68-1.48 (m, 2H). ESIMS (MH⁺): 609. Anal. Calcd for C₂₆H₃₀F₂N₆O₅S₂.2.80 HCl.0.30 H₂O: C, 43.60; H, 4.70; N, 11.73; S, 8.95. Found: C, 43.39; H, 4.99; N, 11.79; S, 8.64.

The following Examples from H3 through H16 were prepared in a manner similar to that for Example H2, from 1-{4-amino-2-[1-(6-chloro-pyridine-3-sulfonyl)-piperidin-4-ylamino]-thiazol-5-yl}-1-phenyl-methanone (Example F21)and corresponding alcohols and purified via either column chromatography or reversed phase preparative HPLC.

### Example H3

### 1-(4-Amino-2-{1-[6-(2-dimethylamino-ethoxy)-pyridine-3-sulfonyl]-piperidin-4-ylamino}-thiazol-5-yl)-1-(2,6-difluoro-phenyl)-methanone Hydrochloride.

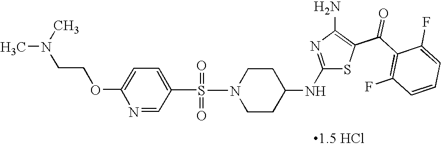

¹H NMR (CD₃OD): δ 8.60 (d, 1H, J=2.2 Hz), 8.10 (dd, 1H, J=1.2, 8.2 Hz), 7.44 (m, 1H), 7.04 (m, 3H), 4.82 (m, 2H), 3.68 (m, 5H), 3.04 (s, 3H), 2.64 (m, 2H), 2.12 (m, 2H), 1.68 (m, 2H). HRMALDIMS. Calcd for C₂₄H₂₉F₂N₆O₄S₂ (MH⁺): 567.1654. Found: 567.1658. Anal. Calcd for C₂₄H₂₈F₂N₆O₄S₂.1.5 HCl.0.50 H₂O: C, 45.73; H, 4.88; N, 13.33; S, 10.17. Found: C, 45.66; H, 4.98; N, 13.10; S8 10.22.

### Example H4

### 1-(4-Amino-2-{1-[6-(2-hydroxy-ethoxy)-pyridine-3-sulfonyl]-piperidin-4-ylamino}-thiazol-5-yl)-1-(2,6-difluoro-phenyl)-methanone.

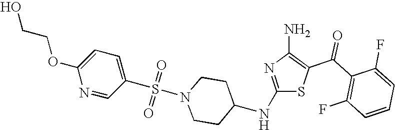

Purified via preparative HPLC.

¹H NMR (CD₃OD): δ 8.40 (d, 1H, J=2.0 Hz), 7.88 (dd, 1H, J=2.0, 8.0 Hz), 7.30 (m, 1H), 6.90 (m, 3H), 4.36 (t, 2H, J=5.6 Hz), 3.78 (t, 2H, J=5.6 Hz), 3.52 (m, 3H), 2.50 (m, 2H), 1.94 (m, 2H), 1.50 (m, 2H). HRMALDIMS. Calcd for C₂₂H₂₄F₂N₅O₅S₂ (MH⁺): 540.1181. Found: 540.1184.

### Example H5

### 1-(4-Amino-2-{1-[6-(2-pyrrolidin-1-yl-ethoxy)-pyridine-3-sulfonyl]-piperidin-4-ylamino}-thiazol-5-yl)-1-(2,6-difluoro-phenyl)-methanone Trifluoroacetic Acid Salt.

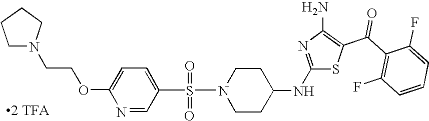

Purified via preparative HPLC.

1H NMR (CD₃OD): δ 8.53 (d, 1H, J=2.5 Hz), 8.00 (dd, 1H, J=2.5, 8.8 Hz), 7.50-7.38 (m, 1H), 7.06-6.97(m, 3H), 5.58 (t, 2H, J=5.7 Hz), 3.70-3.61 (m, 3H), 3.00-2.92 (m, 2H), 2.78-2.60 (m, 6H), 2.13-2.02 (m, 2H), 1.89-1.81 (m, 4H), 1.70-1.53 (m, 2H). HRMALDIMS. Calcd for C₂₆H₃₁F₂N₆O₄S₂ (MH⁺): 593.1811. Found: 593.1787. Anal. Calcd for C₂₆H₃₀F₂N₆O₄S₂.1.9 TFA: C, 44.22; H, 3.97; N, 10.38; S, 7.92. Found: C, 44.04; H, 4.16; N, 10.55; S, 7.99.

### Example H6

### 1-[4-Amino-2-{1-[6-(2-piperidin-1-yl-ethoxy)-pyridine-3-sulfonyl]-piperidin-4-ylamino}-thiazol-5-yl)-1-(2,6-d ifluoro-phenyl)-methanone Dihydrochloride.

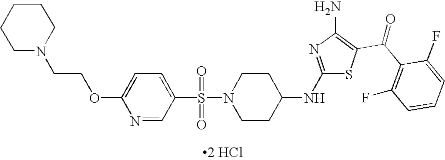

¹H NMR (CD₃OD): δ 8.60 (d, 1H, J=2.6 Hz), 8.09 (dd, 1H, J=2.6, 8.7 Hz,), 7.60-7.56 (m, 1H), 7.22-7.10 (m, 3H), 3.72-3.51 (m, 5H), 3.18-3.00 (m, 2H), 2.70-2.56 (m, 2H), 2.18-1.47 (m, 14H). HRMALDIMS. Calcd for C₂₇H₃₃F₂N₆O₄S₂ (MH⁺): 607.1967. Found: 607.1953. Anal. Calcd for C₂₇H₃₂F₂N₆O₄S.2.0 HCl: C, 47.71; H, 5.04; N, 12.37; S, 9.44. Found: C, 47.46; H, 5.34; N, 12.26; S, 9.27.

### Example H7

### 1-[4-Amino-2-{1-[6-(1-methyl-piperidin-3RS-ylmethoxy)-pyridine-3-sulfonyl]-piperidin-4-ylamino}-thiazol-5-yl)-1-(2,6-difluoro-phenyl)-methanone Trifluoroacetic Acid Salt.

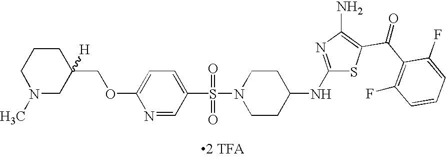

Purified via preparative HPLC.

¹H NMR (CD₃OD): δ 8.58 (d, 1H, J=2.5 Hz), 8.03 (dd, 1H, J=2.5, 8.7 Hz), 7.52-7.39 (m, 1H), 7.08-6.97 (m, 3H), 4.56-4.43 (m, 1H), 4.38-4.29 (m, 1H), 3.72-3.63 (m, 3H), 3.58-3.50 (m, 2H), 3.00-2.86 (m, 5H), 2.70-2.53 (m, 2H), 2.44-2.30 (m, 1H), 2.12-1.93 (m, 2H), 1.91-1.73 (m, 1H), 1.70-1.56 (m, 2H), 1.53-1.38 (m, 2H). ESIMS (MH⁺): 607. Anal. Calcd for C₂₇H₃₂F₂N₆O₄S₂.2.4TFA: C, 43.38; H, 3.94; N, 9.55; S, 7.28. Found: C, 43.26; H, 4.10; N, 9.72; S, 7.36.

### Example H8

### 1-(4-Amino-2-{1-[6-(pyridin-3-ylmethoxy)-pyridine-3-sulfonyl]-piperidin-4-ylamino}-thiazol-5-yl)-1-(2,6-difluoro-phenyl)-methanone Trifluoroacetic Acid Salt.

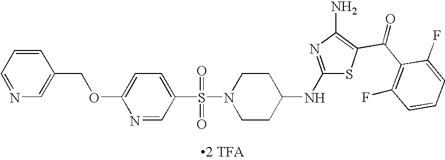

Purified via preparative HPLC.

¹H NMR (CD₃OD): δ 9.00 (s, 1H), 8.81 (d, 1H, J=5.8 Hz), 8.68 (d, 1H, J=7.7 Hz), 8.60-8.56 (m, 2H), 8.12-8.00 (m, 2H), 7.50-7.39 (m, 1H), 7.17-6.98 (m, 2H), 5.71 (s, 2H), 3.75-3.58 (m, 3H), 2.68-2.57 (m, 2H), 2.17-2.02 (m, 2H), 1.71-1.54 (m, 2H). ESIMS (MH⁺): 587. Anal. Calcd for C₂₆H₂₄F₂N₆O₄S₂.2.5TFA: C, 42.71; H, 3.06; N, 9.64; S, 7.36. Found: C, 42.60; H, 3.24; N, 9.73; S, 7.34.

### Example H9

### 1-(4-Amino-2-{1-[6-(2-imidazol-1-yl-ethoxy)-pyridine-3-sulfonyl]-piperidin-4-ylamino}-thiazol-5-yl)-1-(2,6-difluoro-phenyl)-methanone Hydrochloride.

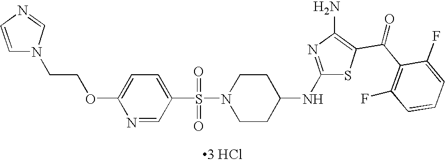

Purified via preparative HPLC and fractions treated with HCl prior to lyophilization.

¹H NMR (CD₃OD): δ 9.08 s1H), 8.54 (d, 1H, J=2.5 Hz), 8.04 (dd, 1H, J=2.5, 8.7 Hz), 7.76 (t, 1H, J=1.7 Hz), 7.61-7.49 (m, 2H), 7.17-6.98 (m, 3H), 4.90-4.70 (m, 4H), 3.74-3.65 (m, 3H), 2.70-2.56 (m, 2H), 2.18-2.03 (m, 2H), 1.73-1.58 (m, 2H). ESIMS (MH⁺): 590. Anal. Calcd for C₂₅H₂₅F₂N₇O₄S₂.3.25 HCl: C, 42.40; H, 4.02; N, 13.85; S, 9.06. Found: C, 42.12; H, 4.17; N, 13.63; S, 8.96.

### Example H10

### 1-(4-Amino-2-{1-[6-(1-methyl-piperidin-3R-ylmethoxy)-pyridine-3-sulfonyl]-piperidin-4-ylamino}-thiazol-5-yl)-1-(2,6-difluoro-phenyl)-methanone.

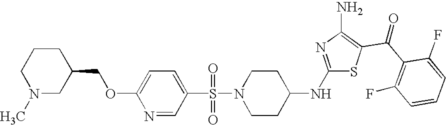

The title compound was prepared in a manner analogous to that for Example H2. 1-{4-Amino-2-[1-(6-chloro-pyridine-3-sulfonyl)-piperidin-4-ylamino]-thiazol-5-yl}-1-phenyl-methanone (Example F21) and crude (1-methyl-piperidin-3R-yl)-methanol (International Patent Publication WO99/21855) gave, after column chromatography with 0.5% (58% NH₄OH)/6% MeOH/CH₂Cl₂), a yellow foam in 84% yield.

¹H NMR (DMSO-d₆): δ 8.50 (d, 1H, J=2.2 Hz), 8.06-7.94 (m, 3H), 7.48 (ddd, 1H, J=1.8, 6.7, 8.4 Hz), 7.14 (dd, 2H, J=7.6, 8.1 Hz), 7.02 (d, 1H, J=8.8 Hz), 4.28 (dd, 1H, J=5.9, 10.6 Hz), 4.18 (dd, 1H, J=7.4, 10.6 Hz), 3.48 (d, 2H, J=11.5 Hz), 2.80 (d, 1H, J=9.4 Hz), 2.68-2.52 (m, 2H), 2.18 (s, 3H), 2.02-1.42 (m, 10H), 0.98 (m, 1H). HRMALDIMS. Calcd. for C₂₇H₃₃F₂N₆O₄S₂ (MH⁺): 607.1967. Found: 607.1960. Anal. Calcd. for C₂₇H₃₂F₂N₆O₄S₂.1.1 H₂O.0.4 CHCl₃: C, 48.81; H, 5.17; N, 12.46; S, 9.51. Found: C, 48.43; H, 4.92; N, 12.25; S, 9.23.

### Example H11

### 1-(4-Amino-2-{1-[6-(1-methyl-piperidin-3S-ylmethoxy)-pyridine-3-sulfonyl]-piperidin-4-ylamino}-thiazol-5-yl)-1-(2,6-difluoro-phenyl)-methanone Dihydrochloride.

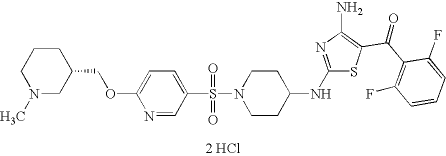

The starting materials were prepared as follows:
(S)-Ethyl nipecotate

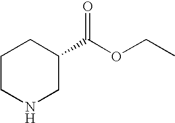

Obtained via resolution of racemic ethyl nipecotate as described by Abele, et al., Helv. Chim. Acta 82, 1539-1558 (1999). The (S)-ethyl nipecotate liberated from the D-tartrate salt was analyzed for optical purity as the 2S-naphthyl-ethyl urea derivative as described by Magnus, et al., J. Org. Chem. 56, 1166-1170 (1991) compared by NMR to the mixture from racemate. Used without any further purification.
Ethyl N-carbethoxy-pipenidine-3S-carboxylate

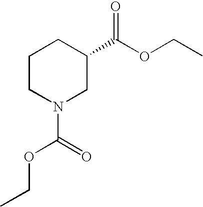

(S)-Ethyl nipecotate (1.02 g, 6.51 mmol) and N-methylmorpholine (0.752 mL, 6.84 mmol) in CHCl₃ (10 mL) at 0° C. was treated with ethyl chloroformate (0.641 mL, 6.70 mmol) and allowed to slowly warm to ambient temperature overnight. The resultant mixture stirred with 10% aq KHSO₄ (15 mL). The organic layer was separated and washed with sat. NaHCO₃ (10 mL), dried over Na₂SO₄ and evaporated to give 1.49 g of a yellow oil in 100% yield, which displayed an identical NMR spectrum to that reported for the R isomer (International Patent Publication No. WO 99/21855) and was used without further purification.
(1-Methyl-piperidin-3S-yl)-methanol

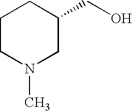

As described for the R isomer in International Publication No. WO 99/21855, ethyl N-carbethoxy-piperidine-3S-carboxylate was reduced with LiAlH₄ in THF to provide 562 mg of a yellow oil in 67% yield, which had an NMR spectrum that matched the R-isomer and was used without further purification.
1-(4-Amino-2-{-[6-(1-methyl-piperidin-3S-ylmethoxy)-pyridine-3-sulfonyl]-piperidin-4-ylamino}-thiazol-5yl)-1-(2,6-difluoro-phenyl)-methanone

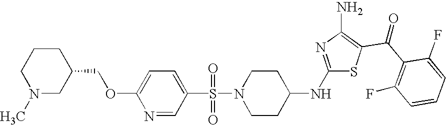

The title compound was prepared in a manner similar to that for Example H2. 1-{4-Amino-2-[1-(6-chloro-pyridine-3-sulfonyl)-piperidin-4-ylamino]-thiazol-5-yl}-1-phenyl-methanone (Example F21) and crude (1-methyl-piperidin-3S-yl)-methanol furnished, after radial chromatography with a stepwise gradient of 0.5% (58% NH₄OH)/2% MeOH/CHCl₃ to 1% (58% NH₄OH)/10% MeOH/CHCl₃, 200 mg of a hard yellow foam in 50% yield, and precipitated from CHCl₃/hexane as a white solid, mp determination attempt led to decomp. >110° C.

8.00 (dd, 1H, J=2.6, 8.8 Hz), 7.90 (s, 1H), 7.43 (ddd, 1H, J=6.5, 8.3, 8.8 Hz), 7.02 (ddd, 2H, J=0.9, 1.3, 8.3 Hz), 6.97 (d, 1H, J=8.8 Hz), 4.35 (dd, 1H, J=5.6, 10.7 Hz), 4.23 (dd, 1H, J=7.4, 10.7 Hz), 3.02 (d, 1H, J=11.3 Hz), 2.85 (d, 1H, J=11.3 Hz), 2.63 (dd, 2H, J=3.2, 14.1 Hz), 2.30 (s, 3H). FTIR (KBr): 3411, 2937, 1618, 1589, 1547, 1463, 1360, 1168, 1002 cm⁻¹. LCCIMS: (MH⁺) 607.10. Anal. Calcd. for C₂₇H₃₂F₂N₆O₄S₂.1.5 MeOH: C, 52.28; H, 5.85; N, 12.83; S, 9.79. Found: C, 52.18; H, 5.59; N, 12.57; S, 9.79.

The title compound of this Example H 11 was prepared as follows. To a suspension of 1-(4-amino-2-{1-[6-(1-methyl-piperidin-3S-ylmethoxy)-pyridine-3-sulfonyl]piperidin-4-ylamino}-thiazol-5-yl)-1-(2,6-difluoro-phenyl)-methanone (0.80 g, 1.32 mmol) in MeOH (10 ml) at room temperature was added a solution of 4N HCl (0.824 ml, 3.29 mmol) in dioxane. The resulting solution was stirred for 0.5 h and concentrated in vacuo to afford a cream foam in 100% yield.

¹H NMR (CD₃OD): δ 8.58 (1H, d, J=2.4 Hz), 8.04 (1H, dd, J=2.5, 8.8 Hz), 7.14 (2H, dd, J=8.1, 8.2 Hz), 7.00 (1H, d, J=8.8 Hz), 4.48 (1H, dd, J=4.5, 11.0 Hz), 4.32 (1H, dd, J=7.1, 11.1 Hz), 2.92 (3H, s). Anal. Calcd. for C₂₇H₃₂F₂N₆O₄S₂.2HCl.1.4 H₂O: C, 45.64; H, 5.58; N, 11.40; Cl, 9.62; S, 8.70. Found: C, 45.70; H, 5.47; N, 11.03; Cl, 10.00; S, 8.42.

### Example H12

### 1-(4-Amino-2-{1-[6-(1-methyl-pyrrolidin-2S-ylmethoxy)-pyridine-3-sulfonyl]-piperidin-4-ylamino}-thiazol-5-yl)-1-(2,6-difluoro-phenyl)-methanone.

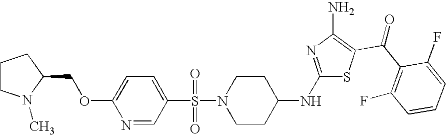

The title compound was prepared in a manner analogous to that for Example H2. 1-{4-Amino-2-[1-(6-chloro-pyridine-3-sulfonyl)-piperidin-4-ylamino]-thiazol-5-yl}-1-phenyl-methanone (Example F21) and (S)-(−)-2-hydroxymethyl-1-methylpyrrolidine gave, after column chromatography with 1% (58% NH₄OH)/10% MeOH/CH₂Cl₂, a yellow foam in 49% yield.

¹H NMR (CD₃OD): δ 8.54 (d, 1H, J=2.4 Hz), 7.89 (dd, 1H, J=2.5, 8.8 Hz), 7.48-7.36 (m, 1H), 4.4 (d, 2H, J=5.4 Hz), 3.15-3.08 (m, 1H), 2.48 (s, 3H). HRESIMS. Calcd. for C₂₆H₃₁F₂N₆O₄S₂ (MH⁺): 593.1816. Found: 593.1812. Anal. Calcd. for C₂₆H₃₀F₂N₆O₄S₂.0.5H₂O: C, 51.90; H, 5.19; N, 13.97; S, 10.66. Found: C, 51.50; H, 5.18; N, 13.71; S, 10.36.

### Example H13

### 1-(4-Amino-2-{1-[6-(2-dimethylamino-1RS-methyl-ethoxy)-pyridine-3-sulfonyl]-piperidin-4-ylamino}-thiazol-5-yl)-1-(2,6-fifluoro-phenyl)-methanone Dihydrochloride.

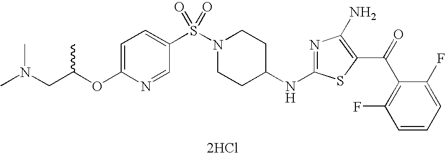

¹H NMR (DMSO-d₆): 3 8.88 (br, 1H), 8.54 (d, 2H, J=2.2 Hz), 8.09-7.91 (m, 3H), 7.54-7.42 (m, 1H), 7.17-7.02 (m, 2H), 7.07 (d, 1H, J=8.8 Hz), 5.63 (m, 1H), 3.58-3.33 (m, 5H), 2.85-2.74 (m, 6H), 2.64-2.59 (m, 2H), 1.98-1.95 (m, 2H), 1.61-1.48 (m, 2H), 1.38 (d, 3H, J=6.2 Hz). ESIMS (MH⁺): 581. Anal. Calcd. for C₂₅H₃₀F₂N₆O₄S₂.2.50 HCl.0.90 H₂O: C, 43.64; H, 5.21; N, 12.00; S, 9.26. Found: C, 43.64; H, 5.03; N, 12.21; S, 9.26.

### Example H14

### 1-(4-Amino-2-{1-[6-(1-methyl-piperidin-4-yloxy)-pyridine-3-sulfonyl]-piperidin-4-ylamino}-thiazol-5-yl)-1-(2,6-difluoro-phenyl)-methanone Hydrochloride.

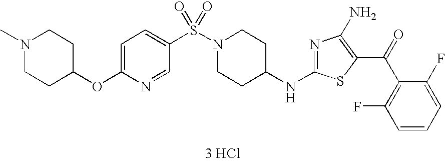

¹H NMR (DMSO-d₆): δ 8.80 (br, 1H), 8.53 (m, 1H), 8.09-7.90 (m, 3H), 7.48 (m, 1H), 7.18 (t, 2H, J=7.9 Hz), 7.05 (m, 1H), 5.43 9s, 1H), 5.28 (m, 1H), 3.54-3.42 (m, 3H), 3.34 (m, 1H), 3.21-3.12 (m, 2H), 2.78-2.70 (m, 3H), 2.64-2.54 (m, 2H), 2.32-1.87 (m, 6H), 1.54(m, 2H). ESIMS (MH⁺): 593. Anal. Calcd. for C₂₆H₃₀F₂N₆O₄S₂.3.5 HCl.2.40 H₂O: C, 40.90; H, 5.06; N, 11.01; S, 8.40. Found: C, 40.94; H, 5.26; N, 10.90; S, 8.46.

### Example H15

### 1-(4-Amino-2-{1-[6-(3-dimethylamino-propoxy)-pyridine-3-sulfonyl]-piperidin-4-ylamino}-thiazol-5-yl)-1-(2,6-difluoro-phenyl)-methanone Hydrochloride.

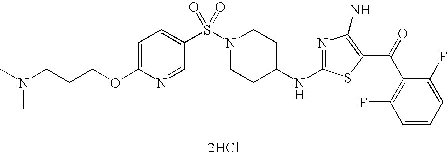

1H NMR (DMSO-d₆): δ 8.82 (br, 1H), 8.53 (d,1H, J=2.1 Hz), 8.08-7.90 (m, 3H), 7.50 (m, 1H), 7.15 (t, 2H, J=7.8 Hz), 7.02 (d, 1H, J=8.8 Hz), 4.39 (t, 2H, J=6.1 Hz), 3.56-3.40 (m, 3H), 3.22-3.13 (m, 2H), 2.65-2.58 (m, 2H), 2.22-2.12 (m, 2H), 1.99-1.88 (m, 2H), 1.55-1.46 (m, 2H). ESIMS (MH⁺): 581. Anal. Calcd. For C₂₅H₃₀F₂N₆O₄S₂.2.5 HCl.0.90 H₂O: C, 43.64; H, 5.03; N, 12.21; S, 9.32. Found: C, 43.61; H, 5.17; N, 12.24; S, 9.29.

### Example H16

### 1-(4-Amino-2-{1-[6-(1-methyl-piperidin-3RS-yloxy)-pyridin-3-sulfonyl]-piperidin-4-ylamino}-thiazol-5-yl)-1-(2,6-difluoro-phenyl)-methanone Hydrochloride.

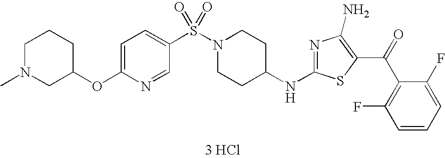

¹H NMR (DMSO-d₆): δ 8.82 (br, 1H), 8.53 (s, 1H), 8.11-7.90 (m, 3H), 7.49 (m, 1H), 7.15 (t, 2H, J=7.9 Hz), 7.05 (d, 1H, J=8.7 Hz), 5.54 (m, 1H), 3.65 (m, 1H), 3.58-3.22 (m, 4H), 2.98-2.87 (m, 2H), 2.73 (s, 3H), 2.65-2.58 (m, 2H), 2.08-1.88 (m, 4H), 1.78-1.72 (m, 2H), 1.58-1.48 (m, 2H). ESIMS (MH⁺): 593. Anal. Calcd. For C₂₆H₃₀F₂N₆O₄S₂.3.25 HCl.3.00 H₂O: C, 40.81; H, 5.17; N, 10.98; S, 8.38. Found: C, 40.80; H, 5.33; N, 10.92; S, 8.24.

### Example H17

### 1-(4-Amino-2-{1-[6-(2-dimethylamino-ethoxy)-pyridine-3-sulfonyl]-piperidin-4-ylamino}-thiazol-5-yl)-1-(2,6-dichloro-phenyl)-methanone Hydrochloride Salt

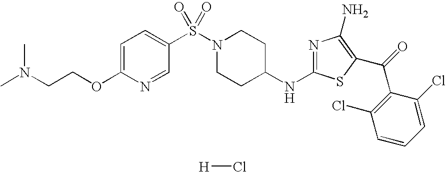

The title compound was prepared in a manner similar to that for Example H2 from 1-{4-amino-2-[1-(6-chloro-pyridine-3-sulfonyl)-piperidin-4-ylamino}-thiazol-5-yl)-1-(2,6-dichloro-phenyl)-methanone

¹H NMR (DMSO-d₆): δ 8.84 (bs, 1H), 8.60 (s, 2H), 8.18-8.10 (m, 1H), 7.96 (bs, 2H), 7.58-7.42 (m, 3H), 7.24 (d, 1H, J=8.8 Hz), 4.75 (t, 2H, J=5.0 Hz), 3.60-3.51 (m, 2H), 2.91 (S, 6H), 2.84 (m, 2H), 2.73-2.61 (m, 3H), 2.05-1.95 (m, 2H), 1.68-1.52 (m, 2H). HRMALDIMS: C₂₄H₂₉N₆O₄S₂Cl₂ (MH⁺): 599.1069. Found: 599.1093. Anal. Calcd. For C₂₄H₂₈N₆O₄S₂Cl₂.1.75 HCl.0.15 EtOAc.0.9 H₂O: C, 42.6; H, 4.77; N, 12.13; S, 9.26. Found: C, 42.66; H, 4.87; N, 12.08; S, 9.15.

### Example H18

### (4-Amino-2-{1-[6-(2-diethylamino-ethoxy)-pyridine-3-sulfonyl]-piperidin-4-ylamino}-thiazol-5-yl)-(2,6-difluoro-phenyl)-methanone Trifluoroacetic Acid Salt

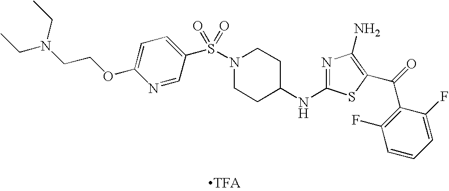

The title compound was prepared in a manner similar to that used to prepare the Example H2 from 1-{4-amino-2-[1-(6-chloro-pyridine-3-sulfonyl)-piperidin-4-ylamino]-thiazol-5-yl]-(2,6-difluoro-phenyl)-methanone (Example F21) and 2-diethylaminoethanol in 54% yield.

¹H NMR (CD₃OD): δ 8.70 (d, 1H, J=2.45), 8.20 (dd, 1H, J=2.4, 8.8 Hz), 7.46 (m, 1H), 7.25-7.10 (m, 3H), 4.904.77 (m, 2H), 3.92-3.80 (m, 5H), 3.52-3.43 (m, 4H), 2.63 (m, 2H), 2.15 (m, 2H), 1.70 (m, 2H), 1.48 (t, 6H). ESIMS (MH⁺): 595. Anal. Calcd for C₂₆H₃₂F₂N₆O₄S₂.1.5 TFA.0.70 H₂O: C, 47.43; H, 5.28; N, 12.76; S, 9.74. Found: C, 47.32; H, 5.41; N, 12.74; S, 9.59.

### Example H19

### (4-Amino-2-{1-[6-(2-isopropylamino-ethoxy)-pyddine-3-sulfonyl]-piperidin-4-ylamino}-thiazol-5-yl)-(2,6-difluoro-phenyl)-methanone Hydrochloride Salt.

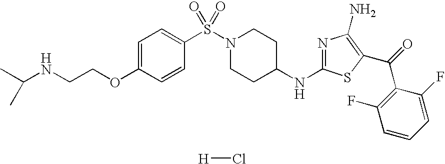

The starting material was prepared as follows:
(4-Amino-2-{1-[4-(2,2-dimethoxy-ethoxy)-benzenesulfonyl]-piperidin-4-ylamino}-thiazol-5-yl)-(2,6-difluorophenyl)-methanone.

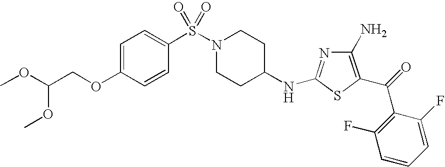

The above intermediate was prepared in a manner similar to that for Example H2, from 1-{4-amino-2-[1-(6-chloro-pyridine-3-sulfonyl)-piperidin-4-ylamino]-thiazol-5-yl-phenyl-methanone (Example F21) and glycolaldehyde dimethyl acetal gave, after column chromatography (EtOAc:Hexane=2:1), a pale white solid in 93% yield.

¹H NMR (DMSO-d₆): δ 8.80 (bs, 1H), 8.55 (s, 1H), 8.08-7.95 (m, 3H), 7.50-7.23 (m, 1H), 7.18-7.00 (m, 3H), 4.74-4.65 (m, 1H), 4.45-4.37 (m, 3H), 3.51-3.38 (m, 2H), 3.25 (s, 6H), 2.68-2.52 (m, 2H), 1.98-1.84 (m, 2H), 1.57-1.42 (m, 2H). LCESIMS: (MH⁻): 582.0.
(4-{4-[4-Amino-5-(2,6-difluoro-benzoyl)-thiazol-2-ylamino]-piperidine-1-sulfonyl}-phenoxy)-acetaldehyde

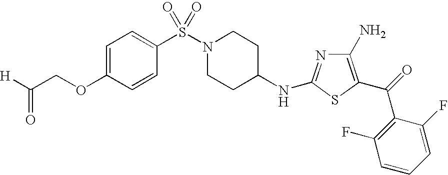

To a solution of (4-amino-2-{1-[4-(2,2-dimethoxy-ethoxy)-benzenesulfonyl]-piperidin-4-ylamino}-thiazol-5-yl)-(2,6-difluoro-phenyl)-methanone (0.070 g, 0.12 mmol) in acetone (4 ml) was added trifluoro-methanesulfonic acid (21 uml, 0.24 mmol) at −10° C. The reaction solution was stirred for 3 hours and then stored at 4° C. overnight. To the reaction solution was added additional amount of trifluoro-methanesulfonic acid (21 ul, 0.24 mmol) and 2 drops of water. The reaction mixture was then refluxed for 3 hours, cooled and diluted with ethyl acetate. The resultant solution was washed with NaHCO₃, brine, dried over MgSO₄, filtered and concentrated to give crude product, which was used without further purification.

LCESIMS (MH⁺): 538.

The title compound of this Example H19 was prepared in a manner analogous to that for Example J6 from (4-{4-[4-amino-5-(2,6-difluoro-benzoyl)-thiazol-2-ylamino]-piperidine-1-sulfonyl}-phenoxy)-acetaldehyde and isopropylamine to give, after preparative HPLC purification, a white solid in 20% yield.

¹H NMR (DMSO-d₆): δ 9.08-8.80 (m, 3H), 8.62 (s, 1H), 8.18-8.02 (m, 2H), 7.55 (m, 1H), 7.10-7.25 (m, 3H), 4.70 (t, 2H, J=4.7 Hz), 3.58-3.45 (m, 6H), 2.69-2.61 (m, 2H), 2.08-1.90 (m, 2H), 1.30 (d, 6H, J=6.5 Hz). LCESIMS (MH⁺): 581.3. Anal. Calcd. For C₂₅H₃₀F₂N₆O₄S₂.2.90 HCl.0.20 EtOAc.3.00 H₂O: C, 41.87; H, 5.24; N, 11.36; S, 8.67. Found: C, 41.85; H, 5.12; N, 11.36; S, 8.54.

### Example H20

### (4-Amino-2-{1-[6-(2-tert-butylamino-ethoxy)-pyridine-3-sulfonyl]-piperidin-4-ylamino}-thiazol-5-yl)-(2,6-difluoro-phenyl)-methanone Hydrochloride Salt.

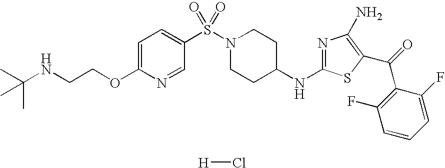

The title compound was prepared in a manner analogous to that for Example H19 from (4-{4-[4-amino-5-(2,6-difluoro-benzoyl)-thiazol-2-ylamino]-piperidine-1-sulfonyl}-phenoxy)-acetaldehyde (from Example H19) and tert-butylamine in 25% yield.

¹H NMR (DMSO-d₆): δ 8.82 (bs, 2H), 8.71 (s, 1H), 8.10-7.98 (m, 2H), 7.55-7.45 (m, 1H), 7.20-7.04 (m, 3H), 4.65 (t, 2H, J=4.80 Hz), 3.52-3.30 (m, 4H), 2.70-2.48 (m, 3H), 1.98-1.82 (m, 2H), 1.58-1.42 (m, 2H), 1.30 (s, 9H). HRMALDIMS: C₂₆H₃₃F₂N₆O₄S₂ (MH⁺): 595.1973. Found: 595.1968. Anal. Calcd. For C₂₆H₃₂F₂N₆O₄S₂.2.70 HCl.3.00 H₂O: C, 41.79; H, 5.49; N, 11.25; S, 8.58. Found: C, 41.79; H, 5.54; N, 11.16; S, 8.37.

### Example H21

### (4-Amino-2-{1-[6-(2-cyclopropylamino-ethoxy)-3-sulfonyl]-piperidin-4-ylamino}-thiazol-5-yl)-(2,6-difluoro-phenyl)-methanone Hydrochloride Salt.

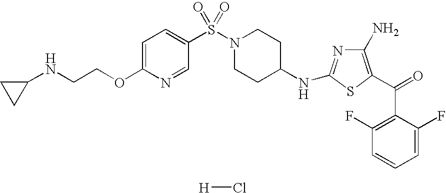

The title compound was prepared in a manner analogous to that for Example H19 from (4-{4-[4-amino-5-(2,6-difluoro-benzoyl)thiazol-2-ylamino]-piperidine-1-sulfonyl}-phenoxy)-acetaldehyde (from Example H19) and cyclopropylamine in 22% yield.

¹H NMR (DMSO-d₆): δ 8.85 (bs, 1H), 8.57-8.48 (m, 2H), 8.10-7.90 (m, 3H), 7.52-7.40 (m, 1H), 7.19-7.02 (m, 3H), 4.65-4.55 (9m, 2H), 3.48-3.35 (m, 4H), 2.80-2.70 (m, 1H), 2.09-2.05 (m, 2H), 1.98-1.85 (m, 2H), 1.58-1.40 (m, 2H), 0.9-0.72 (m, 4H), 0.66-0.58 (m, 2H). HRMALDIMS: C₂₅H₂₉F₂N₆O₄S₂ (MH⁺): 579.1660. Found: 579.1669.

### Example H22

### (4-Amino-2-{1-[2-(2-morpholin4-yl-ethoxy)-pyrimidine-5-sulfonyl]-piperidin-4-ylamino}-thiazol-5-yl)-(2,6-difluoro-phenyl)-methanone Trifluoroacetic Acid Salt.

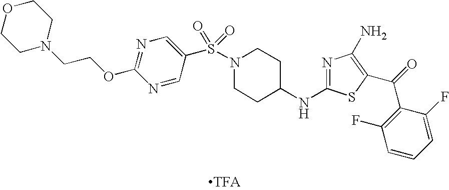

The title compound was prepared in a manner similar to that used to prepare Example H2 from {4-amino-2-[1-(2-chloro-pyrimidine-5-sulfonyl)-piperidin-4-ylamino]-thiazol-5-yl}-2,6-difluoro-phenyl)-methanone (Example F47) and 4-(2-hydroxyethyl)-morpholine.

¹H NMR (CD₃OD): δ 8.99 (s, 2H), 7.45 (m, 1H), 7.07-6.98 (m, 2H), 4.12-3.81 (m, 8H), 3.87-3.68 (m, 7H), 2.70 (m, 2H), 2.12 (m, 2H), 1.67 (m, 2H). ESIMS (MH⁺): 610. Anal. Calcd for C₂₅H₂₉F₂N₇O₅S₂.1.5TFA.0.75 H₂O: C, 42.34; H, 4.06; N, 12.35; S, 8.07. Found: C, 42.51; H, 4.05; N, 12.28; S, 8.18.

### Example H23

### (4-Amino-2-{1-[2-(2-piperidin-1-yl-ethoxy)-pyrimidine-5-sulfonyl]-piperidin-4-ylamino}-thiazol-5-yl)-(2,6-difluoro-phenyl)-methanone Trifluoroacetic Acid Salt.

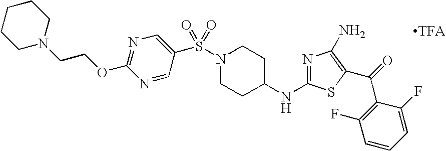

The title compound was prepared in a manner similar to that used to prepare example H2 from {4-amino-2-[1-(2-chloro-pyrimidine-5-sulfonyl)-piperidin-4-ylamino]-thiazol-5-yl}-(2,6-difluoro-phenyl)-methanone (Example F47) and 1-piperidineethanol.

¹H NMR (CD₃OD): δ 8.99 (s, 2H), 7.34 (m, 1H), 7.08-6.93 (m, 2H), 3.79-3.60 (m, 7H), 3.06 (m, 2H), 2.67 (m, 2H), 2.17-1.52 (m, 12H). ESIMS (MH⁺): 608. Anal. Calcd for C₂₆H₃₁F₂N₇O₄S₂.1.9TFA.0.75H₂O: C, 42.72; H, 4.14; N, 11.70; S, 7.65. Found: C, 42.78; H, 4.24; N, 11.87; S, 7.65.

### Example H24

### (4-Amino-2-{1-[2-(2-dimethylamino-ethoxy)-pyrimidine-5-sulfonyl]-piperidin-4-ylamino}-thiazol-5-yl)-(2,6-difluoro-phenyl)-methanone Trifluoroacetic Acid Salt.

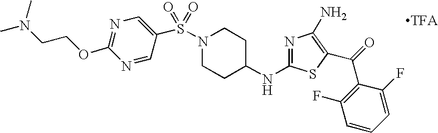

The title compound was prepared in a manner similar to that used to prepare example H2 from {4-amino-2-[1-(2-chloro-pyrimidine-5-sulfonyl)-piperidin-4-ylamino]-thiazol-5-yl}-2,6-difluoro-phenyl)-methanone (Example F47) and 2-dimethylamino-ethanol.

¹H NMR (CD₃OD): δ 8.98 (s, 2H), 7.44 (m, 1H), 7.08-6.99 (m, 2H), 3.76-3.67 (m, 3H), 3.56-3.45 (m, 2H), 3.02 (s, 6H), 2.70 (m, 2H), 2.12 (m, 2H), 1.65 (m, 2H). ESIMS (MH⁺): 568. Anal. Calcd for C₂₃H₂₇F₂N₇O₄S₂.1.5 TFA.0.70 H₂O: C, 41.56; H, 4.01; N, 13.05; S, 8.54. Found: C, 41.78; H, 4.30; N, 13.23; S, 8.61.

### Example H25

### (4-Amino-2-{1-[2-(2-dimethylamino-ethoxy)-1-methyl-1H-imidazole-4-sulfonyl]-piperidin-4-ylamino}-thiazol-5-yl)-(2,6-difluoro-phenyl)-methanone Hydrochloride Salt.

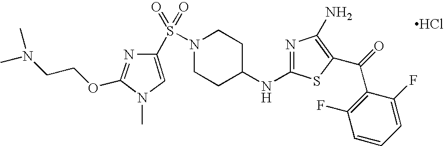

The title compound was prepared in a manner similar to that used to prepare Example H2 from {4-amino-2-[1-(2-bromo-1-methyl-1H-imidazole-4-sulfonyl)-piperidin-4-ylamino]-thiazol-5-yl}-(2,6-difluoro-phenyl)-methanone (Example F48) and 2-dimethylaminoethanol.

¹H NMR (CD₃OD): δ 7.70 (s, 1H), 7.55 (m, 1H), 7.15-7.08 (m, 2H), 4.57 (m, 2H), 3.78-3.70 (m, 3H), 3.64 (s, 6H), 3.03 (s, 3H), 2.97-2.82 (m, 4H), 2.08 (m, 2H), 1.63 (m, 2H). ESIMS (MH⁺): 570. Anal. Calcd for C₂₃H₂₉F₂N₇O₄S₂.2.40 HCl.2.00 H₂O.0.1EtOAc: C, 40.03; H, 5.20; N, 13.97; S, 9.14. Found: C, 40.21; H, 5.02; N, 13.69; S, 9.39.

### Example H26

### (4-Amino-2-{1-[6-(1-methyl-piperidin-3R-yloxy)-pyridine-3-sulfonyl]-piperidin-4-ylamino}-thiazol-5-yl)-(2,6-difluoro-phenyl)-methanone Hydrochloride

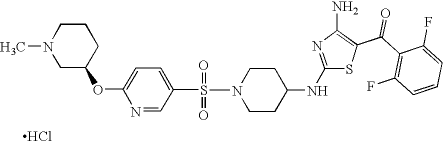

The starting materials were prepared as follows:
3R-Hydroxy-piperidine-1-carboxylic Acid tert-Butyl Ester

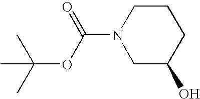

Prepared with conditions similar to that described for the racemate in de Costa, et al., J. Med. Chem., 35, 4334-4343 (1992): to a mixture of 3R-hydroxypiperidine (13.7 g, 100 mmol) and NaHCO₃ (42.0 g, 500 mmol) in water (200 ml) was added di-tert-butyl dicarbonate (26.2 g, 120 mmol). After 48 hours at ambient temperature, the resultant mixture was extracted with CH₂Cl₂ (3×100 ml). The combined organic extracts were washed with water (20 ml), dried over Na₂SO₄, and evaporated to afford 21.7 g of a colorless oil in 34% yield, which displayed an ¹H NMR spectrum that matched literature (de Costa, et al., J. Med. Chem., 35, 4334-4343 (1992)) and was used without further purification.
1-Methyl-piperidin-3R-ol

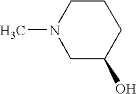

Prepared in a similar manner to that for (1-methyl-piperidin-3S-yl)-methanol in Example H11: to a solution of 3R-hydroxy-piperidine-1-carboxylic acid tert-butyl ester (10.1 g, 50.0 mmol) in THF (200 ml) at 0° C. was added LiAlH₄ (250 ml of 1M in ether, 250 mmol). The resultant mixture was heated at reflux for 24 hours, cooled to 0° C. and carefully treated with Na₂SO₄.10 H₂O until gas evolution ceased. The suspension was suction-filtered through a Büchner funnel and then gravity-filtered to afford 3.93 g of a colorless oil in 34% yield, which displayed an ¹H NMR that matched literature (for the racemate; de Costa, et al., J. Med. Chem., 35, 4334-4343 (1992)) and was used without any further purification.

The title compound was prepared in a manner like that described for Example H2. {4-Amino-2-[1-(6-chloro-pyridine-3-sulfonyl)-piperidin-4-ylaminol-thiazol-5-yl]-(2,6-difluoro-phenyl)-methanone (from Example F12) and 1-methyl-piperidin-3R-ol gave a pale white powder in 23% yield.

¹H NMR spectrum matched that of the racemate, Example H16. HRMALDIMS: Calcd. for C₂₆H₃₁N₆O₄S₂F₂ (MH⁺): 593.1816. Found: 599.1093. Anal. Calcd. For C₂₆H₃₀N₆O₄S₂F₂.1.60 HCl.1.50 H₂O: C, 46.05; H, 5.14; N, 12.39; S, 9.46. Found: C, 46.06; H, 5.14; N, 12.32; S, 9.35.

### Example H27

### (4-Amino-2-{1-[6-(1-methyl-pyrrolidin-3R-yloxy)-pyridine-3-sulfonyl]-piperidin-4-ylamino}-thiazol-5-yl)-(2,6-difluoro-phenyl)-methanone Hydrochloride

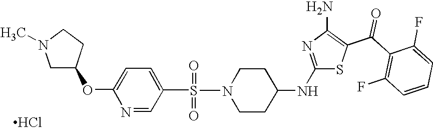

The starting material was prepared as follows:

3R-Hydroxy-pyrrolidine-1-carboxylic Acid tert-Butyl Ester

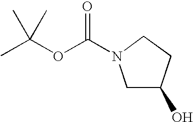

Prepared in a manner similar to that described for 3R-hydroxy-piperidine-1-carboxylic acid tert-butyl ester in Example H26, confirmed with an ¹H NMR spectrum that matched literature (Sternfeld, et al J. Med. Chem. 42, 677-690 (1999)), and used without any further purification.
1-Methyl-pyrrolidin-3R-ol.

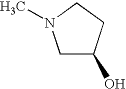

The title compound was prepared with a sequence similar to that described for 1-methyl-piperidin-3R-ol in Example H26. 3R-Hydroxy-pyrrolidine-1-carboxylic acid tert-butyl ester gave a colorless oil in 75% yield, which was used without further purification.

¹H NMR (CDCl₃): δ 4.40-4.30 (m, 1H), 2.96-2.81 (m, 2H), 2.68 (d, 1H, J=10.1 Hz), 2.52-2.42 (m, 1H), 2.36 (s, 3H), 2.28-2.18 (m, 2H), 1.78-1.67 (m, 1H).

The title compound was prepared in a manner similar to that described for Example H2. {4-Amino-2-[1-(6-chloro-pyddine-3-sulfonyl)-piperidin-4-ylamino]-thiazol-5-yl}-(2,6-difluoro-phenyl)-methanone (Example F21) and 1-methyl-pyrrolidin-3R-ol gave a pale white powder in 38% yield.

¹H NMR (DMSO-d₆): δ 8.80 (bs, 1H), 8.55 (s, 1H), 8.09-7.90 (m, 3H), 7.56-7.41 (m, 1H), 7.15 (t, 2H, J=7.9 Hz), 7.08-7.02 (m, 1H), 5.70-5.56 (m, 1H), 3.56-3.42 (m, 3H), 3.30-3.11 (m, 2H), 2.94-2.85 (m, 4H), 2.69-2.53 (m, 3H), 2.31-2.11 (m, 2H), 1.98-1.85 (m, 2H), 1.63-1.45 (m, 2H). HRMALDIMS: Calcd. for C₂₅H₂₉N₆O₄S₂F₂ (MH⁺): 579.1660. Found: 579.1652. Anal. Calcd. For C₂₅H₂₈N₆O₄S₂F₂.1.85 HCl.1.00 H₂O: C, 45.21; H, 4.83; N, 12.65; S, 9.66. Found: C, 45.23; H, 5.08; N, 12.49; S, 9.51.

### Example H28

### (4-Amino-2-{1-[6-(1-methyl-pyrrolidin-3S-yloxy)-pyridine-3-sulfonyl]-piperidin-4-ylamino}-thiazol-5-yl)-(2,6-difluoro-phenyl)-methanone Hydrochloride

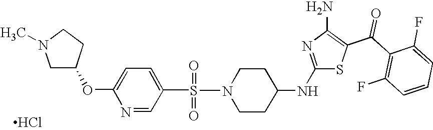

The starting material, 1-methyl-pyrrolidin-3S-ol, was prepared with a sequence identical to that for 1-methyl-pyrrolidin-3R-ol from Example H27. 3S-Hydroxy-pyrrolidine-1-carboxylic acid tert-butyl ester gave a colorless oil in 85% yield, which was used without further purification.

¹H NMR spectrum was identical to that for 1-methyl-pyrrolidin-3R-ol from Example H27.

The title compound was prepared in a manner like that described Example H2. {4-Amino-2-[1-(6-chloro-pyridine-3-sulfonyl)-piperidin-4-ylamino]-thiazol-5-yl)(2,6-difluoro-phenyl)-methanone (from Example F21) and 1-methyl-pyrrolidin-3S-ol gave a pale white powder in 36% yield.

¹H NMR spectrum was identical to that for Example H27. HRMALDIMS: Calcd. for C₂₅H₂₉N₆O₄S₂F₂ (MH⁺): 579.1660. Found: 579.1653. Anal. Calcd. For C₂₅H₂₈N₆O₄S₂F₂.2.20 HCl 3.00 H₂O: C, 42.12; H, 5.12; N, 11.79; S, 9.00. Found: C, 45.29; H, 5.12; N, 11.74; S, 8.87.

### Example H29

### (4-Amino-2-{1-[6-(1-methyl-piperidin-3S-yloxy)-pyridine-3-sulfonyl]-piperidin-4-ylamino}-thiazol-5-yl)-(2,6-difluoro-phenyl)-methanone Hydrochloride

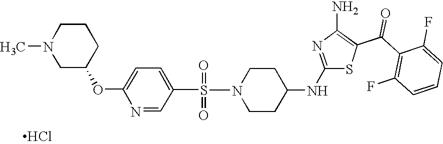

The title compound was prepared in a manner like that described in Example H2. {4-Amino-2-[1-(6-chloro-pyridine-3-sulfonyl)-piperidin-4-ylamino]-thiazol-5-yl}-(2,6-difluoro-phenyl)-methanone (Example F21) and 1-methyl-piperidin-3S-ol (using a procedure reported for the R isomer from Cossy, et al., Eur. J. Org. Chem., 1693-1699 (1999) afforded a pale white powder in 38% yield.

¹H NMR spectrum was identical to that of the racemate, Example H16. HRMALDIMS: Calcd. for C₂₆H₃₁N₆O₄S₂F₂ (MH⁺): 593.1816. Found: 599.1093. Anal. Calcd. For C₂₆H₃₀N₆O₄S₂F₂.1.90 HCl 0.20 EtOAc.0.80 H₂O: C, 46.38; H, 5.10; N, 12.11; S, 9.24. Found: C, 46.28; H, 5.33; N, 12.11; S, 9.04.
Method I:

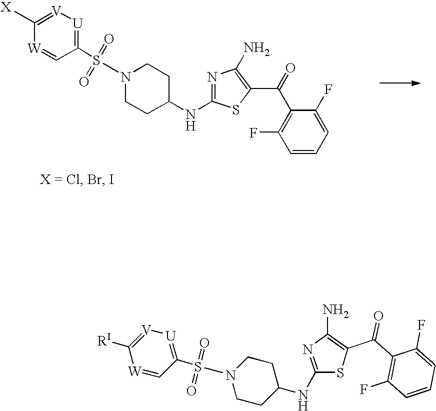

### Example I1

### 1-(4-Amino-2-{1-[6-(1H-imidazol-2-yl)-pyridine-3-sulfonyl]-piperidin-4-ylamino}-thiazol-5-yl)-1-(2,6-difluoro-phenyl)-methanone Hydrochloride.

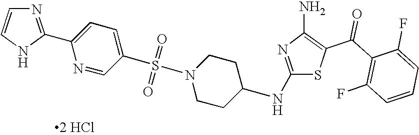

The starting material was prepared as follows:
1-Methoxymethyl-imidazole

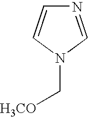

To a solution of imidazole (1.00 g, 14.7 mmol) in anhydrous THF (30 ml) at −78° C. was added in portions sodium hydride (0.88 9 of a 60% dispersion in oil, 22.0 mmol). The mixture was allowed to warm to room temperature, stirred for 30 minutes, then cooled to −78° C., and chloromethyl methyl ether (1.06 ml, 14.0 mmol) slowly added. After 2 hours at −78° C., sat. NaHCO₃ was added to quench the reaction. The solvent was removed and a solution of the resultant residue in ethyl acetate was washed with sat. NaHCO₃, dried over MgSO₄, filtered, and concentrated to give 1.3 g of an oil, which contained the NaH dispersion oil, displayed an ¹H NMR that matched previous (Zhao, et al., J. Med. Chem., Vol. 40, pp. 216-225 (1997)), and was used without further purification.

The title compound was prepared as follows. To a solution of 1-methoxymethyl-imidazole (216 mg, 1.95 mmol) in dry THF (20 ml) at −78° C. was added slowly a solution of t-butyllithium (2.4 ml of 1.7 M in THF). After 20 minutes, ZnCl₂ (663 mg, 4.86 mmol) was added, the mixture was allowed to warm to room temperature and stirred for another 60 min. 1-{4-Amino-2-[1-(6-chloro-pyridine-3-sulfonyl)-piperidin-4-ylamino]-thiazol-5-yl}-(1-phenyl-methanone (Example F21; 200 mg, 0.390 mmol) and tetrakis(triphenylphosphino)palladium(0) (Pd(Ph₃P)₄; 12 mg, 0.013 mmol) were added and the mixture refluxed under argon for 2 hours. The solvent was removed and a solution of the resultant residue in ethyl acetate was washed with 0.1 NaOH, dried over MgSO₄, filtered, and concentrated. The resultant solid was dissolved in a solution of 38% HCl (10 ml), ethanol (15 ml), and H₂O (15 ml) and refluxed for 2 hours. The solvent was removed and a solution of the resultant residue in ethyl acetate was washed with sat. NaHCO₃, dried over MgSO₄, filtered, concentrated, and purified via preparative HPLC. The concentrate from fractions was dissolved in EtOAc, washed with sat NaHCO₃, dried over MgSO₄, filtered, and concentrated. The resultant solid was placed in acetonitrile (30 ml), water (90 ml), and 38% HCl (0.5 mL) and evaporated to give 26 mg of white powder in 11% yield.

¹H NMR (CD₃OD): δ 9.13 (d1H, J=2.5 Hz), 8.44 (dd, 1H, J=2.5, 8.3 Hz), 8.23 (d, 1H, J=8.3 Hz), 7.78 (s, 2H), 7.50-7.40 (m, 1H), 7.08-6.97 (m, 2H), 4.02-3.90 (m, 3H), 2.98-2.87 (m, 2H), 2.37-2.13 (m, 2H), 1.96-1.78 (m, 2H). ESIMS (MH⁺): 546. Anal. Calcd for C₂₃H₂₁F₂N₇O₃S₂.2.4 HCl.1.0 H₂O.0.5 EtOAc: C, 43.19; H, 4.26; N, 14.10; S, 9.23. Found: C, 42.85; H, 4.67; N, 14.50; S, 9.27.

### Example I2

### 1-(4-Amino-2-{1-[6-(4-methyl-1H-imidazol-2-yl)-pyridine-3-sulfonyl]-piperidin-4-ylamino}-thiazol-5-yl)-1-(2,6-difluoro-phenyl)-methanone Hydrochloride.

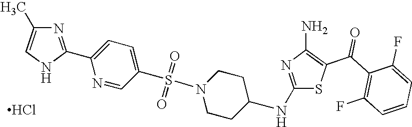

The title compound was prepared through a route with conditions similar to that for Example I1. 4-Methylimidazole and 1-{4-amino-2-[1-(6-chloro-pyridine-3-sulfonyl)-piperidin-4-ylamino]-thiazol-5-yl}-1-phenyl-methanone (Example F21), preparative HPLC purification and treatment of the fractions with HCl prior to lyophilization gave a white solid in 30% overall yield.

¹H NMR (CD₃OD): δ 9.12 (d1H, J=2.3 Hz), 8.47 (dd, 1H, J=2.3, 8.3 Hz), 8.23 (d, 1H, J=8.3 Hz), 7.53-7.42 (m, 2H), 7.10-6.98 (m, 2H), 3.82-3.74 (m, 3H), 2.80-2.69 (m, 2H), 2.48 (s, 3H), 2.16-2.07 (m, 2H), 1.72-1.59 (m, 2H). HRMALDIMS. Calcd for C₂₄H₂₄F₂N₇O₃S₂ (MH⁺): 560.1345. Found: 560.1338. Anal. Calcd for C₂₄H₂₃F₂N₇O₃S₂.2.0 HCl.1.0 H₂O: C, 44.71; H, 4.38; N, 14.48; S, 9.47. Found: C, 44.31; H, 4.28; N, 14.25; S, 9.92.

### Example I3

### 1-(4-Amino-2-{1-[6-(1-methyl-1H-imidazol-2-yl)-pyridine-3-sulfonyl]-piperidin-4-ylamino}-thiazol-5-yl)-1-(2,6-difluoro-phenyl)-methanone Trifluoroacetic Acid Salt.

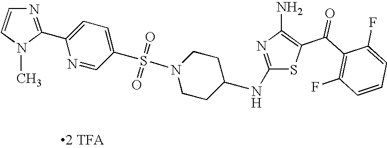

The title compound was prepared in a manner similar to that for Example I1. 1-Methyl-imidazole was processed, coupled with 1-{4-amino-2-[1-(6-chloro-pyridine-3-sulfonyl)-methanone (Example F21), and purified via preparative HPLC.

¹H NMR (CD₃OD): δ 9.13 (s, 1H), 8.46-8.38 (m, 1H), 8.20 (d, 1H, J=8.3 Hz), 7.75-7.67 (m, 2H), 7.46-7.32 (m, 2H), 7.01-6.92 (m, 2H), 4.22 (s, 3H), 3.70-3.59 (m, 3H), 2.75-2.63 (m, 2H), 2.12-2.02 (m, 2H), 1.69-1.54 (m, 2H). ESIMS (MH⁺): 560. Anal. Calcd for C₂₄H₂₃F₂N₇O₃S₂.2.0 TFA: C, 42.69; H, 3.20; N, 12.45; S, 8.14. Found: C, 42.49; H, 3.46; N, 12.43; S, 8.11.

### Example I4

### 1-(4-Amino-2-{1-[6-(1H-imidazol-2-ylmethyl)-pyridine-3-sulfonyl]-piperidin-4-ylamino}-thiazol-5-yl)-1-(2,6-difluoro-phenyl)-methanone Hydrochloride.

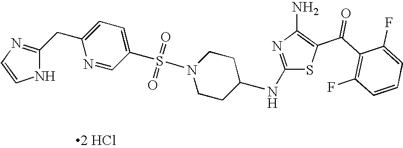

The starting materials were prepared as follows:
2-Methyl-1-triphenylmethyl-imidazole

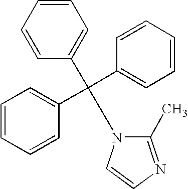

A mixture of 2-methyl-imidazole (0.82 g, 10 mmol), triphenylmethyl chloride (2.78 g, 10.0 mmol), and triethylamine (1.0 g, 10 mmol) in DMF (10 ml) stirred at room temperature for 2 hours. The DMF was removed under reduced pressure. The resultant residue was dissolved in ethyl acetate, washed with 0.1 N NaOH, dried over MgSO₄, filtered, and concentrated. The resultant solid was triturated with ethyl ether, collected by filtration, and dried under vacuum to give 3.0 g of white solid in 95% yield, which displayed a ¹H NMR spectrum that matched previous (Kirk, J. Org. Chem., Vol. 43, pp. 4381-4383 (1978)) and was used without further purification.
1-(4-Amino-2-{4-[6-(1-triphenylmethyl-1H-imidazol-2-ylmethyl)-pyridine-3-sulfonyl]-cyclohexylamino}-thiazol-5yl)-1-(2,6-difluoro-phenyl)-methanone

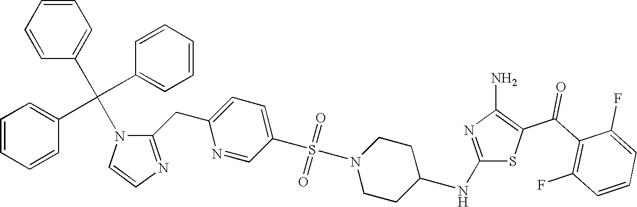

Prepared in a manner similar to that for Example I1. 2-Methyl-1-triphenylmethyl-imidazole was processed and coupled with 1-{4-amino-2-[1-(6-chloro-pyridine-3-sulfonyl)-piperidin-4-ylamino]-thiazol-5-yl}-1-phenyl-methanone (Example F21) and used without further purification.

¹H NMR (CD₃OD): δ 8.80 (d, 1H, J=2.0 Hz), 8.12 (dd, 1H, J=2.0, 8.2 Hz), 7.62 (d, 1H J=8.2 Hz), 7.50-7.15 (m, 18H), 7.12-7.06 (m, 2H), 4.60, (s, 2H), 3.85 (br, 1H), 3.68-3.60 (m, 2H), 2.66-2.58 (m, 2H), 2.08-2.00 (m, 2H), 1.66-1.58 (m, 2H).

The title compound of this Example was prepared as follows. 1-(4-Amino-2-{4-[6-(1-triphenylmethyl-1H-imidazol-2-ylmethyl)-pyridine-3-sulfonyl]-cyclohexylamino}-thiazol-5-yl)-1-(2,6-difluoro-phenyl)-methanone was dissolved in 10% TFA/CH₂Cl₂ and stirred at room temperature for 30 min. The solvent was removed in vacuo and the crude was purified via preparative HPLC to give 53 mg of white powder in 47% yield (over two steps, from 2-chloropyridine and Example F21).

¹H NMR (CD₃OD): δ 8.80 (d, 1H, J=2.0 Hz), 8.12 (dd, 1H, J=2.0, 8.2 Hz), 7.62 (d, 1H J=8.2 Hz), 7.50 (m, 1H), 7.42 (s, 2H), 7.10-7.06 (m, 2H), 4.60, (s, 2H), 3.85 (br, 1H), 3.66-3.60 (m, 2H), 2.64-2.58 (m, 2H), 2.06-2.00 (m, 2H), 1.66-1.58 (m, 2H). LCESIMS (MH⁺): 560. Anal. Calcd for C₂₄H₂₃F₂N₇O₃S₂.2.5 HCl.1.0 H₂O: C, 43.10; H, 4.14; N, 14.66; S, 9.59. Found: C, 43.25; H, 4.40; N, 14.69; S, 9.39.

### Example I5

### 1-[4-Amino-2-{1-[6-(1-methyl-1H-imidazol-4-yl)-pyridine-3-sulfonyl]-piperidin-4-ylamino}-thiazol-5-yl)-1-(2,6-dihydroxy-phenyl)-methanone Hydrochloride.

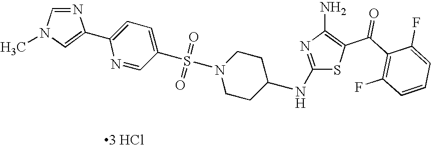

The title compound was prepared as follows. A mixture of 4-iodo-1-methyl-imidazole (207 mg, 1.00 mmol; Combi-Blocks, Inc.), bis(pinacolato)-diboron (279 mg, 1.10 mmol), potassium acetate (294 mg, 3.00 mmol), and 1,1′-bis(diphenylphosphino)-ferrocene dichloropalladium(II) (PdCl₂(dppf); 24 mg, 0.03 mmol) in DMF (10 ml) was heated at 80° C. for 2 hours. The mixture was allowed to cool to room temperature and 1-{4-amino-2-[1-(6-chloro-pyridine-3-sulfonyl)-piperidin-4-ylamino]-thiazol-5-yl}-1-phenyl-methanone (Example F21; 180 mg, 0.500 mmol), 2M Na₂CO₃ (0.5 ml), and additional PdCl₂(dppf) (24 mg, 0.03 mmol) were added sequentially. The mixture was heated at 80° C. overnight. The solvent was removed under reduced pressure and a solution of the resultant residue in ethyl acetate was washed with 0.1N NaOH and brine, dried over MgSO₄, filtered, and concentrated to a crude solid, which was purified via preparative HPLC and fractions treated with HCl prior to lyophilization to give 14 mg of white powder in 5% yield.

¹H NMR (CD₃OD): δ 9.04 (s, 2H), 9.00 (s, 1H), 8.34-8.29 (m, 2H), 8.08 (d, 1H, J=8.1 Hz), 7.60-7.48 (m, 1H,), 7.02 (m, 2H), 4.04 (s, 3H), 3.78-3.73 (m, 2H), 2.73-2.69 (m, 2H), 2.14-2.10 (m, 2H), 1.68-1.62 (m, 2H). HRMALDIMS.: C₂₄H₂₄F₂N₇O₃S₂ (MH⁺): 560.1345. Found: 560.1360. Anal. Calcd. For C₂₄H₂₃F₂N₇O₃S₂.0.58 EtOAc.2.84 HCl: C, 44.26; H, 4.30; N, 13.73; S, 8.98. Found: C, 44.25; H, 4.49; N, 13.73; S, 8.81.

### Example I6

### 1-{4-Amino-2-[1-([2,3′]bipyridinyl-5-sulfonyl)-piperidin-4-ylamino]-thiazol-5-yl}-1-(2,6-difluoro-phenyl)-methanone Hydrochloride.

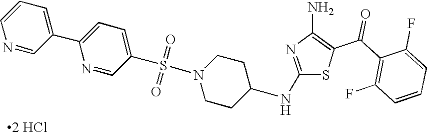

The title compound was prepared as follows. A solution of 1-{4-amino-2-[1-(6-chloro-pyridine-3-sulfonyl)-piperidin-4-ylamino]-thiazol-5-yl}-1-phenyl-methanone (Example F21; 1.50 g, 2.92 mmol), diethyl(3-pyridyl)borane (4.30 g, 29.2 mmol), Pd(Ph₃P)₄ (0.70 g, 0.61 mmol), and K₂CO₃ (6.0 g) in H₂O/THF (30/80 ml) was degassed and heated at reflux for 72 hours. The mixture was allowed to cool to room temperature and diluted with ethyl acetate. The resultant organic solution was washed with sat. NaHCO₃ (3×150 ml), dried over MgSO₄, filtered, and concentrated. Column chromatography with 5% MeOH/EtOAc provided 0.94 g of yellow solid in 58% yield, which was placed in 30% CH₃CN/H₂O, treated with excess 1N HCl, and lyophilized.

¹H NMR (CD₃OD): δ 9.63 (s, 1H), 9.36 (d, 1H, J=8.1 Hz), 9.11 (s, 1H), 8.97 (d, 1H, J=5.3 Hz), 8.39 (s, 2H), 8.30-8.22 (m, 1H), 7.58-7.47 (m, 1H), 7.13-7.04 (m, 2H), 3.83-3.72 (m, 3H), 2.79-2.68 (m, 2H), 2.17-2.03 (m, 2H), 1.73-1.60 (m, 2H). ESIMS (MH⁺): 557. Anal. Calcd for C₂₅H₂₂F₂N₆O₃S₂.2.5 HCl.0.75 H₂O: C, 45.41; H, 3.96; N, 12.71; S, 9.70. Found: C, 45.67; H, 4.26; N, 12.61; S, 9.55.

### Example I7

### 1-{4-Amino-2-[1-([2,4′]bipyridinyl-5-sulfonyl)-piperidin in-4-ylamino]-thiazol-5-yl}-1-(2,6-difluoro-phenyl)-methanone.

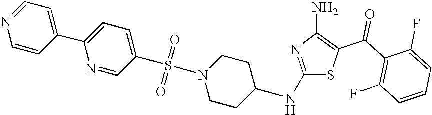

The title compound was prepared in a manner similar to that for 1-{4-amino-2-[1-([2,3′]bipyridinyl-5-sulfonyl)-piperidin-4-ylamino]-thiazol1-5-yl}-1-(2,6-difluoro-phenyl)-methanone (Example I6). 1-{4-Amino-2-[1-(6-chloro-pyridine-3-sulfonyl)-piperidin-4-ylamino]-thiazol-5-yl}-1-phenyl-methanone (Example F21; 410 mg, 0.789 mmol) and 4-pyridylboronic acid (490 mg, 0.398 mmol; Frontier Scientific, Inc.) and purification via column chromatography with 0.5% (58% NH₄OH)/5%MeOH/CH₂Cl₂ as eluant gave a yellow solid in 11% yield.

¹H NMR (CD₃OD): δ 8.92 (d, 1H, J=2.0 Hz), 8.70 (d, 2H, J=8.0 Hz), 8.38 (dd, 1H, J=2.4, 8.7 Hz), 7.88 (d, 1H, J=8.7 Hz), 7.48-7.38 (m, 1H), 7.00 (dd, 2H, J=7.5, 8.3 Hz), 6.58 (d, 2H, J=8.0 Hz), 2.72 (dd, 2H, J=10.2, 10.3 Hz), 1.72-1.68 (m, 2H). Anal. Calcd. for C₂₅H₂₂F₂N₆O₃S₂.1.8 H₂O.0.2 MeOH: C, 50.83; H, 4.47; N, 14.11; S, 10.77. Found: C, 50.99; H, 4.14; N, 13.92; S, 10.41.

### Example I8

### 1-{4-Amino-2-[1-(4-pyridin-3-yl-benzenesulfonyl)-piperidin-4-ylamino]-thiazol-5-yl}-1-(2,6-difluoro-phenyl)-methanone Trifluoroacetic Acid Salt.

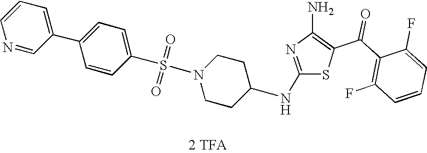

The title compound was prepared as follows. According to conditions from Bleicher, et al, J. Org. Chem., Vol. 43, pp. 1109-1118 (1998), to a mixture of 1-{4-amino-2-[1-(4-iodo-benzenesulfonyl)-piperidin-4-ylamino]-thiazol-5-yl}-1-(2,6-difluoro-phenyl)-methanone (Example F42; 600 mg, 1.00 mmol) and K₂CO₃ (0.22 g, 2.5 mmol) in DME (3.6 ml) and H₂O (1.6 ml) were added sequentially Pd/C (10% wt, 27 mg), CuI (9.5 mg) and PPh₃ (25 mg). The mixture stirred for a half hour and diethyl (3-pyridyl)borane (0.37 g, 2.5 mmol) was added. After heating at 80° C. for 4 hours, additional Pd/C, CuI, PPh₃, and more diethyl(3-pyridyl)borane (1.03 g, 6.95 mmol) were added. After 3 days at 80° C., methanol was added and the mixture was filtered. The filtrate was concentrated and ethyl acetate added. The organic solution was washed with water, separated, dried over MgSO₄, filtered, and concentrated to give a yellow solid, which was purified via preparative HPLC to afford 0.26 g of yellow solid in 47% yield.

¹H NMR (DMSO-d₆): δ 8.99 (s, 1H), 8.65 (d, 1H, J=4.9 Hz), 8.27 (dt, 1H, J=1.6, 8.8 Hz), 7.96 (d, 2H, J=8.5 Hz), 7.91 (br, 2H), 7.76 (d, 2H, J=8.5 Hz), 7.62 (dd, 1H, J=4.9, 7.9 Hz), 7.39 (m, 1H), 7.05 (dd, 2H, J=7.6, 8.2 Hz), 3.42-3.39 (m, 2H), 2.58-2.45 (m, 2H), 1.93-1.79 (m, 2H), 1.54-1.38 (m, 2H). LC-ESIMS: (MH⁺): 556. Anal. Calcd. for C₂₆H₂₃F₂N₅O₃S₂.2.0 TFA.0.5 H₂O: C, 45.46; H, 3.31; N, 8.83; S, 8.09. Found: C, 45.54; H, 3.54; N, 8.65; S, 8.00.

### Example I9

### 1-(4-Amino-2-{1-[4-(3-dimethylamino-prop-1-ynyl)-benzenesulfonyl]-piperidin-4-ylamino}-thiazol-5-yl)-1-(2,6-difluoro-phenyl)-methanone D-Glucuronic Acid Salt.

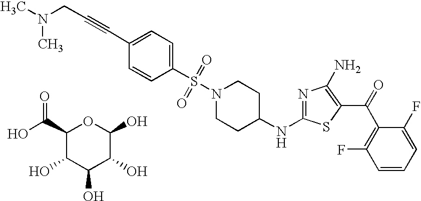

Starting material was made as follows.

1-(4-Amino-2-{1-[4-(3-dimethylamino-prop-1-ynyl)-benzenesulfonyl]-piperidin-4-ylamino}-thiazol-5-yl)-1-(2,6-difluoro-phenyl)-methanone

Prepared in a manner similar to that for 1-{4-amino-2-[1-(4-pyridin-3-yl-benzenesulfonyl)-piperidin-4-ylamino]-thiazol-5-yl}-1-(2,6-difluoro-phenyl)-methanone trifluoroacetic acid salt (Example I8) and consistent with a procedure given in Bleicher, et al., J Org. Chem., Vol. 63, pp. 1109-1118 (1998). 1-{4-Amino-2-[1-(4-iodo-benzenesulfonyl)-piperidin-4-ylamino]-thiazol-5-yl}-1-(2,6-difluoro-phenyl)-methanone (Example F42) and 1-dimethylamino-2-propyne coupled to give a dark brown solid, which recrystallized from ethyl acetate to obtain 250 mg of light brown crystals in 58% yield.

¹H NMR (DMSO-d₆): δ 8.00 (br, 2H), 7.72 (d, 2H, J=8.7 Hz), 7.67 (d, 2H, J=8.7 Hz), 7.48 (m, 1H), 7.14 (dd, 2H, J=7.6, 8.1 Hz), 3.50 (s, 2H), 2.26 (s, 6H), 1.92-1.83 (m, 2H), 1.58-1.40 (m, 2H). LC-ESIMS(MH⁺): 560. Anal. Calcd. for C₂₆H₂₇F₂N₅O₃S₂.0.35 H₂O: C, 55.18; H, 4.93; N, 12.37; S, 11.33. Found: C, 55.15; H, 4.98; N, 12.34; S, 11.18.

The title compound was prepared as follows. 1-(4-Amino-2-{1-[4-(3-dimethylamino-prop-1-ynyl)-benzenesulfonyl]-piperidin-4-ylamino}-thiazol-5-yl)-1-(2,6-difluoro-phenyl)-methanone (100 mg, 0.179 mmol) and D-glucuronic acid (35 mg, 0.18 mmol) were placed in 95% ethanol (5 ml), heated to boiling, and water added until a clear solution was obtained. The solvent was removed in vacuo. A solution of the resultant white solid in hot ethanol was diluted with water until a white precipitate was obtained. Filtration and drying led to 104 mg of yellow solid in 69% yield, mp determination attempt accompanied by foaming and decomposed above 100° C.

¹H NMR (D₂O): δ 7.53 (bs, 4H), 7.20 (bt, 1H, J=6.9 Hz), 6.74 (bt, 2H, J=7.3 Hz), 5.18 (d, 1H, J=3.1 Hz), 4.13 (s, 2H), 3.62-3.28 (m, 8H), 3.11 (dd, 1H, J=8.2, 8.7 Hz), 2.83 (s, 6H), 2.10-1.75 (m, 2H), 1.68-1.55 (m, 2H), 1.48-1.30 (m, 2H), 1.01 (t, 3H, J=7.1 Hz). Anal. Calcd. for C₂₆H₂₇F₂N₅O₃S₂.C₆H₁₀O₇.EtOH.2 H₂O: C, 48.85; H, 5.67; N, 8.38; S, 7.67. Found: C, 49.17; H, 5.53; N, 8.23; S, 7.58.

### Example I10

1-(4-Amino-2-{1-[4-(3-dimethylamino-propyl)-benzenesulfonyl]-piperidin-4-ylamino}-thiazol-5-yl)-1-(2,6-difluoro-phenyl)-methanone D-Glucuronic Acid Salt.

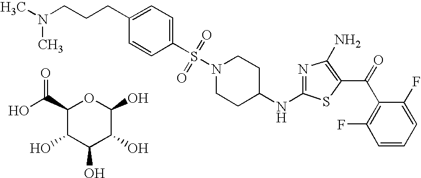

The starting material was prepared as follows.

1-(4-Amino-2-{1-[4-(3-dimethylamino-propyl)-benzenesulfonyl]-piperidin-4-ylamino}-thiazol-5-yl)-1-(2,6-difluoro-phenyl)-methanone

A mixture of 10% Pd/C (40 mg, wet DeGussa type) in acetic acid (1 ml) stirred under hydrogen atmosphere for 15 minutes prior to addition of a solution of 1-(4-amino-2-{1-[4-(3-dimethylamino-prop-1-ynyl)-benzenesulfonyl]-piperidin-4-ylamino}thiazol-5-yl)-1-(2,6-difluoro-phenyl)-methanone (from Example I9; 100 mg 0.15 mmol) in acetic acid (2 ml). After 5 hours, the catalyst was filtered off and rinsed. The filtrate was concentrated in vacuo to a yellow solid that was purified via radial chromatography with a step gradient of 0.5% (58% NH₄OH)/2% MeOH/CHCl₃ to 1% (58% NH₄OH)/10% MeOH/CHCl₃, and recrystallized from CHCl₃/hexane to afford 62 mg of desired product as a white solid in 73% yield, mp 117-120° C.

¹H NMR: δ 7.66 (d, 2H, J=8.3 Hz), 7.37 (d, 2H, J=8.3 Hz), 7.35-7.25 (m, 1H), 6.90 (ddd, 2H, J=1.1, 7.1, 8.2 Hz), 5.82 (bs, 1H), 3.68 (bd, 2H, J=12.4 Hz), 3.38 (bs, 1H), 2.72 (dd, 2H, J=7.3, 7.3 Hz), 2.48 (ddd, 2H, J=2.4, 12.1, 12.1 Hz), 2.30 (dd, 2H, J=7.3, 7.3 Hz), 2.24 (s, 6H), 2.09 (dd, 2H, J=2.9, 13.1 Hz), 1.90-1.55 (m, 6H). FTIR (KBr): 3310, 2941, 1619, 1551, 1464, 1354, 1162, 1092, 1002 cm⁻¹. ESIMS: (MH⁺) 564. Anal. Calcd. for C₂₆H₃₁F₂N₅O₃S₂.0.2 CHCl₃.0.9 H₂O: C, 52.12; H, 5.51; N, 11.60; S, 10.62. Found: C, 52.12; H, 5.40; N, 11.55; S, 10.68.

The title compound was prepared in a manner analogous to that for 1-(4-amino-2-{1-[4-(3-dimethylamino-prop-1-ynyl)-benzenesulfonyl]-piperidin-4-ylamino}-thiazol-5-yl)-1-(2,6-difluoro-phenyl)-methanone D-glucuronic acid salt (Example I9) to afford 28 mg of yellow solid in 43% yield: mp determination attempt, foaming and decomp above 125° C.

¹H NMR (CD₃OD): δ 7.74 (d, 2H, J=8.3 Hz), 7.52 (d, 2H, J=8.3 Hz), 7.44 (ddd, 1H, J=6.4, 8.4, 14.9 Hz), 7.02 (ddd, 2H, J=3.3, 7.4, 8.3 Hz), 5.15 (d, 1H, J=3.7 Hz), 4.50 (d, 1H, J=7.8 Hz), 4.11 (d, 1H, J=10.1 Hz), 3.76-3.57 (m, 11H), 3.44 (ddd, 1H, J=3.8, 3.8, 4.8 Hz), 3.41 (ddd, 1H, J=1.7, 3.4, 6.0 Hz), 3.18 (dd, 1H, J=7.9, 9.0 Hz), 2.99 (dd, 2H, J=8.0, 8.0 Hz), 2.85-2.78 (m, 8H), 2.56 (t, 2H, J=11.1 Hz), 2.08 (ddd, 4H, J=8.0, 11.8, 12.6 Hz), 1.62 (ddd, 2H, J=4.0, 11.1, 20.1 Hz), 1.20 (t, 1.5H, J=7.0 Hz). Anal. Calcd. for C₂₆H₃₁F₂N₅O₃S₂.C₆H₁₀O₇.0.5 EtOH.2 H₂O: C, 48.52; H, 5.92; N, 8.57; S, 7.85. Found: C, 48.81; H, 5.90; N, 8.35; S, 7.74.

### Example I11

### 1-(4-Amino-2-{1-[6-(3-dimethylamino-prop-1-ynyl)-pyridine-3-sulfonyl]-piperidin-4-ylamino}-thiazol-5-yl)-1-(2,6-difluoro-phenyl)-methanone.

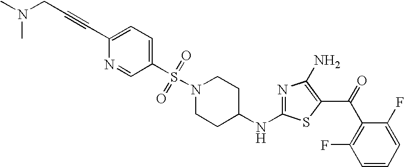

The title compound was prepared in a manner similar to that for 1-{4-amino-2-[1-(4-pyridin-3-yl-benzenesulfonyl)-piperidin-4-ylamino]-thiazol-5-yl}-1l-(2,6-difluoro-phenyl)-methanone trifluoroacetic acid salt (Example I8). 1-{4-Amino-2-[1-(6-chloro-pyridine-3-sulfonyl)-piperidin-4-ylamino]-thiazol-5-yl}-1-phenyl-methanone (Example F21) and 1-dimethylamino-2-propyne coupled to give 310 mg of white solid in 55% yield.

¹H NMR (DMSO-d₆): δ 8.85 (s, 1H), 8.12 (dd, 1H, J=2.1, 8.1, 1 Hz), 7.99 (br, 2H), 7.75 (d, 1H, J=8.1 Hz), 7.48 (m, 1H), 7.14 (dd, 2H, J=8.0, 7.7 Hz), 3.56 (s, 2H), 3.55-3.45 (m, 2H), 2.75-2.61 (m, 2H), 2.28 (s, 6H), 1.99-1.83 (m, 2H), 1.57-1.42 (m, 2H,). Anal. Calcd. for C₂₅H₂₆F₂N₆O₃S₂: C, 53.56; H, 4.67; N, 14.99; S, 11.44. Found: C, 53.30; H, 4.71; N, 14.90; S, 11.33.

### Example I12

### 1-(4-Amino-2-{1-[6-(3-dimethylamino-propyl)-pyridine-3-sulfonyl]-piperidin-4-ylamino}-thiazol-5-yl)-1-(2,6-difluoro-phenyl)-methanone.

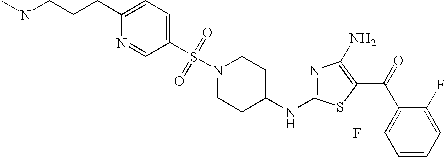

The title compound was prepared in a manner similar to that for 1-(4-amino-2-{1-[4-(3-dimethylamino-propyl)-benzenesulfonyl]-piperidin-4-ylamino}-thiazol-5-yl)-1-(2,6-difluoro-phenyl)-methanone in Example I10. 1-(4-Amino-2-{1-[6-(3-dimethylamino-prop-1-ynyl)-pyridine-3-sulfonyl]-piperidin-4-ylamino}-thiazol-5-yl)-1-(2,6-difluoro-phenyl)-methanone (Example I11) was hydrogenated and purified via preparative HPLC to give 75 mg of a hard yellow foam in 74% yield.

¹H NMR (CD₃OD): δ 8.73 (d, 1H, J=1.9 Hz), 7.98 (dd, 1H, J=2.4, 8.2 Hz), 7.44 (d, 1H, J=8.2 Hz), 7.32 (m, 1H), 6.90 (dd, 2H, J=7.4, 7.4 Hz), 3.70-3.52 (m, 3H), 2.82 (t, 2H, J=7.6 Hz), 2.54 (t, 2H, J=10.5 Hz), 2.40 (dd, 2H, J=6.2, 7.6 Hz), 2.04-1.82 (m, 4H), 1.60-1.43 (m, 2H). Anal. Calcd. for C₂₅H₃₀F₂N₆O₃S₂.0.8 H₂O: C, 51.85; H, 5.50; N, 14.51; S, 11.07. Found: C, 52.14; H, 5.48; N, 14.33; S, 10.88.

### Example I13

### 1-(4-Amino-2-{1-[6-(3-pyrrolidin-1-yl-propyl)-pyridine-3-sulfonyl]-piperidin-4-ylamino}-thiazol-5-yl)-1-(2,6-difluoro-phenyl)-methanone Trifluoroacetic Acid Salt.

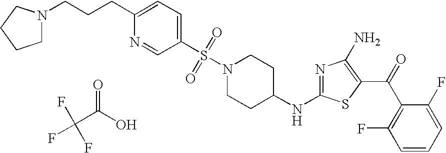

The starting material was prepared as follows.
1-(4-Amino-2-{1-[6-(3-pyrrolidin-1-yl-prop-2-ynyl)-pyridine-3-sulfonyl]-piperidin-4-ylamino}-thiazol-5-yl)-1-(2,6-difluorophenyl)-methanone

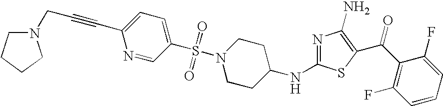

Prepared in a manner analogous to that for 1-{4-amino-2-[1-(4-pyridin-3-yl-benzenesulfonyl)-piperidin-4-ylamino]-thiazol-5-yl}-1-(2,6-difluoro-phenyl)-methanone trifluoroacetic acid salt (Example I8). 1-{4-Amino-2-[1-(4-iodo-benzenesulfonyl)-piperidin-4-ylamino]-thiazol-5-yl}-1-(2,6-difluoro-phenyl)-methanone (Example F42) and 1-prop-2-ynyl-pyrrolidine (Viola, et al., J. Org. Chem., Vol. 58, pp. 5067-75 (1993)) coupled to give 310 mg of white solid in 55% yield, which was used without any further purification.

¹H NMR (DMSO-d₆): δ 10.80 (br, 1H), 9.15 (s, 1H), 8.46 (dd, 1H, J=2.2, 8.3 Hz), 8.23 (br, 2H), 8.12 (d, 1H, J=8.3 Hz), 7.72 (m, 1H), 7.38 (dd, 2H, J=7.7, 8.1 Hz), 4.77 (s, 2H), 3.91-3.70 (m, 4H), 3.43 (br, 2H), 2.98-2.80 (m, 1H), 2.38-2.10 (m, 6H), 1.81-0.17 (m, 2H). LCESIMS (MH⁺): 587.15.

The title compound was prepared in a manner analogous to 1-(4-amino-2-{1-[6-(3-dimethylamino-propyl)-pyridine-3-sulfonyl]-piperidin-4-ylamino}-thiazol-5-yl)-1-(2,6-difluoro-phenyl)-methanone (Example I10). 1-(4-Amino-2-{1-[6-(3-pyrrolidin-1-yl-prop-2-ynyl)-pyridine-3-sulfonyl]-piperidin-4-ylamino}-thiazol-5-yl)-1-(2,6-difluoro-phenyl)-methanone (200 mg, 0.34 mmol) was hydrogenated and purified via preparative HPLC to provide 114 mg of yellow solid in 57% yield.

¹H NMR (DMSO-d₆): δ 9.58 (br, 1H), 8.85 (d, 2H, J=2.0 Hz), 8.34 (s, 1H), 8.12 (dd, 1H, J=2.0, 8.1 Hz), 8.01 (br, 2H), 7.60 (d, 1H, J=8.1 Hz), 7.50 (m, 1H), 7.16 (dd, 2H, J=7.7, 8.0 Hz), 3.64-3.48 (m, 4H), 3.26-3.16 (m, 2H), 3.10-2.91 (m, 4H), 2.72-2.58 (m, 1H), 2.18-1.82 (m, 8H), 1.64-1.47 (m, 2H). HRFABMS: Calcd. For C₂₇H₃₂F₂N₆O₃S₂ (MH⁺): 591.2018. Found: 590.2041. Anal. Calcd. for C₂₇H₃₂F₂N₆O₃S₂.1.0 H₂O.2.2 CF₃COOH: C, 43.88; H, 4.24; N, 9.78; S, 7.46. Found: C, 43.85; H, 4.21; N, 9.69; S, 7.58.

### Example I14

### 1-(4-Amino-2-{1-[6-(3-piperidin-1-yl-propyl)-pyridine-3-sulfonyl]-piperidin-4-ylamino}-thiazol-5-yl)-1-(2,6-difluoro-phenyl)-methanone Trifluoroacetic Acid Salt.

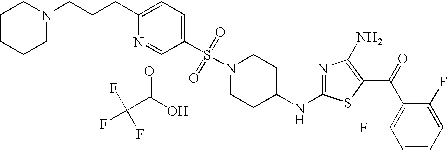 1-(4-Amino-2-{1-[6-(3-piperidin-1-yl-prop-1-ynyl)-pyridine-3-sulfonyl]-piperidin-4-ylamino}-thiazol-5yl)-1-(2,6-difluorophenyl)-methanone

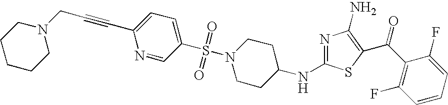

The title intermediate was prepared in a manner analogous to that for 1-(4-amino-2-{1-[4-(3-dimethylamino-prop-1-ynyl)-benzenesulfonyl]-piperidin-4-ylamino}-thiazol-5-yl)-1-(2,6-difluoro-phenyl)-methanone (Example I9). 1-{4-Amino-2-[1-(6-chloro-pyridine-3-sulfonyl)-piperidin-4-ylamino]-thiazol-5-yl}-1-(2,6-difluoro-phenyl)-methanone and 1-prop-2-ynyl-piperidine (Viola, et al., J. Org. Chem., Vol. 58, pp. 5067-75 (1993)) were coupled to provide 445 mg of yellow solid in 74% yield.

¹H NMR (DMSO-d₆): δ 10.10 (br, 1H), 8.92 (s, 1H), 8.23 (dd, 1H, J=2.4, 8.3 Hz), 7.99 (br, 2H), 7.90 (d, 1H, J=8.3 Hz), 7.48 (m, 1H), 7.14 (dd, 2H, J=7.7, 8.1 Hz), 4.46 (s, 2H), 3.62-3.48 (m, 4H), 3.10-2.96 (m, 2H), 2.73-2.61 (m, 1H), 2.00-1.83 (m, 4H), 1.80-1.61 (m, 3H), 1.59-1.42 (m, 3H). LCESIMS (MH⁺): 601.10.

The title compound was prepared in a manner analogous to 1-(4-amino-2-{l-[6-(3-dimethylamino-propyl)-pyridine-3-sulfonyl]-piperidin-4-ylamino}-thiazol-5-yl)-1-(2,6-difluoro-phenyl)-methanone, (Example I10). 1-(4-Amino-2-{1-[6-(3-piperidin-1-yl-prop-1-ynyl)-pyridine-3-sulfonyl]-piperidin-4-ylamino}-thiazol-5-yl)-1-(2,6-difluoro-phenyl)-methanone was hydrogenated and purified via preparative HPLC to provide 200 mg of white solid in 91% yield.

¹H NMR (DMSO-d₆): 9.05 (br, 1H), 8.81 (d, 2H, J=2.1 Hz), 8.10 (dd, 1H, J=2.1, 8.2 Hz), 7.99 (br, 2H), 7.58 (d, 1H, J=8.2 Hz), 7.47 (m, 1H), 7.14 (dd, 2H, J=7.6, 8.1 Hz), 3.55-3.39 (m, 4H), 3.14-3.04 (m, 2H), 2.96-2.89 (m, 4H), 2.17-2.04 (m, 2H), 2.00-1.88 (m, 2H), 1.86-1.75 (m, 2H), 1.75-1.30 (m, 7H). HRMALDIMS. Calcd. for C₂₈H₃₅F₂N₆O₃S₂ (MH⁺): 605.2175. Found: 605.2159. Anal. Calcd. for C₂₈H₃₄F₂N₆O₃S₂.1.0 H₂O2.5 TFA: C, 43.66; H, 4.27; N, 9.26; S, 7.06. Found: C, 43.53; H, 4.32; N, 9.19; S, 7.58.

### Example I15

### {4-Amino-2-[1-(6-vinyl-pyridine-3-sulfonyl)-piperidin-4-ylamino]-thiazol-5-yl}-(2,6-difluoro-phenyl)-methanone

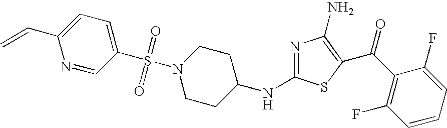

A solution of {4-amino-2-[1-(6-chloro-pyridine-3-sulfonyl)-piperidin-4-ylamino]-thiazol-5-yl}-(2,6-difluoro-phenyl)-methanone (Example F21; 1.00 g, 1.95 mmol) in dioxane (40 ml) was degassed and argon purged, then PdCl₂(PPh₃)₂ (273 mg, 0.40 mmol), tributyl vinyltin (1.7 ml, 5.85 mmol), and 2,6-di-tert-butyl-4-methylphenol (20 mg) were added. The mixture stirred at 100° C. for three and half hours, allowed to cool, solvent was evaporated, and the resultant residue was purified by column chromatography to provide 0.81 g of yellow solid in 82% yield.

¹H NMR (DMSO-d₆): δ 8.84 (s, 1H), 8.12 (d, 1H, J=8.3 Hz), 8.01 (bs, 2H), 7.76(d, 1H, J=8.3 Hz), 7.48 (m, 1H), 7.14 (dd, 2H, J=7.6, 7.9 Hz), 6.94 (dd, 1H, J=11.5, 17.4 Hz), 6.44 (d, 1H, J=17.4 Hz), 5.70 (d, 1H, J=11.5 Hz). ESIMS (M+H⁺): 506.

### Example I16

### {4-Amino-2-[1-(2-vinyl-pyrimidine-5-sulfonyl)-piperidin-4-ylamino]-thiazol-5-yl}-(2,6-difluoro-phenyl)-methanone

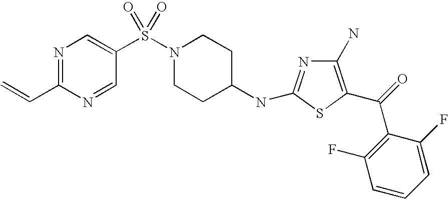

The title compound was prepared in manner similar to that of Example I15 from (4-amino-2-{1-[2-(4-methyl-piperazin-1-yl)-pyrimindin-5-sulfonyl]-piperidin-4-ylamino}-thiazol-5-yl)-(2,6-difluoro-phenyl)-methanone (Example F47).

¹H NMR (DMSO-d₆): δ 9.10 (s, 2H), 8.01 (bs, 2H), 7.52(m, 1H), 7.48 (m, 1H), 7.18 (m, 2H), 6.96 (dd, 1H, J=11.5, 17.4 Hz), 6.72 (d, 1H, J=17.4 Hz), 5.70 (d, 1H, J=11.5 Hz), 3.52 (m, 2H), 2.74 (m, 2H), 1.94 (m, 2H), 1.56 (m, 2H). LC-ESIMS (M+H⁺): 507.
Method J:

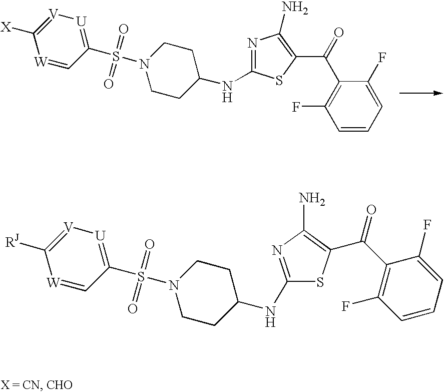

### Example J1

### 1-[4-Amino-2-{1-[4-(1-methyl-4,5-dihydro-1H-imidazol-2-yl)-benzenesulfonyl]-piperidin-4-ylamino}-thiazol-5-yl)-1-(2,6-difluoro-phenyl)-methanone.

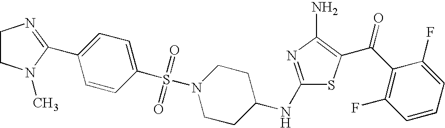

The title compound was prepared as follows. A solution of 4-{4-[4-amino-5-[1-(2,6-difluoro-benzoyl)-thiazol-2-ylamino]-piperidine-1-sulfonyl)benzaldehyde (Example F43; 100 mg, 0.200 mmol), N-methylethylenediamine (176 ul, 2.00 mmol), and sulfur (50 mg) in absolute ethanol (20 ml) refluxed for 12 hours. The solvent was removed and a solution of the resultant residue in ethyl acetate was washed with sat. NaHCO₃ (30 ml×3), dried MgSO₄, filtered, and concentrated. Column chromatography with EtOAc/hexane (2/1) provided 34 mg of a white powder in 31% yield.

¹H NMR (CD₃OD): δ 8.94-8.87 (m, 2H), 8.80-8.72 (m, 2H), 7.50-7.36 (m, 1H), 7.05-6.96 (m, 2H), 3.93-3.84 (m, 2H), 3.72-3.56 (m, 5H), 2.88 (s, 3H), 2.71-2.58 (m, 2H), 2.12-2.00 (m, 2H), 1.73-1.56 (m, 2H). ESIMS (MH⁺): 561. Anal. Calcd for C₂₅H₂₆F₂N₆O₃S₂.0.5 H₂O: C, 52.71; H, 4.78; N, 14.75; S, 11.26. Found: C, 52.39; H, 4.89; N, 14.63; S, 11.01.

### Example J2

### 1-(4-Amino-2-{1-[4-(5,5-dimethyl-4,5-dihydro-1H-imidazol-2-yl)-benzenesulfonyl]-piperidin-4-ylamino}-thiazol-5-yl)-1-(2,6-difluoro-phenyl)-methanone.

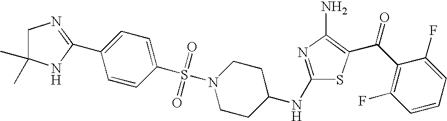

The title compound was prepared as follows. A mixture of 4-{4-[4-amino-5-[1-(2,6-difluoro-benzoyl)-thiazol-2-ylamino]-piperidine-1-sulfonyl)benzaldehyde (Example F43; 200 mg, 0.400 mmol), 2-methyl-propane-1,2-diamine (170 mg, 2.00 mmol), and NaHSO₃ (80 mg, 0.6 mmol) in DMF (5 ml) was heated at 100° C. for one hour. The solvent was removed under reduced pressure. A solution of the resultant residue in ethyl acetate was washed with water, dried over MgSO₄, and concentrated in vacuo. The residue was triturated with ethyl ether and filtered to give 150 mg of a white powder in 65% yield.

¹H NMR (DMSO-d₆): δ 7.88 (d, 2H, J=8.2 Hz), 7.76 (d, 2H, J=8.2 Hz), 7.3 (m, 1H), 6.70 (m, 2H), 3.54 (m, 3H), 3.44 (s, 2H), 2.50 (m, 2H), 2.00 (m, 2H), 1.50 (m, 2H), 1.26 (s, 3H). LCESIMS(MH⁺): 575 Anal. Calcd. For C₂₆H₂₈F₂N₆O₃S₂.0.40 EtOAc: C, 54.35; H, 5.16; N, 13.78; S, 10.51. Found: C, 53.99; H, 5.28; N, 13.66; S, 10.77.

### Example J3

### 4-(4-{4-Amino-5-[1-(2,6-difluoro-phenyl)-methanoyl]-thiazol-2-ylamino}-piperidine-1-sulfonyl)-benzamidine

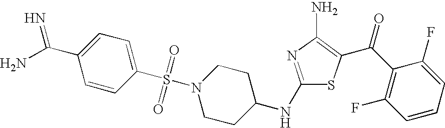

The title compound was prepared as follows. Through a suspension of 4-{4-[4-amino-5-(2,6-difluoro-benzoyl)-thiazol-2-ylamino]-piperidine-1-sulfonyl}-benzonitrile (Example F18; 500 mg, 1.00 mmol) in anhydrous EtOH (30 ml) at 0° C. was passed dry HCl(g) for 15 minutes. The reaction flask was sealed and stirred at ambient temperature for 28 hours. The solvent was removed under reduced pressure and the resultant residue taken up in ethanol (30 ml). Ammonium carbonate (455 mg, 4.95 mmol) was added and the mixture stirred for another 28 hours. The solvent was removed and a solution of the resultant residue in ethyl acetate was washed with sat. NaHCO₃, dried over MgSO₄, filtered, and concentrated. Preparative TLC (2 mm) purification (2% (58% NH₄OH)/15% MeOH/CH₂Cl₂) afforded 120 mg of a yellow solid in 25% yield.

¹H NMR (DMSO-d₆): δ 8.05 (d, 2H, J=8.5 Hz), 7.92 (d, 2H, J=8.6 Hz), 7.52-7.42 (m, 1H, J=8.4 Hz), 7.15 (dd, 2H, J=7.6, 8.2 Hz), 3.58 (d, 2H, J=11.6 Hz), 2.66-2.52 (m, 2H), 1.98-1.88 (m, 2H),1.58-1.44(m,2H). HRMALDIMS. Calcd. for C₂₄H₂₆F₂N₅O₂S (MH⁺): 486.1770. Found: 486.1783. Anal. Calcd. for C₂₄H₂₅F₂N₅O₂S.0.6 H₂O.0.5 NH₄OH.0.8 CH₂Cl₂: C, 44.39; H, 4.46; N, 14.76; S, 10.40. Found: C, 44.09; H, 4.72; N, 14.48; S, 10.50.

### Example J4

### 1-(4-Amino-2-{1-[4-(1H-tetrazol-5-yl)-benzenesulfonyl]-piperidin-4ylamino}-thiazol-5-yl)-1-(2,6-difluoro-phenyl)-methanone Trifluoroacetic Acid Salt.

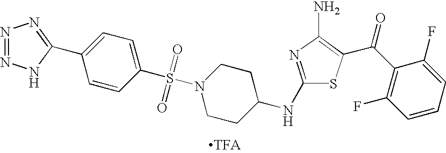

The title compound was prepared as follows. A mixture of 4-{4-[4-amino-5-(2,6-difluoro-benzoyl)-thiazol-2-ylamino]-piperidine-1-sulfonyl}-benzonitrile (Example F18; 250 mg, 0.500 mmol), NaN₃ (0.12 g, 2.0 mmol), and NH₄Cl (0.20 g, 4.0 mmol) in DMF (10 ml) was heated at 70° C. for 60 minutes. The solvent was removed under reduced pressure and a solution of the resultant residue in ethyl acetate was washed with water and concentrated. Purification via preparative HPLC provided 88 mg of solid in 32% yield.

¹H NMR (DMSO-d₆): δ 8.78 (bs, 1H), 8.30 (d, 2H, J=8.3 Hz), 8.11-7.90 (d, 2H, J=8.3 Hz), 7.55-7.40 (m, 1H), 7.13 (t, 2H, J=7.9 Hz), 3.58-3.42 (m, 3H), 2.72-2.58 (m, 2H), 1.98-1.88 (m, 2H), 1.61-1.43 (m, 2H). HRMALDIMS. Calcd. For C₂₂H₂₁F₂N₈O₃S₂ (MH⁺): 547.1141. Found: 547.1157. Anal. Calcd. For C₂₂H₂₀F₂N₈O₃S₂.0.80 TFA: C, 44.44; H, 3.29; N, 17.57; S, 10.05. Found: C, 44.25; H, 3.47; N, 17.50; S, 10.00.

### Example J5

### 1-(4-Amino-2-{1-[4-(4,5-dihydro-oxazol-2-yl)-benzenesulfonyl]-piperidin-4-ylamino}-thiazol-5-yl)-1-(2,6-difluoro-phenyl)-methanone.

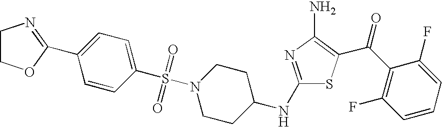

The title compound was prepared as follows. A mixture of 4-{4-[4-amino-5-(2,6-difluoro-benzoyl)-thiazol-2-ylamino]-piperidine-1-sulfonyl)benzonitrile (Example F18; 200 mg, 0.400 mmol), 2-amino-ethanol (488 mg, 8.00 mmol), and ZnCl₂ (100 mg) in chlorobenzene (10 ml) refluxed for 4 hours. The resultant solution was diluted with ethyl acetate, washed with 0.1 N NaOH, dried over MgSO₄, filtered, and concentrated. Column chromatography with CH₂Cl₂/EtOAc/MeOH (5/10/1) afforded 115 mg of a white powder in 51% yield.

¹H NMR (DMSO-d₆): δ 8.04 (d, 2H, J=8.2 Hz), 7.78 (d, 2H, J=8.2 Hz), 7.30 (m, 1H), 6.90 (m, 2H), 4.45 (t, 2H, J=8.5 Hz), 4.00 (t, 2H, J=8.5 Hz), 3.60-3.56 (m, 3H), 2.55-2.51 (m, 2H), 2.06-2.18 (m, 2H), 1.54-1.48 (m, 2H). LC-ESIMS (MH⁺): 548 Anal. Calcd. for C₂₄H₂₃F₂N₅O₄S₂: C, 52.64; H, 4.23; N, 12.79; S, 11.71. Found: C, 52.50; H, 4.38; N, 12.81; S, 11.66.

### Example J6

### 1-{4-Amino-2-[1-(4-pyrrolidin-1-ylmethyl-benzenesulfonyl)-piperidin-4-ylamino]-thiazol-5-yl}-1-(2,6-difluoro-phenyl)-methanone Hydrochloride.

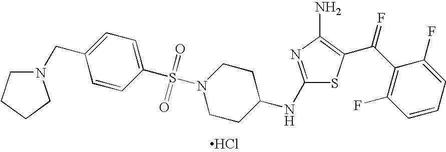

The title compound was prepared as follows. A mixture of pyrrolidine (0.50 ml, 6.0 mmol), 4-{4-[4-amino-5-[1-(2,6-difluoro-benzoyl)-thiazol-2-ylamino]-piperidine-1-sulfonyl}-benzaldehyde (Example F43; 510 mg, 1.00 mmol), sodium cyanoborohydride (NaBH₃CN; 0.04 g, 0.7 mmol), tricaprylylmethylammonium chloride (Aliquat 336, 0.32 ml, 0.70 mmol), 3 Å molecular sieves, 2.5 N HCl in CH₃OH (0.8 ml, 2 mmol), and CH₂Cl₂ (15 ml) stirred at room temperature for 18 hours. The mixture was filtered, and the filtrate concentrated in vacuo. The residue was taken up in H₂O (15 ml) and extracted with ethyl ether. The extracts were dried over MgSO₄ and evaporated to dryness. Purification via preparative HPLC and treatment of the fractions with HCl provided the desired product in 45% yield.

¹H NMR (CD₃OD): δ 7.91 (d, 2H, J=8.4 Hz), 7.82 (d, 2H, J=8.4 Hz), 7.60 (m, 1H), 7.15 (t, 2H, J=8.1 Hz), 4.53 (s, 2H), 3.78-3.68 (m, 2H), 3.61-3.51 (m, 2H), 3.30-3.15 (m, 3H), 2.56 (t, 2H, J=11.1 Hz), 2.28-2.02 (m, 6H), 1.75-1.53 (m, 2H). HRFABMS: Calcd. for C₂₆H₃₀F₂N₅O₃S₂ (MH⁺): 562.1752. Found: 562.1743. Anal. Calcd. For C₂₆H₂₉F₂N₅O₃S₂.1.40 HCl.1.69 H₂O: C, 48.55; H, 5.29, N, 10.89; S, 9.97. Found: C, 48.55; H, 5.42; N, 10.85; S, 9.60.

### Example J7

### 1-(4-Amino-2-{1-[4-methyl-piperazin-1-ylmethyl)-benzenesulfonyl]-piperidin-4-ylamino}-thiazol-5-yl)-1-(2,6-difluoro-phenyl)-methanone Hydrochloride Salt.

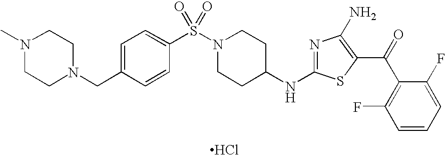

The title compound was prepared in a manner similar to that for Example J6.

¹H NMR (DMSO-d₆): δ 8.78 (bs, 1H), 8.18 (bs, 2H), 7.82 (bs, 4H), 7.60-7.45 (m, 1H), 7.22 (t, 2H, J=15.9 Hz), 4.20-3.98 (m, 3H), 2.68-3.52 (m, 6H), 3.40-3.15 (m, 4H), 2.88 (s, 3H), 2.70-2.60 (m, 2H), 2.08-1.91 (m, 2H), 1.68-1.52 (m, 2H). LC-ESIMS: C₂₇H₃₃F₂N₆O₃S₂ (MH⁺): 591. Anal. Calcd. For C₂₇H₃₂F₂N₆O₃S₂.2.70 HCl.1.40 H₂O: C, 45.39; H, 5.29; N, 11.63; S, 8.98. Found: C, 45.43; H, 5.45; N, 11.63; S, 8.74.

### Example J8

### 1-{4-Amino-2-[1-(4-morpholin4-ylmethyl-benzenzsulfonyl)-piperidin-4-ylamino]-thiazol-5-yl}-1-(2,6-difluoro-phenyl)-methanone Hydrochloride Salt.

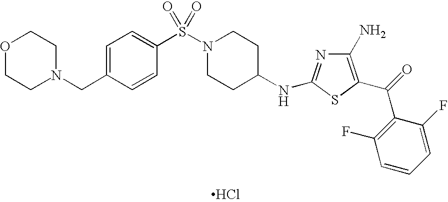

The title compound was prepared in a manner similar to that for Example J6.

¹H NMR (DMSO-d₆): δ 8.88 (bs, 1H), 8.18 (bs, 2H), 8.17-8.02 bs, 2H), 7.95-7.82 (m, 4H), 7.62-7.48 (m, 1H), 7.22 (t, 2H, J=15.9 Hz), 4.52 (s, 2H), 4.08-3.96 (m, 2H), 3.92-3.78 (m, 3H), 3.58-3.50 (m, 2H), 3.38-3.10 (m, 4H), 2.84-2.65 (m, 2H), 2.10-1.90 (m, 2H), 1.68-1.50 (m, 2H). HRMALDIMS: Calcd. for C₂₆H₃₀F₂N₅O₄S₂ (MH⁺): 578.1707. Found: 578.1720. Anal. Calcd. For C₂₆H₂₉F₂N₅O₄S₂.1.60 HCl.0.30 CH₃CN.0.60 H₂O: C, 48.47; H, 5.00; N, 11.26; S, 9.73. Found: C, 48.52; H, 5.26; N,.11.09; S, 9.47.

### Example J9

### 1-{4-Amino-2-[1-(4-{[(2-dimethylamino-ethyl)-methyl-amino]-methyl}-benzenesulfonyl)-piperidin-4-ylamino]-thiazol-5-yl}-1-(2,6-difluoro-phenyl)-methanone Hydrochloride Salt.

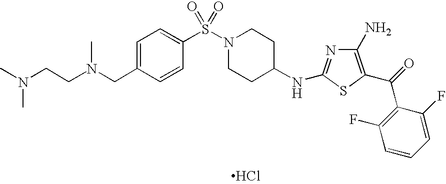

The title compound was prepared in a manner similar to that for Example J6.

¹H NMR (DMSO-d₆): δ 8.88 (bs, 1H), 8.18 (bs, 2H), 8.94-8.82 (m, 4H), 7.68-7.52 (m, 1H), 7.22 (t, 2H, J=15.9 Hz), 4.36 (s, 2H), 3.68-3.35 (m, 7H), 2.93 (s, 6H), 2.68 (s, 3H), 2.08-1.94 (m, 2H), 1.68-1.52 (m, 2H). HRMALDIMS: C₂₇H₃₅F₂N₆O₃S₂ (MH⁺): 593.2180. Found: 593.2189. Anal. Calcd. For C₂₇H₃₄F₂N₆O₃S₂.2HCl.2H₂O: C, 46.21; H, 5.75; N, 11.98; S, 9.14. Found: C, 46.37; H, 5.78; N, 11.98; S 9.05.

### Example J10

### 1-{4-Amino-2-{1-[4-(3,5-dimethyl-piperazin-1-ylmethyl)-benzenesulfonyl]-piperidin-4-ylamino}-thiazol-5-yl)-1-(2,6-difluoro-phenyl)-methanone Hydrochloride Salt.

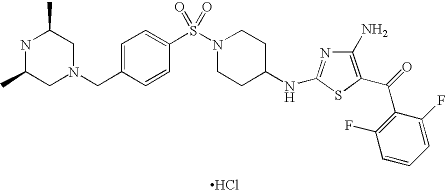

The title compound was prepared in a manner similar to that for Example J6.

¹H NMR (DMSO-d₆): δ 8.82 (bs, 1H), 8.12 (bs, 2H), 8.80-8.61 (m, 4H), 7.58-7.42 (m, 1H), 7.15 (t, 2H, J=15.9 Hz), 3.90-3.81 (m, 3H), 3.58-3.25 (m, 4H), 3.05 (d, 2H, J=11.7 Hz), 2.25 (t, 2H, J=11.9 Hz), 1.98-1.85 (m, 2H), 1.58-1.45 (m, 2H). HRMALDIMS: C₂₈H₃₅F₂N₆O₃S₂ (MH⁺): 605.2180. Found: 605.2157. Anal. Calcd. For C₂₈H₃₄F₂N₆O₃S₂.2.5 HCl.H₂O: C, 47.11; H, 5.44; N, 11.77; S, 8.98. Found: C, 47.11; H, 5.44; N, 11.61; S, 9.03.
Method K:

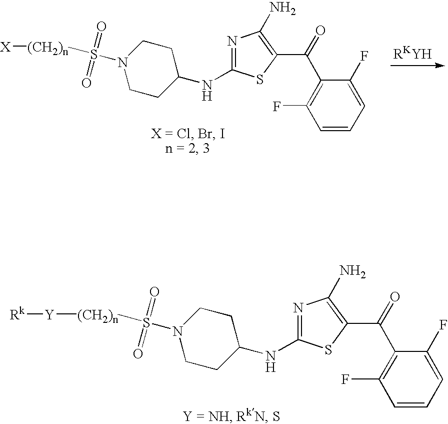

### Example K1

### 1-(4-Amino-2-{1-[3-(3,5-cis-dimethylpiperazin-1-yl)-propane-1-sulfonyl]-piperidin-4-ylamino}-thiazol-5-yl)-1-(2,6-d ifluoro-phenyl)-methanone

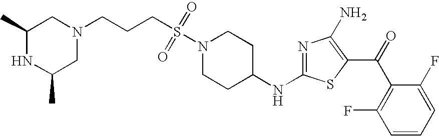

The title compound was prepared as follows. To a solution of 1-{4-amino-2-[1-(3-iodopropane-1-sulfonyl)-piperidin-4-ylamino]-thiazol-5-yl)-1-(2,6-difluoro-phenyl)-methanone (Example F45; 200 mg, 0.350 mmol) in DMF (5ml) were added sequentially diisopropylethylamine (1 ml) and cis-2,6-dimethylpiperazine (200 mg, 1.75 mmol). The mixture stirred at ambient temperature for 4 hours, then was poured into water (500 ml) and extracted with EtOAc. The organic extracts were dried over Na₂SO₄ and concentrated in vacuo to provide 75 mg of product as a pale yellow solid in 38% yield.

¹H NMR (DMSO-d₆): δ 8.78 (bs, 1H), 8.03 (s, 2H), 7.48 (tt, 1H, J=6.8, 8.2 Hz), 7.15 (dd, 2H, J=7.6, 8.2 Hz), 3.59-3.44 (m, 2H), 3.01 (t, 2H, J=7.8 Hz), 2.97-2.84 (m, 3H), 2.79-2.56 (m, 4H), 2.30 (t, 2H, J=6.8 Hz), 2.01-1.84 (m, 2H), 1.77 (tt, 2H, J=6.8, 7.8Hz), 1.58-1.36 (m, 4H), 0.91 (d, 6H, J=6.2 Hz). Anal. Calcd. for C₂₄H₃₄F₂N₆O₃S₂.0.8 H₂O.0.2 EtOAc: C, 50.62; H, 6.39; N, 14.17. Found: C, 50.95; H, 6.31; N, 13.88.

The compounds of the following Examples K2 through K16 were prepared in a manner similar to that for Example K1 from 1-{4-amino-2-[1-(3-iodopropane-1-sulfonyl)-piperidin-4-ylamino]-thiazol-5-yl}-1-(2,6-difluoro-phenyl)-methanone (Example F45) and corresponding amines.

### Example K2

### 1-{4-Amino-2-[1-(3-imidazol-1-yl-propane-1-sulfonyl)-piperidin-4-ylamino}-thiazol-5-yl 1-(2,6-difluoro-phenyl)-methanone.

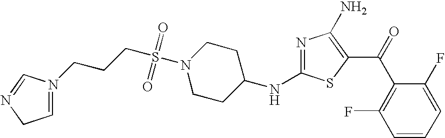

¹H NMR (DMSO-d₆): δ 8.79 (br, 1H), 8.03 (s, 2H), 7.62 (s, 1H), 7.49 (tt, 1H, J=7.0, 8.2 Hz), 7.18 (s, 1H), 7.15 (dd, 2H, d, J=7.8, 8.2 Hz), 6.90 (s, 1H), 4.06 (t, 2H, J=6.8 Hz), 3.50 (m, 2H), 3.0 (m, 5H), 2.08 (tt, 2H, J=6.8, 7.3 Hz), 1.80 (m, 2H), 1.50 (m, 2H) Anal. Calcd. for C₂₁H₂₄F₂N₆O₃S₂.0.5 H₂O.0.25 EtOAc: C, 48.78; H, 5.03; N, 15.52. Found: C, 48.53; H, 4.81; N, 15.64.

### Example K3

### 1-{4-Amino-2-[1-(3-triazol-1-yl-propane-1-sulfonyl)-piperidin-4-ylamino]-thiazol-5-yl}-1-(2,6-difluoro-phenyl)-methanone.

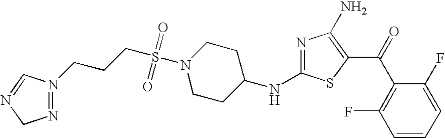

¹H NMR (DMSO-d₆): δ 8.78 (br, 1H), 8.50 (s, 1H), 8.03 (br, 2H), 7.97 (s, 1H), 7.49 (tt, 1H, J=6.5, 8.4 Hz), 7.18 (s, 1H), 7.15 (dd, 2H, J=7.8, 8.2 Hz), 4.29 (t, 2H, J=7.0 Hz), 3.55 (m, 2H), 3.04 (t, 2H, J=7.6 Hz), 2.90 (m, 3H), 2.16 (tt, 2H, J=7.0, 7.6 Hz), 1.95 (m, 2H), 1.50 (m, 2H). Anal. Calcd. for C₂₀H₂₃F₂N₇O₃S₂.0.6 H₂O: C, 45.98; H, 4.67; N, 18.77. Found: C, 45.85; H, 4.69; N, 18.51.

### Example K4

### 1-(4-Amino-2-{1-[3-(dimethylamino)propane-1-sulfonyl]-piperidin-4-ylamino}-thiazol-5-yl)-1-(2,6-difluoro-phenyl)-methanone.

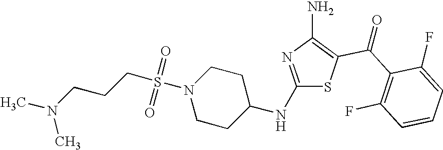

¹H NMR (DMSO-d₆): δ 8.79 (bs, 1H), 8.04 (s, 2H), 7.48 (tt, 1H, J=6.8, 8.2 Hz), 7.15 (dd, 2H, J=7.6, 8.2 Hz), 3.57-3.44 (m, 2H), 3.01 (t, 2H, J=7.7 Hz), 2.96-2.85 (m, 3H), 2.31 (t, 2H, J=6.6 Hz), 2.13 (s, 6H), 2.00-1.86 (m, 2H), 1.76 (t, 2H, J=6.6, 7.7 Hz), 1.56-1.38 (m, 2H). Anal. Calcd. for C₂₀H₂₇F₂N₅O₃S₂.0.5 H₂O.0.25 EtOAc: C, 48.63; H, 5.83; N, 13.50. Found: C, 48.74; H, 5.57; N, 13.64.

### Example K5

### 1-(4-Amino-2-{1-[3-(3,4-dihydro-1H-isoquinolin-2-yl)propane-1-sulfonyl]-piperidin-4-ylamino}-thiazol-5-yl)-1-(2,6-difluoro-phenyl)-methanone.

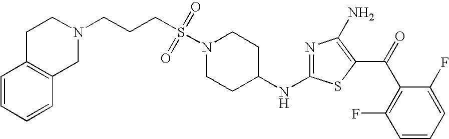

¹H NMR (DMSO-d₆): δ 8.78 (bs, 1H), 8.03 (s, 2H), 7.48 (tt, 1H, J=6.9, 8.2 Hz), 7.15 (dd, 2H, J=7.9, 8.2 Hz), 7.11-7.01 (m, 4H), 3.61-3.46 (m, 4H), 3.07 (t, 2H, J=7.6 Hz), 3.01-2.85 (m, 3H), 2.79 (t, 2H, J=5.8 Hz), 2.64 (t, 2H, J=5.8 Hz), 2.54 (t, 2H, J=6.9 Hz), 2.02-1.81 (m, 4H), 1.56-1.38 (m, 2H). Anal. Calcd. for C₂₇H₃₁F₂N₅O₃S₂: C, 56.33; H, 5.43; N, 12.17. Found: C, 56.10; H, 5.66; N, 11.87.

### Example K6

### 1-(4-Amino-2-{1-[3-(cyclopropylmethyl-propyl-amino)propane-1-sulfonyl]-piperidin-4-ylamino}-thiazol-5-yl)-1-(2,6-difluoro-phenyl)-methanone.

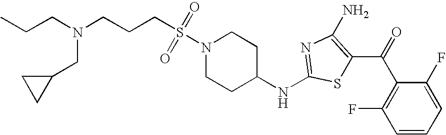

¹H NMR (DMSO-d₆): δ 8.79 (bs, 1H), 8.03 (s, 2H), 7.48 (tt,1H, J=6.9, 8.2Hz), 7.15 (dd, 2H, J=7.8, 8.2Hz), 3.59-3.45 (m, 2H), 3.11-2.84 (m, 6H), 2.43-2.17 (m, 3H), 2.02-1.65 (m, 5H), 1.57-1.29 (m, 5H), 0.92-0.75 (m, 4H), 0.52-0.34 (m, 2H), 0.14-0.00 (m, 2H). Anal. Calcd. for C₂₅H₃₅F₂N₅O₃S₂.0.5 H₂O: C, 53.17; H, 6.43; N, 12.40. Found: C, 53.19; H, 6.35; N, 12.05.

### Example K7

### 1-(4-Amino-2-{1-[3-(piperidin-1-yl)propane-1-sulfonyl]-piperidin-4-ylamino}-thiazol-5-yl)-1-(2,6-difluoro-phenyl)-methanone.

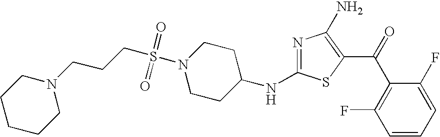

¹H NMR (DMSO-d₆): δ 8.77 (bs, 1H), 8.03 (s, 2H), 7.48 (tt, 1H, J=6.9, 8.2 Hz), 7.15 (dd, 2H, J=7.9, 8.2 Hz), 3.58-3.44 (m, 2H), 3.01 (t, 2H, J=7.4 Hz), 2.97-2.84 (m, 3H), 2.39-2.19 (m, 5H), 2.01-1.85 (m, 2H), 1.77 (tt, 2H, J=6.7, 7.4 Hz), 1.57-1.27 (m, 9H). Anal. Calcd. for C₂₃H₃₁F₂N₅O₃S₂: C, 52.35; H, 5:92; N, 13.27. Found: C, 52.12; H, 6.17; N, 12.92.

### Example K8

### 1-(4-Amino-2-{1-[3-(pyrrolidin-1-yl)propane-1 sulfonyl]-piperidin-4-ylamino}-thiazol-5-yl)-1-(2,6-difluoro-phenyl)-methanone.

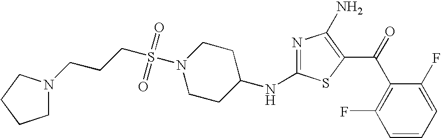

¹H NMR (DMSO-d₆): δ 8.79 (bs, 1H), 8.03 (s, 2H), 7.48 (tt, 1H, J=6.9, 8.2 Hz), 7.15 (dd, 2H, J=7.9, 8.2 Hz), 3.58-3.44 (m, 2H), 3.04 (t, 2H, J=7.7 Hz), 2.98-2.85 (m, 4H), 2.46-2.33 (m, 5H), 2.02-1.87 (m, 2H), 1.80 (tt, 2H, J=6.7, 7.7 Hz), 1.73-1.61 (m, 4H), 1.56-1.38 (m, 2H). Anal. Calcd. for C₂₂H₂₉F₂N₅O₃S₂.0.5 H₂O: C, 50.56; H, 5.79; N, 13.40. Found: C, 50.77; H, 5.85; N, 13.01.

### Example K9

### 1-(4-Amino-2-{1-[3-(2,5-dihydropyrrol-1-yl)propane-1-sulfonyl]-piperidin-4-ylamino}-thiazol-5-yl)-1-(2,6-difluoro-phenyl)-methanone.

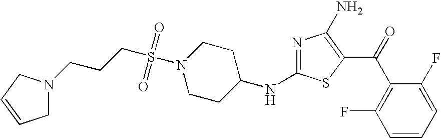

¹H NMR (DMSO-d₆): δ 8.79 (bs, 1H), 8.03 (s, 2H), 7.48 (tt, 1H, J=6.9, 8.2 Hz), 7.15 (dd, 2H, J=7.9, 8.2 Hz), 5.78 (s, 2H), 3.57-3.44 (m, 2H), 3.38 (s, 4H), 3.05 (t, 2H, J=7.7 Hz), 2.99-2.85 (m, 3H), 2.64 (t, 2H, J=6.8 Hz), 2.01-1.86 (m, 2H), 1.78 (tt, 2H, J=6.8, 7.7 Hz), 1.56-1.38 (m, 2H). Anal. Calcd. for C₂₂H₂₇F₂N₅O₃S₂: C, 51,65; H, 5.32; N, 13.69. Found: C, 51.95; H, 5.43; N, 13.50.

### Example K10

### 1-(4-Amino-2-{1-[3-([cis/trans]-octahydro-1H-isoquinolin-2-yl)propane-1-sulfonyl]-piperidin-4-ylamino}-thiazol-5-yl)-1-(2,6-difluoro-phenyl)-methanone.

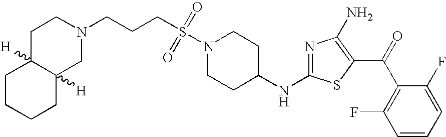

¹H NMR (DMSO-d₆): δ 8.78 bs, 1H), 8.03 (s, 2H), 7.48 (tt, 1H, J=6.9, 8.2 Hz), 7.15 (dd, 2H, J=7.9, 8.2 Hz), 3.58-3.44 (m, 2H), 3.09-2.86 (m, 5H), 2.83-2.61 (m, 2H), 2.37-2.21 (m, 2H), 2.03-0.76 (m, 20H). Anal. Calcd. for C₂₇H₃₇F₂N₅O₃S₂.0.25 EtOAc: C, 55.70; H, 6.51; N, 11.60. Found: C, 55.82; H, 6.62; N, 11.69.

### Example K11

### 1-(4-Amino-2-{1-[3-(3,6-dihydro-2H-pyridin-1-yl)propane-1-sulfonyl]-piperidin-4-ylamino}-thiazol-5-yl)-1-(2,6-difluoro-phenyl)-methanone.

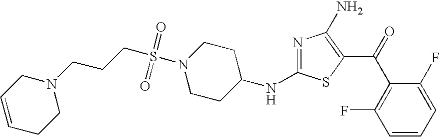

¹H NMR (DMSO-d₆): δ 8.78 bs, 1H), 8.03 (s, 2H), 7.48 (tt, 1H, J=6.9, 8.2 Hz), 7.15 (dd, 2H, J=7.9, 8.2 Hz), 5.72-5.58 (m, 2H), 3.58-3.44 (m, 2H), 3.03 (t, 2H, J=7.7 Hz), 2.98-2.80 (m, 5H), 2.47-2.36 (m, 4H), 2.11-1.87 (m, 4H), 1.81 (tt, 2H, J=7.4, 7.7 Hz), 1.56-1.38 (m, 2H). Anal. Calcd. for C₂₃H₂₉F₂N₅O₃S₂.0.25 EtOAc: C, 52.63; H, 5.71; N, 12.79. Found: C, 52.37; H, 5.75; N, 13.09.

### Example K12

### 1-(4-Amino-2-{1-[3-(morpholin-4-yl)propane-1-sulfonyl]-piperidin-4-ylamino}-thiazol-5-yl)-1-(2,6-difluoro-phenyl)-methanone.

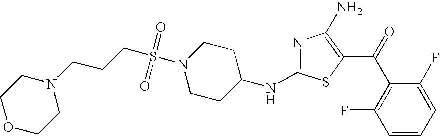

¹H NMR (DMSO-d₆): δ 8.81 bs, 1H), 8.03 (s, 2H), 7.49 (tt, 1H, J=6.9, 8.2 Hz), 7.15 (dd, 2H, J=7.9, 8.2 Hz), 3.63-3.44 (m, 6H), 3.03 (t, 2H, J=7.6 Hz), 2.99-2.85 (m, 3H), 2.41-2.24 (m, 6H), 2.01-1.86 (m, 2H), 1.79 (tt, 2H, J=6.6, 7.6 Hz), 1.56-1.38 (m, 2H). Anal. Calcd. for C₂₂H₂₉F₂N₅O₄S₂.0.25 H₂O: C, 49.47; H, 5.57; N, 13.11. Found: C, 49.55; H, 5.71; N, 12.82.

### Example K13

### 1-(4-Amino-2-{1-[3-(thiomorpholin-4-yl)propane-1-sulfonyl]-piperidin-4-ylamino}-thiazol-5-yl)-1-(2,6-difluoro-phenyl)-methanone.

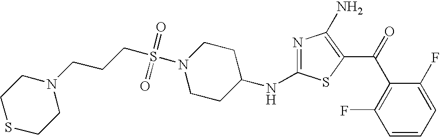

¹H NMR (DMSO-d₆): δ 8.80 bs, 1H), 8.03 (s, 2H), 7.49 (tt, 1H, J=6.9, 8.2 Hz), 7.16 (dd, 2H, J=7.9, 8.2 Hz), 3.60-3.45 (m, 2H), 3.01 (t, 2H, J=7.7 Hz), 2.97-2.86 (m, 3H), 2.72-2.54 (m, 6H), 2.39 (t, 2H, J=7.0 Hz), 2.03-1.86 (m, 2H), 1.77 (tt, 2H, J=7.0, 7.7 Hz), 1.56-1.38 (m, 2H), 1.05-0.89 (m, 2H). Anal. Calcd. for C₂₂H₂₉F₂N₅O₃S₃: C, 48.42; H, 5.36; N, 12.83. Found: C, 48.15; H, 5.48; N, 12.45.

### Example K14

### 1-(4-Amino-2-{1-[3-(3,3-dimethylpiperazin-1-yl)propane-1-sulfonyl]-piperidin-4-ylamino}-thiazol-5-yl)-1-(2,6-difluoro-phenyl)-methanone.

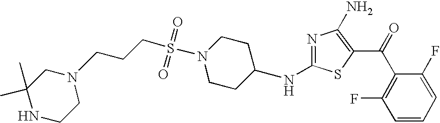

Prepared in a manner similar to that for Example K1 from 1-{4-amino-2-[1-(3-iodopropane-1-sulfonyl)-piperidin-4-ylamino]-thiazol-5-yl}-1-(2,6-difluoro-phenyl)-methanone (Example F45) and 2,2-dimethylpiperazine (Bφgesφ, et al., J. Med. Chem., Vol. 38, pp. 4380-4392 (1995)).

¹H NMR (DMSO-d₆): δ 8.75 bs, 1H), 8.03 (s, 2H), 7.48 (tt,1H, J=6.8, 8.2 Hz), 7.15 (dd, 2H, J=7.9, 8.2 Hz), 3.57-3.44 (m, 2H), 3.03 (t, 2H, J=7.6 Hz), 2.98-2.83 (m, 3H), 2.72 (t, 2H, J=4.8 Hz), 2.27 (t, 2H, J=6.7 Hz), 2.23-2.13 (m, 2H), 2.06-1.86 (m, 4H), 1.77 (tt, 2H, J=6.7, 7.6 Hz), 1.56-1.38 (m, 2H), 1.03 (s, 6H). Anal. Calcd. for C₂₄H₃₄F₂N₆O₃S₂.0.5 H₂O.0.15 Et₂O: C, 51.22; H, 6.38; N, 14.57. Found: C, 51.05; H, 6.12; N, 14.27.

### Example K15

### 1-(4-Amino-2-{1-[3-(4-ethylpiperazin-1-yl)propane-1-sulfonyl]-piperidin-4-ylamino}-thiazol-5-yl)-1-(2,6-difluoro-phenyl)-methanone.

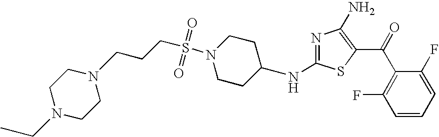

¹H NMR (DMSO-d₆): δ 8.80 bs, 1H), 8.03 (s, 2H), 7.48 (tt, 1H, J=6.8, 8.2 Hz), 7.15 (dd, 2H, J=7.9, 8.2 Hz), 3.57-3.44 (m, 2H), 3.02 (t, 2H, J=7.6 Hz), 2.98-2.85 (m, 3H), 2.44-2.18 (m, 12H), 2.00-1.86 (m, 2H), 1.77 (tt, 2H, J=6.7, 7.6 Hz), 1.56-1.38 (m, 2H), 0.97 (t, 3H, J=7.0 Hz). Anal. Calcd. for C₂₄H₃₄F₂N₆O₃S₂.1.0 H₂O: C, 50.16; H, 6.31; N, 14.62. Found: C, 50.17; H, 6.16; N, 14.34.

### Example K16

### 1-(4-Amino-2-{1-[3-(4-methylpiperazin-1-yl)propane-1-sulfonyl]-piperidin-4-ylamino}-thiazol-5-yl)-1-(2,6-difluoro-phenyl)-methanone.

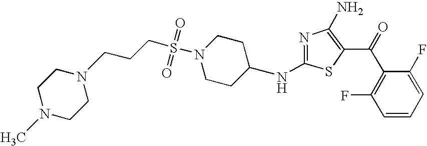

¹H NMR (DMSO-d₆): δ 8.78 (bs, 1H), 8.03 (s, 2H), 7.48 (tt, 1H, J=6.8, 8.2n Hz), 7.15 (dd, 2H, J=7.9, 8.2 Hz), 3.58-3.44 (m, 2H), 3.01 (t, 2H, J=7.7 Hz), 2.97-2.85 (m, 3H), 2.42-2.22 (m, 10H), 2.14 (s, 3H), 2.01-1.86 (m, 2H), 1.77 (tt, 2H, J=6.7, 7.7 Hz), 1.56-1.38 (m, 2H). Anal. Calcd. for C₂₃H₃₂F₂N₆O₃S₂.0.4 H₂O.0.2 Et₂O: C, 50.62; H, 6.21; N, 14.88. Found: C, 50.61; H, 6.26; N, 14.49.

### Example K17

### 1-(4-{4-Amino-5-[1-(2,6-difluorophenyl)methanoyl]-thiazol-2-ylamino}-piperidine-1-sulfonyl)butyronitrile.

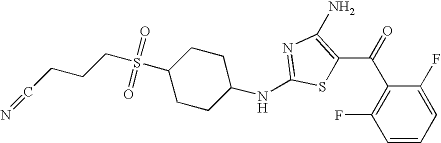

The title compound was prepared in a manner analogous to that for Example K1 from 1-{4-amino-2-[1-(3-iodopropane-1-sulfonyl)-piperidin-4-ylamino]-thiazol-5-yl}-1-(2,6-difluoro-phenyl)-methanone and potassium cyanide.

¹H NMR (DMSO-d₆): δ 8.78 (bs, 1H), 8.03 (s, 2H), 7.48 (tt, 1H, J=6.9, 8.2 Hz), 7.15 (dd, 2H, J=7.9, 8.2 Hz), 3.60-3.46 (m, 2H), 3.11 (t, 2H, J=7.5 Hz), 3.02-2.85 (m, 3H), 2.63 (t, 2H, J=7.2 Hz), 2.03-1.86 (m, 4H), 1.56-1.38 (m, 2H). Anal. Calcd. for C₁₉H₂₁F₂N₅O₃S₂.0.5 H₂O: C, 47.69; H, 4.63; N, 14.64. Found: C, 47.65; H, 4.71; N, 14.64.

### Example K18

### 1-(4-Amino-2-{1-[3-(1H-tetrazol-5-yl)-propane-1-sulfonyl]-piperidin-4-ylamino}-thiazol-5-yl)-1-(2,6-difluoro-phenyl)-methanone.

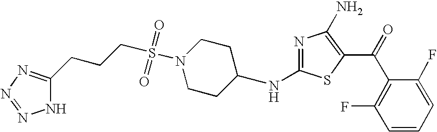

The title compound was prepared as follows. To a solution of 4-(4-{4-amino-5-[1-(2,6-difluorophenyl)methanoyl]-thiazol-2-ylamino}-piperidine-1-sulfonyl)butyronitrile (Example K17; 200 mg, 4.30 mmol) in DMF (5 ml) were added sodium azide (760 mg, 11.7 mmol) and ammonium chloride (760 mg, 14.2 mmol). The resultant mixture was heated at 65° C. for 4 days. This mixture was supplemented with additional sodium azide (500 mg, 7.7 mmol) and ammonium chloride (500 mg, 9.3 mmol). After 7 days at 65° C., the mixture was poured into water and extracted with ethyl acetate. The organic layer was separated, dried over Na₂SO₄, and concentrated in vacuo to provide 80 mg of a yellow solid in 37% yield.

¹H NMR (DMSO-d₆): δ 8.78 (bs, 1H), 8.76 (bs 1H), 8.03 (s, 2H), 7.48 (tt, 1H, J=6.8, 8.2 Hz), 7.15 (dd, 2H, J=7.9, 8.2 Hz), 3.60-3.46 (m, 2H), 3.16 (t, 2H, J=7.5 Hz), 3.02 (t, 2H, J=7.6 Hz), 2.97-2.85 (m, 3H), 2.10 (tt, 2H, J=7.5, 7.6 Hz), 2.01-1.86 (m, 2H), 1.56-1.38 (m, 2H). Anal. Calcd. for C₁₉H₂₂F₂N₈O₃S₂.1.0 H₂O.0.3 Et₂O: C, 43.89; H, 4.92; N, 20.27. Found: C, 44.05; H, 4.49; N, 19.93.

### Example K19

### 1-{4-Amino-2-[1-(3-azetidin-1-yl-propane-1-sulfonyl)-piperidin-4-ylamino]-thiazol-5-yl}-1-(2,6-difluoro-phenyl)-methanone.

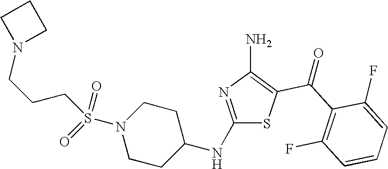

The title compound was prepared in a manner similar to that of Example K1 from 1-{4-amino-2-[1-(3-iodopropane-1-sulfonyl)-piperidin-4-ylamino]-thiazol5-yl}-1-(2,6-difluoro-phenyl)-methanone (Example F45) and azetidine

¹HNMR (DMSO d₆): δ 8.79 (s, 1H), 8.03 (s, 2H), 7.53-7.43 (m, 1H), 7.17-7.11 (m, 2H) 3.52-3.41 (m, 2H), 3.08-2.72 (m, 4H), 2.40-2.36 (m, 2H), 1.97-1.88 (m, 4H), 1.64-1.40 (m, 4H). Anal. Calcd for C₂₁H₂₉F₂N₅O₃S₂.0.1H₂O: C, 50.28; H, 5.42; N, 13.96. Found: C, 50.10; H, 5.57; N, 13.60.

### Example K20

### N-{1-[3-(4-{4-Amino-5-[1-(2,6-difluoro-phenyl)-methanoyl]-thiazol-2-ylamino}-piperidine-1-sulfonyl)-propyl]-pyrrolidin-3-yl}-N-methyl-acetamide

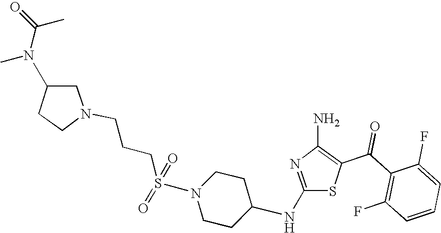

The title compound was prepared in a manner similar to that of Example K1 from 1-{4-amino-2-[1-(3-iodopropane-1-sulfonyl)-piperidin-4-ylamino]-thiazol-5-yl}-1-(2,6-difluoro-phenyl)-methanone (Example F45) and N-methyl-N-pyrrolidin-3-yl-acetamide

¹HNMR (DMSO d₆): δ 8.79 (s, 1H), 8.02 (s, 2H), 7.51-7.45 (m, 1H), 7.17-7.12 (m, 2H) 3.53-3.49 (m, 2H), 3.28 (s, 3H), 3.07-2.93 (m, 4H), 2.10 (s, 3H), 2.07-1.82 (m, 4H), 1.97-1.88 (m, 4H), 1.64-1.40 (m, 4H). Anal. Calcd for C₂₅H₃₄F₂N₆O₄S₂.1 H₂O: C, 50.28; H, 5.98; N, 13.93. Found: C, 50.60; H, 5.77; N, 13.63.

### Example K21

### 1-(4-Amino-2-{1-[3-(pyridin-2-ylsulfanyl)-propane-1-sulfonyl]-piperidin-4-ylamino}-thiazol-5-yl)-1-(2,6-difluoro-phenyl)-methanone

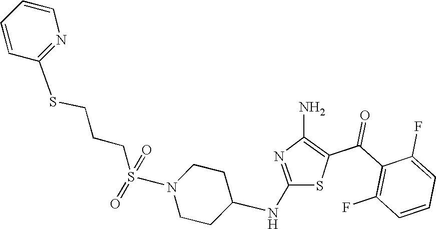

The title compound was prepared in a manner similar to that of Example K1 from 1-{4-amino-2-[1-(3-iodopropane-1-sulfonyl)-piperidin-4-ylamino]-thiazol-5-yl}-1-(2,6-difluoro-phenyl)-methanone (Example F45) and pyridine-2-thiol.

¹H NMR (DMSO-d₆): δ 8.43 (d, J=4.2Hz, 1H), 8.03 (s, 2H), 7.65-7.60 (m, 1H), 7.48-7.43 (m, 1H), 7.30 (d, J=8.1 Hz, 1H ),7.17-7.08 (m, 1H) 3.54-3.49 (m, 2H), 3.41-3.20 (m, 4H), 3.18-2.72 (m, 2H), 2.07-1.91 (m, 4H), 1.51-1.41 (m, 2H). Anal. Calcd for C₂₃H₂₅F₂N₅O₃S₃.0.1H₂O: C, 49.70; H, 4.51; N, 12.59. Found: C, 50.04; H, 4.80; N, 12.19.

### Example K22

### 1-(4-Amino-2-{1-[3-(1-methyl-1H-imidazol-2-ylsulfanyl)-propane-1-sulfonyl]-piperidin-4-ylamino}-thiazol-5-yl)-1-(2,6-difluoro-phenyl)-methanone.

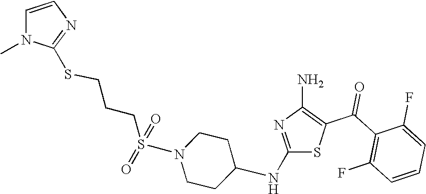

The title compound was prepared in a manner similar to that of Example K1 from 1-{4-amino-2-[1-(3-iodopropane-1-sulfonyl)-piperidin-4-ylamino]-thiazol-5-yl}-1-(2,6-difluoro-phenyl)-methanone (Example F45) and 1-methyl-1-H-imidazole-2-thiol.

¹H NMR (DMSO-d₆): δ 8.78 (bs, 1H), 8.03 (s, 2H), 7.50-7.43 (m, 1H), 7.23 (s, 1H), 7.17-7.12(m, 2H), 6.92 (s, 1H), 3.98 (s, 3H), 3.57-3.52 (m, 2H), 3.27-3.25 (m, 2H), 3.18-2301 (m, 4H), 2.07-1.91 (m, 4H), 1.51-1.41 (m, 2H). Anal. Calcd for C₂₂H₂₆F₂N₆O₃S₃.0.1 Et₂O: C, 47.65; H, 4.73; N, 14.89. Found: C, 47.89; H, 5.13; N, 14.60.

### Example K23

### 1-(4-Amino-2-{1-[3-(pyridin-4-ylsulfanyl)-propane-1-sulfonyl]-piperidin-4-ylamino}-thiazol-5-yl)-1-(2,6-difluoro-phenyl)-methanone.

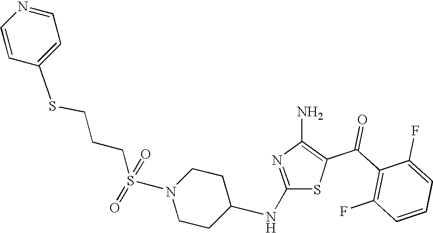

The title compound was prepared in a manner similar to that of Example K1 from 1-{4-amino-2-[1-(3-iodopropane-1-sulfonyl)-piperidin-4-ylamino]-thiazol-5-yl}-1-(2,6-difluoro-phenyl)-methanone (Example F45) and pyridine-4-thiol.

¹H NMR (DMSO d-6): δ 8.77 (bs, 1H), 8.38 (d, J=6.0Hz, 2H), 7.53-7.43 (m, 1H), 7.23 (s, 1H), 7.29(d,J=6.0 Hz, 2H), 7.18-7.13 (m, 2H), 3.53-3.49 (m, 2H), 3.21-3.15 (m, 4H), 2.95-2.88 (m, 2H), 2.07-1.93 (m, 4H), 1.51-1.41 (m, 2H). Anal. Calcd for C₂₃H₂₅F₂N₅O₃S₃: C, 49.89; H, 4.73; N, 12.57. Found: C, 50.32; H, 4.73; N, 12.57.

### Example K24

### 1-(4-Amino-2-{1-[3-(2-dimethylamino-ethylsulfanyl)-propane-1-sulfonyl]-piperidin-4-ylamino}-thiazol-5-yl)-1-(2,6-difluoro-phenyl)-methanone.

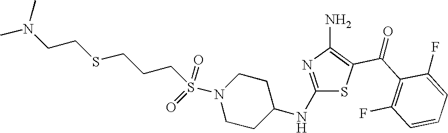

The title compound was prepared in a manner similar to that of Example K1 from 1-{4-amino-2-[1-(3-iodopropane-1-sulfonyl)-piperidin-4-ylamino]-thiazol-5-yl}-1-(2,6-difluoro-phenyl)-methanone (Example F45) and 2-dimethylamino-ethanethiol.

¹H NMR (DMSO-d₆): δ 8.79 (bs, 1H), 8.03 (s, 2H), 7.53-7.43 (m, 1H), 7.17-7.12 (m, 2H), 3.54-3.40 (m, 2H), 3.13-2.97 (m, 2H), 2.93-2.88 (m, 2H), 2.71-2.63 (m, 2H), 2.63-2.56 (m,4H), 2.18 (s, 6H),1.95-1.83 (m, 4H), 1.51-1.41 (m, 2H). Anal. Calcd for C₂₂H₃₁F₂N₅O₃S₃.0.5H₂O: C, 47.46; H, 5.79; N, 12.58. Found: C, 47.60; H, 5.75; N, 12.38.

### Example K25

### (4-Amino-2-{1-[2-(2-methoxy-ethylamino}-ethanesulfonyl]-piperidin-4-ylamino}-thiazol-5-yl)-(2,6-difluoro-phenyl)-methanone Trifluoroacetic Acid Salt.

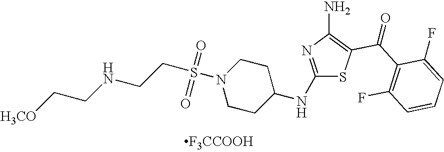

The title compound was prepared as follows. A solution of [4-amino-2-(1-ethenesulfonyl-piperidin-4-ylamino}-thiazol-5-yl]-(2,6-difluoro-phenyl)-methanone (70mg, 0.16 mmol; Example F55) and 2-methoxyethylamine (37 mg, 0.49 mmol) in THF (0.5 ml) stirred at 60° C. for 3 hours, solvent was removed in vacuo, and resultant residue purified via preparative HPLC to give 36 mg of white powder in 45% yield.

¹H NMR (DMSO-d₆): δ 8.82 (bs, 1H), 8.70 (bs, 1H), 8.06 (bs, 2H), 7.50 (m, 1H), 7.18 (dd, 2H, J=7.6, 8.1 Hz), 3.32 (s, 3H), 2.99 (dd, 2H, J=10.6, 12.2 Hz). HRESIMS. Calcd for C₂₀H₂₈F₂N₅O₄S₂ (M+H⁺): 504.1551. Found: 504.1567. Anal. Calcd. for C₂₀H₂₇F₂N₅O₄S₂.0.8 H₂O.2.0 TFA: C, 38.64; H, 4.13; N, 9.39; S, 8.60. Found: C, 38.87; H, 4.28; N, 9.43; S, 8.52.

### Example K26

### (4-Amino-2-{1-[2-(cis/trans-2,5-dimethyl-pyrrolidin-1-yl)-ethanesulfonyl]-piperidin-4-ylamino}-thiazol-5-yl)-(2,6-difluoro-phenyl)-methanone Trifluoroacetic Acid Salt.

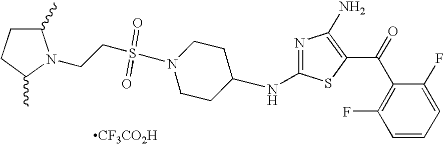

The title compound was prepared in a manner analogous to Example K25. [4-Amino-2-(1-ethenesulfonyl-piperidin-4-ylamino}-thiazol-5-yl]-(2,6-difluoro-phenyl)-methanone (Example F55; 100 mg, 0.16 mmol) and cis/trans-2,5-dimethylpyrrolidine (68 mg, 0.69 mmol) gave 85 mg (yield 70%) of white powder in 70% yield.

¹H NMR (DMSO-d₆): δ 9.11 (bs, 1H), 8.03 (bs, 2H), 7.48 (m, 1H), 7.15 (dd, 2H, J=7.7, 8.0 Hz), 3.00 (dd, 2H, J=10.2, 11.5 Hz), 1.32 (d, 6H, J=6.5 Hz). HRESIMS. Calcd for C₂₃H₃₂F₂N₅O₃S₂ (M+H⁺): 528.1915. Found: 528.1918. Anal. Calcd. for C₂₃H₃₁F₂N₅O₃S₂.2.0 TFA: C, 42.91; H, 4.40; N, 9.27; S, 8.49. Found: C, 42.68; H, 4.58; N, 9.14; S, 8.56.

### Example K27

### (4-Amino-2-{1-[2-(cis/trans-2,5-dimethyl-2,5-dihydro-pyrrol-1-yl)-ethanesulfonyl]-piperidin-4-ylamino}-thiazol-5-yl)-(2,6-difluoro-phenyl)-methanone Trifluoroacetic Acid Salt.

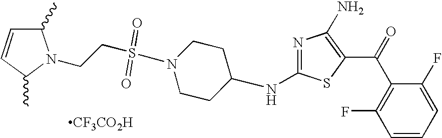

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 100 mg, 0.16 mmol) and 2,5-dimethylpyrroline (68 mg, 0.70 mmol) gave 81 mg of white powder in 67% yield, which displayed a mixture of cis/trans isomers by ¹H NMR.

¹H NMR (DMSO-d₆): δ 9.50 (bs, 1H), 8.80 (bs, 1H), 7.99 (bs, 2H), 7.45 (m, 1H), 7.12 (dd, 2H, J=7.7, 7.9 Hz), 6.01 (s, 0.4H), 5.81 (s, 1.6H), 2.98 (dd, 2H, J=10.2, 12.1 Hz). ESMS (M+H⁺): 526. Anal. Calcd. for C₂₃H₂₉F₂N₅O₃S₂.2.0 TFA: C, 43.03; H, 4.15; N, 9.29; S, 8.51. Found: C, 42.90; H, 4.36; N, 9.19; S, 8.47.

### Example K28

### (4-Amino-2-{1-[2-(2-pyrrolidin-1-yl-ethylamino)-ethanesulfonyl]-piperidin-4-ylamino}-thiazol-5-yl)-(2,6-difluoro-phenyl)-methanone Trifluoroacetic Acid Salt.

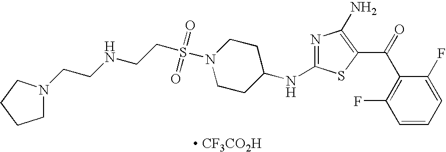

The title compound was prepared in a manner analogous to Example K25. [4-Amino-2-(1-ethenesulfonyl-piperidin-4-ylamino)-thiazol-5-yl]-(2,6-difluoro-phenyl)-methanone (80 mg, 0.19 mmol; Example F55) and 1-(2-aminoethyl)-pyrrolidine (64 mg, 0.56 mmol) gave 51 mg of white powder in 49% yield.

¹H NMR (DMSO-d₆): δ 9.40 (bs, 1H), 8.97 (bs, 1H), 8.16 (bs, 2H), 7.60 (m, 1H), 7.26 (dd, 2H, J=7.8, 7.9 Hz), 3.11 (dd, 4H, J=10.3, 11.6 Hz). HRESIMS. Calcd for C₂₃H₃₃F₂N₆O₃S₂ (M+H⁺): 543.2024. Found: 543.2018. Anal. Calcd. for C₂₃H₃₂F₂N₆O₃S₂.1.0 H₂O.2.5 TFA: C, 39.76; H, 4.35; N, 9.94; S, 7.58. Found: C, 39.53; H, 4.58; N, 10.13; S, 7.88.

### Example K29

### (4-Amino-2-{1-[2-(2-pyrrolidin-1-yl-ethylamino)-ethanesulfonyl]-piperidin-4-ylamino}-thiazol-5-yl)-(2,6-difluoro-phenyl)-methanone Trifluoroacetic Acid Salt.

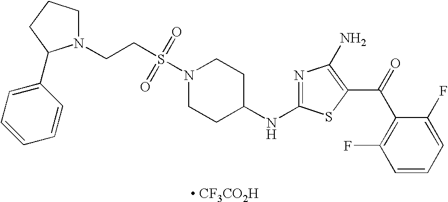

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 80 mg, 0.19 mmol) and 2-phenylpyrrolidine (82 mg, 0.56 mmol) gave 60 mg of white powder in 55% yield.

¹H NMR (DMSO-d₆): δ 10.00 (bs, 1H), 8.81 (bs, 1H), 8.06 (bs, 2H), 7.17 (dd, 2H, J=7.8, 7.9 Hz). HRESIMS. Calcd for C₂₇H₃₂F₂N₅O₃S₂ (M+H³⁰ ): 576.1915. Found: 576.1928. Anal. Calcd. for C₂₇H₃₁F₂N₅O₃S₂.1.9 TFA: C, 46.69; H, 4.19; N, 8.84; S, 8.09. Found: C, 46.33; H, 4.30; N, 8.99; S, 8.32.

### Example K30

### (4-Amino-2-{1-[2-(cyclopentyl-methyl-amino)-ethanesulfonyl]-piperidin-4-ylamino}-thiazol-5-yl)-(2,6-difluoro-phenyl)-methanone Trifluoroacetic Acid Salt.

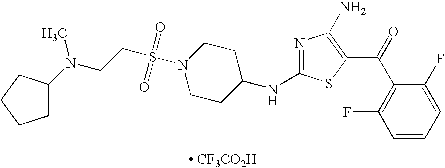

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 80 mg, 0.19 mmol) and N-methylcyclopentylamine (56 mg, 0.56 mmol) gave 72 mg (yield 72%) of white powder in 72% yield.

¹H NMR (DMSO-d₆): δ 9.94 (bs, 1H), 8.90 (bs, 1H), 8.11 (bs, 2H), 7.56 (m, 1H), 7.23 (dd, 2H, J=7.7, 8.0 Hz), 3.06 (dd, 2H, J=10.1, 11.0 Hz), 2.85 (s, 3H). HRESIMS. Calcd for C₂₃H₃₂F₂N₅O₃S₂ (M+H³⁰ ): 528.1915. Found: 528.1919. Anal. Calcd. for C₂₃H₃₁F₂N₅O₃S₂.1.9 TFA: C, 43.25; H, 4.46; N, 9.41; S, 8.62. Found: C, 43.25; H, 4.74; N, 9.43; S, 8.85.

### Example K31

### (4-Amino-2-{1-[2-(1,1-dioxo-tetrahydro-1-lamda-6-thiophen-3-ylamino)-ethanesulfonyl]-piperidin-4-ylamino}-thiazol-5-yl)-(2,6-difluoro-phenyl)-methanone Trifluoroacetic Acid Salt.

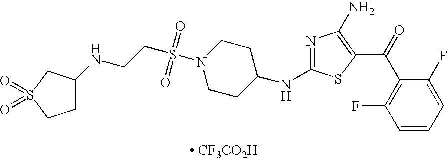

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55;80 mg, 0.19 mmol) and tetrahydro-3-thiophenamine 1,1-dioxide (76 mg, 0.56 mmol) gave 88 mg of white powder in 82% yield.

¹H NMR (DMSO-d₆): δ 9.15 (bs, 1H), 8.81 (bs, 1H), 8.06 (bs, 2H), 7.51 (m, 1H), 7.17 (dd, 2H, J=7.8, 7.9 Hz), 3.00 (dd, 2H, J=10.4, 12.2 Hz). HRESIMS. Calcd for C₂₁H₂₈F₂N₅O₅S₃(M+H³⁰ ): 564.1221. Found: 564.1235. Anal. Calcd. for C₂₁H₂₇F₂N₅O₅S₃.1.0 H₂O.2.0 TFA: C, 37.08; H, 3.86; N, 8.65; S, 11.88. Found: C, 36.92; H, 4.08; N, 8.47; S 11.81.

### Example K32

### (4-Amino-2-{1-[2-(3,6-dihydro-2H-pyridin-1-yl)-ethanesulfonyl]-piperidin-4-ylamino}-thiazol-5-yl)-(2,6-difluoro-phenyl)-methanone Trifluoroacetic Acid Salt.

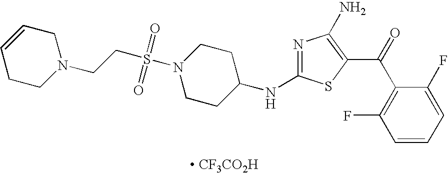

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 100 mg, 0.23 mmol) and 1,2,3,6-tetrahydropyridine (39 mg, 0.47 mmol) gave 61 mg of white powder in 52% yield.

¹H NMR (DMSO-d₆): δ 9.85 (bs, 1H), 8.06 (bs, 2H), 7.51 (m, 1H), 7.18 (dd, 2H, J=7.7, 8.0 Hz), 5.98 (d, 1H, J=10.6 Hz), 5.73 (d, 1H, J=10.6 Hz), 3.15 (m, 1H), 3.01 (dd, 2H, J=11.2, 11.4 Hz). HRESIMS. Calcd for C₂₂H₂₈F₂N₅O₃S₂ (M+H³⁰ ): 512.1602. Found: 512.1594. Anal. Calcd. for C₂₂H₂₇F₂N₅O₃S₂.2.0 TFA: C, 42.22; H, 3.95; N, 9.47; S, 8.67. Found: C, 42.43; H, 4.13; N, 9.58; S, 8.91.

### Example K33

### {4-Amino-2-[1-(2-methylamino-ethanesulfonyl)-piperidin-4-ylamino]-thiazol-5-yl}-(2,6-difluoro-phenyl)-methanone Trifluoroacetic Acid Salt.

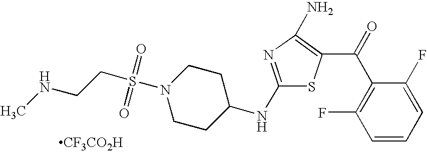

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 100 mg, 0.23 mmol) and methylamine (2 ml of 1.0 M in THF) gave 59 mg of white powder in 56% yield.

¹H NMR (DMSO-d₆): δ 8.82 (bs, 1H), 8.52 (bs, 1H), 8.06 (bs, 2H), 7.51 (m, 1H), 7.17 (dd, 2H, J=7.7, 8.0 Hz), 3.55 (d, 2H, J=12.4 Hz), 3.00 (dd, 2H, J=11.0, 11.1 Hz), 2.62 (t, 3H, J=5.0 Hz). HRESIMS. Calcd for C₁₈H₂₄F₂N₅O₃S₂ (M+H³⁰ ): 460.1289. Found: 460.1281. Anal. Calcd. for C₁₈H₂₃F₂N₅O₃S₂.1.8 TFA: C, 39.03; H, 3.76; N, 10.53; S, 9.65. Found: C, 38.68; H, 3.95; N, 10.40; S, 9.67.

### Example K34

### {4-Amino-2-[1-(2-pyrrol-1-yl-ethanesulfonyl)-piperidin-4-ylamino]-thiazol-5-yl}-(2,6-difluoro-phenyl)-methanone Trifluoroacetic Acid Salt.

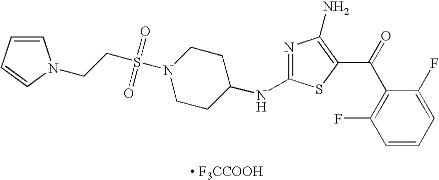

4-Amino-2-(1-ethenesulfonyl-piperidin-4-ylamino)-thiazol-5-yl]-(2,6-difluoro-phenyl)-methanone (Example F55; 50 mg, 0.12 mmol) and KOH (30 mg) stirred in pyrrole (0.1 ml) and CH₃CN (0.5 ml) at 80° C. overnight. The mixture was concentrated in vacuo and purified via preparative HPLC to give 49 mg of white powder in 82% yield.

¹H NMR (DMSO-d₆): δ 8.78 (bs, 1H), 8.07 (bs, 2H), 7.49 (m, 1H), 6.83 (bs, 2H), 5.99 (bs, 2H). HRESIMS. Calcd for C₂₁H₂₄F₂N₅O₃S₂ (M+H³⁰ ): 496.1289. Found: 496.1298. Anal. Calcd. for C₂₁H₂₃F₂N₅O₃S₂.0.4 TFA: C, 48.38; H, 4.36; N, 12.94; S, 11.85. Found: C, 48.15; H, 4.51; N, 12.93; S, 11.72.

### Example K35

### 1-{4-Amino-2-[1-(2-pyrrolidin-1-yl-ethanesulfonyl)-piperidin-4-ylamino]-thiazol-5-yl}-1-(2,6-difluoro-phenyl)-methanone.

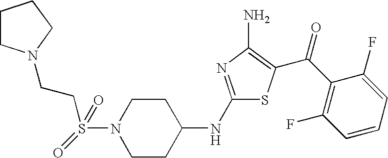

The title compound was prepared in a manner similar to that used to prepare Example K25 from [4-Amino-2-(1-ethenesulfonyl-piperidin-4-ylamino}-thiazol-5-yl]-(2,6-difluoro-phenyl)-methanone (Example F55) and pyrrolidine.

1H NMR (DMSO-d₆): δ 8.79 (bs, 1H), 8.01 (s, 2H), 7.53-7.43 (m, 1H), 7.17-7.14 (m, 2H), 3.55-3.51 (m, 2H), 3.34-3.21 (m, 2H), 2.96-2.89 (m, 2H), 2.75-2.69 (m, 2H), 2.07-1.92 (m,2H), 1.67 (m, 4H), 1.52-1.41 (m, 2H). Anal. Calcd for C₂₂H₃₁F₂N₅O₃S₃.0.1 Et₂O.0.2 H₂O: C, 50.34; H, 5.61; N, 13.72. Found: C, 50.66; H, 5.61; N, 13.33.

### Example K36

### (4-Amino-2-{1-[2-(2,5-dihydro-pyrrol-1-yl)-ethanesulfonyl]-piperidin-4-ylamino}-thiazol-5-yl)-(2,6-difluoro-phenyl)-methanone.

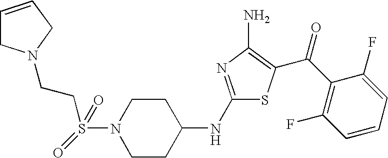

The title compound was prepared in a manner similar to that used to prepare Example X1 from [4-Amino-2-(1-ethenesulfonyl-piperidin-4-ylamino)-thiazol-5-yl]-(2,6-difluoro-phenyl)-methanone (Example F55) and 2,5-dihydro-pyrrole.

¹H NMR (DMSO-d₆): δ 8.75(bs, 1H), 8.05(s, 2H), 7.53-7.43 (m, 1H), 7.18-7.12 (m, 2H), 5.8(s, 2H), 4.10-2.70 (m, 13H), 2.07-1.92 (m,2H), 1.67 (m, 4H), 1.50-1.44 (m, 2H). Anal. Calcd for C₂₁H₂₅F₂N₅O₃S₂: C, 50.69; H, 5.03; N, 14.07. Found: C, 50.96; H, 5.03; N, 13.88.

### Example K37

### (4-Amino-2-{1-[2-(methyl-phenyl-amino)-ethanesulfonyl]-piperidin-4-ylamino}-thiazol-5-yl)-(2,6-difluoro-phenyl)-methanone Trifluoroacetic Acid Salt.

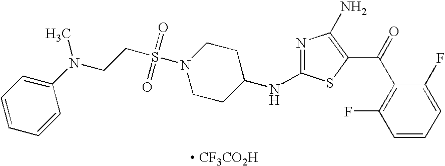

[4-Amino-2-(1-ethenesulfonyl-piperidin-4-ylamino)-thiazol-5-yl]-(2,6-difluoro-phenyl)-methanone (Example F55; 150 mg, 0.44 mmol) and N-methylaniline (238 mg, 2.22 mmol) in CH₃CN (1.0 ml) at 80° C. stirred for 3 days. The mixture was concentrated and purified via preparative HPLC to give 58 mg of white powder in 25% yield.

¹H NMR (DMSO-d₆): δ 8.83 (bs, 1H), 8.11 (bs, 2H), 7.53 (m, 1H), 6.76 (d, 2H, J=8.3 Hz), 6.71 (dd, 2H, J=7.3, 9.5 Hz), 3.76 (dd, 2H, J=7.0, 7.5 Hz), 3.58 (d, 2H, J=12.4 Hz), 3.25 (dd, 2H, J=7.0, 7.5 Hz), 2.99 (dd, 2H, J=11.2, 12.4 Hz), 2.94 (s, 3H). HRESIMS. Calcd for C₂₄H₂₈F₂N₅O₃S₂ (M+H³⁰ ): 536.1602. Found: 526.1597. Anal. Calcd. for C₂₄H₂₇F₂N₅O₃S₂.1.6 TFA: C, 45.50; H, 4.01; N, 9.75; S, 8.93. Found: C, 45.65; H, 4.28; N, 9.55; S, 9.20.

### Example K38

### {4-Amino-2-[1-(2-cyclopentylsulfanyl-ethanesulfonyl)-piperidin-4-ylamino]-thiazol-5-yl}-(2,6-difluoro-phenyl)-methanone Trifluoroacetic Acid Salt.

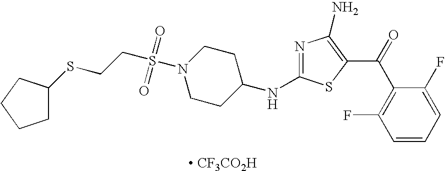

[4-Amino-2-(1-ethenesulfonyl-piperidin-4-ylamino)-thiazol-5-yl]-(2,6-difluoro-phenyl)-methanone (Example F55; 80 mg, 0.19 mmol) and cyclopentyl mercaptan (57 mg, 0.56 mmol) stirred in CH₃CN (0.5 ml) and triethylamine (0.1 ml) at 80° C. for 5 hours. The mixture was concentrated in vacuo and purified by preparative HPLC to give 87 mg of a white powder in 86% yield.

¹H NMR (DMSO-d₆): δ 8.80 (bs, 1H),8.07 (bs, 2H), 7.49 (m, 1H). HRESIMS. Calcd for C₂₂H₂₉F₂N₄O₃S₃ (M+H³⁰ ): 531.1370. Found: 531.1388. Anal. Calcd. for C₂₂H₂₈F₂N₄O₃S₃.0.4 TFA: C, 47.52; H, 4.97; N, 9.72; S, 16.69. Found: C, 47.63; H, 5.11; N, 9.59; S, 16.44.

### Example K39

### (4-Amino-2-{1-[2-(benzyl-methyl-amino)-ethanesulfonyl]-piperidin-4-ylamino}-thiazol-5-yl)-(2,6-difluoro-phenyl)-methanone

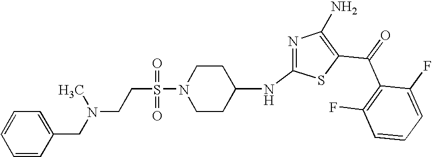

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.58 mmol) and N-benzylmethylamine (212 mg, 1.75 mmol) gave 288 mg of white powder in 90% yield.

¹H NMR (DMSO-d₆): δ 8.98 (bs, 1H), 8.27 (bs, 2H), 7.69 (m, 1H), 7.37 (dd, 2H, J=7.8, 7.8 Hz), 3.52 (s, 2H), 3.43 (dd, 2H, J=6.9, 7.5 Hz), 3.08 (dd, 2H, J=10.4, 10.9 Hz), 2.91 (dd, 2H, J=6.9, 7.5 Hz), 2.33 (s, 3H), 1.69 (d, 1H, J=11.1 Hz), 1.60 (d, 1H, J=9.8 Hz). Anal. Calcd. for C₂₅H₂₉F₂N₅O₃S₂: C, 54.63; H, 5.32; N, 12.74; S, 11.67. Found: C, 54.35; H, 5.30; N, 12.74; S, 11.77.

### Example K40

### (4-Amino-2-{1-[2-(4(cis/trans)-methyl-cyclohexylamino)-ethanesulfonyl]-piperidin-4-ylamino}-thiazol-5-yl)-(2,6-difluoro-phenyl)-methanone

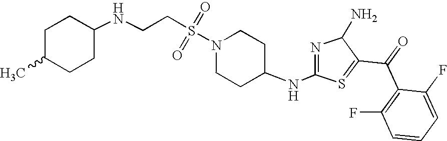

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 220 mg, 0.51 mmol) and 4-methylcyclohexylamine (174 mg, 1.54 mmol) gave 210 mg of white powder in 76% yield.

¹H NMR (DMSO-d₆): δ 8.71 (bs, 1H), 8.08 (bs, 2H), 7.50 (m, 1H), 7.15 (dd, 2H, J=7.5, 7.7 Hz), 3.52 (d, 1H, J=10.6 Hz), 0.84 (d, 3H, J=6.3 Hz). HRESIMS. Calcd for C₂₄H₃₄F₂N₅O₃S₂: 542.2071; Found: 540.2070. Anal. Calcd. for C₂₄H₃₃F₂N₅O₃S₂.0.2 H₂O.0.3 hexane: C, 54.26; H, 6.64; N, 12.26; S, 11.23. Found: C, 53.91; H, 6.59; N, 12.50; S, 11.03.

### Example K41

### (4-Amino-2-{1-[2-(3-methyl-benzylamino)-ethanesulfonyl]-piperidin-4-ylamino}-thiazol-5-yl)-(2,6-difluoro-phenyl)-methanone

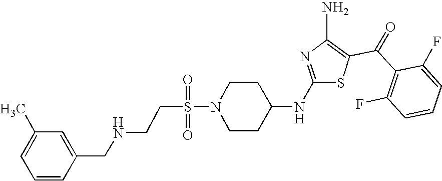

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 220 mg, 0.51 mmol) and 3-methylbenzylamine (187 mg, 1.54 mmol) gave 180 mg of white powder in 64% yield.

¹H NMR (DMSO-d₆): δ 8.73 (bs, 1H), 8.08 (bs, 2H), 7.50 (m, 1H), 3.65 (s, 2H), 3.52 (d, 2H, J=12.1 Hz), 3.18 (dd, 2H, J=6.7, 6.8 Hz), 2.92 (dd, 2H, J=10.1, 11.0 Hz), 2.83 (dd, 2H, J=6.8, 7.0 Hz), 2.28 (s, 3H). HRESIMS. Calcd for C₂₅H₃₀F₂N₅O₃S₂: 550.1758; Found: 550.1764. Anal. Calcd. for C₂₅H₂₉F₂N₅O₃S₂.0.2 Hexane: C, 55.51; H, 5.65; N, 12.35; S, 11.31. Found: C, 55.52; H, 5.73; N, 12.31; S, 11.55.

### Example K42

### (4-Amino-2-{1-[2-(1S-phenyl-propylamino)-ethanesulfonyl]-piperidin-4-ylamino}-thiazol-5-yl)-(2,6-difluoro-phenyl)-methanone

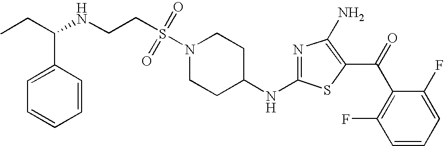

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 220 mg, 0.51 mmol) and (S)(−)-1-phenylpropylamine (208 mg, 1.54 mmol) gave 202 mg of white powder in 70% yield.

¹H NMR (DMSO-d₆): δ 8.82 (bs, 1H), 8.24 (bs, 2H), 7.56 (m, 1H), 2.92 (dd, 2H, J=11.1, 11.5 Hz), 2.69 (dd, 2H, J=7.0, 7.1 Hz), 1.69 (m, 1H), 0.79 (t, 3H, J=7.4 Hz). HRESIMS. Calcd for C₂₆H₃₂F₂N₅O₃S₂: 564.1915; Found: 564.1941. Anal. Calcd. for C₂₆H₃₁F₂N₅O₃S₂.0.4 H₂O: C, 54.70; H, 5.61; N, 12.27; S, 11.23. Found: C, 54.89; H, 5.59; N, 12.27; S, 11.25.

### Example K43

### (4-Amino-2-{1-[2-(3(cis/trans)-methyl-cyclohexylamino)-ethanesulfonyl]-piperidin-4-ylamino}-thiazol-5-yl)-(2,6-difluoro-phenyl)-methanone

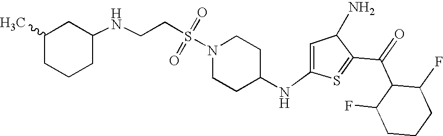

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.58 mmol) and 3-methylcyclohexylamine (198 mg, 1.75 mmol) gave 227 mg of white powder in 72% yield.

¹H NMR (DMSO-d₆): δ 8.83 (bs, 1H), 8.12 (bs, 2H), 7.55 (m, 1H), 7.22 (dd, 2H, J=7.6, 7.8 Hz), 3.57 (d, 2H, J=11.9 Hz), 1.85 (d, 2H, J=11.0 Hz), 0.91 (d, 3H, J=6.5 Hz). HRESIMS. Calcd for C₂₄H₃₃F₂N₅O₃S₂: 542.2071; Found: 542.2075. Anal. Calcd. for C₂₄H₃₃F₂N₅O₃S₂.0.8 H₂O: C, 51.84; H, 6.27; N, 12.59; S, 11.53. Found: C, 51.97; H, 6.22; N, 12.63; S, 11.47.

### Example K44

### 4-Amino-2-{1-[2-(3,3,5-trimethyl-cyclohexylamino)-ethanesulfonyl]-piperidin-4-ylamino}-thiazol-5-yl)-(2,6-difluoro-phenyl)-methanone

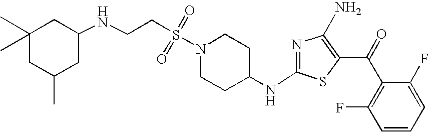

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 220 mg, 0.51 mmol) and 4-methylcyclohexylamine (218 mg, 1.54 mmol) gave 236 mg of white powder in 81% yield.

¹H NMR (DMSO-d₆): δ 8.89 (bs, 1H), 8.19 (bs, 2H), 7.62 (m, 1H), 7.28 (dd, 2H, J=7.7, 7.9 Hz), 3.64 (d, 2H, J=12.1 Hz), 3.23 (dd, 2H, J=6.6, 6.7 Hz), 1.94 (d, 1H, J=12.5 Hz), 0.99 (d, 6H, J=2.7 Hz), 0.96 (d, 3H, J=6.5 Hz), 0.88 (d, 1H, J=11.7 Hz), 0.79(d, 1H, J=12.5 Hz), 0.57 (m, 1H). HRESIMS. Calcd for C₂₆H₃₈F₂N₅O₃S₂: 570.2384; Found: 570.2376. Anal. Calcd. for C₂₆H₃₇F₂N₅O₃S₂.0.5 H₂O.0.5 Hexane: C, 54.82; H, 6.90; N, 11.75; S, 10.76. Found: C, 54.50; H, 6.85; N, 11.66; S, 10.61.

### Example K45

### (4-Amino-2-{1-[2-(2,3-difluoro-benzylamino)-ethanesulfonyl]-piperidin-4-ylamino}-thiazol-5-yl)-(2,6-difluoro-phenyl)-methanone

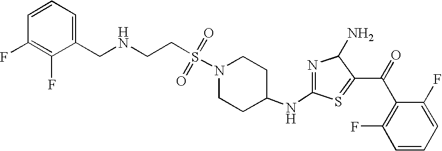

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 220 mg, 0.51 mmol) and 2,3-difluorobenzylamine (220 mg, 1.54 mmol) gave 198 mg of white powder in 68% yield.

¹H NMR (DMSO-d₆): δ 8.78 (bs, 1H), 8.08 (bs, 2H), 7.50 (m, 1H), 3.79 (s, 2H), 3.52 (d, 2H, J=12.7 Hz), 3.33 (s, 2H), 3.20 (dd, 2H, J=6.7, 6.7 Hz). Anal. Calcd. for C₂₄H₂₅F₄N₅O₃S₂: C, 50.43; H, 4.41; N, 12.25; S, 11.22. Found: C, 50.39; H, 4.42; N, 12.37; S, 11.28.

### Example K46

### AG-024360: (4-Amino-2-{1-[2-(1 R-phenyl-propylamino)-ethanesulfonyl]-piperidin-4-ylamino}-thiazol-5-yl)-(2,6-difluoro-phenyl)-methanone

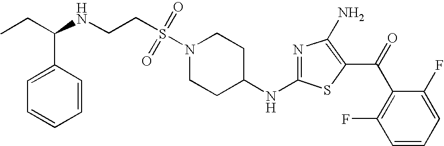

The title compound was prepared in a manner analogous to Example K25. [4-Amino-2-(1-ethenesulfonyl-piperidin-4-ylamino}-thiazol-5-yl]-(2,6-difluoro-phenyl)-methanone (Example F55; 220 mg, 0.51 mmol) and (R)(+)-1-phenylpropylamine (208 mg, 1.54 mmol) gave 213 mg of white powder in 74% yield.

¹H NMR identical to that for Example K42. HRESIMS. Calcd for C₂₆H₃₁F₂N₅O₃S₂: 564.1915; Found: 594.1924. Anal. Calcd. for C₂₆H₃₁F₂N₅O₃S₂.0.5 H₂O: C, 54.53; H, 5.63; N, 12.23; S, 11.20. Found: C, 54.58; H, 5.60; N, 12.14; S, 11.04.

### Example K47

### 4-Amino-2-{1-[2-((S)-1-phenyl-ethylamino)-ethanesulfonyl]-piperidin-4-ylamino}-thiazol-5-yl)-(2,6-difluoro-phenyl)-methanone

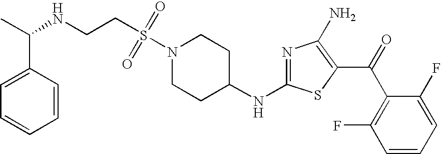

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.74 mmol) and (S)-(−)-1-methylbenzylamine (268 mg, 2.2 mmol) gave 213 mg of yellow powder in 61% yield.

¹H NMR: (DMSO-d₆): 8.79 (bs, 1H), 8.10 (bs, 2H), 7.52 (m, 1H), 7.33 (d, 4H, J=3.8 Hz), 3.54 (q, 1H, 6.6 Hz), 3.50 (d, 2H, J=14.2 Hz), 2.96 (dd, 2H, J=10.4, 10.6 Hz), 1.45 (d, 3H, J=6.6 Hz). Anal. Calcd. for C₂₅H₂₉F₂N₅O₃S₂.0.4 H₂O: C, 53.92; H, 5.39; N, 12.58; S, 11.52. Found: C, 53.97; H, 5.33; N, 12.35; S, 11.40

### Example K48

### {4-Amino-2-[1-(2-benzylamino-ethanesulfonyl)-piperidin-4-ylamino]-thiazol-5-yl}-2,6-difluoro-phenyl)-methanone

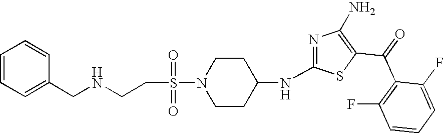

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.74 mmol) and benzylamine (236 mg, 2.2 mmol) gave 280 mg of white powder in 71% yield.

¹H NMR: (DMSO-d₆): 8.79 (bs, 1H), 8.09 (bs, 2H), 7.50 (m, 1H), 7.32 (d, 4H, J=3.6 Hz), 3.54 (s, 2H), 3.50 (d, 2H, J=12.1 Hz), 3.04 (dd, 2H, J=6.4, 6.8 Hz). Anal. Calcd. for C₂₄H₂₇F₂N₅O₃S₂.0.3 H₂O.0.1 heptane: C, 53.84; H, 5.34; N, 12.71; S, 11.64. Found: C, 53.79; H, 5.29; N, 12.65; S, 11.56.

### Example K49

### (4-Amino-2-{1-[2-(1-methyl-1-phenyl-ethylamino)-ethanesulfonyl]-piperidin-4-ylamino}-thiazol-5-yl)-(2,6-difluoro-phenyl)-methanone

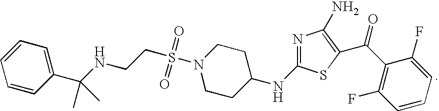

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.74 mmol) and cumylamine (300 mg, 2.22 mmol) gave 256 mg of white powder in 61% yield.

¹H NMR: (DMSO-d₆): 8.74 (bs, 1H), 8.09 (bs, 2H), 7.55 (m, 1H), 7.46 (d, 2H, J=7.3 Hz), 7.33 (dd, 2H, J=7.3, 7.9 Hz), 7.18 (dd, 2H, J=7.3, 8.3 Hz), 3.49 (d, 2H, J=12.2 Hz), 3.02 (dd, 2H, J=6.4, 6.7 Hz), 2.97 (dd, 2H, J=9.0, 9.7 Hz), 1.37 (s, 6H). HRESIMS. Calcd for C₂₆H₃₁F₂N₅O₃S₂: 564.1915; Found: 594.1924. Anal. Calcd. for C₂₆H₃₁F₂N₅O₃S₂.0.2 H₂O.0.2 heptane: C, 56.03; H, 5.94; N, 11.92; S, 10.92. Found: C, 55.96; H, 5.95; N, 11.81; S, 10.82.

### Example K50

### (4-Amino-2-{1-[2-(2,6-difluoro-benzylamino)-ethanesulfonyl]-piperidin-4-ylamino}-thiazol-5-yl)-(2,6-difluoro-phenyl)-methanone

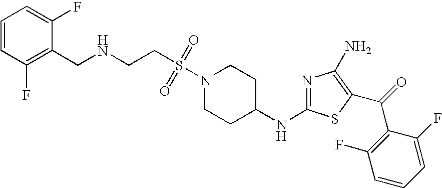

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.740 mmol) and 2,6-difluorobenzylamine (315 mg, 2.22 mmol) gave 278 mg of white powder in 66% yield.

¹H NMR: (DMSO-d₆): 8.78 (bs, 1H), 8.10 (bs, 2H), 7.52 (m, 1H), 7.39 (m, 1H), 7.18 (dd, 2H, J=7.7, 7.9 Hz), 7.09 (dd, 2H, J=7.9, 8.2 Hz), 3.77 (s, 2H), 3.50 (d, 2H, J=12.4 Hz), 3.04 (dd, 2H, J=6.2, 6.9 Hz). HRESIMS. Calcd for C₂₆H₃₁F₂N₅O₃S₂: 564.1915; Found: 594.1924. Anal. Calcd. for C₂₄H₂₅F₄N₅O₃S₂.0.5 H₂O.0.1 heptane: C, 50.23; H, 4.71; N, 11.86; S, 10.86. Found: C, 50.42; H, 4.60; N, 11.76; S, 10.84.

### Example K51

### (4-Amino-2-{1-[2-(2,2-dimethyl-propylamino)-ethanesulfonyl]-piperidin-4-ylamino}-thiazol-5-yl)-(2,6-difluoro-phenyl)-methanone

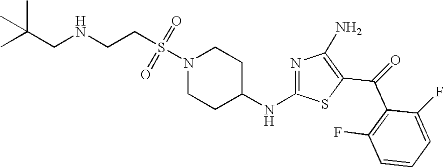

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.74 mmol) and neopentylamine (193 mg, 2.22 mmol) gave 260 mg of white powder in 68% yield.

¹H NMR: (DMSO-d₆): 8.70 (bs, 1H), 8.02 (bs, 2H), 7.40 (m, 1H), 7.12 (dd, 2H, J=7.5, 8.1 Hz), 3.50 (d, 2H, J=12.7 Hz), 3.12 (dd, 2H, J=6.1, 6.6 Hz), 2.21 (s, 2H), 0.80 (s, 9H). Anal. Calcd. for C₂₂H₃₁F₂N₅O₃S₂: C, 51.24; H, 6.06; N, 13.58; S, 12.44. Found: C, 50.97; H, 6.15; N, 13.48; S, 12.26.

### Example K52

### (4-Amino-2-{1-[2-(3-chloro-benzylamino)-ethanesulfonyl]-piperidin-4-ylamino}-thiazol-5-yl)-(2,6-difluoro-phenyl)-methanone

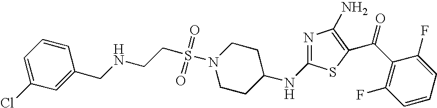

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.74 mmol) and 3-chlorobenzylamine (314 mg, 2.22 mmol) gave 290 mg of white powder in 69% yield.

¹H NMR: (DMSO-d₆): 8.95 (bs, 1H), 8.25 (bs, 2H), 7.68 (m, 1H), 7.58 (s, 1H), 7.35 (dd, 2H, J=7.7, 7.9 Hz), 3.90 (s, 2H), 3.68 (d, 2H, J=12.4 Hz), 3.36 (dd, 2H, J=6.1, 6.4 Hz). Anal. Calcd. for C₂₄H₂₆F₂N₅O₃S₂Cl: C, 50.56; H, 4.60; N, 12.28; S, 11.25. Found: C, 50.48; H, 4.67; N, 12.19; S, 11.17.

### Example K53

### (4-Amino-2-{1-[2-(benzyl-cyclopropylmethyl-amino)-ethanesulfonyl]-piperidin-4-ylamino}-thiazol-5-yl)-(2,6-difluoro-phenyl)-methanone

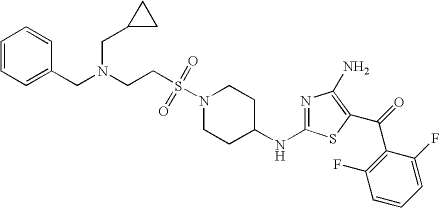

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.74 mmol) and benzyl-cyclopropylmethylamine (358 mg, 2.22 mmol) gave 320 mg of white powder in 73% yield.

¹H NMR: (DMSO-d₆): 8.68 (bs, 1H), 8.01 (bs, 2H), 7.44 (m, 1H), 7.25 (d, 4H, J=3.2 Hz), 7.11(dd, 2H, J=7.7, 8.1 Hz), 3.57 (s, 2H), 3.48 (d, 2H, J=12.1 Hz), 2.00 (d, 2H, J=6.4 Hz), 0.78 (m, 1H), 0.38 (dd, 2H, J=4.4, 9.8 Hz), 0.00 (dd, 2H, J=4.4, 9.6 Hz). HRESIMS. Calcd for C₂₆H₃₁F₂N₅O₃S₂: 564.1915; Found: 594.1924. Anal. Calcd. for C₂₈H₃₃F₂N₅O₃S₂.0.3 H₂O: C, 56.51; H, 5.69; N, 11.77; S, 10.78. Found: C, 56.57; H, 5.66; N, 11.82; S, 10.93.

The starting material for the above was prepared as follows:

### Benzyl-cyclopropylmethylamine

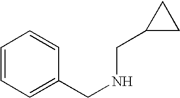

According to a procedure from Tverezovsky, et al, Tetrahedron, Vol. 53, pp. 14773-14792 (1997); (bromomethyl)cyclopropane and benzylamine gave a yellow oil, which was purified via column chromatography with 0.5% (58% NH₄OH)/5% MeOH/CH₂Cl₂ as eluant. The colorless oil displayed an ¹H NMR that matched literature (Harada, et al, Tetrahedron, Vol. 54, pp. 753-766 (1998)) and was used without any further purification.

### Example K54

### (4-Amino-2-{1-[2-(bis-cyclopropylmethyl-amino)-ethanesulfonyl]-piperidin-4-ylamino}-thiazol-5-yl)-(2,6-difluoro-phenyl)-methanone

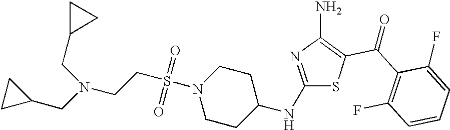

The title compound was prepared in a manner analogous to Example K25. [4-Amino-2-(1-ethenesulfonyl-piperidin-4-ylamino)-thiazol-5-yl]-(2,6-difluoro-phenyl)-methanone (Example F55; 250 mg, 0.74 mmol) and bis-cyclopropylmethylamine (278 mg, 2.22 mmol, see A. Donetti, et al; J. Org. Chem., Vol. 37, pp 3352-3353 (1972)) gave 235 mg of white powder in 57% yield.

¹H NMR: (DMSO-d₆): 8.68 (bs, 1H), 7.80 (bs, 2H), 7.41 (m, 1H), 7.08 (dd, 2H, J=7.7, 7.9 Hz), 3.45 (d, 2H, J=12.6 Hz), 2.30 (d, 4H, J=6.8 Hz), 0.36 (dd, 4H, J=4.4, 6.8 Hz), −0.01 (d, 4H, J=4.7 Hz). Anal. Calcd. for C₂₅H₃₃F₂N₅O₃S₂: C, 54.23; H, 6.01; N, 12.65; S, 11.58. Found C, 53.93; H, 5.98; N, 12.58; S, 11.29.

### Example K55

### {4-Amino-2-[1-(3-cyclohexylamino-propane-1-sulfonyl)-piperidin-4-ylamino]-thiazol-5-yl}-(2,6-difluoro-phenyl)-methanone

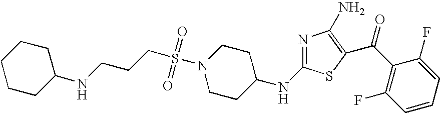

The title compound was prepared as follows. To a solution of 1-{4-amino-2-[1-(3-iodopropane-1-sulfonyl)-piperidin-4-ylamino]-thiazol-5-yl}-1-(2,6-difluoro-phenyl)-methanone (Example F45; 300 mg, 0.526 mmol) in dioxane (3 mL) were added cyclohexyamine (0.30 mL, 2.63 mmol). The mixture stirred at 100° C. for 4 hours, then was diluted with ether and hexane (25 ml, 1:1) and stirred rapidly for half hour, filtered, the yellow solid was washed with ether, dried over vacuum to provide 75 mg of product as a pale yellow powder in 91% yield.

¹H NMR (DMSO-d6): 68.76 (bs, 1H), 8.13 (bs, 2H), 7.56 (m, 1H), 7.23 (t, 2H, J=7.8 Hz), 3.59 (d, 2H, J=12.4 Hz), 3.12 (dd, 2H, J=7.1, 8.1 Hz), 2.99 (dd, 2H, J=10.6, 11.2 Hz), 2.37 (m, 1H). Anal. Calcd. for C₂₄H₃₃F₂N₅O₃S₂.0.2 H₂O.0.2 hexane: C, 53.81; H, 6.49; N, 12.45. Found: C, 53.98; H, 6.49; N, 12.09. ESMS (M+H): 542.10

### Example K56

### (4-Amino-2-{1-[3-(pyridin-2-ylamino)-propane-1-sulfonyl]-piperidin-4-ylamino}-thiazol-5-yl)-(2,6-difluoro-phenyl)-methanone. Trifluoroacetic Acid Salt

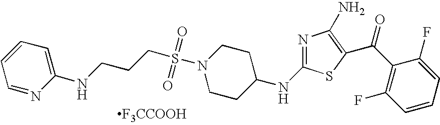

The title compound was prepared as follows. To a solution of 1-{4-amino-2-[1-(3-iodopropane-1-sulfonyl)-piperidin-4-ylamino]-thiazol-5-yl}-1-(2,6-difluoro-phenyl)-methanone (Example F45; 300 mg, 0.526 mmol) in dioxane (3 mL) were added 2-aminopyridine (248 mg, 2.63 mmol). The mixture heated at 120° C. for half hour in microwave, HPLC show 25% of conversion of starting material. Another 248 mg of 2-amino-pyridine was added, the mixture was heated at 120° C. (30 min.×3 times) until the reaction was completed by checking HPLC, cooled, then was diluted with ether and hexane (25 ml, 1:1) and stirred rapidly for half hour, filtered, the yellow solid was washed with ether, further purified by Preparative HPLC, obtained 210 mg of product as a pale yellow powder in 74% yield.

¹H NMR (DMSO-d₆): δ8.66 (bs, 1H), 8.36 (bs, 2H), 7.90 (d, 2H, J=7.0 Hz), 7.75 (dd, 2H, J=7.2, 7.7 Hz), 7.36 (m, 1H), 7.04 (dd, 2H, J=7.4, 8.3 Hz), 6.92 (d, 1H, J=8.7 Hz), 6.79 (dd,1H, J=5.7, 7.4 Hz). Anal. Calcd. for C₂₃H₂₆F₂N₆O₃S₂.2.6 CF₃COOH: C, 40.93; H, 3.50; N, 10.23: S, 7.80. Found: C, 41.00; H, 3.67; N, 10.41; S, 7.96. ESMS(M +H): 537.10.

### Example K57

### {2-[1-(3-Allylamino-propane-1-sulfonyl)-piperidin-4-ylamino]-4-amino-thiazol-5-yl}-(2,6-difluoro-phenyl)-methanone. Trifluoroacetic Acid Salt

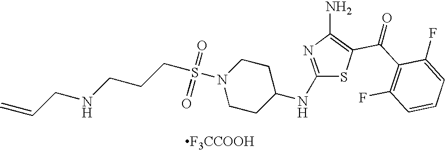

The title compound was prepared in a manner analogous to Example K56. 1-{4-amino-2-[1-(3-iodopropane-1-sulfonyl)-piperidin-4-ylamino]-thiazol-5-yl}-1-(2,6-difluoro-phenyl)-methanone (Example F45; 300 mg, 0.526 mmol) and allylamine (118 μL, 1.58 mmol). The mixture heated at 120° C. for 30 min. in microwave, purified by Preparative HPLC, obtained 205 mg of product as a white powder in 78% yield.

¹H NMR (DMSO-d₆): δ9.04 (bs, 1H), 8.86 (bs, 2H), 7.73 (m, 1H), 7.39 (dd, 2H, J=7.3, 8.3 Hz), 6.06 (m, 1H), 5.67 (dd, 2H, J=10.4, 19.0 Hz), 3.39 (dd, 2H, J=6.2, 7.7 Hz), 1.71 (q, 2H, J=11.5 Hz). Anal. Calcd. for C₂₁H₂₇F₂N₅O₃S₂.1.8 CF₃COOH: C, 41.92; H, 4.12; N, 9.94; S, 9.10. Found: C, 41.89; H, 4.11; N, 9.94; S, 9.05. ESMS(M+H): 500.10.

### Example K58

### {4-Amino-2-[(1-{[3-(4-methylpiperidin-1-yl)propyl]sulfonyl}piperidin-4-yl)amino]-1,3-thiazol-5-yl}(2,6-difluorophenyl)methanone

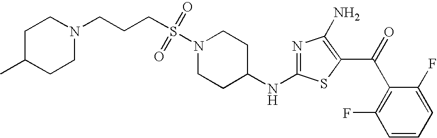

The title compound was prepared in a manner analogous to Example K56. 1-{4-amino-2-[1-(3-iodopropane-1-sulfonyl)-piperidin-4-ylamino]-thiazol-5-yl}-1-(2,6-difluoro-phenyl)-methanone (Example F45; 400 mg, 0.701 mmol) and 4-methylpiperidine (209 mg, 2.10 mmol) gave a crude residue that was purified by silica gel chromatography (eluting with 2.5-10% methanol in dichloromethane) to afford 200 mg of a yellow powder in 51% yield.

¹H NMR (CD₃OD): 7.35 (m, 1H), 6.93 (t, J=7.82 Hz, 2H), 3.61 (m, 2H), 3.21 (m, 3H), 2.95 (m, 2H), 2.85 (m, 2H), 2.38 (m, 2H), 2.06-1.81 (m, 6H), 1.53 (m, 4H), 1.30 (m, 1H), 1.14 (m, 2H), 0.86, 0.83 (s, 3H). Anal. Calcd. for C₂₄H₃₃F₂N₅O₃S₂: C, 53.22; H, 6.14; N, 12.93; S, 11.84; F, 7.01. Found C, 53.07; H, 6.28; N, 12.91; S, 11.73; F, 6.80.

### Example K59

### {4-Amino-2-[(1-{[3-(4-methoxypiperidin-1-yl)propyl]sulfonyl}piperidin-4-yl)amino]-1,3-thiazol-5-yl}(2,6-difluorophenyl)methanone

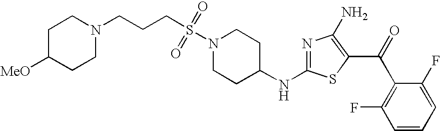

The title compound was prepared in a manner analogous to Example K56. 1-{4-amino-2-[1-(3-iodopropane-1-sulfonyl)-piperidin-4-ylamino]-thiazol-5-yl}-1-(2,6-difluoro-phenyl)-methanone (Example F45; 400 mg, 0.701 mmol), 4-methoxypiperidine (200 mg, 1.74 mmol), and N,N-diisopropylethylamine (122 μL, 0.701 mmol) gave a crude residue that was triturated from ethyl acetate to afford 242 mg of a pale yellow powder in 62% yield.

¹H NMR (DMSO-d6): 8.06 (bs, 2H), 7.49 (m, 1H), 7.16 (t, J=7.82 Hz, 2H), 3.51 (m, 2H), 3.20 (s, 3H), 3.13 (m, 2H), 3.00 (m, 2H), 3.01 (m, 2H), 2.60 (m, 2H), 2.32 (t, J=6.88 Hz, 2H), 1.98 (m, 4H), 1.78 (m, 4H), 1.55-1.30 (m, 4H). Anal. Calcd. for C₂₄H₃₃F₂N₅O₄S₂.0.05(CH₂Cl₂): C, 51.40; H, 5.94; N, 12.46; S, 11.41; F, 6.76. Found C, 51.50; H, 6.00; N, 12.49; S, 11.41; F, 6.76.

### Example K60

### {4-Amino-2-[(1-{[3-(3,3-dimethylpiperidin-1-yl)propyl]sulfonyl}piperidin-4-yl)amino]-1,3-thiazol-5-yl}(2,6-difluorophenyl)methanone

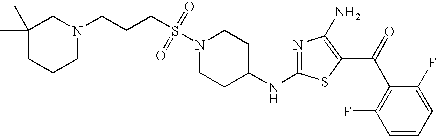

The title compound was prepared as follows. To a suspension of 1-{4-amino-2-[1-(3-iodopropane-1-sulfonyl)-piperidin-4-ylamino]-thiazol-5-yl}-1-(2,6-difluoro-phenyl)-methanone (Example F45; 400 mg, 0.701 mmol) in DMSO (2.5 mL) was added 3,3-dimethylpiperidine (237 mg, 2.10 mmol). The mixture was placed in a microwave reactor at 120° C. for 15 min, then partitioned between EtOAc (100 mL) and H₂O (100 mL). The organic layer was dried over Na₂SO₄ and concentrated in vacuo. Silica gel chromatography (eluting with 2.5-10% methanol in dichloromethane) of the crude residue afforded 200 mg of a yellow powder in 50% yield.

¹H NMR (CD₃OD): 7.43 (m, 1H), 7.01 (m, 2H), 3.69 (m, 2H), 3.30 (m, 3H), 3.07 (m, 2H), 2.98 (m, 2H), 2.37 (m, 4H), 2.05 (m, 4H), 1.91 (m, 2H), 1.66-1.51 (m, 4H), 1.24 (m, 2H), 0.94 (s, 6H). Anal. Calcd. for C₂₅H₃₅F₂N₅O₃S₂.0.15 DMSO: C, 53.55; H, 6.38; N, 12.30; S, 12.15; F, 6.70. Found C, 53.24; H, 6.44; N, 12.30; S, 12.09; F, 6.61.

### Example K61

### (4-Amino-2-{1-[3-(cyclohexyl-methyl-amino)-propane-1-sulfonyl]-piperidin-4-ylamino}-thiazol-5-yl)-(2,6-difluoro-phenyl)-methanone. Acetic Acid Salt

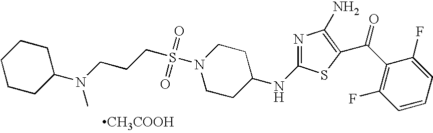

The title compound was prepared as follows. To a solution of 1-{4-amino-2-[1-(3-iodopropane-1-sulfonyl)-piperidin-4-ylamino]-thiazol-5-yl}-1-(2,6-difluoro-phenyl)-methanone (Example F45; 456 mg, 0.80 mmol) in DMSO (4 mL) were added N-methyl cyclohexyamine (313 μL, 2.4 mmol). The mixture stirred at 100° C. for overnight, the mixture was extracted with ethyl acetate, the organic layer was dried over Na₂SO₄, concentrated. The residue was purified by Preparative HPLC to provide the title compound as a white powder in 45% yield.

¹H NMR (DMSO-d₆): δ 8.72 (bs, 1H), 7.99 (bs, 2H), 7.43 (m, 1H), 7.10 (dd, 2H, J=7.5, 8.3 Hz), 3.45 (d, 2H, J=12.4 Hz), 2.93 (dd, 2H, J=7.5, 8.0 Hz), 2.84 (d, 2H, J=12.4 Hz), 2.38 (dd, 2H, J=6.6, 7.0 Hz), 2.20 (dd, 1H, J=8.9, 11.7Hz), 2.09 (s, 3H). Anal. Calcd. for C₂₅H₃₅F₂N₆O₂S₂.0.3 CH₃COOH.1.0 H₂O: C, 51.96; H, 6.51; N, 11.84; S, 10.84. Found: C, 52.30; H, 6.45; N, 11.72; S, 10.76. ESMS(M+H): 556.15.
Method L:

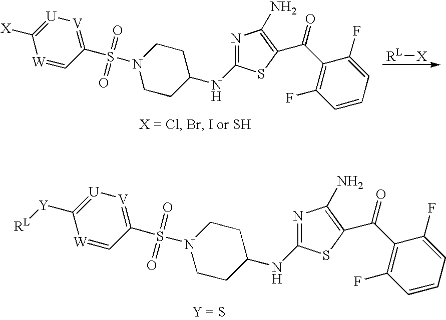

### Example L1

### 1-(4-Amino-2-{1-[6-(2-dimethylamino-ethylsulfanyl)-pyridine-3-sulfonyl]-piperidin-4-ylamino}-thiazol-5-yl)-1-(2,6-difluoro-phenyl)-methanone Dihydrochloride.

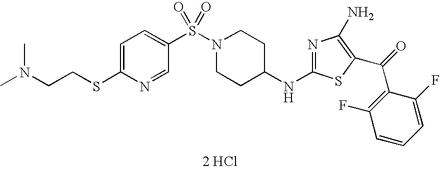

A solution of 1-{4-amino-2-[1-(6-chloro-pyridine-3-sulfonyl)-piperidin-4-ylamino]-thiazol-5-yl}-1-phenyl-methanone (Example F21; 100 mg, 0.195 mmol), 2-dimethylamino-ethanethiol hydrochloride (150 mg, 1.42 mmol), and potassium tert-butoxide (200 mg, 1.63 mmol) in DMSO (10 ml) stirred for 16 hours at room temperature. The mixture was diluted with EtOAc, washed with sat. NaHCO₃, dried over MgSO₄, filtered, and concentrated. Column chromatography (58% NH₄OH/MeOH/EtOAc=1/5/44) afforded a yellow solid, which was dissolved in EtOAc, washed with sat. NaHCO₃, dried over MgSO₄, filtered, concentrated, and dissolved in 30% CH₃CN/H₂O (200 ml). Conc. HCl (2 ml) was added and lyophilization gave 68 mg of an off-white powder in 49% yield.

¹H NMR (CD₃OD): δ 8.75 (d, 2H, J=2.4 Hz), 7.88 (dd, 1H, J=2.4, 8.5 Hz), 7.57-7.41 (m, 2H), 7.12-7.00 (m, 2H), 3.68-3.49 (m, 4H), 3.48-3.34 (m, 3H), 2.90 (s, 6H), 2.69-2.52 (m, 2H), 2.08-1.96 (m, 2H), 1.68-1.53 (m, 2H). ESIMS (MH⁺): 583. Anal. Calcd for C₂₄H₂₈F₂N₆O₃S₃.3.0 HCl.2.0 H₂O: C, 39.59; H, 4.85; N, 11.54; S, 13.21. Found: C, 39.31; H, 5.18; N, 11.70; S, 13.16.

### Example L2

### 1-(4-Amino-2-{1-[6-(pyridin-2-ylsulfanyl)-pyridine-3-sulfonyl]-piperidin-4-ylamino}-thiazol-5-yl)-1-(2,6-difluoro-phenyl)-methanone Dihydrochloride.

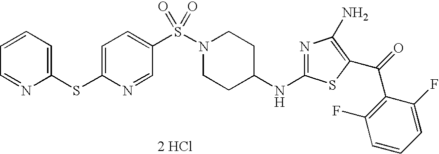

The title compound was prepared in a manner similar to that for Example L1 from 1-{4-amino-2-[1-(6-chloro-pyridine-3-sulfonyl)-piperidin-4-ylamino]-thiazol-5-yl}-phenyl-methanone (Example F21) and 2-mercaptopyridine.

¹H NMR (CD₃OD): δ 8.87-8.74 (m, 2H), 8.37 (m, 1H), 8.19-8.06 (m, 2H), 7.87 (m, 1H), 7.70 (m, 1H), 7.59 (m, 1H), 7.20-7.08 (m, 2H), 3.73-3.62 (m, 3H), 2.76-2.63 (m, 2H), 2.14-2.00 (m, 2H), 1.73-1.59 (m, 2H). ESIMS (MH⁻): 587. Anal. Calcd for C₂₅H₂₂F₂N₆O₃S₃.2.0 HCl.1.0 H₂O: C, 44.18; H, 3.86; N, 12.37; S, 14.15. Found: C, 44.08; H, 4.03; N, 12.33; S, 14.21.

### Example L3

### 1-(4-Amino-2-{1-[6-(2-pyridin-2-yl-ethylsulfanyl)-pyridine-3-sulfonyl]-piperidin-4-ylamino}-thiazol-5-yl)-1-(2,6-difluoro-phenyl)-methanone Hydrochloride.

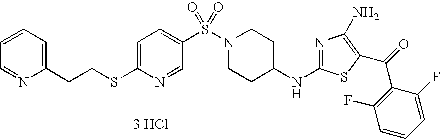

The title compound was prepared in a manner similar for Example L1 from 1-{4-amino-2-[1-(6-chloro-pyridine-3-sulfonyl)-piperidin-4-ylamino]-thiazol-5-yl}-1-phenyl-methanone (Example F21) and 2-pyridylethylmercaptan (Toronto Research Chemicals).

¹H NMR (CD₃OD): δ 8.78-8.64 (m, 2H), 8.53 (m, 1H), 8.10 (d, 1H, J=8.6 Hz), 7.97-7.83 (m, 2H), 7.59 (m, 1H), 7.44 (d, 1H, J=8.1), 7.19-7.08 (m, 2H), 3.80-3.63 (m, 4H), 3.62-3.52 (m, 3H), 2.72-2.60 (m, 2H), 2.17-2.06 (m, 2H), 1.73-1.60 (m, 2H). ESIMS (MH⁺): 617. Anal. Calcd for C₂₇H₂₆F₂N₆O₃S₃.3.0 HCl.1.0 H₂O: C, 43.58; H, 4.20; N, 11.29; S, 12.93. Found: C, 43.23; H, 4.46; N, 11.24; S, 12.88.

### Example L4

### 1-{4-Amino-2-[1-(6-mercapto-pyridine-3-sulfonyl)-piperidin-4-ylamino]-thiazol-5-yl}-1-(2,6-difluoro-phenyl)-methanone Hydrochloride.

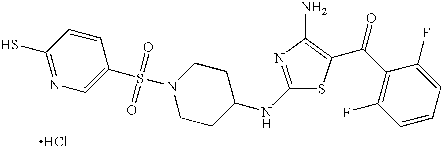

### 1-{4-Amino-2-[1-(6-mercapto-pyridine-3-sulfonyl)-piperidin-4-ylamino]-thiazol-5-yl}-1-(2,6-difluoro-phenyl)-methanone

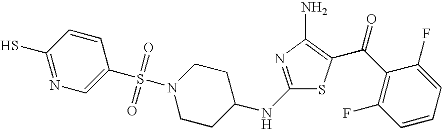

A solution of 1-{4-amino-2-[1-(6-chloro-pyridine-3-sulfonyl)-piperidin-4-ylamino]-thiazol-5-yl}-1-phenyl-methanone (Example F21; 415 mg, 0.809 mmol) and potassium hydrogen sulfide (490 mg, 6.80 mmol) in absolute ethanol (30 ml) was refluxed for 5 hours. The ethanol was distilled off. The residue was dissolved in EtOAc, washed with sat. NaHCO₃, dried over MgSO₄, filtered, and concentrated. The resultant solid was triturated with ether, filtered, rinsed, and dried to give 380 mg of a yellow solid in 92% yield, which was used without any further purification.

¹H NMR (CD₃OD): δ 7.96 (d, 1H, J=1.9 Hz), 7.55-7.37 (m, 3H), 7.06-6.95 (m, 2H), 3.72-3.57 (m, 3H), 2.82-2.70 (m, 2H), 2.17-2.01 (m, 2H), 1.70-1.54 (m, 2H).

The title compound was prepared as follows. A small portion of 1-{4-amino-2-[1-(6-mercapto-pyridine-3-sulfonyl)-piperidin-4-ylamino]-thiazol-5-yl}-1-(2,6-difluoro-phenyl)-methanone was purified via preparative HPLC, the fractions were treated with HCl, and lyophilized to obtain a yellow solid.

¹H NMR (CD₃OD): δ 7.96 (d, 1H, J=2.6 Hz), 7.57-7.42 (m, 3H), 7.10-7.00 (m, 2H), 3.72-3.58 (m, 3H), 2.83-2.70 (m, 2H), 2.17-2.03 (m, 2H), 1.72-1.53 (m, 2H). ESIMS (MH⁺): 512. Anal. Calcd. for C₂₀H₁₉F₂N₅O₃S₃.0.5 HCl.0.25 H₂O.0.5 CH₃CN: C, 45.46; H, 3.91; N, 13.88; S, 17.34. Found; C, 45.73; H, 3.92; N, 13.78; S, 17.54.

### Example L5

### 1-(4-Amino-2-{1-[6-(3-dimethylamino-propylsulfanyl)-pyridine-3-sulfonyl]-piperidin-4-ylamino}-thiazol-5-yl)-1-(2,6-difluoro-phenyl)-methanone Dihydrochloride.

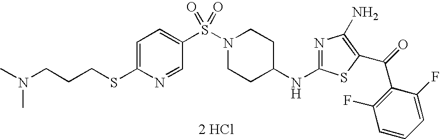

A solution of 1-{4-amino-2-[1-(6-mercapto-pyridine-3-sulfonyl)-piperidin-4-ylamino]-thiazol-5-yl}-1-(2,6-difluoro-phenyl)-methanone (Example L4; 75 mg, 0.15 mmol), 3-dimethylaminopropyl chloride hydrochloride (160 mg, 1.01 mmol), and N,N-diisopropylethylamine (327 ul, 1.88 mmol) in DMF (5 ml) stirred at room temperature for 16 hours. The mixture was diluted with EtOAc, washed with sat. NaHCO₃, dried over MgSO₄, filtered, and concentrated. Preparative HPLC afforded 42 mg of yellow solid in 48% yield.

¹H NMR (CD₃OD): δ 8.78 (m, 1H), 7.90 (m, 1H), 7.49-7.40 (m, 2H), 7.08-6.97 (m, 2H), 3.72-3.61 (m, 3H), 3.40-3.21 (m, 4H), 2.90 (s, 6H), 2.69-2.60 (m, 2H), 2.26-2.00 (m, 4H), 1.70-1.53 (m, 2H). ESIMS (MH⁺): 597. Anal. Calcd for C₂₅H₃₀F₂N₆O₃S₃.2.2 HCl.1.0 H₂O: C, 43.20; H, 4.96; N, 12.09; S, 13.84. Found: C, 43.18; H, 5.00; N, 12.02; S, 13.85.

### Example L6

### 1-[4-Amino-2-(1-{6-[2-(1-methyl-pyrrolidin-2-yl)-ethylsulfanyl]-pyridine-3-sulfonyl}-piperidin-4-ylamino)-thiazol-5-yl]-1-(2,6-difluoro-phenyl)-methanone Dihydrochloride.

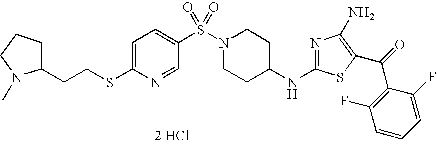

The title compound was prepared in a manner similar to that for Example L5 from 1-{4-amino-2-[1-(6-mercapto-pyridine-3-sulfonyl)-piperidin-4-ylamino]-thiazol-5-yl}-1-(2,6-difluoro-phenyl)-methanone (Example L4) and 2-(2-chloroethyl)-1-methylpyrrolidine hydrochloride.

¹H NMR (CD₃OD): δ 8.78 (d, 1H, J=2.4 Hz), 7.91 (dd, 1H, J=2.4, 8.5 Hz), 7.52-7.39 (m, 2H), 7.08-6.97 (m, 2H), 3.78-3.62 (m, 4H), 3.51-3.40 (m, 3H), 3.30-3.12 (m, 2H), 2.94 (s, 3H), 2.70-2.65 (m, 2H), 2.57-2.30 (m, 2H), 2.20-1.83 (m, 5H), 1.71-1.53 (m, 2H). ESIMS (MH⁺): 623. Anal. Calcd for C₂₇H₃₂F₂N₆O₃S₃.2.0 HCl.1.0 H₂O: C, 45.44; H, 5.08; N, 11.78; S, 13.48. Found: C, 45.52; H, 5.15; N, 11.82; S, 13.41.

### Example L7

### 1-(4-Amino-2-{1-[6-(2-morpholin-4-yl-ethylsulfanyl)-pyridine-3-sulfonyl]-piperidin-4-ylamino}-thiazol-5-yl)-1-(2,6-difluoro-phenyl)-methanone Dihydrochloride.

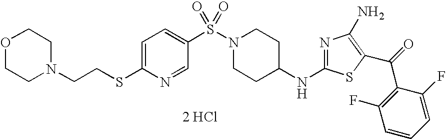

The title compound was prepared in a manner similar to that for Example L5 from 1-{4-amino-2-[1-(6-mercapto-pyridine-3-sulfonyl)-piperidin-4-ylamino]-thiazol-5-yl}-1-(2,6-difluoro-phenyl)-methanone (Example L4) and 4-(2-chloroethyl)morpholine hydrochloride.

¹H NMR (CD₃OD): δ 8.83 (m, 1H), 7.96 (m, 1H), 7.59-7.44 (m, 2H), 7.12-7.03 (m, 2H), 4.14-4.03 (m, 3H), 3.89-3.48 (m, 12H), 2.78-2.60 (m, 2H), 2.18-2.00 (m, 2H), 1.77-1.57 (m, 2H). ESIMS (MH⁺): 625. Anal. Calcd for C₂₆H₃₀F₂N₆O₄S₃.2.0 HCl.1.0 H₂O: C, 45.44; H, 5.08; N, 11.78; S, 13.48. Found: C, 45.52; H, 5.15; N, 11.82; S, 13.41.
Method M:

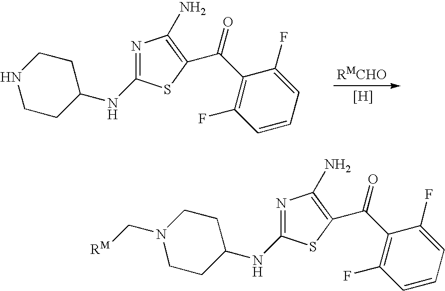

### Example M1

### 1-[4-Amino-2-(1-pyridin-2-ylmethyl-piperidin-4-ylamino)-thiazol-5-yl]-1-(2,6-difluoro-phenyl)-methanone

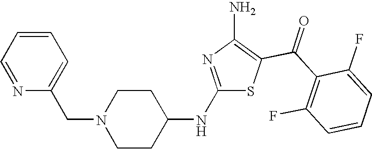

1-[4-Amino-2-(piperidine-4-ylamino)-thiazol-5-yl]-1-(2,6-difluoro-phenyl)-methanone (Example A6; 380 mg, 1,12 mmol) was dissolved in 10 ml ethanol (10 ml). Pyridine-2-carboxaldehyde (1.50 g, 14.0 mmol) was added and stirred for 2.5 hr. Sodium cyanoborohydride (1.00 g, 15.9 mmol) was added and the reaction was stirred overnight. The mixture was poured into water and then extracted with ethyl acetate. Organic layer was dried and evaporated. The residue was purified via flash column (10% methanol/methylene chloride) to yield 300 mg of solid in 62% yield.

¹H NMR (DMSO d₆): δ 8.78 (bs, 1H), 8.72-8.67 (bs, 1H),8.05 (bs, 2H), 7.53-7.41 (m, 2H), 7.38-7.24 (m, 1H), 7.17-7.12 (m, 2H), 3.76 (m, 2H), 2.76 (m, 2H), 2.26 (m, 2H), 2.07 (m, 2H), 1.55-1.46 (m, 2H). Anal. Calcd for C₂₁H₂₁F₂N₅OS.0.15 Et₂O: C, 58.82; H, 4.80; N, 15.88. Found: C, 58.57; H, 5.28; N, 15.57.

### Example M2

### 1-[4-Amino-2-(1-pyridin-4-ylmethyl-piperidin-4-ylamino)-thiazol-5-yl]-1-(2,6-difluoro-phenyl)-methanone.

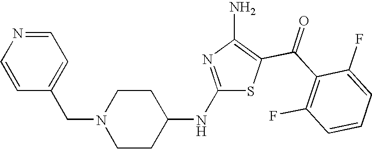

The title compound was prepared in a manner similar to that of Example M1.

¹H NMR (DMSO d₆): δ 8.49 (d, J=5.8 Hz, 2H), 8.2 (bs, 1H), 7.53-7.41 (m, 1H), 7.30-7.22 (m, 3H), 7.17-7.12 (m, 2H), 4.5 (d, J=5.7Hz, 2H), 3.47(bs, 2H), 2.74-2.70 (m, 2H), 2.26 (m, 2H), 2.08-2.00 (m, 2H), 1.55-1.46 (m, 2H). Anal. Calcd for C₂₁H₂₁F₂N₅OS.0.25 Et₂O: C, 58.94; H, 5.24; N, 15.62. Found: C, 59.34; H, 5.28; N, 15.39.
Method N:

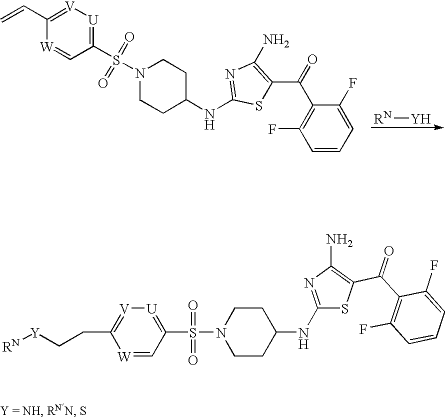

### Example N1

### [4-Amino-2-(1-{6-[2-(2-hydroxy-phenylamino)-ethyl]-pyridine-3-sulfonyl}-piperidin-4-ylamino)-thiazol-5-yl]-(2,6-difluoro-phenyl)-methanone Trifluoroacetic Acid Salt.

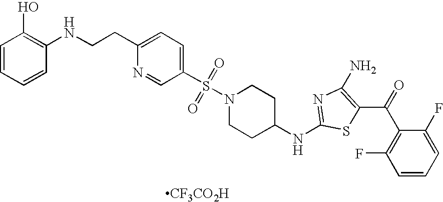

The title compound was made as follows. Based on a procedure from Winn, et al.; J. Med. Chem.; 39; 1039-1048 (1996), 2-amino-1-hydroxybenzene (310 mg, 2.84 mmol) and acetic acid (2 drops) were added in succession to a solution of {4-amino-2-[1-(6-vinyl-pyridine-3-sulfonyl)-piperidin-4-ylamino]-thiazol-5-yl}-(2,6-difluoro-phenyl)-methanone (Example I15; 100 mg, 0.198 mmol) in methoxyethanol (1 ml). The mixture was stirred at 100° C. for 4 hours, solvent evaporated, and purified via preparative HPLC to obtain 72 mg of a yellow solid in 59% yield.

¹H NMR (DMSO-d₆): δ 8.82 (s, 1H), 8.08 (d, 1H, =8.9 Hz), 8.01 (bs, 2H), 7.61(d, 1H, J=8.3 Hz), 7.47 (m, 1H), 7.14 (dd, 2H, J=7.6, 8.1 Hz), 6.93 (bs, 1H), 3.60 (dd, 2H, J=6.8, 7.3 Hz), 3.51 (dd, 2H, J=12.3 Hz), 3.20 (dd, 2H, J=6.8, 7.2 Hz). HRESIMS. Calcd for C₂₈H₂₉F₂N₆O₄S₂ (M+H³⁰ ): 615.1660. Found: 615.1650. Anal. Calcd. for C₂₈H₂₈F₂N₆O₄S₂.2.8 TFA: C, 43.21; H, 3.32; N, 9.00; S, 6.87. Found: C, 43.35; H, 3.55; N, 9.14; S, 7.02.

### Example N2

### (4-Amino-2-{1-[6-(2-pyrrolidin-1-yl-ethyl)-pyridine-3-sulfonyl]-piperidin-4-ylamino}-thiazol-5-yl)-(2,6-difluoro-phenyl)-methanone Hydrochloride Salt.

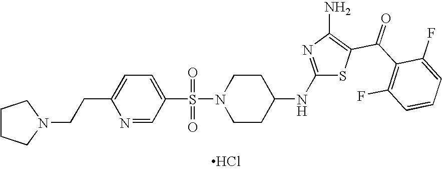

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I55; 90 mg, 0.18 mmol) and pyrrolidine (38 mg, 0.53 mmol) and subsequent hydrochloride salt formation gave 74 mg of white powder in 72% yield.

¹H NMR (DMSO-d₆): δ 10.73 (bs, 1H), 8.83 (bs, 1H), 8.82 (s, 1H), 8.12 (d, 1H, J=6.4 Hz), 8.05 (bs, 1H), 7.65 (d, 1H, J=7.7 Hz), 7.48 (t, 1H, J=6.4 Hz), 7.15 (d, 1H, J=7.1 Hz). HRESIMS. Calcd for C₂₆H₃₁F₂N₆O₃S₂ (M+H³⁰ ): 577.1867. Found: 577.1872. Anal. Calcd. for C₂₆H₃₀F₂N₆O₃S₂.1.5 H₂O.3.0 HCl: C, 43.79; H, 5.09; N, 11.79; S, 8.99. Found: C, 43,47; H, 5.20; N, 11.67; S, 9.30.

### Example N3

### (4-Amino-2-{1-[6-(2-morpholin-4-yl-ethyl)-pyridine-3-sulfonyl]-piperidin-4-ylamino}-thiazol-5-yl)-(2,6-difluoro-phenyl)-methanone Hydrochloride Salt.

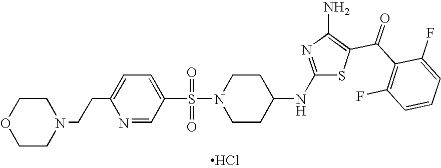

The title compound was prepared in a manner analogous to Example N1. {4-Amino-2-[1-(6-vinyl1-pyridine-3-sulfonyl)-piperidin-4-ylamino]-thiazol-5-yl}-(2,6-difluoro-phenyl)-methanone (Example I15; 90 mg, 0.18 mmol) and morpholine (46 mg, 0.53 mmol) and subsequent hydrochloride salt formation gave 69 mg of white powder in 65% yield.

¹H NMR (DMSO-d₆): δ 11.52 (bs, 1H), 8.99 (bs, 1H), 8.82 (s, 1H), 8.12 (dd, 1H, J=1.7, 8.1 Hz), 7.64 (d, 1H, J=8.1 Hz), 7.48 (m, 1H), 7.14 (dd, 2H, J=7.7, 8.0 Hz), 2.72 (m, 1H). HRESIMS. Calcd for C₂₆H₃₁F₂N₆O₄S₂ (M+H³⁰ ): 593.1816. Found: 593.1827. Anal. Calcd. for C₂₆H₃₀F₂N₆O₄S₂.2.0 H₂O.3.0 HCl: C, 42.31; H, 5.05; N, 11.39; S, 8.69. Found: C, 42,28; H, 5.28; N, 11.41; S, 8.91.

### Example N4

### [4-Amino-2-(1-{6-[2-(4-methyl-piperazin-1-yl)-ethyl]-pyridine-3-sulfonyl}-piperidin-4-ylamino)-thiazol-5-yl]-(2,6-difluoro-phenyl)-methanone Hydrochloride Salt.

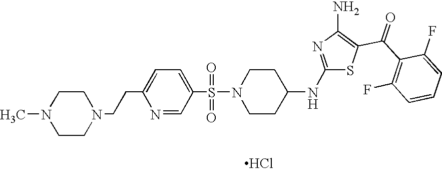

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I15; 90 mg, 0.18 mmol) and N-methyl-piperazine (53 mg, 0.53 mmol) and subsequent hydrochloride salt formation gave 72 mg of white amorphous solid in 67% yield.

¹H NMR (DMSO-d₆): δ 11.98 (bs, 1H), 9.00 (bs, 1H), 8.82 (s, 1H), 8.13 (d, 1H, J=8.3 Hz), 7.66 (d, 1H, J=8.3 Hz), 7.48 (m, 1H), 7.15 (dd, 2H, J=7.7, 8.0 Hz), 2.82(s, 3H). HRESIMS. Calcd for C₂₇H₃₄F₂N₇O₃S₂ (M+H³⁰ ): 606.2133. Found: 606.2137. Anal. Calcd. for C₂₇H₃₃F₂N₇O₃S₂.3.0 H₂O.4.0 HCl: C, 40.25; H, 5.38; N, 12.17; S, 7.96. Found: C, 40.39; H, 5.55; N, 12.02; S, 8.06.

### Example N5

### (4-Amino-2-{1-[2-(3-phenyl-pyrrolidin-1-yl)-ethanesulfonyl]-piperidin-4-ylamino}-thiazol-5-yl)-(2,6-difluoro-phenyl)-methanone Hydrochloride Salt.

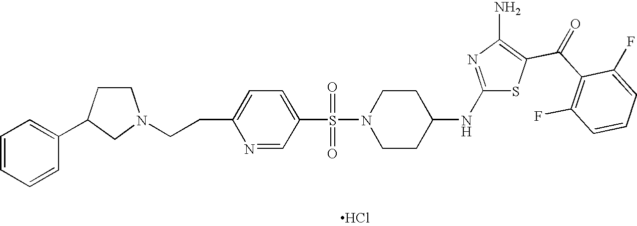

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I15; 90 mg, 0.18 mmol) and 3-phenyl-pyrrolidine (from Example F24; 90 mg, 0.18 mmol) and subsequent hydrochloride salt formation gave 73 mg of white powder in 71% yield.

¹H NMR (DMSO-d₆): δ 11.38 (bs, 1H), 9.01 (bs, 1H), 8.14 (s, 1H), 7.57 (m, 1H), 7.24 (dd, 2H, J=7.7, 8.0 Hz), 3.11 (dd, 2H, J=10.9, 11.1 Hz). HRESIMS. Calcd for C₂₇H₃₂F₂N₅O₃S₂ (M+H³⁰ ): 576.1975. Found: 576.1942. Anal. Calcd. for C₂₇H₃₁F₂N₅O₃S₂.0.2 hexane.3.0 HCl: C, 48.23; H, 5.28; N, 9.97; S, 9.13. Found: C, 48.60; H, 5.29; N, 10.07; S, 9.05.

### Example N6

### [4-Amino-2-(1-{6-[2-(3-hydroxy-phenylamino)-ethyl]-pyridine-3-sulfonyl}-piperidin-4-ylamino)-thiazol-5-yl]-(2,6-difluoro-phenyl)-methanone Hydrochloride Salt.

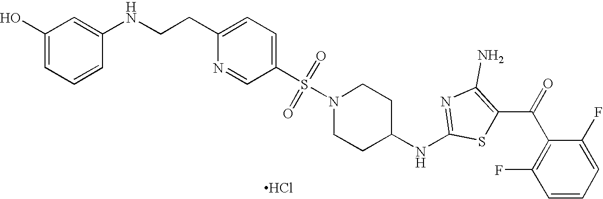

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I15; 90 mg, 0.18 mmol) and 3-aminophenol (100 mg, 0.53 mmol) and subsequent hydrochloride salt formation gave 88 mg of white powder in 72% yield.

¹H NMR (DMSO-d₆): δ 8.92 (bs, 1H), 8.84 (s, 1H), 8.15 (bs, 1H), 8.10 (d, 1H, J=6.6 Hz), 7.69 (d, 1H, J=8.2 Hz), 7.49 (m, 1H), 7.27 (dd, 1H, J=8.0, 8.0 Hz), 7.16 (dd, 1H, J=7.7, 8.0 Hz), 6.72 (dd, 2H, J=1.6, 6.6 Hz), 3.68 (dd, 2H, J=7.2, 7.4 Hz), 3.32 (dd, 2H, J=7.2, 7.2 Hz). HRESIMS. Calcd for C₂₈H₂₉F₂N₆O₄S₂ (M+H³⁰ ): 615.1660. Found: 615.1668. Anal. Calcd. for C₂₈H₂₈F₂N₆O₄S₂.3.8 HCl: C, 44.65; H, 4.26; N, 11.16; S, 8.51. Found: C, 44.72; H, 4.35; N, 10.92; S, 8.41.

### Example N7

### [4-Amino-2-(1-{6-[2-(3-hydroxy-pyrrolidin-1-yl)-ethyl]-pyridine-3-sulfonyl}-piperidin-4-ylamino)-thiazol-5-yl]-(2,6-difluoro-phenyl)-methanone Hydrochloride Salt.

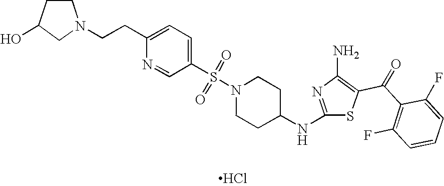

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I15; 90 mg, 0.18 mmol) and 3-pyrrolidinol (46 mg, 0.53 mmol) and subsequent hydrochloride salt formation gave 70 mg of white powder in 66% yield.

¹H NMR (DMSO-d₆): δ 11.17 (bs, 1H), 10.74 (s, 1H), 9.03 (bs, 1H), 8.82 (s, 1H), 8.12 (bs, 2H), 7.65 (dd, 2H, J=3.3, 8.1 Hz), 7.48 (m, 1H), 7.14 (dd, 2H, J=7.8, 7.9 Hz), 4.44 (s, 1H), 4.38 (s, 1H), 3.02 (d, 1H, J=11.7 Hz), 2.25 (m, 1H). HRESIMS. Calcd for C₂₆H₃₁F₂N₆O₄S₂(M+H³⁰ ): 593.1816. Found: 593.1836. Anal. Calcd. for C₂₆H₃₀F₂N₆O₃S₂.2.0 H₂O.3.5 HCl: C, 41.29; H, 5.00; N, 11.11; S, 8.48. Found: C, 41.37; H, 5.03; N, 11.23; S, 8.41.

### Example N8

### [4-Amino-2-(1-{6-[2-cis-3,5-dimethyl-piperazin-1-yl)-ethyl]-pyridine-3-sulfonyl}-piperidin-4-ylamino)-thiazol-5-yl]-(2,6-difluoro-phenyl)-methanone Hydrochloride Salt.

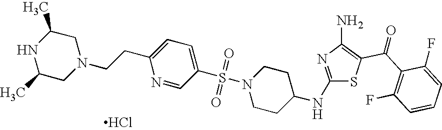

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I15; 100 mg, 0.199 mmol) and cis-2,6-dimethylpiperazine (68 mg, 0.59 mmol) and subsequent hydrochloride salt formation gave 81 mg of white powder in 66% yield.

¹H NMR (DMSO-d₆): δ 11.36 (bs, 1H), 10.17 (bs, 1H), 8.99 (bs, 1H), 8.86 (s, 1H), 8.16 (d, 1H, J=8.3 Hz), 7.68 (d, 1H, J=8.3 Hz), 7.51 (m, 1H), 7.17 (dd, 2H, J=7.8, 8.0 Hz), 3.27 (dd, 2H, J=12.7, 12.8 Hz), 1.37 (d, 6H, J=6.3 Hz). HRESIMS. Calcd for C₂₈H₃₆F₂N₇O₃S₂ (M+H³⁰ ): 620.2289. Found: 620.2286. Anal. Calcd. for C₂₈H₃₅F₂N₇O₃S₂.2.0 H₂O 4.5 HCl: C, 41.02; H, 5.35; N, 11.96; S, 7.82. Found: C, 40.86; H, 5.48; N, 11.98; S, 7.72.

### Example N9

### [4-Amino-2-(1-{6-[2-(2S-hydroxymethyl-pyrrolidin-1-yl)-ethyl]-pyridine-3-sulfonyl}-piperidin-4-ylamino)-thiazol-5-yl]-(2,6-difluoro-phenyl)-methanone Hydrochloride Salt.

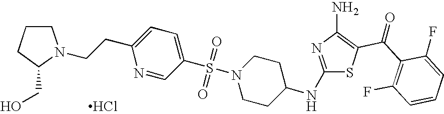

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I15; 90 mg, 0.18 mmol) and (S)-(+)-2-pyrrolidinemethanol (54 mg, 0.53 mmol) and subsequent hydrochloride salt formation gave 83 mg of white powder in 86% yield.

¹H NMR (DMSO-d₆): δ 10.29 (bs, 1H), 8.94 (bs, 1H), 8.83 (s, 1H), 8.13 (d, 1H, J=8.3 Hz), 8.08 (bs, 1H), 7.64 (d, 1H, J=8.3 Hz), 7.48 (m, 1H), 7.15 (dd, 2H, J=7.8, 8.0 Hz), 3.17 (m, 1H). HRESIMS. Calcd for C₂₇H₃₃F₂N₆O₄S₂ (M+H³⁰ ): 607.1973. Found: 607.1967. Anal. Calcd. for C₂₇H₃₂F₂N₆O₄S₂.4.0 HCl: C, 43.09; H, 4.82; N, 11.17; S, 8.52. Found: C, 43,05; H, 5.09; N, 11.03; S, 8.41.

### Example N10

### [4-Amino-2-(1-{6-[2-(1α, 5β, 6γ-amino-3-aza-bicyclo[3.1.0]hex-3-yl)-ethyl]-pyridine-3-sulfonyl}-piperidin-4-ylamino)-thiazol-5-yl]-(2,6-difluoro-phenyl)-methanone Hydrochloride Salt.

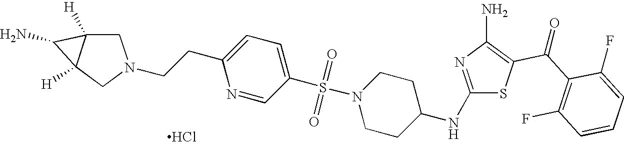

The title compound was prepared in a manner analogous to Example N1. {4-Amino-2-[1-(6-vinyl-pyridine-3-sulfonyl)-piperidin-4-ylamino]-thiazol-5-yl}-(2,6-difluoro-phenyl)-methanone (Example I15; 90 mg, 0.18 mmol) and (1R, 5S, 6S)-1,5-dimethyl-3-aza-bicyclo[3,1,0]hex-6-ylamine (79 mg, 0.53 mmol; Norris, et al., J. Chem. Soc. Perkin Trans. 1, 1615-1622 (2000)) and subsequent hydrochloride salt formation gave 79 mg of white powder in 73% yield.

¹H NMR (DMSO-d₆): δ 11.54 (bs, 1H), 8.87 (bs, 1H), 8.79 (s, 1H), 8.52 (s, 2H), 8.10 (d, 1H, J=8.2 Hz), 8.01 (bs, 1H), 7.58 (d, 1H, J=8.2 Hz), 7.46 (m, 1H), 7.13 (dd, 2H, J=7.7. 8.0 Hz), 2.62 (m, 1H). HRESIMS. Calcd for C₂₇H₃₂F₂N₇O₃S₂ (M+H³⁰ ): 604.1976. Found: 604.1978. Anal. Calcd. for C₂₇H₃₁F₂N₇O₃S₂.2.0 H₂O.3.5 HCl: C, 45.26; H, 5.06; N, 12.78; S, 8.36. Found: C, 41.99; H, 5.26; N, 12.90; S, 8.17.

### Example N11

### (4-Amino-2-{1-[6-(2-dimethylamino-ethyl)-pyridine-3-sulfonyl]-piperidin-4-ylamino}-thiazol-5-yl)-(2,6-difluoro-phenyl)-methanone TFA Salt.

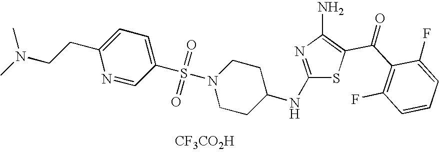

The title compound was prepared in a manner analogous to Example N1. {4-amino-2-[1-(6-vinyl-pyridine-3-sulfonyl)-piperidin-4-ylamino]-thiazol-5-yl}-(2,6-difluoro-phenyl)-methanone (Example I15; 100 mg, 0.198 mmol) and dimethylamine hydrochloride (65 mg, 0.79 mmol) gave 78 mg of white solid in 72% yield.

¹H NMR (DMSO-d₆): δ 9.45 (bs, 1H), 8.83 (s, 1H), 8.15 (d, 1H, J=8.3 Hz), 8.0 (bs, 2H), 7.64 (d, 1H, J=8.3 Hz), 7.48 (m, 1H), 7.14 (dd, 2H, J=7.7, 8.0 Hz), 3.30 (dd, 2H, J=7.2, 7.9 Hz),2.84 (d, 6H, J=4.8 Hz). ESIMS. (M−H⁺): 549. Anal. Calcd. for C₂₄H₂₈F₂N₆O₃S₂.1.9 TFA: C, 43.52; H, 3.93; N, 10.95; S, 8.36. Found: C, 43.35; H, 4.15; N, 10.92; S, 8.50.

### Example N12

### (4-Amino-2-{1-[2-(2-dimethylamino-ethyl)-pyrimidine-5-sulfonyl]-piperidine-4-ylamino}-thiazol-5-yl)-(2,6-difluoro-phenyl)-methanone Hydrochloride Salt.

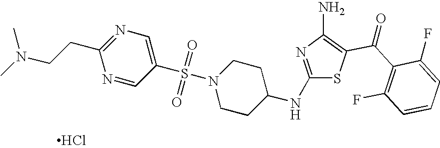

The title compound was prepared in a manner similar to that of Example N1 from {4-Amino-2-[1-(2-vinyl-pyrimidine-5-sulfonyl)-piperidin-4-ylamino]-thiazol-5-yl}-(2,6-difluoro-phenyl)-methanone (Example I16) and dimethylamine hydrochloride.

¹H NMR (CD₃OD): δ 9.14 (s, 1H), 7.66 (m, 1H), 7.16 (m, 2H), 3.76 (m, 4H), 3.60 (m, 2H), 8.01 (bs, 1H), 3.00 (s, 6H), 2.84 (m, 2H), 2.16 (m, 2H), 1.78 (m, 2H). LC-ESIMS (MH⁺): 552 Anal. Calcd. for C₂₃H₂₇F₂N₇O₃S₂.1.10 H₂O.4.0 HCl: C, 38.51; H, 4.67; N, 13.67; S, 8.94. Found: C, 38.64; H, 4.94; N, 13.34; S, 9.07.

Synthetic Protocol for Examples O through R Prepared in Parallel:

A stock solution of [4-amino-2-(piperidin in-4-ylamino)-thiazol-5-yl]-(2,6-difluoro-phenyl)-methanone (Example A6; 0.05 M, 200 μl)in acetonitrile was distributed into each well of 96 deep-well plates.

For the compounds of Examples O, in Table 2, stoichiomertric amounts of commercially available isocyanates were added and conditions similar to that for Example B1 were employed.

For the compounds of Examples P, in Table 3, stoichiometric amounts of commercially available sulfonyl chlorides were added and conditions similar to that for Example F1 were employed.

For the compounds of Examples Q, in Table 4, stoichiometric amounts of commercially available acyl chlorides were added and conditions similar to that for Example C1 were employed.

For the Examples R, in Table 5, stoichiometric amounts of both commercially available carboxylic acids, coupling reagents such as PyBOP or HATU were added, and conditions similar to that for Example D1 were employed.

The plates were gently shaken overnight at room temperature. The solvent was then removed with a GeneVac drying system to give the designated compounds, which were submitted for the bioassays without further purification.

Synthetic Protocol for Examples S:

The compounds of Examples S, in Table 6, were made in library format. Each well (1 ml) of J-Kem glass plates with [4-amino-2-(1-ethenesulfonyl-piperidin-4-ylamino)-thiazol-5-yl]-(2,6-difluoro-phenyl)-methanone (Example F55; 10 μmmol) and anhydrous DMSO (50 μL) were added a corresponding different amine (60 μmmol). Each plate was sealed with a Kem-Lab septum plate cover and heated at 100° C. for 15 hours in J-Kem reaction blocks. The plates were allowed to cool, dried in a Genevac HTS-12 high-speed evaporator, each well examined by LCMS, and submitted for bioassay without any further purification.

### Example T1

### 1-{4-[4-Amino-5-(2,6-difluoro-benzoyl)-thiazol-2-ylamino]-piperidin-1-yl}-propenone

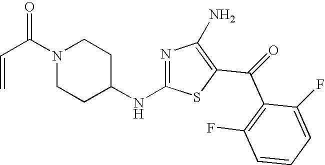

The title compound was prepared as follows. To a solution of [4-Amino-2-(piperidin-4-ylamino)-thiazol-5-yl]-(2,6-difluoro-phenyl)-methanone (Example ??; 8.0 g, 23.7 mmol) in THF (400 mL) were added triethylamine (6.60 mL, 47.3 mmol), the mixture was stirred at 0° C., acryloyl chloride (2.5 mL, 30.8 mmol) in THF (80 mL) was added dropwise. The mixture was stirred at 0° C. for half hour, then acidified with 1N HCl, the solvent was evaporated. The residue was partitioned between 10% MeOH/CH₂Cl₂ and 1N HCl, the organic layer was dried over Na₂SO₄, concentrated and purified by flash column with 0 to 5% MeOH/CH₂Cl₂ to give the title compound as a white powder in 57% yield, which was used without any further purification.

ESMS(M+H): 393.

### Example U1

### {2-[1-(3-Allylamino-propane-1-sulfonyl)-piperidin-4-ylamino]-4-amino-thiazol-5-yl}-(2,6-difluoro-phenyl)-methanone. Trifluoroacetic Acid Salt

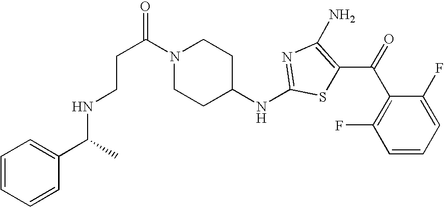

The title compound was prepared as follows. To a solution of 1-{4-[4-Amino-5-(2,6-difluoro-benzoyl)-thiazol-2-ylamino]-piperidin-1-yl}-propenone (Example T1; 314 mg, 0.800 mmol) in DMSO (4 mL) were added (S)-(−)-α-methylbenzylamine (310 μL, 2.40 mmol). The mixture heated at 100° C. for 24 hours, cooled, then the mixture was extracted with ethyl acetate, the organic layer was dried over Na₂SO₄, concentrated, the residue was diluted with water (50 mL) and stirred rapidly for one hour, the solid was filtered and washed with water, dried over vacuum to give the title compound as a white powder in 76% yield.

¹H NMR (DMSO-d₆): δ 8.72 (bs, 1H), 8.06 (bs, 2H), 7.51 (m, 1H), 7.21 (m, 1H), 7.18 (dd, 2H, J=7.8, 8.1 Hz), 4.21 (d, 1H, J=13.4 Hz), 3.76 (d, 1H, J=14.2 Hz), 3.69 (q, 1H, J=6.6 Hz), 3.06 (d, 1H, J=11.9 Hz), 1.21 (d, 3H, J=6.6 Hz). Anal. Calcd. for C₂₆H₂₉F₂N₅O₂S: C, 60.80; H, 5.69; N, 13.64; S, 6.24. Found: C, 60.99; H, 5.76; N, 13.43; S, 5.97. ESMS(M+H): 514.10.

### Example U2

### 1-{4-[4-Amino-5-(2,6-difluoro-benzoyl)-thiazol-2-ylamino]-piperidin-1-yl}-3-cyclohexylamino-propan-1-one. Acetic Acid Salt

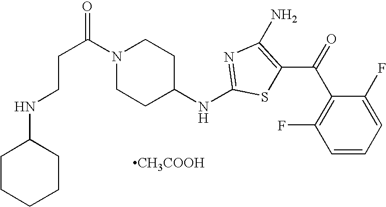

The title compound was prepared in a manner analogous to Example U1. The reaction of 1-{4-[4-Amino-5-(2,6-difluoro-benzoyl)-thiazol-2-ylamino]-piperidin-1-yl}-propenone (Example T1; 314 mg, 0.800 mmol) and cyclohexylamine (238 mg, 2.40 mmol) give the title compound as a white powder in 51% yield after preparative HPLC purification.

¹H NMR (DMSO-d₆): δ 8.71 (bs, 1H), 8.07 (bs, 2H), 7.50 (m, 1H), 7.17 (dd, 2H, J=7.7, 7.9 Hz), 4.22 (d, 1H, J=11.7 Hz), 3.82 (d, 1H, J=13.2 Hz), 2.73 (dd, 3H, J=6.4, 7.0 Hz), 1.77 (d, 2H, J=10.4 Hz). Anal. Calcd. for C₂₄H₃₁F₂N₅O₂S.1.3 CH₃COOH: C, 54.87; H, 6.29; N, 11.94; S, 5.47. Found: C, 54.69; H, 6.52; N, 12.17; S, 5.51. ESMS(M+H): 492.20.

### Example U3

### 1-{4-[4-Amino-5-(2,6-difluoro-benzoyl)-thiazol-2-ylamino]-piperidin-1-yl}-3-(methyl-pyridin-3-ylmethyl-amino)-propan-1-one. Acetic Acid Salt

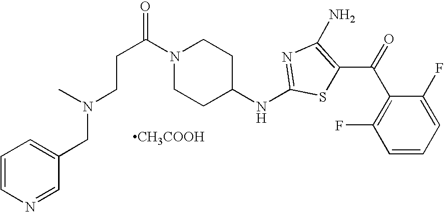

The title compound was prepared in a manner analogous to Example U1. The reaction of 1-{4-[4-Amino-5-(2,6-difluoro-benzoyl)-thiazol-2-ylamino]-piperidin-1-yl}-propenone (Example T1; 314 mg, 0.800 mmol) and 3-picolylmethylamine (293 mg, 2.40 mmol) give the title compound as a light yellow powder in 53% yield after preparative HPLC purification.

¹H NMR (DMSO-d₆): δ 8.76 (bs, 1H), 8.47 (d, 1H, J=1.5 Hz), 8.46 (dd, 1H, J=1.5, 4.7 Hz), 8.08 (bs, 2H), 7.68 (d, 1H, J=7.7 Hz), 7.50 (m, 1H), 7.35 (dd, 1H, J=4.7, 7.7 Hz), 7.17 (dd, 2H, J=7.7, 8.1 Hz), 4.21 (d, 1H, J=13.6 Hz), 3.82 (d, 1H, J=14.0 Hz), 3.51 (s, 2H), 3.08 (dd, 1H, J=11.8, 12.8 Hz), 2.72 (dd, 1H, J=8.5, 12.8 Hz), 2.13 (s, 3H). Anal. Calcd. for C₂₅H₂₈F₂N₆O₂S.0.5 CH₃COOH.1.0 H₂O: C, 55.50; H, 5.73; N, 14.94; S, 5.70. Found: C, 55.76; H, 5.64; N, 15.16; S, 5.68. ESMS(M+H): 515.15.

### Example U4

### (4-Amino-2-{1-[3-(cyclohexyl-methyl-amino)-propane-1-sulfonyl]-piperidin-4-ylamino}-thiazol-5-yl)-(2,6-difluoro-phenyl)-methanone. Acetic Acid Salt

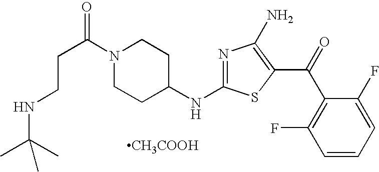

The title compound was prepared as follows. 1-{4-[4-Amino-5-(2,6-difluoro-benzoyl)-thiazol-2-ylamino]-piperidin-1-yl}-propenone (Example T1; 250 mg, 0.64 mmol) and t-butylamine (0.4 mL) was heated in microwave at 120° C. for half hour, HPLC showed only one third conversion of the starting material, another 0.4 mL of t-butylamine was added, the mixture was heated at 120° C. (30 min.×3 times) until HPLC showed the reaction was completed. The solvent was evaporated, the residue was purified by preparative HPLC to give the title compound as a white powder in 70% yield.

¹H NMR (DMSO-d₆): δ7.99 (bs, 2H), 7.41 (m, 1H), 7.08 (dd, 2H, J=7.5, 8.1 Hz), 4.15 (d, 1H, J=13.8 Hz), 3.71 (d, 2H, J=14.3 Hz), 3.01 (dd, 2H, J=11.5, 12.1 Hz), 2.64 (dd, 1H, J=12.1, 15.1 Hz), 2.58 (dd, 2H, J=6.6, 7.0 Hz), 0.94 (s, 9H). Anal. Calcd. for C₂₂H₂₉F₂N₅O₂S.1.0 CH₃COOH.0.2 CH₂Cl₂: C, 53.57; H, 6.20; N, 12.91; S, 5.91. Found: C, 53.40; H, 6.32; N, 13.07; S, 5.94. ESMS(M+H): 466.10.

Synthetic Protocol For Examples V:

The compounds of Examples V, in Table 7, were made in library format. Stock solutions were respectively prepared; 1.2 M of assorted amines separately in anhydrous DMSO and 0.4 M of iodide Example F45 in anhydrous DMSO. For each reaction vessel in a library array was added in succession, a solution of iodide Example F45 (200 μL, 0.08 mmol, 1 equiv.), each respective amine solution (200 μL, 0.24 mmol, 3 equiv.), and a magnetic stir bar. The vessels were covered with cellophane and stirred at 100° C. overnight (16 hours). The vessels were allowed to cool and then the solvents and volatiles removed in vacuo with moderate heating (30-40° C.). DMSO containing 0.01% 2,6-di-tert-butyl-4-methylphenol (BHT; 0.6 mL) was added to each vessel, covered with cellophane, completely dissolved using a Vortex shaker, prior to removing 10 μL aliquots—that were each diluted to 1.0 mL with 95:5 MeOH/H₂O, agitated to homogenize each, and submitted for preparative HPLC purification.

Selected examples were purified via preparative HPLC, and their respective NMR recorded as given below:

### Example V177

### (4-Amino-2-{[1-({3-[methyl(2-methyl-2-propen-1-yl)amino]propyl}sulfonyl)-4-piperidinyl]amino}-1,3-thiazol-5-yl)(2,6-difluorophenyl)methanone.

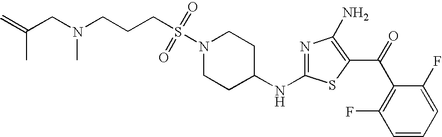

¹H NMR (300 MHz, DMSO-d₆) δ ppm 8.71 (s, 1 H) 8.00 (s, 2 H) 7.43 (ddd, J=15.20, 8.40, 6.80 Hz, 1 H) 7.10 (t, J=7.74 Hz, 2 H) 4.88 (s, 2 H) 3.42-3.53 (m, 3 H) 3.00 (s, 3 H) 2.87 (t, J=11.14 Hz, 2 H) 1.76-1.98 (m, 4 H) 1.65 (s, 3 H) 1.41 (q, 2 H).

### Example V178

### (4-Amino-2-{[1-({3-[(3-fluorobenzyl)amino]propyl}sulfonyl)-4-piperidinyl]amino}-1,3-thiazol-5-yl)(2,6-difluorophenyl)methanone.

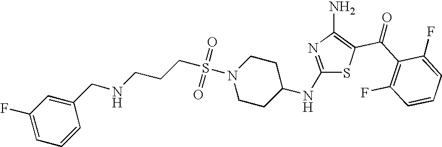

¹H NMR (300 MHz, DMSO-d₆) δ ppm 8.78 (s, 1 H) 8.06 (s, 2 H) 7.37-7.55 (m, 2 H) 7.10-7.32 (m, 5 H) 3.99 (s, 2 H) 3.08-3.19 (m, J=8.12, 6.80 Hz, 2 H) 2.87 (s, 4 H) 1.82-2.02 (m, 4 H) 1.44 (q, J=10.01 Hz, 2 H).

### Example V179

### (4-Amino-2-{[1-({3-[(2-furylmethyl)(methyl)amino]propyl}sulfonyl)-4-piperidinyl]amino}-1,3-thiazol-5-yl)(2,6-difluorophenyl)methanone.

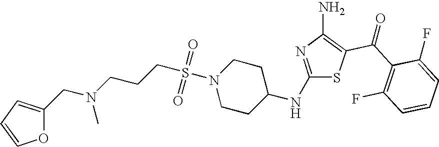

¹H NMR (300 MHz, DMSO-d₆) δ ppm 8.77 (s, 1 H) 8.07 (s, 2 H) 7.62 (s, 1H) 7.48 (ddd, J=15.20, 8.31, 6.70 Hz, 1H) 7.15 (t, J=7.84 Hz, 2 H) 6.42 (t,1 H) 6.34 (s, 1 H) 3.67 (s, 2 H) 3.51 (d, J=12.65 Hz, 2 H) 3.02 (t, 2 H) 2.92 (t, J=10.76 Hz, 2 H) 2.23 (s, 3 H) 1.94 (d, J=8.69 Hz, 2 H) 1.84 (t, J=6.99 Hz, 2 H) 1.46 (q, J=10.14 Hz, 2 H).

### Example V180

### (4-Amino-2-{[1-({3-[(3-methylbenzyl)amino]propyl}sulfonyl)-4-piperidinyl]amino}-1,3-thiazol-5-yl)(2,6-difluorophenyl)methanone.

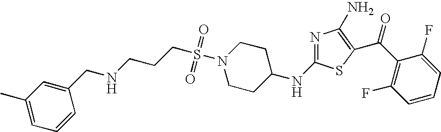

¹H NMR (300 MHz, DMSO-d₆) δ ppm 8.77 (s, 1 H) 8.06 (s, 2 H) 7.49 (ddd, J=15.20, 8.40, 6.80 Hz, 1 H) 7.24-7.32 (m, 1 H) 7.11-7.23 (m, 4 H) 3.93 (s, 2 H) 3.12 (t, J=7.93, 6.99 Hz, 2 H) 2.80-2.97 (m, 4 H) 2.30 (s, 3 H) 1.84-2.02 (m, 4 H) 1.48 (q, J=10.51 Hz, 2 H).

### Example V181

### (4-Amino-2-{[1-({3-[benzyl(methyl)amino]propyl}sulfonyl)-4-piperidinyl]amino}-1,3-thiazol-5-yl)(2,6-difluorophenyl)methanone.

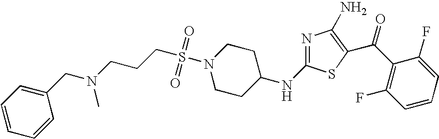

¹H NMR (300 MHz, DMSO-d₆) δ ppm 8.89 (s, 1 H) 8.07 (s, 2 H) 7.39-7.59 (m, 6 H) 7.15 (t, J=7.74 Hz, 2 H) 4.20 (s, 2 H) 3.76-3.99 (m, 1 H) 3.52 (d, J=10.58 Hz, 2 H) 3.12 (s, 4 H) 2.93 (t, J=11.14 Hz, 2 H) 2.61 (s, 2 H) 2.06 (s, 2 H) 1.95 (d, J=7.37 Hz, 2 H) 1.47 (q, J=9.82 Hz, 2 H).

### Example V182

### (4-Amino-2-{[1-({3-[(4-fluorobenzyl)amino]propyl}sulfonyl)-4-piperidinyl]amino}-1,3-thiazol-5-yl)(2,6-difluorophenyl)methanone.

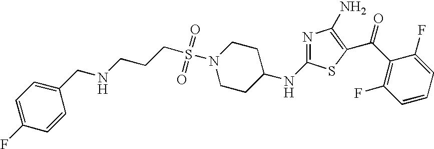

¹H NMR (300 MHz, DMSO-d₆) δ ppm 8.76 (s, 1 H) 7.96-8.16 (m, J=0.94 Hz, 2 H) 7.48 (ddd, J=15.16, 8.36, 6.70 Hz, 1 H) 7.34 (dd, J=8.50, 5.85 Hz, 2 H) 7.05-7.22 (m, 4 H) 3.64 (s, 2 H) 3.46-3.55 (m, J=12.46 Hz, 1 H) 3.01-3.11 (m, 2 H) 2.90 (t, J=10.86 Hz, 2 H) 1.88-2.01 (m, J=7.18 Hz, 2 H) 1.69-1.84 (m, 2 H) 1.46 (q, J=9.82 Hz, 2 H).

### Example V183

### (4-Amino-2-{[1-({3-[(2-fluorobenzyl)amino]propyl}sulfonyl)-4-piperidinyl]amino}-1,3-thiazol-5-yl)(2,6-difluorophenyl)methanone.

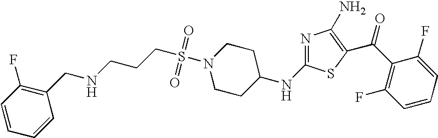

¹H NMR (300 MHz, DMSO-d₆) δ ppm 8.86 (s, 2 H) 8.06 (s, 2 H) 7.62 (td, J=7.65,1.13 Hz, 1 H) 7.44-7.53 (m, 2 H) 7.40 (dd, J=10.29, 8.97 Hz, 1 H) 7.30 (t, 1 H) 7.16 (t, J=7.84 Hz, 2 H) 4.18 (s, 2 H) 3.53 (d, J=12.09 Hz, 2 H) 3.13-3.22 (m, J=7.18, 6.42 Hz, 2 H) 3.05 (t, J=7.37 Hz, 2 H) 2.92 (t, J=10.86 Hz, 2 H) 2.06 (t, J=10.20, 7.18 Hz, 2 H) 1.98 (d, J=15.49 Hz, 1 H) 1.48 (q, J=10.32 Hz, 2 H).

### Example V184

### (4-Amino-2-{[1-({3-[(2-methylbenzyl)amino]propyl}sulfonyl)-4-piperidinyl]amino}-1,3-thiazol-5-yl)(2,6-difluorophenyl)methanone.

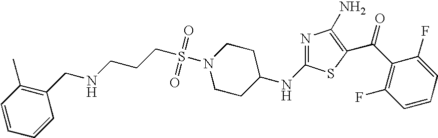

¹H NMR (300 MHz, DMSO-d₆) δ ppm 8.75 (s, 1 H) 8.00 (s, 1 H) 7.43 (t, J=7.74 Hz, 1 H) 7.30 (dd, J=8.59, 1.98 Hz, 1 H) 7.05-7.17 (m, 5 H) 3.82 (s, 1 H) 3.47 (d, J=12.28 Hz, 1 H) 3.07 (t, J=7.37 Hz, 1 H) 2.83 (s, 4 H) 2.26 (s, 3 H) 1.81-1.95 (m, 3 H) 1.42 (q, J=9.82 Hz, 2 H).

### Example V185

### (4-Amino-2-{[1-({3-[(1-phenylethyl)amino]propyl}sulfonyl)-4-piperidinyl]amino}-1,3-thiazol-5-yl)(2,6-difluorophenyl)methanone.

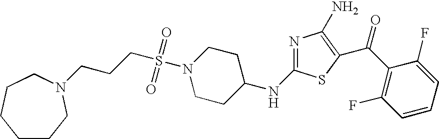

¹H NMR (300 MHz, DMSO-d₆) δ ppm 8.77 (s, 1 H) 8.06 (s, 2 H) 7.49 (ddd, J=15.25, 8.36, 6.80 Hz, 1 H) 7.25-7.44 (m, 5 H) 7.15 (t, 2 H) 3.99 (s, 1 H) 3.06 (d, J=5.48 Hz, 2 H) 2.86 (t, J=10.86 Hz, 2 H) 2.62 (s, 1 H) 1.92 (s, 2 H) 1.76-1.87 (m, 2 H) 1.39-1.52 (m, 2 H) 1.35 (d, J=6.42 Hz, 3 H).

Biochemical and Biological Evaluation:

Cyclin-dependent kinase activity was measured by quantifying the enzyme-catalyzed, time-dependent incorporation of radioactive phosphate from [³²P]ATP or [³³P]ATP into a protein substrate. Unless noted otherwise, assays were performed in 96-well plates in a total volume of 50 μL, in the presence of 10 mM HEPES (N-[2-hydroxyethyl]piperazine-N′-[2-ethanesulfonic acid]) (pH 7.4), 10 mM MgCl₂, 25 μM adenosine triphosphate (ATP), 1 mg/mL ovalbumin, 5 μg/mL leupeptin, 1 mM dithiothreitol, 10 mM β-glycerophosphate, 0.1 mM sodium vanadate, 1 mM sodium fluoride, 2.5 mM ethylene glycol-bis(β-aminoethyl ether)-N,N,N′N′-tetraacetic acid (EGTA), 2% (v/v) dimethylsulfoxide, and 0.03-0.4 μCi [^{32/33}P]ATP per reaction. Reactions were initiated with enzyme, incubated at 30° C., and terminated after 20 minutes by the addition of ethylenediaminetetraacetic acid (EDTA) to 250 mM. The phosphorylated substrate was then captured on a nitrocellulose or phosphocellulose membrane using a 96-well filtration manifold, and unincorporated radioactivity was removed by repeated washing with 0.85% phosphoric acid. Radioactivity was quantified by exposing the dried membranes to a phosphorimager.

Compounds from combinatorial libraries were screened from 96-well plates for % inhibition of CDK activity at 30 nM theoretical compound concentration. Inhibition was measured relative to control wells that contained all reaction components including 2% (v/v) DMSO but no compound, after subtraction of background radioactivity measured in the absence of enzyme. Apparent Kᵢ values of discrete compounds were measured by assaying enzyme activity in the presence of different inhibitor compound concentrations and subtracting the background radioactivity measured in the absence of enzyme. The kinetic parameters (kcat, Kₘ for ATP) were measured for each enzyme under the usual assay conditions by determining the dependence of initial rates on ATP concentration. Inhibition data were fit to an equation for competitive inhibition using Kaleidagraph (Synergy Software), or were fit to an equation for competitive tight-binding inhibition using the software KineTic (BioKin, Ltd.).

Inhibition of CDK4/Cyclin D Retinoblastoma Kinase Activity:

A complex of human CDK4 and genetically truncated (1-264) cyclin D3 was purified using traditional biochemical chromatographic techniques from insect cells that had been co-infected with the corresponding baculovirus expression vectors (see e.g., Meijer and Kim, “Chemical Inhibitors of Cyclin-Dependent Kinases,” Methods in Enzymol,. vol. 283 (1997), pp. 113-128.). The enzyme complex (5 nM) was assayed with 0.3-0.5 μg of purified recombinant retinoblastoma protein fragment (Rb) as a substrate. The engineered Rb fragment (residues 386-928 of the native retinoblastoma protein; 62.3 kDa) contains the majority of the phosphorylation sites found in the native 106-kDa protein, as well as a tag of six histidine residues for ease of purification. Phosphorylated Rb substrate was captured by microfiltration on a nitrocellulose membrane and quantified using a phosphorimager as described above. For measurement of tight-binding inhibitors, the assay duration was extended to 60 minutes, during which the time-dependence of product formation was linear and initial rate conditions were met. Kᵢ values for the compounds of Example A through Example N were measured as described above and shown in Table 1. Percent inhibitions for the compounds of Example O through R were calculated as described above and shown in Table 2.

Inhibition of CDK2/Cyclin A Retinoblastoma Kinase Activity:

CDK2 was purified using published methodology (Rosenblatt et al., “Purification and Crystallization of Human Cyclin-dependent Kinase 2,” J. Mol. Biol., vol. 230, 1993, pp. 1317-1319) from insect cells that had been infected with a baculovirus expression vector. Cyclin A was purified from E. coli cells expressing full-length recombinant cyclin A, and a truncated cyclin A construct was generated by limited proteolysis and purified as described previously (Jeffrey et al., “Mechanism of CDK activation revealed by the structure of a cyclin A-CDK2 complex,” Nature, vol. 376 (27 Jul. 1995), pp. 313-320). A complex of CDK2 and proteolyzed cyclin A was prepared and purified by gel filtration. The substrate for this assay was the same Rb substrate fragment used for the CDK4 assays, and the methodology of the CDK2/delta cyclin A and the CDK4/delta cyclin D3 assays was essentially the same, except that CDK2 was present at 10 nM or 19 nM. The duration of the assay was 60 or 75 minutes, during which the time-dependence of product formation was linear and initial rate conditions were met. Kᵢ values of the compounds of Example A through Example N were measured as described above and shown in Table 1. And, the percent inhibitions of the compounds of Example O through Example R were calculated as described above and shown in Table 2.

Inhibition of CDK1(cdc2)/Cyclin B Histone H1 Kinase Activity:

The complex of human CDK1 (cdc2) and cyclin B was purchased from New England Biolabs (Beverly Mass.). Alternatively, a CDK1/glutathione-S-transferase-cyclin B1 complex was purified using glutathione affinity chromatography from insect cells that had been co-infected with the corresponding baculovirus expression vectors. The assay was executed as described above at 30° C. using 2.5 units of cdc2/cyclin B, 10 μg Histone Hi protein, and 0.1-0.3 μCi [^{32/33}P]ATP per assay. Phosphorylated histone substrate was captured by microfiltration on a phosphocellulose P81 membrane and quantified using a phosphorimager as described above. Kᵢ values were measured using the described curve-fitting programs. The results are shown in Table 6.

Inhibition of Cell Growth: Assessment of Cytotoxicity:

Inhibition of cell growth was measured using the tetrazolium salt assay, which is based on the ability of viable cells to reduce 3-(4,5-dimethylthiazol-2-yl)-2,5-[2H]-diphenyltetrazolium bromide (MTT) to formazan (Mossman, Journal of Immunological Methods, vol. 65 (1983), pp. 55-58). The water-insoluble purple formazan product was then detected spectrophotometrically. The HCT-116 cell line was used as a representative cancer cell line and grown in 96-well plates. Cells were plated in McCoy's 5A Medium at a volume of 135 μl/well. Plates were incubated for four hours before addition of inhibitor compounds. Different concentrations of inhibitor compounds were added in 0.5% (v/v) dimethylsulfoxide (15 μLuwell), and cells were incubated at 37° C. (5% CO₂) for three to five days. At the end of the incubation, MTT was added to a final concentration of 0.2 mg/mL, and cells were incubated for 4 hours more at 37° C. After centrifugation of the plates and removal of medium, the absorbance of the formazan (solubilized in dimethylsulfoxide) was measured at 540 nm. The concentration of inhibitor compound causing 50%(IC₅₀) or 90%(IC₅₀) inhibition of growth was determined from the linear portion of a semi-log plot of inhibitor concentration versus percent inhibition. All results were compared to control cells treated only with 0.5% (v/v) dimethylsulfoxide. The IC₅₀ and IC₉₀ of the compounds of Examples A through Example N are shown in Table 1. Percent inhibitions at 0.25 μM of the compounds of Example O were calculated and shown in Table 2. Percent inhibitions at 0.25 μM or 0.1 μM of the compounds of Example P through R were calculated and shown in Table 3 to Table 5.

For the compounds shown in Table 1 through Table 6, the group of —N(H)— and methyl (—CH₃) of the formulae are sometimes shown as “—N—” and “—” for simplicity, respectively, and the compounds in the form of salts are shown in their free base forms. In Tables 2 through Table 5, the straight line, for the purpose of these tables, designates the point of connection to the structure appearing at the tope of each Table. The straight line does not designate a methyl group. For example, in Table 2, the moiety indicated for R1 taken together with formula (1) appearing as Example O1 in Table 2 provides the following structure:

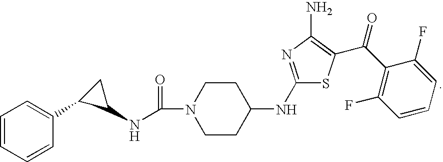

TABLE 1

| Exam- | | CDK2 | CDK4 | HCT-116 | HCT-116 |
|---|---|---|---|---|---|
| ple | STRUCTURE | Ki (μM) | Ki (μM) | IC50(μM) | IC50(μM) |
| A1 | 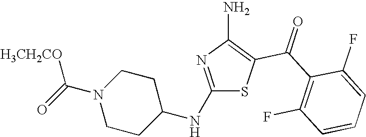 | 0.19 | 0.082 | NT | NT |
| A2 | 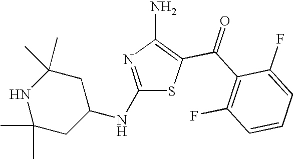 | >5 | >2 | NT | NT |
| A3 | 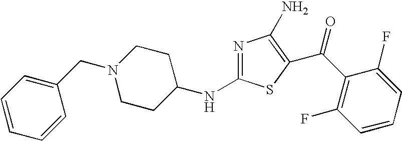 | 0.49 | 0.13 | 1.7 | 3.1 |
| A4 | 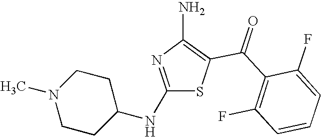 | 12 | 0.93 | 1.7 | 3.8 |
| A5 | 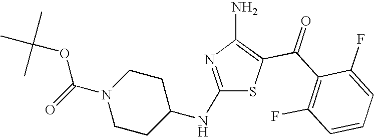 | NT | NT | NT | NT |
| A6 | 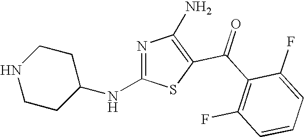 | 1 | 0.83 | NT | NT |
| A7 | 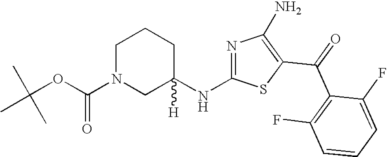 | NT | NT | NT | NT |
| A8 | 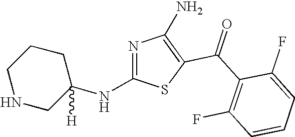 | NT | NT | NT | NT |
| A9 | 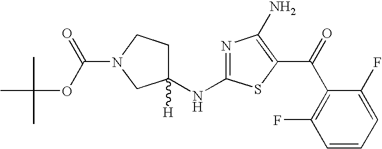 | NT | NT | NT | NT |
| A10 | 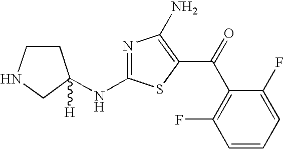 | NT | NT | NT | NT |
| A11 | 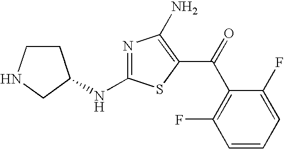 | NT | NT | NT | NT |
| A12 | 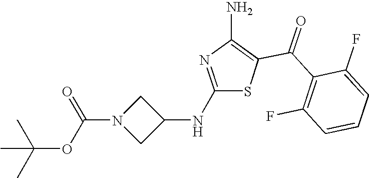 | NT | NT | NT | NT |
| A13 | 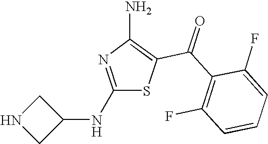 | NT | NT | NT | NT |
| A14 | 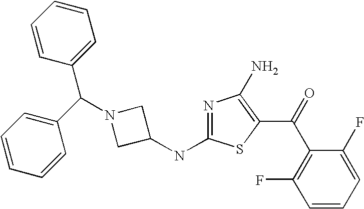 | >2 | >2 | >5 | >5 |
| B1 | 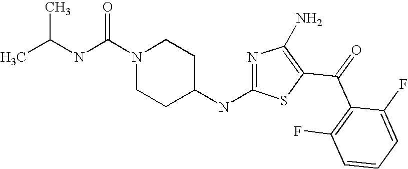 | 0.41 | 0.38 | NT | NT |
| B2 | 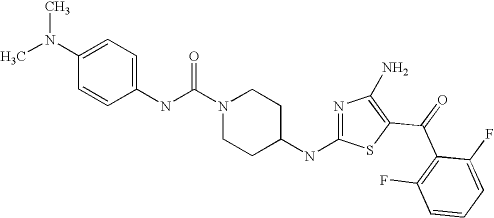 | 0.028 | 0.11 | 0.35 | 0.95 |
| B3 | 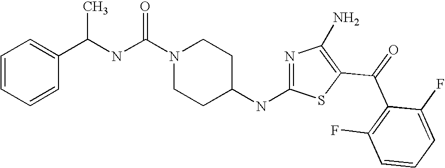 | 0.19 | 0.42 | NT | NT |
| B4 | 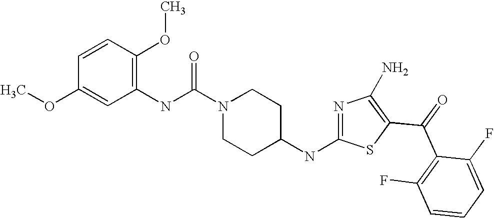 | 0.066 | 0.062 | NT | NT |
| C1 | 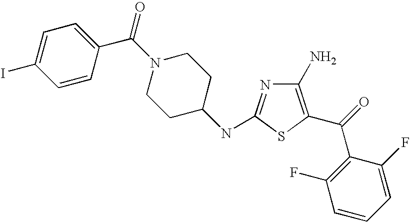 | 0.068 | 0.011 | 1.2 | 2.3 |
| C2 | 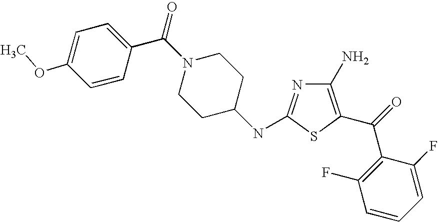 | 0.065 | 0.0096 | 0.77 | 1.9 |
| C3 | 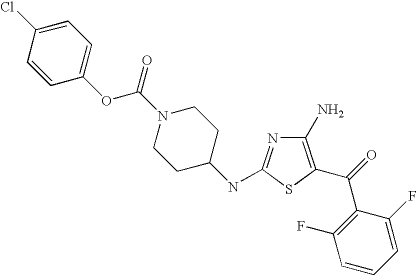 | 0.017 | 0.0037 | 0.33 | 1.2 |
| C4 | 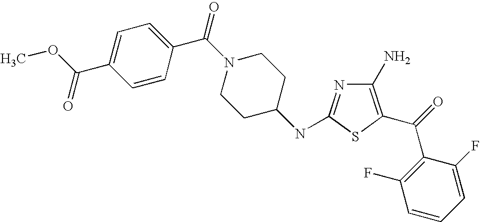 | 0.081 | 0.011 | 0.8 | 2 |
| C5 | 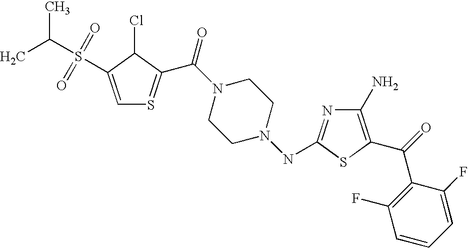 | 0.081 | 0.008 | 1.9 | 4 |
| C6 | 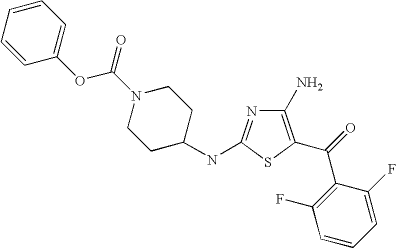 | 0.0061 | 0.0079 | 0.22 | 0.9 |
| C7 | 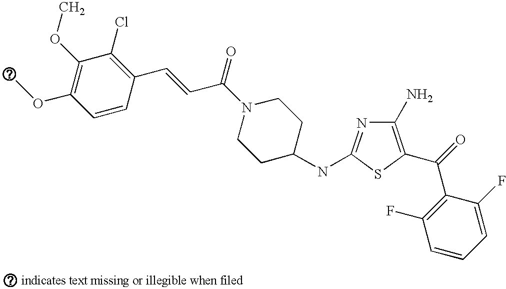 | 0.032 | 0.04 | 0.6 | 1.6 |
| C8 | 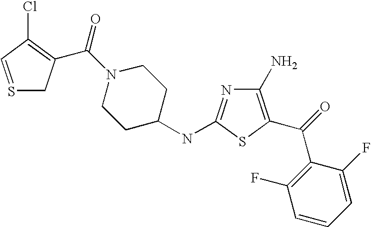 | 0.045 | 0.041 | 0.46 | 1.3 |
| C9 | 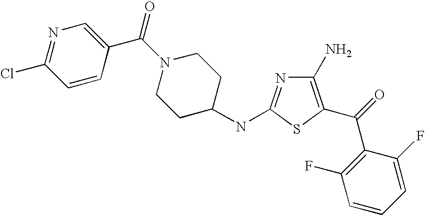 | 0.067 | 0.02 | 0.59 | 1.3 |
| C10 | 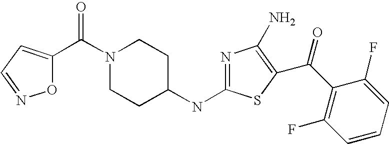 | 0.039 | 0.022 | 0.75 | 2.1 |
| C11 | 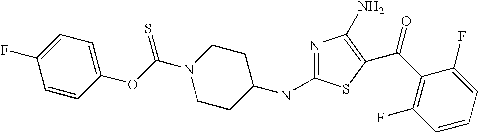 | 0.0065 | 0.01 | 0.4 | 2.7 |
| C12 | 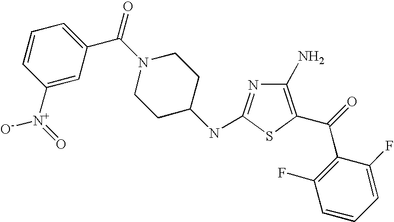 | 0.059 | 0.012 | 0.22 | 0.51 |
| C13 | 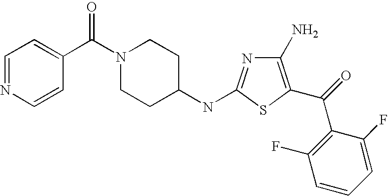 | 0.053 | 0.018 | 2.8 | 5 |
| C14 | 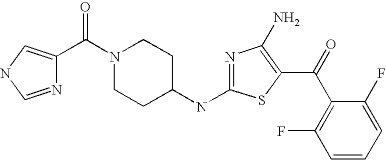 | 0.095 | 0.066 | >5 | >5 |
| C15 | 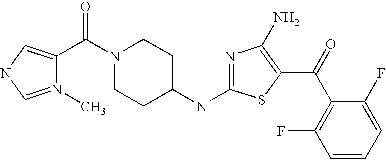 | 0.15 | 0.051 | >5 | >5 |
| C16 | 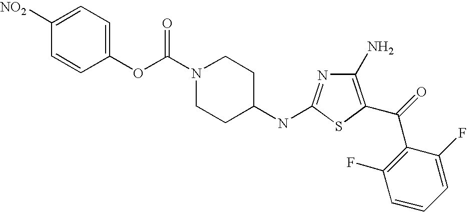 | 0.018 | 0.0075 | 0.13 | 0.4 |
| C17 | 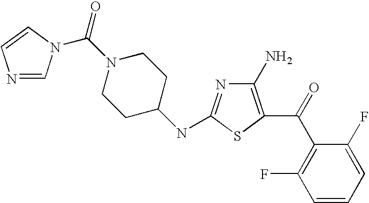 | 0.017 | 0.021 | 2.1 | 4.4 |
| C18 | 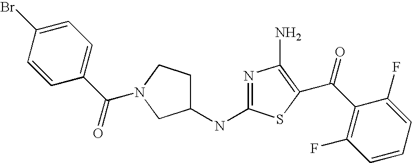 | 0.077 | 0.21 | NT | NT |
| C19 | 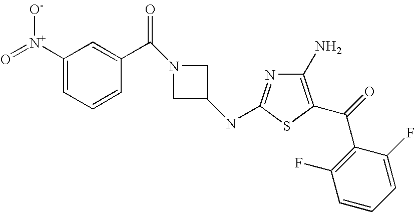 | 0.36 | 0.66 | 3.2 | 4.8 |
| D1 | 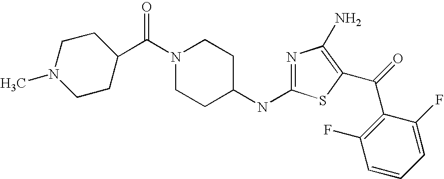 | 0.46 | 0.13 | >5 | NT |
| D2 | 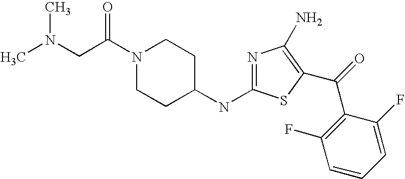 | 1.3 | 0.12 | 1.9 | 5 |
| D3 | 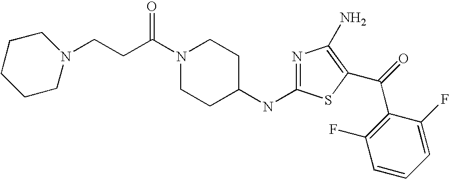 | 0.4 | 0.071 | >5 | NT |
| D4 | 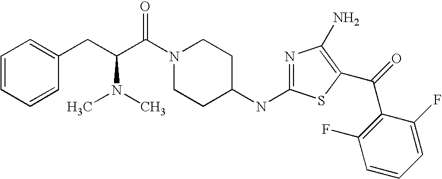 | 2.6 | 0.46 | >5 | NT |
| D5 | 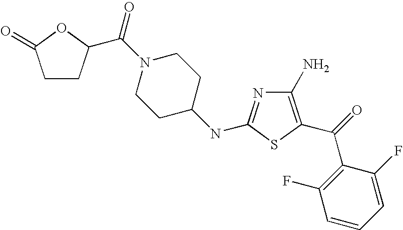 | 0.0064 | 0.0068 | >5 | >5 |
| D6 | 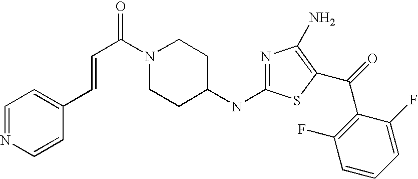 | 0.16 | 0.067 | 1.9 | 3.9 |
| D7 | 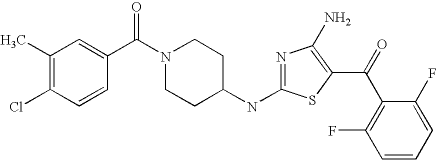 | 0.1 | 0.032 | 0.0072 | 0.22 |
| D8 | 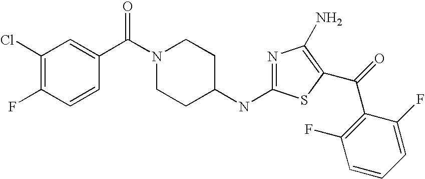 | 0.099 | 0.0096 | 0.097 | 0.25 |
| D9 | 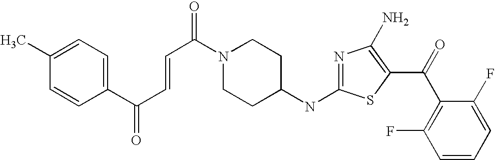 | 0.51 | 0.15 | NT | NT |
| D10 | 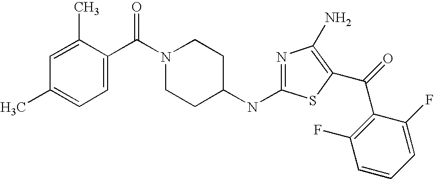 | 0.085 | 0.062 | 0.06 | 0.2 |
| D11 | 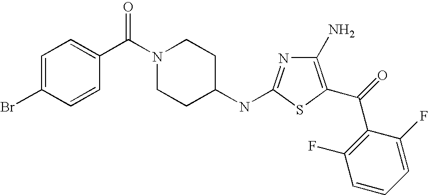 | 0.081 | 0.031 | 0.72 | 1.8 |
| D12 | 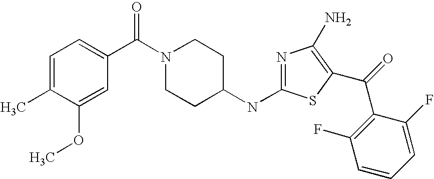 | 0.029 | 0.014 | 0.12 | 0.32 |
| D13 | 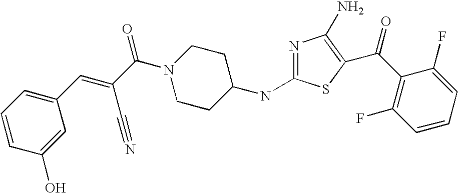 | 0.024 | 0.0018 | 1.3 | 5 |
| D14 | 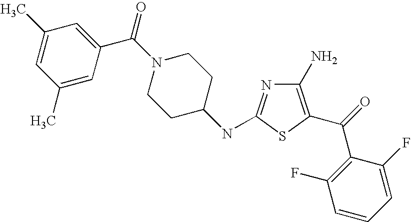 | 0.12 | 0.019 | 0.014 | 0.041 |
| D15 | 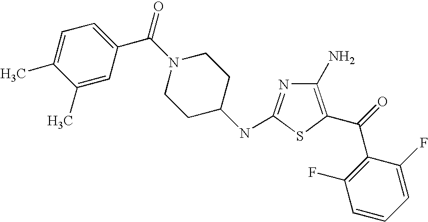 | 0.17 | 0.027 | 0.05 | 0.17 |
| D16 | 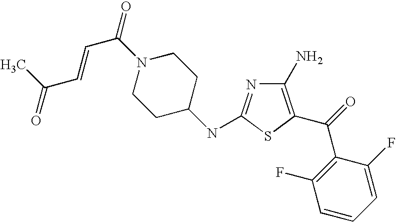 | 0.5 | 0.14 | 0.082 | 0.15 |
| D17 | 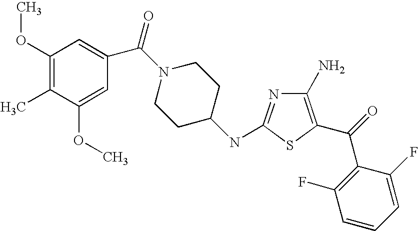 | 0.069 | 0.018 | 0.057 | 0.16 |
| D18 | 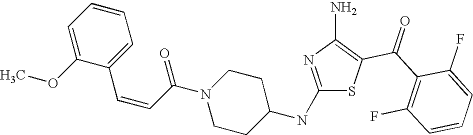 | 0.054 | 0.018 | NT | NT |
| D19 | 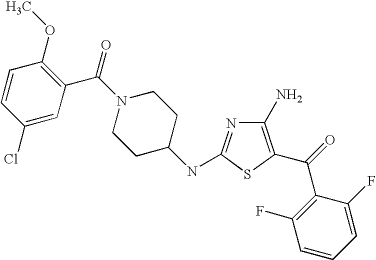 | 0.105 | 0.079 | NT | NT |
| E1 | 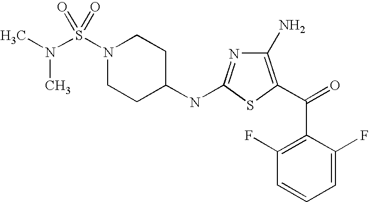 | 0.014 | 0.022 | NT | NT |
| E2 | 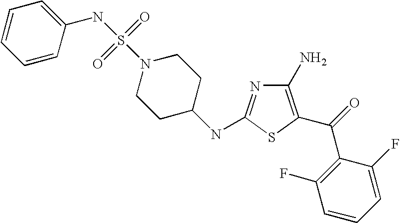 | 0.0012 | 0.0039 | 0.68 | 1.3 |
| E3 | 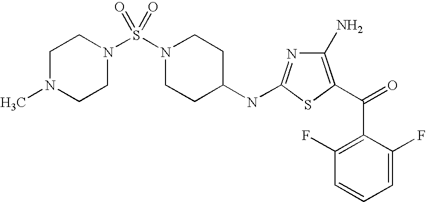 | 0.012 | 0.0054 | 0.33 | 0.78 |
| E4 | 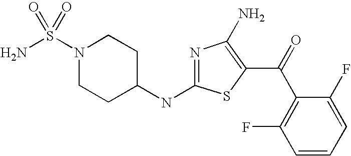 | 0.0027 | 0.014 | 0.57 | 1.2 |
| E5 | 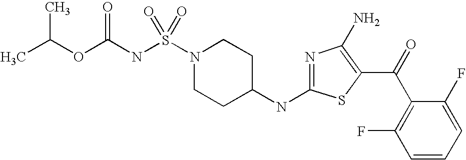 | 0.038 | 0.17 | >5 | >5 |
| F1 | 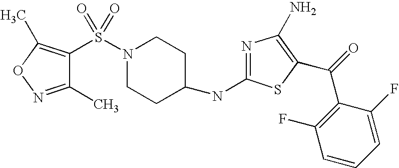 | 0.012 | 0.014 | 1.4 | 4.5 |
| F2 | 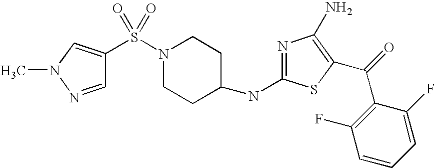 | <0.005 | 0.0019 | 1.3 | 4 |
| F3 | 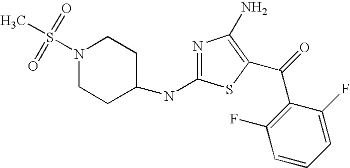 | 0.0029 | 0.0059 | 0.18 | 0.48 |
| F4 | 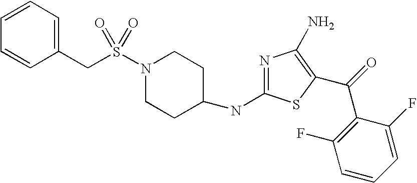 | 0.0041 | 0.0028 | 0.26 | 0.59 |
| F5 | 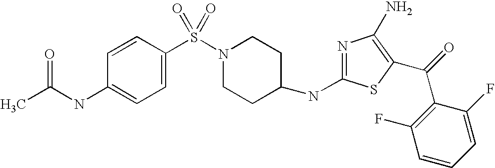 | <0.001 | 0.001 | 0.5 | 1.3 |
| F6 | 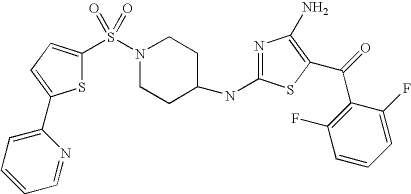 | 0.00043 | 0.00046 | 0.17 | 0.45 |
| F7 | 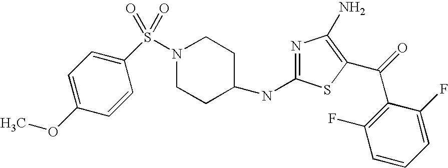 | 0.0008 | 0.0025 | 0.19 | 0.46 |
| F8 | 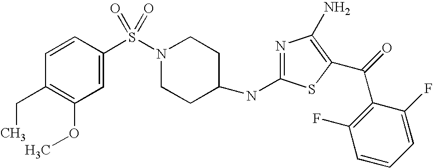 | <0.001 | 0.003 | 0.16 | 0.29 |
| F9 | 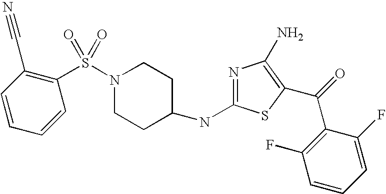 | 0.002 | 0.0036 | 0.14 | 0.25 |
| F10 | 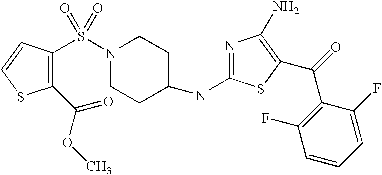 | 0.0079 | 0.0056 | 0.28 | <5 |
| F11 | 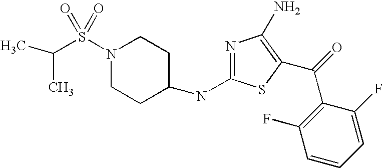 | 0.0016 | 0.0011 | 0.18 | 0.45 |
| F12 | 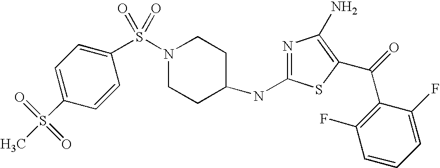 | 0.00037 | 0.0013 | 0.19 | 0.5 |
| F13 | 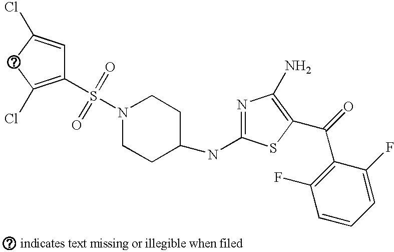 | 0.0087 | 0.0058 | 0.61 | 2.6 |
| F14 | 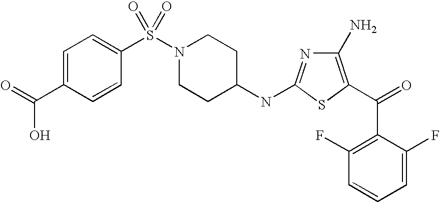 | 0.002 | 0.014 | <5 | <5 |
| F15 | 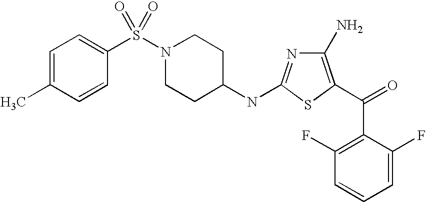 | 0.0028 | 0.0034 | 0.41 | 1.2 |
| F16 | 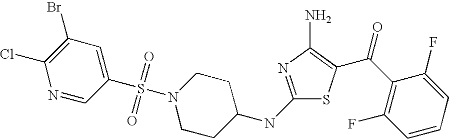 | NT | NT | NT | NT |
| F17 | 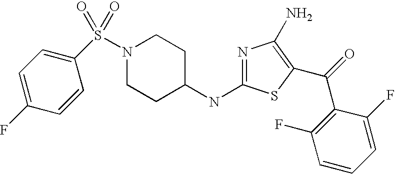 | <0.001 | 0.0014 | 0.07 | 0.23 |
| F18 | 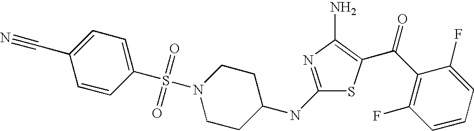 | <0.001 | 0.00098 | 0.3 | 0.5 |
| F19 | 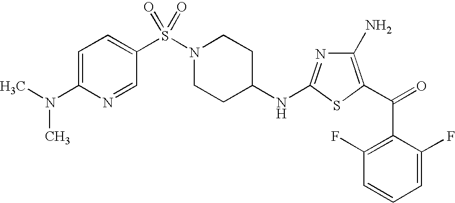 | 0.0032 | 0.0017 | 0.048 | 0.2 |
| F20 | 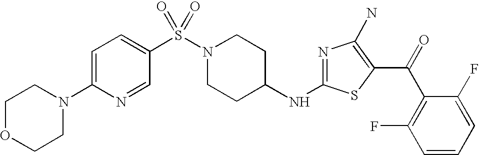 | 0.0014 | 0.0013 | 0.17 | 1.3 |
| F21 | 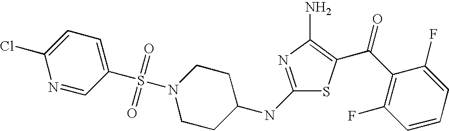 | 0.0017 | 0.0025 | NT | NT |
| F22 | 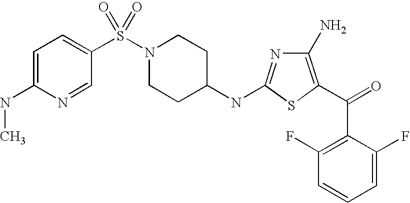 | 0.00084 | 0.0012 | 0.08 | 0.23 |
| F23 | 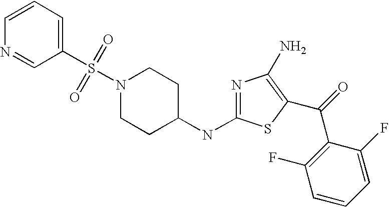 | 0.0028 | 0.0048 | 0.13 | 0.3 |
| F24 | 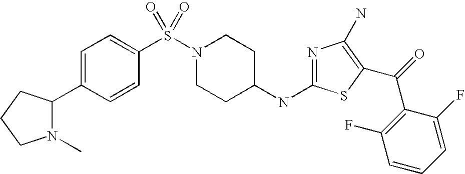 | <0.001 | 0.00034 | 0.59 | 1.6 |
| F25 | 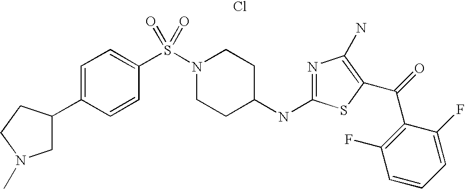 | 0.0015 | 0.00093 | 0.08 | 0.3 |
| F26 | 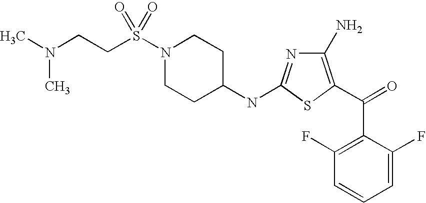 | 0.015 | 0.0022 | 0.28 | 0.65 |
| F27 | 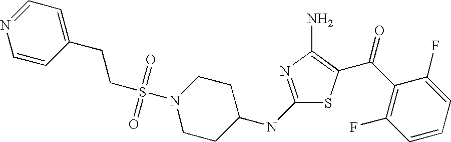 | 0.032 | 0.0068 | 1 | 5 |
| F28 | 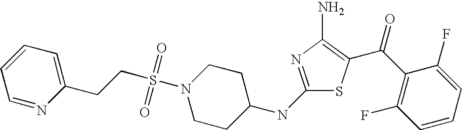 | 0.0036 | 0.0081 | 0.65 | 1.3 |
| F29 | 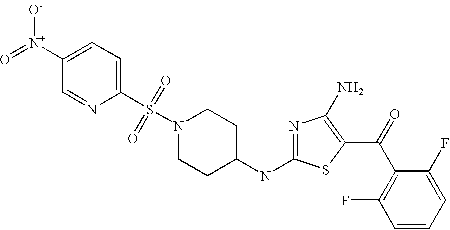 | 1 | 0.4 | NT | NT |
| F30 | 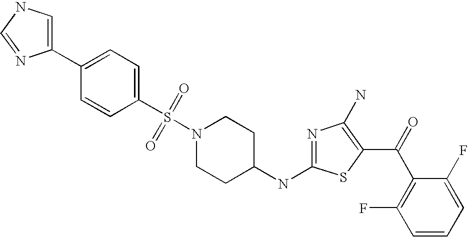 | 0.00025 | 0.00032 | 0.17 | 1.7 |
| F31 | 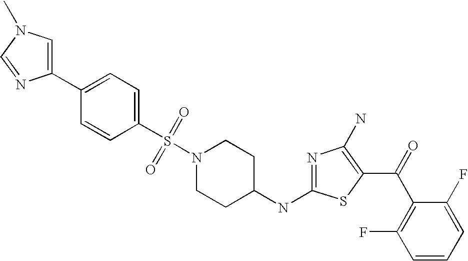 | <0.001 | 0.00055 | 0.08 | 0.3 |
| F32 | 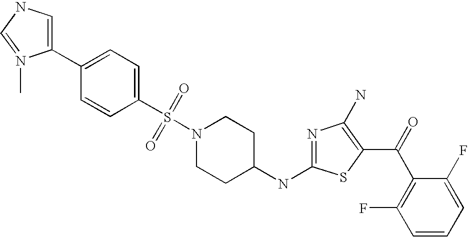 | 0.0004 | 0.0009 | 0.11 | 0.38 |
| F33 | 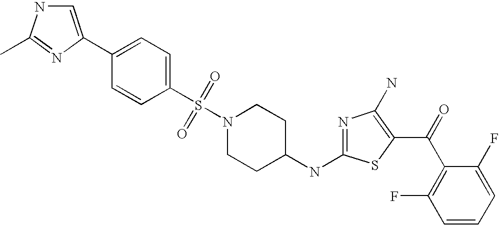 | 0.00028 | 0.00028 | 0.16 | 1.6 |
| F34 | 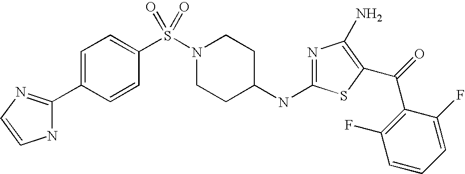 | <0.001 | 0.00051 | 1.5 | 2.6 |
| F35 | 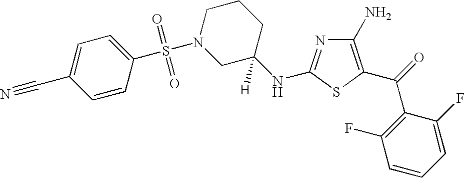 | 0.076 | 0.34 | 1.6 | 2.6 |
| F36 | 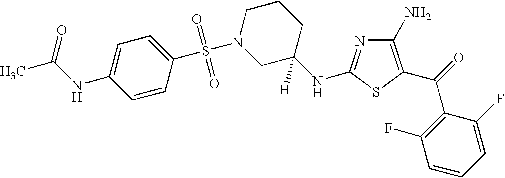 | 0.48 | 0.78 | >5 | >5 |
| F37 | 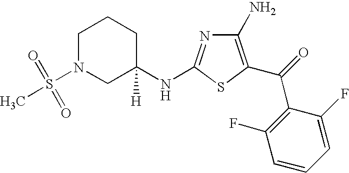 | 0.43 | 1.1 | 0.7 | 1.5 |
| F38 | 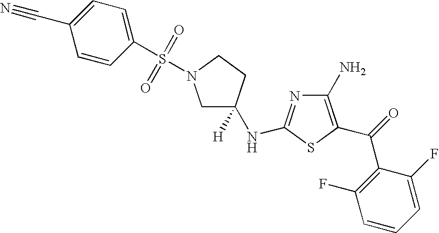 | 0.099 | 0.36 | NT | NT |
| F39 | 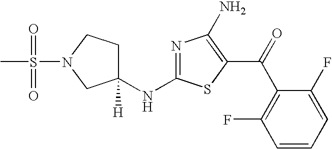 | 0.52 | 0.33 | NT | NT |
| F40 | 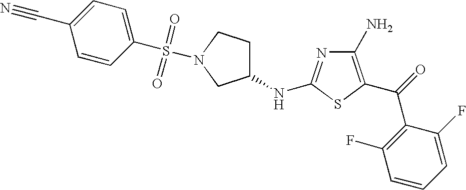 | 0.058 | 0.38 | NT | NT |
| F41 | 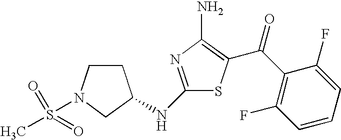 | 0.75 | 1.6 | NT | NT |
| F42 | 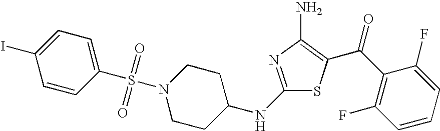 | NT | NT | NT | NT |
| F43 | 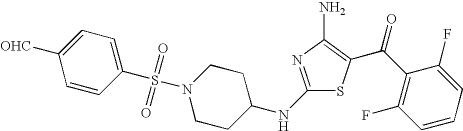 | NT | NT | NT | NT |
| F44 | 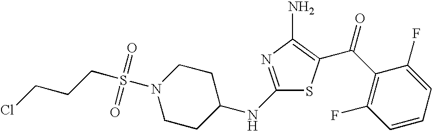 | NT | NT | NT | NT |
| F45 | 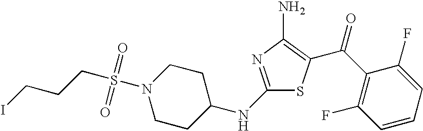 | NT | NT | NT | NT |
| F46 | 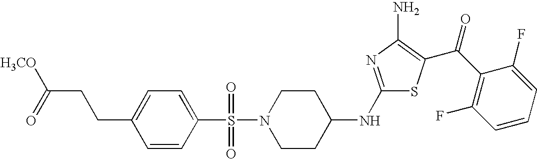 | 0.0008 | 0.002 | 0.79 | >5 |
| F47 | 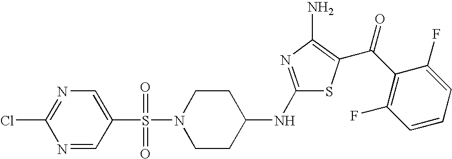 | 0.00027 | 0.00069 | 0.90 | 2.3 |
| F48 | 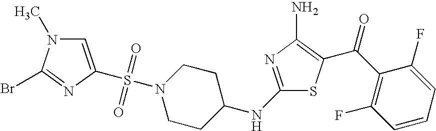 | 0.0076 | 37% @0.05 μM | 3.2 | 5 |
| F49 | 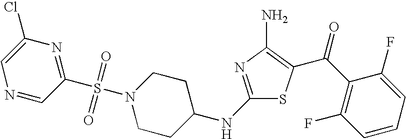 | 0.00046 | 0.0011 | NT | NT |
| F50 | 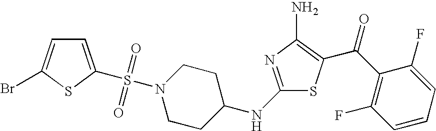 | 0.0011 | 0.0032 | 0.28 | 2.6 |
| F51 | 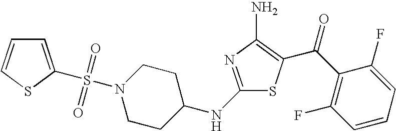 | 0.0015 | 0.0055 | 0.3 | 0.63 |
| F52 | 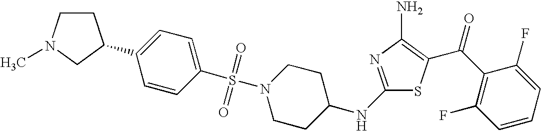 | 0.001 | 0.00052 | 0.093 | 0.5 |
| F53 | 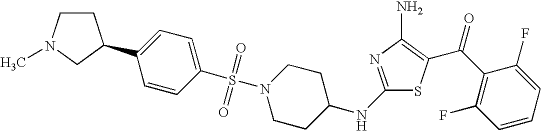 | 0.0013 | 0.00061 | 0.09 | 0.5 |
| G1 | 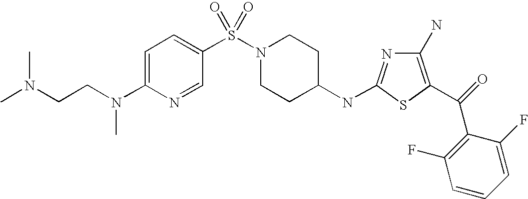 | 0.0014 | 0.00064 | 0.12 | >0.5 |
| G2 | 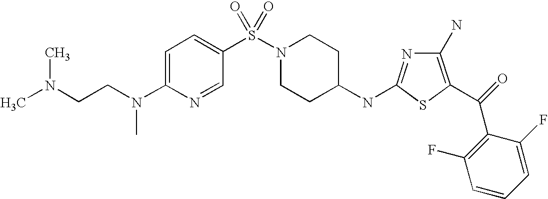 | 0.0012 | 0.00051 | 0.38 | 4 |
| G3 | 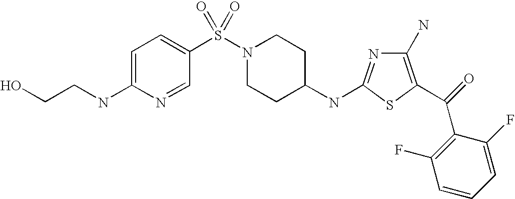 | <0.001 | 0.0012 | 1.7 | >5 |
| G4 | 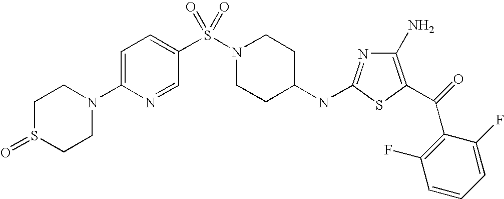 | 0.0014 | 0.00094 | 1.5 | 5 |
| G5 | 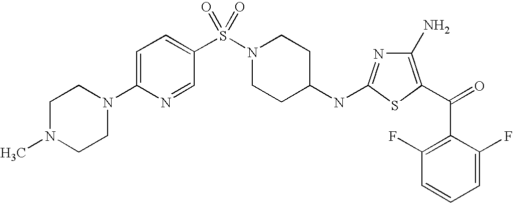 | 0.0013 | 0.0013 | 0.029 | >0.5 |
| G6 | 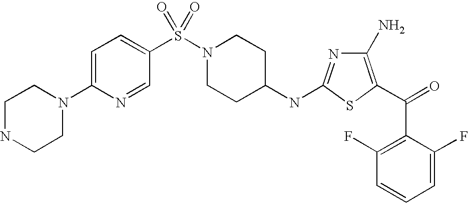 | 0.00069 | 0.00054 | 0.21 | 3.2 |
| G7 | 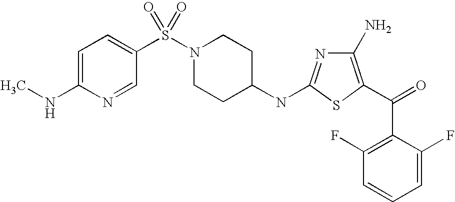 | 0.00075 | 0.0016 | 0.18 | 0.65 |
| G8 | 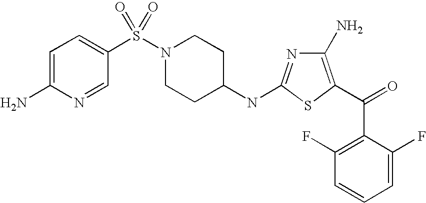 | 0.0006 | 0.0019 | 0.59 | 2.2 |
| G9 | 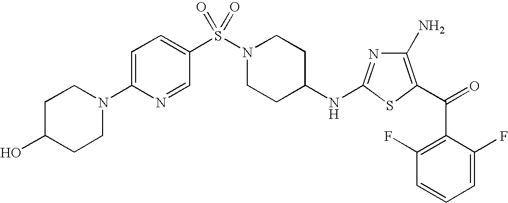 | 0.00052 | 0.0022 | 0.17 | 1.8 |
| G10 | 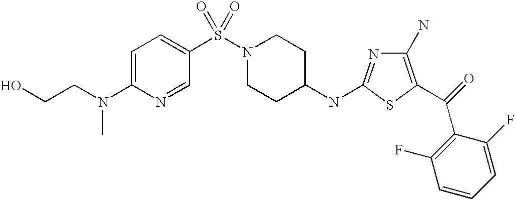 | <0.001 | 0.0012 | 0.67 | >5 |
| G11 | 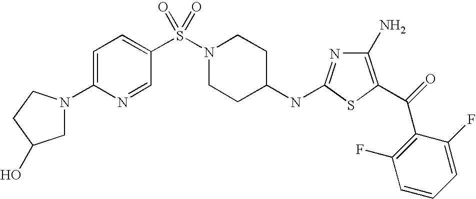 | <0.001 | 0.00086 | 0.42 | >5 |
| G12 | 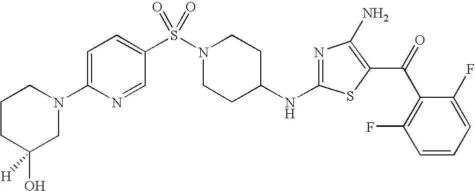 | 0.00049 | 0.0012 | 0.28 | >0.5 |
| G13 | 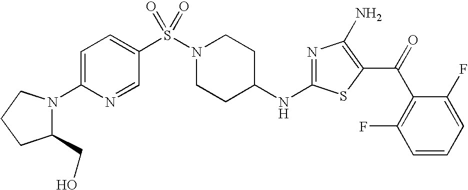 | <0.001 | 0.00064 | 0.17 | 3.9 |
| G14 | 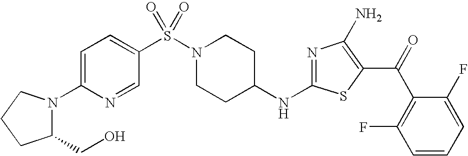 | 0.0005 | 0.0008 | 0.14 | 3.5 |
| G15 | 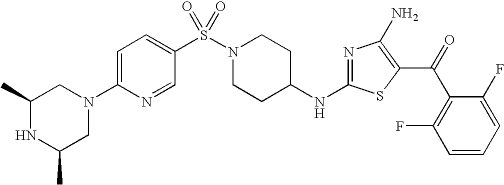 | 0.00073 | 0.00028 | 0.079 | >0.5 |
| G16 | 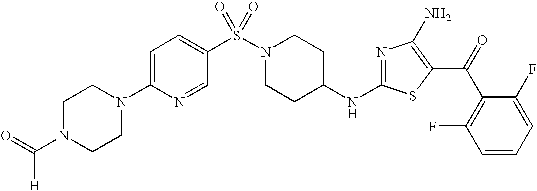 | 0.00051 | 0.00063 | 0.29 | >5 |
| G17 | 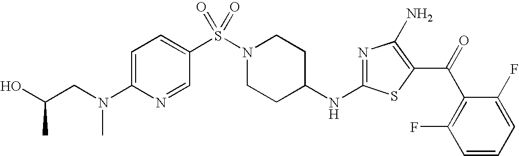 | 0.00055 | 0.0014 | 0.36 | 0.9 |
| G18 | 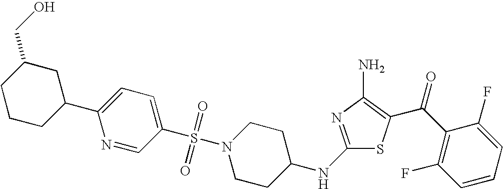 | 0.00039 | 0.00058 | 0.12 | 0.6 |
| G19 | 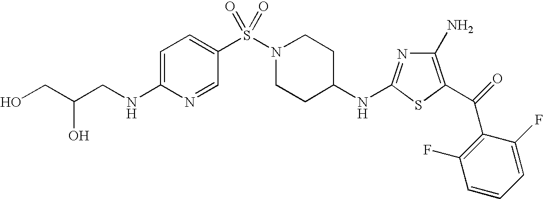 | 0.002 | 0.0034 | 4.1 | >5 |
| G20 | 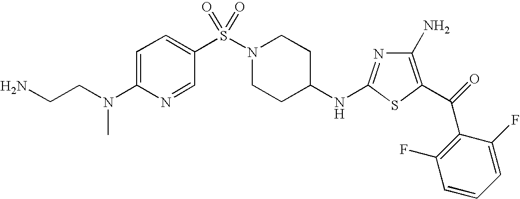 | 0.0049 | 0.0022 | 0.46 | 5 |
| G21 | 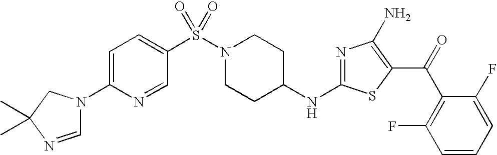 | <0.001 | 0.00068 | 1.9 | >5 |
| G22 | 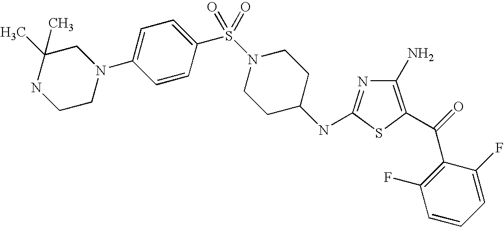 | 0.00066 | 0.00022 | 0.21 | 3 |
| G23 | 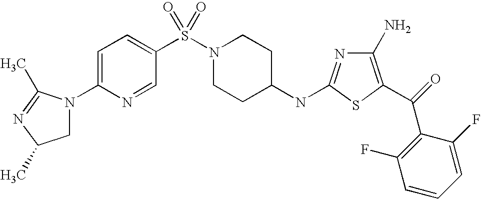 | <0.001 | 0.00044 | 0.75 | 5 |
| G24 | 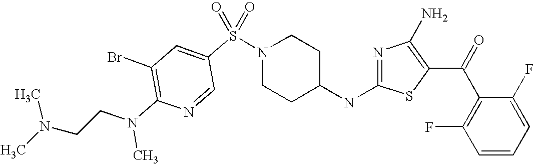 | 0.00085 | 0.00048 | 0.29 | 0.62 |
| G25 | 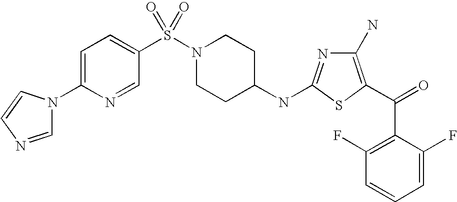 | 0.00027 | 0.00036 | 0.063 | >0.5 |
| G26 | 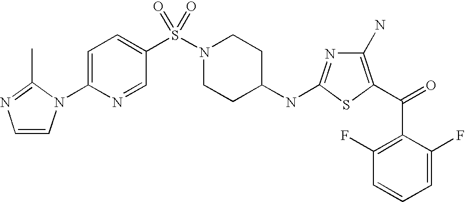 | 0.00064 | 0.0013 | 0.14 | 0.22 |
| G27 | 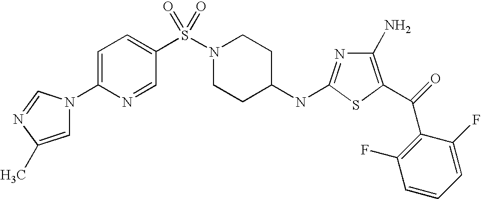 | 0.00041 | <0.001 | 0.057 | 0.25 |
| G28 | 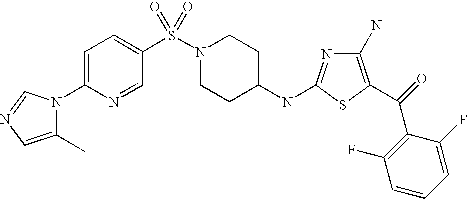 | 0.0004 | 0.00085 | 0.16 | 0.33 |
| G29 | 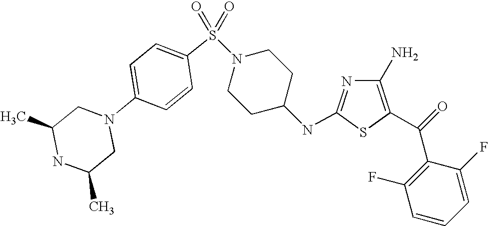 | 0.00072 | 0.00061 | 0.045 | 0.25 |
| G30 | 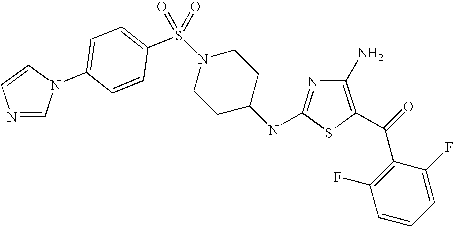 | 0.00031 | 0.00045 | NT | NT |
| G31 | 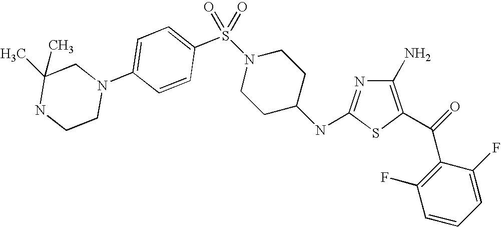 | 0.00082 | 0.00053 | 0.11 | 1.5 |
| G32 | 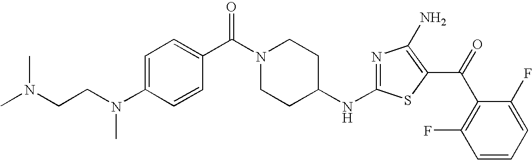 | 0.06 | 0.042 | 4.7 | >5 |
| G33 | 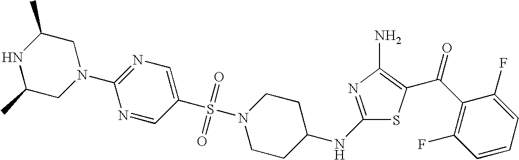 | 0.001 | 0.0003 | 0.051 | 0.8 |
| G34 | 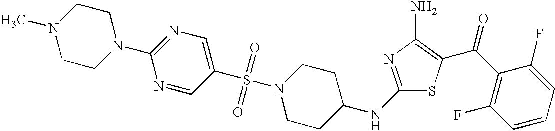 | 0.00082 | 0.00057 | 0.04 | 0.25 |
| H1 | 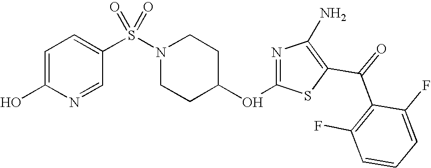 | <0.001 | 0.00072 | 2.6 | >5 |
| H2 | 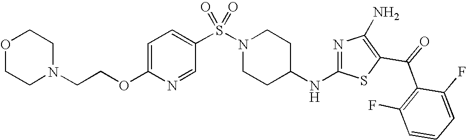 | 0.0028 | 0.00077 | 0.08 | 0.25 |
| H3 | 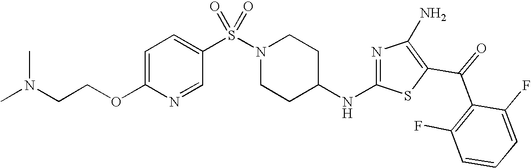 | 0.0018 | 0.00067 | 0.051 | 0.32 |
| H4 | 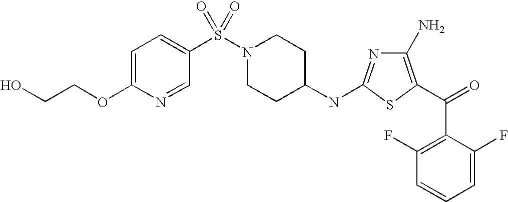 | 0.0007 | 0.0025 | 0.1 | 0.5 |
| H5 | 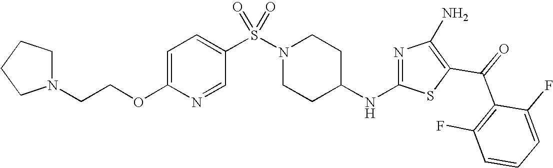 | 0.0011 | 0.00039 | 0.071 | >0.5 |
| H6 | 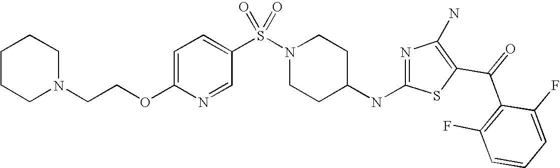 | 0.00084 | 0.00038 | 0.06 | 0.5 |
| H7 | 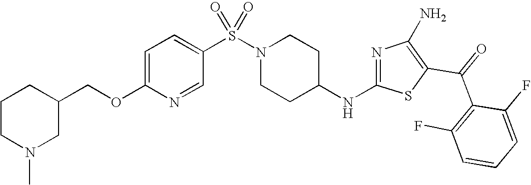 | 0.0008 | 0.00021 | 0.04 | 0.25 |
| H8 | 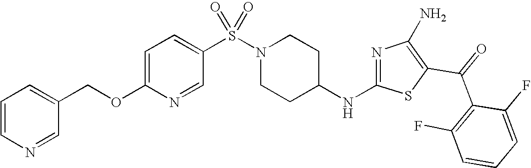 | <0.001 | 0.00067 | 0.58 | 1.3 |
| H9 | 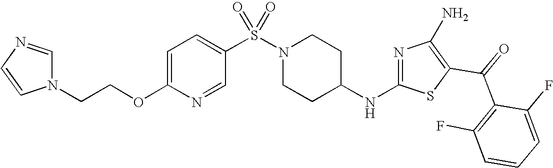 | <0.0005 | 0.0012 | 0.48 | 3.1 |
| H10 | 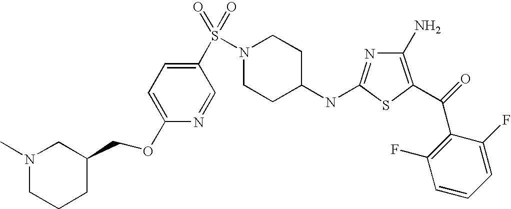 | 0.0011 | 0.0007 | 0.048 | >0.5 |
| H11 | 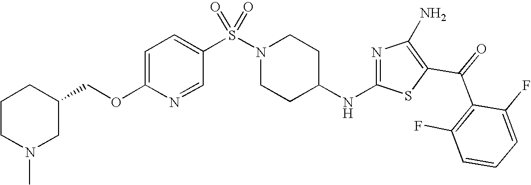 | 0.0069 | 0.00028 | 0.042 | 0.13 |
| H12 | 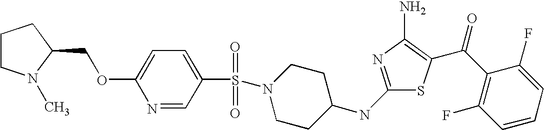 | 0.00088 | 0.00039 | 0.058 | 0.4 |
| H13 | 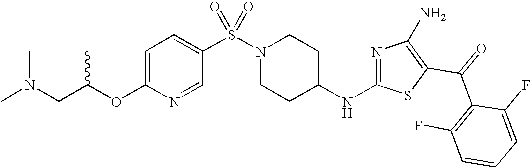 | 0.0011 | 0.00065 | 0.09 | 0.4 |
| H14 | 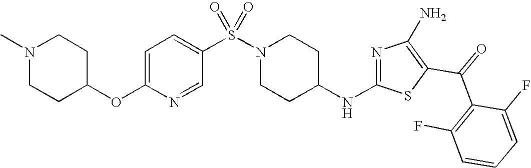 | 0.00074 | 0.00034 | 0.04 | 0.4 |
| H15 | 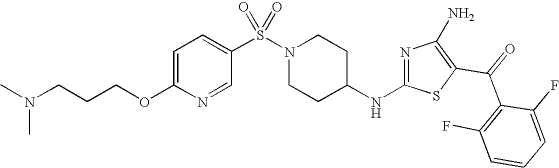 | 0.00064 | 0.00034 | 0.071 | 0.5 |
| H16 | 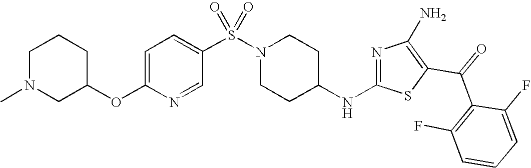 | 0.00048 | 0.00028 | 0.057 | 0.5 |
| H17 | 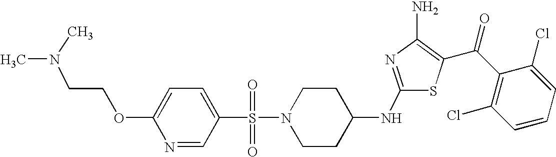 | 0.0018 | 0.0017 | 0.05 | 0.17 |
| H18 | 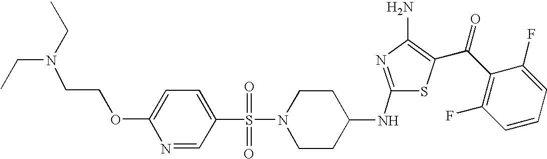 | 0.0016 | 0.0003 | 0.055 | >0.5 |
| H19 | 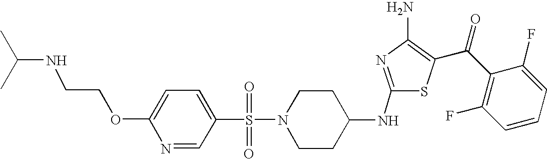 | 0.0015 | 0.00052 | 0.18 | 2.5 |
| H20 | 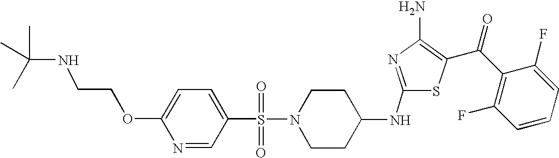 | 0.0015 | 0.00051 | 0.38 | 3 |
| H21 | 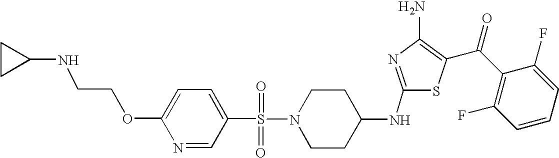 | 0.0015 | 0.00028 | 0.11 | 1.5 |
| H22 | 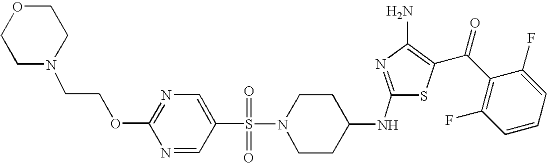 | 0.0012 | 0.0011 | 0.12 | 0.3 |
| H23 | 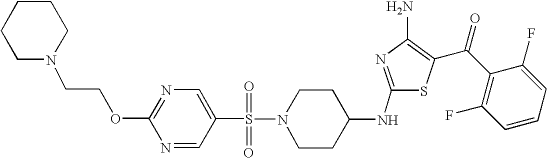 | 0.0015 | 0.00052 | 0.093 | 1.5 |
| H24 | 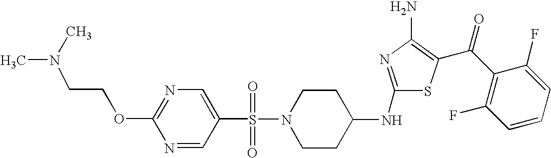 | 0.0018 | 0.00041 | 0.14 | 2 |
| H25 | 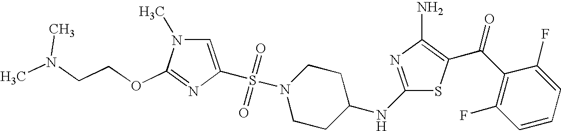 | 0.020 | 0.083 | >5 | >5 |
| H26 | 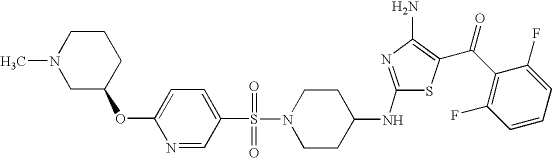 | 0.00037 | 0.0013 | 0.022 | 0.08 |
| H27 | 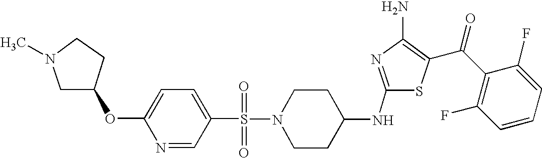 | 0.00035 | 0.00097 | 0.08 | 0.29 |
| H28 | 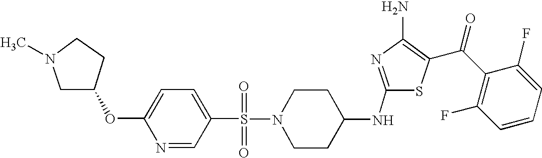 | 0.00041 | 0.001 | 0.049 | 0.16 |
| H29 | 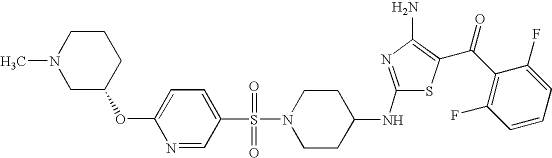 | 0.00052 | 0.0013 | 0.036 | 0.30 |
| I1 | 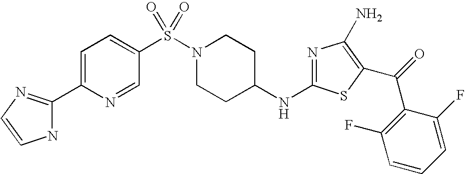 | 0.00026 | 0.00056 | 0.3 | 0.5 |
| I2 | 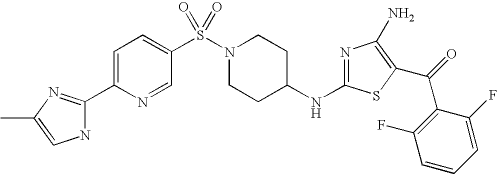 | 0.00041 | 0.00072 | 0.24 | 1.4 |
| I3 | 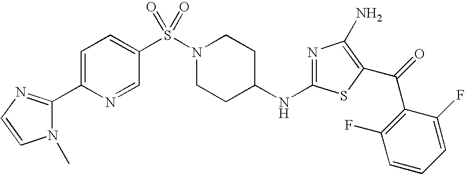 | 0.0017 | 0.002 | 0.16 | 0.5 |
| I4 | 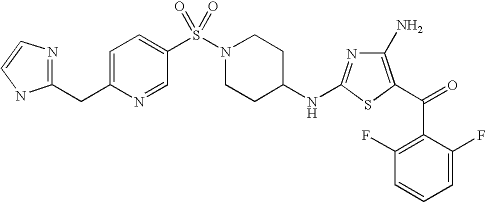 | <0.001 | 0.0018 | 1.9 | 4.7 |
| I5 | 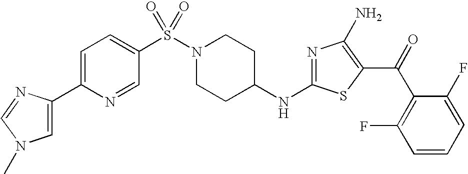 | 0.0051 | 0.00067 | 0.08 | 0.5 |
| I6 | 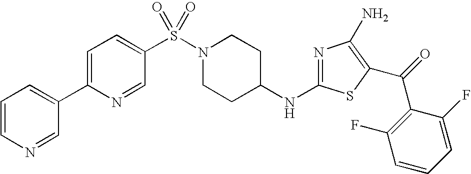 | 0.00032 | 0.00037 | 0.037 | 0.11 |
| I7 | 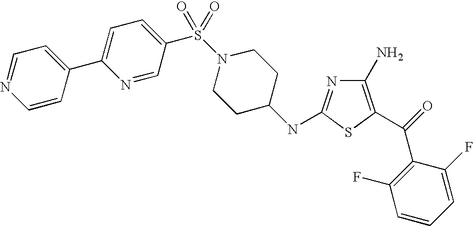 | <0.001 | 0.00038 | 1.3 | 5 |
| I8 | 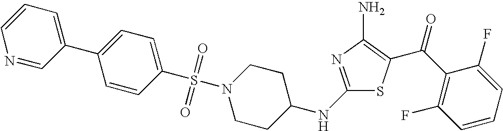 | 0.0003 | 0.00048 | 0.071 | 0.5 |
| I9 | 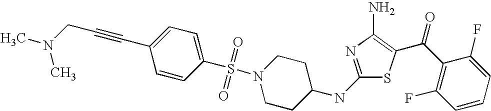 | NT | NT | 0.1 | 0.5 |
| I10 | 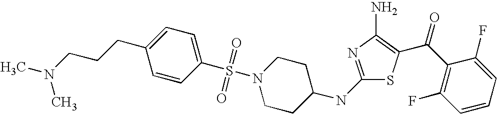 | 0.0012 | 0.00068 | 0.2 | 1.9 |
| I11 | 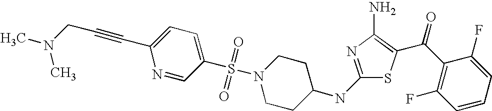 | <0.0005 | 0.0005 | 0.016 | 0.025 |
| I12 | 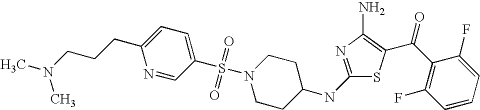 | 0.0013 | 0.0011 | 0.3 | 3.4 |
| I13 | 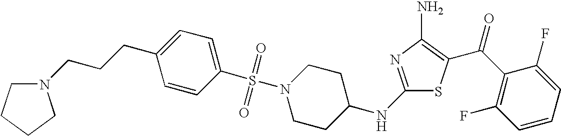 | 0.0013 | 0.00045 | 0.28 | 0.6 |
| I14 | 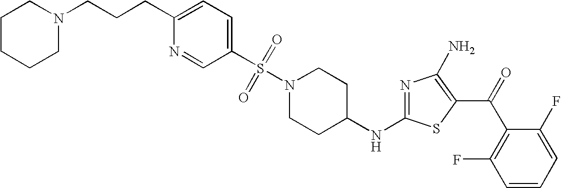 | 0.0012 | 0.00057 | 0.14 | 0.3 |
| J1 | 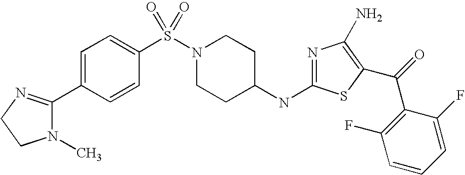 | <0.001 | 0.00063 | 1.2 | 5 |
| J2 | 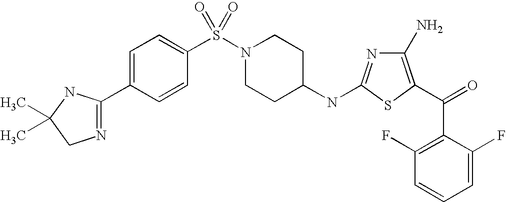 | <0.001 | 0.00018 | 0.5 | 1.8 |
| J3 | 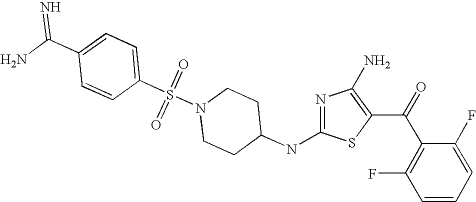 | <0.001 | 0.00025 | >5 | >5 |
| J4 | 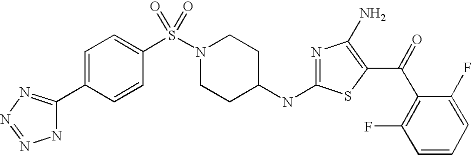 | <0.001 | 0.0017 | >5 | >5 |
| J5 | 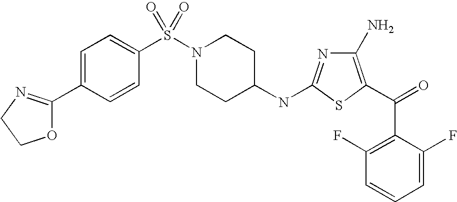 | 0.0028 | 0.0039 | 0.21 | 0.48 |
| J6 | 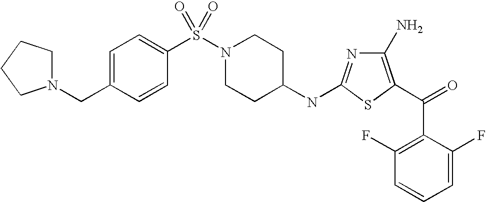 | <0.001 | 0.00058 | 0.39 | 2 |
| J7 | 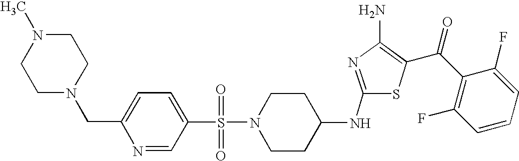 | 0.0019 | 0.00078 | 0.13 | 0.3 |
| J8 | 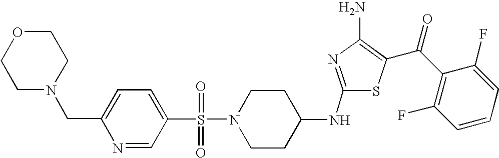 | 0.0013 | 0.0012 | 0.098 | 0.4 |
| J9 | 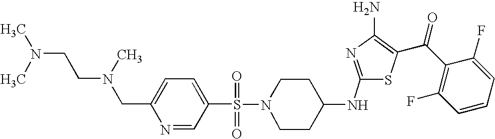 | 0.0024 | 0.00085 | 0.13 | 0.32 |
| J10 | 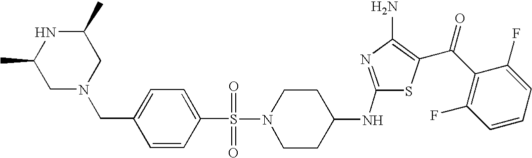 | 0.0017 | 0.00039 | 0.16 | 2.9 |
| K1 | 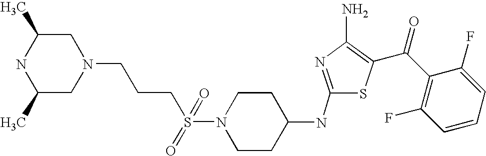 | 0.0078 | 0.002 | >5 | >5 |
| K2 | 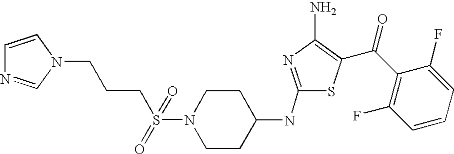 | 0.0063 | 0.0047 | 2.3 | >5 |
| K3 | 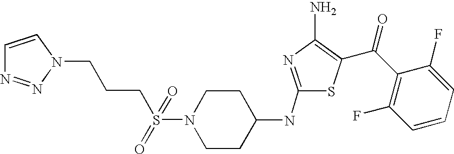 | 0.0044 | 0.004 | >0.5 | >0.5 |
| K4 | 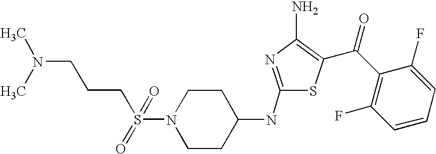 | 0.0018 | 0.0013 | 0.41 | 1.5 |
| K5 | 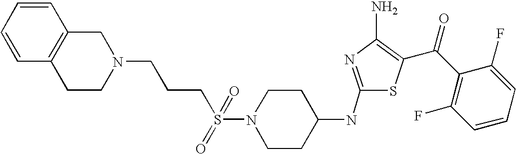 | 0.001 | 0.0015 | 0.14 | 0.58 |
| K6 | 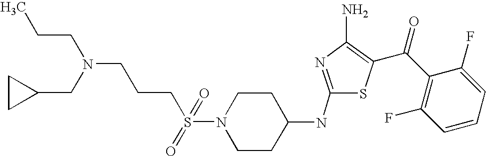 | 0.0058 | 0.0015 | 0.18 | 0.8 |
| K7 | 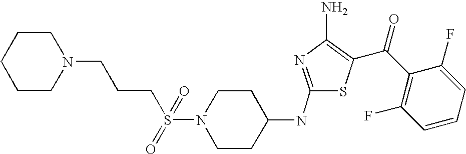 | 0.002 | 0.0029 | 0.21 | 1.7 |
| K8 | 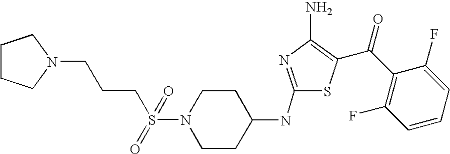 | 0.0018 | 0.0027 | 0.31 | 2.9 |
| K9 | 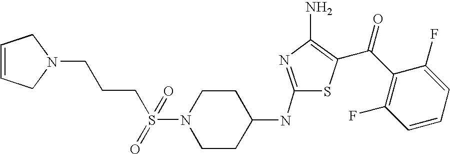 | <0.0013 | 0.0016 | 0.09 | 0.93 |
| K10 | 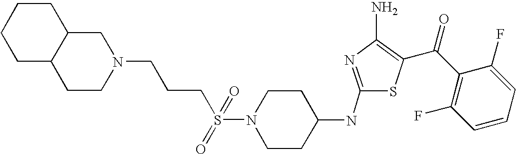 | 0.0026 | 0.0011 | 0.19 | 1.3 |
| K11 | 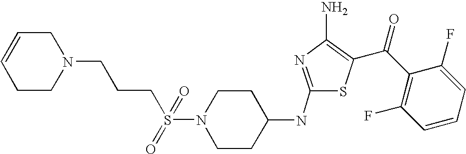 | 0.0029 | 0.0018 | 0.13 | 1.3 |
| K12 | 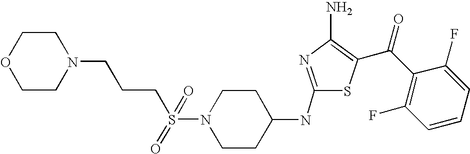 | 0.0067 | 0.0047 | 0.6 | 5 |
| K13 | 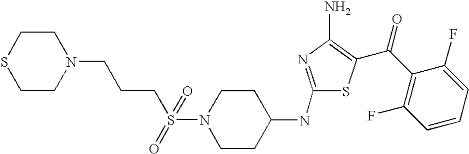 | 0.0039 | 0.0025 | 0.39 | 1.3 |
| K14 | 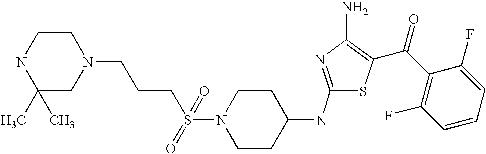 | 0.0079 | 0.0029 | 3.3 | >5 |
| K15 | 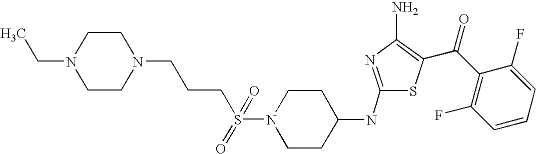 | 0.0087 | 0.0025 | 1.3 | 5 |
| K16 | 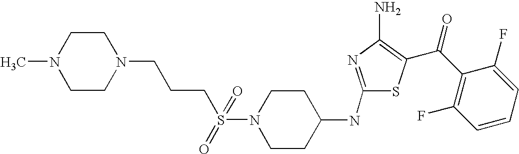 | 0.0078 | 0.0028 | 1.8 | 5 |
| K17 | 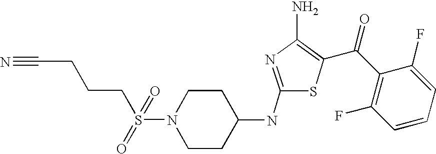 | 0.0025 | 0.0034 | 0.89 | 2.2 |
| K18 | 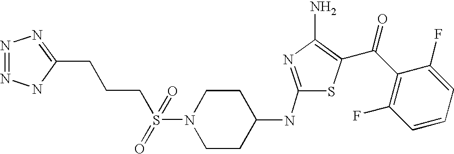 | 0.0031 | 0.018 | >5 | >5 |
| K19 | 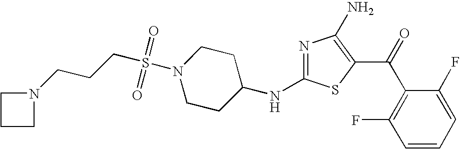 | 0.0013 | 0.002 | 0.81 | >5 |
| K20 | 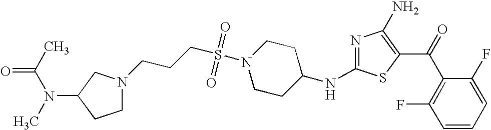 | 0.0048 | 0.0015 | 2 | 5 |
| K21 | 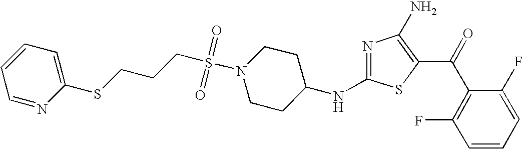 | 0.0027 | 0.0044 | >0.5 | >0.5 |
| K22 | 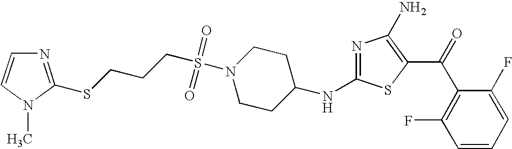 | 0.0048 | 0.0073 | >0.5 | >0.5 |
| K23 | 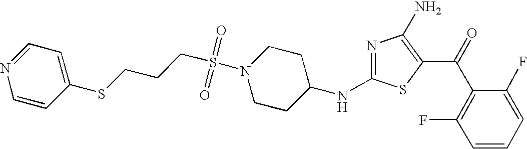 | 0.0028 | 0.003 | 0.46 | >0.5 |
| K24 | 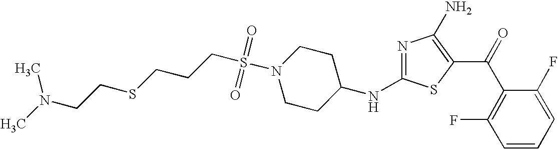 | 0.0059 | 0.002 | >0.05 | >0.05 |
| K25 | 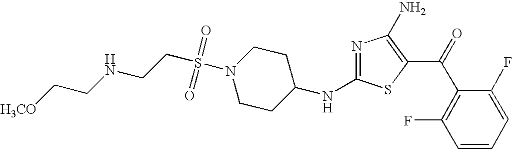 | 0.0044 | 0.0014 | 0.88 | 2.6 |
| K26 | 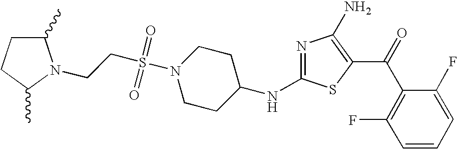 | 0.013 | 0.0021 | 0.19 | 0.80 |
| K27 | 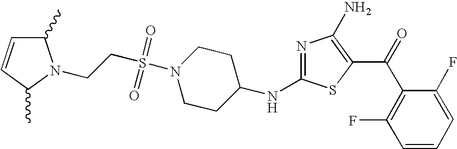 | 0.011 | 0.0035 | 0.23 | 0.80 |
| K28 | 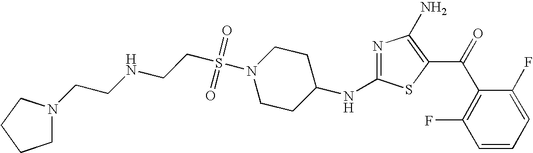 | 0.010 | 0.0028 | >5 | >5 |
| K29 | 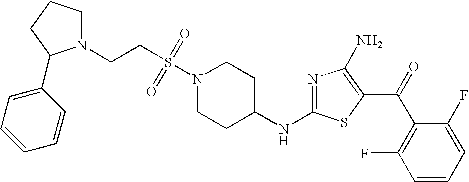 | 0.0037 | 0.0016 | 0.16 | 0.51 |
| K30 | 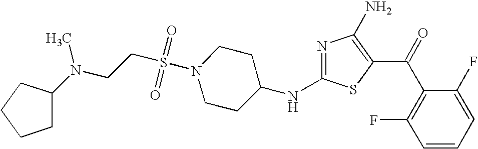 | 0.0094 | 0.0024 | 0.19 | 0.62 |
| K31 | 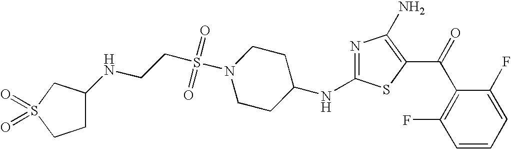 | 0.0055 | 0.0043 | >5 | >5 |
| K32 | 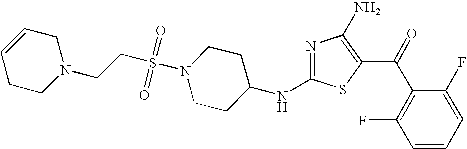 | 0.0062 | 0.0021 | 0.19 | 1.4 |
| K33 | 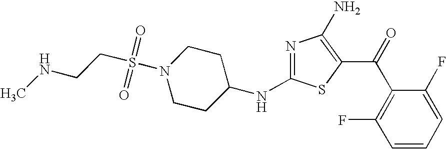 | 0.0056 | 0.00064 | 0.65 | 5 |
| K34 | 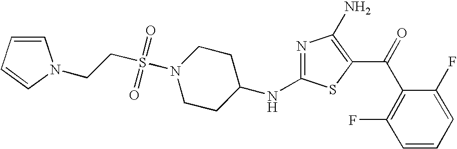 | 0.006 | 0.0054 | 0.28 | 1.2 |
| K35 | 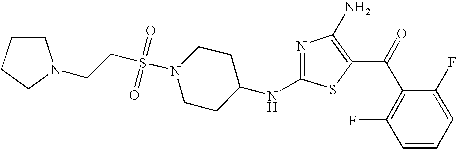 | 0.003 | 0.0011 | 0.14 | 0.45 |
| K36 | 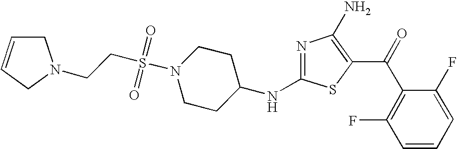 | 0.0075 | 0.0066 | 0.65 | 1.9 |
| K37 | 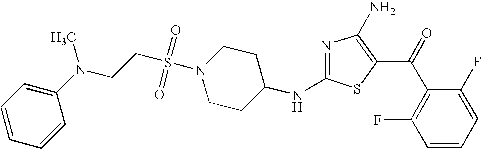 | 0.007 | 0.0032 | 0.31 | 1.3 |
| K38 | 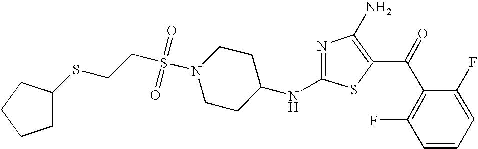 | 0.0079 | 0.0064 | 0.46 | 3 |
| K39 | 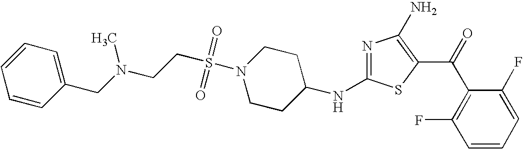 | 0.0014 | 0.0018 | 0.044 | 0.17 |
| K40 | 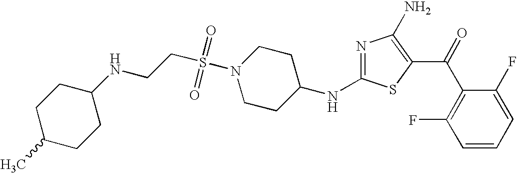 | 0.00051 | 0.0021 | 0.066 | 0.28 |
| K41 | 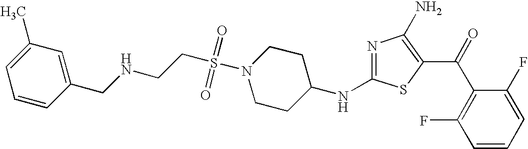 | 0.00063 | 0.0019 | 0.07 | 0.17 |
| K42 | 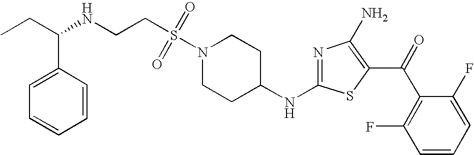 | 0.001 | 0.0019 | 0.05 | 0.15 |
| K43 | 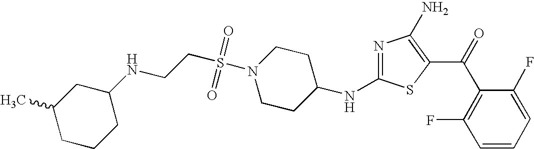 | 0.00057 | 0.0024 | 0.051 | 0.3 |
| K44 | 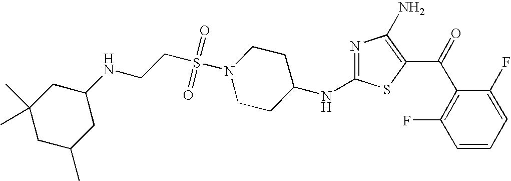 | 0.00049 | 0.0025 | 0.10 | 0.36 |
| K45 | 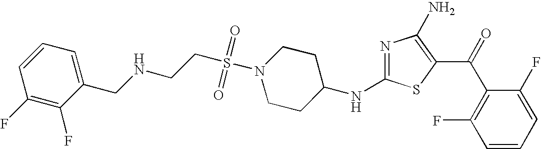 | 0.00082 | 0.001 | 0.050 | 0.17 |
| K46 | 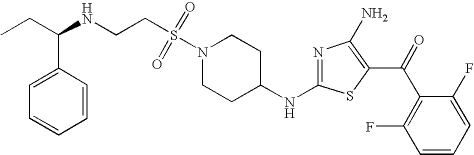 | 0.00081 | 0.0021 | 0.13 | 0.26 |
| K47 | 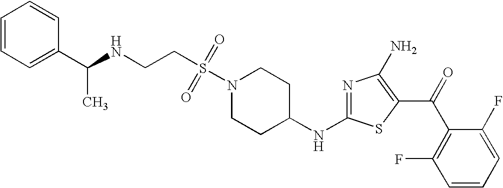 | 1.2 | 1.2 | 0.10 | 0.25 |
| K48 | 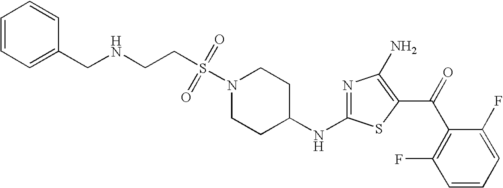 | 1 | 1.3 | 0.17 | 0.5 |
| K49 | 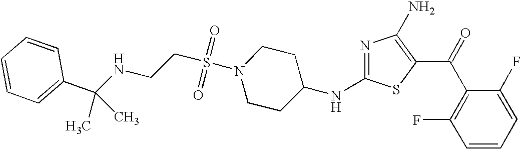 | 1.1 | 2 | 0.18 | 0.5 |
| K50 | 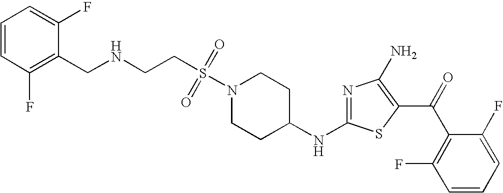 | 2.1 | 1.6 | 0.2 | 0.5 |
| K51 | 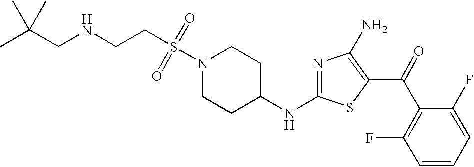 | 1.1 | 2.7 | 0.17 | 0.5 |
| K52 | 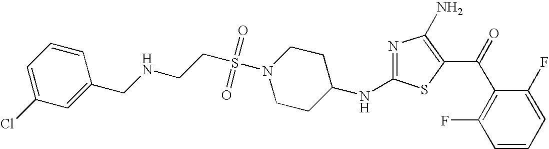 | 3.7 | 3.7 | 0.11 | 0.25 |
| K53 | 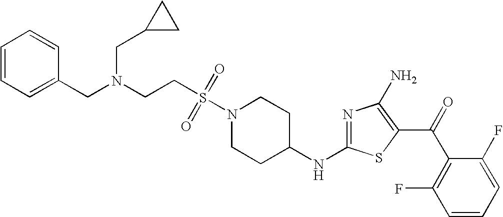 | 0.55 | 1 | 0.23 | 0.56 |
| K54 | 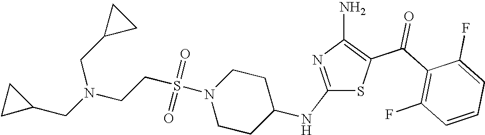 | 0.005 | 0.0013 | 0.28 | 0.8 |
| K55 | 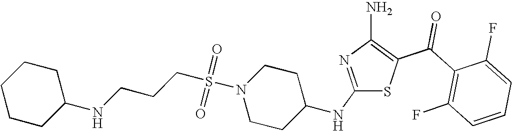 | 0.008 | NT | 0.68 | NT |
| K56 | 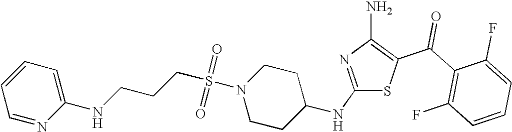 | 0.008 | NT | 2.64 | NT |
| K57 | 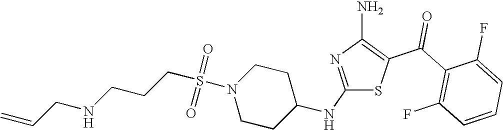 | 0.007 | NT | 0.48 | NT |
| K58 | 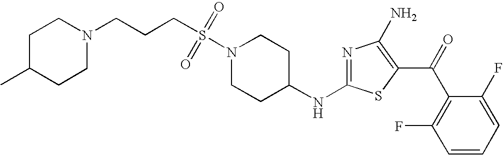 | 0.011 | NT | 0.15 | NT |
| K59 | 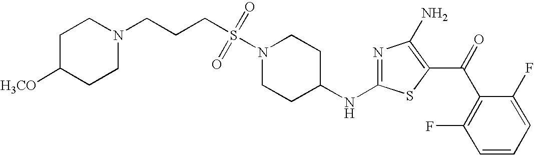 | 0.011 | NT | 0.27 | NT |
| K60 | 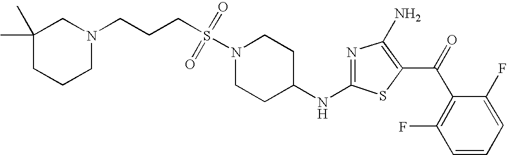 | 0.013 | NT | 0.12 | NT |
| K61 | 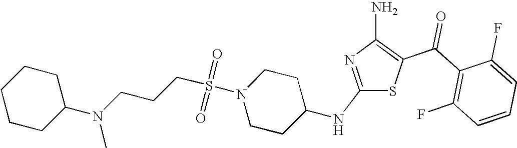 | 0.008 | NT | 0.17 | ND |
| L1 | 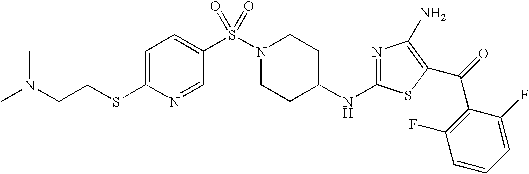 | 0.00062 | 0.0003 | 0.078 | >0.5 |
| L2 | 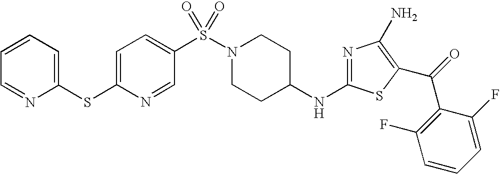 | 0.0015 | 0.0027 | >0.5 | >0.5 |
| L3 | 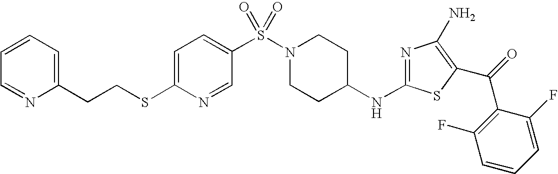 | 0.00068 | 0.0012 | 0.35 | >0.5 |
| L4 | 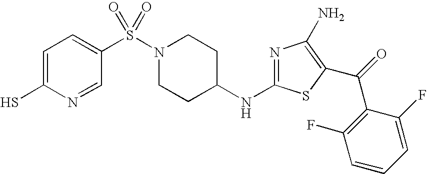 | 0.0003 | 0.0018 | >0.5 | >0.5 |
| L5 | 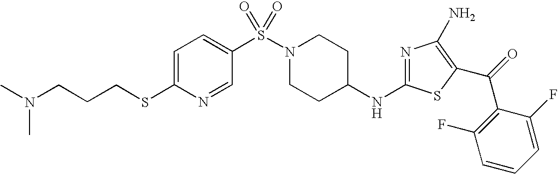 | 0.0015 | 0.00067 | 0.07 | >0.5 |
| L6 | 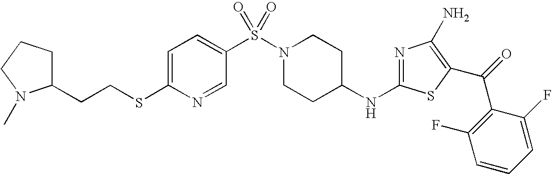 | 0.0015 | 0.00095 | 0.075 | 0.3 |
| L7 | 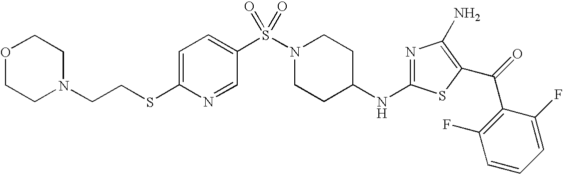 | 0.0015 | 0.0022 | 0.095 | 0.3 |
| M1 | 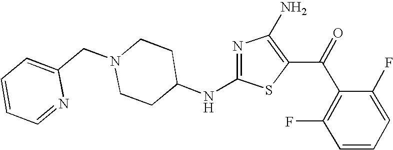 | >0.500 | 0.240 | 2.8 | 5 |
| M2 | 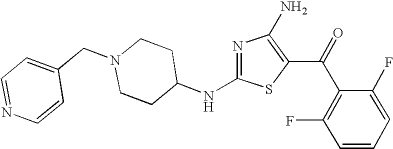 | 0.433 | 0.0335 | 2.1 | 5 |
| N1 | 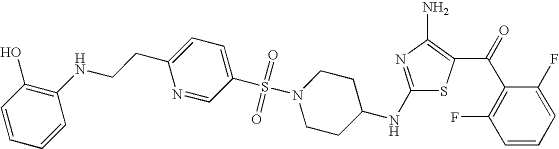 | 0.00028 | 0.00049 | 0.86 | 1.6 |
| N2 | 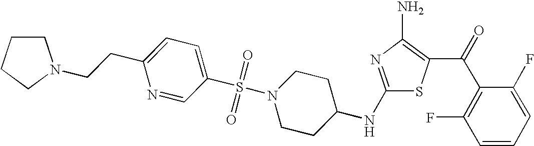 | 0.0012 | 0.00049 | 0.23 | >0.5 |
| N3 | 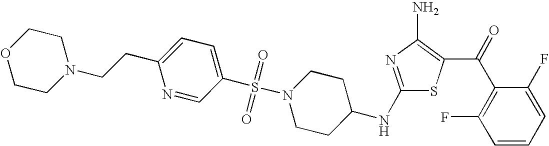 | 0.0011 | 0.00076 | 0.17 | >0.5 |
| N4 | 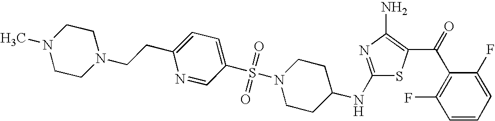 | 0.0017 | 0.00092 | 0.36 | >0.5 |
| N5 | 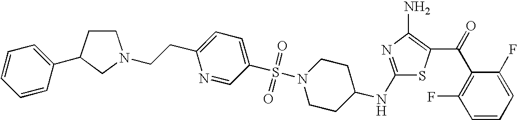 | 0.0018 | 0.0015 | 0.18 | >0.5 |
| N6 | 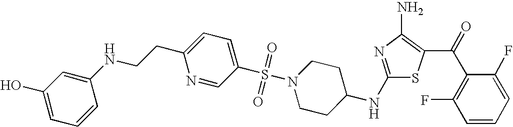 | 0.0003 | 0.00031 | 0.41 | 1.3 |
| N7 | 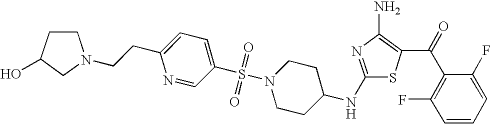 | 0.00093 | 0.00035 | 0.89 | 4 |
| N8 | 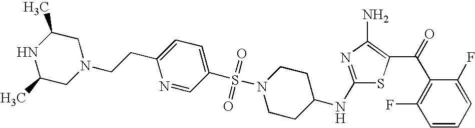 | 0.0011 | 0.00032 | 1.3 | 5 |
| N9 | 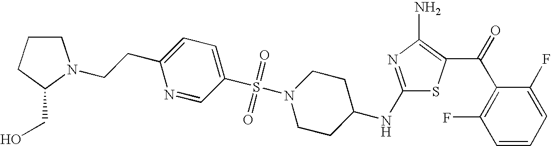 | 0.0008 | 0.00026 | 0.07 | 0.7 |
| N10 | 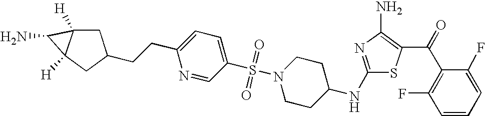 | 0.0013 | 0.00021 | 0.38 | 3 |
| N11 | 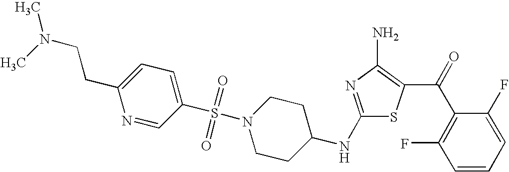 | 0.0016 | 0.00039 | 0.14 | 1.6 |
| N12 | 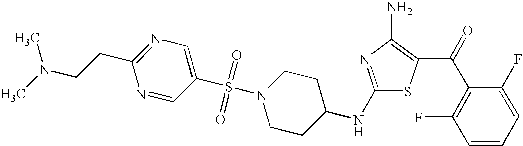 | 0.0017 | 0.00062 | 0.067 | 0.13 |
| T1 | 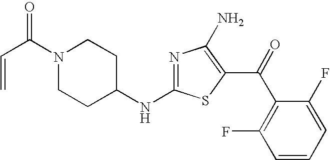 | NT | NT | NT | NT |
| U1 | 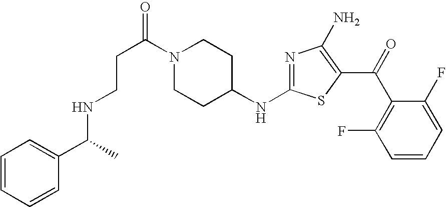 | 0.0207 | NT | 0.91 | ND |
| U4 | 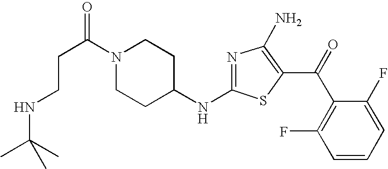 | 40% @0.5 μM | NT | >5 | ND |
| V177 | 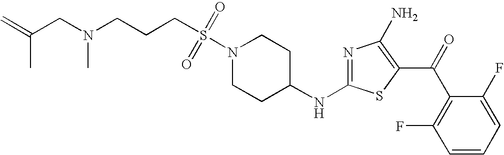 | 0.017 | NT | 0.09 | NT |
| V178 | 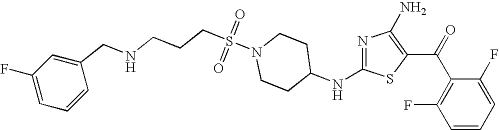 | 0.0046 | NT | 0.08 | NT |
| V179 | 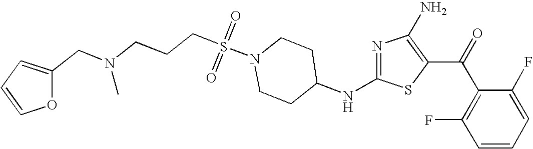 | 0.011 | NT | 0.17 | NT |
| V180 | 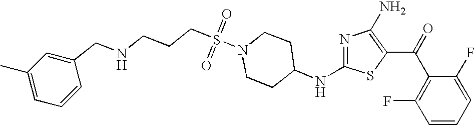 | 0.0040 | NT | 0.09 | NT |
| V181 | 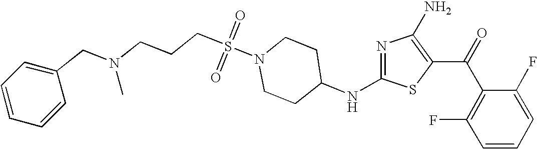 | 0.0065 | NT | 0.06 | NT |
| V182 | 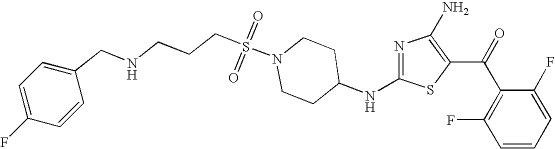 | 0.0047 | NT | 0.07 | NT |
| V183 | 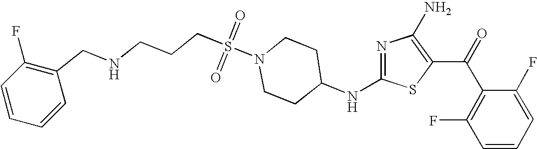 | 0.0040 | NT | 0.09 | NT |
| V184 | 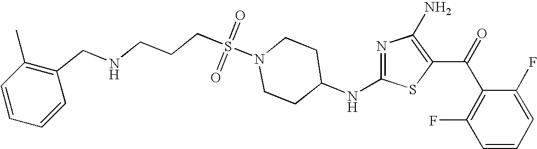 | 0.0036 | NT | 0.06 | NT |
| V185 | 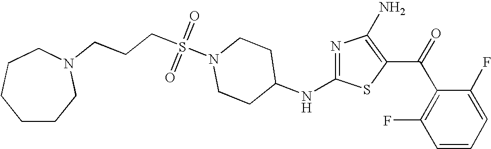 | NT | NT | NT | NT |

TABLE 2
(I)

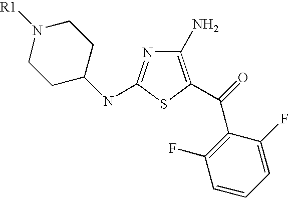

| | | CDK2 | CDK4 | HCT-116 |
|---|---|---|---|---|
| | | % Inhibition | % Inhibition | % Inhibition |
| Example | R¹ | At 0.03 μM | at 0.03 μM | at 0.25 μM |
| O1 | 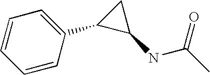 | 30 | −4 | 0 |
| O2 | 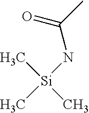 | 34 | 6 | 0 |
| O3 | 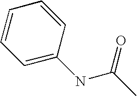 | 34 | 6 | 0 |
| O4 | 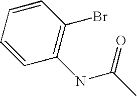 | 27 | 10 | 4 |
| O5 | 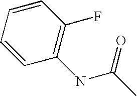 | 35 | 3 | 31 |
| O6 | 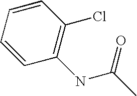 | 36 | 10 | 12 |
| O7 | 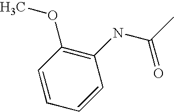 | 40 | 10 | 43 |
| O8 | 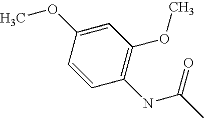 | 37 | 15 | 0 |
| O9 | 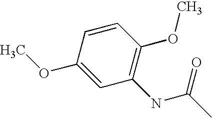 | 35 | 2 | 13 |
| O10 | 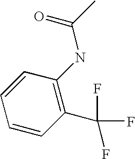 | 28 | 5 | 0 |
| O11 | 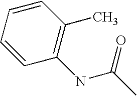 | 35 | 6 | 0 |
| O12 | 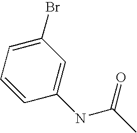 | 31 | 3 | 25 |
| O13 | 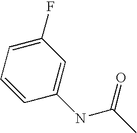 | 37 | 8 | 22 |
| O14 | 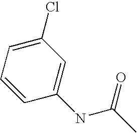 | 36 | 9 | 23 |
| O15 | 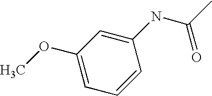 | 36 | 4 | 13 |
| O16 | 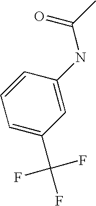 | 34 | 5 | 6 |
| O17 | 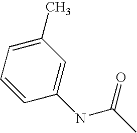 | 32 | 6 | 8 |
| O18 | 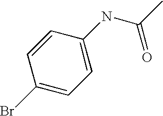 | 27 | 9 | 20 |
| O19 | 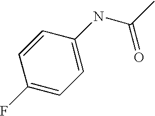 | 31 | 9 | 7 |
| O20 | 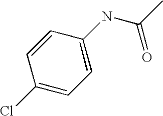 | 26 | 7 | 15 |
| O21 | 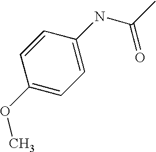 | 37 | 13 | 21 |
| O22 | 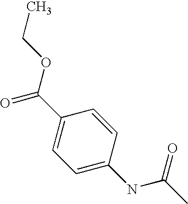 | 34 | 13 | 25 |
| O23 | 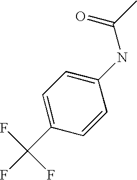 | 36 | 10 | 24 |
| O24 | 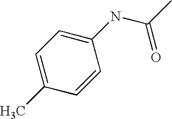 | 34 | 21 | 6 |
| O25 | 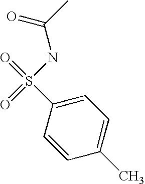 | 31 | 3 | 27 |
| O26 | 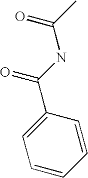 | 33 | 10 | 12 |
| O27 | 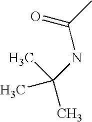 | 38 | 9 | 24 |
| O28 | 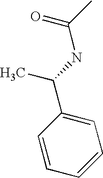 | 27 | 12 | 43 |
| O29 | 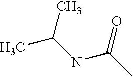 | 30 | 10 | 33 |
| O30 | 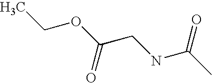 | 27 | 16 | 31 |
| O31 | 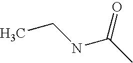 | 33 | 6 | 37 |
| O32 | 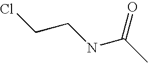 | 30 | 12 | 99 |
| O33 | 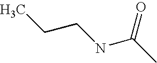 | 30 | −3 | 31 |
| O34 | 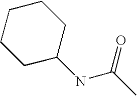 | 30 | 6 | 22 |
| O35 | 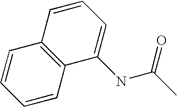 | 29 | 5 | 0 |
| O36 | 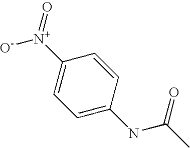 | 23 | 12 | 28 |
| O37 | 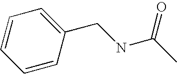 | 40 | 12 | 34 |
| O38 | 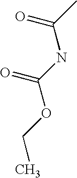 | 29 | 15 | 29 |
| O39 | 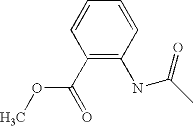 | 32 | 13 | 27 |
| O40 | 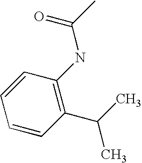 | 30 | 6 | 3 |
| O41 | 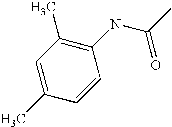 | 33 | 1 | 26 |
| O42 | 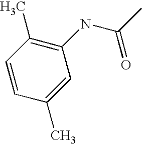 | 35 | 10 | 26 |
| O43 | 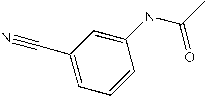 | 31 | 10 | 12 |
| O44 | 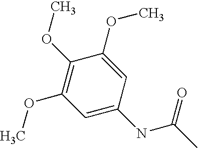 | 22 | 12 | 29 |
| O45 | 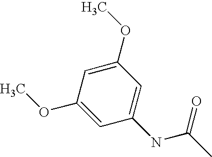 | 35 | 17 | 32 |
| O46 | 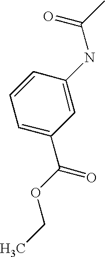 | 29 | 15 | 41 |
| O47 | 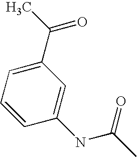 | 35 | 14 | 35 |
| O48 | 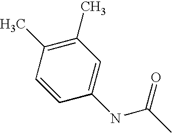 | 28 | 11 | 16 |
| O49 | 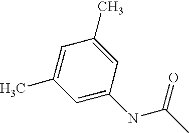 | 33 | −1 | 20 |
| O50 | 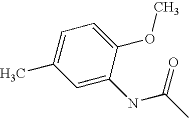 | 37 | 13 | 62 |
| O51 | 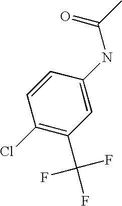 | 30 | 7 | 11 |
| O52 | 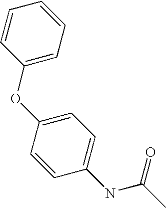 | 24 | 11 | 33 |
| O53 | 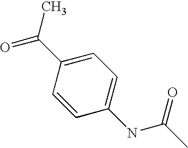 | 30 | 11 | 41 |
| O54 | 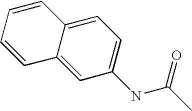 | 34 | 15 | 46 |
| O55 | 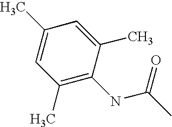 | 28 | 10 | 41 |
| O56 | 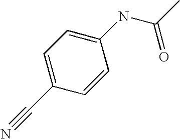 | 29 | 9 | 37 |
| O57 | 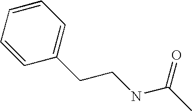 | 28 | −2 | 41 |
| O58 | 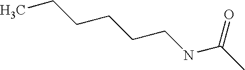 | 34 | 6 | 42 |
| O59 | 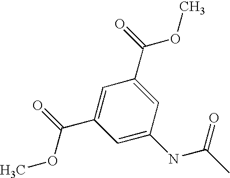 | 28 | 7 | 32 |
| O60 | 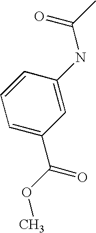 | 24 | 12 | 39 |
| O61 | 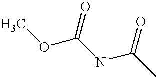 | 33 | 12 | 38 |
| O62 | 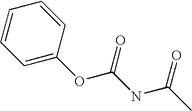 | 36 | 18 | 41 |
| O63 | 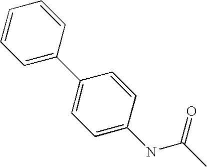 | 28 | 4 | 40 |
| O64 | 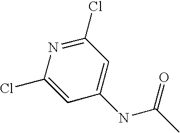 | 32 | 7 | 37 |
| O65 | 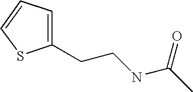 | 22 | −7 | 44 |
| O66 | 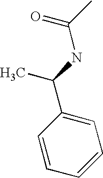 | 32 | −1 | 36 |
| O67 | 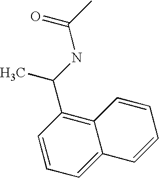 | 31 | 8 | 50 |
| O68 | 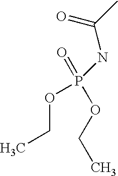 | 24 | 6 | 45 |
| O69 | 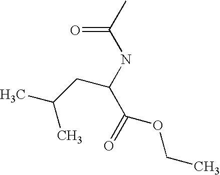 | 29 | 5 | 51 |
| O70 | 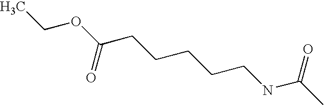 | 28 | 7 | 52 |
| O71 | 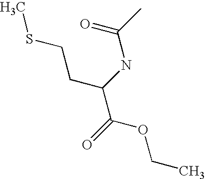 | 30 | 7 | 51 |
| O72 | 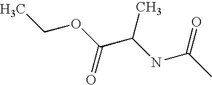 | 24 | 11 | 62 |
| O73 | 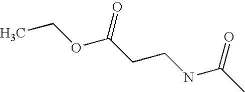 | 29 | 4 | 42 |
| O74 | 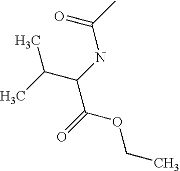 | 35 | 102 | 34 |
| O75 | 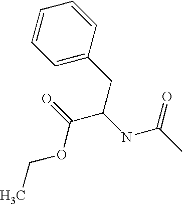 | 25 | 10 | 41 |
| O76 | 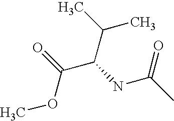 | 22 | 5 | 49 |
| O77 | 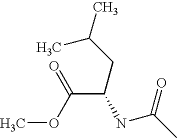 | 24 | 8 | 43 |
| O78 | 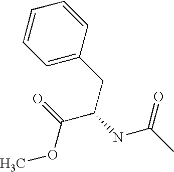 | 25 | 14 | 47 |
| O79 | 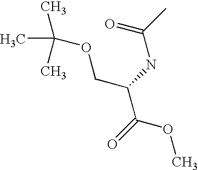 | 32 | 8 | 49 |
| O80 | 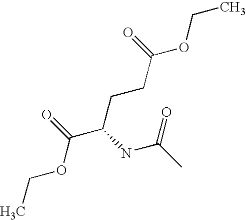 | 23 | 15 | 46 |
| O81 | 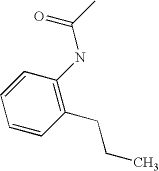 | 25 | −4 | 44 |
| O82 | 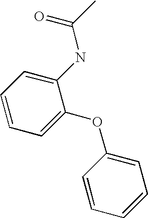 | 34 | 4 | 34 |
| O83 | 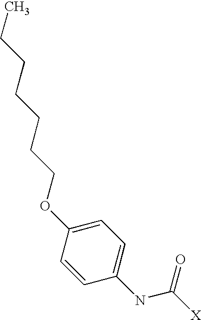 | 29 | 12 | 59 |
| O84 | 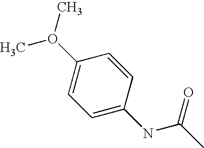 | 23 | 14 | 42 |
| O85 | 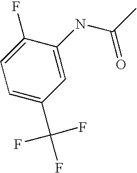 | 34 | 8 | 47 |
| O86 | 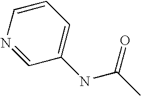 | 32 | 23 | 47 |
| O87 | 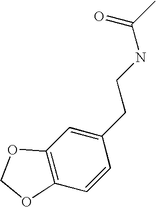 | 25 | 16 | 44 |
| O88 | 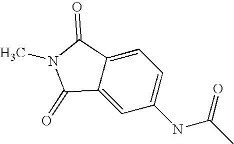 | 31 | 12 | 45 |

TABLE 3
(I)

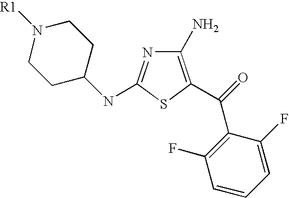

| | | CDK2 | CDK4 | HCT-116 | HCT-116 |
|---|---|---|---|---|---|
| | | % Inhibition | % Inhibition | % Inhibition | % Inhibition |
| Example | R1 | at 0.03 μM | at 0.03 μM | at 0.1 μM | at 0.25 μM |
| P1 | 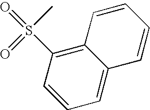 | 45 | 51 | 9 | 32 |
| P2 | 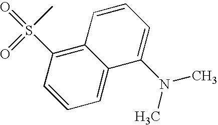 | 43 | 69 | 15 | 24 |
| P3 | 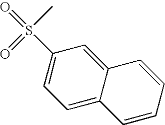 | 64 | 65 | 17 | 23 |
| P4 | 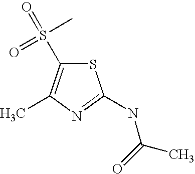 | 15 | 32 | 18 | 24 |
| P5 | 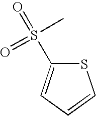 | 64 | 70 | 27 | 32 |
| P6 | 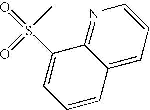 | −32 | 18 | 22 | 23 |
| P7 | 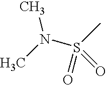 | 49 | 47 | 25 | 23 |
| P8 | 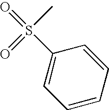 | 73 | 72 | 37 | 33 |
| P9 | 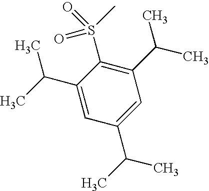 | −17 | 46 | 13 | 35 |
| P10 | 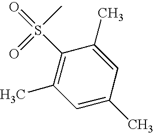 | −14 | 11 | 14 | 35 |
| P11 | 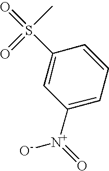 | −23 | 22 | 19 | 27 |
| P12 | 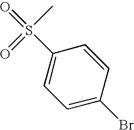 | 54 | 54 | 24 | 29 |
| P13 | 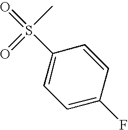 | 75 | 77 | 19 | 31 |
| P14 | 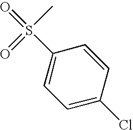 | 60 | 67 | 23 | 25 |
| P15 | 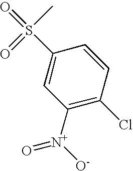 | 50 | 65 | 30 | 34 |
| P16 | 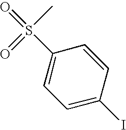 | 71 | 67 | 34 | 35 |
| P17 | 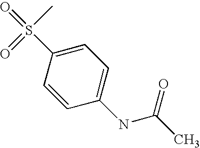 | 77 | 78 | 14 | 36 |
| P18 | 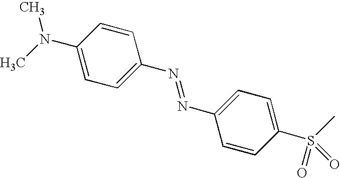 | −20 | 6 | 20 | 36 |
| P19 | 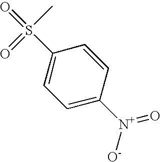 | 63 | 73 | 19 | 30 |
| P20 | 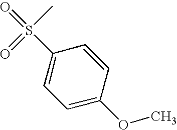 | 78 | 76 | 23 | 43 |
| P21 | 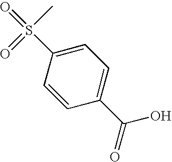 | 23 | 32 | 19 | 29 |
| P22 | 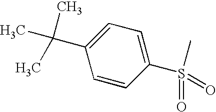 | 29 | 38 | 27 | 31 |
| P23 | 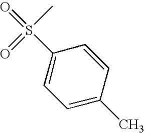 | 64 | 67 | 19 | 32 |
| P24 | 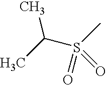 | 5 | 24 | 26 | 36 |
| P25 | 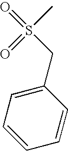 | 62 | 82 | 8 | 33 |
| P26 | 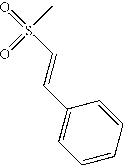 | 37 | 39 | 4 | 23 |
| P27 | 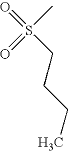 | 16 | 41 | 4 | 23 |
| P28 | 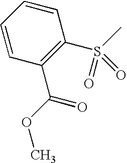 | 55 | 56 | 7 | 28 |
| P29 | 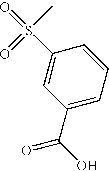 | 35 | 56 | 0 | 21 |
| P30 | 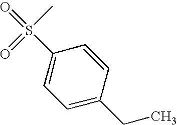 | 53 | 61 | 9 | 17 |
| P31 | 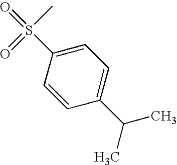 | 40 | 50 | 3 | 16 |
| P32 | 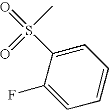 | 58 | 59 | 13 | 28 |
| P33 | 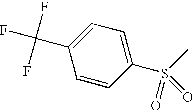 | 56 | 59 | 6 | 32 |
| P34 | 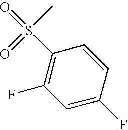 | 60 | 58 | 8 | 23 |
| P35 | 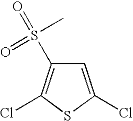 | 37 | 47 | 1 | 22 |
| P36 | 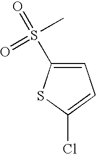 | 54 | 66 | 8 | 26 |
| P37 | 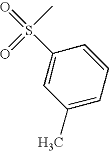 | 58 | 65 | 0 | 27 |
| P38 | 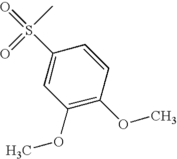 | 73 | 74 | 15 | 35 |
| P39 | 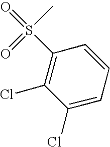 | 24 | 42 | 0 | 25 |
| P40 | 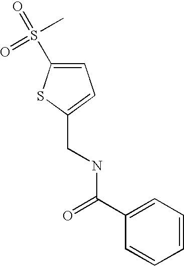 | 61 | 64 | 7 | 33 |
| P41 | 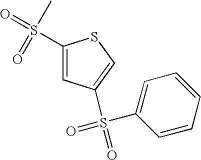 | 80 | 66 | 0 | 26 |
| P42 | 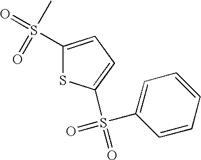 | 55 | 62 | 3 | 19 |
| P43 | 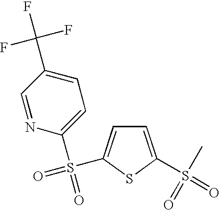 | 70 | 57 | 0 | 17 |
| P44 | 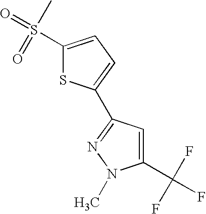 | 55 | 62 | 0 | 25 |
| P45 | 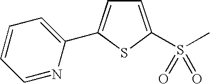 | 65 | 82 | 14 | 27 |
| P46 | 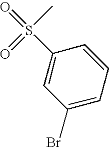 | 59 | 68 | 10 | 20 |
| P47 | 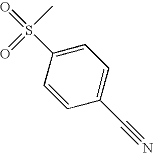 | 81 | 82 | 0 | 26 |
| P48 | 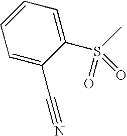 | 59 | 67 | 24 | 31 |
| P49 | 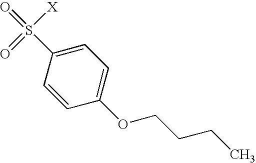 | 36 | 54 | 10 | 32 |
| P50 | 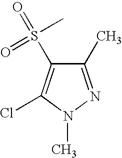 | 30 | 35 | 14 | 25 |
| P51 | 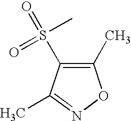 | 3 | 27 | 18 | 21 |
| P52 | 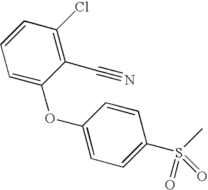 | 49 | 47 | 16 | 22 |
| P53 | 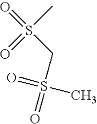 | −23 | 16 | 21 | 27 |
| P54 | 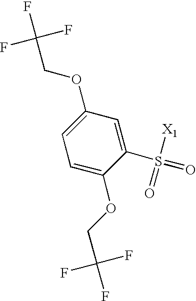 | 17 | 34 | 22 | 23 |
| P55 | 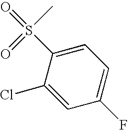 | 43 | 52 | 20 | 25 |
| P56 | 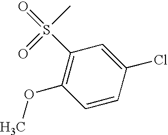 | 21 | 26 | 20 | 34 |
| P57 | 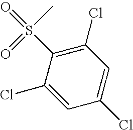 | 23 | 6 | 9 | 31 |
| P58 | 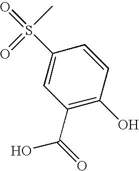 | −16 | 15 | 14 | 30 |
| P59 | 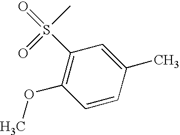 | 17 | 33 | 19 | 24 |
| P60 | 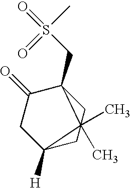 | −1 | 21 | 19 | 25 |
| P61 | 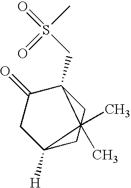 | −34 | 11 | 10 | 28 |
| P62 | 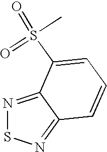 | 74 | 70 | 22 | 26 |
| P63 | 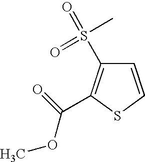 | 71 | 66 | 23 | 40 |
| P64 | 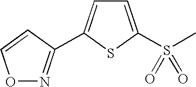 | 80 | 81 | 13 | 31 |
| P65 | 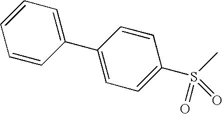 | 48 | 65 | 6 | 31 |
| P66 | 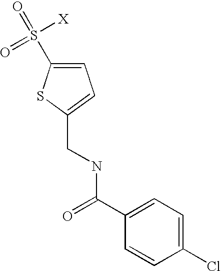 | 55 | 57 | 12 | 34 |
| P67 | 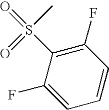 | −8 | 22 | 9 | 25 |
| P68 | 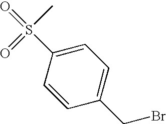 | 72 | 70 | 8 | 25 |
| P69 | 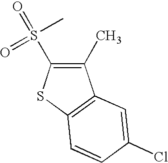 | −2 | 21 | 13 | 30 |
| P70 | 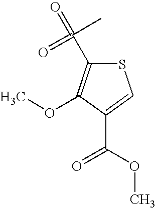 | 37 | 60 | 14 | 27 |
| P71 | 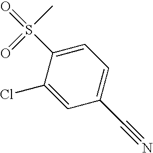 | 57 | 52 | 13 | 21 |
| P72 | 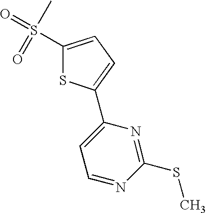 | 61 | 61 | 13 | 35 |
| P73 | 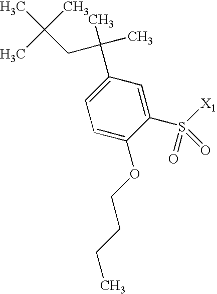 | −28 | 1 | 16 | 30 |
| P74 | 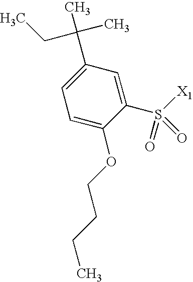 | −30 | 4 | 19 | 27 |
| P75 | 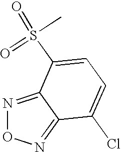 | 60 | 79 | 27 | 43 |
| P76 | 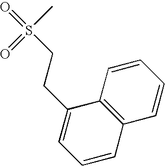 | 9 | 33 | 23 | #N/A |
| P77 | 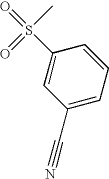 | 19 | 43 | 21 | 20 |
| P78 | 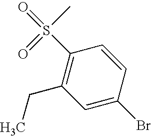 | 17 | 24 | 27 | 23 |
| P79 | 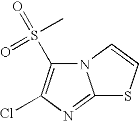 | 53 | 44 | 10 | 18 |
| P80 | 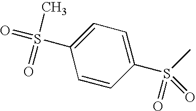 | 81 | 73 | 15 | 29 |
| P81 | 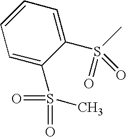 | −5 | 36 | 12 | 19 |
| P82 | 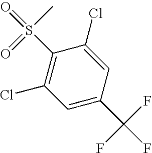 | −23 | 12 | 17 | 24 |
| P83 | 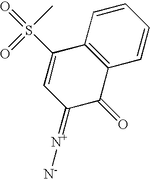 | −11 | 25 | 10 | 26 |
| P84 | 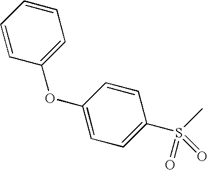 | 28 | 38 | 10 | 26 |
| P85 | 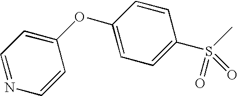 | −28 | 14 | 12 | 26 |
| P86 | 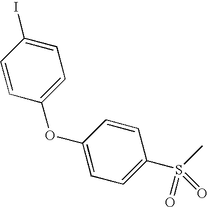 | 38 | 51 | 7 | 26 |
| P87 | 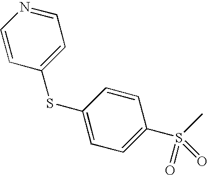 | −25 | −5 | 9 | 22 |
| P88 | 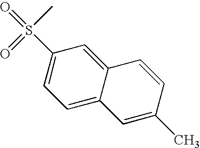 | 44 | 49 | 7 | 34 |

TABLE 4
(I)

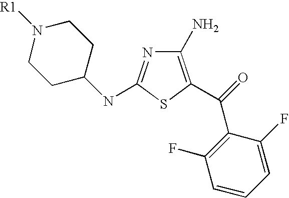

| | | CDK2 | CDK4 | HCT-116 | HCT-116 |
|---|---|---|---|---|---|
| | | % Inhibition | % Inhibition | % Inhibition | % Inhibition |
| Example | R1 | at 0.03 μM | at 0.03 μM | at 0.1 μM | at 0.25 μM |
| Q1 | 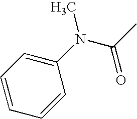 | −7 | 31 | 0 | 38 |
| Q2 | 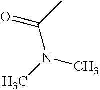 | −43 | 13 | 0.71 | 43 |
| Q3 | 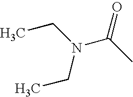 | −43 | 11 | 0 | 42 |
| Q4 | 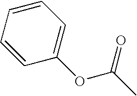 | 59 | 78 | 0 | 41 |
| Q5 | 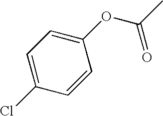 | 45 | 81 | 0.81 | 36 |
| Q6 | 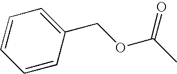 | −32 | 24 | 9 | 38 |
| Q7 | 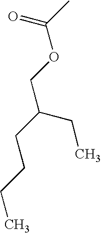 | −42 | 5 | 5 | 39 |
| Q8 | 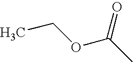 | −13 | 15 | 6 | 45 |
| Q9 | 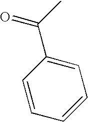 | 13 | 52 | 0 | 36 |
| Q10 | 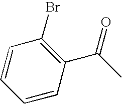 | 23 | 57 | 1 | 42 |
| Q11 | 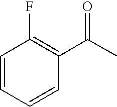 | 30 | 57 | 7 | 43 |
| Q12 | 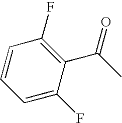 | −20 | 22 | 3 | 46 |
| Q13 | 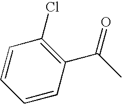 | 13 | 48 | 5 | 43 |
| Q14 | 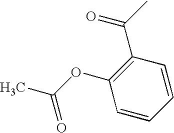 | 59 | 80 | 15 | 45 |
| Q15 | 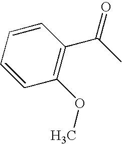 | 25 | 52 | 9 | 45 |
| Q16 | 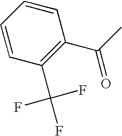 | −12 | 19 | 11 | 50 |
| Q17 | 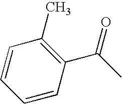 | −11 | 45 | 2 | 34 |
| Q18 | 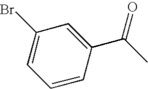 | 44 | 73 | 59 | 92 |
| Q19 | 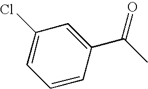 | 21 | 59 | 32 | 83 |
| Q20 | 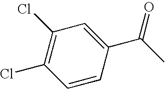 | 33 | 74 | 28 | 69 |
| Q21 | 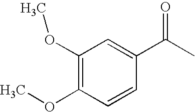 | −23 | 14 | 16 | 51 |
| Q22 | 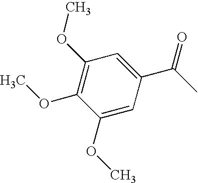 | 24 | 73 | 16 | 48 |
| Q23 | 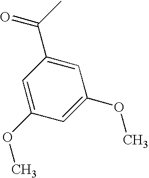 | 20 | 56 | 10 | 42 |
| Q24 | 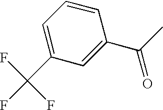 | 31 | 65 | 36 | 71 |
| Q25 | 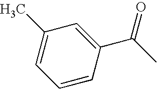 | 30 | 60 | 31 | 85 |
| Q26 | 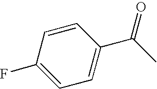 | 18 | 60 | 3 | 42 |
| Q27 | 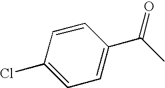 | 32 | 76 | 4 | 40 |
| Q28 | 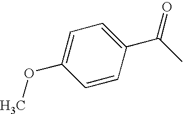 | 53 | 82 | 6 | 41 |
| Q29 | 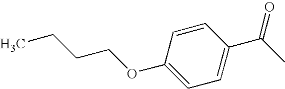 | 21 | 60 | 7 | 50 |
| Q30 | 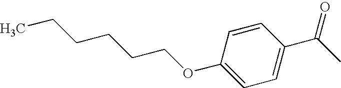 | −4 | 42 | 8 | 47 |
| Q31 | 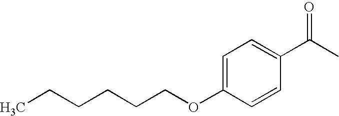 | −2 | 35 | 8 | 41 |
| Q32 | 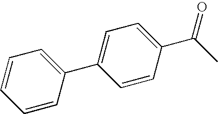 | −11 | 15 | 13 | 54 |
| Q33 | 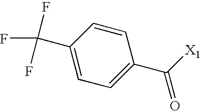 | 23 | 65 | 0 | 16 |
| Q34 | 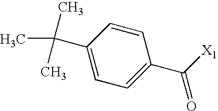 | 28 | 56 | 1 | 27 |
| Q35 | 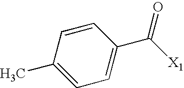 | 35 | 64 | 3 | 21 |
| Q36 | 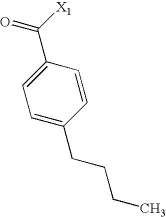 | 16 | 45 | 0.49 | 31 |
| Q37 | 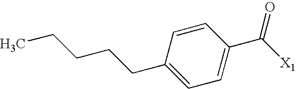 | 12 | 45 | 0 | 31 |
| Q38 | 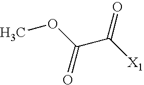 | −12 | 16 | 0 | 21 |
| Q39 | 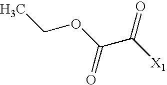 | −13 | 17 | 0 | 25 |
| Q40 | 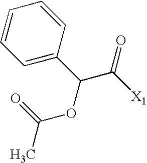 | −5 | 7 | 0 | 20 |
| Q41 | 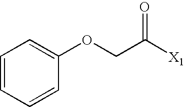 | 24 | 36 | 3 | 15 |
| Q42 | 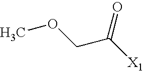 | −4 | 20 | 3 | 30 |
| Q43 | 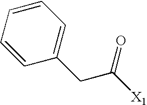 | 16 | 30 | 0 | 24 |
| Q44 | 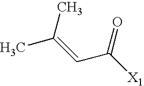 | −19 | 17 | 0 | 30 |
| Q45 | 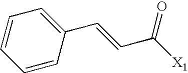 | 21 | 47 | 0 | 31 |
| Q46 | 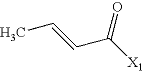 | −9 | 25 | 0 | 27 |
| Q47 | 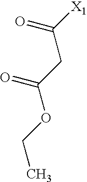 | −13 | 9 | 0 | 25 |
| Q48 | 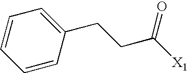 | 5 | 48 | 0 | 19 |
| Q49 | 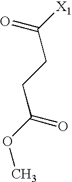 | 16 | 24 | 0 | 28 |
| Q50 | 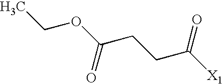 | 24 | 42 | 7 | 39 |
| Q51 | 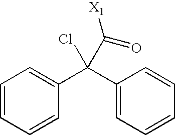 | 4 | 22 | 0 | 34 |
| Q52 | 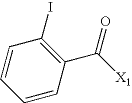 | 24 | 53 | 0 | 24 |
| Q53 | 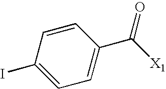 | 60 | 83 | 0 | 22 |
| Q54 | 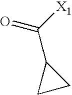 | −2 | 18 | 0 | 35 |
| Q55 | 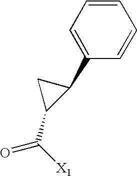 | 29 | 43 | 10 | 31 |
| Q56 | 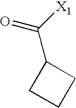 | 12 | 14 | 0 | 35 |
| Q57 | 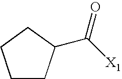 | 32 | 40 | 0 | 32 |
| Q58 | 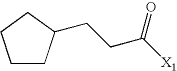 | 29 | 53 | 0 | 25 |
| Q59 | 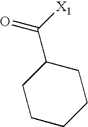 | 10 | 14 | 0 | 30 |
| Q60 | 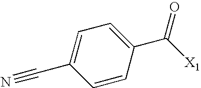 | 37 | 67 | 0 | 37 |
| Q61 | 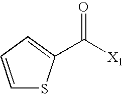 | 41 | 49 | 0 | 34 |
| Q62 | 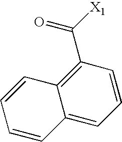 | 33 | 49 | 12 | 53 |
| Q63 | 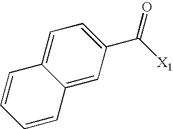 | 39 | 57 | 5 | 25 |
| Q64 | 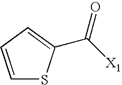 | 45 | 48 | 7 | 25 |
| Q65 | 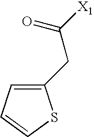 | 39 | 31 | 3 | 27 |
| Q66 | 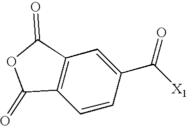 | 7 | 29 | 7 | 34 |
| Q67 | 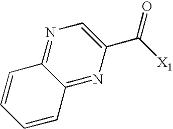 | 13 | 50 | 9 | 30 |
| Q68 | 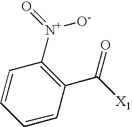 | 41 | 51 | 4 | 33 |
| Q69 | 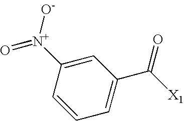 | 54 | 80 | 1 | 24 |
| Q70 | 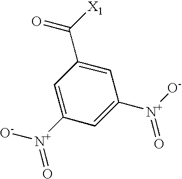 | 19 | 38 | 5 | 26 |
| Q71 | 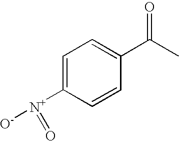 | 28 | 61 | 2 | 30 |
| Q72 | 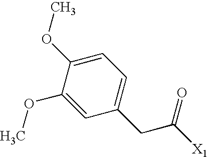 | 18 | 43 | 8 | 29 |
| Q73 | 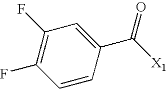 | 52 | 78 | 3 | 33 |
| Q74 | 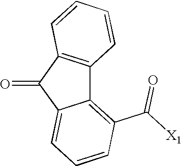 | 2 | 19 | 3 | 35 |
| Q75 | 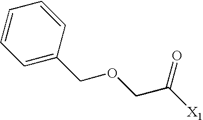 | 9 | 20 | 5 | 39 |
| Q76 | 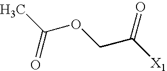 | 27 | 31 | 4 | 36 |
| Q77 | 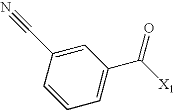 | 44 | 72 | 8 | 33 |
| Q78 | 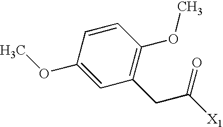 | 39 | 46 | 0.43 | 37 |
| Q79 | 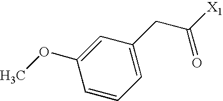 | 51 | 59 | 5 | 33 |
| Q80 | 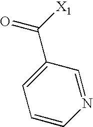 | 20 | 50 | 10 | 27 |
| Q81 | 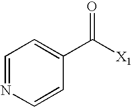 | 6 | 19 | 4 | 37 |
| Q82 | 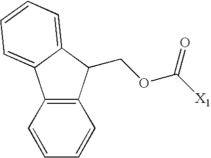 | −12 | 15 | 0 | 28 |
| Q83 | 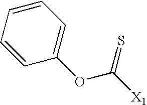 | 72 | 55 | 2 | 25 |
| Q84 | 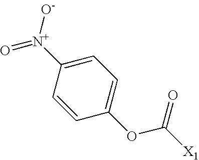 | 63 | 88 | 3 | 40 |
| Q85 | 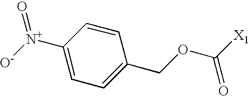 | 42 | 55 | 1 | 31 |
| Q86 | 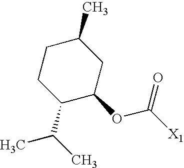 | −45 | 23 | 9 | 29 |
| Q86 | 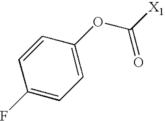 | 47 | 77 | 7 | 36 |
| Q87 | 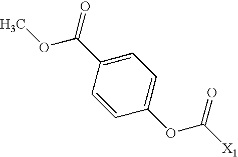 | 54 | 77 | 4 | 37 |

TABLE 5

| | |
|---|---|
| | (I) |
| 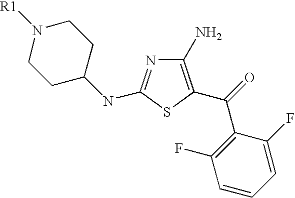 | |

| | | CDK2 | CDK4 | HCT-116 | HCT-116 |
|---|---|---|---|---|---|
| | | % Inhibition | % Inhibition | % Inhibition | 5 Inhibition |
| Example | R¹ | at 0.03 μM | at 0.03 μM | at 0.1 μM | at 0.25 μM |
| R1 | 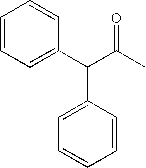 | −34 | 3 | 0 | 32 |
| R2 | 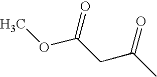 | −24 | 13 | 1 | 49 |
| R3 | 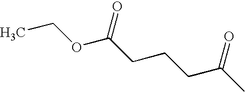 | −6 | 42 | 8 | 37 |
| R4 | 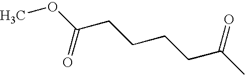 | −5 | 32 | 3 | 47 |
| R5 | 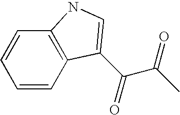 | −31 | 9 | 13 | 49 |
| R6 | 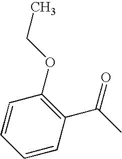 | 14 | 42 | 12 | 51 |
| R7 | 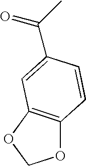 | −3 | 29 | 7 | 46 |
| R8 | 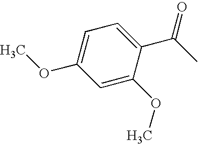 | −11 | 13 | 8 | 41 |
| R9 | 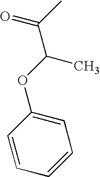 | −15 | 19 | 5 | 37 |
| R10 | 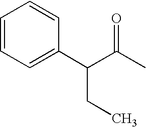 | 5 | 29 | 0 | 40 |
| R11 | 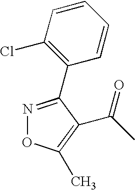 | 4 | 49 | 0 | 37 |
| R12 | 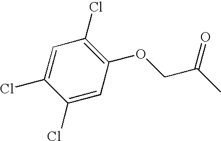 | −10 | 23 | 0 | 48 |
| R13 | 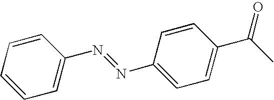 | 32 | 69 | 0 | 42 |
| R14 | 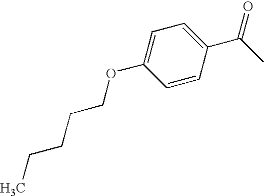 | 19 | 49 | 0 | 46 |
| R15 | 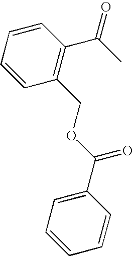 | −9 | 5 | 15 | 45 |
| R16 | 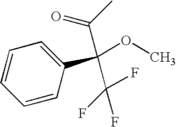 | −29 | 12 | 6 | 41 |
| R17 | 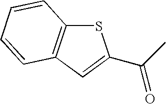 | 66 | 73 | 0 | 45 |
| R18 | 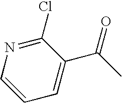 | 25 | 46 | 0 | 45 |
| R19 | 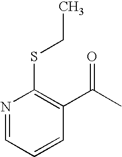 | 37 | 54 | 0 | 46 |
| R20 | 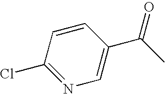 | 59 | 85 | 0 | 42 |
| R21 | 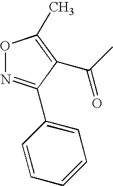 | 2 | 38 | 0 | 47 |
| R22 | 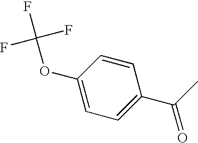 | 22 | 58 | 7 | 48 |
| R23 | 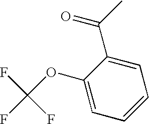 | −6 | 34 | 2 | 38 |
| R24 | 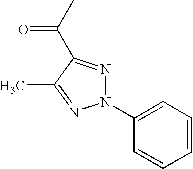 | 20 | 49 | 5 | 39 |
| R25 | 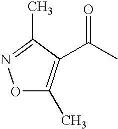 | −9 | 22 | 0 | 43 |
| R26 | 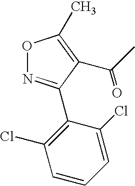 | 17 | 64 | 0 | 46 |
| R27 | 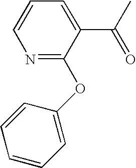 | 6 | 19 | 0 | 43 |
| R28 | 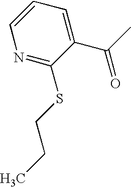 | 58 | 66 | 0 | 41 |
| R29 | 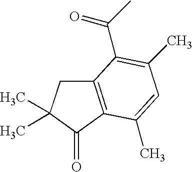 | 2 | 23 | 0 | 36 |
| R30 | 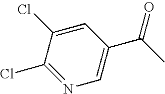 | 31 | 63 | 13 | 43 |
| R31 | 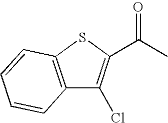 | 57 | 60 | 0 | 42 |
| R32 | 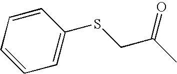 | 38 | 65 | 0 | 48 |
| R33 | 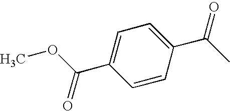 | 58 | 80 | 1 | 49 |
| R34 | 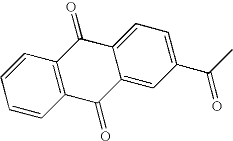 | 35 | 60 | 0 | 55 |
| R35 | 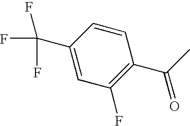 | 19 | 21 | 0 | 49 |
| R36 | 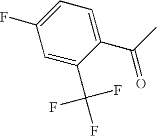 | 21 | 17 | 0 | 51 |
| R37 | 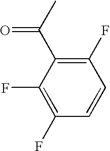 | 27 | 22 | 0 | 48 |
| R38 | 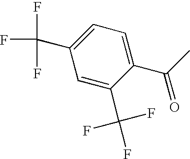 | 0 | 35 | 0 | 33 |
| R39 | 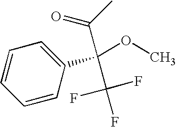 | −6 | 16 | 5 | 51 |
| R40 | 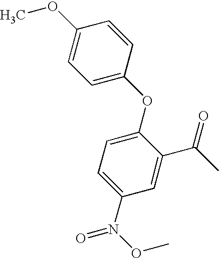 | 41 | 66 | 0 | 43 |
| R41 | 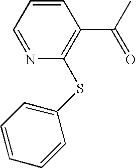 | 24 | 32 | 0 | 41 |
| R42 | 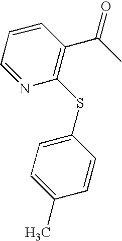 | 49 | 53 | 0 | 48 |
| R43 | 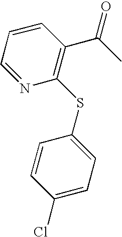 | −73 | 2 | 4 | 46 |
| R44 | 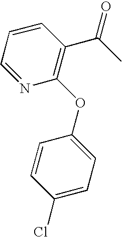 | 16 | 25 | 0 | 41 |
| R45 | 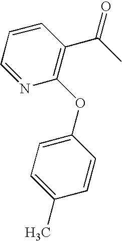 | 37 | 49 | 2 | 36 |
| R46 | 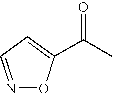 | 71 | 83 | 0.47 | 37 |
| R47 | 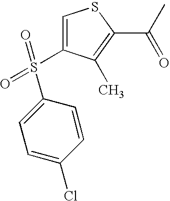 | −7 | 20 | 8 | 45 |
| R48 | 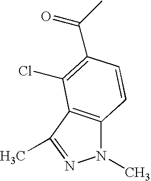 | 16 | 32 | 0 | 50 |
| R49 | 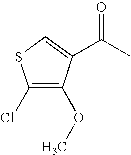 | 34 | 55 | 0 | 61 |
| R50 | 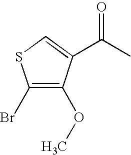 | 51 | 44 | 0 | 48 |
| R51 | 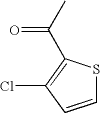 | 62 | 48 | 0 | 37 |
| R52 | 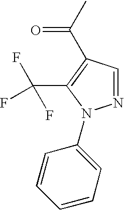 | 5 | 23 | 2 | 49 |
| R53 | 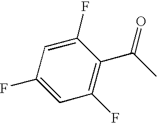 | 24 | 32 | 0 | 30 |
| R54 | 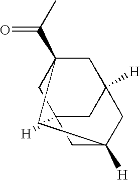 | 21 | 38 | 0 | 39 |
| R55 | 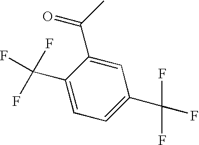 | 11 | 37 | 5 | 51 |
| R56 | 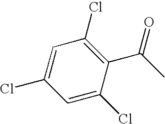 | 14 | 8 | 0 | 43 |
| R57 | 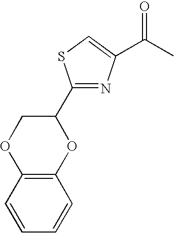 | 23 | 36 | 0 | 47 |
| R58 | 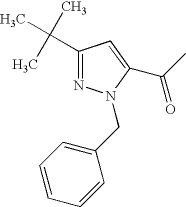 | 41 | 72 | 5 | 44 |
| R59 | 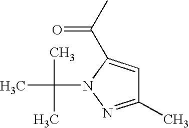 | 16 | 25 | 4 | 47 |
| R60 | 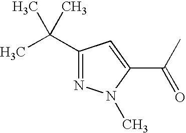 | 45 | 69 | 0 | 29 |
| R61 | 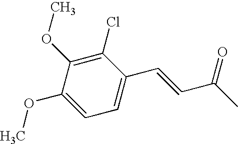 | 63 | 59 | 0 | 37 |
| R62 | 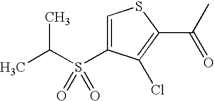 | 65 | 78 | 0 | 38 |
| R63 | 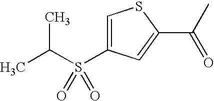 | 11 | 12 | 0 | 38 |
| R64 | 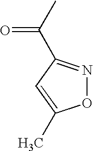 | 9 | 10 | 0 | 42 |
| R65 | 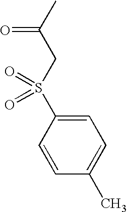 | 9 | 24 | 3 | 31 |
| R66 | 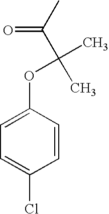 | 16 | 27 | 0.54 | 42 |
| R67 | 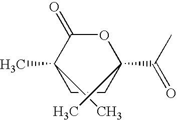 | 11 | 22 | 4 | 40 |
| R68 | 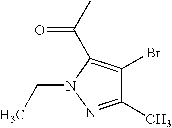 | 22 | 15 | 0 | 33 |
| R69 | 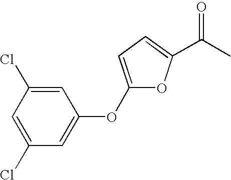 | 29 | 35 | 0 | 41 |
| R70 | 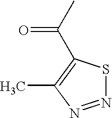 | 21 | 12 | 0 | 44 |
| R71 | 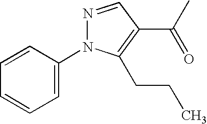 | 33 | 51 | 0 | 44 |
| R72 | 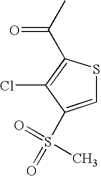 | 57 | 59 | 6 | 43 |
| R73 | 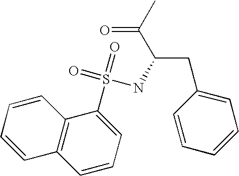 | 6 | 19 | 4 | 38 |
| R74 | 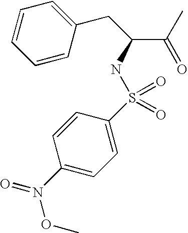 | 3 | 20 | 0.12 | 42 |
| R75 | 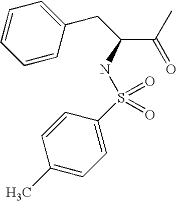 | 26 | 13 | 0 | 41 |
| R76 | 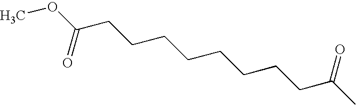 | 53 | 64 | 0 | 38 |
| R77 | 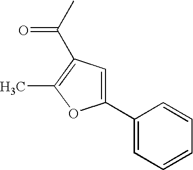 | 19 | 15 | 0 | 44 |
| R78 | 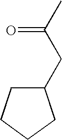 | 20 | 19 | 0 | 47 |
| R79 | 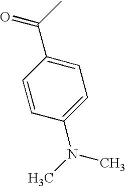 | 14 | 16 | 0 | 47 |
| R80 | 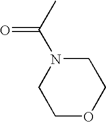 | 7 | 37 | 0 | 47 |
| R81 | 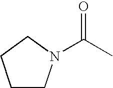 | −10 | 29 | 13 | 43 |
| R82 | 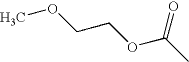 | −11 | 17 | 0 | 50 |
| R83 | 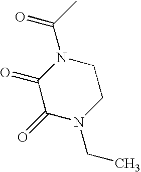 | 21 | 52 | 0 | 45 |
| R84 | 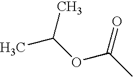 | 4 | 21 | 0 | 41 |
| R85 | 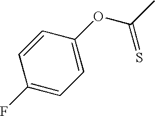 | 90 | 81 | 0 | 47 |
| R86 | 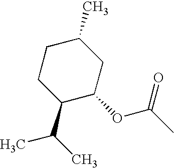 | 9 | 9 | 0 | 34 |
| R87 | 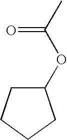 | 17 | 28 | 0 | 36 |
| R88 | 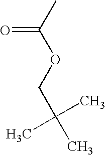 | 6 | −3 | 0 | 42 |
| R89 | 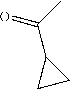 | 3 | 5 | 0 | 46 |
| R90 | 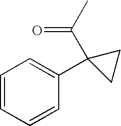 | −2 | −6 | 0 | 19 |
| R91 | 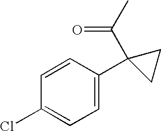 | −1 | 14 | 2 | 41 |
| R92 | 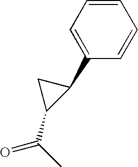 | −9 | −2 | 0 | 40 |
| R93 | 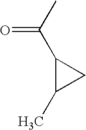 | −10 | −1 | 0 | 42 |
| R94 | 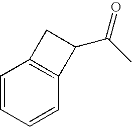 | 10 | −5 | 0 | 42 |
| R95 | 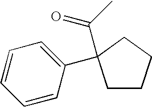 | −13 | −27 | 0 | 38 |
| R96 | 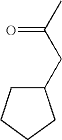 | −20 | −18 | 2 | 36 |
| R97 | 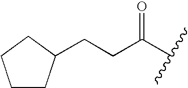 | 13 | 17 | 0 | 39 |
| R98 | 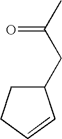 | 8 | 11 | 0 | 44 |
| R99 | 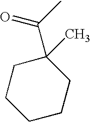 | −5 | −4 | 5 | 49 |
| R100 | 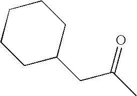 | −5 | 10 | 0 | 49 |
| R101 | 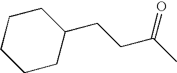 | 15 | 39 | 7 | 45 |
| R102 | 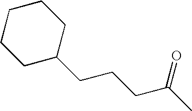 | 18 | 34 | 0 | 43 |
| R103 | 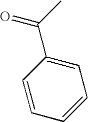 | 4 | 18 | 0 | 45 |
| R104 | 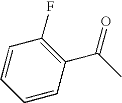 | 5 | −8 | 0 | 38 |
| R105 | 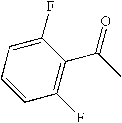 | 8 | 9 | 0 | 44 |
| R106 | 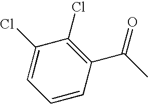 | 2 | 19 | 2 | 72 |
| R107 | 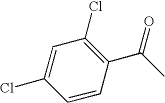 | 44 | 63 | 8 | 48 |
| R108 | 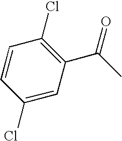 | 56 | 72 | 5 | 58 |
| R109 | 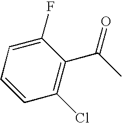 | 21 | 34 | 14 | 47 |
| R110 | 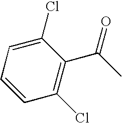 | 24 | 30 | 3 | 48 |
| R111 | 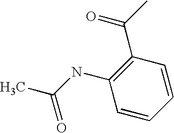 | 11 | 25 | 4 | 52 |
| R112 | 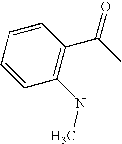 | 12 | 21 | 39 | 93 |
| R113 | 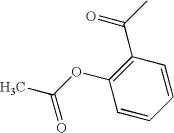 | 44 | 48 | 0 | 40 |
| R114 | 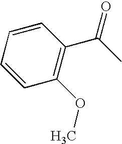 | 60 | 65 | 0 | 42 |
| R115 | 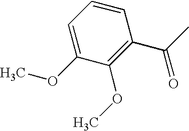 | 42 | 58 | 0 | 49 |
| R116 | 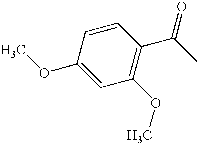 | 49 | 67 | 9 | 44 |
| R117 | 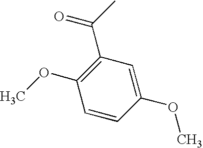 | 53 | 66 | 7 | 45 |
| R118 | 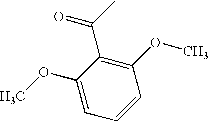 | 36 | 33 | 7 | 47 |
| R119 | 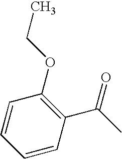 | 27 | 31 | 3 | 53 |
| R120 | 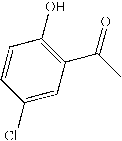 | 18 | 4 | 0 | 47 |
| R121 | 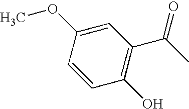 | 57 | 60 | 0 | 37 |
| R122 | 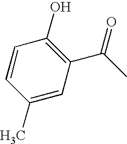 | 61 | 67 | 0 | 48 |
| R123 | 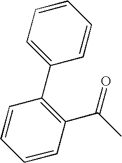 | 5 | 39 | 0 | 38 |
| R124 | 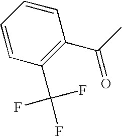 | 18 | 38 | 0 | 35 |
| R125 | 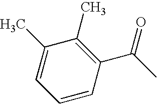 | 18 | 33 | 0 | 42 |
| R126 | 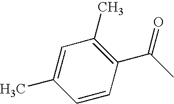 | 43 | 57 | 0 | 33 |
| R127 | 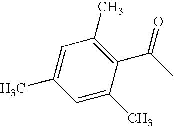 | 28 | 20 | 0 | 36 |
| R128 | 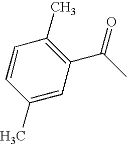 | 14 | 7 | 0 | 56 |
| R129 | 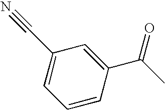 | 41 | 62 | 0 | 33 |
| R130 | 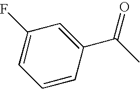 | 59 | 77 | 0 | 20 |
| R131 | 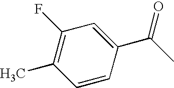 | 42 | 58 | 1 | 44 |
| R132 | 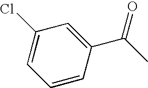 | 28 | 55 | 15 | 60 |
| R133 | 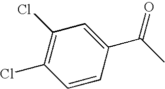 | 21 | 52 | 5 | 56 |
| R134 | 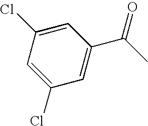 | 29 | 51 | 45 | 95 |
| R135 | 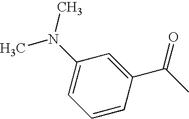 | 27 | 50 | 0 | 38 |
| R136 | 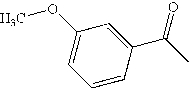 | 26 | 45 | 0 | 37 |
| R137 | 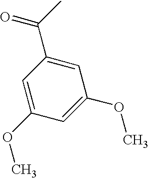 | 55 | 73 | 0 | 38 |
| R138 | 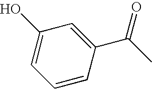 | 67 | 70 | 0 | 40 |
| R139 | 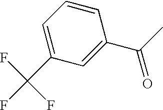 | 50 | 75 | 20 | 62 |
| R140 | 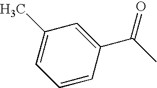 | 38 | 69 | 32 | 78 |
| R141 | 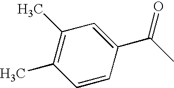 | 68 | 82 | 33 | 77 |
| R142 | 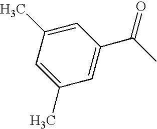 | 67 | 83 | 86 | 99 |
| R143 | 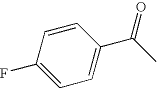 | 30 | 59 | 0 | 40 |
| R144 | 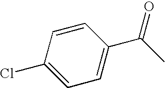 | 20 | 46 | 0 | 38 |
| R145 | 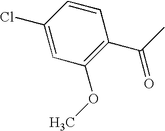 | 45 | 53 | 0 | 38 |
| R146 | 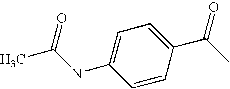 | 74 | 83 | 0 | 10 |
| R147 | 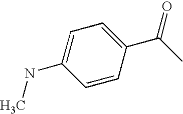 | 63 | 78 | 0 | 41 |
| R148 | 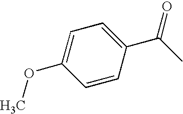 | 20 | 49 | 0 | 42 |
| R149 | 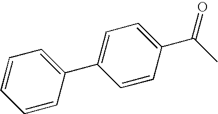 | 28 | 51 | 11 | 43 |
| R150 | 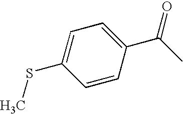 | 40 | 62 | 2 | 39 |
| R151 | 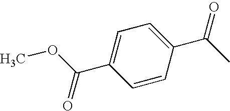 | 24 | 39 | 0 | 43 |
| R152 | 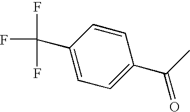 | 17 | 41 | 0 | 40 |
| R153 | 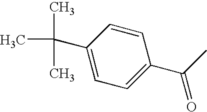 | 37 | 58 | 0 | 44 |
| R154 | 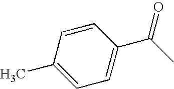 | 50 | 63 | 0 | 27 |
| R155 | 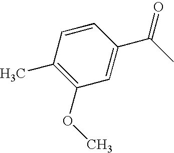 | 45 | 67 | 0 | 67 |
| R156 | 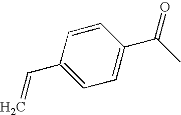 | 65 | 89 | 0 | 43 |
| R157 | 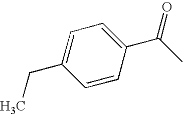 | 22 | 47 | 0 | 42 |
| R158 | 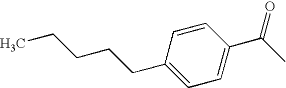 | 24 | 44 | 0 | 41 |
| R159 | 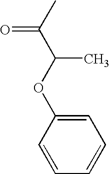 | 14 | 36 | 0 | 46 |
| R160 | 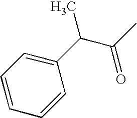 | 7 | 6 | 0 | 37 |
| R161 | 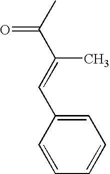 | 26 | 53 | 0 | 34 |
| R162 | 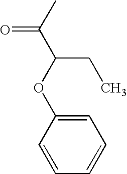 | 27 | 32 | 0 | 34 |
| R163 | 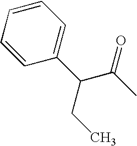 | 26 | 28 | 0 | 44 |
| R164 | 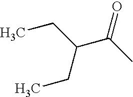 | 9 | 19 | 0 | 41 |
| R165 | 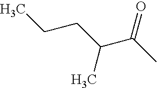 | 3 | 22 | 0 | 42 |
| R166 | 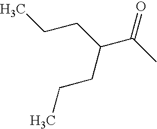 | 17 | 37 | 0 | 44 |
| R167 | 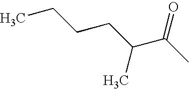 | −2 | 11 | 0 | 42 |
| R168 | 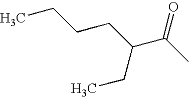 | 6 | −4 | 0 | 42 |
| R169 | 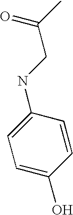 | 22 | 40 | 0 | 39 |
| R170 | 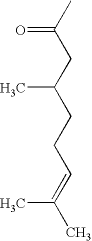 | 21 | 22 | 0 | 23 |
| R171 | 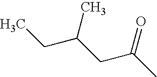 | 23 | 39 | 0 | 46 |
| R172 | 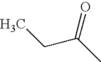 | 34 | 49 | 0 | 49 |
| R173 | 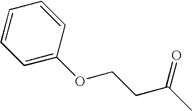 | 23 | 43 | 0 | 43 |
| R174 | 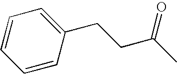 | 21 | 42 | 0 | 48 |
| R175 | 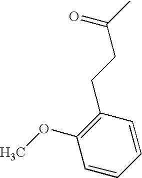 | 19 | 41 | 0 | 47 |
| R176 | 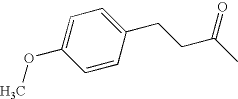 | 2 | 4 | 1 | 43 |

TABLE 6

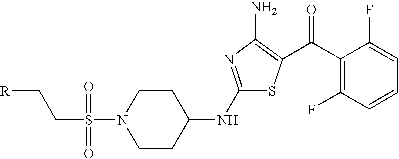

| | | | | % Inhibition |
|---|---|---|---|---|
| | | | | HCT116 |
| Example | PLATE | WELL | R | @ 250 nM |
| S1 | CC34-1 | A1 | 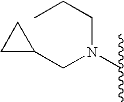 | 36 |
| S2 | CC34-1 | A2 | 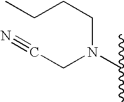 | 18 |
| S3 | CC34-1 | A3 | 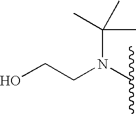 | 25 |
| S4 | CC34-1 | A4 | 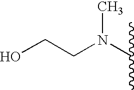 | 25 |
| S5 | CC34-1 | A5 | 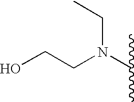 | 26 |
| S6 | CC34-1 | A6 | 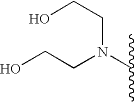 | 13 |
| S7 | CC34-1 | A7 | 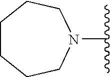 | 22 |
| S8 | CC34-1 | A8 | 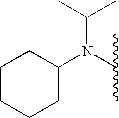 | 91 |
| S9 | CC34-1 | A9 | 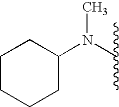 | 43 |
| S10 | CC34-1 | A10 | 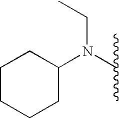 | 62 |
| S11 | CC34-1 | A11 | 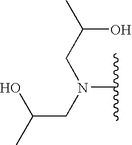 | 13 |
| S12 | CC34-1 | B1 | 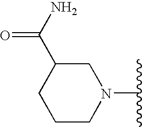 | 19 |
| S13 | CC34-1 | B2 | 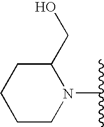 | 24 |
| S14 | CC34-1 | B3 | 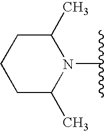 | 91 |
| S15 | CC34-1 | B4 | 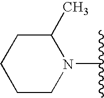 | 46 |
| S16 | CC34-1 | B5 | 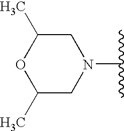 | 20 |
| S17 | CC34-1 | B6 | 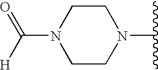 | 19 |
| S18 | CC34-1 | B7 | 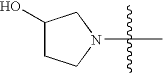 | 14 |
| S19 | CC34-1 | B8 | 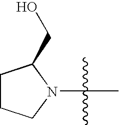 | 15 |
| S20 | CC34-1 | B9 | 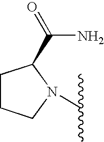 | 18 |
| S21 | CC34-1 | B10 | 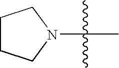 | 35 |
| S22 | CC34-1 | B11 | 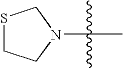 | 18 |
| S23 | CC34-1 | C1 | 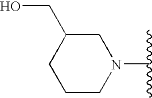 | 18 |
| S24 | CC34-1 | C2 | 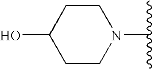 | 14 |
| S25 | CC34-1 | C3 | 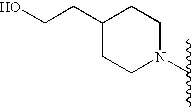 | 10 |
| S26 | CC34-1 | C4 | 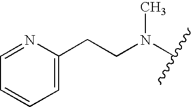 | 17 |
| S27 | CC34-1 | C5 | 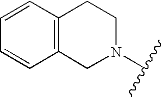 | 27 |
| S28 | CC34-1 | C6 | 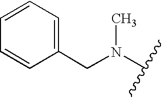 | 80 |
| S29 | CC34-1 | C7 | 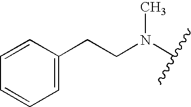 | 18 |
| S30 | CC34-1 | C8 | 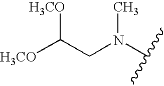 | 18 |
| S31 | CC34-1 | C9 | 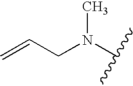 | 27 |
| S32 | CC34-1 | C10 | 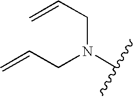 | 69 |
| S33 | CC34-1 | C11 | 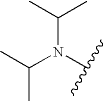 | 79 |
| S34 | CC34-1 | D1 | 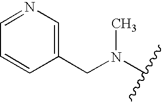 | 21 |
| S35 | CC34-1 | D2 | 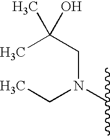 | 58 |
| S36 | CC34-1 | D3 | 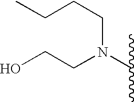 | 17 |
| S37 | CC34-1 | D4 | 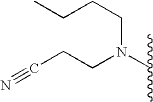 | 24 |
| S38 | CC34-1 | D5 | 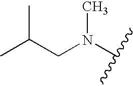 | 55 |
| S39 | CC34-1 | D6 | 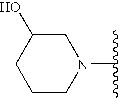 | 14 |
| S40 | CC34-1 | D7 | 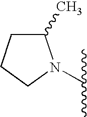 | 93 |
| S41 | CC34-1 | D8 | 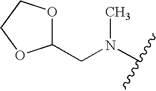 | 18 |
| S42 | CC34-1 | D9 | 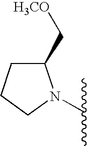 | 12 |
| S43 | CC34-1 | D10 | 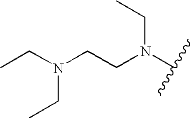 | 13 |
| S44 | CC34-1 | D11 | 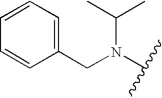 | 55 |
| S45 | CC34-1 | E1 | 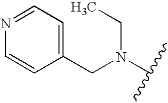 | 13 |
| S46 | CC34-1 | E2 | 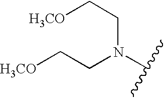 | 58 |
| S47 | CC34-1 | E3 | 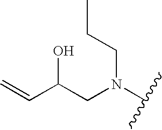 | 8 |
| S48 | CC34-1 | E4 | 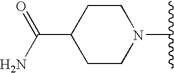 | 17 |
| S49 | CC34-1 | E5 | 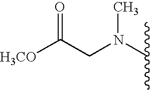 | 33 |
| S50 | CC34-1 | E6 | 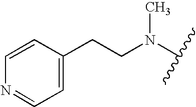 | 17 |
| S51 | CC34-1 | E7 | 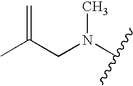 | 83 |
| S52 | CC34-1 | E8 | 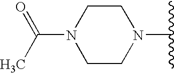 | 18 |
| S53 | CC34-1 | E9 | 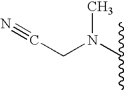 | 17 |
| S54 | CC34-1 | E10 | 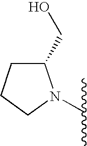 | 16 |
| S55 | CC34-1 | E11 | 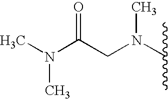 | 7 |
| S56 | CC34-1 | F1 | 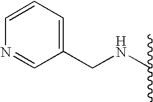 | 73 |
| S57 | CC34-1 | F2 | 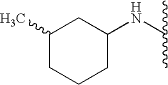 | 73 |
| S58 | CC34-1 | F3 | 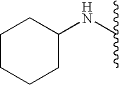 | 50 |
| S59 | CC34-1 | F4 | 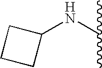 | 40 |
| S60 | CC34-1 | F5 | 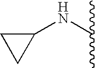 | 72 |
| S61 | CC34-1 | F6 | 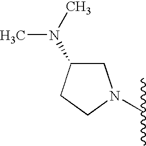 | 8 |
| S62 | CC34-1 | F7 | 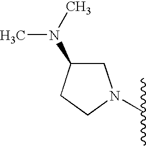 | 6 |
| S63 | CC34-1 | F8 | 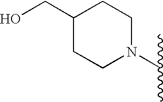 | 4 |
| S64 | CC34-1 | F9 | 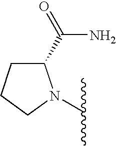 | 12 |
| S65 | CC34-1 | F10 | 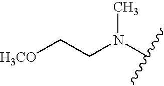 | 8 |
| S66 | CC34-1 | F11 | 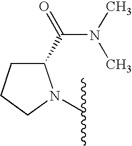 | 14 |
| S67 | CC34-1 | G1 | 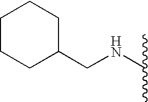 | 40 |
| S68 | CC34-1 | G2 | 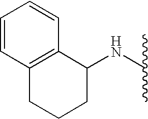 | 21 |
| S69 | CC34-1 | G3 | 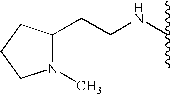 | 31 |
| S70 | CC34-1 | G4 | 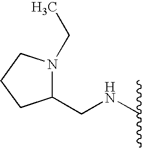 | 23 |
| S71 | CC34-1 | G5 | 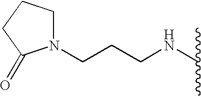 | 23 |
| S72 | CC34-1 | G6 | 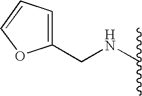 | 19 |
| S73 | CC34-1 | G7 | 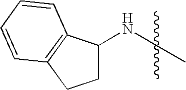 | 16 |
| S74 | CC34-1 | G8 | 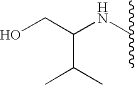 | 9 |
| S75 | CC34-1 | G9 | 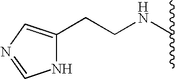 | 12 |
| S76 | CC34-1 | G10 | 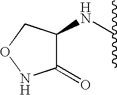 | 11 |
| S77 | CC34-1 | G11 | 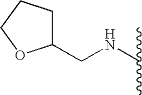 | 9 |
| S78 | CC34-1 | H1 | 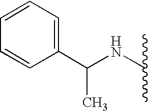 | 49 |
| S79 | CC34-1 | H2 | 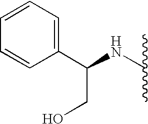 | 14 |
| S80 | CC34-1 | H3 | 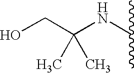 | 13 |
| S81 | CC34-1 | H4 | 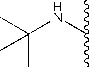 | 82 |
| S82 | CC34-1 | H5 | 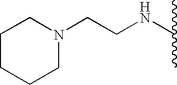 | 15 |
| S83 | CC34-1 | H6 | 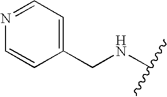 | 6 |
| S84 | CC34-1 | H7 | 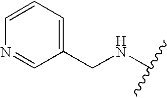 | 12 |
| S85 | CC34-1 | H8 | 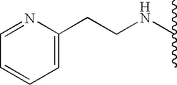 | 19 |
| S86 | CC34-1 | H9 | 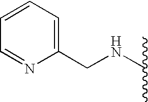 | 11 |
| S87 | CC34-1 | H10 | 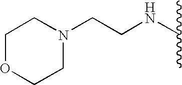 | 13 |
| S88 | CC34-1 | H11 | 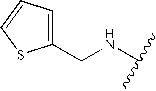 | 28 |
| S89 | CC34-2 | A1 | 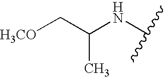 | 17 |
| S90 | CC34-2 | A2 | 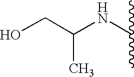 | 7 |
| S91 | CC34-2 | A3 | 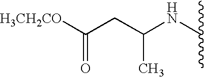 | 10 |
| S92 | CC34-2 | A4 | 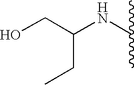 | 8 |
| S93 | CC34-2 | A5 | 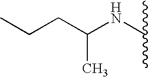 | 33 |
| S94 | CC34-2 | A6 | 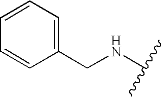 | 64 |
| S95 | CC34-2 | A7 | 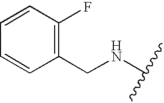 | 51 |
| S96 | CC34-2 | A8 | 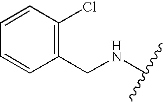 | 48 |
| S97 | CC34-2 | A9 | 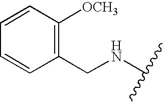 | 25 |
| S98 | CC34-2 | A10 | 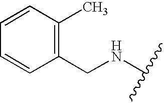 | 49 |
| S99 | CC34-2 | A11 | 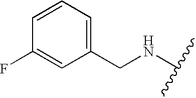 | 43 |
| S100 | CC34-2 | B1 | 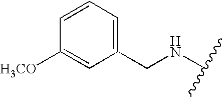 | 38 |
| S101 | CC34-2 | B2 | 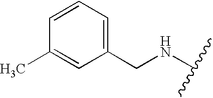 | 80 |
| S102 | CC34-2 | B3 | 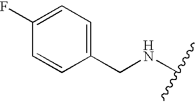 | 57 |
| S103 | CC34-2 | B4 | 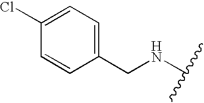 | 25 |
| S104 | CC34-2 | B5 | 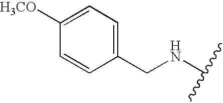 | 31 |
| S105 | CC34-2 | B6 | 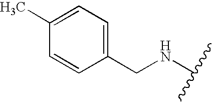 | 33 |
| S106 | CC34-2 | B7 | 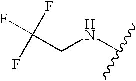 | 98 |
| S107 | CC34-2 | B8 | 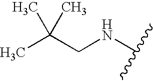 | 98 |
| S108 | CC34-2 | B9 | 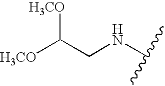 | 16 |
| S109 | CC34-2 | B10 | 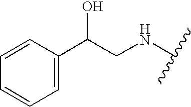 | 19 |
| S110 | CC34-2 | B11 | 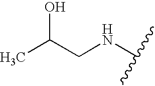 | 16 |
| S111 | CC34-2 | C1 | 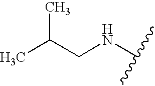 | 54 |
| S112 | CC34-2 | C2 | 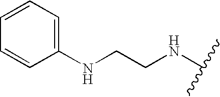 | 15 |
| S113 | CC34-2 | C3 | 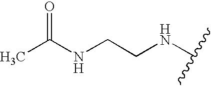 | 51 |
| S114 | CC34-2 | C4 | 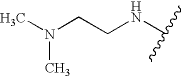 | 49 |
| S115 | CC34-2 | C5 | 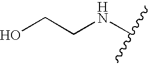 | 68 |
| S116 | CC34-2 | C6 | 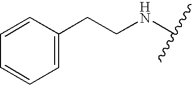 | 60 |
| S117 | CC34-2 | C7 | 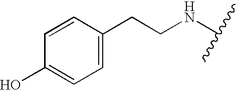 | 86 |
| S118 | CC34-2 | C8 | 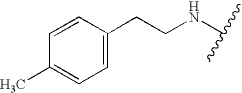 | 25 |
| S119 | CC34-2 | C9 | 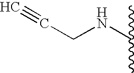 | 20 |
| S120 | CC34-2 | C10 | 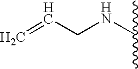 | 17 |
| S121 | CC34-2 | C11 | 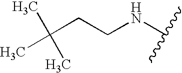 | 20 |
| S122 | CC34-2 | D1 | 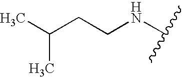 | 54 |
| S123 | CC34-2 | D2 | 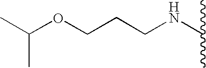 | 15 |
| S124 | CC34-2 | D3 | 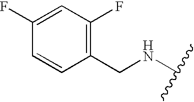 | 51 |
| S125 | CC34-2 | D4 | 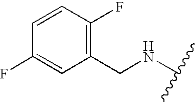 | 49 |
| S126 | CC34-2 | D5 | 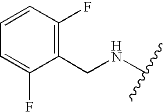 | 68 |
| S127 | CC34-2 | D6 | 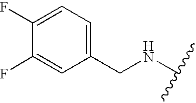 | 60 |
| S128 | CC34-2 | D7 | 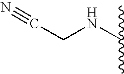 | 86 |
| S129 | CC34-2 | D8 | 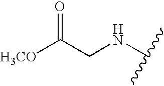 | 25 |
| S130 | CC34-2 | D9 | 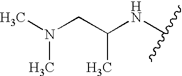 | 20 |
| S131 | CC34-2 | D10 | 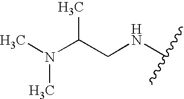 | 17 |
| S132 | CC34-2 | D11 | 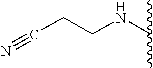 | 20 |
| S133 | CC34-2 | E1 | 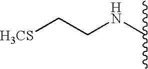 | 18 |
| S134 | CC34-2 | E2 | 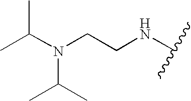 | 17 |
| S135 | CC34-2 | E3 | 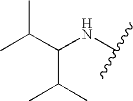 | 100 |
| S136 | CC34-2 | E4 | 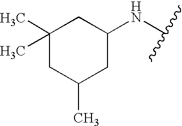 | 81 |
| S137 | CC34-2 | E5 | 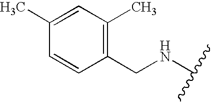 | 43 |
| S138 | CC34-2 | E6 | 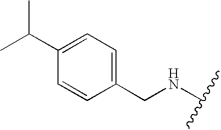 | 22 |
| S139 | CC34-2 | E7 | 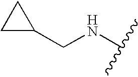 | 39 |
| S140 | CC34-2 | E8 | 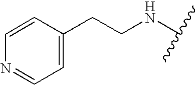 | 25 |
| S141 | CC34-2 | E9 | 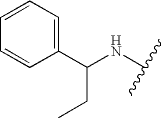 | 78 |
| S142 | CC34-2 | E10 | 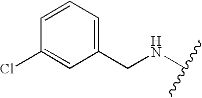 | 80 |
| S143 | CC34-2 | E11 | 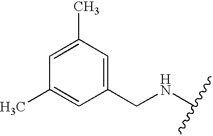 | 55 |
| S144 | CC34-2 | F1 | 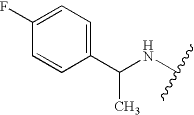 | 43 |
| S145 | CC34-2 | F2 | 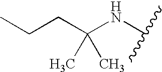 | 96 |
| S146 | CC34-2 | F3 | 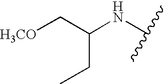 | 42 |
| S147 | CC34-2 | F4 | 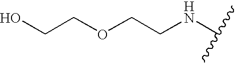 | 23 |
| S148 | CC34-2 | F5 | 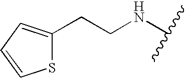 | 34 |
| S149 | CC34-2 | F6 | 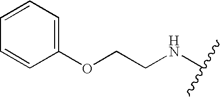 | 36 |
| S150 | CC34-2 | F7 | 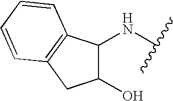 | 33 |
| S151 | CC34-2 | F8 | 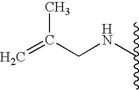 | 50 |
| S152 | CC34-2 | F9 | 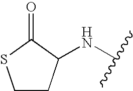 | 95 |
| S153 | CC34-2 | F10 | 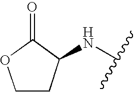 | 100 |
| S154 | CC34-2 | F11 | 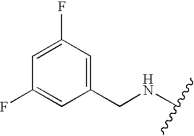 | 31 |
| S155 | CC34-2 | G1 | 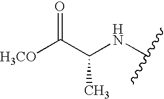 | 96 |
| S156 | CC34-2 | G2 | 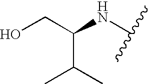 | 17 |
| S157 | CC34-2 | G3 | 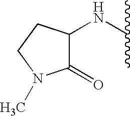 | 16 |
| S158 | CC34-2 | G4 | 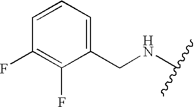 | 73 |
| S159 | CC34-2 | G5 | 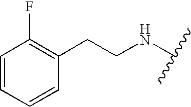 | 44 |
| S160 | CC34-2 | G6 | 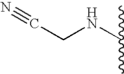 | 30 |
| S161 | CC34-2 | G7 | 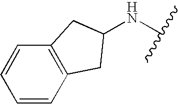 | 45 |
| S162 | CC34-2 | G8 | 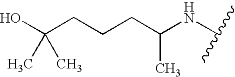 | 22 |
| S163 | CC34-2 | G9 | 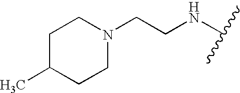 | 25 |
| S164 | CC34-2 | G10 | 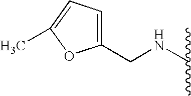 | 28 |
| S165 | CC34-2 | G11 | 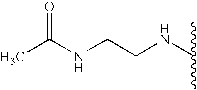 | 25 |
| S166 | CC34-2 | H1 | 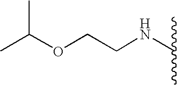 | 9 |
| S167 | CC34-2 | H2 | 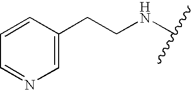 | 3 |
| S168 | CC34-2 | H3 | 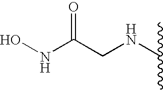 | 16 |
| S169 | CC34-2 | H4 | 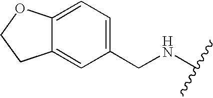 | 17 |
| S170 | CC34-2 | H5 | 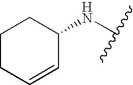 | 15 |
| S171 | CC34-2 | H6 | 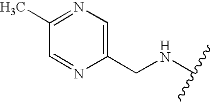 | 18 |
| S172 | CC34-2 | H7 | 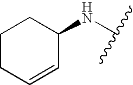 | 20 |
| S173 | CC34-2 | H8 | 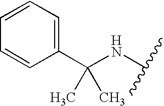 | 35 |
| S174 | CC34-2 | H9 | 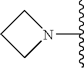 | 10 |
| S175 | CC34-2 | H10 | 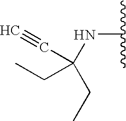 | 99 |
| S176 | CC34-2 | H11 | 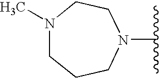 | 17 |

TABLE 7

| | | % inhibition of CDK2 | % inhibition HCT-116 |
|---|---|---|---|
| Example | Structure | (@ 0.05 μM) | in MTT @ 0.175 uM |
| V1 | 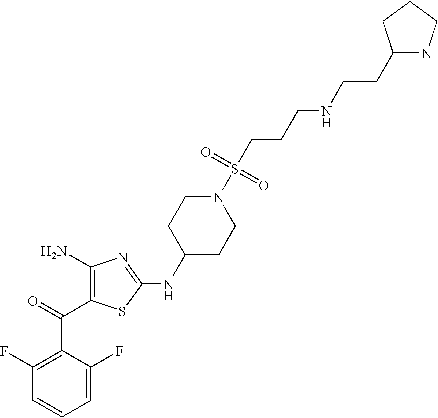 | 80 | 7 |
| V2 | 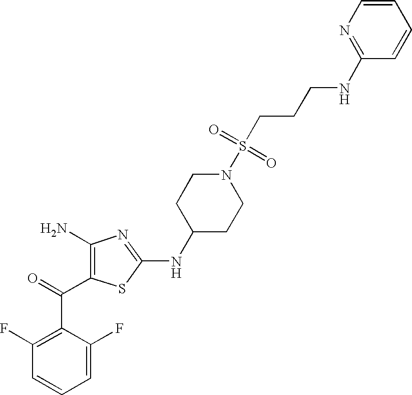 | 85 | 6 |
| V3 | 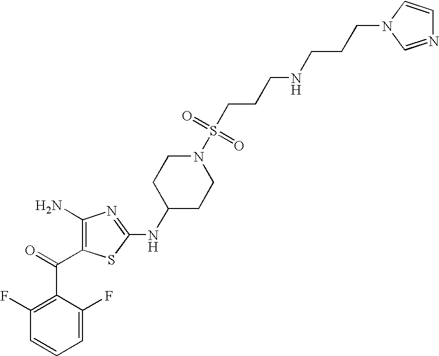 | 84 | 17 |
| V4 | 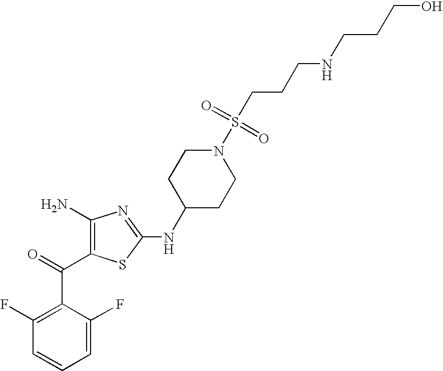 | 75 | 19 |
| V5 | 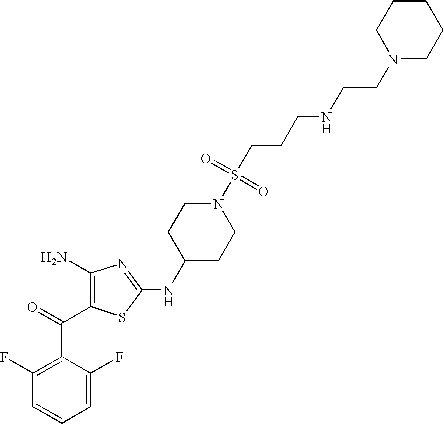 | 79 | 7 |
| V6 | 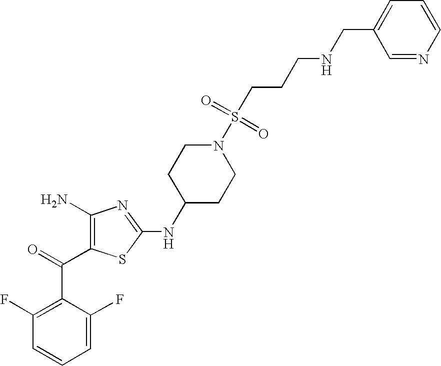 | 80 | 1 |
| V7 | 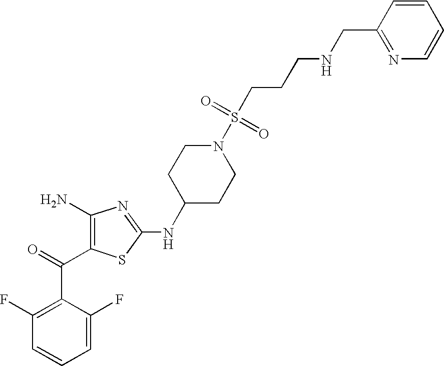 | 82 | 0 |
| V8 | 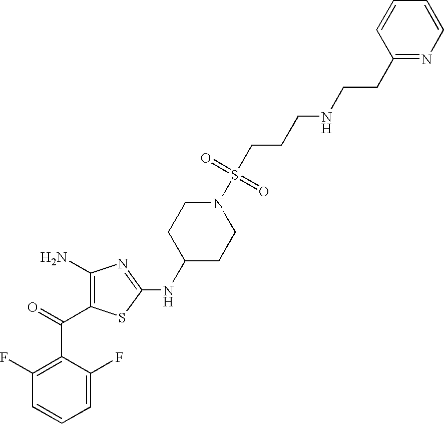 | 81 | 18 |
| V9 | 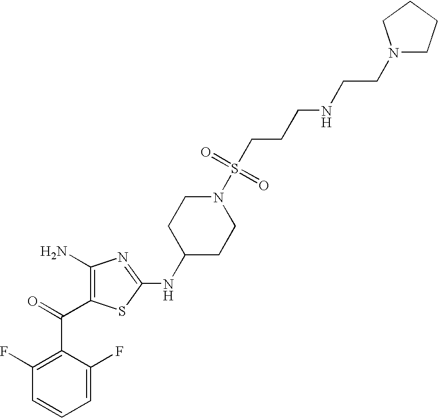 | 78 | 13 |
| V10 | 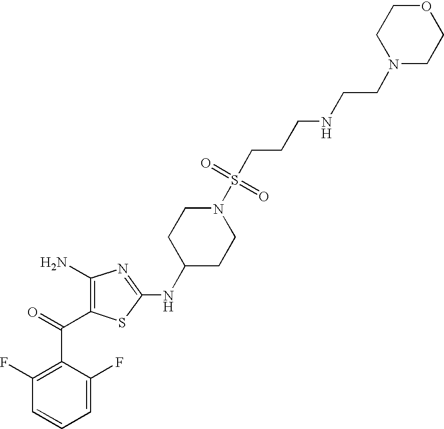 | 69 | 3 |
| V11 | 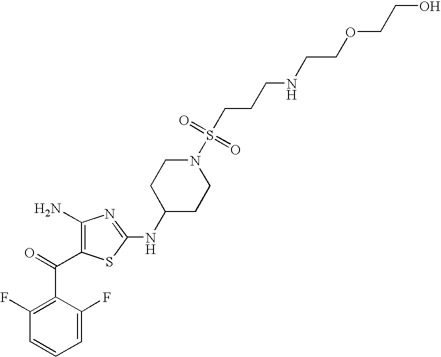 | 74 | 11 |
| V12 | 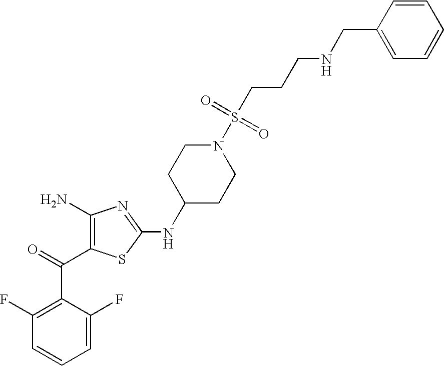 | 93 | 69 |
| V13 | 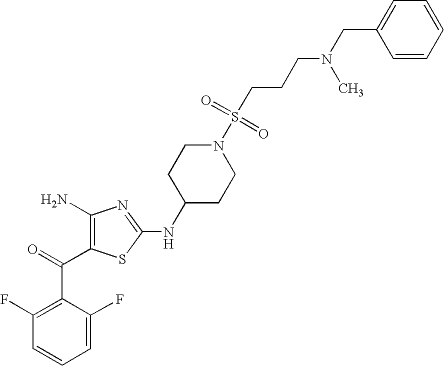 | 90 | 79 |
| V14 | 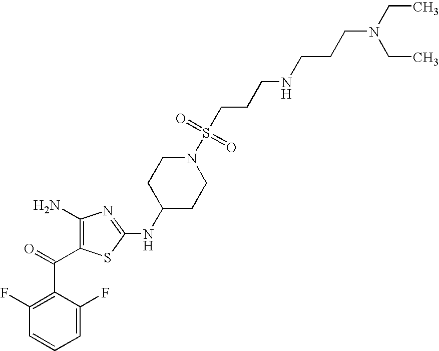 | 75 | 18 |
| V15 | 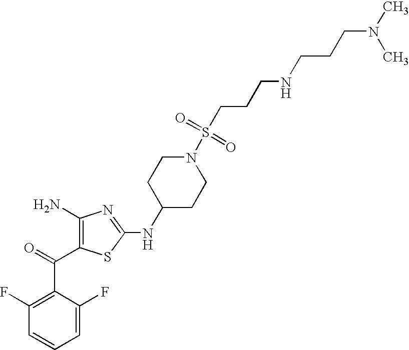 | 62 | 11 |
| V16 | 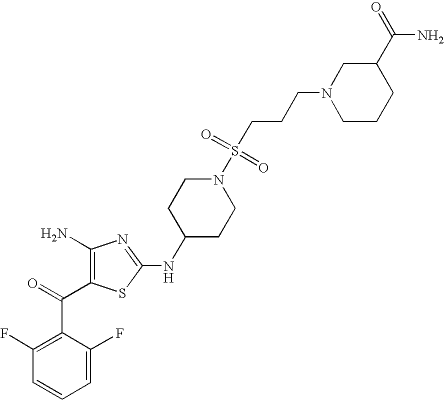 | 80 | 5 |
| V17 | 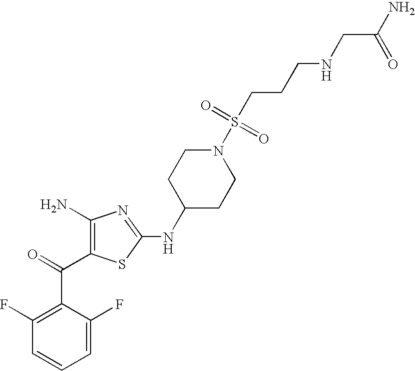 | 78 | 0 |
| V18 | 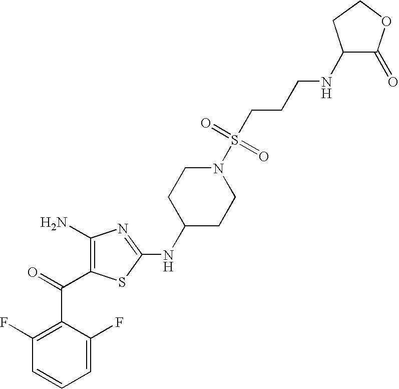 | 22 | 0 |
| V19 | 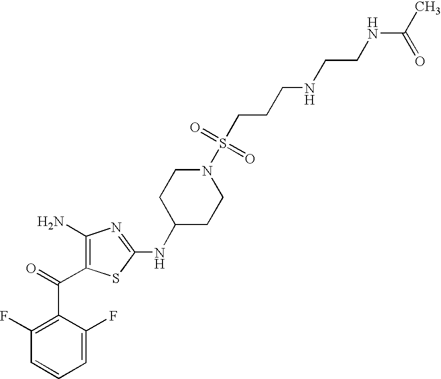 | 59 | 13 |
| V20 | 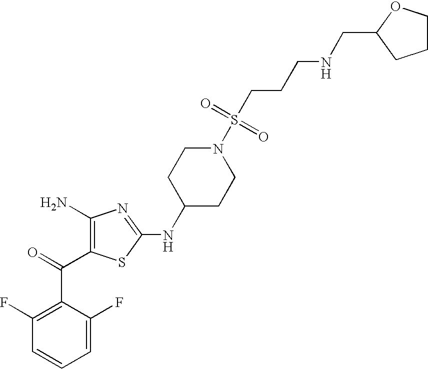 | 81 | 9 |
| V21 | 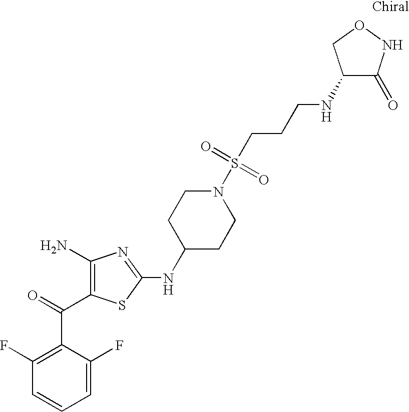 | 86 | 10 |
| V22 | 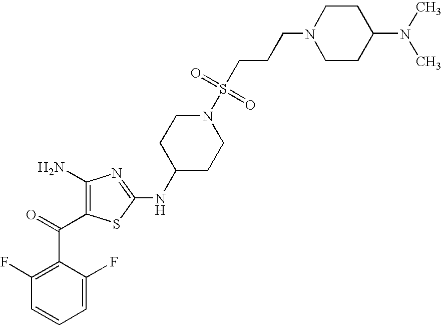 | 67 | 13 |
| V23 | 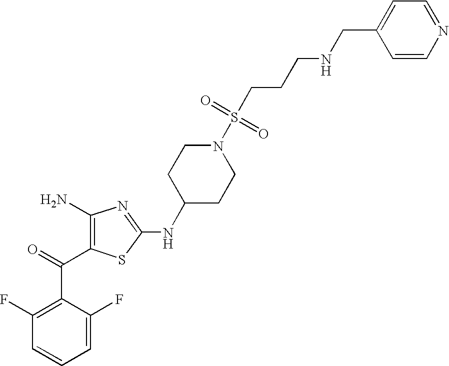 | 87 | 24 |
| V24 | 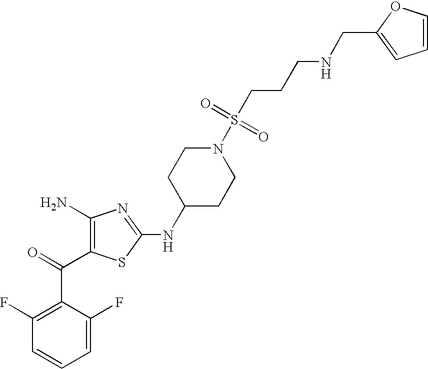 | 89 | 48 |
| V25 | 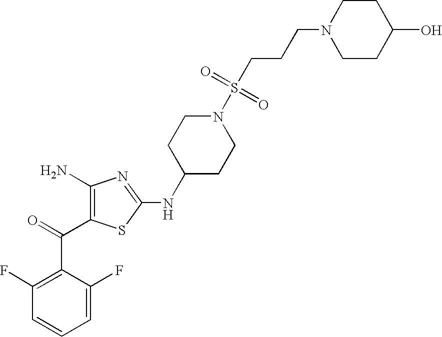 | 73 | 16 |
| V26 | 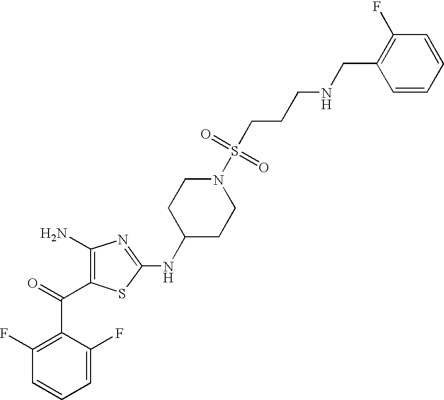 | 92 | 76 |
| V27 | 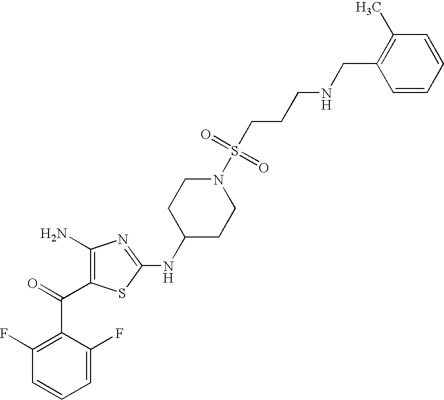 | 93 | 86 |
| V28 | 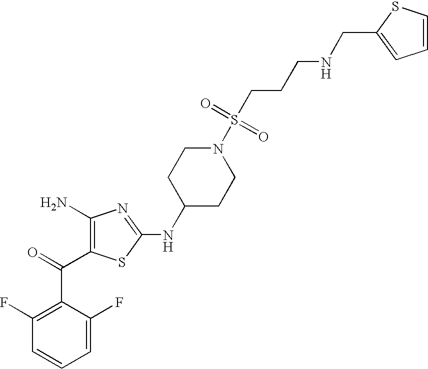 | 91 | 33 |
| V29 | 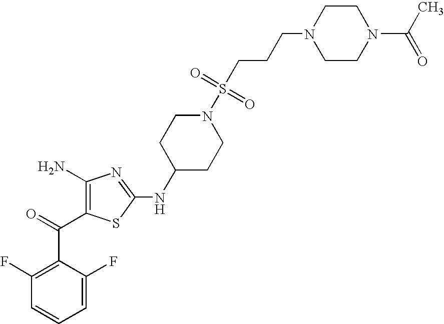 | 81 | 0 |
| V30 | 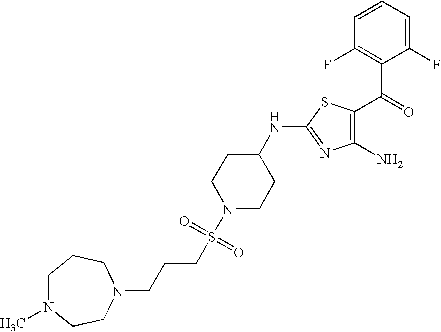 | 78 | 8 |
| V31 | 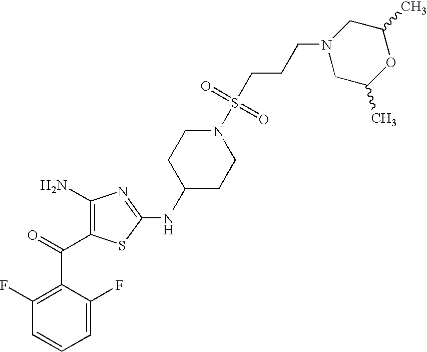 | 83 | 48 |
| V32 | 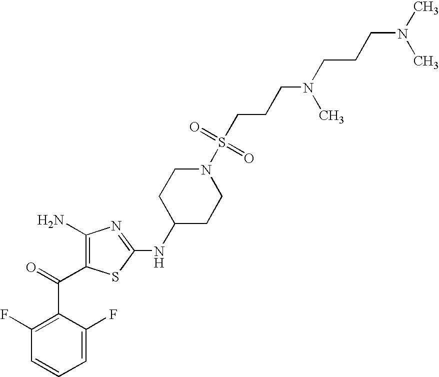 | 78 | 11 |
| V33 | 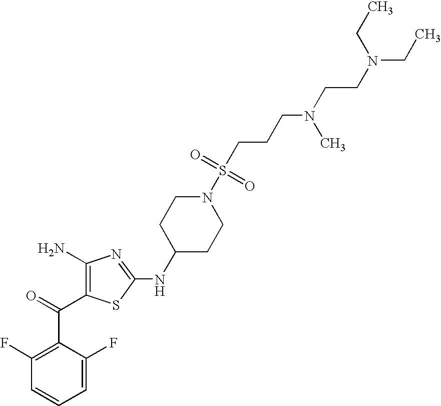 | 76 | 16 |
| V34 | 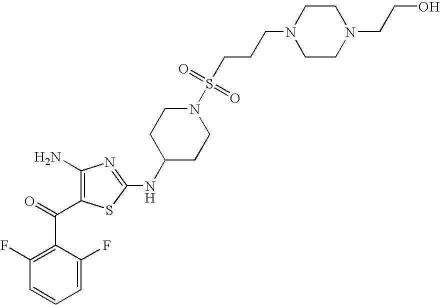 | 67 | 16 |
| V35 | 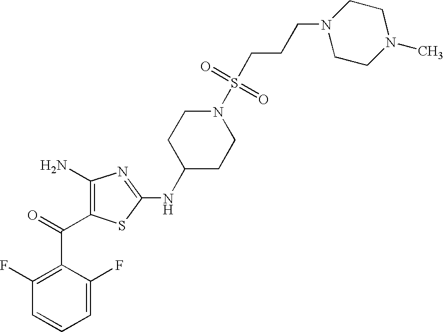 | 72 | 17 |
| V36 | 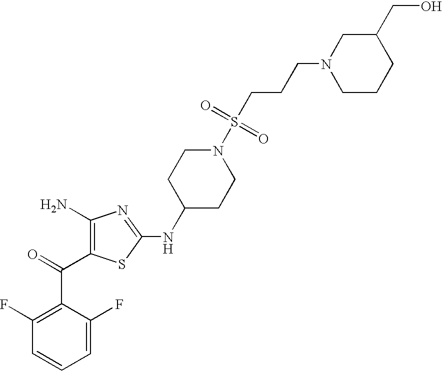 | 50 | 8 |
| V37 | 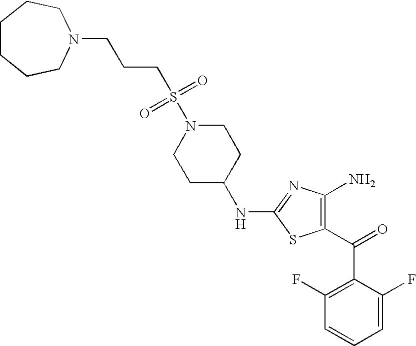 | 85 | 65 |
| V38 | 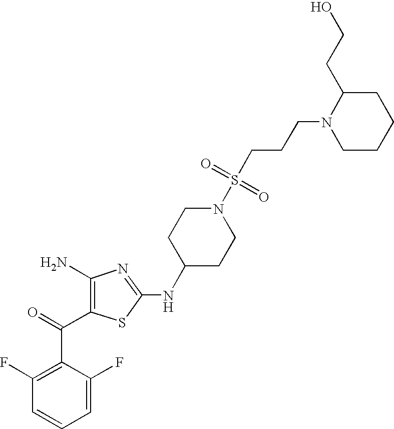 | 80 | 1 |
| V39 | 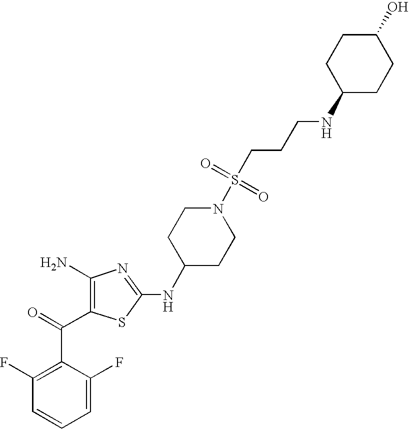 | 84 | 0 |
| V40 | 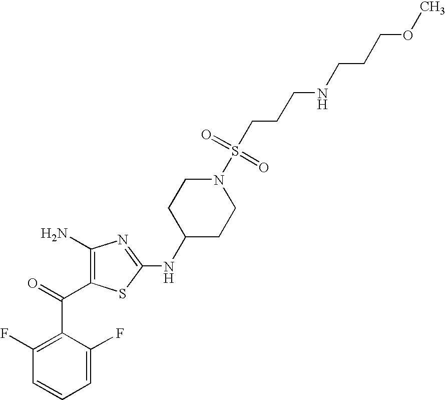 | 80 | 0 |
| V41 | 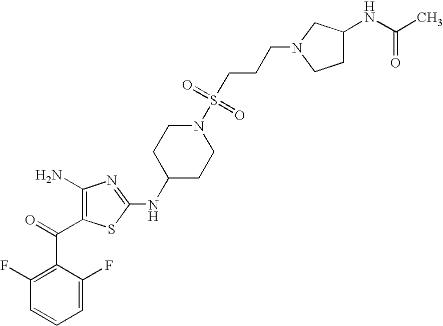 | 71 | 14 |
| V42 | 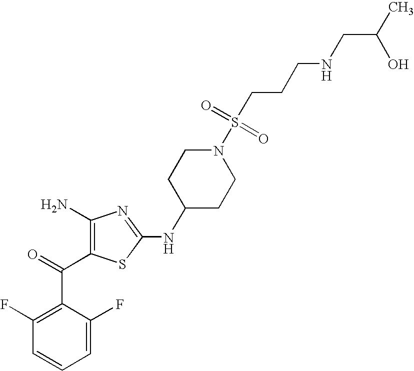 | 79 | 4 |
| V43 | 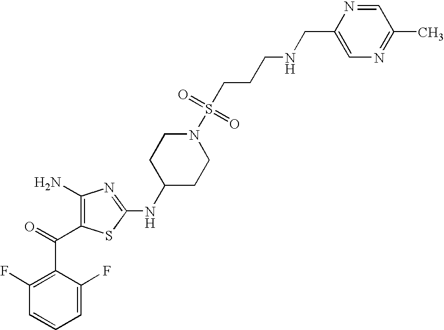 | 81 | 17 |
| V44 | 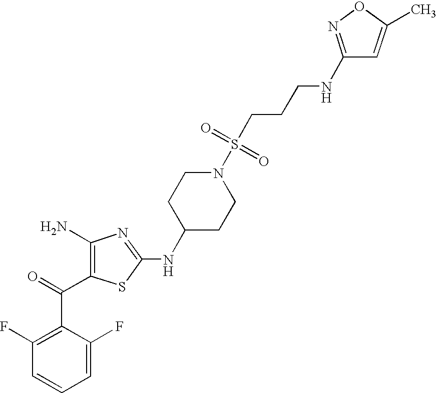 | 80 | 17 |
| V45 | 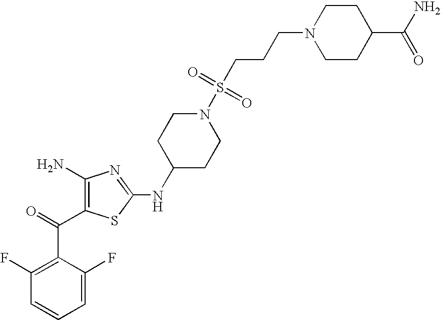 | 83 | 25 |
| V46 | 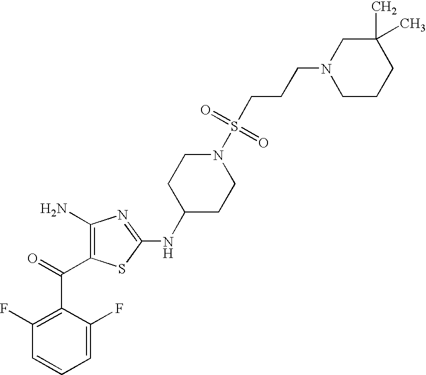 | 77 | 71 |
| V47 | 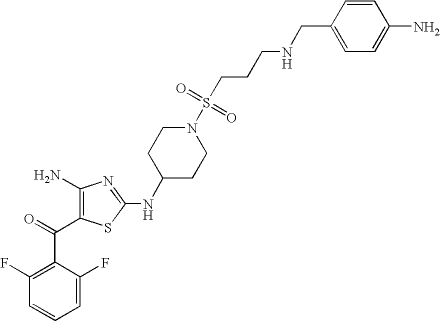 | 85 | 13 |
| V48 | 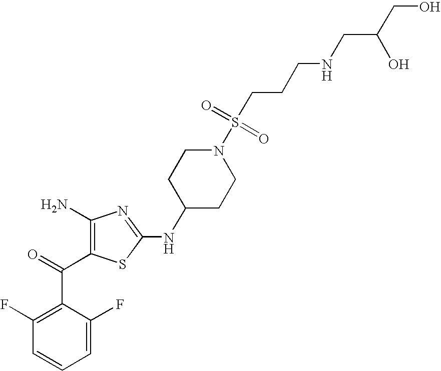 | 79 | 7 |
| V49 | 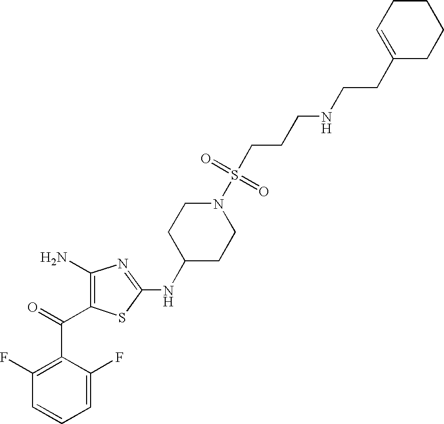 | 91 | 38 |
| V50 | 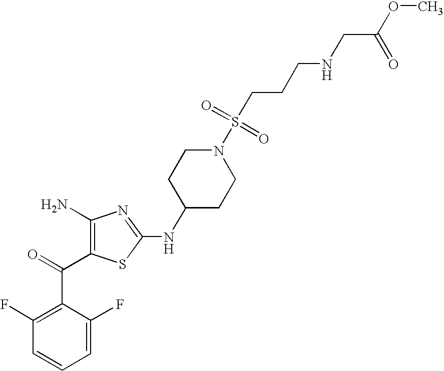 | 81 | 0 |
| V51 | 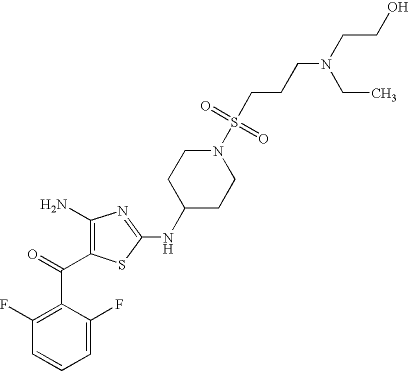 | 79 | 0 |
| V52 | 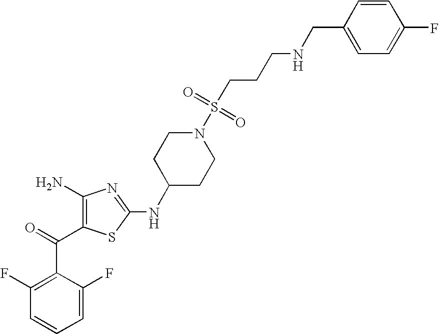 | 92 | 65 |
| V53 | 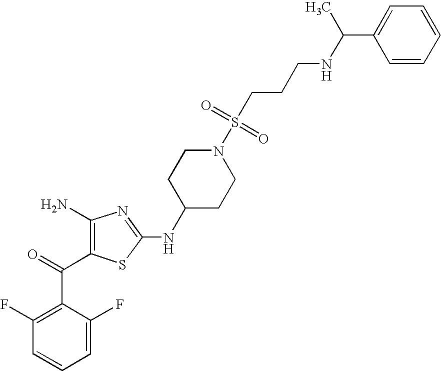 | 91 | 75 |
| V54 | 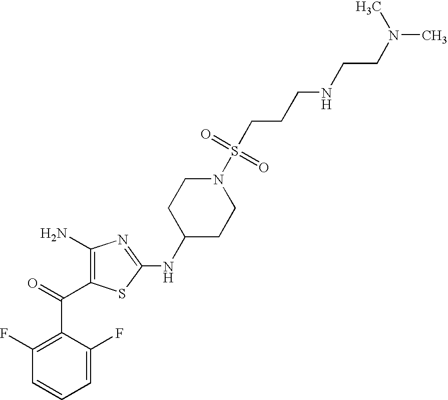 | 75 | 0 |
| V55 | 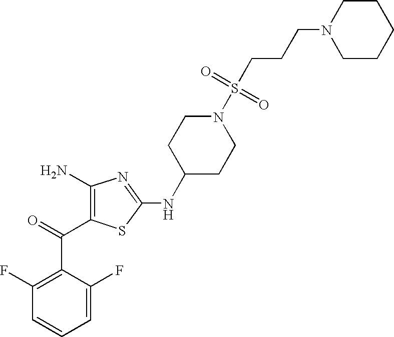 | 84 | 56 |
| V56 | 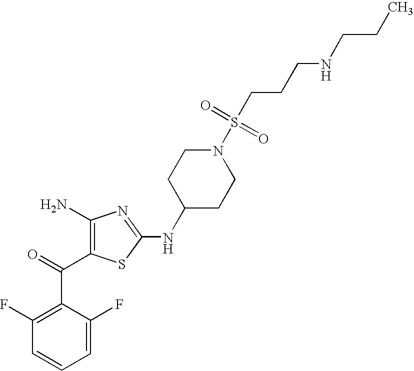 | 83 | 19 |
| V57 | 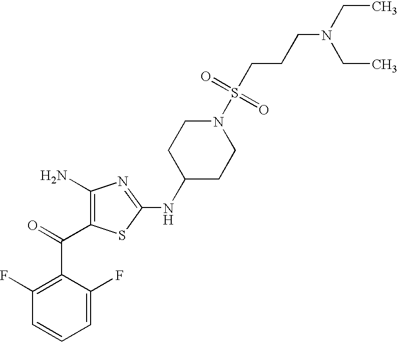 | 77 | 33 |
| V58 | 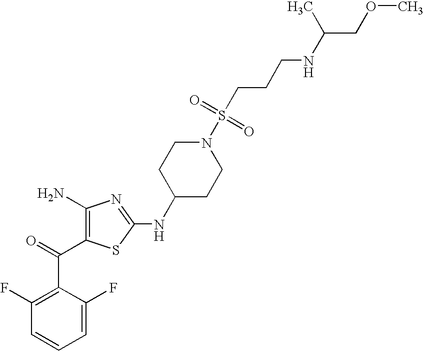 | 80 | 10 |
| V59 | 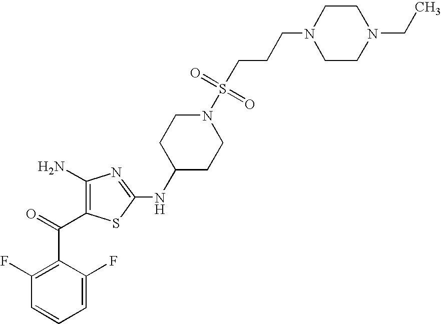 | 72 | 7 |
| V60 | 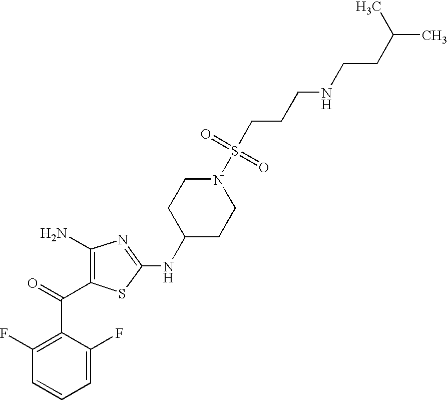 | 84 | 18 |
| V61 | 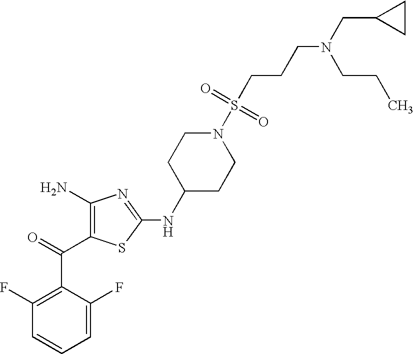 | 81 | 65 |
| V62 | 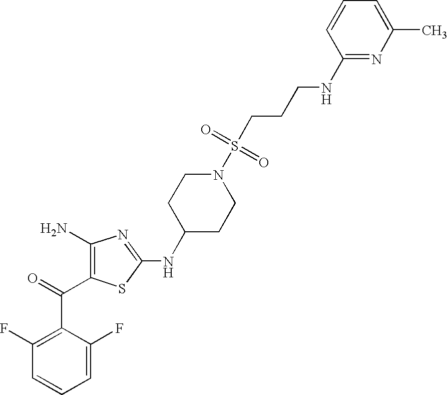 | 91 | 0 |
| V63 | 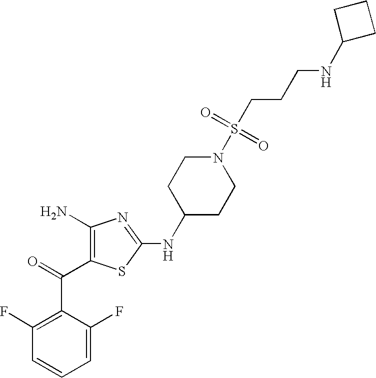 | 78 | 13 |
| V64 | 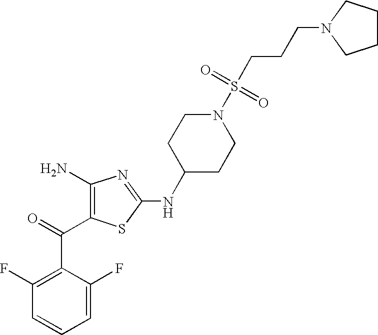 | 17 | 0 |
| V65 | 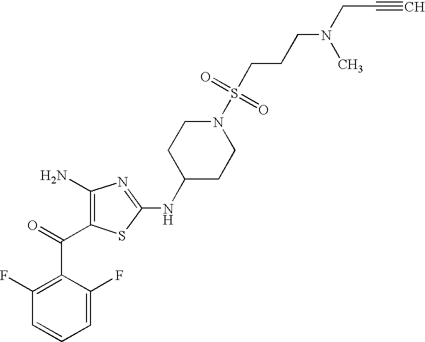 | 79 | 30 |
| V66 | 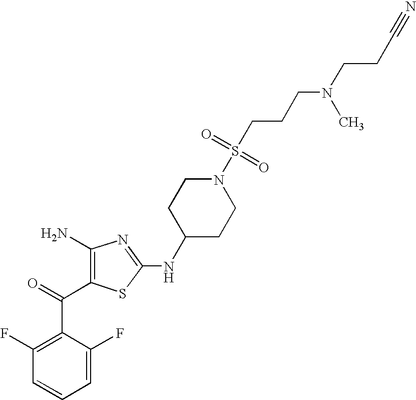 | 82 | 10 |
| V67 | 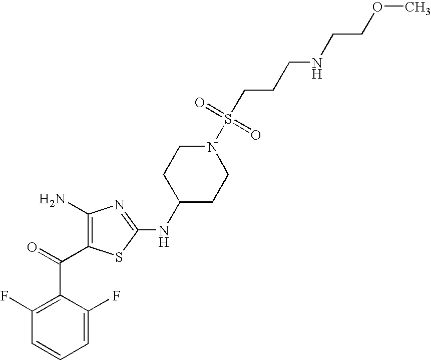 | 87 | 21 |
| V68 | 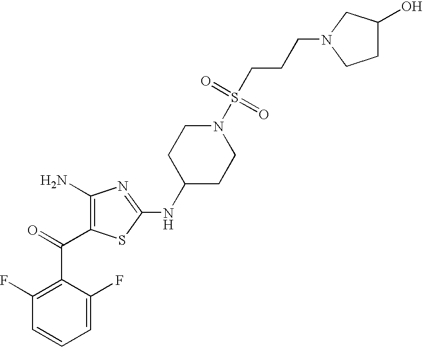 | 85 | 10 |
| V69 | 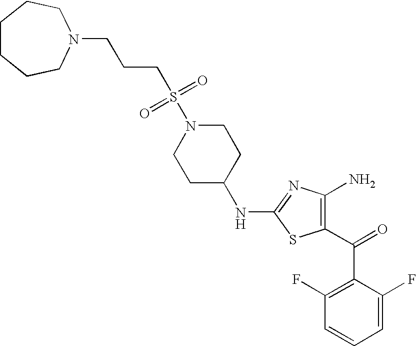 | 81 | 54 |
| V70 | 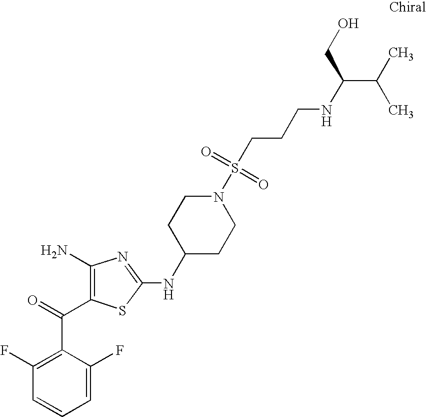 | 89 | 2 |
| V71 | 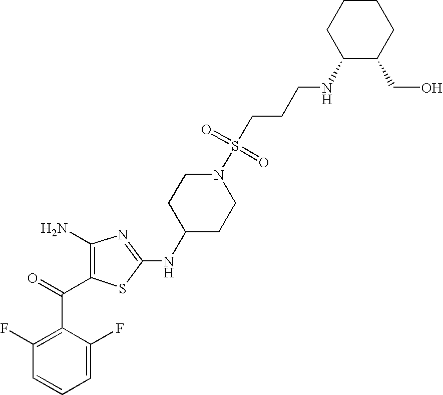 | 85 | 5 |
| V72 | 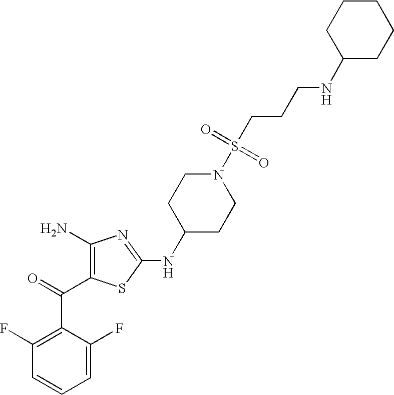 | 90 | 14 |
| V73 | 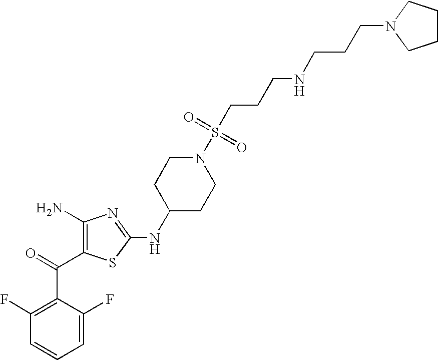 | 82 | 0 |
| V74 | 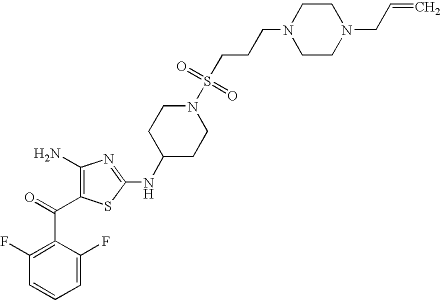 | 82 | 44 |
| V75 | 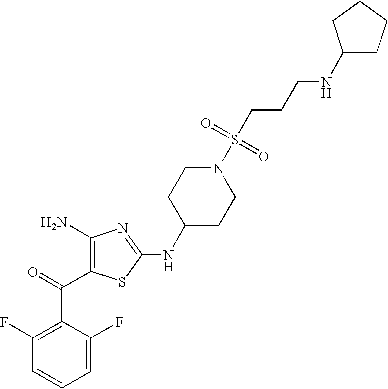 | 88 | 19 |
| V76 | 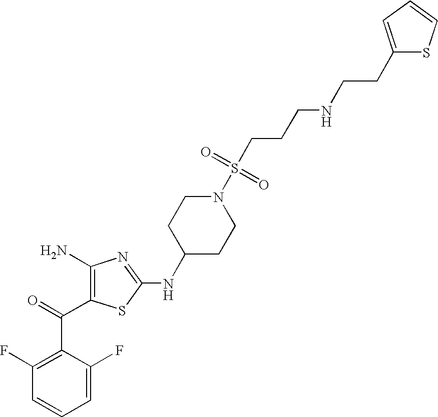 | 89 | 44 |
| V77 | 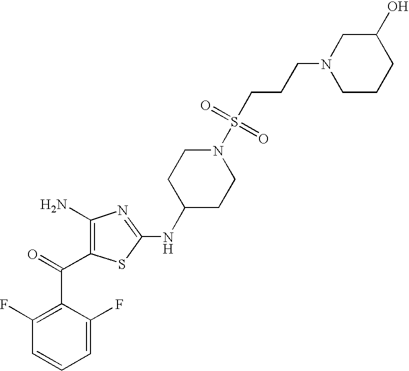 | 72 | 7 |
| V78 | 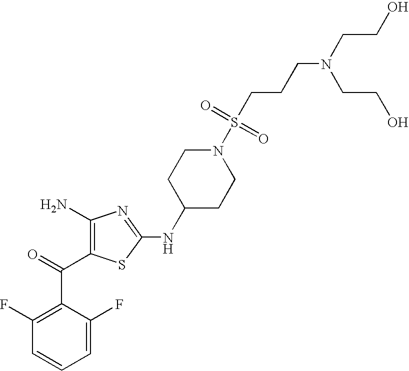 | 80 | 7 |
| V79 | 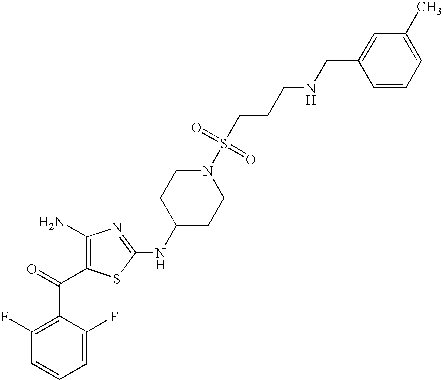 | 93 | 76 |
| V80 | 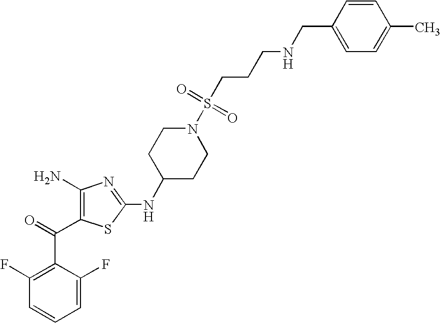 | 91 | 21 |
| V81 | 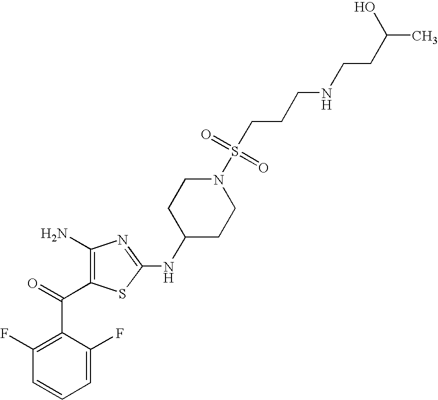 | 78 | 5 |
| V82 | 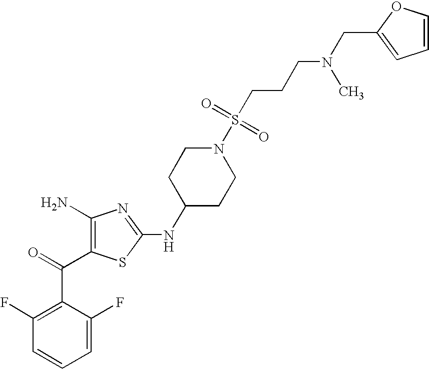 | 88 | 60 |
| V83 | 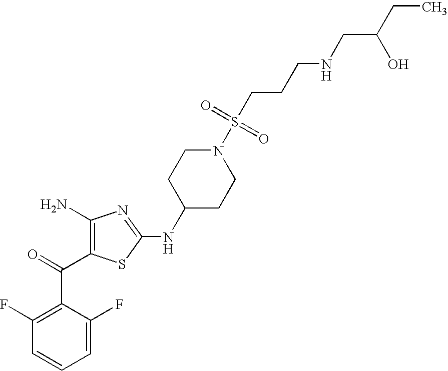 | 79 | 0 |
| V84 | 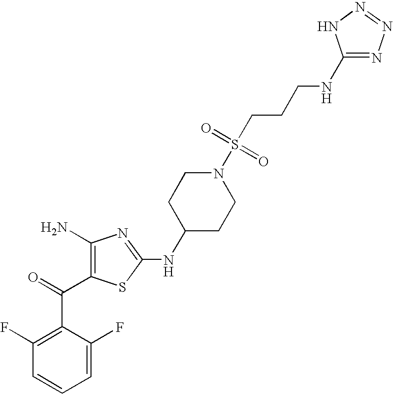 | 24 | 0 |
| V85 | 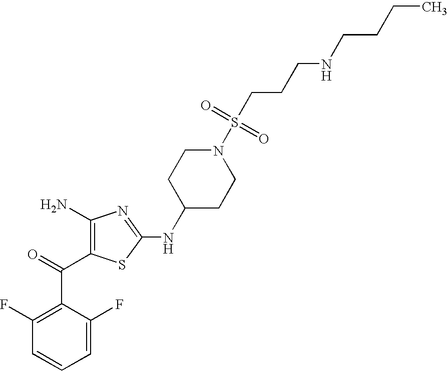 | 82 | 29 |
| V86 | 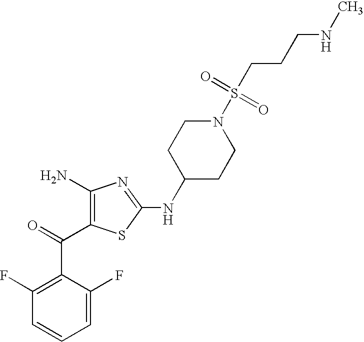 | 61 | 22 |
| V87 | 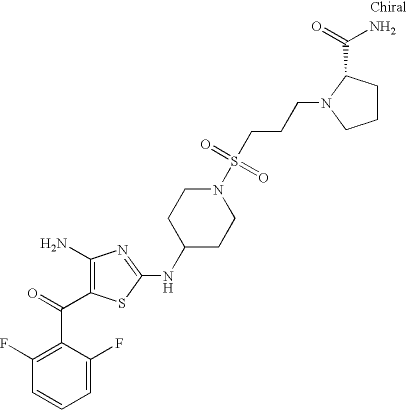 | 80 | 3 |
| V88 | 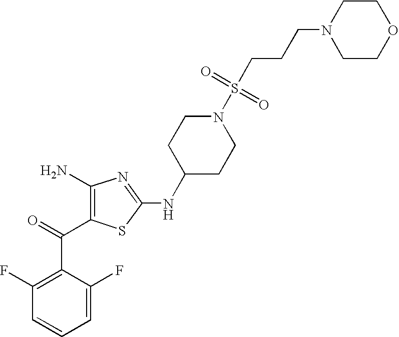 | 77 | 17 |
| V89 | 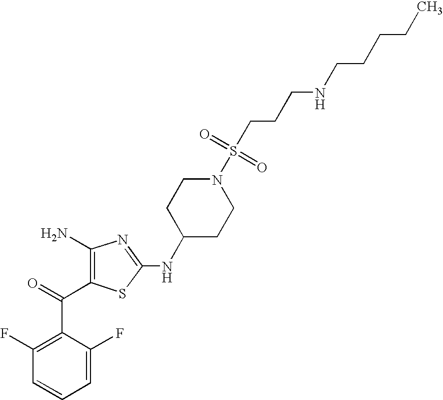 | 85 | 42 |
| V90 | 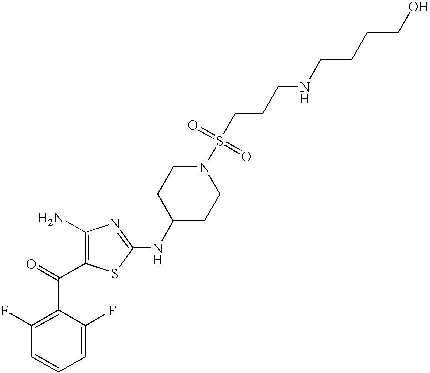 | 79 | 21 |
| V91 | 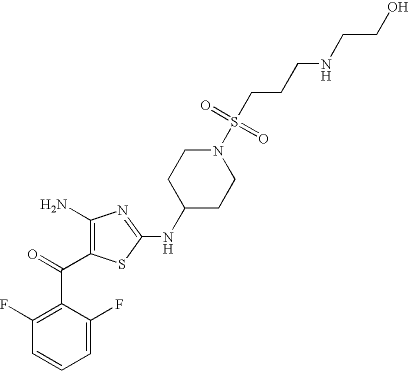 | 81 | 13 |
| V92 | 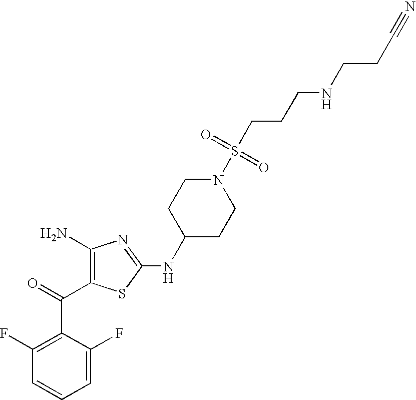 | 81 | 9 |
| V93 | 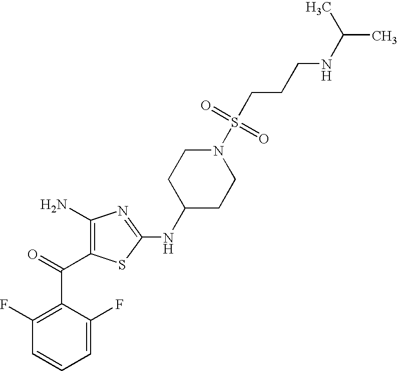 | 84 | 11 |
| V94 | 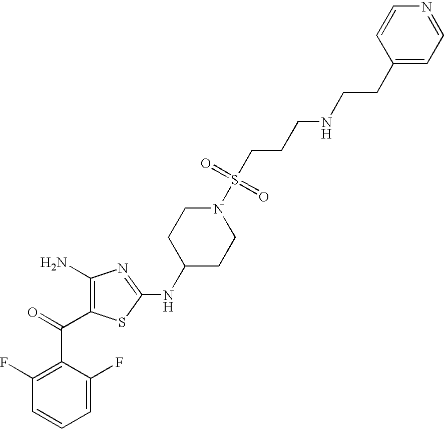 | 87 | 0 |
| V95 | 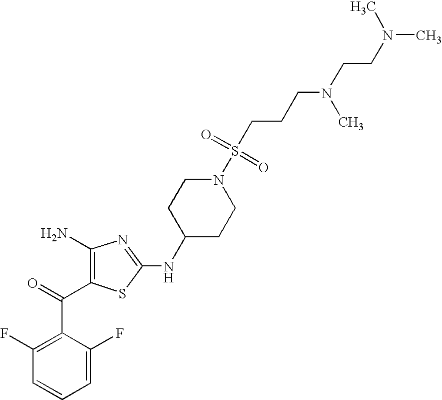 | 70 | 3 |
| V96 | 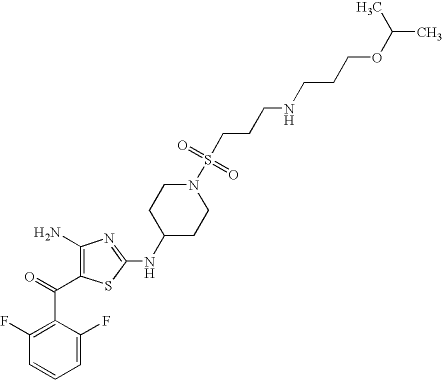 | 84 | 10 |
| V97 | 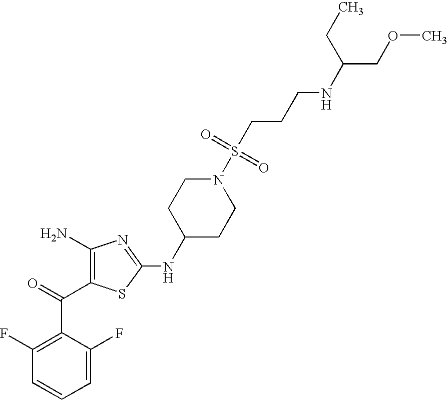 | 81 | 28 |
| V98 | 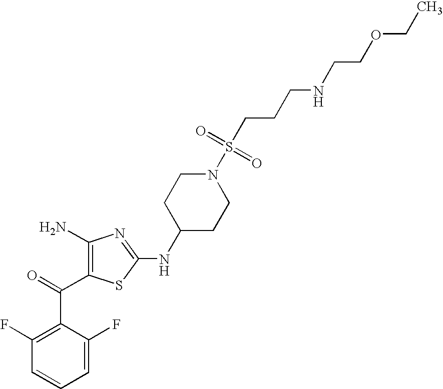 | 81 | 21 |
| V99 | 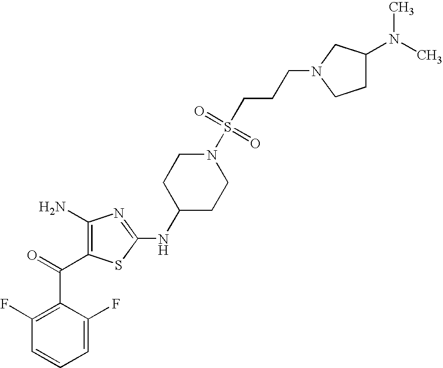 | 69 | 0 |
| V100 | 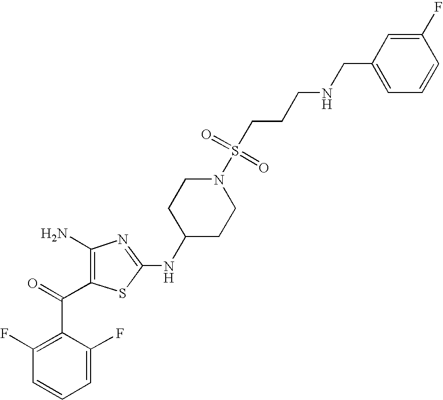 | 91 | 88 |
| V101 | 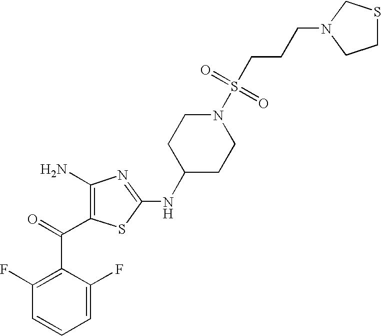 | 80 | 22 |
| V102 | 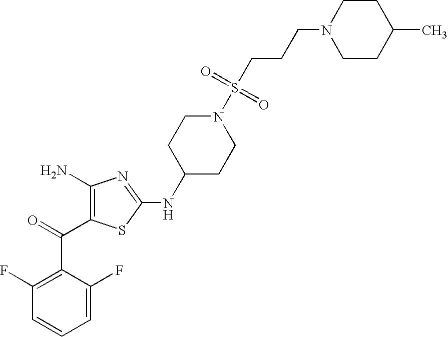 | 80 | 89 |
| V103 | 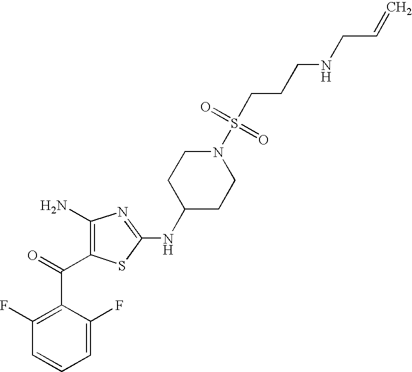 | 84 | 37 |
| V104 | 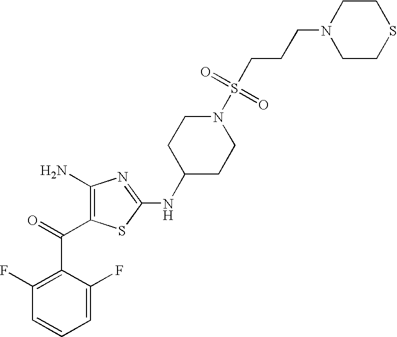 | 86 | 42 |
| V105 | 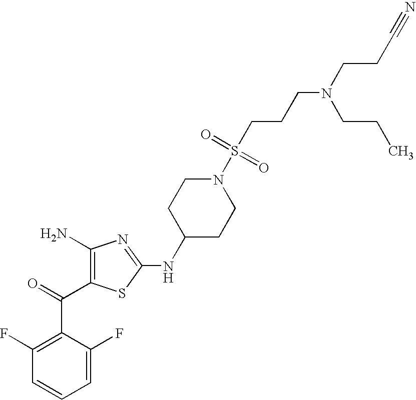 | 84 | 0 |
| V106 | 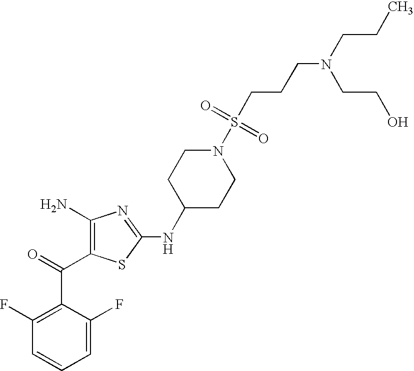 | 82 | 11 |
| V107 | 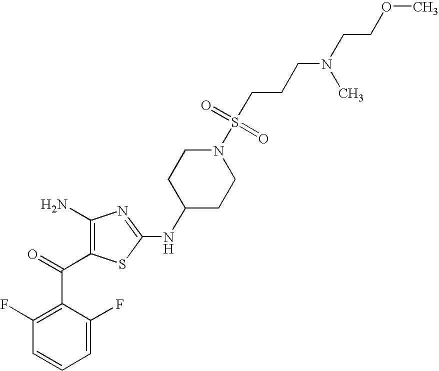 | 78 | 36 |
| V108 | 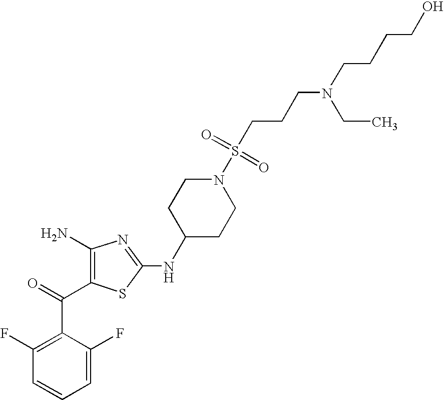 | 79 | 2 |
| V109 | 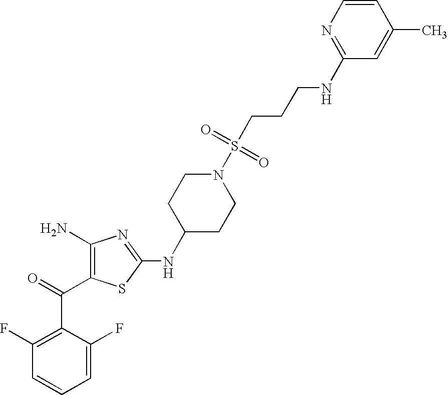 | 85 | 0 |
| V110 | 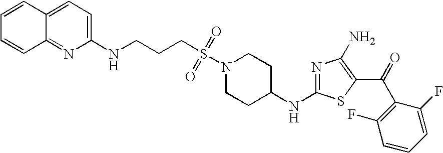 | 67 | 2 |
| V111 | 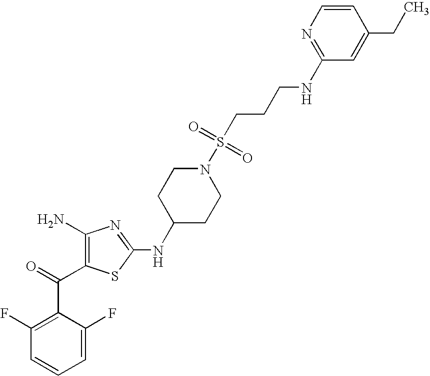 | 86 | 5 |
| V112 | 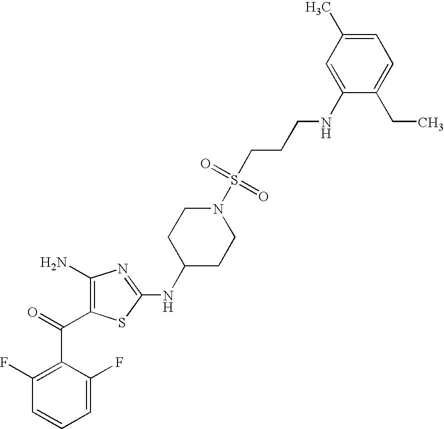 | 88 | 57 |
| V113 | 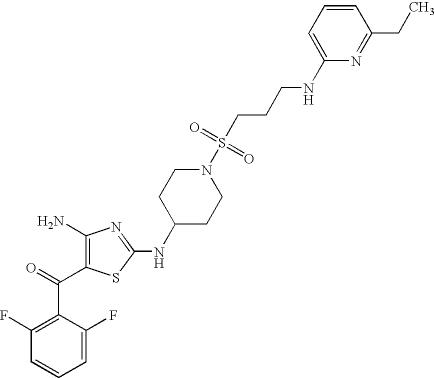 | 79 | 19 |
| V114 | 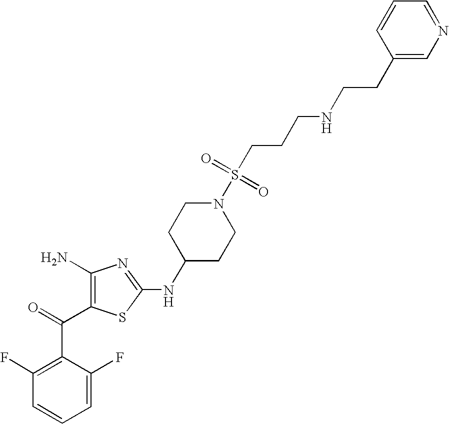 | 86 | 17 |
| V115 | 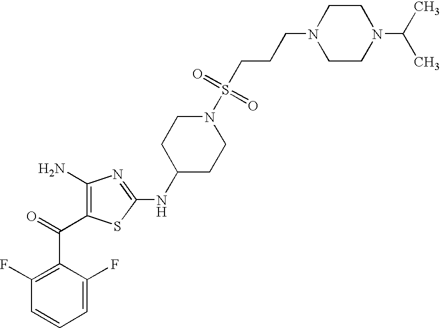 | 75 | 9 |
| V116 | 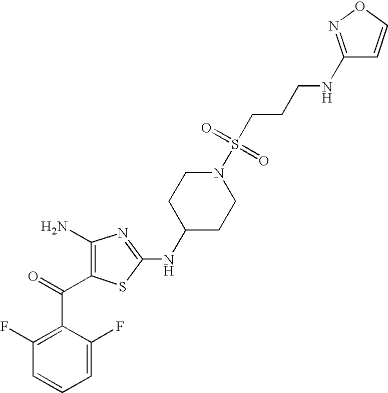 | 77 | 0 |
| V117 | 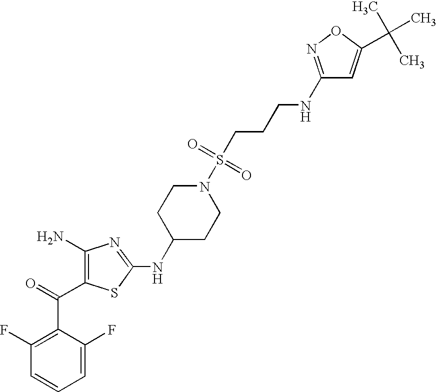 | 80 | 0 |
| V118 | 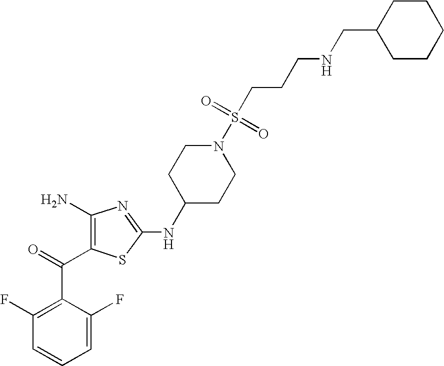 | 90 | 54 |
| V119 | 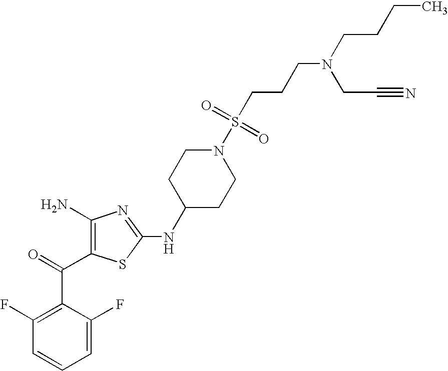 | 75 | 0 |
| V120 | 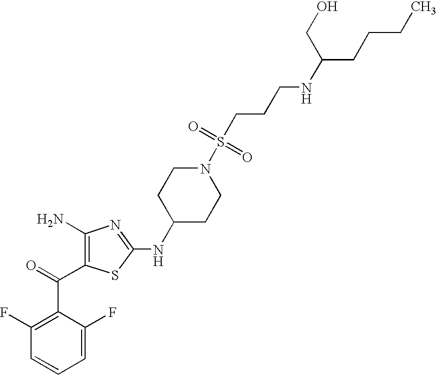 | 89 | 6 |
| V121 | 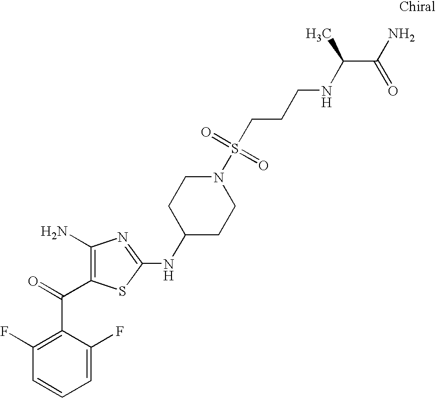 | 77 | 4 |
| V122 | 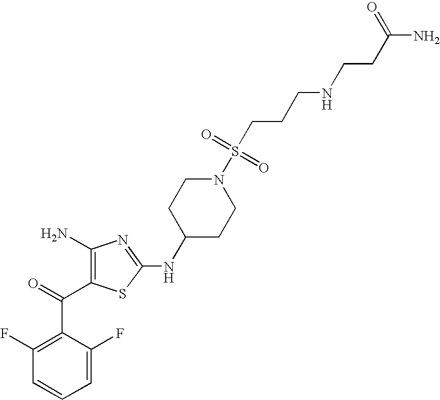 | 79 | 32 |
| V123 | 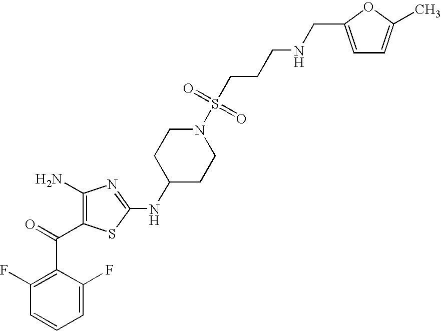 | 76 | 5 |
| V124 | 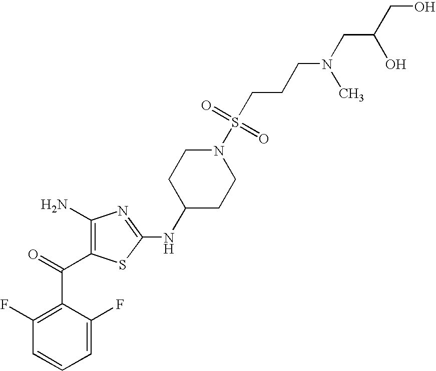 | 80 | 9 |
| V125 | 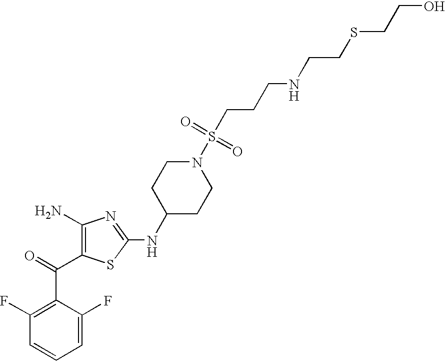 | 87 | 5 |
| V126 | 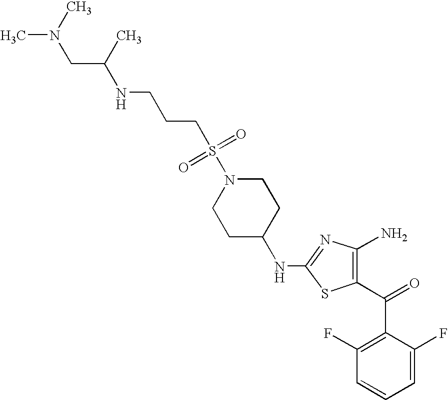 | 84 | 6 |
| V127 | 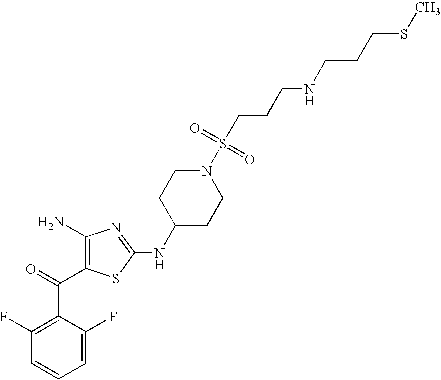 | 87 | 13 |
| V128 | 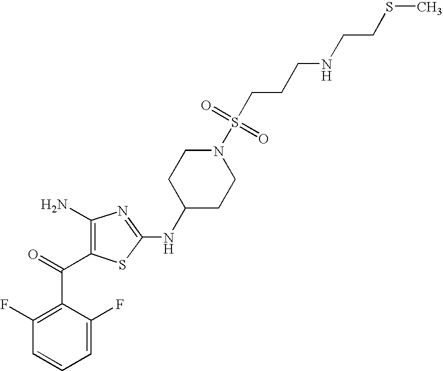 | 87 | 25 |
| V129 | 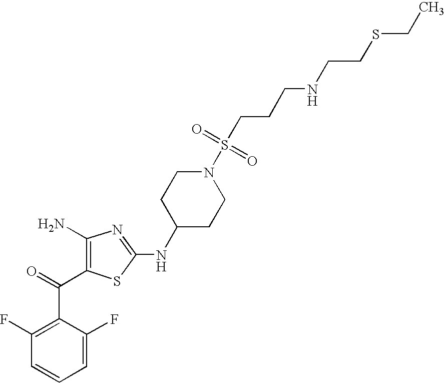 | 88 | 49 |
| V130 | 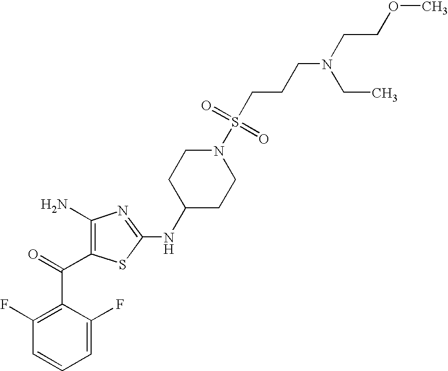 | 75 | 36 |
| V131 | 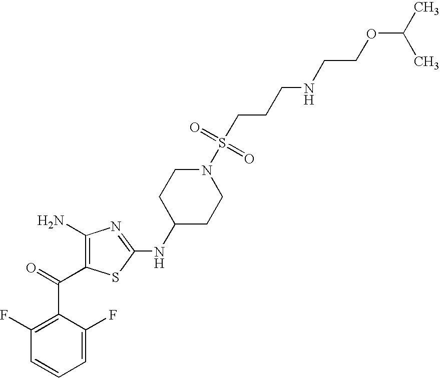 | 82 | 5 |
| V132 | 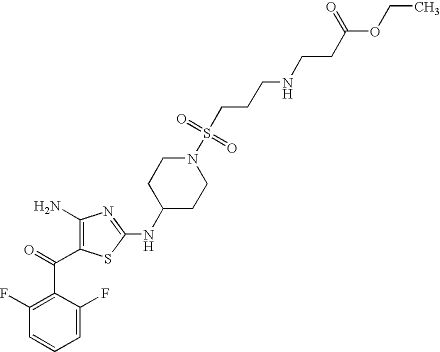 | 80 | 20 |
| V133 | 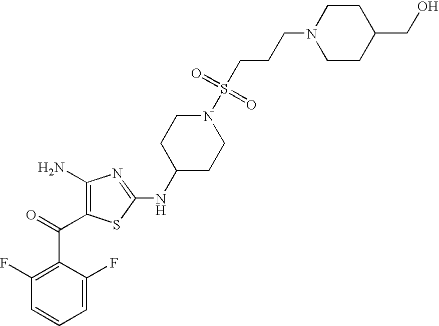 | 71 | 0 |
| V134 | 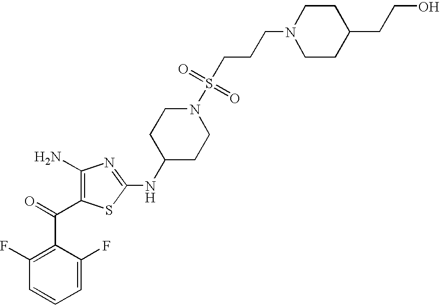 | 80 | 8 |
| V135 | 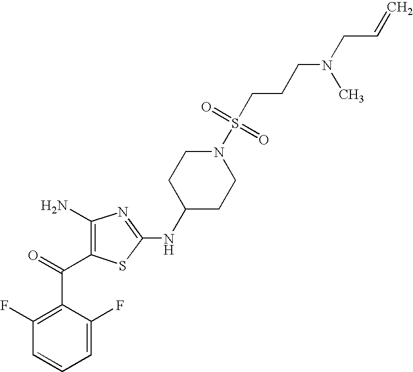 | 78 | 82 |
| V136 | 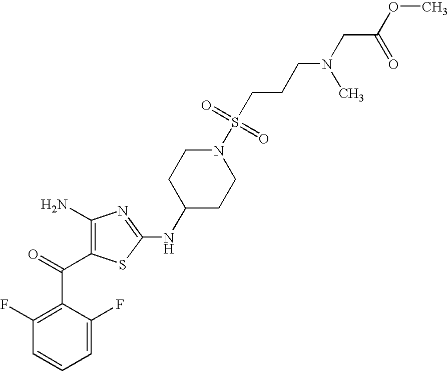 | 76 | 9 |
| V137 | 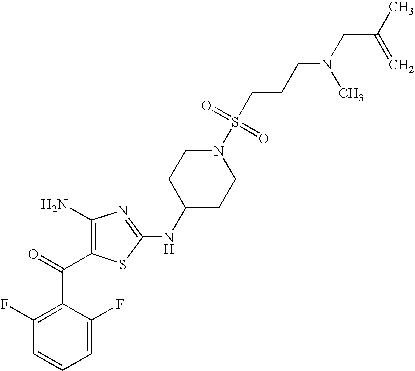 | 76 | 78 |
| V138 | 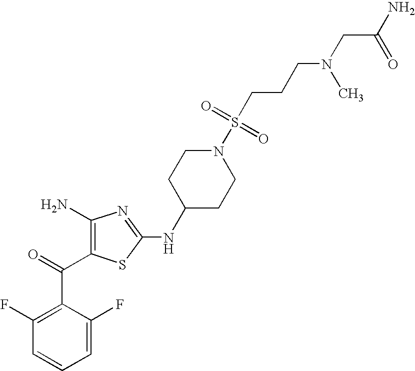 | 76 | 0 |
| V139 | 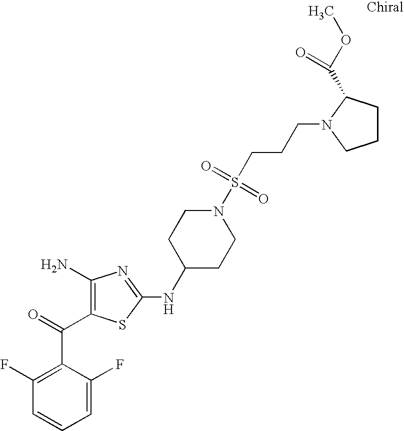 | 79 | 0 |
| V140 | 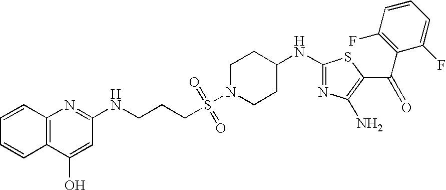 | 93 | 0 |
| V141 | 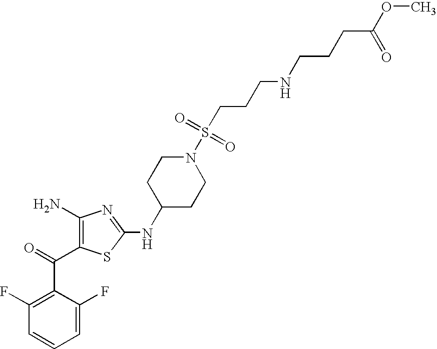 | 82 | 22 |
| V142 | 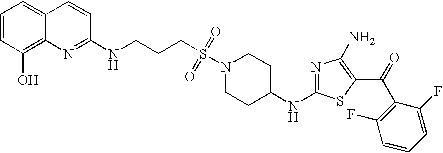 | 80 | 0 |
| V143 | 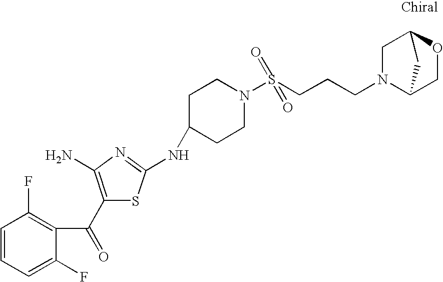 | 90 | 34 |
| V144 | 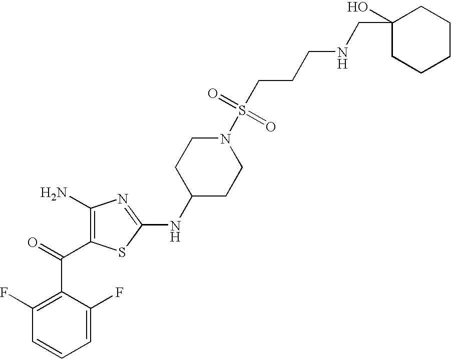 | 87 | 0 |
| V145 | 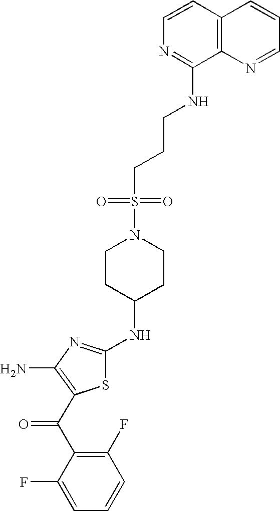 | 83 | 0 |
| V146 | 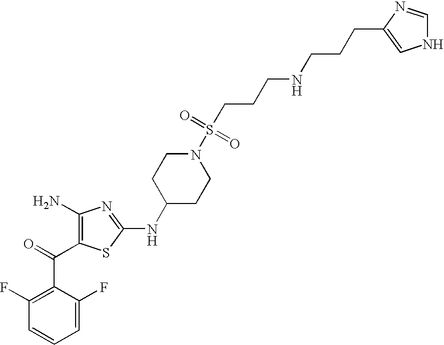 | 82 | 13 |
| V147 | 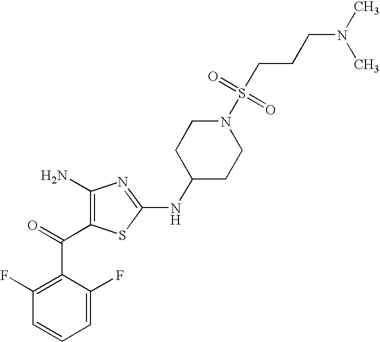 | 78 | 23 |
| V148 | 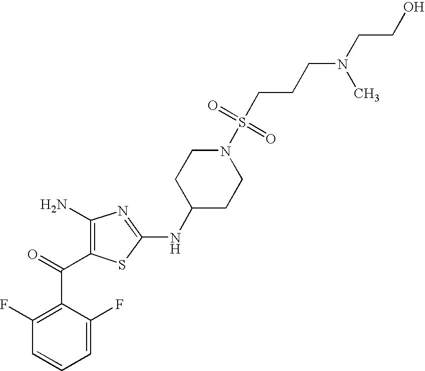 | 83 | 2 |
| V149 | 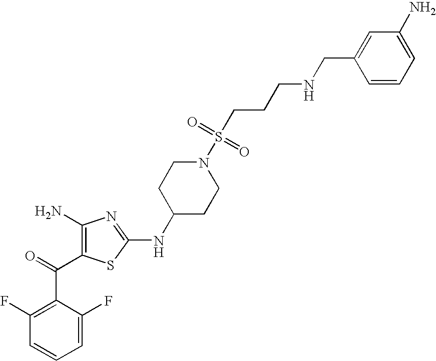 | 92 | 0 |
| V150 | 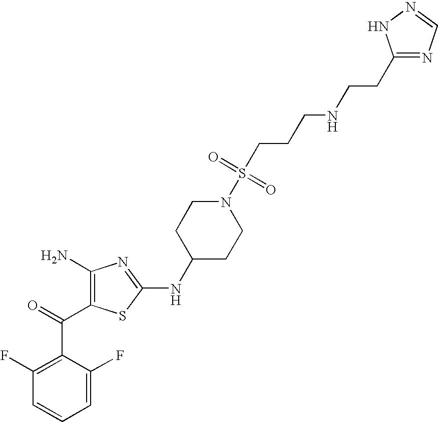 | 87 | 18 |
| V151 | 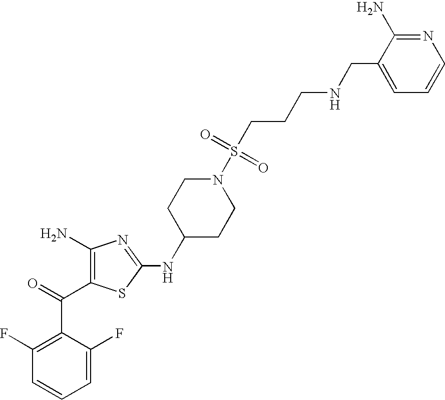 | 79 | 0 |
| V152 | 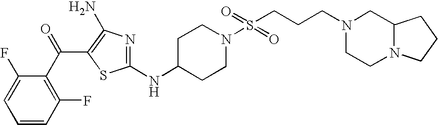 | 78 | 0 |
| V153 | 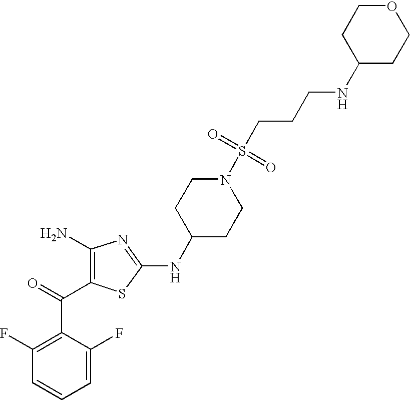 | 84 | 0 |
| V154 | 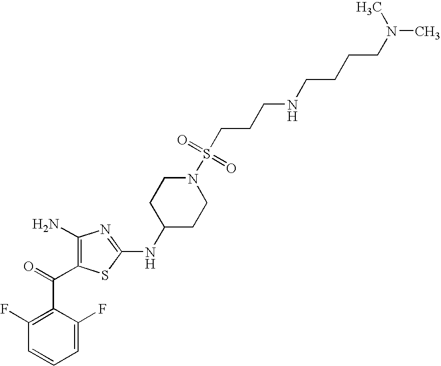 | 82 | 15 |
| V155 | 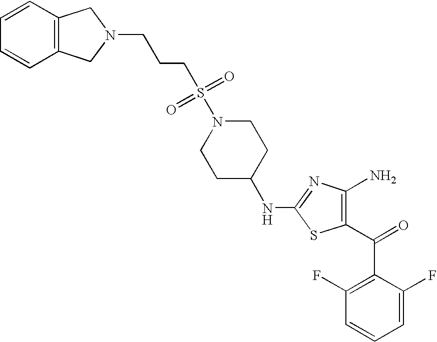 | 87 | 11 |
| V156 | 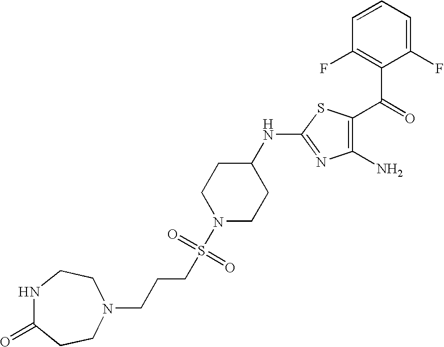 | 82 | 0 |
| V157 | 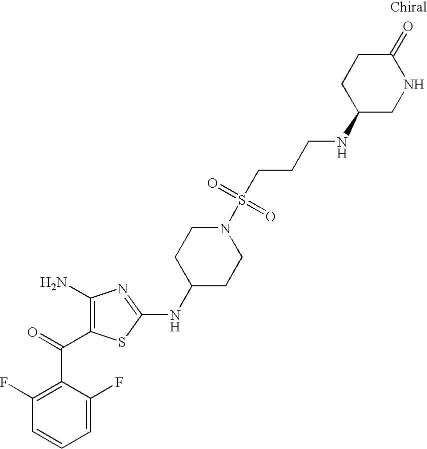 | 83 | 46 |
| V158 | 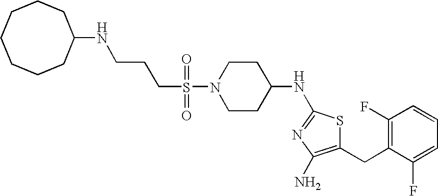 | 90 | 23 |
| V159 | 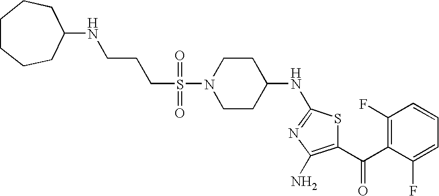 | 88 | 8 |
| V160 | 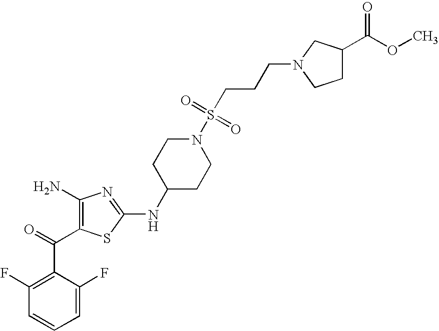 | 84 | 0 |
| V161 | 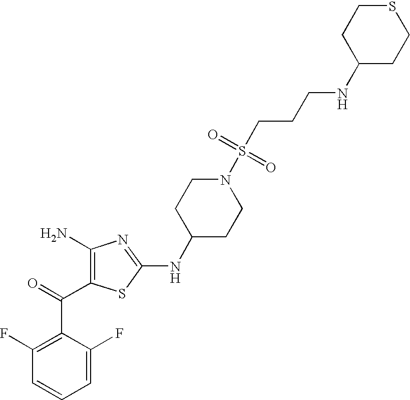 | 88 | 2 |
| V162 | 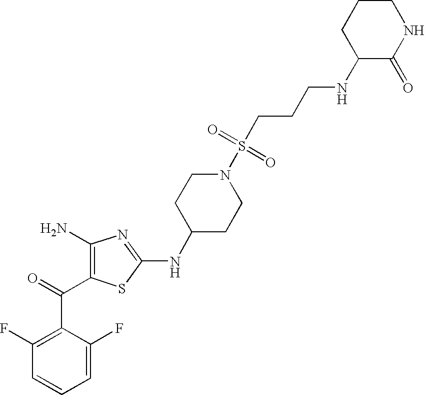 | 83 | 9 |
| V163 | 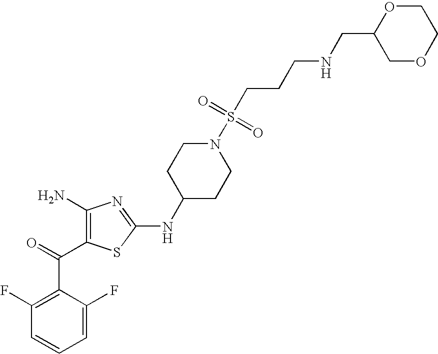 | 83 | 2 |
| V164 | 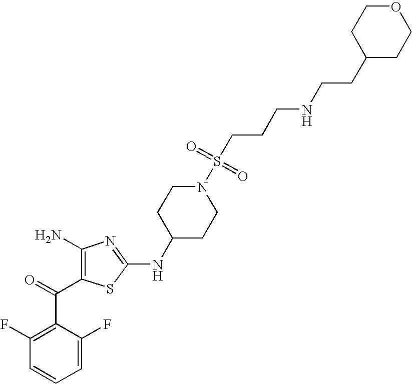 | 85 | 0 |
| V165 | 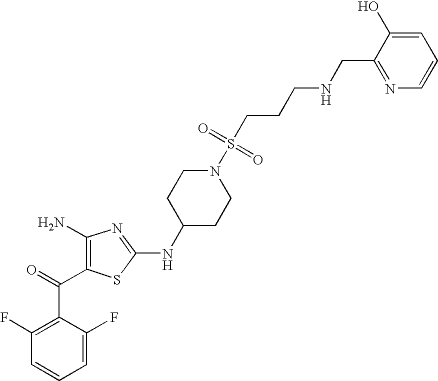 | 93 | 6 |
| V166 | 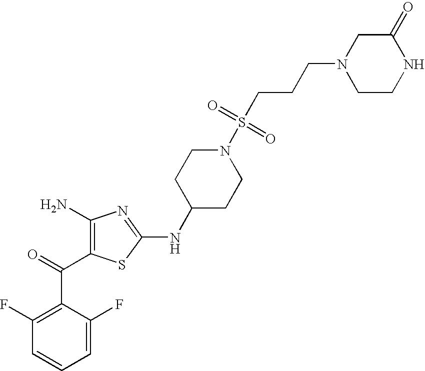 | 72 | 3 |
| V167 | 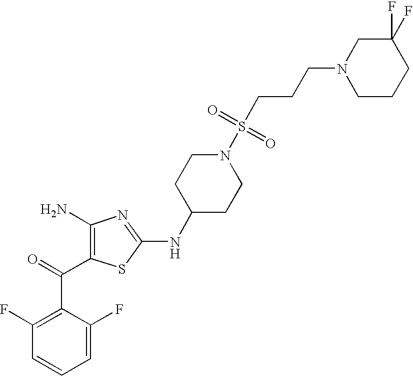 | 83 | 0 |
| V168 | 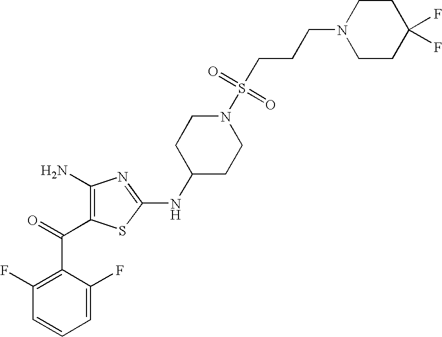 | 83 | 37 |
| V169 | 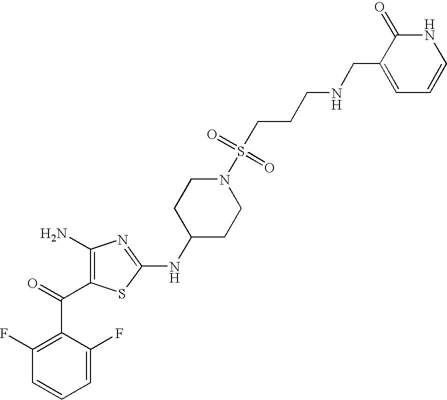 | 76 | 25 |
| V170 | 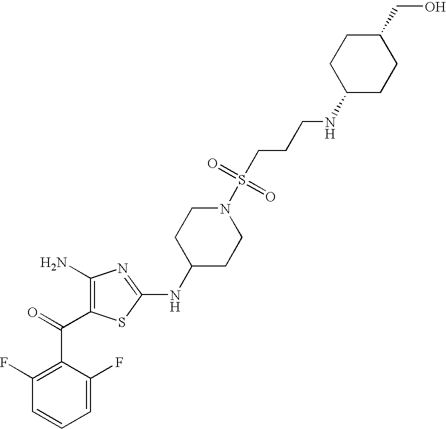 | 82 | 14 |
| V171 | 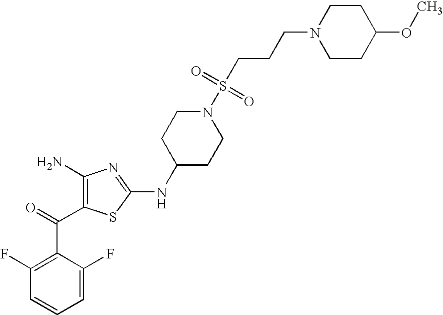 | 81 | 65 |
| V172 | 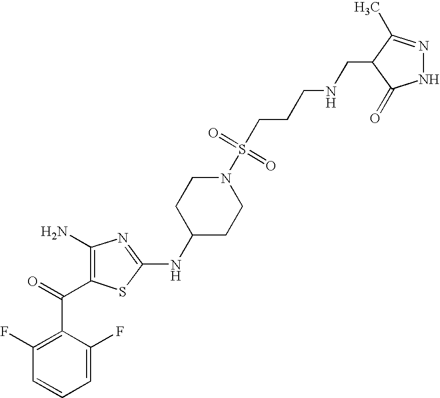 | 81 | 11 |
| V173 | 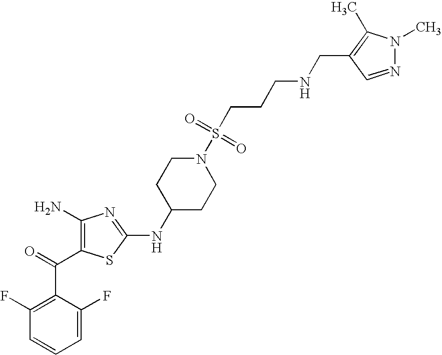 | 85 | 0 |
| V174 | 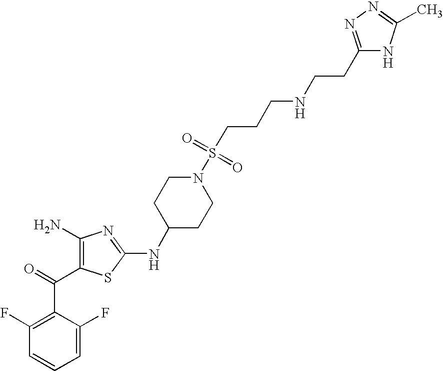 | 84 | 26 |
| V175 | 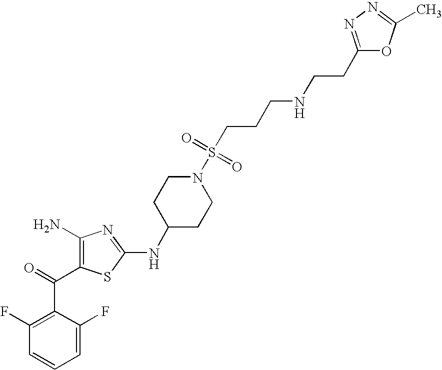 | 78 | 5 |
| V176 | 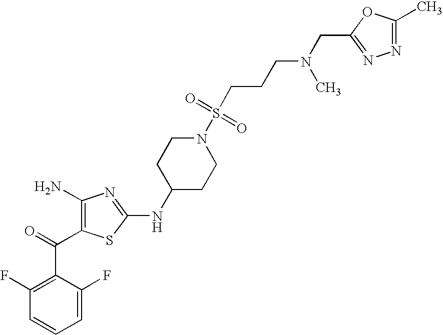 | 73 | 0 |

1. A compound or a pharmaceutically acceptable salt represented by Formula (I): 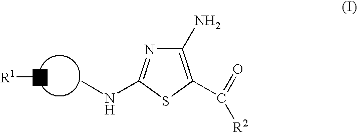
wherein: 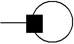
is a nitrogen-containing 3-to 10-membered heterocyclyl ring optionally substituted by one to three substituents selected from R⁷;
R¹ is:
i) R⁴;
ii) a group having a formula —SOₙ-T-(CR⁹R¹⁰)_{b}R³, —SOₙ—(CR⁹R¹⁰)_{b}-T-R³, —SOₙNR⁴C(O)R³, wherein n or b are, independently, 0, 1 or 2 and T is a bond, —O—, —NR⁴—, or —S—; or
iii) a group having a formula —C(═O)—R³, —C(═O)—HC═CH—R³, —C(═O)NHR³, —C(═O)NR⁵R⁶, or —C(═S)R³;
R² is (C₁-C₈)alkyl, (C₃-C₁₀)cycloalkyl, —O—(C₁-C₈)alkyl, (C₆-C₁₀)aryl, or 4- to 10-membered heterocyclyl, optionally substituted by one to four substituents selected from R⁷;
wherein R³ is OH, F, Cl, Br, I, CN, CF₃, NO₂, —(CH₂)_{d}NR⁵R⁶, —O—R⁴, —SOₚ—R⁴ wherein p is 0, 1, or 2, —POₚ—R⁴ wherein p is 3 or 4, (C₁-C₈)alkyl, —(CH₂)_{d}(C₃-C₁₃)cycloalkyl, —O—(C₁-C₈)alkyl, —(CH₂)_{d}—(C₆-C₁₀)aryl, —(CH₂)_{d}-(4- to 10-membered heterocyclyl), (C₂-C₆)alkenyl, (C₂-C₆)alkynyl, —SO_{q}—NR⁵R⁶, wherein d is an integer 0 to 6 and q is 1 or 2, —C(═O)—R⁸, —C(O)OR⁸, —C(═O)—NR⁵R⁶;
wherein R⁴ is selected from the group consisting of hydrogen, (C₁-C₈)alkyl, (C₂-C₆)alkenyl, (C₂-C₆)alkynyl, —O—(C₁-C₈)alkyl, —(CH₂)ₑ—(C₃-C₁₃)cycloalkyl, —(CH₂)ₑ—(C₆-C₁₀)aryl, or —(CH₂)ₑ-(4- to 10-membered heterocyclyl);
wherein R₅ is independently H or (C₁-C₈)alkyl;
wherein R⁶ is selected from the group consisting of —Si(CH₃)₃, (C₁-C₈)alkyl, —O—(C₁-C₈)alkyl, —CH₂—(C═O)—O—(C₁-C₈)alkyl, (C₃-C₁₀)cycloalkyl, (C₆-C₁₀)aryl, and 4- to 10-membered heterocyclyl; or R⁵ and R⁶ when attached to the same nitrogen may optionally be taken together with the same nitrogen to form a 5- to 10-membered heterocyclyl ring;
wherein each (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl, in the above definitions of said R³, R⁴, R⁵, R⁶ and R⁸ may be optionally substituted by one to four R⁷ substituents;
wherein R⁷ is (C₁-C₈)alkyl, (C₃-C₁₃)cycloalkyl, (C₆-C₁₀)aryl, 4- to 10-membered heterocyclyl, (C₂-C₆) alkenyl, (C₂-C₆) alkynyl, —O—(C₁-C₈)alkyl, H, OH, F, Cl, Br, I, CN, CF₃, amidino, —C(O)OR⁹, —C(O)R⁹, —SR⁹, —SO₂R⁹, —NO₂, —NR⁹C(O)R¹⁰, —OC(O)R⁹-aryl, —NSO₂R⁹, —SC(O)R⁹, —NC(═S)NR⁹R¹⁰, —O—N═CR⁹, —N═N—R⁹, —C(O)NR⁹R¹⁰, —(CH₂)ₜ—NR⁹R¹⁰, 2- to 10-membered heteroalkyl, 3- to 10-membered heteroalkenyl, 3- to 10-membered heteroalkynyl, —(CH₂)ₜ(C₆-C₁₀ aryl), —(CH₂)ₜ(4- to 10-membered heterocyclic), -(2- to 10-membered heteroalkyl)-(C₆-C₁₀ aryl), -(2- to 10-membered heteroalkyl)-(4- to 10-membered heterocyclyl), —(CH₂)ₜO(CH₂)ᵤOR⁹, and —(CH₂)ₜOR⁹, wherein t is an integer from 0 to 6 and u is an integer from 2 to 6, H or (C₁-C₈)alkyl;
wherein R⁸ is selected from the group consisting of H, OH, CF₃, (C₁-C₈)alkyl, (C₂-C₆)alkenyl, (C₂-C₆)alkynyl, —O—(C₁-C₈)alkyl, (C₃-C₁₀)cycloalkyl, —O—(C₃-C₁₀)cycloalkyl, 4- to 10-membered heterocyclyl, and 4- to 10-membered —O-heterocyclyl;
wherein each R⁹ and R¹⁰ are independently selected from the group consisting of H, (C₁-C₈)alkyl, (C₁-C₈)alkoxyl, —CH₂—(C═O)—O—(C₁-C₈)alkyl, (C₃-C₁₀)cycloalkyl, (C₆-C₁₀)aryl, and 4- to 10-membered heterocyclyl; or R⁹ and R¹⁰ when together attached to the same N, may optionally be taken together with the same nitrogen to form a 5- to 10-membered heterocyclyl ring; with the proviso that where R⁹ and R¹⁰ are both attached to the same nitrogen, then R⁹ and R¹⁰ are not both bonded to the nitrogen directly through an oxygen;
wherein any of the ring members of each (C₃-C₁₃)cycloalkyl or 4- to 10-membered heterocyclyl in R³, R⁴, R⁶, R⁷, R⁸, R⁹ and R¹⁰ may be optionally substituted with an oxo (═O) and wherein any of the (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl in R⁷, R⁹ and R¹⁰ may be independently further substituted with at least one OH, F, CL, Br, I, CN, CF₃, NO₂, —(C₁-C₈)alkyl, —(C₁-C₈) alkoxyl, COH, or C(O)—(C₁-C₈alkyl).

2. A compound or salt according to claim 1, wherein R¹ is R⁴, optionally substituted by one or more R⁹ substituents.

3. A compound or pharmaceutically acceptable salt represented by Formula (I): 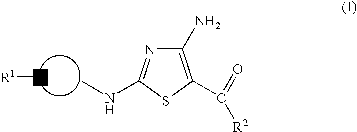
wherein: 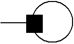
is a nitrogen-containing 3- to 10-membered heterocyclyl ring optionally substituted by one to three substituents selected from R⁷;
R¹ is a group having a formula —SOₙ-T-(CR⁹R¹⁰)_{b}R³, —SOₙ—(CR⁹R¹⁰)_{b}-T-R³, —SOₙNR⁴C(O)R³, wherein n or b are, independently, 0, 1 or 2 and T is a bond, —O—, —NR⁴—, or —S—; or
R² is (C₁-C₈)alkyl, (C₃-C₁₀)cycloalkyl, —O—(C₁-C₈)alkyl, (C₆-C₁₀)aryl, or 4- to 10-membered heterocyclyl, optionally substituted by one to four substituents selected from R⁷;
wherein R³ is OH, F, Cl, Br, I, CN, CF₃, NO₂, —NR⁵R⁶, —O—R⁴, —SOₚ—R⁴ wherein p is 0, 1, or 2, —POₚ—R⁴ wherein p is 3 or 4, (C₁-C₈)alkyl, —(CH₂)_{d}(C₃-C₁₃)cycloalkyl, —O—(C₁-C₈)alkyl, —(CH₂)_{d}—(C₆-C₁₀)aryl, —(CH₂)_{d}-(4- to 10-membered heterocyclyl), (C₂-C₆)alkenyl, (C₂-C₆)alkynyl, —SO_{q}—NR⁵R⁶, wherein d is an integer 0 to 6 and q is 1 or 2, —C(═O)—R₈, —C(O)OR⁸, or —C(═O)—NR⁵R⁶;
wherein R⁴ is each independently selected from the group consisting of hydrogen, (C₁-C₈)alkyl, (C₂-C₆)alkenyl, (C₂-C₆)alkynyl, —O—(C₁-C₈)alkyl, —(CH₂)ₑ—(C₃-C₁₃)cycloalkyl, —(CH₂)ₑ—(C₆-C₁₀)aryl, or —(CH₂)ₑ-(4- to 10-membered heterocyclyl);
wherein R⁵ is independently H or (C₁-C₈)alkyl;
wherein R⁶ is selected from the group consisting of —Si(CH₃)₃, (C₁-C₈)alkyl, —O—(C₁-C₈)alkyl, —CH₂—(C═O)—O—(C₁-C₈)alkyl, (C₃-C₁₀)cycloalkyl, (C₆-C₁₀)aryl, and 4- to 10-membered heterocyclyl; or R⁵ and R⁶ when attached to the same nitrogen may optionally be taken together with the same nitrogen to form a 5- to 10-membered heterocyclyl ring;
wherein each (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl, in the above definitions of said R³, R⁴, R⁵, R⁶ and R⁸ may be optionally substituted by one to four R⁷ substituents;
wherein R⁷ is (C₁-C₈)alkyl, (C₃-C₁₃)cycloalkyl, (C₆-C₁₀)aryl, 4- to 10-membered heterocyclyl, (C₂-C₆) alkenyl, (C₂-C₆) alkynyl, —O—(C₁-C₈)alkyl, H, OH, F, Cl, Br, I, CN, CF₃, amidino, —C(O)OR⁹, —C(O)R⁹, —SR⁹, —SO₂R⁹, —NO₂, —NR⁹C(O)R¹⁰, —OC(O)R⁹-aryl, —NSO₂R⁹, —SC(O)R⁹, —NC(═S)NR⁹R¹⁰, —O—N═CR⁹, —N═N—R⁹, —C(O)NR⁹R¹⁰, —(CH₂)ₜ—NR⁹R¹⁰, 2 to 10 membered heteroalkyl, 3- to 10-membered heteroalkenyl, 3- to 10-membered heteroalkynyl, —(CH₂)ₜ(C₆-C₁₀ aryl), —(CH₂)ₜ(4 to 10 membered heterocyclic), -(2 to 10 membered heteroalkyl)-(C₆-C₁₀ aryl), -(2 to 10 membered heteroalkyl)-(4 to 10 membered heterocyclyl), —(CH₂)ₜO(CH₂)ᵤOR⁹, and —(CH₂)ₜOR⁹, wherein t is an integer from 0 to 6 and u is an integer from 2 to 6, H or (C₁-C₈)alkyl;
wherein R⁸ is selected from the group consisting of H, OH, CF₃, (C₁-C₈)alkyl, (C₂-C₆)alkenyl, (C₂-C₆)alkynyl, —O—(C₁-C₈)alkyl, (C₃-C₁₀)cycloalkyl, —O—(C₃-C₁₀)cycloalkyl, 4- to 10-membered heterocyclyl, and 4- to 10-membered —O-heterocyclyl;
wherein each R⁹ and R¹⁰ are independently selected from the group consisting of H, (C₁-C₈)alkyl, (C₁-C₈)alkoxyl, —CH₂—(C═O)—O—(C₁-C₈)alkyl, (C₃-C₁₀)cycloalkyl, (C₆-C₁₀)aryl, and 4- to 10-membered heterocyclyl; or R₉ and R¹⁰ when together attached to the same N, may optionally be taken together with the same nitrogen to form a 5- to 10-membered heterocyclyl ring; with the proviso that where R⁹ and R¹⁰ are both attached to the same nitrogen, then R⁹ and R¹⁰ are not both bonded to the nitrogen directly through an oxygen;
wherein any of the ring members of each (C₃-C₁₃)cycloalkyl or 4- to 10-membered heterocyclyl in R³, R⁴, R⁶, R⁷, R⁸, R⁹ and R¹⁰ may be optionally substituted with an oxo (═O) and wherein any of the (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl in R⁷, R⁹ and R¹⁰ may be independently further substituted with at least one OH, F, CL, Br, I, CN, CF₃, NO₂, —(C₁-C₈)alkyl, —(C₁-C₈) alkoxyl, COH, or C(O)—(C₁-C₈alkyl).

4. A compound or pharmaceutically acceptable salt represented by Formula (I): 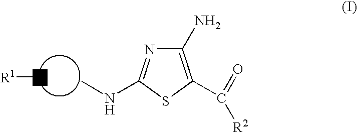
wherein: 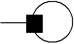
is a nitrogen-containing 3- to 10-membered heterocyclyl ring optionally substituted by one to three substituents selected from R⁷;
R¹ is a group having a formula —C(═O)—R³, —C(═O)—HC═CH—R³, —C(═O)NHR³, —C(═O)NR⁵R⁶ or —C(═S)R³;
R² is (C₁-C₈)alkyl, (C₃-C₁₀)cycloalkyl, —O—(C₁-C₈)alkyl, (C₆-C₁₀)aryl, or 4- to 10-membered heterocyclyl, optionally substituted by one to four substituents selected from R⁷;
wherein R³ is OH, F, Cl, Br, I, CN, CF₃, NO₂, —NR⁵R⁶, —O—R⁴, —SOₚ—R⁴ wherein p is 0, 1, or 2, —POₚ—R⁴ wherein p is 3 or 4, (C₁-C₈)alkyl, —(CH₂)_{d}(C₃-C₁₃)cycloalkyl, —O—(C₁-C₈)alkyl, —(CH₂)_{d}—(C₆-C₁₀)aryl, —(CH₂)_{d}-(4- to 10-membered heterocyclyl), (C₂-C₆)alkenyl, (C₂-C₆)alkynyl, —SO_{q}—NR⁵R⁶, wherein d is an integer 0 to 6 and q is 1 or 2, —C(═O)—R⁸, —C(O)OR⁸, or —C(═O)—NR⁵R⁶;
wherein R⁴ is each independently selected from the group consisting of hydrogen, (C₁-C₈)alkyl, (C₂-C₆)alkenyl, (C₂-C₆)alkynyl, —O—(C₁-C₈)alkyl, —(CH₂)ₑ—(C₃-C₁₃)cycloalkyl, —(CH₂)ₑ—(C₆-C₁₀)aryl, or —(CH₂)ₑ-(4- to 10-membered heterocyclyl);
wherein R⁵ is independently H or (C₁-C₈)alkyl;
wherein R⁶ is selected from the group consisting of —Si(CH₃)₃, (C₁-C₈)alkyl, —O—(C₁-C₈)alkyl, —CH₂—(C═O)—O—(C₁-C₈)alkyl, (C₃-C₁₀)cycloalkyl, (C₆-C₁₀)aryl, and 4- to 10-membered heterocyclyl; or R⁵ and R⁶ when attached to the same nitrogen may optionally be taken together with the same nitrogen to form a 5- to 10-membered heterocyclyl ring;
wherein each (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl, in the above definitions of said R³, R⁴, R⁵, R⁶ and R⁸ may be optionally substituted by one to four R⁷ substituents;
wherein R⁷ is (C₁-C₈)alkyl, (C₃-C₁₃)cycloalkyl, (C₆-C₁₀)aryl, 4- to 10-membered heterocyclyl, (C₂-C₆) alkenyl, (C₂-C₆) alkynyl, —O—(C₁-C₈)alkyl, H, OH, F, Cl, Br, I, CN, CF₃, amidino, —C(O)OR⁹, —C(O)R⁹, —SR⁹, —SO₂R⁹, —NO₂, —NR⁹C(O)R¹⁰, —OC(O)R⁹-aryl, —NSO₂R⁹, —SC(O)R⁹, —NC(═S)NR⁹R¹⁰, —O—N═CR⁹, —N═N—R⁹, —C(O)NR⁹R¹⁰, —(CH₂)ₜ—NR⁹R¹⁰, 2- to 10-membered heteroalkyl, 3- to 10-membered heteroalkenyl, 3- to 10-membered heteroalkynyl, —(CH₂)ₜ(C₆-C₁₀ aryl), —(CH₂)ₜ(4 to 10 membered heterocyclic), -(2 to 10 membered heteroalkyl)-(C₆-C₁₀ aryl), -(2 to 10 membered heteroalkyl)-(4 to 10 membered heterocyclyl), —(CH₂)ₜO(CH₂)ᵤOR⁹, and —(CH₂)ₜOR⁹, wherein t is an integer from 0 to 6 and u is an integer from 2 to 6, H or (C₁-C₈)alkyl;
wherein R⁸ is selected from the group consisting of H, OH, CF₃, (C₁-C₈)alkyl, (C₂-C₆)alkenyl, (C₂-C₆)alkynyl, —O—(C₁-C₈)alkyl, (C₃-C₁₀)cycloalkyl, —O—(C₃-C₁₀)cycloalkyl, 4- to 10-membered heterocyclyl, and 4- to 10-membered —O-heterocyclyl;
wherein each R⁹ and R¹⁰ are independently selected from the group consisting of H, (C₁-C₈)alkyl, (C₁-C₈)alkoxyl, —CH₂—(C═O)—O—(C₁-C₈)alkyl, (C₃-C₁₀)cycloalkyl, (C₆-C₁₀)aryl, and 4- to 10-membered heterocyclyl; or R⁹ and R¹⁰ when together attached to the same N, may optionally be taken together with the same nitrogen to form a 5- to 10-membered heterocyclyl ring; with the proviso that where R⁹ and R¹⁰ are both attached to the same nitrogen, then R⁹ and R¹⁰ are not both bonded to the nitrogen directly through an oxygen;
wherein any of the ring members of each (C₃-C₁₃)cycloalkyl or 4- to 10-membered heterocyclyl in R³, R⁴, R⁶, R⁷, R⁸, R⁹ and R¹⁰ may be optionally substituted with an oxo (═O) and wherein any of the (C₁-C₈)alkyl, (C₂-C₆)alkenyl, (C₂-C₆)alkynyl, —O—(C₁-C₈)alkyl, (C₃-C₁₃)cycloalkyl, (C₆-C₁₀)aryl, and 4- to 10-membered heterocyclyl in R⁷, R⁹ and R¹⁰ may be independently further substituted with at least one OH, F, CL, Br, I, CN, CF₃, NO₂, —(C₁-C₈)alkyl, —(C₁-C₈) alkoxyl, COH, or C(O)—(C₁-C₈alkyl).

5. A compound or salt according to claim 3, wherein R¹ is —SOₙ-T-R³, T is as defined above and R³ is a 4- to 10-membered heterocyclic, optionally substituted by one to four substituents selected from R⁷.

6. A compound or salt according to claim 3, wherein T is a bond, R³ is a 4- to 10-membered heterocyclic and R⁷ is an —(C₁-C₈)alkyl.

7. A compound or salt according to claim 4, wherein R³ is a —(CH₂)_{d}(C₃-C₁₃)cycloalkyl, —O—(C₁-C₈)alkyl, —(CH₂)_{d}—(C₆-C₁₀)aryl, —(CH₂)_{d}-(4- to 10-membered heterocyclyl), wherein each R³ (C₃-C₁₀)cycloalkyl, (C₆-C₁₀)aryl, or 4- to 10-membered heterocyclic may be optionally substituted by one to four R⁷substituents.

8. A compound or salt according to claim 3, wherein T is a bond, R³ is a 5-membered heterocyclyl; and R⁷ is (C₁-C₈)alkyl, (C₃-C₁₃)cycloalkyl, (C₆-C₁₀)aryl, or 4- to 10-membered heterocyclyl, —O—(C₁-C₈)alkyl, (C₂-C₆)alkenyl, or (C₂-C₆)alkynyl; wherein each (C₁-C₈)alkyl, (C₃-C₁₃)cycloalkyl, (C₆-C₁₀)aryl, or 4- to 10-membered heterocyclyl, —O—(C₁-C₈)alkyl, (C₂-C₆)alkenyl, or (C₂-C₆)alkynyl may be independently optionally substituted with at least one OH, F, CL, Br, I, CN, CF₃, NO₂, —(C₁-C₈)alkyl, —(C₁-C₈) alkoxyl, COH, or C(O)—(C₁-C₈alkyl).

9. A compound or salt according to claim 4, wherein R³ is a 5-membered heteroaryl; and R⁷ is (C₁-C₈)alkyl, (C₃-C₁₀)cycloalkyl, (C₆-C₁₀)aryl, or 4- to 10-membered heterocyclyl, —O—(C₁-C₈)alkyl, (C₂-C₆)alkenyl, or (C₂-C₆)alkynyl; wherein each (C₁-C₈)alkyl, (C₃-C₁₀)cycloalkyl, (C₆-C₁₀)aryl, or 4- to 10-membered heterocyclyl, (C₁-C₈)alkyl—O—, (C₂-C₆)alkenyl, or (C₂-C₆)alkynyl may be optionally substituted with at least one OH, F, CL, Br, I, CN, CF₃, NO₂, —(C₁-C₈)alkyl, —(C₁-C₈) alkoxyl, COH, or C(O)—(C₁-C₈alkyl);

10. A compound or salt according to claim 1, wherein R² is a 4- to 10-membered heterocyclyl having one or more substituents selected from the group consisting of F, Cl, Br, I.

11. A compound or salt according to claim 3, wherein the group: 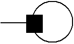
is a nitrogen-containing 4-6 membered heterocyclyl ring optionally substituted with (C₁-C₈)alkyl, (C₃-C₁₀)cycloalkyl, (C₆-C₁₀)aryl, or 4- to 10-membered heterocyclyl; and R² is a (C₆-C₁₀)aryl, or a 4- to 10-membered heterocyclyl having one or more substituents selected from the group consisting of a F, Cl, Br, I.

12. A compound or salt according to claim 4, wherein the group: 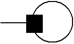
is a nitrogen-containing 4-6 membered heterocyclyl ring optionally substituted by (C₁-C₈)alkyl, (C₃-C₁₀)cycloalkyl, (C₆-C₁₀)aryl, or 4- to 10-membered heterocyclyl; and R² is a (C₆-C₁₀)aryl or 4- to 10-membered heterocyclyl having one or more substituents selected from the group consisting of F, Cl, Br, I.

13. A pharmaceutical composition comprising an amount of active agent effective to modulate cellular proliferation and a pharmaceutically acceptable carrier, said active agent being selected from the group consisting of a compound as defined in claim 1, or a pharmaceutically acceptable salt thereof.

14. A pharmaceutical composition comprising an amount of active agent effective to inhibit protein kinases and a pharmaceutically acceptable carrier, said active agent being selected from the group consisting of a compound as defined in claim 1, or a pharmaceutically acceptable salt thereof.

15. A compound selected from the group consisting of: 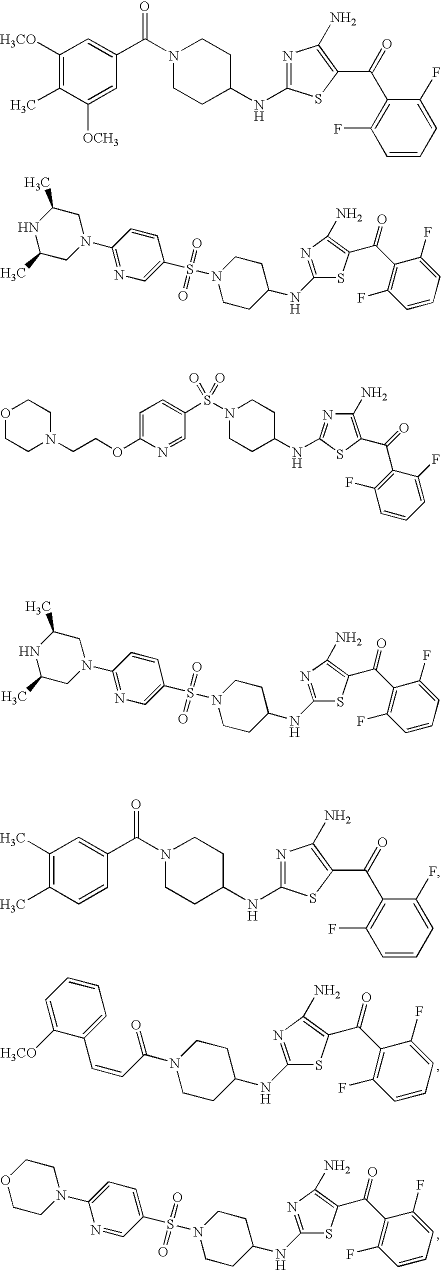 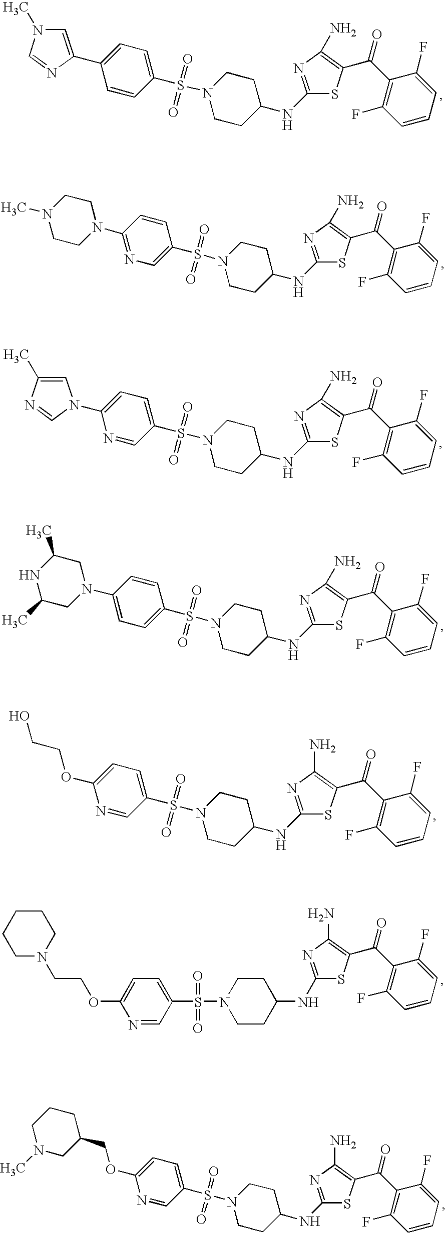 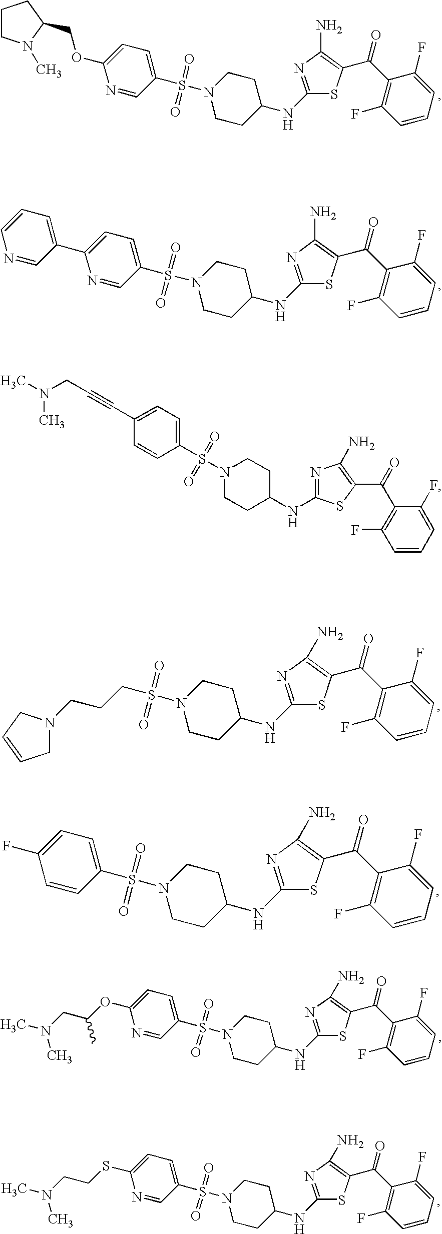 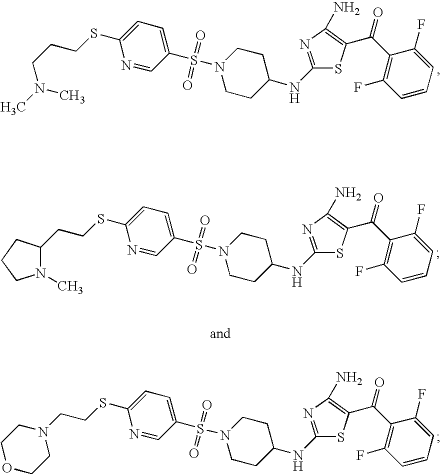
or a pharmaceutically acceptable salt of such compound.
